SEQUENCE LISTING (101 sequences)

SEQ ID NO: 1 - DNA, 18 bp, Artificial Sequence
  source: /note=“Description of Artificial Sequence Synthetic primer”
gcgacaggtgactgcagc

SEQ ID NO: 2 - DNA, 36 bp, Artificial Sequence
  source: /note=“Description of Artificial Sequence Synthetic oligonucleotide”
  modified_base (18)..(18): 2′-deoxyinosine
cacatcacgtcgtagtcngctgcagtcacctgtcgc

SEQ ID NO: 3 - DNA, 36 bp, Artificial Sequence; identical to SEQ ID NO: 2
  source: /note=“Description of Artificial Sequence Synthetic oligonucleotide”
  modified_base (18)..(18): 2′-deoxyinosine

SEQ ID NO: 4 - DNA, 19 bp, Artificial Sequence
  source: /note=“Description of Artificial Sequence Synthetic primer”
gcgacaggtgactgcagct

SEQ ID NO: 5 - DNA, 36 bp, Artificial Sequence
  source: /note=“Description of Artificial Sequence Synthetic oligonucleotide”
gcgacaggtgactgcagctgactacgacgtgatgtg

SEQ ID NO: 6 - DNA, 36 bp, Artificial Sequence
  source: /note=“Description of Artificial Sequence Synthetic oligonucleotide”
  modified_base (17)..(17): 2′-deoxyinosine
cacatcacgtcgtagtnagctgcagtcacctgtcgc

SEQ ID NO: 7 - DNA, 18 bp, Artificial Sequence
  source: /note=“Description of Artificial Sequence Synthetic oligonucleotide”
gctgcagtcacctgtcgc

SEQ ID NO: 8 - DNA, 19 bp, Artificial Sequence
  source: /note=“Description of Artificial Sequence Synthetic primer”
gcgacaggtgactgcagct

SEQ ID NO: 9 - DNA, 18 bp, Artificial Sequence
  source: /note=“Description of Artificial Sequence Synthetic oligonucleotide”
  modified_base (17)..(17): 2′-deoxyinosine
cacatcacgtcgtagtna

SEQ ID NO: 10 - DNA, 18 bp, Artificial Sequence
  source: /note=“Description of Artificial Sequence Synthetic primer”
gcgacaggtgactgcagc

SEQ ID NO: 11 - DNA, 36 bp, Artificial Sequence; identical to SEQ ID NO: 2
  source: /note=“Description of Artificial Sequence Synthetic oligonucleotide”
  modified_base (18)..(18): 2′-deoxyinosine

SEQ ID NO: 12 - DNA, 18 bp, Artificial Sequence
  source: /note=“Description of Artificial Sequence Synthetic oligonucleotide”
cgactacgacgtgatgtg

SEQ ID NO: 13 - DNA, 36 bp, Artificial Sequence; identical to SEQ ID NO: 2
  source: /note=“Description of Artificial Sequence Synthetic oligonucleotide”
  modified_base (18)..(18): Deoxyinosine

SEQ ID NO: 14 - DNA, 19 bp, Artificial Sequence
  source: /note=“Description of Artificial Sequence Synthetic primer”
gcgacaggtgactgcagct

SEQ ID NO: 15 - DNA, 36 bp, Artificial Sequence; identical to SEQ ID NO: 5
  source: /note=“Description of Artificial Sequence Synthetic oligonucleotide”

SEQ ID NO: 16 - DNA, 18 bp, Artificial Sequence
  source: /note=“Description of Artificial Sequence Synthetic oligonucleotide”
tgactacgacgtgatgtg

SEQ ID NO: 17 - DNA, 36 bp, Artificial Sequence; identical to SEQ ID NO: 6
  source: /note=“Description of Artificial Sequence Synthetic oligonucleotide”
  modified_base (17)..(17): 2′-deoxyinosine

SEQ ID NO: 18 - DNA, 18 bp, Artificial Sequence
  source: /note=“Description of Artificial Sequence Synthetic oligonucleotide”
gctgcagtcacctgtcgc

SEQ ID NO: 19 - DNA, 19 bp, Artificial Sequence
  source: /note=“Description of Artificial Sequence Synthetic primer”
gcgacaggtgactgcagct

SEQ ID NO: 20 - DNA, 18 bp, Artificial Sequence
  source: /note=“Description of Artificial Sequence Synthetic oligonucleotide”
  modified_base (17)..(17): 2′-deoxyinosine
cacatcacgtcgtagtna

SEQ ID NO: 21 - DNA, 17 bp, Artificial Sequence
  source: /note=“Description of Artificial Sequence Synthetic oligonucleotide”
cactacgacgtgatgtg

SEQ ID NO: 22 - DNA, 18 bp, Artificial Sequence
  source: /note=“Description of Artificial Sequence Synthetic primer”
gcgacaggtgactgcagc

SEQ ID NO: 23 - DNA, 37 bp, Artificial Sequence
  source: /note=“Description of Artificial Sequence Synthetic oligonucleotide”
  modified_base (18)..(18): 2′-deoxyinosine
cacatcacgtcgtagtcnagctgcagtcacctgtcgc

SEQ ID NO: 24 - DNA, 37 bp, Artificial Sequence
  source: /note=“Description of Artificial Sequence Synthetic oligonucleotide”
  modified_base (18)..(18): 2′-deoxyinosine
cacatcacgtcgtagtcnggctgcagtcacctgtcgc

SEQ ID NO: 25 - DNA, 37 bp, Artificial Sequence; identical to SEQ ID NO: 23
  source: /note=“Description of Artificial Sequence Synthetic oligonucleotide”
  modified_base (18)..(18): 2′-deoxyinosine

SEQ ID NO: 26 - DNA, 37 bp, Artificial Sequence; identical to SEQ ID NO: 23
  source: /note=“Description of Artificial Sequence Synthetic oligonucleotide”
  modified_base (18)..(18): 2′-deoxyinosine

SEQ ID NO: 27 - DNA, 19 bp, Artificial Sequence
  source: /note=“Description of Artificial Sequence Synthetic oligonucleotide”
tcgactacgacgtgatgtg

SEQ ID NO: 28 - DNA, 19 bp, Artificial Sequence
  source: /note=“Description of Artificial Sequence Synthetic oligonucleotide”
ccgactacgacgtgatgtg

SEQ ID NO: 29 - DNA, 19 bp, Artificial Sequence
  source: /note=“Description of Artificial Sequence Synthetic oligonucleotide”
acgactacgacgtgatgtg

SEQ ID NO: 30 - DNA, 19 bp, Artificial Sequence
  source: /note=“Description of Artificial Sequence Synthetic oligonucleotide”
gcgactacgacgtgatgtg

SEQ ID NO: 31 - DNA, 37 bp, Artificial Sequence; identical to SEQ ID NO: 23
  source: /note=“Description of Artificial Sequence Synthetic oligonucleotide”
  modified_base (18)..(18): Deoxyinosine

SEQ ID NO: 32 - DNA, 19 bp, Artificial Sequence
  source: /note=“Description of Artificial Sequence Synthetic primer”
gcgacaggtgactgcagct

SEQ ID NO: 33 - DNA, 36 bp, Artificial Sequence; identical to SEQ ID NO: 5
  source: /note=“Description of Artificial Sequence Synthetic oligonucleotide”

SEQ ID NO: 34 - DNA, 19 bp, Artificial Sequence
  source: /note=“Description of Artificial Sequence Synthetic oligonucleotide”
tcgactacgacgtgatgtg

SEQ ID NO: 35 - DNA, 18 bp, Artificial Sequence
  source: /note=“Description of Artificial Sequence Synthetic oligonucleotide”
gctgcagtcacctgtcgc

SEQ ID NO: 36 - DNA, 19 bp, Artificial Sequence
  source: /note=“Description of Artificial Sequence Synthetic primer”
gcgacaggtgactgcagct

SEQ ID NO: 37 - DNA, 19 bp, Artificial Sequence
  source: /note=“Description of Artificial Sequence Synthetic oligonucleotide”
  modified_base (17)..(17): 2′-deoxyinosine
cacatcacgtcgtagtnga

SEQ ID NO: 38 - DNA, 18 bp, Artificial Sequence
  source: /note=“Description of Artificial Sequence Synthetic oligonucleotide”
ccactacgacgtgatgtg

SEQ ID NO: 39 - DNA, 18 bp, Artificial Sequence
  source: /note=“Description of Artificial Sequence Synthetic primer”
gcgacaggtgactgcagc

SEQ ID NO: 40 - DNA, 36 bp, Artificial Sequence
  source: /note=“Description of Artificial Sequence Synthetic oligonucleotide”
cacatcacgtcgtagtcagctgcagtcacctgtcgc

SEQ ID NO: 41 - DNA, 36 bp, Artificial Sequence
  source: /note=“Description of Artificial Sequence Synthetic oligonucleotide”
cacatcacgtcgtagtcggctgcagtcacctgtcgc

SEQ ID NO: 42 - DNA, 36 bp, Artificial Sequence
  source: /note=“Description of Artificial Sequence Synthetic oligonucleotide”
cacatcacgtcgtagtctgctgcagtcacctgtcgc

SEQ ID NO: 43 - DNA, 36 bp, Artificial Sequence
  source: /note=“Description of Artificial Sequence Synthetic oligonucleotide”
cacatcacgtcgtagtccgctgcagtcacctgtcgc

SEQ ID NO: 44 - DNA, 78 bp, Artificial Sequence
  source: /note=“Description of Artificial Sequence Synthetic oligonucleotide”
  modified_base (36)..(36): 2′-deoxyinosine
  modified_base (58)..(58): Tamra-dT
tcgactacgacgtgacttttagtcacgtcgtagtcnagctgcagtcacctgctgcttnttgcagcaggtgactgcagc

SEQ ID NO: 45 - DNA, 79 bp, Artificial Sequence
  source: /note=“Description of Artificial Sequence Synthetic oligonucleotide”
  modified_base (36)..(36): 2′-deoxyinosine
  modified_base (58)..(58): Tamra-dT
tcgactacgacgtgacttttagtcacgtcgtagtcnagctgcagtcacctgctgcttnttgcagcaggtgactgcagct

SEQ ID NO: 46 - DNA, 42 bp, Artificial Sequence
  source: /note=“Description of Artificial Sequence Synthetic oligonucleotide”
  modified_base (21)..(21): Tamra-dT phosphate
gctgcagtcacctgctgcttnttgcagcaggtgactgcagct

SEQ ID NO: 47 - DNA, 38 bp, Artificial Sequence
  source: /note=“Description of Artificial Sequence Synthetic oligonucleotide”
  modified_base (36)..(36): 2′-deoxyinosine
ccgactacgacgtgacttttagtcacgtcgtagtcnga

SEQ ID NO: 48 - DNA, 78 bp, Artificial Sequence; identical to SEQ ID NO: 44
  source: /note=“Description of Artificial Sequence Synthetic oligonucleotide”
  modified_base (36)..(36): 2′-deoxyinosine
  modified_base (58)..(58): Tamra-dT

SEQ ID NO: 49 - DNA, 38 bp, Artificial Sequence; identical to SEQ ID NO: 47
  source: /note=“Description of Artificial Sequence Synthetic oligonucleotide”
  modified_base (36)..(36): 2′-deoxyinosine

SEQ ID NO: 50 - DNA, 58 bp, Artificial Sequence
  source: /note=“Description of Artificial Sequence Synthetic oligonucleotide”
  modified_base (16)..(16): 2′-deoxyinosine
  modified_base (38)..(38): Tamra-dT
agtcacgtcgtagtcnagctgcagtcacctgctgcttnttgcagcaggtgactgcagc

SEQ ID NO: 51 - DNA, 17 bp, Artificial Sequence
  source: /note=“Description of Artificial Sequence Synthetic oligonucleotide”
tcgactacgacgtgact

SEQ ID NO: 52 - DNA, 18 bp, Artificial Sequence
  source: /note=“Description of Artificial Sequence Synthetic oligonucleotide”
  modified_base (16)..(16): 2′-deoxyinosine
agtcacgtcgtagtcnga

SEQ ID NO: 53 - DNA, 17 bp, Artificial Sequence
  source: /note=“Description of Artificial Sequence Synthetic oligonucleotide”
ccgactacgacgtgact

SEQ ID NO: 54 - DNA, 78 bp, Artificial Sequence
  source: /note=“Description of Artificial Sequence Synthetic oligonucleotide”
  modified_base (35)..(35): 2′-deoxyinosine
  modified_base (57)..(57): Tamra-dT
cgactacgacgtgacttttagtcacgtcgtagtcnagctgcagtcacctgctgcttnttgcagcaggtgactgcagct

SEQ ID NO: 55 - DNA, 37 bp, Artificial Sequence
  source: /note=“Description of Artificial Sequence Synthetic oligonucleotide”
  modified_base (35)..(35): 2′-deoxyinosine
cgactacgacgtgacttttagtcacgtcgtagtcnaa

SEQ ID NO: 56 - DNA, 78 bp, Artificial Sequence
  source: /note=“Description of Artificial Sequence Synthetic oligonucleotide”
  modified_base (35)..(35): 2′-deoxyinosine
  modified_base (52)..(52): Tamra-dT
  modified_base (57)..(57): Biotin-dT
cgactacgacgtgacttttagtcacgtcgtagtcnagctgcagtcacctgcngcttnttgcagcaggtgactgcagct

SEQ ID NO: 57 - DNA, 38 bp, Artificial Sequence; identical to SEQ ID NO: 47
  source: /note=“Description of Artificial Sequence Synthetic oligonucleotide”
  modified_base (36)..(36): 2′-deoxyinosine

SEQ ID NO: 58 - DNA, 62 bp, Artificial Sequence
  source: /note=“Description of Artificial Sequence Synthetic oligonucleotide”
  modified_base (60)..(60): 2′-deoxyinosine
acgagtgacctggttttttttttttttttttttttttttttttttttaccaggtcactcntg

SEQ ID NO: 59 - DNA, 58 bp, Artificial Sequence
  source: /note=“Description of Artificial Sequence Synthetic oligonucleotide”
  modified_base (16)..(16): 2′-deoxyinosine
  modified_base (33)..(33): Tamra-dT
  modified_base (38)..(38): Biotin-dT
agtcacgtcgtagtcnagctgcagtcacctgcngcttnttgcagcaggtgactgcagc

SEQ ID NO: 60 - DNA, 18 bp, Artificial Sequence
  source: /note=“Description of Artificial Sequence Synthetic oligonucleotide”
  modified_base (16)..(16): 2′-deoxyinosine
agtcacgtcgtagtcnaa

SEQ ID NO: 61 - DNA, 67 bp, Artificial Sequence
  source: /note=“Description of Artificial Sequence Synthetic oligonucleotide”
  modified_base (16)..(16): 2′-deoxyinosine
agtcacgtcgtagtcnagctgcagtcacctgctgcttttttttttttttgcagcaggtgactgcagc

SEQ ID NO: 62 - DNA, 67 bp, Artificial Sequence; identical to SEQ ID NO: 61
  source: /note=“Description of Artificial Sequence Synthetic oligonucleotide”
  modified_base (16)..(16): 2′-deoxyinosine

SEQ ID NO: 63 - DNA, 58 bp, Artificial Sequence
  source: /note=“Description of Artificial Sequence Synthetic oligonucleotide”
  modified_base (16)..(16): 2′-deoxyinosine
  modified_base (38)..(38): 5′′-thiophosphate-Sp9-Sp9-Sp9-5-methylC
agtcacgtcgtagtcnagctgcagtcacctgctgcttcttgcagcaggtgactgcagc

SEQ ID NO: 64 - DNA, 58 bp, Artificial Sequence; identical to SEQ ID NO: 63
  source: /note=“Description of Artificial Sequence Synthetic oligonucleotide”
  modified_base (16)..(16): 2′-deoxyinosine
  modified_base (38)..(38): 5′′-phosphate-Sp9-Sp9-Sp9-5-methylC

SEQ ID NO: 65 - DNA, 59 bp, Artificial Sequence
  source: /note=“Description of Artificial Sequence Synthetic oligonucleotide”
  modified_base (16)..(16): 2′-deoxyinosine
  modified_base (38)..(38): 5′′-thiophosphate-Sp9-Sp9-Sp9-5-methylC
agtcacgtcgtagtcnagctgcagtcacctgctgcttcttgcagcaggtgactgcagct

SEQ ID NO: 66 - DNA, 59 bp, Artificial Sequence
  source: /note=“Description of Artificial Sequence Synthetic oligonucleotide”
  modified_base (38)..(38): 5′′-thiophosphate-Sp9-Sp9-Sp9-5-methylC
agtcacgtcgtagtcgagctgcagtcacctgctgcttcttgcagcaggtgactgcagct

SEQ ID NO: 67 - DNA, 18 bp, Artificial Sequence
  source: /note=“Description of Artificial Sequence Synthetic oligonucleotide”
  modified_base (16)..(16): 2′-deoxyinosine
agtcacgtcgtagtcnaa

SEQ ID NO: 68 - DNA, 18 bp, Artificial Sequence
  primer sequence - figure 51
gcgacaggtgactgcagc

SEQ ID NO: 69 - DNA, 18 bp, Artificial Sequence
  template sequence - figure 51
gctgcagtcacctgtcgc

SEQ ID NO: 70 - DNA, 43 bp, Artificial Sequence
  source: /note=“Description of Artificial Sequence Hairpin blunt end - figure 58”
  modified_base (17)..(17): TAMRA-dT
agctgcagtcacctgcngctttttgcagcaggtgactgcagct

SEQ ID NO: 71 - DNA, 18 bp, Artificial Sequence
  source: /note=“Description of Artificial Sequence Inosine-strand - figure 58”
  modified_base (16)..(16): 2′-deoxyinosine
agtcacgtcgtagtcnca

SEQ ID NO: 72 - DNA, 18 bp, Artificial Sequence
  Helper strand - figure 58
tgcgactacgacgtgact

SEQ ID NO: 73 - DNA, 44 bp, Artificial Sequence
  source: /note=“Description of Artificial Sequence Hairpin 1 base overhang T- figure 62”
  modified_base (20)..(20): TAMRA-dT
tgctgtagtcaccatctgcntttttagcagatggtgactacagc

SEQ ID NO: 74 - DNA, 44 bp, Artificial Sequence
  source: /note=“Description of Artificial Sequence Hairpin 1 base overhang C - figure 62”
  modified_base (20)..(20): TAMRA-dT
cgctgtagtcaccatctgcntttttagcagatggtgactacagc

SEQ ID NO: 75 - DNA, 45 bp, Artificial Sequence
  source: /note=“Description of Artificial Sequence Hairpin 2 base overhang - figure 62”
  modified_base (21)..(21): TAMRA-dT
acgctgtagtcaccatctgcntttttagcagatggtgactacagc

SEQ ID NO: 76 - DNA, 46 bp, Artificial Sequence
  source: /note=“Description of Artificial Sequence Hairpin 3 base overhang - figure 62”
  modified_base (22)..(22): TAMRA-dT
cacgctgtagtcaccatctgcntttttagcagatggtgactacagc

SEQ ID NO: 77 - DNA, 47 bp, Artificial Sequence
  source: /note=“Description of Artificial Sequence Hairpin 4 base overhang - figure 62”
  modified_base (23)..(23): TAMRA-dT
tcacgctgtagtcaccatctgcntttttagcagatggtgactacagc

SEQ ID NO: 78 - DNA, 18 bp, Artificial Sequence
  source: /note=“Description of Artificial Sequence Inosine strand - figure 62”
  modified_base (16)..(16): 2′-deoxyinosine
agtcacgtcgtagtcncg

SEQ ID NO: 79 - DNA, 19 bp, Artificial Sequence
  Helper strand - overhang T - figure 62
acgcgactacgacgtgact

SEQ ID NO: 80 - DNA, 19 bp, Artificial Sequence
  Helper strand - overhang C - figure 62
gcgcgactacgacgtgact

SEQ ID NO: 81 - DNA, 20 bp, Artificial Sequence
  Helper strand - 2 base overhang - figure 62
gtcgcgactacgacgtgact

SEQ ID NO: 82 - DNA, 21 bp, Artificial Sequence
  Helper strand - 3 base overhang - figure 62
gtgcgcgactacgacgtgact

SEQ ID NO: 83 - DNA, 22 bp, Artificial Sequence
  Helper strand - 4 base overhang - figure 62
gtgacgcgactacgacgtgact

SEQ ID NO: 84 - DNA, 43 bp, Artificial Sequence
  Hairpin for ligation - figure 64C
  TAMRA-dT (19)..(19)
gctgtagtcaccatctgcntttttagcagatggtgactacagc

SEQ ID NO: 85 - DNA, 18 bp, Artificial Sequence
  U-strand - figure 64C
  2′-deoxyuridine (2)..(2)
gncgactacgacgtgact

SEQ ID NO: 86 - DNA, 17 bp, Artificial Sequence
  Helper strand - figure 64C
  2′,3′-dideoxycytidine (17)..(17)
gtcacgtcgtagtcgac

SEQ ID NO: 87 - DNA, 44 bp, Artificial Sequence
  Hairpin for ligation - figure 65C
  TAMRA-dT (19)..(19)
gctgtagtcaccatctgcntttttagcagatggtgactacagcg

SEQ ID NO: 88 - DNA, 18 bp, Artificial Sequence
  In-strand - figure 65C
  2′-deoxyinosine (16)..(16)
agtcacgtcgtagtcnac

SEQ ID NO: 89 - DNA, 17 bp, Artificial Sequence
  Helper strand - figure 65C
tcgactacgacgtgact

SEQ ID NO: 90 - DNA, 43 bp, Artificial Sequence; identical to SEQ ID NO: 84
  Hairpin for ligation - figure 66C
  TAMRA-dT (19)..(19)

SEQ ID NO: 91 - DNA, 19 bp, Artificial Sequence
  U-strand - figure 66C
  2′-deoxyuridine (18)..(18)
aagtcacgtcgtagtcgnc

SEQ ID NO: 92 - DNA, 18 bp, Artificial Sequence
  Helper strand - figure 66C
gacgactacgacgtgact

SEQ ID NO: 93 - DNA, 44 bp, Artificial Sequence; identical to SEQ ID NO: 74
  Hairpin for ligation - figure 67C
  TAMRA-dT (20)..(20)

SEQ ID NO: 94 - DNA, 18 bp, Artificial Sequence
  U-strand
  2′-deoxyuridine (2)..(2)
gncgactacgacgtgact

SEQ ID NO: 95 - DNA, 17 bp, Artificial Sequence
  Helper strand - figure 67C
  2′,3′-dideoxyadenosine (17)..(17)
agtcacgtcgtagtcga

SEQ ID NO: 96 - DNA, 44 bp, Artificial Sequence; identical to SEQ ID NO: 74
  Hairpin for ligation - figure 68C
  TAMRA-dT (20)..(20)

SEQ ID NO: 97 - DNA, 18 bp, Artificial Sequence
  U-strand - figure 68C
  d′-deoxyuridine (2)..(2)
gncgactacgacgtgact

SEQ ID NO: 98 - DNA, 17 bp, Artificial Sequence
  Helper strand - figure 68C
  2′,3′-dideoxyadenosine (17)..(17)
agtcacgtcgtagtcga

SEQ ID NO: 99 - DNA, 45 bp, Artificial Sequence
  Hairpin for ligation - figure 69C
  TAMRA-dT (20)..(20)
cgctgtagtcaccatctgcntttttagcagatggtgactacagcg

SEQ ID NO: 100 - DNA, 18 bp, Artificial Sequence
  U-strand - figure 69C
  2′-deoxyuridine (2)..(2)
gncgactacgacgtgact

SEQ ID NO: 101 - DNA, 17 bp, Artificial Sequence
  Helper strand - figure 69C
  2′,3′-dideoxycytidine (17)..(17)
gtcacgtcgtagtcgac

(19) United States
(12) Patent Application Publication
(10) Pub. No.: US 2022/0396818 A1
(43) Pub. Date: Dec. 15, 2022

(54) POLYNUCLEOTIDE SYNTHESIS METHOD, KIT AND SYSTEM

(71) Applicant: Oxford Nanopore Technologies PLC, Oxford (GB)

(72) Inventors: John Milton, Oxford (GB); Sobia Nayyar, Oxford (GB); Jan Riedl, Oxford (GB); Ryosuke Ogaki, Oxford (GB); Marc George Wilkinson, Oxford (GB)

(73) Assignee: Oxford Nanopore Technologies PLC, Oxford (GB)

(21) Appl. No.: 17/641,493

(22) PCT Filed: Sep. 10, 2020
(86) PCT No.: PCT/GB2020/052172

§ 371 Date: Mar. 9, 2022

(30) Foreign Application Priority Data
Sep. 10, 2019 (GB) 1913039.2

(51) Int. Cl. C12P 19/34; C12Q 1/6813; C12Q 1/6844

(52) U.S. Cl. CPC C12P 19/34; C12Q 1/6813; C12Q 1/6844

(57) ABSTRACT

The invention relates to new methods for synthesising polynucleotide molecules according to a predefined nucleotide sequence. The invention also relates to methods for the assembly of synthetic polynucleotides following synthesis, as well as systems and kits for performing the synthesis and/or assembly methods.

### RELATED APPLICATIONS

This application is a national stage filing under 35 U.S.C. § 371 of international application number PCT/GB2020/052172, filed Sep. 10, 2020, which claims the benefit of United Kingdom application number GB 1913039.2, filed Sep. 10, 2019, the entire content of each of which is herein incorporated by reference.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Feb. 14, 2022, is named O036670122US00-SEQ-KZM and is 30424 bytes in size.

### FIELD OF THE INVENTION

The invention relates to new methods for synthesising polynucleotide molecules according to a predefined nucleotide sequence. The invention also relates to methods for the assembly of synthetic polynucleotides following synthesis, as well as systems and kits for performing the synthesis and/or assembly methods.

### BACKGROUND TO THE INVENTION

Two primary methods exist for the synthesis and assembly of polynucleotide molecules, particularly DNA.

Phosphoramidite chemistry is a synthetic approach that assembles monomers of chemically activated T, C, A or G into oligonucleotides of approximately 100/150 bases in length via a stepwise process. The chemical reaction steps are highly sensitive and the conditions alternate between fully anhydrous (complete absence of water), aqueous oxidative and acidic conditions (Roy and Caruthers, Molecules, 2013, 18, 14268-14284). If the reagents from the previous reaction step have not been completely removed this will be detrimental to future steps of synthesis. Accordingly, this synthesis method is limited to the production of polynucleotides of length of approximately 100 nucleotides.

The Polymerase Synthetic approach uses a polymerase to synthesise a complementary strand to a DNA template using T, C, A and G triphosphates. The reaction conditions are aqueous and mild and this approach can be used to synthesise DNA polynucleotides which are many thousands of bases in length. The main disadvantage of this method is that single- and double-stranded DNA cannot be synthesised de novo by this method, it requires a DNA template from which a copy is made. (Kosuri and Church, Nature Methods, 2014, 11, 499-507).

Thus previous methods cannot be used to synthesise double-stranded DNA de novo without the aid of a pre-existing template molecule which is copied.

The inventors have developed new methodologies by which single- and double-stranded polynucleotide molecules can be synthesised de novo in a stepwise manner without the need to copy a pre-existing template molecule. Such methods also avoid the extreme conditions associated with phosphoramidite chemistry techniques and in contrast are carried out under mild, aqueous conditions around neutral pH. Such methods also enable de novo synthesis of single- or double-stranded polynucleotide molecules with a potential 10⁸ improvement on current synthesis methods with nucleotide lengths of >100 mers to full genomes, providing a wide range of possibly applications in synthetic biology.

### SUMMARY OF THE INVENTION

The invention provides an in vitro method of synthesising a double-stranded polynucleotide wherein at least one strand has a predefined sequence, the method comprising performing cycles of synthesis wherein in each cycle one strand of a double-stranded polynucleotide is extended by the incorporation of one or more nucleotides in a first ligation reaction by the action of an enzyme having ligase activity, and the opposite strand of the double-stranded polynucleotide is extended by the incorporation of one or more nucleotides in a second ligation reaction by the action of an enzyme having ligase activity, wherein both strands are extended at the same terminal end of the double-stranded polynucleotide.

In any of the methods of the invention described herein the methods provide for the synthesis of a double-stranded or single-stranded oligonucleotide. Thus all references herein to the synthesis of a double-stranded or single-stranded polynucleotide using any of the methods of the invention apply mutatis mutandis to the synthesis of a double-stranded or single-stranded oligonucleotide.

In the method of the invention: (i) at least one strand may have a predefined sequence, and wherein the nucleotides that are incorporated into said strand are nucleotides of the predefined sequence; or (ii) both strands may have a predefined sequence, and wherein the nucleotides that are incorporated into one strand are nucleotides of the predefined sequence of that strand, and wherein the nucleotides that are incorporated into the opposite strand are nucleotides of the predefined sequence of the opposite strand.

In such a method, in a cycle of synthesis:
a) the 3′ end of one strand may be extended by the incorporation of one or more nucleotides, and then
b) the 5′ end of the opposite strand may be extended by the incorporation of one or more nucleotides. These methods may be performed in accordance with the exemplary synthesis methods of the invention versions 1, 2, 3 and 4 as described herein.

Alternatively, in such a method, in a cycle of synthesis:
a) the 5′ end of one strand may be extended by the incorporation of one or more nucleotides, and then
b) the 3′ end of the opposite strand may be extended by the incorporation of one or more nucleotides. These methods may be performed in accordance with the exemplary synthesis methods of the invention versions 5 and 6 as described herein.

In any such method in a cycle of synthesis one strand may be extended by the incorporation of a first nucleotide, and the opposite strand may be extended by the incorporation of a second nucleotide which pairs with the first nucleotide. These methods may be performed in accordance with the exemplary synthesis methods of the invention versions 3, 4, 5 and 6 as described herein.

Alternatively, in any such method in a cycle of synthesis one strand may be extended by the incorporation of two nucleotides, and the opposite strand may be extended by the incorporation of two nucleotides, thereby forming two nucleotide pairs. These methods may be performed in accordance with the exemplary synthesis methods of the invention versions 1 and 2 as described herein.

In any of the above-described methods each cycle of synthesis may comprise steps comprising:

(1) providing a double-stranded scaffold polynucleotide;

(2) extending a first strand of the scaffold polynucleotide by incorporating one or more nucleotides into the first strand;

(3) subjecting the first strand to a cleavage step, wherein the one or more nucleotides are retained in the first strand of the scaffold polynucleotide following cleavage;

(4) extending the second strand of the scaffold polynucleotide by incorporating one or more nucleotides into the second strand; and

(5) subjecting the second strand to a cleavage step, wherein the one or more nucleotides are retained in the second strand of the scaffold polynucleotide following cleavage. The cleavage site in steps (3) and (5) may be defined by a polynucleotide sequence in the cleaved strand comprising a universal nucleotide. In such a method the double-stranded scaffold polynucleotide is provided with a ligation end and an opposite end; and wherein in steps (2) and (4) the one or more nucleotides of the predefined sequence are provided by first and second polynucleotide ligation molecules which are ligated to the ligation end of the scaffold polynucleotide by the action of the enzyme, and wherein a polynucleotide ligation molecule comprises a universal nucleotide, wherein upon ligation of a polynucleotide ligation molecule to the scaffold polynucleotide a strand of the scaffold polynucleotide is extended and a cleavage site defined by the universal nucleotide is created.

In the above-described methods a polynucleotide ligation molecule may be a double-stranded polynucleotide molecule comprising a synthesis strand and a helper strand hybridised thereto, and further comprising a complementary ligation end, the ligation end comprising:
(i) in the synthesis strand: (a) the one or more nucleotides for extending the scaffold polynucleotide positioned at the terminal end of the synthesis strand, and (b) the universal nucleotide; and
(ii) in the helper strand a non-ligatable terminal nucleotide.

A method involving a polynucleotide ligation molecule may be performed as follows:
(A) in step (1) the double-stranded scaffold polynucleotide is provided with a single base overhang, with the terminal nucleotide of the second strand overhanging the terminal nucleotide of the first strand;
(B) in step (2), in the first polynucleotide ligation molecule the terminal nucleotide of the synthesis strand occupies position n, wherein position n is the nucleotide position which is occupied by the first nucleotide to be added to the terminal end of the first strand of the scaffold polynucleotide in step (2); the penultimate nucleotide of the synthesis strand occupies position n+1, wherein position n+1 is the nucleotide position which is occupied by the second nucleotide to be added to the terminal end of the first strand of the scaffold polynucleotide in step (2); the universal nucleotide occupies position n+2 in the synthesis strand and is paired with the penultimate nucleotide of the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand;
(C) in step (3) the first strand of the ligated scaffold polynucleotide is cleaved between positions n+1 and n+2 whereupon the universal nucleotide is removed from the scaffold polynucleotide and the first and second nucleotides of the first polynucleotide ligation molecule are retained in the scaffold polynucleotide, and whereupon a single base overhang is created in the scaffold polynucleotide with the terminal nucleotide of the first strand overhanging the terminal nucleotide of the second strand;
(D) in step (4), in the second polynucleotide ligation molecule the terminal nucleotide of the synthesis strand occupies position n+1, wherein position n+1 is the nucleotide position which is occupied by the first nucleotide to be added to the terminal end of the second strand of the scaffold polynucleotide in step (4) and will be paired with the second nucleotide which was added to the terminal end of the first strand in step (2); the penultimate nucleotide of the synthesis strand occupies position n+2, wherein position n+2 is the nucleotide position which is occupied by the second nucleotide to be added to the terminal end of the second strand of the scaffold polynucleotide in step (4); the universal nucleotide occupies position n+3 in the synthesis strand and is paired with the penultimate nucleotide of the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand; and
(E) in step (5) the second strand of the ligated scaffold polynucleotide is cleaved between positions n+2 and n+3 whereupon the universal nucleotide is removed from the scaffold polynucleotide and the first and second nucleotides of the second polynucleotide ligation molecule are retained in the scaffold polynucleotide, and whereupon a single base overhang is created in the scaffold polynucleotide with the terminal nucleotide of the second strand overhanging the terminal nucleotide of the first strand. Such a method may be performed in accordance with the exemplary synthesis method of the invention version 1 as described herein.

A method involving a polynucleotide ligation molecule may alternatively be performed as follows:
(A) in step (1) the double-stranded scaffold polynucleotide is provided with a single base overhang, with the terminal nucleotide of the second strand overhanging the terminal nucleotide of the first strand;
(B) in step (2), in the first polynucleotide ligation molecule the terminal nucleotide of the synthesis strand occupies position n, wherein position n is the nucleotide position which is occupied by the first nucleotide to be added to the terminal end of the first strand of the scaffold polynucleotide in step (2); the penultimate nucleotide of the synthesis strand occupies position n+1, wherein position n+1 is the nucleotide position which is occupied by the second nucleotide to be added to the terminal end of the first strand of the scaffold polynucleotide in step (2); the universal nucleotide occupies position n+2 in the synthesis strand and is paired with the penultimate nucleotide of the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand;
(C) in step (3) the first strand of the ligated scaffold polynucleotide is cleaved between positions n+1 and n+2 whereupon the universal nucleotide is removed from the scaffold polynucleotide and the first and second nucleotides of the first polynucleotide ligation molecule are retained in the scaffold polynucleotide, and whereupon a single base overhang is created in the scaffold polynucleotide with the terminal nucleotide of the first strand overhanging the terminal nucleotide of the second strand;
(D) in step (4), in the second polynucleotide ligation molecule the terminal nucleotide of the synthesis strand occupies position n+1, wherein position n+1 is the nucleotide position which is occupied by the first nucleotide to be added to the terminal end of the second strand of the scaffold polynucleotide in step (4) and will be paired with the second nucleotide which was added to the terminal end of the first strand in step (2); the penultimate nucleotide of the synthesis strand occupies position n+2, wherein position n+2 is the nucleotide position which is occupied by the second nucleotide to be added to the terminal end of the second strand of the scaffold polynucleotide in step (4); the universal nucleotide occupies position n+4 in the synthesis strand and is paired with the nucleotide in the helper strand which is next to the penultimate nucleotide of the helper strand in the direction distal to the complementary ligation end; the terminal nucleotide of the helper strand is a non-ligatable nucleotide; and the complementary ligation end is provided with a single base overhang, with the terminal nucleotide of the synthesis strand overhanging the terminal nucleotide of the helper strand; and
(E) in step (5) the second strand of the ligated scaffold polynucleotide is cleaved between positions n+2 and n+3 whereupon the universal nucleotide is removed from the scaffold polynucleotide and the first and second nucleotides of the second polynucleotide ligation molecule are retained in the scaffold polynucleotide, and whereupon a single base overhang is created in the scaffold polynucleotide with the terminal nucleotide of the second strand overhanging the terminal nucleotide of the first strand. Such a method may be performed in accordance with exemplary synthesis method of the invention version 2 as described herein.

In any of the immediately above-described methods which may be performed in accordance with exemplary synthesis method of the invention version 2 as described herein, the methods may comprise variant methods wherein:
(i) in step (4), in the second polynucleotide ligation molecule the terminal nucleotide of the synthesis strand occupies position n+1, wherein position n+1 is the nucleotide position which is occupied by the first nucleotide to be added to the terminal end of the second strand of the scaffold polynucleotide in step (4) and will be paired with the second nucleotide which was added to the terminal end of the first strand in step (2); the penultimate nucleotide of the synthesis strand occupies position n+2, wherein position n+2 is the nucleotide position which is occupied by the second nucleotide to be added to the terminal end of the second strand of the scaffold polynucleotide in step (4); the universal nucleotide occupies position n+4+x in the synthesis strand and is paired with a partner nucleotide in the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand; and wherein x is a number of nucleotide positions relative to position n+4 in the direction distal to the complementary ligation end and wherein the number is a whole number from 1 to 10 or more; and
(ii) in step (5) the second strand of the ligated scaffold polynucleotide is cleaved between positions n+2 and n+3.

A method involving a polynucleotide ligation molecule may alternatively be performed as follows:
(A) in step (1) the double-stranded scaffold polynucleotide is provided with a blunt end, with the terminal nucleotide of the second strand paired with the terminal nucleotide of the first strand;
(B) in step (2), in the first polynucleotide ligation molecule the terminal nucleotide of the synthesis strand occupies position n and is paired with the terminal nucleotide of the helper strand, wherein position n is the nucleotide position which is occupied by the first nucleotide to be added to the terminal end of the first strand of the scaffold polynucleotide in step (2); the universal nucleotide is the penultimate nucleotide of the synthesis strand, occupies position n+1 and is paired with the penultimate nucleotide of the helper strand; the terminal nucleotide of the helper strand is a non-ligatable nucleotide; and the complementary ligation end is provided with a blunt end;
(C) in step (3) the first strand of the ligated scaffold polynucleotide is cleaved between positions n and n+1 whereupon the universal nucleotide is removed from the scaffold polynucleotide and the first nucleotide of the first polynucleotide ligation molecule is retained in the scaffold polynucleotide, and whereupon a single base overhang is created in the scaffold polynucleotide with the terminal nucleotide of the first strand overhanging the terminal nucleotide of the second strand;
(D) in step (4), in the second polynucleotide ligation molecule the terminal nucleotide of the synthesis strand occupies position n, wherein position n is the nucleotide position which is occupied by the first nucleotide to be added to the terminal end of the second strand of the scaffold polynucleotide in step (4) and will be paired with the first nucleotide which was added to the terminal end of the first strand in step (2); the universal nucleotide is the penultimate nucleotide of the synthesis strand, occupies position n+1 and is paired with the terminal nucleotide of the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand; and
(E) in step (5) the second strand of the ligated scaffold polynucleotide is cleaved between positions n and n+1 whereupon the universal nucleotide is removed from the scaffold polynucleotide and the first and second nucleotides of the second polynucleotide ligation molecule are retained in the scaffold polynucleotide, and whereupon a blunt end is created in the scaffold polynucleotide with the terminal nucleotide of the second strand paired with the terminal nucleotide of the first strand. Such a method may be performed in accordance with exemplary synthesis methods of the invention versions 2 and 5 as described herein.

A method involving a polynucleotide ligation molecule may alternatively be performed as follows:
(A) in step (1) the double-stranded scaffold polynucleotide is provided with a blunt end, with the terminal nucleotide of the second strand paired with the terminal nucleotide of the first strand;
(B) in step (2), in the first polynucleotide ligation molecule the terminal nucleotide of the synthesis strand occupies position n and is paired with the terminal nucleotide of the helper strand, wherein position n is the nucleotide position which is occupied by the first nucleotide to be added to the terminal end of the first strand of the scaffold polynucleotide in step (2); the universal nucleotide is the penultimate nucleotide of the synthesis strand, occupies position n+1 and is paired with the penultimate nucleotide of the helper strand; the terminal nucleotide of the helper strand is a non-ligatable nucleotide; and the complementary ligation end is provided with a blunt end;
(C) in step (3) the first strand of the ligated scaffold polynucleotide is cleaved between positions n and n+1 whereupon the universal nucleotide is removed from the scaffold polynucleotide and the first nucleotide of the first polynucleotide ligation molecule is retained in the scaffold polynucleotide, and whereupon a single base overhang is created in the scaffold polynucleotide with the terminal nucleotide of the first strand overhanging the terminal nucleotide of the second strand;
(D) in step (4), in the second polynucleotide ligation molecule the terminal nucleotide of the synthesis strand occupies position n, wherein position n is the nucleotide position which is occupied by the first nucleotide to be added to the terminal end of the second strand of the scaffold polynucleotide in step (4) and will be paired with the first nucleotide which was added to the terminal end of the first strand in step (2); the universal nucleotide occupies position n+2 in the synthesis strand and is paired with the penultimate nucleotide of the helper strand; the terminal nucleotide of the helper strand is a non-ligatable nucleotide, occupies position n+1 and is paired with the penultimate nucleotide of the synthesis strand; and the complementary ligation end is provided with a single base overhang, with the terminal nucleotide of the synthesis strand overhanging the terminal nucleotide of the helper strand; and
(E) in step (5) the second strand of the ligated scaffold polynucleotide is cleaved between positions n and n+1 whereupon the universal nucleotide is removed from the scaffold polynucleotide and the first and second nucleotides of the second polynucleotide ligation molecule are retained in the scaffold polynucleotide, and whereupon a blunt end is created in the scaffold polynucleotide with the terminal nucleotide of the second strand paired with the terminal nucleotide of the first strand. Such a method may be performed in accordance with exemplary synthesis method of the invention version 4 as described herein.

In any of the immediately above-described methods which may be performed in accordance with exemplary synthesis method of the invention version 4 as described herein, the methods may comprise variant methods wherein:
(i) in step (4), in the second polynucleotide ligation molecule the terminal nucleotide of the synthesis strand occupies position n, wherein position n is the nucleotide position which is occupied by the first nucleotide to be added to the terminal end of the second strand of the scaffold polynucleotide in step (4) and will be paired with the first nucleotide which was added to the terminal end of the first strand in step (2); the universal nucleotide occupies position n+2+x in the synthesis strand and is paired with a partner nucleotide in the helper strand; the terminal nucleotide of the helper strand is a non-ligatable nucleotide, occupies position n+1 and is paired with the penultimate nucleotide of the synthesis strand; and the complementary ligation end is provided with a single base overhang, with the terminal nucleotide of the synthesis strand overhanging the terminal nucleotide of the helper strand; and wherein x is a number of nucleotide positions relative to position n+2 in the direction distal to the complementary ligation end and wherein the number is a whole number from 1 to 10 or more; and
(ii) in step (5) the second strand of the ligated scaffold polynucleotide is cleaved between positions n and n+1.

A method involving a polynucleotide ligation molecule may alternatively be performed as follows:
(A) in step (1) the double-stranded scaffold polynucleotide is provided with a blunt end, with the terminal nucleotide of the second strand paired with the terminal nucleotide of the first strand;
(B) in step (2), in the first polynucleotide ligation molecule the terminal nucleotide of the synthesis strand occupies position n and is paired with the terminal nucleotide of the helper strand, wherein position n is the nucleotide position which is occupied by the first nucleotide to be added to the terminal end of the first strand of the scaffold polynucleotide in step (2); the universal nucleotide occupies position n+2 in the synthesis strand and is paired with the nucleotide in the helper strand which is next to the penultimate nucleotide of the helper strand in the direction distal to the complementary ligation end; the terminal nucleotide of the helper strand is a non-ligatable nucleotide; and the complementary ligation end is provided with a blunt end;
(C) in step (3) the first strand of the ligated scaffold polynucleotide is cleaved between positions n and n+1 whereupon the universal nucleotide is removed from the scaffold polynucleotide and the first nucleotide of the first polynucleotide ligation molecule is retained in the scaffold polynucleotide, and whereupon a single base overhang is created in the scaffold polynucleotide with the terminal nucleotide of the first strand overhanging the terminal nucleotide of the second strand;
(D) in step (4), in the second polynucleotide ligation molecule the terminal nucleotide of the synthesis strand occupies position n, wherein position n is the nucleotide position which is occupied by the first nucleotide to be added to the terminal end of the second strand of the scaffold polynucleotide in step (4) and will be paired with the first nucleotide which was added to the terminal end of the first strand in step (2); the universal nucleotide is the penultimate nucleotide of the synthesis strand, occupies position n+1 and is paired with the terminal nucleotide of the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand; and
(E) in step (5) the second strand of the ligated scaffold polynucleotide is cleaved between positions n and n+1 whereupon the universal nucleotide is removed from the scaffold polynucleotide and the first and second nucleotides of the second polynucleotide ligation molecule are retained in the scaffold polynucleotide, and whereupon a blunt end is created in the scaffold polynucleotide with the terminal nucleotide of the second strand paired with the terminal nucleotide of the first strand. Such a method may be performed in accordance with exemplary synthesis method of the invention version 6 as described herein.

In any of the immediately above-described methods which may be performed in accordance with exemplary synthesis method of the invention version 6 as described herein, the methods may comprise variant methods wherein:
(i) in step (2), in the first polynucleotide ligation molecule the terminal nucleotide of the synthesis strand occupies position n and is paired with the terminal nucleotide of the helper strand, wherein position n is the nucleotide position which is occupied by the first nucleotide to be added to the terminal end of the first strand of the scaffold polynucleotide in step (2); the universal nucleotide occupies position n+2+x in the synthesis strand and is paired with the nucleotide in the helper strand which is next to the penultimate nucleotide of the helper strand in the direction distal to the complementary ligation end; the terminal nucleotide of the helper strand is a non-ligatable nucleotide; and the complementary ligation end is provided with a blunt end; and wherein x is a number of nucleotide positions relative to position n+2 in the direction distal to the complementary ligation end and wherein the number is a whole number from 1 to 10 or more; and
(ii) in step (3) the first strand of the ligated scaffold polynucleotide is cleaved between positions n and n+1.

In any of the above-described methods which may be performed in accordance with exemplary synthesis method of the invention version 1 as described herein, the methods may comprise variant methods wherein: in step (2) the universal nucleotide is positioned in the synthesis strand of the first polynucleotide ligation molecule at position n+x and wherein in step (3) the ligated first strand of the scaffold polynucleotide is cleaved between positions n+2 and n+1, wherein x is a number of nucleotide positions relative to position n in the direction distal to the complementary ligation end and wherein x is a whole number from 2 to 10 or more.

In any of the above-described methods which may be performed in accordance with exemplary synthesis method of the invention version 1 as described herein, the methods may comprise variant methods wherein: in step (5) the ligated second strand of the scaffold polynucleotide is cleaved between positions n+3 and n+2, wherein x is a number of nucleotide positions relative to position n in the direction distal to the complementary ligation end and wherein x is a whole number from 3 to 10 or more.

In any of the above-described methods which may be performed in accordance with exemplary synthesis method of the invention version 1 as described herein, the methods may comprise variant methods wherein: in step (2) the universal nucleotide is positioned in the synthesis strand of the first polynucleotide ligation molecule at position n+x and wherein in step (3) the ligated first strand of the scaffold polynucleotide is cleaved between positions n+2 and n+1, and wherein in step (4) the universal nucleotide is positioned in the synthesis strand of the second polynucleotide ligation molecule at position n+x and wherein in step (5) the ligated second strand of the scaffold polynucleotide is cleaved between positions n+3 and n+2, wherein x is a number of nucleotide positions relative to position n in the direction distal to the complementary ligation end, and wherein in steps (2) and (3) x is a whole number from 2 to 10 or more and in steps (4) and (5) x is a whole number from 3 to 10 or more.

In any of the above-described methods which may be performed in accordance with exemplary synthesis methods of the invention versions 3 and 5 as described herein, the methods may comprise variant methods wherein: in step (2) the universal nucleotide is positioned in the synthesis strand of the first polynucleotide ligation molecule at a position defined by the formula n+x and wherein in step (3) the ligated first strand of the scaffold polynucleotide is cleaved between positions n+1 and n, wherein x is a number of nucleotide positions relative to position n in the direction distal to the complementary ligation end and wherein x is a whole number from 1 to 10 or more.

In any of the above-described methods which may be performed in accordance with exemplary synthesis methods of the invention versions 3 and 5 as described herein, the methods may comprise variant methods wherein: in step (4) the universal nucleotide is positioned in the synthesis strand of the second polynucleotide ligation molecule at a position defined by the formula n+x and wherein in step (5) the ligated second strand of the scaffold polynucleotide is cleaved between positions n+1 and n, wherein x is a number of nucleotide positions relative to position n in the direction distal to the complementary ligation end and wherein x is a whole number from 1 to 10 or more.

In any of the above-described methods which may be performed in accordance with exemplary synthesis methods of the invention versions 3 and 5 as described herein, the methods may comprise variant methods wherein: in step (2) the universal nucleotide is positioned in the synthesis strand of the first polynucleotide ligation molecule at a position defined by the formula n+x, wherein in step (3) the ligated first strand of the scaffold polynucleotide is cleaved between positions n+1 and n, wherein x is a whole number from 1 to 10 or more; and wherein in step (4) the universal nucleotide is positioned in the synthesis strand of the second polynucleotide ligation molecule at a position defined by the formula n+x and wherein in step (5) the ligated second strand of the scaffold polynucleotide is cleaved between positions n+1 and n wherein x is a whole number from 1 to 10 or more; and wherein in steps (2) and (4) x is a number of nucleotide positions relative to position n in the direction distal to the complementary ligation end.

In any of the above-described methods which may be performed in accordance with exemplary synthesis methods of the invention versions 3 and 5 as described herein, the methods may comprise variant methods wherein: in step (2) the universal nucleotide is positioned in the synthesis strand of the first polynucleotide ligation molecule at a position defined by the formula n+1+x, and wherein in step (3) the ligated first strand of the scaffold polynucleotide is cleaved between positions n+1+x and n+x, wherein x is a number of nucleotide positions relative to position n in the direction distal to the complementary ligation end and wherein x is a whole number from 1 to 10 or more;

In any of the above-described methods which may be performed in accordance with exemplary synthesis methods of the invention versions 3 and 5 as described herein, the methods may comprise variant methods wherein: in step (4) the universal nucleotide is positioned in the synthesis strand of the second polynucleotide ligation molecule at a position defined by the formula n+1+x, and wherein in step (5) the ligated second strand of the scaffold polynucleotide is cleaved between positions n+1+x and n+x, wherein x is a number of nucleotide positions relative to position n in the direction distal to the complementary ligation end and wherein x is a whole number from 1 to 10 or more.

In any of the above-described methods which may be performed in accordance with exemplary synthesis methods of the invention versions 3 and 5 as described herein, the methods may comprise variant methods wherein: in step (2) the universal nucleotide is positioned in the synthesis strand of the first polynucleotide ligation molecule at a position defined by the formula n+1+x, and wherein in step (3) the ligated first strand of the scaffold polynucleotide is cleaved between positions n+1+x and n+x, wherein x is a whole number from 1 to 10 or more; and wherein in step (4) the universal nucleotide is positioned in the synthesis strand of the second polynucleotide ligation molecule at a position defined by the formula n+1+x, and wherein in step (5) the ligated second strand of the scaffold polynucleotide is cleaved between positions n+1+x and n+x, wherein x is a whole number from 1 to 10 or more; and wherein in steps (2) and (4) x is a number of nucleotide positions relative to position n in the direction distal to the complementary ligation end.

In any of the above-described methods which may be performed in accordance with exemplary synthesis methods of the invention versions 3 and 5 as described herein, the methods may comprise variant methods wherein: in step (2) the universal nucleotide is positioned in the synthesis strand of the first polynucleotide ligation molecule at a position defined by the formula n+1+x, wherein in step (3) the ligated first strand of the scaffold polynucleotide is cleaved between positions n+x and n+x−1, wherein x is a number of nucleotide positions relative to position n in the direction distal to the complementary ligation end and wherein x is a whole number from 1 to 10 or more.

In any of the above-described methods which may be performed in accordance with exemplary synthesis methods of the invention versions 3 and 5 as described herein, the methods may comprise variant methods wherein: in step (4) the universal nucleotide is positioned in the synthesis strand of the second polynucleotide ligation molecule at a position defined by the formula n+1+x, wherein in step (5) the ligated second strand of the scaffold polynucleotide is cleaved between positions n+x and n+x−1, wherein x is a number of nucleotide positions relative to position n in the direction distal to the complementary ligation end and wherein x is a whole number from 1 to 10 or more

In any of the above-described methods which may be performed in accordance with exemplary synthesis methods of the invention versions 3 and 5 as described herein, the methods may comprise variant methods wherein: in step (2) the universal nucleotide is positioned in the synthesis strand of the first polynucleotide ligation molecule at a position defined by the formula n+1+x, wherein in step (3) the ligated first strand of the scaffold polynucleotide is cleaved between positions n+x and n+x−1, wherein x is a whole number from 1 to 10 or more; and in step (4) the universal nucleotide is positioned in the synthesis strand of the second polynucleotide ligation molecule at a position defined by the formula n+1+x, wherein in step (5) the ligated second strand of the scaffold polynucleotide is cleaved between positions n+x and n+x−1, wherein x is a whole number from 1 to 10 or more; and wherein in steps (2) and (4) x is a number of nucleotide positions relative to position n in the direction distal to the complementary ligation end.

In any of the above-described methods wherein a strand is cleaved between the position occupied by the universal nucleotide and the position in the same strand occupied by the nucleotide immediately next to the universal nucleotide in the direction distal to the helper strand, in any one, more or all cycles of synthesis cleavage step (3) may comprise a two step cleavage process wherein each cleavage step comprises a first step comprising removing the universal nucleotide thus forming an abasic site, and a second step comprising cleaving the support strand at the abasic site; and/or in any one, more or all cycles of synthesis cleavage step (5) may comprise a two step cleavage process wherein each cleavage step comprises a first step comprising removing the universal nucleotide thus forming an abasic site, and a second step comprising cleaving the support strand at the abasic site. In any of these methods, the first step may be performed with a nucleotide-excising enzyme. The nucleotide-excising enzyme may be a 3-methyladenine DNA glycosylase enzyme. The nucleotide-excising enzyme may be human alkyladenine DNA glycosylase (hAAG) or uracil DNA glycosylase (UDG).

In any of these methods which comprise a two step cleavage process the second step may be performed with a chemical which is a base. The base may be NaOH.

In any of these methods which comprise a two step cleavage process the second step may be performed with an organic chemical having abasic site cleavage activity. The organic chemical may be N,N′-dimethylethylenediamine. The second step may be performed with an enzyme having abasic site lyase activity, optionally wherein the enzyme having abasic site lyase activity is:
(i) AP Endonuclease 1;
(ii) Endonuclease III (Nth); or
(iii) Endonuclease VIII.

Alternatively, in any one, more or all cycles of synthesis cleavage step (3) may comprise a one step cleavage process comprising removing the universal nucleotide with a cleavage enzyme; and/or in any one, more or all cycles of synthesis cleavage step (5) may comprise a one step cleavage process comprising removing the universal nucleotide with a cleavage enzyme; wherein the enzyme is:
(i) Endonuclease III;
(ii) Endonuclease VIII;
(iii) formamidopirimidine DNA glycosylase (Fpg); or
(iv) 8-oxoguanine DNA glycosylase (hOGG1).

In any of the above-described methods wherein a strand is cleaved between the position occupied by the nucleotide immediately next to the universal nucleotide in the direction distal to the helper strand and the position occupied in the same strand by the next nucleotide in the direction distal to the helper strand, in any one, more or all cycles of synthesis cleavage step (3) may comprise cleaving the support strand with an enzyme; and/or in any one, more or all cycles of synthesis cleavage step (5) may comprise cleaving the support strand with an enzyme. The enzyme may be Endonuclease V.

In any of the above-described methods which may be performed in accordance with any one of the exemplary synthesis methods of the invention versions 1, 2, 3 and 4, the method may be performed as follows: in step (1) the terminal nucleotide of the second strand of the scaffold polynucleotide is the 5′ end of the second strand; in step (2) the terminal nucleotide of the synthesis strand of the first polynucleotide ligation molecule is the 5′ end of the synthesis strand; in step (3) the terminal nucleotide of the first strand of the scaffold polynucleotide is the 3′ end of the first strand; and in step (4) the terminal nucleotide of the synthesis strand of the second polynucleotide ligation molecule is the 3′ end of the synthesis strand.

In any of the above-described methods which may be performed in accordance with any one of the exemplary synthesis methods of the invention versions 5 and 6, the method may be performed as follows: in step (1) the terminal nucleotide of the second strand of the scaffold polynucleotide is the 3′ end of the second strand; in step (2) the terminal nucleotide of the synthesis strand of the first polynucleotide ligation molecule is the 3′ end of the synthesis strand; in step (3) the terminal nucleotide of the first strand of the scaffold polynucleotide is the 5′ end of the first strand; and in step (4) the terminal nucleotide of the synthesis strand of the second polynucleotide ligation molecule is the 5′ end of the synthesis strand.

In any of the above-described methods in any one, more or all cycles of synthesis one or more of the nucleotides which are incorporated into one strand of a double-stranded polynucleotide may form a pair with a partner nucleotide(s) at the corresponding position in the opposite strand, and wherein nucleotides of a pair are complementary nucleotides, preferably naturally complementary nucleotides.

In any of the above-described methods in any one, more or all cycles of synthesis, prior to cleavage steps (3) and (5) the helper strand may be removed from the ligated scaffold polynucleotide. The helper strand may be removed from the scaffold polynucleotide by: (i) heating the scaffold polynucleotide to a temperature of about 80° C. to about 95° C. and separating the helper strand from the scaffold polynucleotide, (ii) treating the scaffold polynucleotide with urea solution, such as 8M urea and separating the helper strand from the scaffold polynucleotide, (iii) treating the scaffold polynucleotide with formamide or formamide solution, such as 100% formamide and separating the helper strand from the scaffold polynucleotide, or (iv) contacting the scaffold polynucleotide with a single-stranded polynucleotide molecule which comprises a region of nucleotide sequence which is complementary with the sequence of the helper strand, thereby competitively inhibiting the hybridisation of the helper strand to the scaffold polynucleotide.

In any of the above-described methods, both strands of the synthesised double-stranded polynucleotide may be DNA strands. In such methods incorporated nucleotides may be dNTPs. Alternatively, one strand of the synthesised double-stranded polynucleotide may be a DNA strand and the other strand of the synthesised double-stranded polynucleotide may be an RNA strand. In methods involving an RNA strand, nucleotides incorporated into an RNA strand may be NTPs.

In any of the above-described methods the ligase enzyme may be a T3 DNA ligase or a T4 DNA ligase.

Any of the above-described methods may further comprise further extending the first and/or second strands of the scaffold polynucleotide following cleavage step (3) and/or cleavage step (5) by the action of a polymerase enzyme and/or a transferase enzyme.

In methods involving further extending the first and/or second strands of the scaffold polynucleotide following a cleavage step by the action of a polymerase enzyme, the polymerase enzyme may be a DNA polymerase, optionally a modified DNA polymerase having an enhanced ability to incorporate a dNTP comprising a reversible terminator group compared to an unmodified polymerase. A polymerase enzyme may be a variant of the native DNA polymerase from Thermococcus species 9° N, preferably species 9° N-7. In any of these methods, one or more of the nucleotides incorporated by the polymerase may be dNTPs comprising a reversible terminator group. One or more of the incorporated nucleotides comprising a reversible terminator group may be 3′-O-allyl-dNTPs. One or more of the incorporated nucleotides comprising a reversible terminator group may be 3′-O-azidomethyl-dNTPs.

In methods involving further extending the first and/or second strands of the scaffold polynucleotide following a cleavage step by the action of a polymerase enzyme, the polymerase enzyme may be an RNA polymerase such as T3 or T7 RNA polymerase, optionally a modified RNA polymerase having an enhanced ability to incorporate an NTP comprising a reversible terminator group compared to an unmodified polymerase. In any of these methods, one or more of the nucleotides incorporated by the polymerase may be dNTPs comprising a reversible terminator group. One or more of the incorporated nucleotides comprising a reversible terminator group may be 3′-O-allyl-dNTPs. One or more of the incorporated nucleotides comprising a reversible terminator group may be 3′-O-azidomethyl-dNTPs.

In methods involving further extending the first and/or second strands of the scaffold polynucleotide following a cleavage step by the action of a transferase enzyme the transferase enzyme may have a terminal transferase activity, optionally wherein the enzyme is a terminal nucleotidyl transferase, a terminal deoxynucleotidyl transferase, terminal deoxynucleotidyl transferase (TdT), pol lambda, pol mu or Φ29 DNA polymerase.

In methods involving a reversible terminator group the step of removing the reversible terminator group may be performed with tris(carboxyethyl)phosphine (TCEP).

In any of the above-described methods involving ligating a polynucleotide ligation molecule to a scaffold polynucleotide, in a cycle of synthesis, in a given ligation reaction at the complementary ligation end of the polynucleotide ligation molecule: (a) if the helper strand comprises a non-ligatable terminal nucleotide at the 3′ end of the helper strand, the nucleotide may be a 2′,3′-dideoxynucleotide or a 2′-deoxynucleotide; or (b) if the helper strand comprises a non-ligatable terminal nucleotide at the 5′ end of the helper strand, the nucleotide may lack a phosphate group.

In any of the above-described methods, in any one, more or all cycles of synthesis the first and second strands of the scaffold polynucleotide may be connected by a hairpin loop at the end of the molecule opposite the ligation end.

In any of the above-described methods involving ligating a polynucleotide ligation molecule to a scaffold polynucleotide, in any one, more or all cycles of synthesis in step (2) and/or in step (4) in the polynucleotide ligation molecule the synthesis strand and the helper strand hybridized thereto may be connected by a hairpin loop at the end opposite the complementary ligation end. In such a method in any one, more or all cycles of synthesis:
a) the first and second strands of the scaffold polynucleotide may be connected by a hairpin loop at the end of the molecule opposite the ligation end; and
b) in step (2) and/or in step (4) in the polynucleotide ligation molecule the synthesis strand and the helper strand hybridized thereto may be connected by a hairpin loop at the end opposite the complementary ligation end.

In any of the above-described methods, the first and second strands of the scaffold polynucleotide may be tethered to a common surface. In any such method the first strand and/or the second strand comprises a cleavable linker, wherein the linkers may be cleaved to detach the double-stranded polynucleotide from the surface following synthesis. In any such method the hairpin loop in the scaffold polynucleotide may be tethered to a surface. The hairpin loop may be tethered to a surface via a cleavable linker, wherein the linker may be cleaved to detach the double-stranded polynucleotide from the surface following synthesis. The cleavable linker may be a UV cleavable linker.

In any of the above-described methods involving tethering the first and second strands of the scaffold polynucleotide to a common surface, the surface may be a microparticle. The surface may be a planar surface. The surface may comprise a gel. The surface may comprise a polyacrylamide surface, such as about 2% polyacrylamide, preferably wherein the polyacrylamide surface is coupled to a solid support such as glass.

In any of the above-described methods involving tethering the first and second strands of the scaffold polynucleotide to a common surface, the first and second strands of the scaffold polynucleotide may be tethered to a common surface via one or more covalent bonds. The one or more covalent bonds may be formed between a functional group on the common surface and a functional group on the scaffold molecule, wherein the functional group on the scaffold molecule may be an amine group, a thiol group, a thiophosphate group or a thioamide group. The functional group on the common surface may be a bromoacetyl group, optionally wherein the bromoacetyl group may be provided on a polyacrylamide surface derived using N-(5-bromoacetamidylpentyl) acrylamide (BRAPA).

In any of the above-described methods synthesis cycles may be performed in droplets within a microfluidic system. The microfluidic system may be an electrowetting system. The microfluidic system may be an electrowetting-on-dielectric system (EWOD).

In any of the above-described methods, following synthesis the strands of the double-stranded polynucleotides may be separated to provide a single-stranded polynucleotide having a predefined sequence.

In any of the above-described methods, following synthesis the double-stranded polynucleotide or a region thereof may be amplified, preferably by PCR.

The invention also provides a method of assembling a polynucleotide having a predefined sequence, the method comprising performing the method of any one of the preceding claims to synthesise a first polynucleotide having a predefined sequence and one or more additional polynucleotides having a predefined sequence and joining together the first and one or more additional polynucleotides.

In any such method the first polynucleotide and the one or more additional polynucleotides may be double-stranded. In any such method the first polynucleotide and the one or more additional polynucleotides may be single-stranded. In any of these methods the first polynucleotide and the one or more additional polynucleotides may be cleaved to create compatible termini and joined together, preferably by ligation. The first polynucleotide and the one or more additional polynucleotides may be cleaved by a restriction enzyme at a cleavage site.

In any of these methods of assembling a polynucleotide having a predefined sequence the synthesis and/or assembly steps may be performed in droplets within a microfluidic system. In any such method, the assembly steps may comprise providing a first droplet comprising a first synthesised polynucleotide having a predefined sequence and a second droplet or a plurality of further droplets each comprising an additional one or more synthesised polynucleotides having a predefined sequence, wherein the droplets are brought in contact with each other and wherein the synthesised polynucleotides are joined together thereby assembling a polynucleotide comprising the first and additional one or more polynucleotides. In any such method, the synthesis steps may be performed by providing a plurality of droplets each droplet comprising reaction reagents corresponding to a step of the synthesis cycle, and sequentially delivering the droplets to the scaffold polynucleotide in accordance with the steps of the synthesis cycles. In any such method, following delivery of a droplet and prior to the delivery of a next droplet, a washing step may be carried out to remove excess reaction reagents. In any such method, the microfluidic system may be an electrowetting system. In any such method, the microfluidic system may be an electrowetting-on-dielectric system (EWOD). In any such method, the synthesis and assembly steps may be performed within the same system.

In a related aspect, the invention further provides an in vitro method of extending a double-stranded polynucleotide to synthesise a double-stranded polynucleotide having a predefined sequence, the method comprising one or more cycles of synthesis wherein in each cycle of synthesis a universal nucleotide and one or more nucleotides of the predefined sequence are added to a first strand of a double-stranded scaffold polynucleotide in a first extension/ligation reaction, the first strand of the ligated scaffold polynucleotide is cleaved in a first cleavage step at a cleavage site defined by a sequence comprising the universal nucleotide, wherein upon cleavage the universal nucleotide is released from the scaffold polynucleotide and the one or more nucleotides of the predefined sequence are retained in the first strand of the scaffold polynucleotide, a further universal nucleotide and one or more nucleotides of the predefined sequence are added to the second strand of the double-stranded scaffold polynucleotide in a second extension/ligation reaction; and the second strand of the ligated scaffold polynucleotide is cleaved in a second cleavage step at a cleavage site defined by a sequence comprising the further universal nucleotide, wherein upon cleavage the further universal nucleotide is released from the scaffold polynucleotide and the one or more nucleotides of the predefined sequence are retained in the second strand of the scaffold polynucleotide.

Such an in vitro method of extending a double-stranded polynucleotide to synthesise a double-stranded polynucleotide having a predefined sequence may be implemented using any of the specific methods defined and described above and herein.

In a related aspect, the invention further provides the use of a universal nucleotide in an in vitro method of extending a double-stranded polynucleotide to synthesise a double-stranded polynucleotide having a predefined sequence, wherein in a cycle of synthesis a universal nucleotide and one or more nucleotides of the predefined sequence are added to a first strand of a double-stranded scaffold polynucleotide in a first extension/ligation reaction, the first strand of the ligated scaffold polynucleotide is cleaved in a first cleavage step at a cleavage site defined by a sequence comprising the universal nucleotide, wherein upon cleavage the universal nucleotide is released from the scaffold polynucleotide and the one or more nucleotides of the predefined sequence are retained in the first strand of the scaffold polynucleotide, a further universal nucleotide and one or more nucleotides of the predefined sequence are added to the second strand of the double-stranded scaffold polynucleotide in a second extension/ligation reaction; and the second strand of the ligated scaffold polynucleotide is cleaved in a second cleavage step at a cleavage site defined by a sequence comprising the further universal nucleotide, wherein upon cleavage the further universal nucleotide is released from the scaffold polynucleotide and the one or more nucleotides of the predefined sequence are retained in the second strand of the scaffold polynucleotide.

Such use of a universal nucleotide in an in vitro method of synthesising a double-stranded polynucleotide having a predefined sequence may be implemented using any of the specific methods defined and described above and herein.

In a related aspect, the invention further provides an in vitro method of extending with one or more predefined nucleotides each strand of a double-stranded polynucleotide molecule at the same terminal end, the method comprising providing a double-stranded scaffold polynucleotide comprising a first strand and a second strand hybridized thereto, adding a universal nucleotide and one or more nucleotides of the predefined sequence to a first strand of the double-stranded scaffold polynucleotide in a first extension/ligation reaction, cleaving the first strand of the ligated scaffold polynucleotide in a first cleavage step at a cleavage site defined by a sequence comprising the universal nucleotide, wherein upon cleavage the universal nucleotide is released from the scaffold polynucleotide and the one or more nucleotides of the predefined sequence are retained in the first strand of the scaffold polynucleotide, adding a further universal nucleotide and one or more nucleotides of the predefined sequence to the second strand of the double-stranded scaffold polynucleotide in a second extension/ligation reaction; and cleaving the second strand of the ligated scaffold polynucleotide in a second cleavage step at a cleavage site defined by a sequence comprising the further universal nucleotide, wherein upon cleavage the further universal nucleotide is released from the scaffold polynucleotide and the one or more nucleotides of the predefined sequence are retained in the second strand of the scaffold polynucleotide.

Such an in vitro method of extending with one or more predefined nucleotides each strand of a double-stranded polynucleotide molecule at the same terminal end may be implemented using any of the specific methods defined and described above and herein.

In a related aspect, the invention further provides an in vitro method of ligating two polynucleotide ligation molecules each comprising a universal nucleotide to a double-stranded scaffold polynucleotide during a cycle of extending each strand of the double-stranded scaffold polynucleotide with one or more predefined nucleotides at the same terminal end, the method comprising: providing a double-stranded scaffold polynucleotide comprising a first strand and a second strand hybridized thereto, ligating a first polynucleotide ligation molecule comprising a universal nucleotide and one or more nucleotides of the predefined sequence to a first strand of the double-stranded scaffold polynucleotide in a first extension/ligation reaction, cleaving the first strand of the ligated scaffold polynucleotide in a first cleavage step at a cleavage site defined by a sequence comprising the universal nucleotide, wherein upon cleavage the first polynucleotide ligation molecule and the universal nucleotide is released from the scaffold polynucleotide and the one or more nucleotides of the predefined sequence are retained in the first strand of the scaffold polynucleotide, ligating a second polynucleotide ligation molecule comprising a further universal nucleotide and one or more nucleotides of the predefined sequence to the second strand of the double-stranded scaffold polynucleotide in a second extension/ligation reaction; and cleaving the second strand of the ligated scaffold polynucleotide in a second cleavage step at a cleavage site defined by a sequence comprising the further universal nucleotide, wherein upon cleavage the second polynucleotide ligation molecule and the further universal nucleotide is released from the scaffold polynucleotide and the one or more nucleotides of the predefined sequence are retained in the second strand of the scaffold polynucleotide.

Such an in vitro method of ligating two polynucleotide ligation molecules each comprising a universal nucleotide to a double-stranded scaffold polynucleotide during a cycle of extending each strand of the double-stranded scaffold polynucleotide with one or more predefined nucleotides at the same terminal end may be implemented using any of the specific methods defined and described above and herein.

In any of the in vitro methods for synthesising a double-stranded polynucleotide having a predefined sequence as described above and herein, the universal nucleotide may be inosine, or an analogue, variant or derivative thereof. The partner nucleotide for the universal nucleotide in the helper strand may be cytosine. The universal nucleotide may be inosine, or an analogue, variant or derivative thereof and the partner nucleotide in the helper strand may be cytosine.

The invention also provides a method of storing data in a polynucleotide molecule, the method comprising: (a) performing a first series of extension reactions by extending one strand of a double-stranded polynucleotide and then extending the opposite strand by a method according to any one of the in vitro methods for synthesising a double-stranded polynucleotide having a predefined sequence as described above and herein, thereby extending the polynucleotide molecule by one or more pairs of nucleotides to generate a first nucleotide sequence; and (b) performing one or more further series of extension reactions by further extending one strand of the double-stranded polynucleotide and then further extending the opposite strand by a method according to any one of the in vitro methods for synthesising a double-stranded polynucleotide having a predefined sequence as described above and herein, thereby extending the polynucleotide molecule by one or more further pairs of nucleotides, to generate a second or further nucleotide sequence in the polynucleotide, wherein generated sequences are indicative of information encoded into the extended polynucleotide molecule.

The invention also provides a method of storing data in bit form in a polynucleotide molecule, the method comprising: (a) performing a first series of extension reactions by extending one strand of a double-stranded polynucleotide and then extending the opposite strand by a method according to any one of the in vitro methods for synthesising a double-stranded polynucleotide having a predefined sequence as described above and herein, thereby extending the polynucleotide molecule by one or more pairs of nucleotides to generate a first nucleotide sequence in the polynucleotide molecule indicative of a first bit of information; and (b) performing one or more further series of extension reactions by further extending one strand of the double-stranded polynucleotide and then further extending the opposite strand by a method according to any one of the in vitro methods for synthesising a double-stranded polynucleotide having a predefined sequence as described above and herein, thereby extending the polynucleotide molecule by one or more further pairs of nucleotides to generate further nucleotide sequences in the polynucleotide molecule indicative of one or more further bits of information.

The invention also provides a method of storing data in digital form in a polynucleotide molecule, the method comprising: (a) performing a first series of extension reactions by extending one strand of a double-stranded polynucleotide and then extending the opposite strand by a method according to any one of the in vitro methods for synthesising a double-stranded polynucleotide having a predefined sequence as described above and herein, thereby extending the polynucleotide molecule by one or more pairs of nucleotides to generate a first nucleotide sequence in the polynucleotide molecule indicative of the “0” or “1” state of a bit of digital information; and (b) performing one or more further series of extension reactions by further extending one strand of the double-stranded polynucleotide and then further extending the opposite strand by a method according to any one of the in vitro methods for synthesising a double-stranded polynucleotide having a predefined sequence as described above and herein, thereby extending the polynucleotide molecule by one or more further pairs of nucleotides to generate a second nucleotide sequence in the polynucleotide molecule indicative of the opposite state of the bit to that generated in step (a). Any such method may comprise repeating steps (a) and (b) multiple times to generate nucleotide sequences indicative of multiple bits of digital information.

The invention also provides a method of making a polynucleotide microarray, wherein the microarray comprises a plurality of reaction areas, each area comprising one or more polynucleotides having a predefined sequence, the method comprising:
a) providing a surface comprising a plurality of reaction areas, each area comprising one or more double-stranded anchor or scaffold polynucleotides, and
b) performing cycles of synthesis according to the method of any one of the in vitro methods for synthesising a double-stranded polynucleotide having a predefined sequence as described above and herein at each reaction area, thereby synthesising at each area one or more double-stranded polynucleotides having a predefined sequence.

In any such method, following synthesis the strands of the double-stranded polynucleotides may be separated, whereupon each area of the microarray comprises one or more single-stranded polynucleotides having a predefined sequence.

The invention also provides a polynucleotide synthesis system for carrying out the method according to any one of the in vitro methods for synthesising a double-stranded polynucleotide having a predefined sequence as described above and herein, the system comprising: (a) an array of reaction areas, wherein each reaction area comprises at least one scaffold polynucleotide; and (b) means for the delivery of the reaction reagents to the reaction areas; and optionally, (c) means to cleave the synthesised double-stranded polynucleotide from the scaffold polynucleotide. Any such system may further comprise means for providing the reaction reagents in droplets and means for delivering the droplets to the scaffold polynucleotide in accordance with the synthesis cycles.

The invention also provides a kit for use with any one of the systems described herein and for carrying out the method according to any one of the in vitro methods for synthesising a double-stranded polynucleotide having a predefined sequence as described above and herein, the kit comprising volumes of reaction reagents corresponding to the steps of the synthesis cycles.

### DESCRIPTION OF THE FIGURES

Relevant Figures presented herein and described below show some or all of the steps of a cycle of synthesis using methods, including methods of the invention, as well as means for performing aspects of the methods, such as oligonucleotides, surfaces, surface attachment chemistries, linkers etc. These Figures as well as all descriptions thereof and all associated methods, reagents and protocols are presented for illustration only and are not to be construed as limiting.

Relevant Figures, such as e.g. FIGS. 11, 12, 13, 14, 15, 18a, 19a, 20a etc. show some or all of the steps of a cycle of synthesis including incorporation of a nucleotide (e.g., a nucleotide comprising a reversible terminator group), cleavage (e.g., cleaving the scaffold polynucleotide into a first portion and a second portion, wherein the first portion comprises an universal nucleotide, and the second portion comprises the incorporated nucleotide), ligation (e.g., ligating to the second portion of the cleaved scaffold polynucleotide comprising the incorporated nucleotide, a polynucleotide construct comprising a single-stranded portion, wherein the single-stranded portion comprises a partner nucleotide that is complementary to the incorporated nucleotide) and deprotection (e.g., removing the reversible terminator group from the incorporated nucleotide). These methods are provided for illustrative support only and are not within the scope of the claimed invention. Method schemes shown in FIGS. 1 to 10, as well as in FIGS. 57, 60, 61, and 64 to 69 are methods of the invention.

FIG. 1. Scheme of Exemplary Method Version 1 of the Invention.

FIG. 1A is a legend for the various structures depicted in FIGS. 1B and 1n FIGS. 2 to 10.

FIG. 1B is a scheme showing a first synthesis cycle according to exemplary method version 1 of the invention.

The method comprises cycles of provision of a scaffold polynucleotide, ligation of a first polynucleotide ligation molecule to the scaffold polynucleotide, a first cleavage step leading to the incorporation of two nucleotides into the first strand of the scaffold polynucleotide, ligation of a second polynucleotide ligation molecule to the scaffold polynucleotide, and a second cleavage step leading to the incorporation of two nucleotides into the second strand of the scaffold polynucleotide.

The scheme shows the provision of a scaffold polynucleotide (101, 106). One end of the scaffold polynucleotide is to be extended by the incorporation of nucleotides of the predefined sequence into both strands (shown as the upper end of the scaffold polynucleotide). The opposite end of the scaffold polynucleotide (shown as the lower end, labelled 3′ and 5′) is shown not to be extended. The scaffold polynucleotide comprises a first strand (dotted lines) and a second strand (dotted-and-dashed lines) hybridised thereto. The end of the scaffold polynucleotide to be extended is shown with a single-base overhang. The terminal nucleotide of the second strand at the end of the scaffold polynucleotide to be extended is depicted as “A” (adenosine) and overhangs the terminal nucleotide of the first strand. The terminal nucleotide of the second strand is a ligatable nucleotide. The terminal nucleotide of the first strand, which is also a ligatable nucleotide, is paired with the penultimate nucleotide of the second strand in a nucleotide pair. Both nucleotides of the pair are depicted as “X”. These two nucleotides can be any two nucleotides or analogs or derivatives thereof, and are not limited to being a naturally complementary pair of nucleotides. The overhanging terminal nucleotide of the second strand can be any nucleotide or analog or derivative thereof.

The scheme shows the provision of a first polynucleotide ligation molecule (102, 107; structure in the top right of the Figure). The polynucleotide ligation molecule comprises a helper strand (dashed line), a synthesis strand (solid line) hybridised thereto and a complementary ligation end. The terminal nucleotide of the synthesis strand of the complementary ligation end is a ligatable first nucleotide of the predefined sequence to be incorporated into the first strand, is depicted as “T” (thymine) and overhangs the terminal nucleotide of the helper strand of the complementary ligation end in a single-nucleotide overhang. The terminal nucleotide of the helper strand is depicted as a non-ligatable “C” (cytosine) and is paired with the penultimate nucleotide of the synthesis strand which is depicted as “G” (guanine). The complementary ligation end comprises a universal nucleotide (depicted as “Un”) in the synthesis strand and which is paired with a partner nucleotide in the helper strand (depicted as “X”). T, C, G and X are depicted purely for illustration and can be any nucleotides or analogs or derivatives thereof. It is not necessary for paired nucleotides to comprise naturally complementary nucleotides.

The scheme shows the ligation of the synthesis strand of the first polynucleotide ligation molecule (102, 107) to the first strand of the scaffold polynucleotide and the creation of single-stranded break (“nick”) between the helper strand of the first polynucleotide ligation molecule and the second strand.

The scheme shows a first cleavage step (103, 108) comprising cleaving the first/synthesis strand (jagged arrowhead) at a cleavage site defined by a sequence comprising the universal nucleotide. Cleavage releases the first polynucleotide ligation molecule comprising the universal nucleotide and leads to the retention in the scaffold polynucleotide of the two nucleotides (T and G) derived from the first polynucleotide ligation molecule. In the first cleavage step the first/synthesis strand is cleaved between the positions occupied by the universal nucleotide and the nucleotide which occupies the next nucleotide position in the first/synthesis strand in the direction distal to the helper strand. Cleavage leaves in place a single-base overhang at the cleaved end of the scaffold polynucleotide with the terminal ligatable nucleotide of the first strand overhanging the terminal ligatable nucleotide of the second strand.

The scheme shows the provision of a second polynucleotide ligation molecule (104, 109). The polynucleotide ligation molecule comprises a helper strand (dashed line), a synthesis strand (solid line) hybridised thereto and a complementary ligation end. The terminal nucleotide of the synthesis strand of the complementary ligation end is a first nucleotide of the predefined sequence to be incorporated into the second strand, is depicted as a ligatable “C” (cytosine) and overhangs the terminal nucleotide of the helper strand of the complementary ligation end in a single-nucleotide overhang. The terminal nucleotide of the helper strand is depicted as a non-ligatable “A” (adenine) and is paired with the penultimate nucleotide of the synthesis strand which is depicted as “T” (thymine). The complementary ligation end comprises a universal nucleotide (depicted as “Un”) in the synthesis strand and which is paired with a partner nucleotide at the penultimate position in the helper strand (depicted as “X”). C, A, T and X are depicted purely for illustration and can be any nucleotides or analogs or derivatives thereof. It is not necessary for paired nucleotides to comprise naturally complementary nucleotides.

The scheme shows the ligation of the synthesis strand of the second polynucleotide ligation molecule (104, 109) to the second strand of the scaffold polynucleotide and the creation of single-stranded break (“nick”) between the helper strand of the second polynucleotide ligation molecule and the first strand.

The scheme shows a second cleavage step (105, 110) comprising cleaving the second/synthesis strand (jagged arrowhead) at a cleavage site defined by a sequence comprising the universal nucleotide. Cleavage releases the second polynucleotide ligation molecule comprising the universal nucleotide and leads to the retention in the scaffold polynucleotide of the two nucleotides (C and T) derived from the second polynucleotide ligation molecule. In the second cleavage step the second/synthesis strand is cleaved between the position occupied by the universal nucleotide and the nucleotide which occupies the next nucleotide position in the second/synthesis strand in the direction distal to the helper strand. Cleavage leaves in place a single-base overhang at the cleaved end of the scaffold polynucleotide with the terminal ligatable nucleotide of the second strand overhanging the terminal ligatable nucleotide of the first strand.

FIG. 2. Scheme of Exemplary Method Version 2 of the Invention.

Scheme showing a first synthesis cycle according to exemplary method version 2 of the invention.

The method comprises cycles of provision of a scaffold polynucleotide, ligation of a first polynucleotide ligation molecule to the scaffold polynucleotide, a first cleavage step leading to the incorporation of two nucleotides into the first strand of the scaffold polynucleotide, ligation of a second polynucleotide ligation molecule to the scaffold polynucleotide, and a second cleavage step leading to the incorporation of two nucleotides into the second strand of the scaffold polynucleotide.

The scheme shows the provision of a scaffold polynucleotide (201, 206). One end of the scaffold polynucleotide is to be extended by the incorporation of nucleotides of the predefined sequence into both strands (shown as the upper end of the scaffold polynucleotide). The opposite end of the scaffold polynucleotide (shown as the lower end, labelled 3′ and 5′) is shown not to be extended. The scaffold polynucleotide comprises a first strand (dotted lines) and a second strand (dotted-and-dashed lines) hybridised thereto. The end of the scaffold polynucleotide to be extended is shown with a single-base overhang. The terminal nucleotide of the second strand at the end of the scaffold polynucleotide to be extended is depicted as “A” (adenosine) and overhangs the terminal nucleotide of the first strand. The terminal nucleotide of the second strand is a ligatable nucleotide. The terminal nucleotide of the first strand, which is also a ligatable nucleotide, is paired with the penultimate nucleotide of the second strand in a nucleotide pair. Both nucleotides of the pair are depicted as “X”. These two nucleotides can be any two nucleotides or analogs or derivatives thereof, and are not limited to being a naturally complementary pair of nucleotides. The overhanging terminal nucleotide of the second strand can be any nucleotide or analog or derivative thereof.

The scheme shows the provision of a first polynucleotide ligation molecule (202, 207; structure in the top right of the Figure). The polynucleotide ligation molecule comprises a helper strand (dashed line), a synthesis strand (solid line) hybridised thereto and a complementary ligation end. The terminal nucleotide of the synthesis strand of the complementary ligation end is a ligatable first nucleotide of the predefined sequence to be incorporated into the first strand, is depicted as “T” (thymine) and overhangs the terminal nucleotide of the helper strand of the complementary ligation end in a single-nucleotide overhang. The terminal nucleotide of the helper strand is depicted as a non-ligatable “C” (cytosine) and is paired with the penultimate nucleotide of the synthesis strand which is depicted as “G” (guanine). The complementary ligation end comprises a universal nucleotide (depicted as “Un”) in the synthesis strand and which is paired with a partner nucleotide in the helper strand (depicted as “X”). T, C, G and X are depicted purely for illustration and can be any nucleotides or analogs or derivatives thereof. It is not necessary for paired nucleotides to comprise naturally complementary nucleotides.

The scheme shows the ligation of the synthesis strand of the first polynucleotide ligation molecule (202, 207) to the first strand of the scaffold polynucleotide and the creation of single-stranded break (“nick”) between the helper strand of the first polynucleotide ligation molecule and the second strand.

The scheme shows a first cleavage step (203, 208) comprising cleaving the first/synthesis strand (jagged arrowhead) at a cleavage site defined by a sequence comprising the universal nucleotide. Cleavage releases the first polynucleotide ligation molecule comprising the universal nucleotide and leads to the retention in the scaffold polynucleotide of the two nucleotides (T and G) derived from the first polynucleotide ligation molecule. In the first cleavage step the first/synthesis strand is cleaved between the positions occupied by the universal nucleotide and the nucleotide which occupies the next nucleotide position in the first/synthesis strand in the direction distal to the helper strand. Cleavage leaves in place a single-base overhang at the cleaved end of the scaffold polynucleotide with the terminal ligatable nucleotide of the first strand overhanging the terminal ligatable nucleotide of the second strand.

The scheme shows the provision of a second polynucleotide ligation molecule (204, 209). The polynucleotide ligation molecule comprises a helper strand (dashed line), a synthesis strand (solid line) hybridised thereto and a complementary ligation end. The terminal nucleotide of the synthesis strand of the complementary ligation end is a first nucleotide of the predefined sequence to be incorporated into the second strand, is depicted as a ligatable “C” (cytosine) and overhangs the terminal nucleotide of the helper strand of the complementary ligation end in a single-nucleotide overhang. The terminal nucleotide of the helper strand is depicted as a non-ligatable “A” (adenine) and is paired with the penultimate nucleotide of the synthesis strand which is depicted as “T” (thymine). The complementary ligation end comprises a universal nucleotide (depicted as “Un”) in the synthesis strand and which is paired with a partner nucleotide in the helper strand (depicted as “X”), wherein X occupies the position immediately next to the penultimate nucleotide in the helper strand in the direction distal to the complementary ligation end. C, A, T and X are depicted purely for illustration and can be any nucleotides or analogs or derivatives thereof. It is not necessary for paired nucleotides to comprise naturally complementary nucleotides.

The scheme shows the ligation of the synthesis strand of the second polynucleotide ligation molecule (204, 209) to the second strand of the scaffold polynucleotide and the creation of single-stranded break (“nick”) between the helper strand of the second polynucleotide ligation molecule and the first strand.

The scheme shows a second cleavage step (205, 210) comprising cleaving the second/synthesis strand (jagged arrowhead) at a cleavage site defined by a sequence comprising the universal nucleotide. Cleavage releases the second polynucleotide ligation molecule comprising the universal nucleotide and leads to the retention in the scaffold polynucleotide of the two nucleotides (C and T) derived from the second polynucleotide ligation molecule. In the second cleavage step the second/synthesis strand is cleaved between the positions occupied by the nucleotides which are the first and second nucleotides immediately next to the universal nucleotide in the second/synthesis strand in the direction distal to the helper strand. Cleavage leaves in place a single-base overhang at the cleaved end of the scaffold polynucleotide with the terminal ligatable nucleotide of the second strand overhanging the terminal ligatable nucleotide of the first strand.

FIG. 3. Scheme of Exemplary Method Version 3 of the Invention.

Scheme showing a first synthesis cycle according to exemplary method version 3 of the invention.

The method comprises cycles of provision of a scaffold polynucleotide, ligation of a first polynucleotide ligation molecule to the scaffold polynucleotide, a first cleavage step leading to the incorporation of one nucleotide into the first strand of the scaffold polynucleotide, ligation of a second polynucleotide ligation molecule to the scaffold polynucleotide, and a second cleavage step leading to the incorporation of one nucleotide into the second strand of the scaffold polynucleotide.

The scheme shows the provision of a scaffold polynucleotide (301, 306). One end of the scaffold polynucleotide is to be extended by the incorporation of a nucleotide of the predefined sequence into both strands (shown as the upper end of the scaffold polynucleotide). The opposite end of the scaffold polynucleotide (shown as the lower end, labelled 3′ and 5′) is shown not to be extended. The scaffold polynucleotide comprises a first strand (dotted lines) and a second strand (dotted-and-dashed lines) hybridised thereto. The end of the scaffold polynucleotide to be extended is shown with a blunt end. The terminal nucleotide of the first strand is depicted as “X” and is paired with the terminal nucleotide of the second strand, also depicted as “X”. The terminal nucleotides of the first and second strands at the end of the scaffold polynucleotide to be extended are ligatable nucleotides. These two nucleotides can be any two nucleotides or analogs or derivatives thereof, and are not limited to being a naturally complementary pair of nucleotides.

The scheme shows the provision of a first polynucleotide ligation molecule (302, 307; structure in the top right of the Figure). The polynucleotide ligation molecule comprises a helper strand (dashed line), a synthesis strand (solid line) hybridised thereto and a complementary ligation end. The terminal nucleotide of the synthesis strand of the complementary ligation end is a ligatable first nucleotide of the predefined sequence to be incorporated into the first strand, is depicted as “T” (thymine) and is paired with the terminal nucleotide of the helper strand of the complementary ligation end in a bunt end. The terminal nucleotide of the helper strand is depicted as a non-ligatable “A” (adenine). The complementary ligation end comprises a universal nucleotide (depicted as “Un”) in the synthesis strand and which is paired with a partner nucleotide in the helper strand (depicted as “X”) and which is the penultimate nucleotide in the helper strand. T, A and X are depicted purely for illustration and can be any nucleotides or analogs or derivatives thereof. It is not necessary for paired nucleotides to comprise naturally complementary nucleotides.

The scheme shows the ligation of the synthesis strand of the first polynucleotide ligation molecule (302, 307) to the first strand of the scaffold polynucleotide and the creation of single-stranded break (“nick”) between the helper strand of the first polynucleotide ligation molecule and the second strand.

The scheme shows a first cleavage step (303, 308) comprising cleaving the first/synthesis strand (jagged arrowhead) at a cleavage site defined by a sequence comprising the universal nucleotide. Cleavage releases the first polynucleotide ligation molecule comprising the universal nucleotide and leads to the retention in the scaffold polynucleotide of the thymine nucleotide derived from the first polynucleotide ligation molecule. In the first cleavage step the first/synthesis strand is cleaved between the positions occupied by the universal nucleotide and the nucleotide which occupies the next nucleotide position in the first/synthesis strand in the direction distal to the helper strand. Cleavage leaves in place a single-base overhang at the cleaved end of the scaffold polynucleotide with the terminal ligatable nucleotide of the first strand overhanging the terminal ligatable nucleotide of the second strand.

The scheme shows the provision of a second polynucleotide ligation molecule (304, 309). The polynucleotide ligation molecule comprises a helper strand (dashed line), a synthesis strand (solid line) hybridised thereto and a complementary ligation end. The terminal nucleotide of the synthesis strand of the complementary ligation end is a first nucleotide of the predefined sequence to be incorporated into the second strand, is depicted as a ligatable “A” (adenine) and overhangs the terminal nucleotide of the helper strand of the complementary ligation end in a single-nucleotide overhang. The terminal nucleotide of the helper strand is depicted as a non-ligatable nucleotide “X” and is paired with the universal nucleotide (depicted as “Un”), which is the penultimate nucleotide of the synthesis strand at the complementary ligation end. A and X are depicted purely for illustration and can be any nucleotides or analogs or derivatives thereof. It is not necessary for paired nucleotides to comprise naturally complementary nucleotides.

The scheme shows the ligation of the synthesis strand of the second polynucleotide ligation molecule (304, 309) to the second strand of the scaffold polynucleotide and the creation of single-stranded break (“nick”) between the helper strand of the second polynucleotide ligation molecule and the first strand.

The scheme shows a second cleavage step (305, 310) comprising cleaving the second/synthesis strand (jagged arrowhead) at a cleavage site defined by a sequence comprising the universal nucleotide. Cleavage releases the second polynucleotide ligation molecule comprising the universal nucleotide and leads to the retention in the scaffold polynucleotide of the adenine nucleotide (A) derived from the second polynucleotide ligation molecule. In the second cleavage step the second/synthesis strand is cleaved between the position occupied by the universal nucleotide and the nucleotide which occupies the next nucleotide position in the second/synthesis strand in the direction distal to the helper strand. Cleavage leaves in place a blunt-ended cleaved end of the scaffold polynucleotide with the terminal ligatable nucleotide of the second strand paired with the terminal ligatable nucleotide of the first strand.

FIG. 4. Scheme of Exemplary Method Version 4 of the Invention.

Scheme showing a first synthesis cycle according to exemplary method version 4 of the invention.

The method comprises cycles of provision of a scaffold polynucleotide, ligation of a first polynucleotide ligation molecule to the scaffold polynucleotide, a first cleavage step leading to the incorporation of one nucleotide into the first strand of the scaffold polynucleotide, ligation of a second polynucleotide ligation molecule to the scaffold polynucleotide, and a second cleavage step leading to the incorporation of one nucleotide into the second strand of the scaffold polynucleotide.

The scheme shows the provision of a scaffold polynucleotide (401, 406). One end of the scaffold polynucleotide is to be extended by the incorporation of a nucleotide of the predefined sequence into both strands (shown as the upper end of the scaffold polynucleotide). The opposite end of the scaffold polynucleotide (shown as the lower end, labelled 3′ and 5′) is shown not to be extended. The scaffold polynucleotide comprises a first strand (dotted lines) and a second strand (dotted-and-dashed lines) hybridised thereto. The end of the scaffold polynucleotide to be extended is shown with a blunt end. The terminal nucleotide of the first strand is depicted as “X” and is paired with the terminal nucleotide of the second strand, also depicted as “X”. The terminal nucleotides of the first and second strands at the end of the scaffold polynucleotide to be extended are ligatable nucleotides. These two nucleotides can be any two nucleotides or analogs or derivatives thereof, and are not limited to being a naturally complementary pair of nucleotides.

The scheme shows the provision of a first polynucleotide ligation molecule (402, 407; structure in the top right of the Figure). The polynucleotide ligation molecule comprises a helper strand (dashed line), a synthesis strand (solid line) hybridised thereto and a complementary ligation end. The terminal nucleotide of the synthesis strand of the complementary ligation end is a ligatable first nucleotide of the predefined sequence to be incorporated into the first strand, is depicted as “T” (thymine) and is paired with the terminal nucleotide of the helper strand of the complementary ligation end in a bunt end. The terminal nucleotide of the helper strand is depicted as a non-ligatable “A” (adenine). The complementary ligation end comprises a universal nucleotide (depicted as “Un”) in the synthesis strand and which is paired with a partner nucleotide in the helper strand (depicted as “X”) and which is the penultimate nucleotide in the helper strand. T, A and X are depicted purely for illustration and can be any nucleotides or analogs or derivatives thereof. It is not necessary for paired nucleotides to comprise naturally complementary nucleotides.

The scheme shows the ligation of the synthesis strand of the first polynucleotide ligation molecule (402, 407) to the first strand of the scaffold polynucleotide and the creation of single-stranded break (“nick”) between the helper strand of the first polynucleotide ligation molecule and the second strand.

The scheme shows a first cleavage step (403, 408) comprising cleaving the first/synthesis strand (jagged arrowhead) at a cleavage site defined by a sequence comprising the universal nucleotide. Cleavage releases the first polynucleotide ligation molecule comprising the universal nucleotide and leads to the retention in the scaffold polynucleotide of the thymine nucleotide derived from the first polynucleotide ligation molecule. In the first cleavage step the first/synthesis strand is cleaved between the positions occupied by the universal nucleotide and the nucleotide which occupies the next nucleotide position in the first/synthesis strand in the direction distal to the helper strand. Cleavage leaves in place a single-base overhang at the cleaved end of the scaffold polynucleotide with the terminal ligatable nucleotide of the first strand overhanging the terminal ligatable nucleotide of the second strand.

The scheme shows the provision of a second polynucleotide ligation molecule (404, 409). The polynucleotide ligation molecule comprises a helper strand (dashed line), a synthesis strand (solid line) hybridised thereto and a complementary ligation end. The terminal nucleotide of the synthesis strand of the complementary ligation end is a first nucleotide of the predefined sequence to be incorporated into the second strand, is depicted as a ligatable “A” (adenine) and overhangs the terminal nucleotide of the helper strand of the complementary ligation end in a single-nucleotide overhang. The terminal nucleotide of the helper strand is depicted as a non-ligatable nucleotide “X” and is paired with the penultimate nucleotide of the synthesis strand, also depicted as “X”. The penultimate nucleotide of the helper strand, also depicted as “X”, is paired with the universal nucleotide (depicted as “Un”). A and X are depicted purely for illustration and can be any nucleotides or analogs or derivatives thereof. It is not necessary for paired nucleotides to comprise naturally complementary nucleotides.

The scheme shows the ligation of the synthesis strand of the second polynucleotide ligation molecule (404, 409) to the second strand of the scaffold polynucleotide and the creation of single-stranded break (“nick”) between the helper strand of the second polynucleotide ligation molecule and the first strand.

The scheme shows a second cleavage step (405, 410) comprising cleaving the second/synthesis strand (jagged arrowhead) at a cleavage site defined by a sequence comprising the universal nucleotide. Cleavage releases the second polynucleotide ligation molecule comprising the universal nucleotide and leads to the retention in the scaffold polynucleotide of the adenine nucleotide (A) derived from the second polynucleotide ligation molecule. In the second cleavage step the second/synthesis strand is cleaved between the positions occupied by the nucleotides which are the first and second nucleotides immediately next to the universal nucleotide in the second/synthesis strand in the direction distal to the helper strand. Cleavage leaves in place a blunt-ended cleaved end of the scaffold polynucleotide with the terminal ligatable nucleotide of the second strand paired with the terminal ligatable nucleotide of the first strand.

FIG. 5. Scheme of Exemplary Method Version 5 of the Invention.

Scheme showing a first synthesis cycle according to exemplary method version 5 of the invention.

The method comprises cycles of provision of a scaffold polynucleotide, ligation of a first polynucleotide ligation molecule to the scaffold polynucleotide, a first cleavage step leading to the incorporation of one nucleotide into the first strand of the scaffold polynucleotide, ligation of a second polynucleotide ligation molecule to the scaffold polynucleotide, and a second cleavage step leading to the incorporation of one nucleotide into the second strand of the scaffold polynucleotide.

The scheme shows the provision of a scaffold polynucleotide (501, 506). One end of the scaffold polynucleotide is to be extended by the incorporation of a nucleotide of the predefined sequence into both strands (shown as the upper end of the scaffold polynucleotide). The opposite end of the scaffold polynucleotide (shown as the lower end, labelled 3′ and 5′) is shown not to be extended. The scaffold polynucleotide comprises a first strand (dotted lines) and a second strand (dotted-and-dashed lines) hybridised thereto. The end of the scaffold polynucleotide to be extended is shown with a blunt end. The terminal nucleotide of the first strand is depicted as “X” and is paired with the terminal nucleotide of the second strand, also depicted as “X”. The terminal nucleotides of the first and second strands at the end of the scaffold polynucleotide to be extended are ligatable nucleotides. These two nucleotides can be any two nucleotides or analogs or derivatives thereof, and are not limited to being a naturally complementary pair of nucleotides.

The scheme shows the provision of a first polynucleotide ligation molecule (502, 507; structure in the top right of the Figure). The polynucleotide ligation molecule comprises a helper strand (dashed line), a synthesis strand (solid line) hybridised thereto and a complementary ligation end. The terminal nucleotide of the synthesis strand of the complementary ligation end is a ligatable first nucleotide of the predefined sequence to be incorporated into the first strand, is depicted as “T” (thymine) and is paired with the terminal nucleotide of the helper strand of the complementary ligation end in a bunt end. The terminal nucleotide of the helper strand is depicted as a non-ligatable “A” (adenine). The complementary ligation end comprises a universal nucleotide (depicted as “Un”) in the synthesis strand and which is paired with a partner nucleotide in the helper strand (depicted as “X”) and which is the penultimate nucleotide in the helper strand. T, A and X are depicted purely for illustration and can be any nucleotides or analogs or derivatives thereof. It is not necessary for paired nucleotides to comprise naturally complementary nucleotides.

The scheme shows the ligation of the synthesis strand of the first polynucleotide ligation molecule (502, 507) to the first strand of the scaffold polynucleotide and the creation of single-stranded break (“nick”) between the helper strand of the first polynucleotide ligation molecule and the second strand.

The scheme shows a first cleavage step (503, 508) comprising cleaving the first/synthesis strand (jagged arrowhead) at a cleavage site defined by a sequence comprising the universal nucleotide. Cleavage releases the first polynucleotide ligation molecule comprising the universal nucleotide and leads to the retention in the scaffold polynucleotide of the thymine nucleotide derived from the first polynucleotide ligation molecule. In the first cleavage step the first/synthesis strand is cleaved between the positions occupied by the universal nucleotide and the nucleotide which occupies the next nucleotide position in the first/synthesis strand in the direction distal to the helper strand. Cleavage leaves in place a single-base overhang at the cleaved end of the scaffold polynucleotide with the terminal ligatable nucleotide of the first strand overhanging the terminal ligatable nucleotide of the second strand.

The scheme shows the provision of a second polynucleotide ligation molecule (504, 509). The polynucleotide ligation molecule comprises a helper strand (dashed line), a synthesis strand (solid line) hybridised thereto and a complementary ligation end. The terminal nucleotide of the synthesis strand of the complementary ligation end is a first nucleotide of the predefined sequence to be incorporated into the second strand, is depicted as a ligatable “A” (adenine) and overhangs the terminal nucleotide of the helper strand of the complementary ligation end in a single-nucleotide overhang. The terminal nucleotide of the helper strand is depicted as a non-ligatable nucleotide “X” and is paired with the universal nucleotide (depicted as “Un”), which is the penultimate nucleotide of the synthesis strand at the complementary ligation end. A and X are depicted purely for illustration and can be any nucleotides or analogs or derivatives thereof. It is not necessary for paired nucleotides to comprise naturally complementary nucleotides.

The scheme shows the ligation of the synthesis strand of the second polynucleotide ligation molecule (504, 509) to the second strand of the scaffold polynucleotide and the creation of single-stranded break (“nick”) between the helper strand of the second polynucleotide ligation molecule and the first strand.

The scheme shows a second cleavage step (505, 510) comprising cleaving the second/synthesis strand (jagged arrowhead) at a cleavage site defined by a sequence comprising the universal nucleotide. Cleavage releases the second polynucleotide ligation molecule comprising the universal nucleotide and leads to the retention in the scaffold polynucleotide of the adenine nucleotide (A) derived from the second polynucleotide ligation molecule. In the second cleavage step the second/synthesis strand is cleaved between the position occupied by the universal nucleotide and the nucleotide which occupies the next nucleotide position in the second/synthesis strand in the direction distal to the helper strand. Cleavage leaves in place a blunt-ended cleaved end of the scaffold polynucleotide with the terminal ligatable nucleotide of the second strand paired with the terminal ligatable nucleotide of the first strand.

FIG. 6. Scheme of Exemplary Method Version 6 of the Invention.

Scheme showing a first synthesis cycle according to exemplary method version 6 of the invention.

The method comprises cycles of provision of a scaffold polynucleotide, ligation of a first polynucleotide ligation molecule to the scaffold polynucleotide, a first cleavage step leading to the incorporation of one nucleotide into the first strand of the scaffold polynucleotide, ligation of a second polynucleotide ligation molecule to the scaffold polynucleotide, and a second cleavage step leading to the incorporation of one nucleotide into the second strand of the scaffold polynucleotide.

The scheme shows the provision of a scaffold polynucleotide (601, 606). One end of the scaffold polynucleotide is to be extended by the incorporation of a nucleotide of the predefined sequence into both strands (shown as the upper end of the scaffold polynucleotide). The opposite end of the scaffold polynucleotide (shown as the lower end, labelled 3′ and 5′) is shown not to be extended. The scaffold polynucleotide comprises a first strand (dotted lines) and a second strand (dotted-and-dashed lines) hybridised thereto.

The end of the scaffold polynucleotide to be extended is shown with a blunt end. The terminal nucleotide of the first strand is depicted as “X” and is paired with the terminal nucleotide of the second strand, also depicted as “X”. The terminal nucleotides of the first and second strands at the end of the scaffold polynucleotide to be extended are ligatable nucleotides. These two nucleotides can be any two nucleotides or analogs or derivatives thereof, and are not limited to being a naturally complementary pair of nucleotides.

The scheme shows the provision of a first polynucleotide ligation molecule (602, 607; structure in the top right of the Figure). The polynucleotide ligation molecule comprises a helper strand (dashed line), a synthesis strand (solid line) hybridised thereto and a complementary ligation end. The terminal nucleotide of the synthesis strand of the complementary ligation end is a ligatable first nucleotide of the predefined sequence to be incorporated into the first strand, is depicted as “T” (thymine) and is paired with the terminal nucleotide of the helper strand of the complementary ligation end in a bunt end. The terminal nucleotide of the helper strand is depicted as a non-ligatable “A” (adenine). The penultimate nucleotides of both the synthesis strand and the helper strand are paired and are depicted as “X”. The complementary ligation end comprises a universal nucleotide (depicted as “Un”) in the synthesis strand and which is paired with a partner nucleotide in the helper strand (depicted as “X”) and which occupies the position immediately next to the penultimate nucleotide in the helper strand in the direction distal to the complementary ligation end. T, A and X are depicted purely for illustration, and can be any nucleotides or analogs or derivatives thereof. It is not necessary for paired nucleotides to comprise naturally complementary nucleotides.

The scheme shows the ligation of the synthesis strand of the first polynucleotide ligation molecule (602, 607) to the first strand of the scaffold polynucleotide and the creation of single-stranded break (“nick”) between the helper strand of the first polynucleotide ligation molecule and the second strand.

The scheme shows a first cleavage step (603, 608) comprising cleaving the first/synthesis strand (jagged arrowhead) at a cleavage site defined by a sequence comprising the universal nucleotide. Cleavage releases the first polynucleotide ligation molecule comprising the universal nucleotide and leads to the retention in the scaffold polynucleotide of the thymine nucleotide derived from the first polynucleotide ligation molecule. In the first cleavage step the first/synthesis strand is cleaved between the positions occupied by the nucleotides which are the first and second nucleotides immediately next to the universal nucleotide in the first/synthesis strand in the direction distal to the helper strand. Cleavage leaves in place a single-base overhang at the cleaved end of the scaffold polynucleotide with the terminal ligatable nucleotide of the first strand overhanging the terminal ligatable nucleotide of the second strand.

The scheme shows the provision of a second polynucleotide ligation molecule (604, 609). The polynucleotide ligation molecule comprises a helper strand (dashed line), a synthesis strand (solid line) hybridised thereto and a complementary ligation end. The terminal nucleotide of the synthesis strand of the complementary ligation end is a first nucleotide of the predefined sequence to be incorporated into the second strand, is depicted as a ligatable “A” (adenine) and overhangs the terminal nucleotide of the helper strand of the complementary ligation end in a single-nucleotide overhang. The terminal nucleotide of the helper strand is depicted as a non-ligatable nucleotide “X” and is paired with the universal nucleotide (depicted as “Un”), which is the penultimate nucleotide of the synthesis strand at the complementary ligation end. A and X are depicted purely for illustration and can be any nucleotides or analogs or derivatives thereof. It is not necessary for paired nucleotides to comprise naturally complementary nucleotides.

The scheme shows the ligation of the synthesis strand of the second polynucleotide ligation molecule (604, 609) to the second strand of the scaffold polynucleotide and the creation of single-stranded break (“nick”) between the helper strand of the second polynucleotide ligation molecule and the first strand.

The scheme shows a second cleavage step (605, 610) comprising cleaving the second/synthesis strand (jagged arrowhead) at a cleavage site defined by a sequence comprising the universal nucleotide. Cleavage releases the second polynucleotide ligation molecule comprising the universal nucleotide and leads to the retention in the scaffold polynucleotide of the adenine nucleotide (A) derived from the second polynucleotide ligation molecule. In the second cleavage step the second/synthesis strand is cleaved between the position occupied by the universal nucleotide and the nucleotide which occupies the next nucleotide position in the second/synthesis strand in the direction distal to the helper strand. Cleavage leaves in place a blunt-ended cleaved end of the scaffold polynucleotide with the terminal ligatable nucleotide of the second strand paired with the terminal ligatable nucleotide of the first strand.

FIG. 7. Scheme of Exemplary Method Version 7 of the Invention.

Scheme showing a first synthesis cycle according to exemplary method version 7 of the invention, which is a variant of exemplary method version 3 of the invention.

The method comprises cycles of provision of a scaffold polynucleotide, ligation of a first polynucleotide ligation molecule to the scaffold polynucleotide, a first cleavage step leading to the incorporation of one nucleotide into the first strand of the scaffold polynucleotide, ligation of a second polynucleotide ligation molecule to the scaffold polynucleotide, and a second cleavage step leading to the incorporation of one nucleotide into the second strand of the scaffold polynucleotide.

The scheme shows the provision of a scaffold polynucleotide (701, 706). One end of the scaffold polynucleotide is to be extended by the incorporation of a nucleotide of the predefined sequence into both strands (shown as the upper end of the scaffold polynucleotide). The opposite end of the scaffold polynucleotide (shown as the lower end, labelled 3′ and 5′) is shown not to be extended. The scaffold polynucleotide comprises a first strand (dotted lines) and a second strand (dotted-and-dashed lines) hybridised thereto. The end of the scaffold polynucleotide to be extended is shown with a blunt end. The terminal nucleotide of the first strand is depicted as “X” and is paired with the terminal nucleotide of the second strand, also depicted as “X”. The terminal nucleotides of the first and second strands at the end of the scaffold polynucleotide to be extended are ligatable nucleotides. These two nucleotides can be any two nucleotides or analogs or derivatives thereof, and are not limited to being a naturally complementary pair of nucleotides.

The scheme shows the provision of a first polynucleotide ligation molecule (702, 707; structure in the top right of the Figure). The polynucleotide ligation molecule comprises a helper strand (dashed line), a synthesis strand (solid line) hybridised thereto and a complementary ligation end. The terminal nucleotide of the synthesis strand of the complementary ligation end is a ligatable first nucleotide of the predefined sequence to be incorporated into the first strand, is depicted as “T” (thymine) and is paired with the terminal nucleotide of the helper strand of the complementary ligation end in a bunt end. The terminal nucleotide of the helper strand is depicted as a non-ligatable “A” (adenine). The complementary ligation end comprises a universal nucleotide (depicted as “Un”) in the synthesis strand and which is paired with a partner nucleotide in the helper strand (depicted as “X”). The penultimate nucleotide of the synthesis strand is depicted as “G” (guanine) and penultimate nucleotide of the helper strand is depicted as “C” (cytosine). T, A, G, C and X are depicted purely for illustration and can be any nucleotides or analogs or derivatives thereof. It is not necessary for paired nucleotides to comprise naturally complementary nucleotides. The universal nucleotide occupies the nucleotide position immediately next to the penultimate nucleotide in the synthesis strand in the direction distal to the complementary ligation end.

The scheme shows the ligation of the synthesis strand of the first polynucleotide ligation molecule (702, 707) to the first strand of the scaffold polynucleotide and the creation of single-stranded break (“nick”) between the helper strand of the first polynucleotide ligation molecule and the second strand.

The scheme shows a first cleavage step (703, 708) comprising cleaving the first/synthesis strand (jagged arrowhead) at a cleavage site defined by a sequence comprising the universal nucleotide. Cleavage releases the first polynucleotide ligation molecule comprising the universal nucleotide and leads to the retention in the scaffold polynucleotide of the thymine and guanine nucleotides derived from the first polynucleotide ligation molecule. In the first cleavage step the first/synthesis strand is cleaved between the positions occupied by the universal nucleotide and the nucleotide which occupies the next nucleotide position in the first/synthesis strand in the direction distal to the helper strand. Cleavage leaves in place a two-base overhang at the cleaved end of the scaffold polynucleotide with the terminal and penultimate nucleotides of the first strand overhanging the terminal ligatable nucleotide of the second strand.

The scheme shows the provision of a second polynucleotide ligation molecule (704, 709). The polynucleotide ligation molecule comprises a helper strand (dashed line), a synthesis strand (solid line) hybridised thereto and a complementary ligation end. The terminal nucleotide of the synthesis strand of the complementary ligation end is a first nucleotide of the predefined sequence to be incorporated into the second strand, is depicted as a ligatable “A” (adenine). The penultimate nucleotide of the synthesis strand of the complementary ligation end is a second nucleotide of the predefined sequence to be incorporated into the second strand, is depicted as “C” (cytosine). The terminal and penultimate nucleotides of the synthesis strand overhang the terminal nucleotide of the helper strand of the complementary ligation end in a two-nucleotide overhang. The terminal nucleotide of the helper strand is depicted as a non-ligatable nucleotide “X” and is paired with the universal nucleotide (depicted as “Un”), which occupies the nucleotide position immediately next to the penultimate nucleotide in the synthesis strand in the direction distal to the complementary ligation end. A, C and X are depicted purely for illustration and can be any nucleotides or analogs or derivatives thereof. It is not necessary for paired nucleotides to comprise naturally complementary nucleotides.

The scheme shows the ligation of the synthesis strand of the second polynucleotide ligation molecule (704, 709) to the second strand of the scaffold polynucleotide and the creation of single-stranded break (“nick”) between the helper strand of the second polynucleotide ligation molecule and the first strand.

The scheme shows a second cleavage step (705, 710) comprising cleaving the second/synthesis strand (jagged arrowhead) at a cleavage site defined by a sequence comprising the universal nucleotide. Cleavage releases the second polynucleotide ligation molecule comprising the universal nucleotide and leads to the retention in the scaffold polynucleotide of the adenine (A) and cytosine (C) nucleotides derived from the second polynucleotide ligation molecule. In the second cleavage step the second/synthesis strand is cleaved between the position occupied by the universal nucleotide and the nucleotide which occupies the next nucleotide position in the second/synthesis strand in the direction distal to the helper strand. Cleavage leaves in place a blunt-ended cleaved end of the scaffold polynucleotide with the terminal ligatable nucleotide of the second strand paired with the terminal ligatable nucleotide of the first strand and with both first and second nucleotides incorporated into the first and second strands.

FIG. 8. Scheme of Exemplary Method Version 8 of the Invention.

Scheme showing a first synthesis cycle according to exemplary method version 8 of the invention, which is a variant of exemplary method version 3 of the invention.

The method comprises cycles of provision of a scaffold polynucleotide, ligation of a first polynucleotide ligation molecule to the scaffold polynucleotide, a first cleavage step leading to the incorporation of one nucleotide into the first strand of the scaffold polynucleotide, ligation of a second polynucleotide ligation molecule to the scaffold polynucleotide, and a second cleavage step leading to the incorporation of one nucleotide into the second strand of the scaffold polynucleotide.

The scheme shows the provision of a scaffold polynucleotide (801, 806). One end of the scaffold polynucleotide is to be extended by the incorporation of a nucleotide of the predefined sequence into both strands (shown as the upper end of the scaffold polynucleotide). The opposite end of the scaffold polynucleotide (shown as the lower end, labelled 3′ and 5′) is shown not to be extended. The scaffold polynucleotide comprises a first strand (dotted lines) and a second strand (dotted-and-dashed lines) hybridised thereto. The end of the scaffold polynucleotide to be extended is shown with a blunt end. The terminal nucleotide of the first strand is depicted as “X” and is paired with the terminal nucleotide of the second strand, also depicted as “X”. The terminal nucleotides of the first and second strands at the end of the scaffold polynucleotide to be extended are ligatable nucleotides. These two nucleotides can be any two nucleotides or analogs or derivatives thereof, and are not limited to being a naturally complementary pair of nucleotides.

The scheme shows the provision of a first polynucleotide ligation molecule (802, 807; structure in the top right of the Figure). The polynucleotide ligation molecule comprises a helper strand (dashed line), a synthesis strand (solid line) hybridised thereto and a complementary ligation end. The terminal nucleotide of the synthesis strand of the complementary ligation end is a ligatable first nucleotide of the predefined sequence to be incorporated into the first strand, is depicted as “T” (thymine) and is paired with the terminal nucleotide of the helper strand of the complementary ligation end in a bunt end. The terminal nucleotide of the helper strand is depicted as a non-ligatable “A” (adenine). The complementary ligation end comprises a universal nucleotide (depicted as “Un”) in the synthesis strand and which is paired with a partner nucleotide in the helper strand (depicted as “X”). The penultimate nucleotide of the synthesis strand is depicted as “G” (guanine) and penultimate nucleotide of the helper strand is depicted as “C” (cytosine). T, A, G, C and X are depicted purely for illustration and can be any nucleotides or analogs or derivatives thereof. It is not necessary for paired nucleotides to comprise naturally complementary nucleotides. The universal nucleotide occupies the nucleotide position immediately next to the penultimate nucleotide in the synthesis strand in the direction distal to the complementary ligation end.

The scheme shows the ligation of the synthesis strand of the first polynucleotide ligation molecule (802, 807) to the first strand of the scaffold polynucleotide and the creation of single-stranded break (“nick”) between the helper strand of the first polynucleotide ligation molecule and the second strand.

The scheme shows a first cleavage step (803, 808) comprising cleaving the first/synthesis strand (jagged arrowhead) at a cleavage site defined by a sequence comprising the universal nucleotide. Cleavage releases the first polynucleotide ligation molecule comprising the universal nucleotide and leads to the retention in the scaffold polynucleotide of the thymine and guanine nucleotides derived from the first polynucleotide ligation molecule. In the first cleavage step the first/synthesis strand is cleaved between the positions occupied by the universal nucleotide and the nucleotide which occupies the next nucleotide position in the first/synthesis strand in the direction distal to the helper strand. Cleavage leaves in place a two-base overhang at the cleaved end of the scaffold polynucleotide with the terminal and penultimate nucleotides of the first strand overhanging the terminal ligatable nucleotide of the second strand.

The scheme shows the provision of a second polynucleotide ligation molecule (804, 809). The polynucleotide ligation molecule comprises a helper strand (dashed line), a synthesis strand (solid line) hybridised thereto and a complementary ligation end. The terminal nucleotide of the synthesis strand of the complementary ligation end is a first nucleotide of the predefined sequence to be incorporated into the second strand, is depicted as a ligatable “A” (adenine). The penultimate nucleotide of the synthesis strand of the complementary ligation end is a second nucleotide of the predefined sequence to be incorporated into the second strand, is depicted as “C” (cytosine). The terminal and penultimate nucleotides of the synthesis strand overhang the terminal nucleotide of the helper strand of the complementary ligation end in a two-nucleotide overhang. The terminal nucleotide of the helper strand is depicted as a non-ligatable nucleotide “X” and is paired with the nucleotide which occupies the nucleotide position immediately next to the penultimate nucleotide in the synthesis strand in the direction distal to the complementary ligation end. The universal nucleotide occupies the fourth nucleotide position in the synthesis strand in the direction distal to the complementary ligation end. A, C and X are depicted purely for illustration and can be any nucleotides or analogs or derivatives thereof. It is not necessary for paired nucleotides to comprise naturally complementary nucleotides.

The scheme shows the ligation of the synthesis strand of the second polynucleotide ligation molecule (804, 809) to the second strand of the scaffold polynucleotide and the creation of single-stranded break (“nick”) between the helper strand of the second polynucleotide ligation molecule and the first strand.

The scheme shows a second cleavage step (805, 810) comprising cleaving the second/synthesis strand (jagged arrowhead) at a cleavage site defined by a sequence comprising the universal nucleotide. Cleavage releases the second polynucleotide ligation molecule comprising the universal nucleotide and leads to the retention in the scaffold polynucleotide of the adenine (A) and cytosine (C) nucleotides derived from the second polynucleotide ligation molecule. In the second cleavage step the second/synthesis strand is cleaved between the nucleotides which occupy the first and second positions next to the universal nucleotide in the second/synthesis strand in the direction distal to the helper strand. Cleavage leaves in place a blunt-ended cleaved end of the scaffold polynucleotide with the terminal ligatable nucleotide of the second strand paired with the terminal ligatable nucleotide of the first strand and with both first and second nucleotides incorporated into the first and second strands.

FIG. 9. Scheme of Exemplary Method Version 9 of the Invention.

Scheme showing a first synthesis cycle according to exemplary method version 9 of the invention, which is a variant of exemplary method version 5 of the invention.

The method comprises cycles of provision of a scaffold polynucleotide, ligation of a first polynucleotide ligation molecule to the scaffold polynucleotide, a first cleavage step leading to the incorporation of one nucleotide into the first strand of the scaffold polynucleotide, ligation of a second polynucleotide ligation molecule to the scaffold polynucleotide, and a second cleavage step leading to the incorporation of one nucleotide into the second strand of the scaffold polynucleotide.

The scheme shows the provision of a scaffold polynucleotide (901, 906). One end of the scaffold polynucleotide is to be extended by the incorporation of a nucleotide of the predefined sequence into both strands (shown as the upper end of the scaffold polynucleotide). The opposite end of the scaffold polynucleotide (shown as the lower end, labelled 3′ and 5′) is shown not to be extended. The scaffold polynucleotide comprises a first strand (dotted lines) and a second strand (dotted-and-dashed lines) hybridised thereto.

The end of the scaffold polynucleotide to be extended is shown with a blunt end. The terminal nucleotide of the first strand is depicted as “X” and is paired with the terminal nucleotide of the second strand, also depicted as “X”. The terminal nucleotides of the first and second strands at the end of the scaffold polynucleotide to be extended are ligatable nucleotides. These two nucleotides can be any two nucleotides or analogs or derivatives thereof, and are not limited to being a naturally complementary pair of nucleotides.

The scheme shows the provision of a first polynucleotide ligation molecule (902, 907; structure in the top right of the Figure). The polynucleotide ligation molecule comprises a helper strand (dashed line), a synthesis strand (solid line) hybridised thereto and a complementary ligation end. The terminal nucleotide of the synthesis strand of the complementary ligation end is a ligatable first nucleotide of the predefined sequence to be incorporated into the first strand, is depicted as “T” (thymine) and is paired with the terminal nucleotide of the helper strand of the complementary ligation end in a bunt end. The terminal nucleotide of the helper strand is depicted as a non-ligatable “A” (adenine). The complementary ligation end comprises a universal nucleotide (depicted as “Un”) in the synthesis strand and which is paired with a partner nucleotide in the helper strand (depicted as “X”). The penultimate nucleotide of the synthesis strand is depicted as “C” (cytosine) and the penultimate nucleotide of the helper strand is depicted as “G” (guanine). T, A, G, C and X are depicted purely for illustration and can be any nucleotides or analogs or derivatives thereof. It is not necessary for paired nucleotides to comprise naturally complementary nucleotides. The universal nucleotide occupies the nucleotide position immediately next to the penultimate nucleotide in the synthesis strand in the direction distal to the complementary ligation end.

The scheme shows the ligation of the synthesis strand of the first polynucleotide ligation molecule (902, 907) to the first strand of the scaffold polynucleotide and the creation of single-stranded break (“nick”) between the helper strand of the first polynucleotide ligation molecule and the second strand.

The scheme shows a first cleavage step (903, 908) comprising cleaving the first/synthesis strand (jagged arrowhead) at a cleavage site defined by a sequence comprising the universal nucleotide. Cleavage releases the first polynucleotide ligation molecule comprising the universal nucleotide and leads to the retention in the scaffold polynucleotide of the thymine and guanine nucleotides derived from the first polynucleotide ligation molecule. In the first cleavage step the first/synthesis strand is cleaved between the positions occupied by the universal nucleotide and the nucleotide which occupies the next nucleotide position in the first/synthesis strand in the direction distal to the helper strand. Cleavage leaves in place a two-base overhang at the cleaved end of the scaffold polynucleotide with the terminal and penultimate nucleotides of the first strand overhanging the terminal ligatable nucleotide of the second strand.

The scheme shows the provision of a second polynucleotide ligation molecule (904, 909). The polynucleotide ligation molecule comprises a helper strand (dashed line), a synthesis strand (solid line) hybridised thereto and a complementary ligation end. The terminal nucleotide of the synthesis strand of the complementary ligation end is a first nucleotide of the predefined sequence to be incorporated into the second strand, is depicted as a ligatable “A” (adenine). The penultimate nucleotide of the synthesis strand of the complementary ligation end is a second nucleotide of the predefined sequence to be incorporated into the second strand, is depicted as “G” (guanine). The terminal and penultimate nucleotides of the synthesis strand overhang the terminal nucleotide of the helper strand of the complementary ligation end in a two-nucleotide overhang. The terminal nucleotide of the helper strand is depicted as a non-ligatable nucleotide “X” and is paired with the universal nucleotide (depicted as “Un”), which occupies the nucleotide position immediately next to the penultimate nucleotide in the synthesis strand in the direction distal to the complementary ligation end. A, G and X are depicted purely for illustration and can be any nucleotides or analogs or derivatives thereof. It is not necessary for paired nucleotides to comprise naturally complementary nucleotides.

The scheme shows the ligation of the synthesis strand of the second polynucleotide ligation molecule (904, 909) to the second strand of the scaffold polynucleotide and the creation of single-stranded break (“nick”) between the helper strand of the second polynucleotide ligation molecule and the first strand.

The scheme shows a second cleavage step (905, 910) comprising cleaving the second/synthesis strand (jagged arrowhead) at a cleavage site defined by a sequence comprising the universal nucleotide. Cleavage releases the second polynucleotide ligation molecule comprising the universal nucleotide and leads to the retention in the scaffold polynucleotide of the adenine (A) and guanine (G) nucleotides derived from the second polynucleotide ligation molecule. In the second cleavage step the second/synthesis strand is cleaved between the position occupied by the universal nucleotide and the nucleotide which occupies the next nucleotide position in the second/synthesis strand in the direction distal to the helper strand. Cleavage leaves in place a blunt-ended cleaved end of the scaffold polynucleotide with the terminal ligatable nucleotide of the second strand paired with the terminal ligatable nucleotide of the first strand and with both first and second nucleotides incorporated into the first and second strands.

FIG. 10. Scheme of Exemplary Method Version 10 of the Invention.

Scheme showing a first synthesis cycle according to exemplary method version 10 of the invention, which is a variant of exemplary method version 5 of the invention.

The method comprises cycles of provision of a scaffold polynucleotide, ligation of a first polynucleotide ligation molecule to the scaffold polynucleotide, a first cleavage step leading to the incorporation of one nucleotide into the first strand of the scaffold polynucleotide, ligation of a second polynucleotide ligation molecule to the scaffold polynucleotide, and a second cleavage step leading to the incorporation of one nucleotide into the second strand of the scaffold polynucleotide.

The scheme shows the provision of a scaffold polynucleotide (1001, 1006). One end of the scaffold polynucleotide is to be extended by the incorporation of a nucleotide of the predefined sequence into both strands (shown as the upper end of the scaffold polynucleotide). The opposite end of the scaffold polynucleotide (shown as the lower end, labelled 3′ and 5′) is shown not to be extended. The scaffold polynucleotide comprises a first strand (dotted lines) and a second strand (dotted-and-dashed lines) hybridised thereto. The end of the scaffold polynucleotide to be extended is shown with a blunt end. The terminal nucleotide of the first strand is depicted as “X” and is paired with the terminal nucleotide of the second strand, also depicted as “X”. The terminal nucleotides of the first and second strands at the end of the scaffold polynucleotide to be extended are ligatable nucleotides. These two nucleotides can be any two nucleotides or analogs or derivatives thereof, and are not limited to being a naturally complementary pair of nucleotides.

The scheme shows the provision of a first polynucleotide ligation molecule (1002, 1007; structure in the top right of the Figure). The polynucleotide ligation molecule comprises a helper strand (dashed line), a synthesis strand (solid line) hybridised thereto and a complementary ligation end. The terminal nucleotide of the synthesis strand of the complementary ligation end is a ligatable first nucleotide of the predefined sequence to be incorporated into the first strand, is depicted as “T” (thymine) and is paired with the terminal nucleotide of the helper strand of the complementary ligation end in a bunt end. The terminal nucleotide of the helper strand is depicted as a non-ligatable “A” (adenine). The penultimate nucleotide of the synthesis strand of the complementary ligation end is a second nucleotide of the predefined sequence to be incorporated into the first strand, is depicted as “C” (cytosine). The complementary ligation end comprises a universal nucleotide (depicted as “Un”) in the synthesis strand and which is paired with a partner nucleotide in the helper strand (depicted as “X”). The universal nucleotide occupies the fourth nucleotide position in the synthesis strand in the direction distal to the complementary ligation end. T, A, G, C and X are depicted purely for illustration and can be any nucleotides or analogs or derivatives thereof. It is not necessary for paired nucleotides to comprise naturally complementary nucleotides.

The scheme shows the ligation of the synthesis strand of the first polynucleotide ligation molecule (1002, 1007) to the first strand of the scaffold polynucleotide and the creation of single-stranded break (“nick”) between the helper strand of the first polynucleotide ligation molecule and the second strand.

The scheme shows a first cleavage step (1003, 1008) comprising cleaving the first/synthesis strand (jagged arrowhead) at a cleavage site defined by a sequence comprising the universal nucleotide. Cleavage releases the first polynucleotide ligation molecule comprising the universal nucleotide and leads to the retention in the scaffold polynucleotide of the thymine and cytosine nucleotides derived from the first polynucleotide ligation molecule. In the first cleavage step the first/synthesis strand is cleaved between the nucleotides which occupy the first and second positions next to the universal nucleotide in the first/synthesis strand in the direction distal to the helper strand. Cleavage leaves in place a two-base overhang at the cleaved end of the scaffold polynucleotide with the terminal and penultimate nucleotides of the first strand overhanging the terminal ligatable nucleotide of the second strand.

The scheme shows the provision of a second polynucleotide ligation molecule (1004, 1009). The polynucleotide ligation molecule comprises a helper strand (dashed line), a synthesis strand (solid line) hybridised thereto and a complementary ligation end. The terminal nucleotide of the synthesis strand of the complementary ligation end is a first nucleotide of the predefined sequence to be incorporated into the second strand, is depicted as a ligatable “A” (adenine). The penultimate nucleotide of the synthesis strand of the complementary ligation end is a second nucleotide of the predefined sequence to be incorporated into the second strand, is depicted as “G” (guanine). The terminal and penultimate nucleotides of the synthesis strand overhang the terminal nucleotide of the helper strand of the complementary ligation end in a two-nucleotide overhang. The terminal nucleotide of the helper strand is depicted as a non-ligatable nucleotide “X” and is paired with the universal nucleotide (depicted as “Un”), which occupies the nucleotide position immediately next to the penultimate nucleotide in the synthesis strand in the direction distal to the complementary ligation end. A, G and X are depicted purely for illustration and can be any nucleotides or analogs or derivatives thereof. It is not necessary for paired nucleotides to comprise naturally complementary nucleotides.

The scheme shows the ligation of the synthesis strand of the second polynucleotide ligation molecule (1004, 1009) to the second strand of the scaffold polynucleotide and the creation of single-stranded break (“nick”) between the helper strand of the second polynucleotide ligation molecule and the first strand.

The scheme shows a second cleavage step (1005, 1010) comprising cleaving the second/synthesis strand (jagged arrowhead) at a cleavage site defined by a sequence comprising the universal nucleotide. Cleavage releases the second polynucleotide ligation molecule comprising the universal nucleotide and leads to the retention in the scaffold polynucleotide of the adenine (A) and guanine (G) nucleotides derived from the second polynucleotide ligation molecule. In the second cleavage step the second/synthesis strand is cleaved between the position occupied by the universal nucleotide and the nucleotide which occupies the next nucleotide position in the second/synthesis strand in the direction distal to the helper strand. Cleavage leaves in place a blunt-ended cleaved end of the scaffold polynucleotide with the terminal ligatable nucleotide of the second strand paired with the terminal ligatable nucleotide of the first strand and with both first and second nucleotides incorporated into the first and second strands.

FIG. 11. Scheme of Exemplary Method Version 1.

Scheme showing a first synthesis cycle according to exemplary method version 1 of the Examples section. This method is provided for illustrative support only and is not within the scope of the claimed invention. The method comprises a cycle of provision of a scaffold polynucleotide, incorporation, cleavage, ligation and deprotection. The scheme shows the incorporation of a thymine nucleotide in the first synthesis cycle (101, 102) and its pairing opposite a partner adenine nucleotide (104), as well as the provision of a scaffold polynucleotide (106) for use in the next synthesis cycle. This pair is shown for illustration purposes only and is not limiting, it can be any pair depending on the required predefined sequence. Nucleotide Z can be any nucleotide. Nucleotide X can be any appropriate nucleotide. The Figure also shows reference signs corresponding to a second synthesis cycle.

FIG. 12. Scheme of Exemplary Method Version 2.

Scheme showing a first synthesis cycle according to exemplary method version 2 of the Examples section. This method is provided for illustrative support only and is not within the scope of the claimed invention. The method comprises a cycle of provision of a scaffold polynucleotide, incorporation, cleavage, ligation and deprotection. The scheme shows the incorporation in the first cycle (201, 202) of a thymine nucleotide and its pairing opposite a partner adenine nucleotide (204), as well as the provision of a scaffold polynucleotide (206) comprising a guanine for pairing with a cytosine in the next synthesis cycle. These pairs are shown for illustration purposes only and are not limiting, they can be any pairs depending on the required predefined sequence. Nucleotide Z can be any nucleotide. Nucleotide X can be any appropriate nucleotide. The Figure also shows reference signs corresponding to a second synthesis cycle.

FIG. 13. Scheme of Exemplary Method Version 3.

Scheme showing a first synthesis cycle according to exemplary method version 3 of the Examples section. This method is provided for illustrative support only and is not within the scope of the claimed invention. The method comprises a cycle of provision of a scaffold polynucleotide, incorporation, cleavage, ligation and deprotection. The scheme shows the incorporation in the first cycle (301, 302) of a thymine nucleotide and its pairing opposite a partner adenine nucleotide (304), as well as the provision of a scaffold polynucleotide (306) for use in the next synthesis cycle. This pair is shown for illustration purposes only and is not limiting, it can be any pair depending on the required predefined sequence. The scheme also shows a cytosine-guanine pair as a component of the scaffold polynucleotide and which is not part of the predefined sequence. This pair is also shown for illustration purposes only and is not limiting, it can be any pair. Nucleotide Z can be any nucleotide. Nucleotide X can be any appropriate nucleotide.

FIG. 14. Scheme of Exemplary Method Version 4.

Scheme showing a first synthesis cycle according to exemplary method version 4 of the Examples section. This method is provided for illustrative support only and is not within the scope of the claimed invention. The method comprises a cycle of provision of a scaffold polynucleotide, incorporation, cleavage, ligation and deprotection. The scheme shows the incorporation in the first cycle (401, 402) of a thymine nucleotide and its pairing opposite a partner universal nucleotide (404), as well as the provision of a scaffold polynucleotide (406) comprising a guanine for pairing with a cytosine in the next synthesis cycle. These pairs are shown for illustration purposes only and are not limiting, they can be any pairs depending on the required predefined sequence. Nucleotides X, Y and Z can be any nucleotide.

FIG. 15. Scheme of Exemplary Method Version 5.

Scheme showing a first synthesis cycle according to exemplary method version 5 of the Examples section. This method is provided for illustrative support only and is not within the scope of the claimed invention. The method comprises a cycle of provision of a scaffold polynucleotide, incorporation, cleavage, ligation and deprotection. The scheme shows the incorporation in the first cycle (501, 502) of a thymine nucleotide and its pairing opposite a partner adenine nucleotide (504), as well as the provision of a scaffold polynucleotide (506) comprising a guanine for pairing with a cytosine in the next synthesis cycle. The scheme also shows a cytosine-guanine pair (position n−2) as a component of the scaffold polynucleotide and which is not part of the predefined sequence. These pairs are shown for illustration purposes only and are not limiting, they can be any pairs depending on the required predefined sequence. Nucleotides X, Y and Z can be any nucleotide.

FIG. 16. Scheme Showing Surface Immobilization of Scaffold Polynucleotides.

Schemes show (a to h) possible example hairpin loop configurations of scaffold polynucleotides and their immobilisation to surfaces.

Schemes (i and j) show examples of surface chemistries for attaching polynucleotides to surfaces. The examples show double-stranded embodiments wherein both strands are connected via a hairpin, but the same chemistries may be used for attaching one or both strands of an unconnected double-stranded polynucleotide.

FIG. 17. Absence of Helper Strand—Incorporation.

a) Scheme showing incorporation step highlighted in dashed box.

b) Evaluation of DNA polymerases for incorporation of 3′-O-modified-dTTPs opposite inosine. The Figure depicts a gel showing results of incorporation of 3′-O-modified-dTTPs by various DNA polymerases (Bst, Deep Vent (Exo-), Therminator I and Therminator IX) in presence of Mn²⁺ ions at 50° C. Lane 1: Incorporation of 3′-O-allyl-dTTPs using Bst DNA polymerase. Lane 2: Incorporation of 3′-O-azidomethyl-dTTPs using Bst DNA polymerase. Lane 3: Incorporation of 3′-O-allyl-dTTPs using Deep vent (exo-) DNA polymerase. Lane 4: Incorporation of 3′-O-azidomethyl-dTTPs using Deep vent (exo-) DNA polymerase. Lane 5: Incorporation of 3′-O-allyl-dTTPs using Therminator I DNA polymerase. Lane 6: Incorporation of 3′-O-azidomethyl-dTTPs using Therminator I DNA polymerase. Lane 7: Incorporation of 3′-O-allyl-dTTPs using Therminator IX DNA polymerase. Lane 8: Incorporation of 3′-O-azidomethyl-dTTPs using Therminator IX DNA polymerase.

c) Evaluation of DNA polymerases for incorporation of 3′-O-modified-dTTPs opposite inosine. Results of incorporation using various DNA polymerases.

d) Evaluation of the temperature on the incorporation using Therminator IX DNA polymerase. The Figure depicts a gel showing results of incorporation of 3′-modified-dTTP opposite inosine in presence of Mn²⁺ ions using Therminator IX DNA polymerase at various temperatures. Lane 1: Incorporation of 3′-O-allyl-dTTPs at 37° C. Lane 2: Incorporation of 3′-O-azidomethyl-dTTPs at 37° C. Lane 3: Incorporation of 3′-O-allyl-dTTPs at 50° C. Lane 4: Incorporation of 3′-O-azidomethyl-dTTPs at 50° C. Lane 5: Incorporation of 3′-O-allyl-dTTPs at 65° C. Lane 6: Incorporation of 3′-O-azidomethyl-dTTPs at 65° C.

e) Evaluation of the temperature on the incorporation using Therminator IX DNA polymerase. Results of incorporation performed at different temperatures.

f) Evaluation of the presence of Mn²⁺ on the incorporation using Therminator IX DNA polymerase. The Figure depicts a gel showing results of incorporation of 3′-O-modified-dTTP opposite inosine at 65° C. Lane S: Standards. Lane 1: Incorporation of 3′-O-allyl-dTTPs without Mn²⁺ ions. Lane 2: Incorporation of 3′-O-azidomethyl-dTTPs without Mn²⁺ ions. Lane 3: Incorporation of 3′-O-allyl-dTTPs in presence of Mn²⁺ ions. Lane 4: Incorporation of 3′-O-azidomethyl-dTTPs in presence of Mn²⁺ ions.

g) Evaluation of the presence of Mn²⁺ on the incorporation using Therminator IX DNA polymerase. Results of incorporation in presence and absence of Mn²⁺ ions.

h) Oligonucleotides used for study of the incorporation step.

FIG. 18. Absence of Helper Strand—Cleavage.

a) Scheme showing cleavage of hybridized polynucleotide strands in the absence of a helper strand. Cleavage step is highlighted in dashed box.

b) Gel showing cleavage of oligonucleotide with hAAG and 0.2M NaOH (strong base) at 37° C. and room temperature 24° C. respectively. Lane 1. Starting oligonucleotide. Lane 2 which was a positive control that contained both full length strands showed a higher yield of cleaved to uncleaved DNA ratio of 90%:10%. Lane 3 which included the cleavage reaction without a helper strand showed a low percentage yield of cleaved to uncleaved DNA ratio of 10%:90%.

c) Gel showing cleavage of oligonucleotide with hAAG and Endo VIII at 37° C. Lane 2 which was a positive control that contained both full length strands showed a higher yield of cleaved to uncleaved DNA ratio of ˜90%:10%. Lane 3 which included the cleavage reaction without a helper strand showed a low percentage yield of cleaved to uncleaved DNA ratio of ˜7%:93%.

d) A summary of cleavage of oligonucleotide with hAAG/Endo VIII and hAAG/Chemical base.

e) Oligonucleotides used for study of the cleavage step.

FIG. 19. Absence of Helper Strand—Ligation.

a) Scheme showing ligation of hybridized polynucleotide strands in the absence of a helper strand. Ligation step highlighted in dashed box.

b) Gel showing ligation of Oligonucleotides with Quick T4 DNA ligase at room temperature (24° C.) in the absence of a helper strand. Lane 1 contained a mixture of the 36mers TAMRA single stranded oligos and 18mers TAMRA single stranded oligos. These oligos served reference bands.

c) Oligonucleotides used for study of the ligation step.

FIG. 20. Version 1 Chemistry with Helper Strand—Incorporation.

a) Scheme showing incorporation step highlighted in dashed box.

b) Oligonucleotides applicable for study of the incorporation step.

FIG. 21. Version 1 Chemistry with Helper Strand—Cleavage.

a) Scheme showing cleavage of hybridized polynucleotide strands in the absence of a helper strand. Cleavage step is highlighted in dashed box.

b) Gel showing cleavage of Oligonucleotide with hAAG and 0.2M NaOH (strong base) at 37° C. and room temperature 24° C. respectively. Lane 1. Starting oligonucleotide. Lane 2 which was a positive control that contained both full length strands showed a higher yield of cleaved to uncleaved DNA ratio of 90%:10%. Lane 3 which included the cleavage reaction without a helper strand showed a low percentage yield of cleaved to uncleaved DNA ratio of 10%:90%. Lane 4 which included the cleavage reaction with a helper strand showed an equal percentage yield of cleaved to uncleaved DNA ratio of 50%:50%.

c) Evaluation of Endonuclease VIII for cleavage of abasic sites. Gel shows cleavage of oligonucleotide with hAAG and Endo VIII at 37° C. Lane 2 which was a positive control that contained both full length strands showed a higher yield of cleaved to uncleaved DNA ratio of ˜90%:10%. Lane 3 which included the cleavage reaction without a helper strand showed a low percentage yield of cleaved to uncleaved DNA ratio of ˜7%:93%. Lane 4 which included the cleavage reaction with a helper strand showed an low percentage yield of cleaved to uncleaved DNA ratio of 10%:90%.

d) Evaluation of N,N′-dimethylethylenediamine for cleavage of abasic sites. Gel shows cleavage of oligonucleotide with hAAG and 100 mM N,N′-dimethylethylenediamine at 37° C. Lane 1. Starting oligonucleotide. Lane 2 which was a positive control that contained both full length strands showed a 100% cleaved DNA. Lane 3 which included the cleavage reaction with a helper strand showed a higher percentage yield of cleaved to uncleaved DNA ratio of 90%:10%.

e) A summary of cleavage of oligonucleotide with hAAG/Endo VIII, hAAG/chemical base and hAAG/alternative chemical base.

f) Oligonucleotides used for study of the cleavage step.

FIG. 22. Version 1 Chemistry with Helper Strand—Ligation.

a) Scheme showing ligation of hybridized polynucleotide strands in the presence of a helper strand. Ligation step highlighted in dashed box.

b) Gel showing ligation of oligonucleotides with Quick T4 DNA ligase at room temperature (24° C.) in the presence of a helper strand. Lane 1 contained a mixture of the 36mers TAMRA single stranded oligos and 18mers TAMRA single stranded oligos. These oligos served reference bands. In lane 2 there was an observable ligation product of expected band size 36mers after 20 minutes.

c) Gel showing ligation of oligonucleotides with Quick T4 DNA ligase at room temperature (24° C.) after overnight incubation in the presence of a helper strand. Lane 1 contained a mixture of the 36mers TAMRA single stranded oligos and 18mers TAMRA single stranded oligos. These oligos served as reference bands. In lane 2 there was an observable completely ligated product of expected band size of 36mers.

d) Oligonucleotides used for study of the ligation step.

FIG. 23. Version 2 Chemistry with Helper Strand—Incorporation.

a) Scheme showing incorporation step highlighted in orange dashed box

b) Gel showing results of incorporation of 3′-O-modified-dTTPs by Therminator IX DNA polymerase at 27° C. Lane 1: Starting material. Lane 2: Incorporation after 1 minute, conversion 5%. Lane 3: Incorporation after 2 minutes, conversion 10%. Lane 4: Incorporation after 5 minutes, conversion 20%. Lane 5: Incorporation after 10 minutes, conversion 30%. Lane 6: Incorporation after 20 minutes, conversion 35%.

c) The Figure depicts a gel showing results of incorporation of 3′-O-modified-dTTPs by Therminator IX DNA polymerase at 37° C. Lane 1: Starting material. Lane 2: Incorporation after 1 minute, conversion 30%. Lane 3: Incorporation after 2 minutes, conversion 60%. Lane 4: Incorporation after 5 minutes, conversion 90%. Lane 5: Incorporation after 10 minutes, conversion 90%. Lane 6: Incorporation after 20 minutes, conversion 90%.

d) Gel showing results of incorporation of 3′-O-modified-dTTPs by Therminator IX DNA polymerase at 47° C. Lane 1: Starting material. Lane 2: Incorporation after 1 minute, conversion 30%. Lane 3: Incorporation after 2 minutes, conversion 65%. Lane 4: Incorporation after 5 minutes, conversion 90%. Lane 5: Incorporation after 10 minutes, conversion 90%. Lane 6: Incorporation after 20 minutes, conversion 90%.

e) Gel showing results of incorporation of 3′-O-modified-dTTPs by Therminator IX DNA polymerase at 27° C. Lane 1: Starting material. Lane 2: Incorporation after 1 minute, conversion 70%. Lane 3: Incorporation after 2 minutes, conversion 85%. Lane 4: Incorporation after 5 minutes, conversion 92%. Lane 5: Incorporation after 10 minutes, conversion 96%. Lane 6: Incorporation after 20 minutes, conversion 96%.

f) Gel showing results of incorporation of 3′-O-modified-dTTPs by Therminator IX DNA polymerase at 37° C. Lane 1: Starting material. Lane 2: Incorporation after 1 minute, conversion 85%. Lane 3: Incorporation after 2 minutes, conversion 95%. Lane 4: Incorporation after 5 minutes, conversion 96%. Lane 5: Incorporation after 10 minutes, conversion 96%. Lane 6: Incorporation after 20 minutes, conversion 96%.

g) Gel showing results of incorporation of 3′-O-modified-dTTPs by Therminator IX DNA polymerase at 47° C. Lane 1: Starting material. Lane 2: Incorporation after 1 minute, conversion 85%. Lane 3: Incorporation after 2 minutes, conversion 90%. Lane 4: Incorporation after 5 minutes, conversion 96%. Lane 5: Incorporation after 10 minutes, conversion 96%. Lane 6: Incorporation after 20 minutes, conversion 96%.

h) Summary of incorporation of 3′-O-azidomethyl-dTTP at various temperatures and presence of Mn²⁺ ions.

i) Gel showing results of incorporation of 3′-O-modified-dNTPs opposite complementary base by Therminator IX DNA polymerase in presence of Mn²⁺ at 37° C. Lane 1: Starting material. Lane 2: Incorporation of 3′-O-azidomethyl-dTTP for 5 minutes. Lane 3: Incorporation of 3′-O-azidomethyl-dATP for 5 minutes. Lane 4: Incorporation of 3′-O-azidomethyl-dCTP for 5 minutes. Lane 5: Incorporation of 3′-O-azidomethyl-dGTP for 5 minutes.

j) Oligonucleotides used for study of the incorporation step.

FIG. 24. Version 2 Chemistry with Helper Strand—Cleavage.

a) Scheme showing cleavage of hybridized polynucleotide strand in the presence of a helper strand. Cleavage step is highlighted in orange dashed box.

b) Gel shows cleavage of Oligonucleotide with Endo V at 37° C. Lane 1. Starting oligonucleotide. Lane 2 which was a positive control that contained both full length strands showed a yield of cleaved to uncleaved DNA ratio of 80%:20%. Lane 3 which included the cleavage reaction without a helper strand showed a much higher yield of cleaved DNA of >99%. Lane 4 which included the cleavage reaction with a helper strand also showed a DNA cleavage yield of >99%.

c) A summary of cleavage study with Endonuclease V.

d) Oligonucleotides used for study of the cleavage step.

FIG. 25. Version 2 Chemistry with Helper Strand—Ligation.

a) Scheme showing ligation of hybridized polynucleotide strands in the absence of a helper strand. Ligation step highlighted in orange dashed box.

b) Oligonucleotides for study of the ligation step.

FIG. 26. Version 2 Chemistry with Helper Strand—Deprotection.

a) Scheme showing deprotection step highlighted in orange dashed box.

b) The Figure depicts a gel showing results of 3′-O-azidomethyl group deprotection by 50 mM TCEP after incorporation of 3′-O-azidomethyl-dTTP. Lane 1: Starting primer Lane 2: Incorporation of 3′-O-azidomethyl-dTTPs in presence Mn²⁺. Lane 3: Extension of the product in lane 2 by addition of all natural dNTPs. Lane 4: Deprotection of the product (0.5 μM) in lane 2 by 50 mM TCEP. Lane 5: Extension of the product in lane 4 by addition of all natural dNTPs.

c) The Figure depicts a gel showing results of 3′-O-azidomethyl group deprotection by 300 mM TCEP after incorporation of 3′-O-azidomethyl-dTTP. Lane 1: Starting primer. Lane 2: Incorporation of 3-O-azidomethyl-dTTPs in presence Mn²⁺. Lane 3: Extension of the product in lane 2 by addition of all natural dNTPs. Lane 4: Deprotection of the product (0.5 μM) in lane 2 by 300 mM TCEP. Lane 5: Extension of the product in lane 4 by addition of all natural dNTPs.

d) The Figure depicts a gel showing results of 3′-O-azidomethyl group deprotection by 50 mM TCEP after incorporation of 3′-O-azidomethyl-dCTP. Lane 1: Starting primer. Lane 2: Incorporation of 3-O-azidomethyl-dCTPs in presence Mn²⁺. Lane 3: Extension of the product in lane 2 by addition of all natural dNTPs. Lane 4: Deprotection of the product (0.5 μM) in lane 2 by 300 mM TCEP. Lane 5: Extension of the product in lane 4 by addition of all natural dNTPs.

e) The Figure depicts a gel showing results of 3′-O-azidomethyl group deprotection by 300 mM TCEP after incorporation of 3′-O-azidomethyl-dCTP. Lane 1: Starting primer Lane 2: Incorporation of 3-O-azidomethyl-dCTPs in presence Mn²⁺. Lane 3: Extension of the product in lane 1 by addition of all natural dNTPs. Lane 4: Deprotection of the product (0.5 μM) in lane 1 by 300 mM TCEP. Lane 5: Extension of the product in lane 3 by addition of all natural dNTPs.

f). The Figure depicts a gel showing results of 3′-O-azidomethyl group deprotection by 300 mM TCEP after incorporation of 3′-O-azidomethyl-dATP.

Lane 1: Starting primer

Lane 2: Incorporation of 3-O-azidomethyl-dATPs in presence Mn²⁺. Lane 3: Extension of the product in lane 2 by addition of all natural dNTPs. Lane 4: Deprotection of the product (0.5 μM) in lane 2 by 300 mM TCEP. Lane 5: Extension of the product in lane 4 by addition of all natural dNTPs.

g) The Figure depicts a gel showing results of 3′-O-azidomethyl group deprotection by 300 mM TCEP after incorporation of 3′-O-azidomethyl-dGTP. Lane 1: Starting primer. Lane 2: Incorporation of 3-O-azidomethyl-dGTPs in presence Mn²⁺. Lane 3: Extension of the product in lane 2 by addition of all natural dNTPs. Lane 4: Deprotection of the product (0.5 μM) in lane 2 by 300 mM TCEP. Lane 5: Extension of the product in lane 4 by addition of all natural dNTPs.

h) Efficiency of deprotection by TCEP on 0.2 μM DNA.

i) Oligonucleotides used for study of the cleavage step.

FIG. 27. Version 2 Chemistry with Double Hairpin Model—Incorporation.

a) Scheme showing incorporation step highlighted in dashed box.

b) Evaluation of DNA polymerases for incorporation of 3′-O-modified-dTTPs opposite its natural counterpart. The Figure depicts a gel showing results of incorporation of 3′-O-modified-dTTPs by Therminator IX DNA polymerase at 37° C. Lane 1: Starting material. Lane 2: Incorporation of natural dNTP mix. Lane 3: Incorporation of 3′-O-azidomethyl-dTTP by Therminator IX DNA polymerase. Lane 4: Extension of the product in lane 3 by addition of all natural dNTPs.

c) Evaluation of DNA polymerases for incorporation of 3′-O-modified-dTTPs opposite its natural counterpart. Oligonucleotides applicable for study of the incorporation step.

FIG. 28. Version 2 Chemistry with Double Hairpin Model—Cleavage.

a) Scheme showing cleavage of a hairpin Oligonucleotide. Cleavage step is highlighted in dashed box.

b) Gel showing cleavage of Hairpin Oligonucleotide with Endo V at 37° C. Lane 1. Starting hairpin oligonucleotide. Lane 2 which was the cleaved hairpin oligonucleotide after 5 minutes showed a high yield of digested DNA with a ratio of ˜98%. Lane 3 which was the cleaved hairpin oligonucleotide after 10 minutes showed a high yield of digested DNA with a ratio of ˜99%. Lane 4 which was the cleaved hairpin oligonucleotide after 30 minutes showed a high yield of digested DNA with a ratio of ˜99% and in lane 5 which was the cleaved hairpin oligonucleotide after 1 hr showed a high yield of digested DNA with a ratio of ˜99%.

c) Oligonucleotides used for study of the cleavage step.

FIG. 29. Version 2 Chemistry with Double Hairpin Model—Ligation.

a) Scheme showing ligation of hybridized hairpins. Ligation step highlighted in dashed box.

b) The gel shows ligation of Hairpin Oligonucleotides with Blunt/TA DNA ligase at room temperature (24° C.) in the presence of a helper strand. Lane 1 contained a starting hairpin Oligonucleotide. Lane 2 which was the ligated hairpin oligonucleotide after 1 minute showed a high yield of ligated DNA product with a ratio of ˜85%. Lane 3 which was the ligated hairpin oligonucleotide after 2 minutes showed a high yield of digested DNA with a ratio of ˜85%. Lane 4 which was the ligated hairpin oligonucleotide after 3 minutes showed a high yield of ligated DNA product with a ratio of ˜85%. Lane 5 which was the ligated hairpin oligonucleotide after 4 minutes showed a high yield of ligated DNA product with a ratio of ˜>85%.

c) Hairpin Oligonucleotides used for study of the Ligation step.

FIG. 30. Version 2 Chemistry—Complete Cycle on Double Hairpin Model.

a) Scheme showing full cycle involving enzymatic incorporation, cleavage, ligation and deprotection steps.

b) Evaluation of DNA polymerases for incorporation of 3′-O-modified-dTTPs opposite its natural counterpart. The Figure depicts a gel showing results of incorporation of 3′-O-modified-dTTPs by Therminator IX DNA polymerase at 37° C. Lane 1: Starting material. Lane 2: Incorporation of 3′-O-azidomethyl-dTTP by Therminator IX DNA polymerase. Lane 3: Extension of the product in lane 2 by addition of all natural dNTPs. Lane 4: Cleavage of the product in lane 2 by Endonuclease V. Lane 5: Ligation of the product in lane 4 by blunt TA ligase kit.

c) Oligonucleotides applicable for study of the incorporation step.

FIG. 31. Version 2 Chemistry—Complete Cycle on Single Hairpin Model Using Helper Strand

a) Scheme showing full cycle involving enzymatic incorporation, cleavage, ligation and deprotection steps.

b) Oligonucleotides applicable for study of the incorporation step.

FIG. 32. Version 3 Chemistry—Complete Cycle on Double-Hairpin Model.

a) Scheme showing full cycle involving enzymatic incorporation, cleavage, ligation and deprotection steps.

b) Oligonucleotides applicable for study of the incorporation step.

FIG. 33. Version 2 Chemistry—Complete Two-Cycle on Double-Hairpin Model

a) Scheme showing the first full cycle involving enzymatic incorporation, deprotection, cleavage and ligation steps.

b) Scheme showing the second full cycle, following the first full cycle, involving enzymatic incorporation, deprotection, cleavage and ligation steps.

c) The Figure depicts a gel showing full two-cycle experiment comprising: incorporation, deprotection, cleavage and ligation steps.

Lane 1. Starting material.
Lane 2. Extension of starting material with natural dNTPs.
Lane 3. Incorporation of 3′-O-azidomethyl-dTTP by Therminator IX DNA polymerase.
Lane 4. Extension of the product in lane 3 by addition of all natural dNTPs.
Lane 5. Deprotection of the product in lane 3 by TCEP.
Lane 6. Extension of the product in lane 5 by addition of all natural dNTPs.
Lane 7. Cleavage of the product in lane 5 by Endonuclease V.
Lane 8. Ligation of the product in lane 7 by blunt TA ligase kit.
Lane 9. Cleavage of the product in lane 8 by Lambda exonuclease.
Lane 10. Starting material for second cycle—the same material as in lane 9.
Lane 11. Incorporation of 3′-O-azidomethyl-dTTP by Therminator IX DNA polymerase.
Lane 12. Extension of the product in lane 11 by addition of all natural dNTPs.
Lane 13. Deprotection of the product in lane 11 by TCEP.
Lane 14. Extension of the product in lane 13 by addition of all natural dNTPs.
Lane 15. Cleavage of the product in lane 13 by Endonuclease V.
Lane 16. Ligation of the product in lane 15 by blunt TA ligase kit.

d) Oligonucleotides used for study.

FIG. 34.

Example showing a mechanism of release from a scaffold polynucleotide of a polynucleotide of predefined sequence, as synthesised in accordance with the methods described herein.

FIG. 35.

Schematic of an exemplary method for the synthesis of RNA according to the invention. The exemplary method shows synthesis in the absence of a helper strand.

FIG. 36.

Schematic of an exemplary method for the synthesis of RNA according to the invention. The exemplary method shows synthesis in the presence of a helper strand.

FIG. 37.

Schematic of an exemplary method for the synthesis of RNA according to the invention. The exemplary method shows synthesis in the presence of a helper strand.

FIG. 38.

Schematic of the 1st full cycle of an exemplary method for the synthesis of DNA according to synthesis method version 2 with single hairpin model, involving a step of denaturing the helper strand prior to the incorporation step.

FIG. 39.

Schematic of the 2nd full cycle of an exemplary method for the synthesis of DNA according to synthesis method version 2 with single hairpin model, involving a step of denaturing the helper strand prior to the incorporation step.

FIG. 40.

Schematic of the 3rd full cycle of an exemplary method for the synthesis of DNA according to synthesis method version 2 with single hairpin model, involving a step of denaturing the helper strand prior to the incorporation step.

FIG. 41.

Oligonucleotides used in the experiments detailed in Example 9.

FIG. 42.

Gel showing reaction products corresponding to a full three-cycle experiment as detailed in Example 9.

The Figure depicts a gel showing the results of a full three-cycle experiment comprising: incorporation, deblock, cleavage and ligation steps.

Lane 1: Starting material.
Lane 2. Extension of starting material with natural dNTPs
Lane 3: Incorporation of 3′-O-azidomethyl-dTTP by Therminator X DNA polymerase.
Lane 4: Extension of the product in lane 3 by addition of all natural dNTPs.¬
Lane 5: Deblock of the product in lane 3 by TCEP
Lane 6: Extension of the product in lane 5 by addition of all natural dNTPs.¬
Lane 7: Cleavage of the product in lane 5 by Endonuclease V.
Lane 8: Ligation of the product in lane 7 by T3 DNA ligase
Lane 9: Starting material for 2nd cycle—the same material as in lane 9
Lane 10: Extension of the product in lane 9 by addition of all natural dNTPs.
Lane 11: Incorporation of 3′-O-azidomethyl-dTTP by Therminator X DNA polymerase.
Lane 12: Extension of the product in lane 11 by addition of all natural dNTPs.
Lane 13: Deblock of the product in lane 11 by TCEP
Lane 14: Extension of the product in lane 13 by addition of all natural dNTPs.
Lane 15: Cleavage of the product in lane 13 by Endonuclease V
Lane 16: Ligation of the product in lane 15 by T3 DNA ligase
Lane 17: Starting material for 3rd cycle—the same material as in lane 16
Lane 18: Extension of the product in lane 17 by addition of all natural dNTPs.
Lane 19: Incorporation of 3′-O-azidomethyl-dTTP by Therminator X DNA polymerase.
Lane 20: Extension of the product in lane 19 by addition of all natural dNTPs.
Lane 21: Deblock of the product in lane 19 by TCEP
Lane 22: Extension of the product in lane 21 by addition of all natural dNTPs.
Lane 23: Cleavage of the product in lane 21 by Endonuclease V
Lane 24: Ligation of the product in lane 23 by T3 DNA ligase

FIG. 43.

Fluorescence signals from polyacrylamide gel surfaces spiked with different amount of BRAPA exposed to FITC-PEG-SH and FITC-PEG-COOH.

FIG. 44.

Measured fluorescence signals from fluorescein channel on polyacrylamide gel surfaces spiked with different amount of BRAPA that are exposed to FITC-PEG-SH and FITC-PEG-COOH.

FIG. 45.

(a) Shows sequences of hairpin DNA without linker immobilised on different samples.

(b) Shows sequences of hairpin DNA with linker immobilised on different samples.

FIG. 46.

Fluorescence signals from hairpin DNA oligomers with and without linker immobilised onto bromoacetyl functionalised polyacrylamide surfaces.

FIG. 47.

Measured fluorescence from hairpin DNA oligomers with and without linker immobilised onto bromoacetyl functionalised polyacrylamide surfaces.

FIG. 48.

Fluorescence signals from hairpin DNA oligomers with and without linker immobilised onto bromoacetyl functionalised polyacrylamide surfaces following incorporation of triphosphates.

FIG. 49.

Measured fluorescence from hairpin DNA oligomers with and without linker immobilised onto bromoacetyl functionalised polyacrylamide surfaces following incorporation of triphosphates.

FIG. 50.

(a) Experimental overview and outcome for each reaction step as detailed in Example 12.

(b) Oligonucleotides used in the experiments detailed in Example 12.

FIG. 51.

Shows fluorescence signals from hairpin DNA oligomers before and after cleavage reactions (Example 12).

FIG. 52.

Shows measured fluorescence signals from hairpin DNA oligomers before and after cleavage reactions (Example 12).

FIG. 53.

Shows the sequences for the inosine-containing strand and the complimentary ‘helper’ strand for ligation reactions (Example 12).

FIG. 54.

Results relating to fluorescence signals from hairpin DNA oligomers corresponding to the monitoring of ligation reactions (Example 12).

FIG. 55.

Results relating to measured fluorescence from hairpin DNA oligomers corresponding to the monitoring of ligation reactions (Example 12).

FIG. 56.

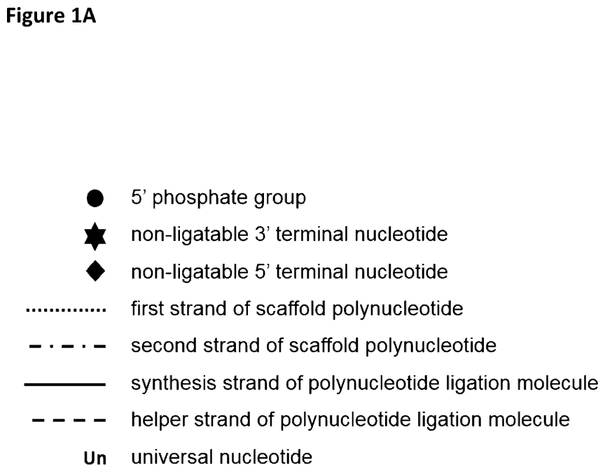

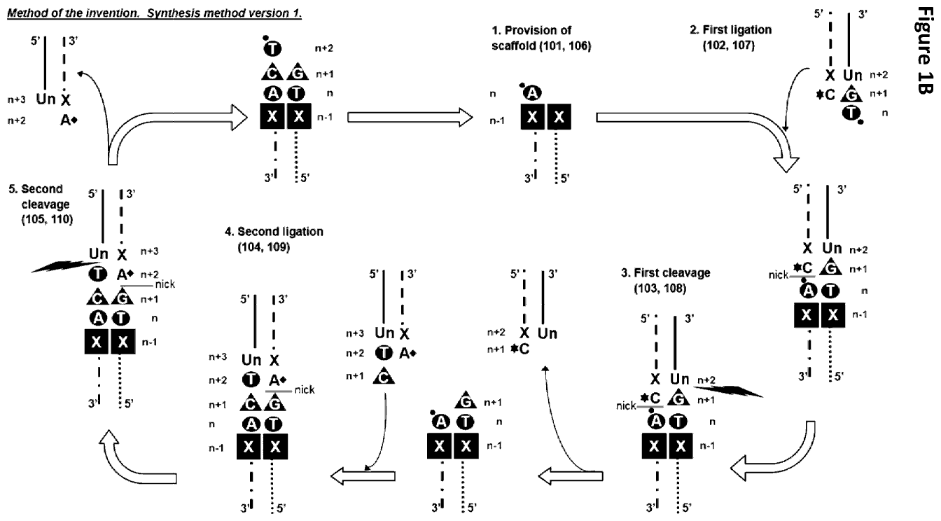

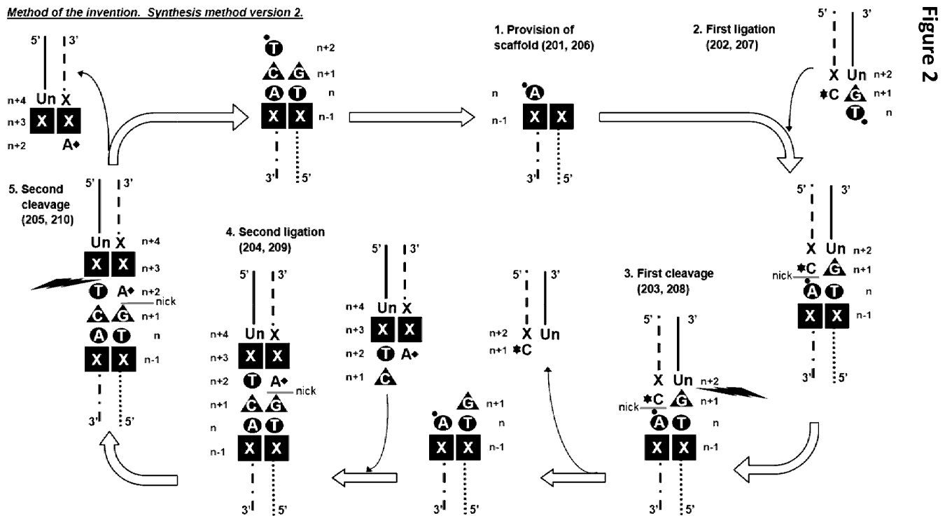

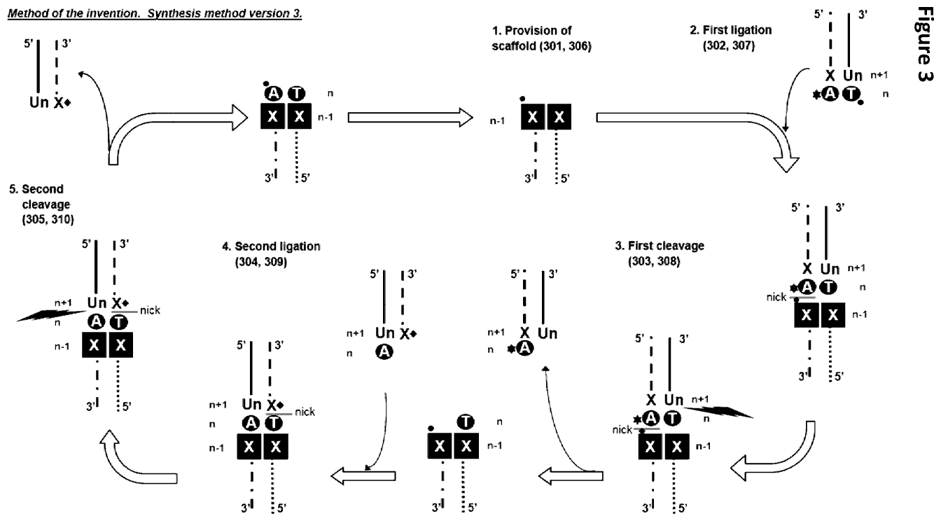

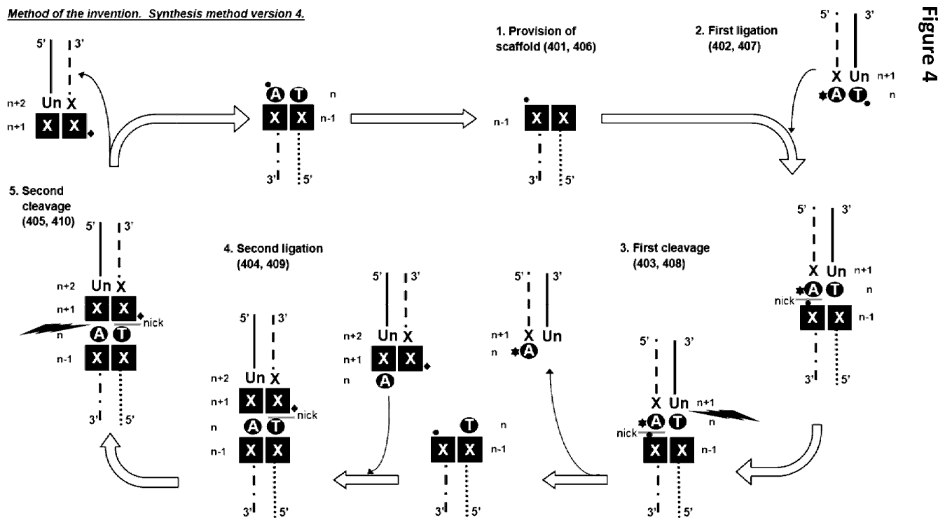

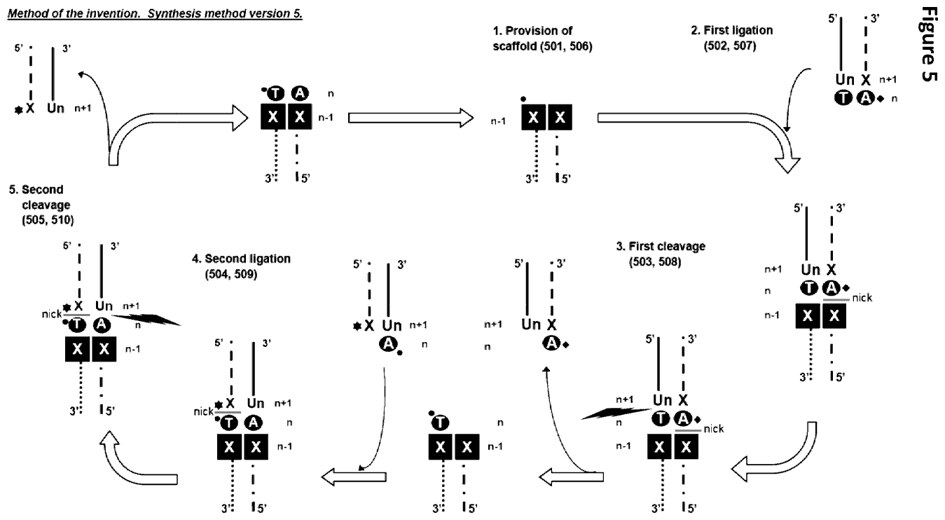

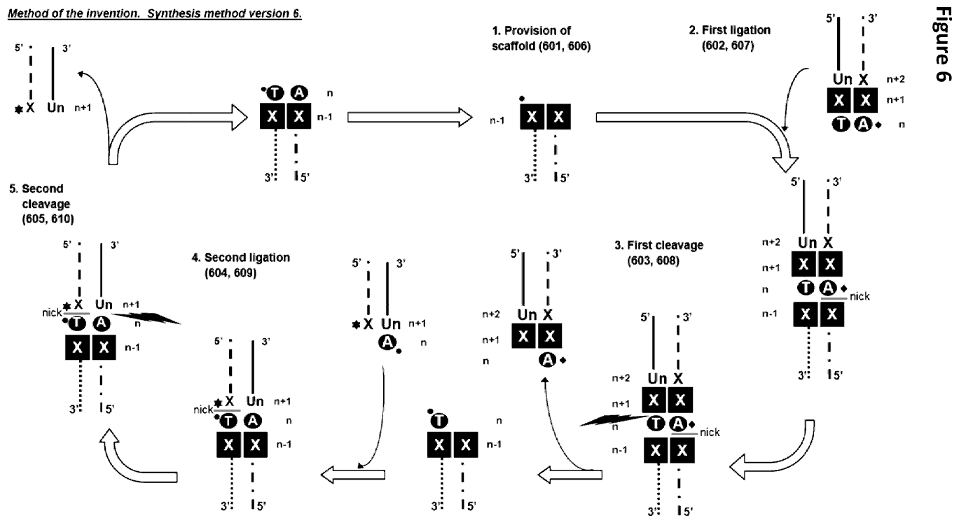

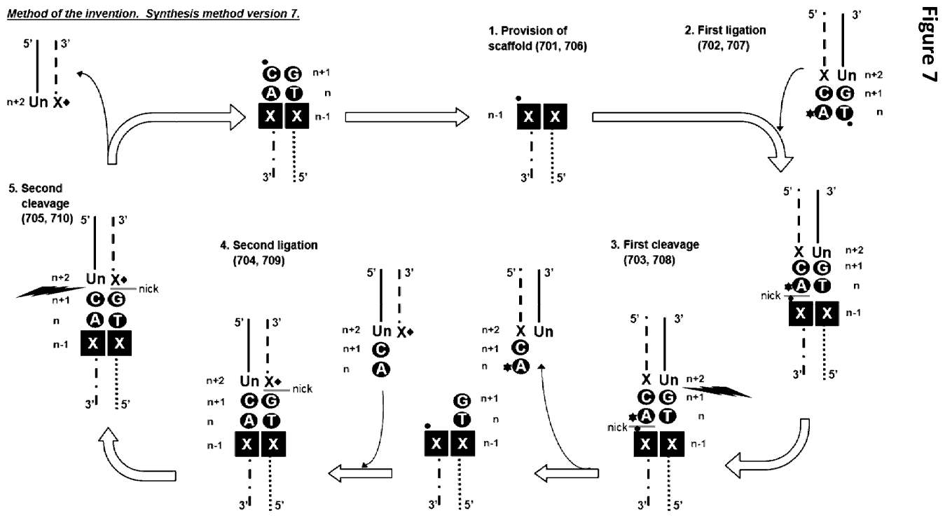

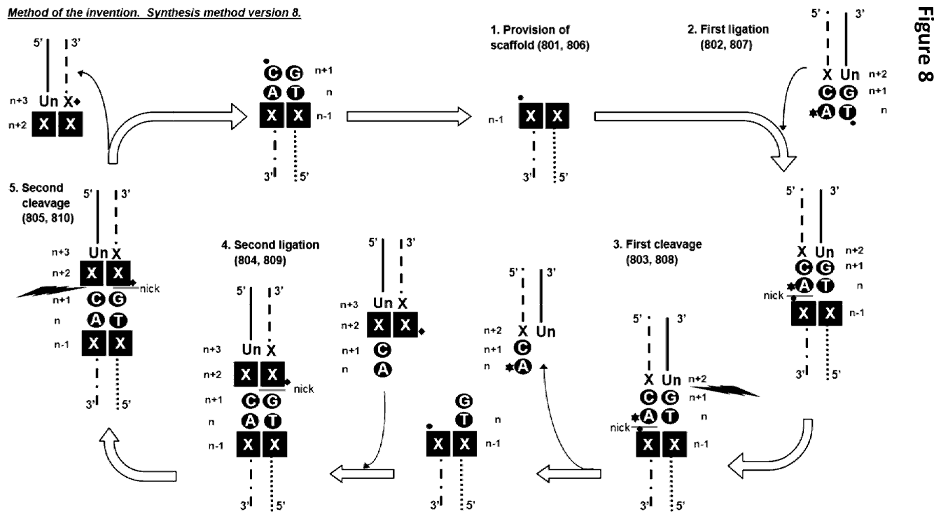

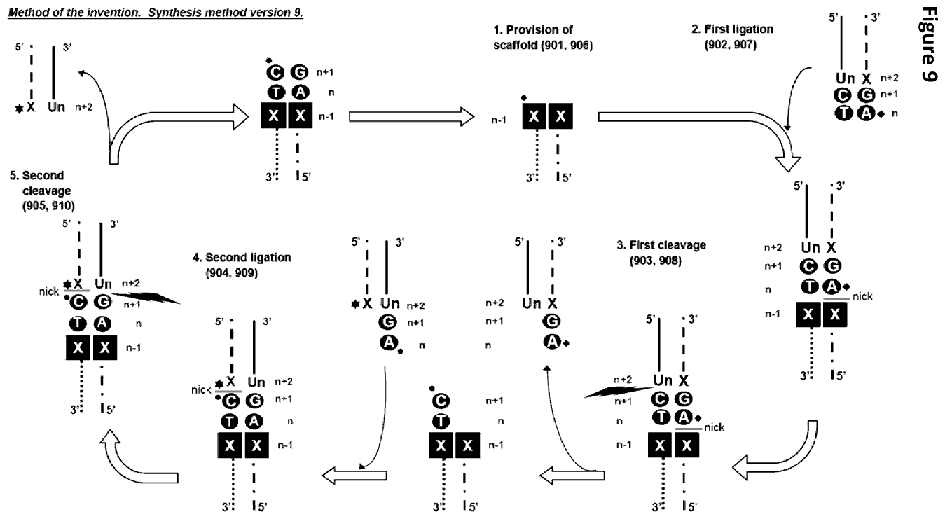

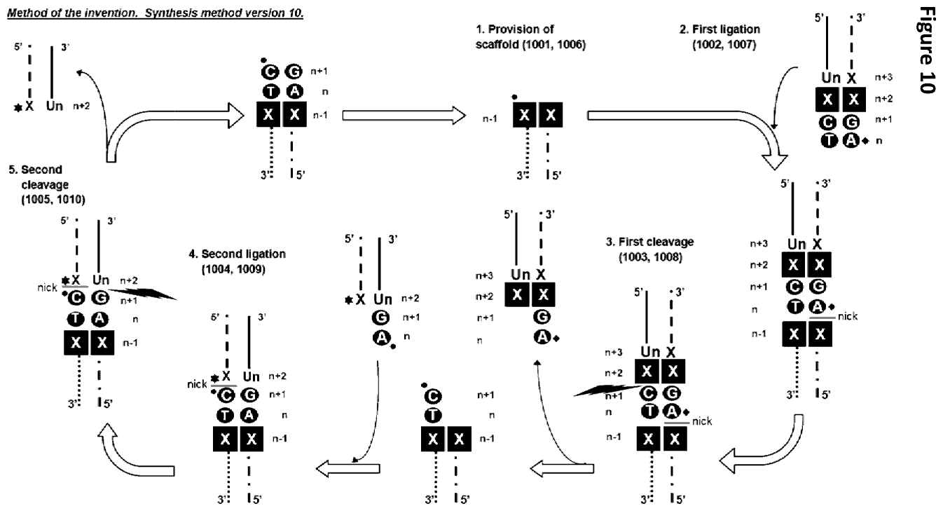

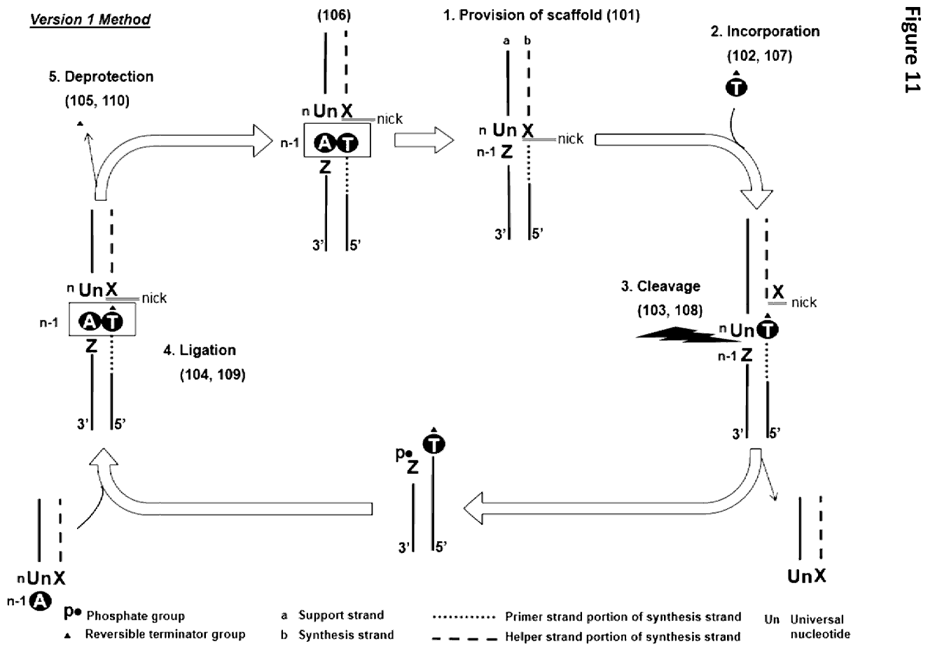

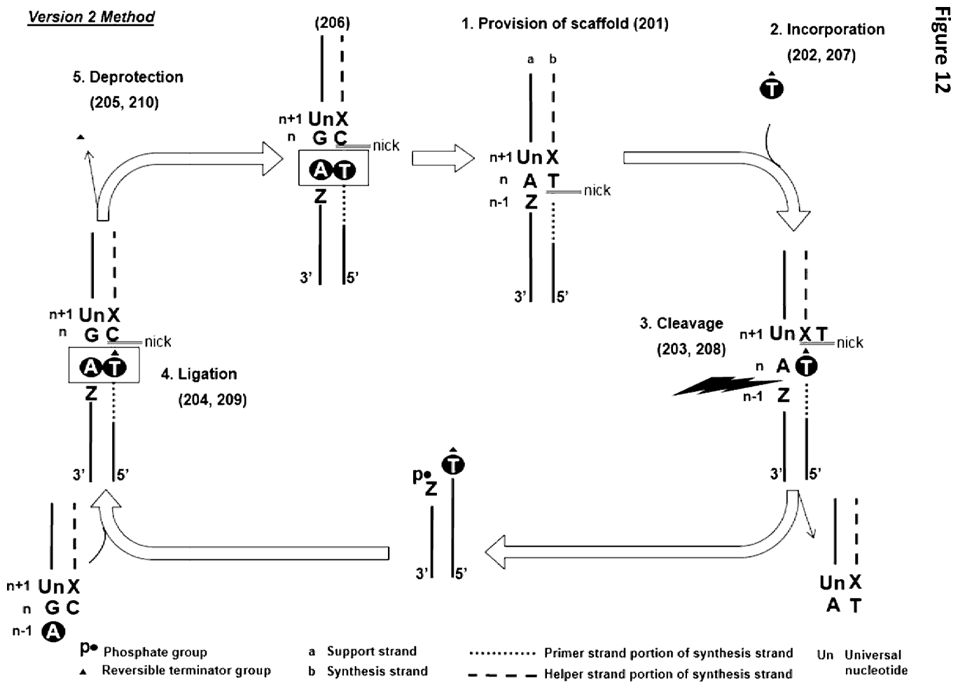

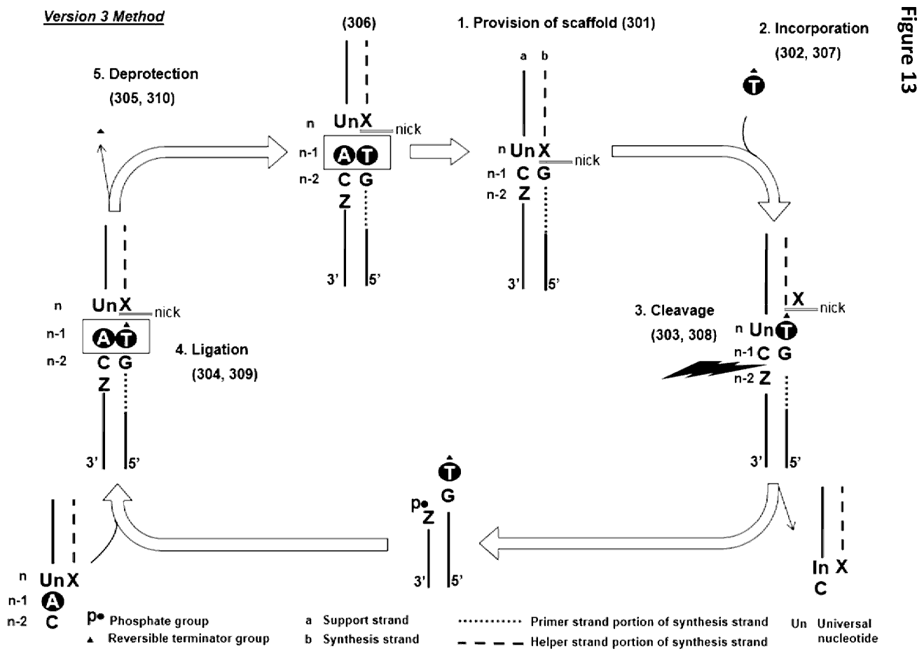

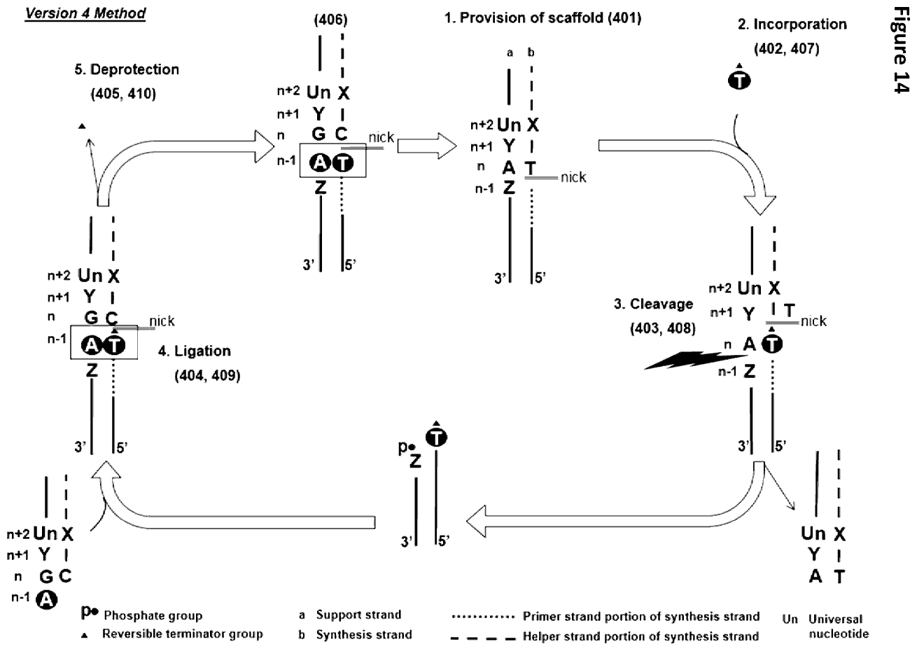

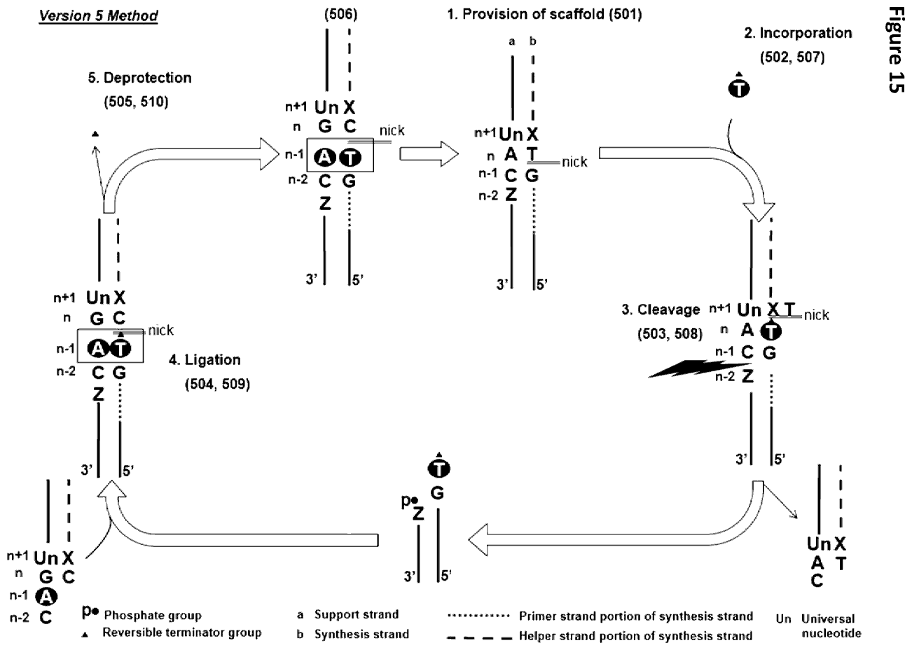

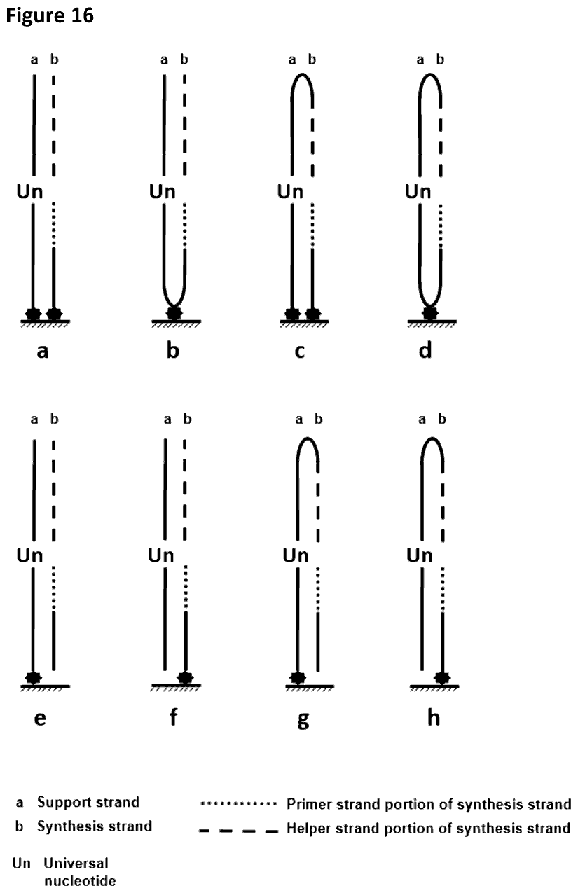

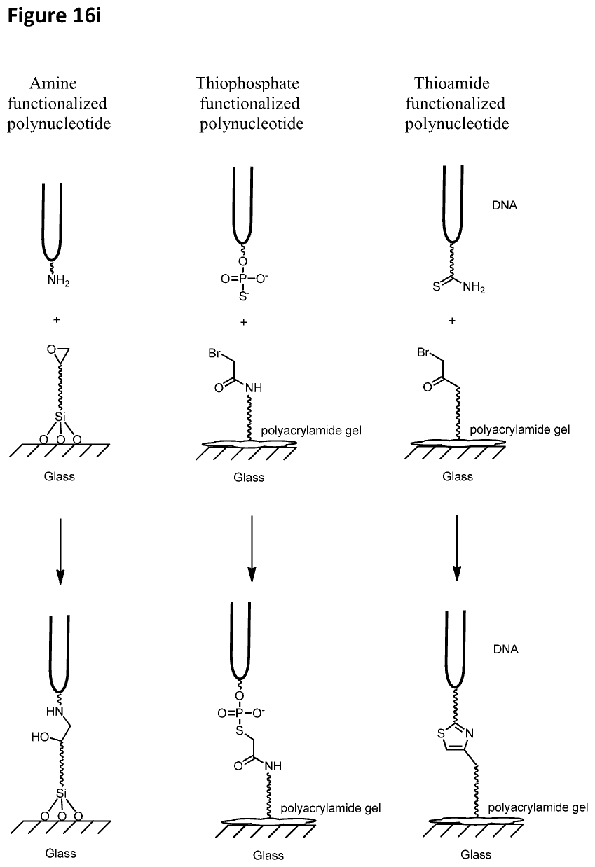

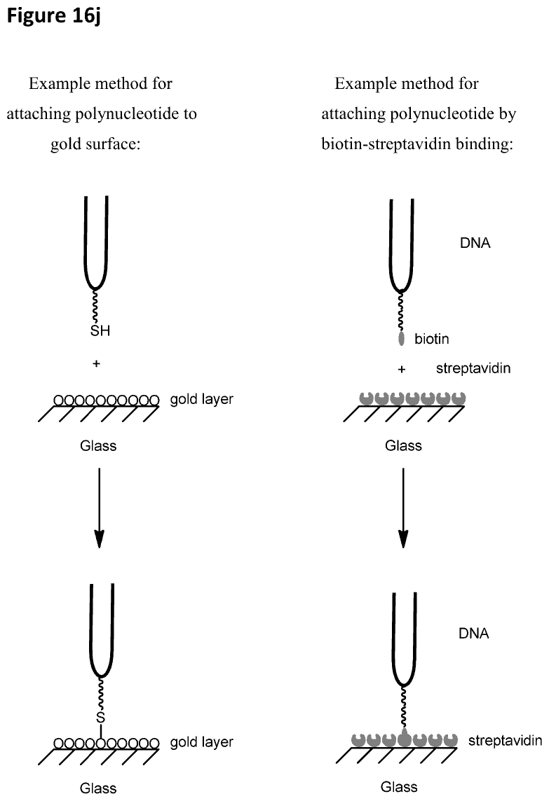

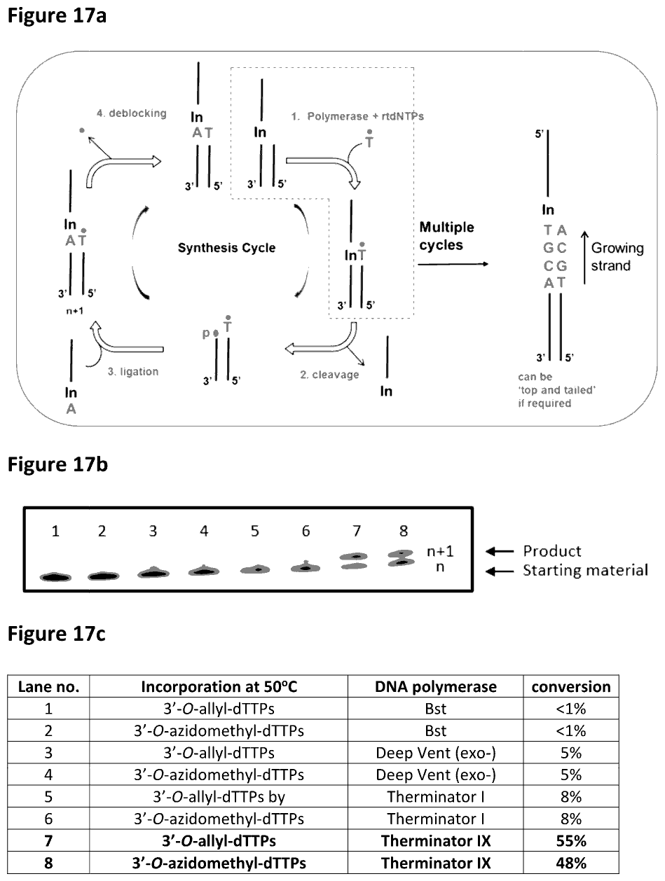

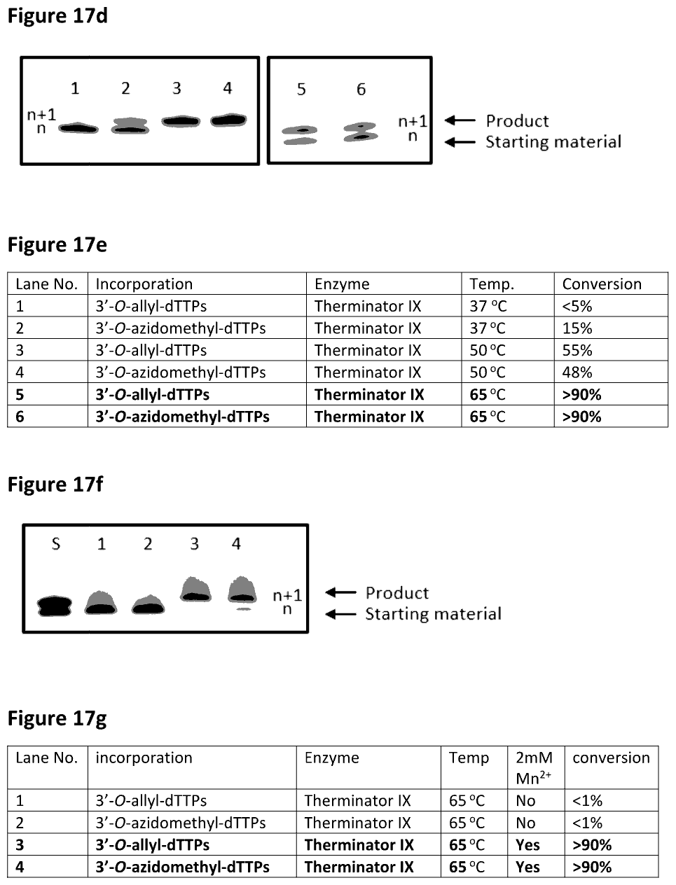

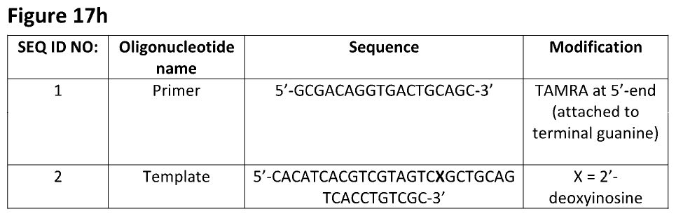

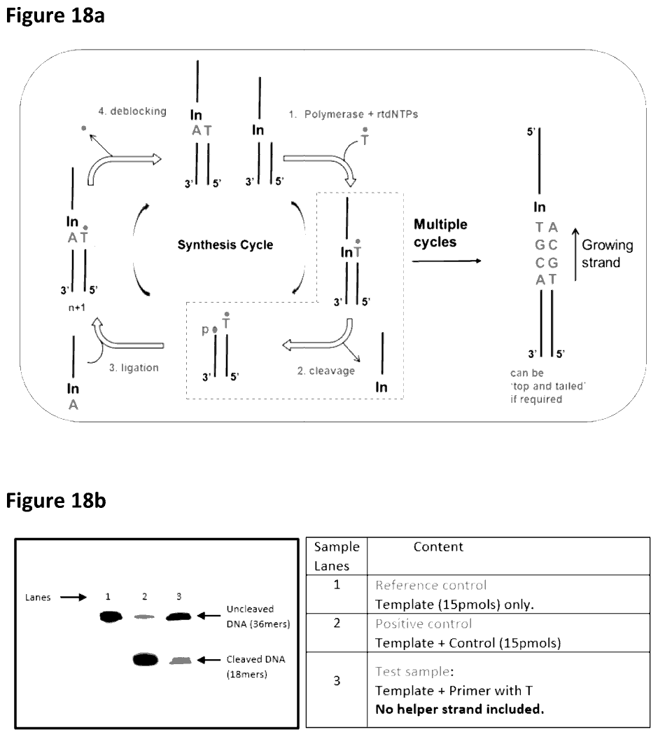

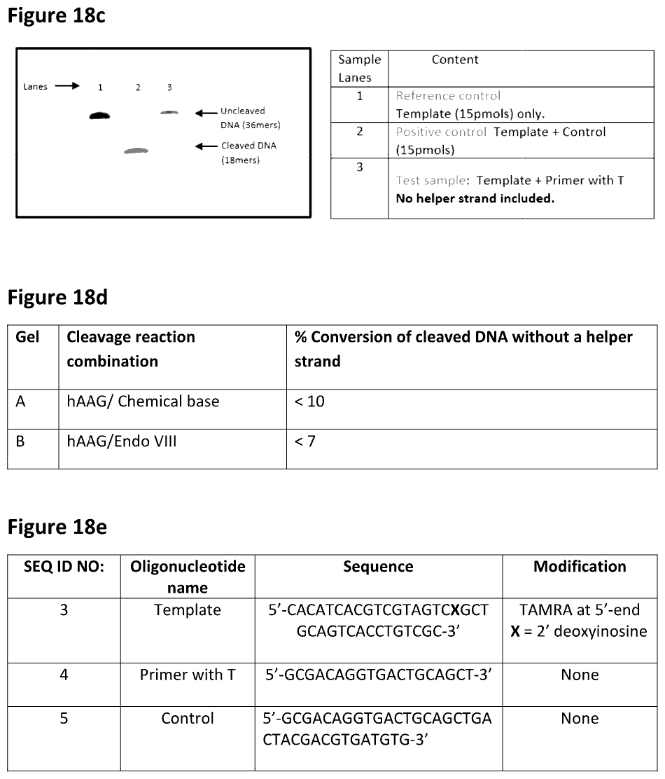

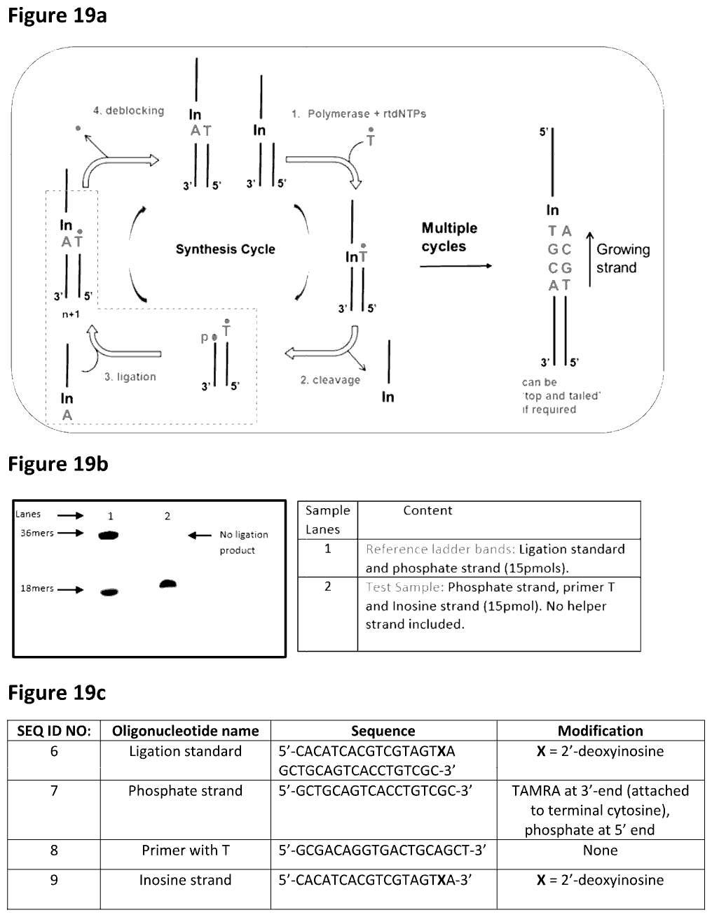

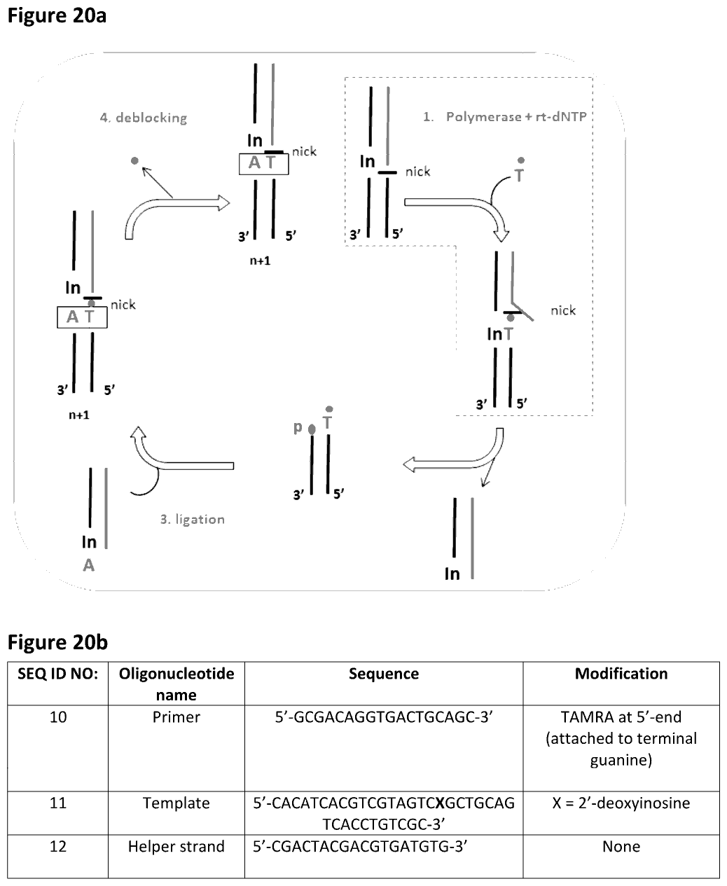

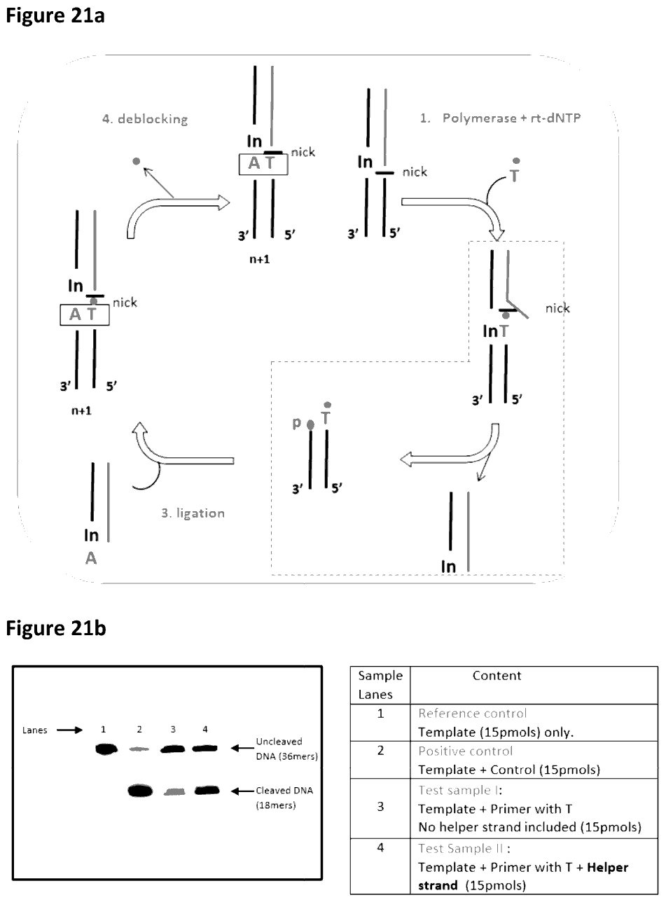

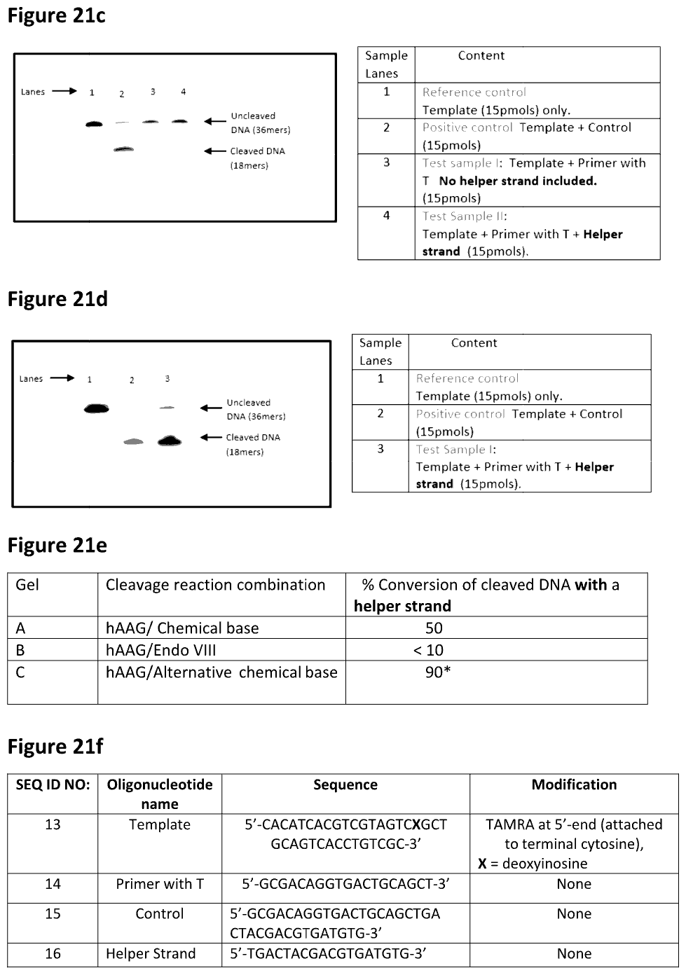

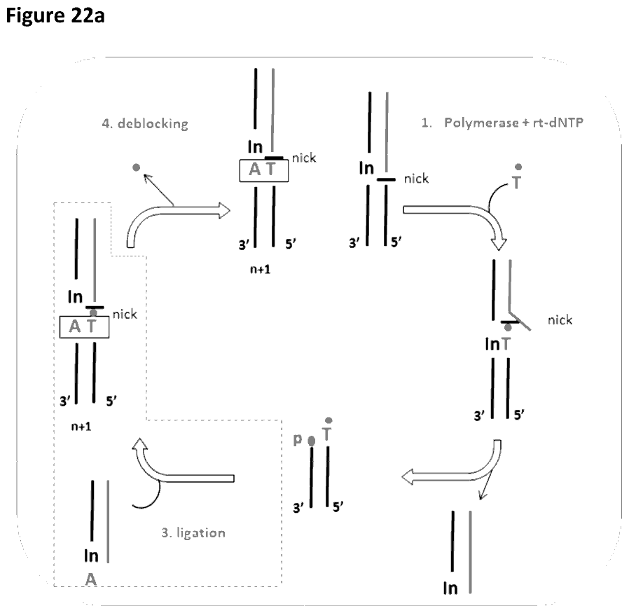

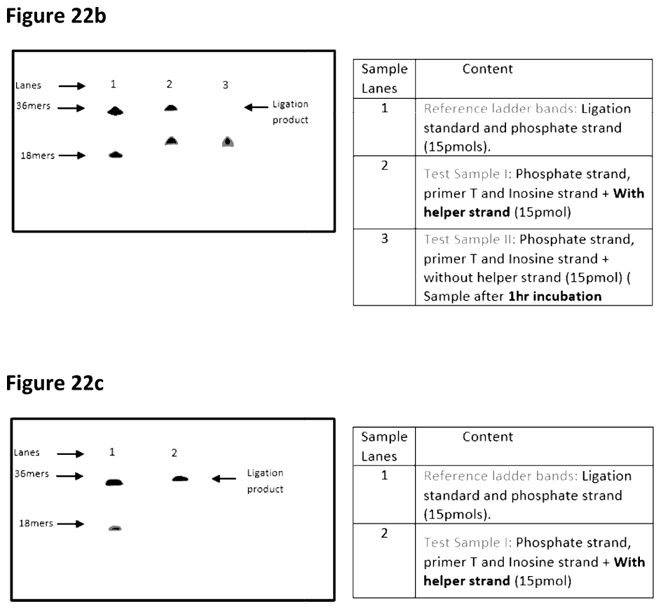

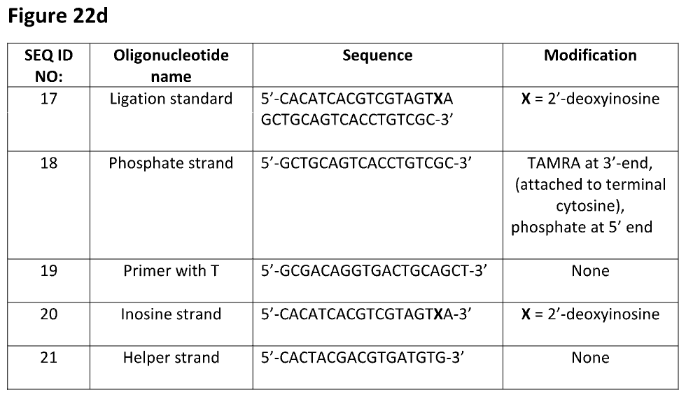

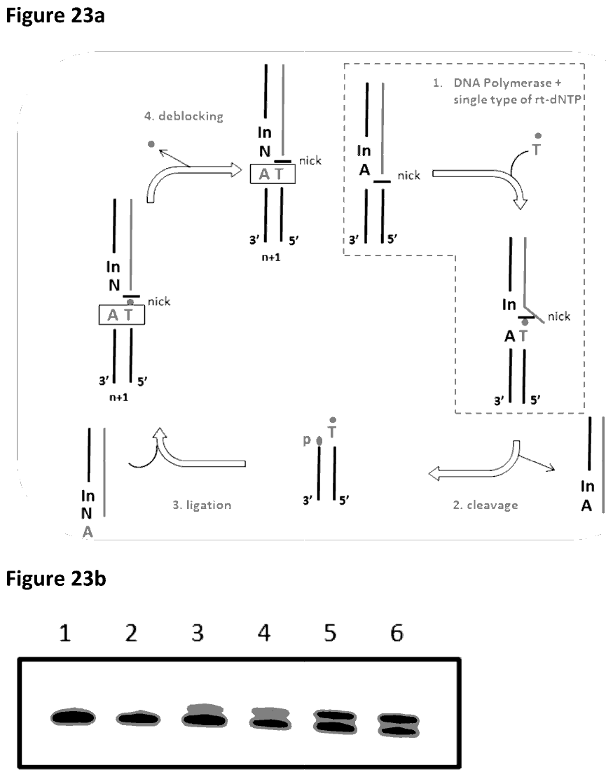

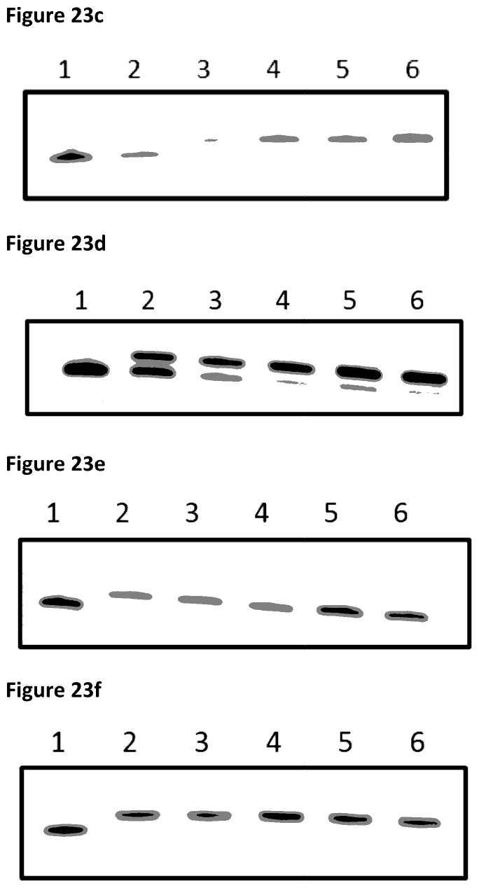

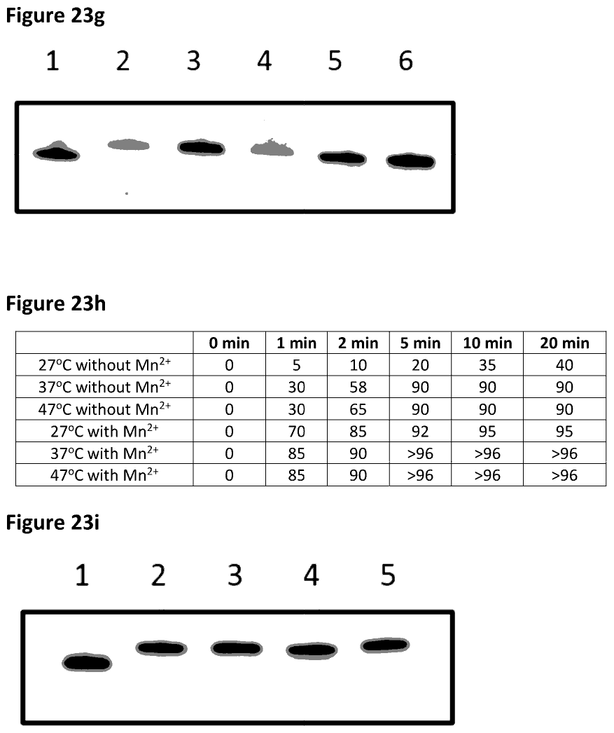

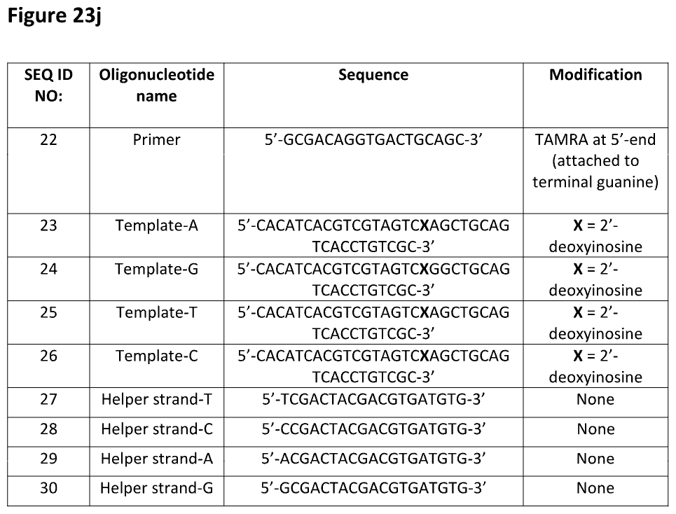

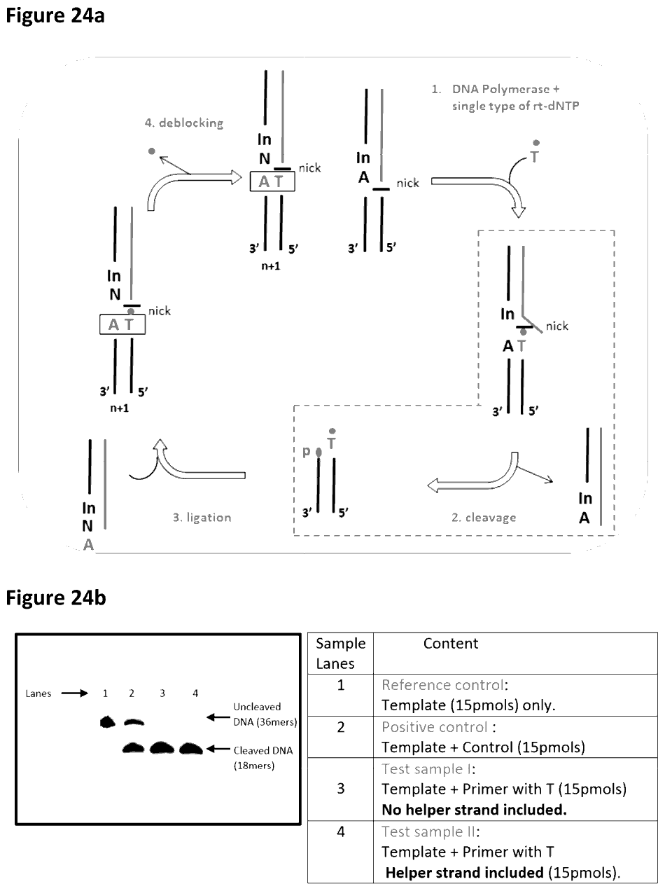

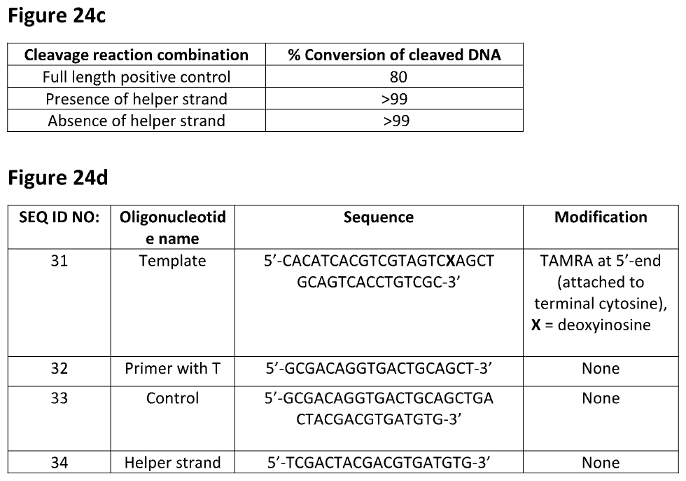

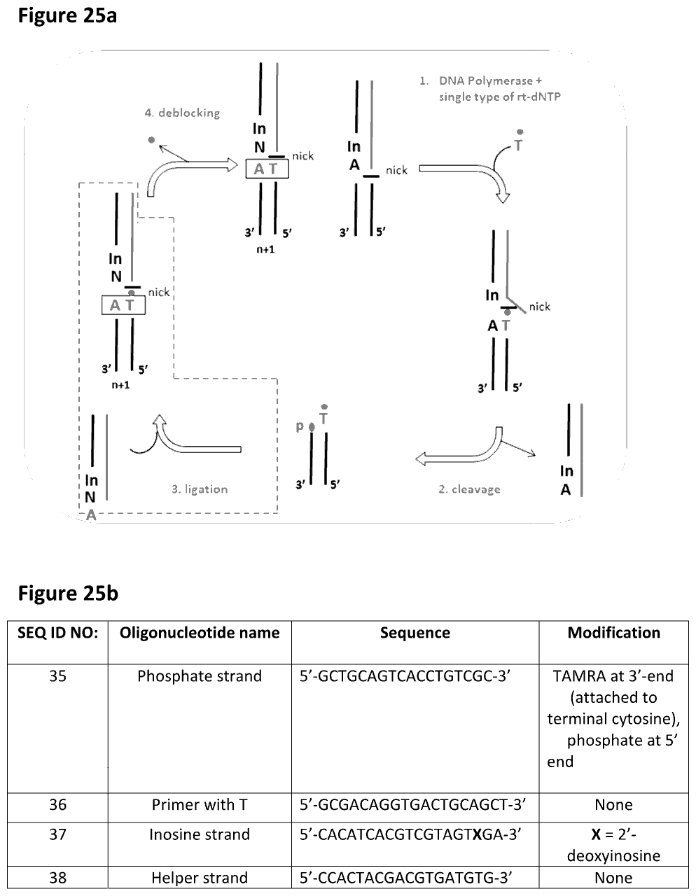

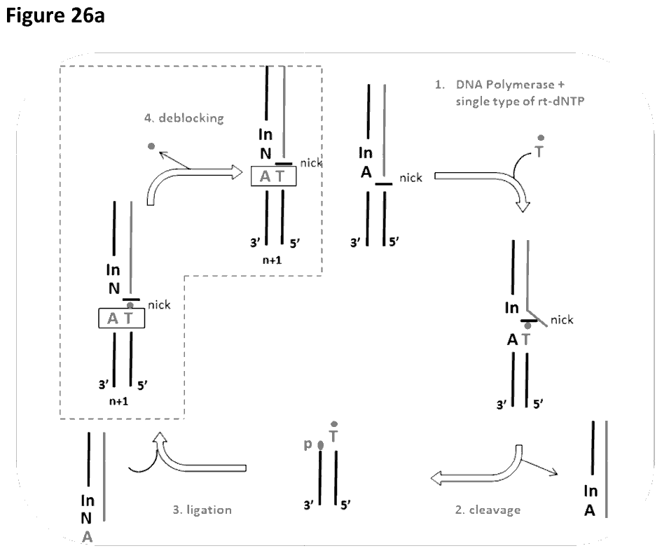

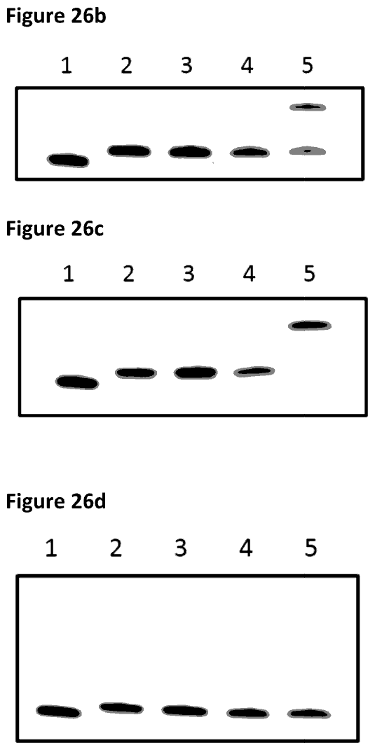

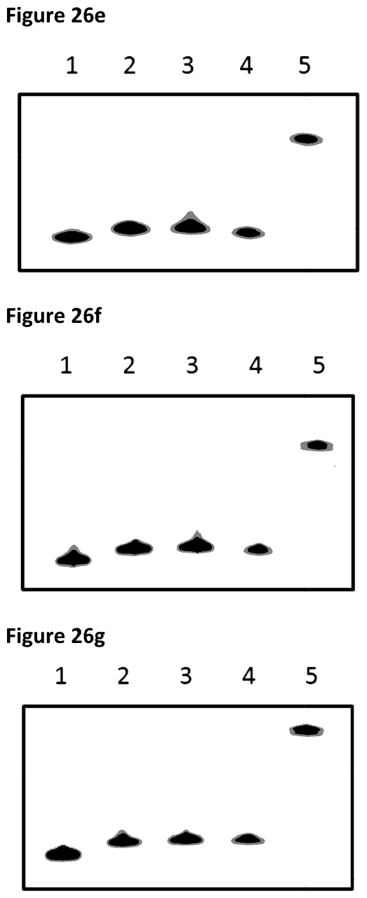

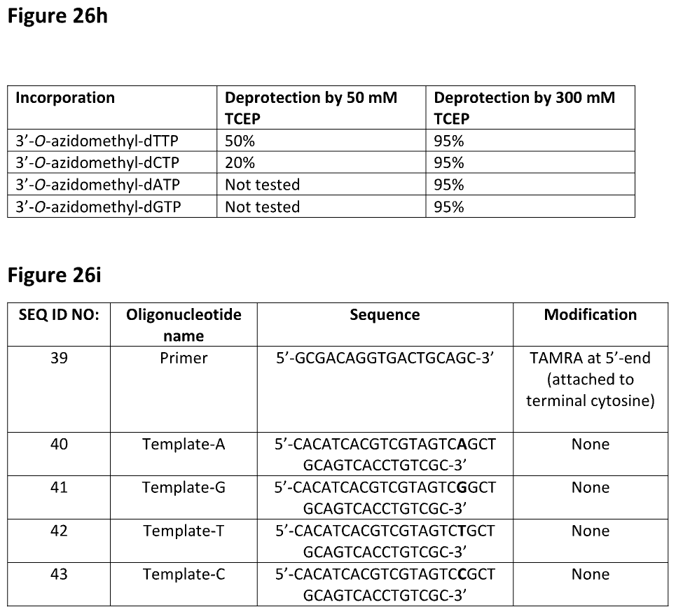

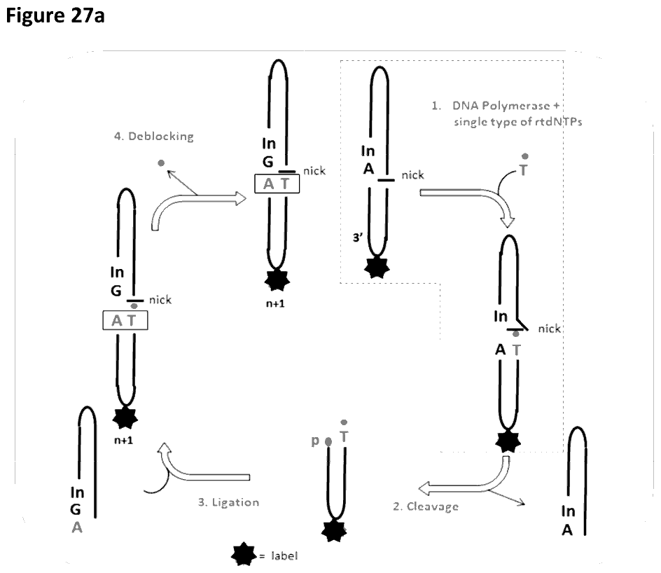

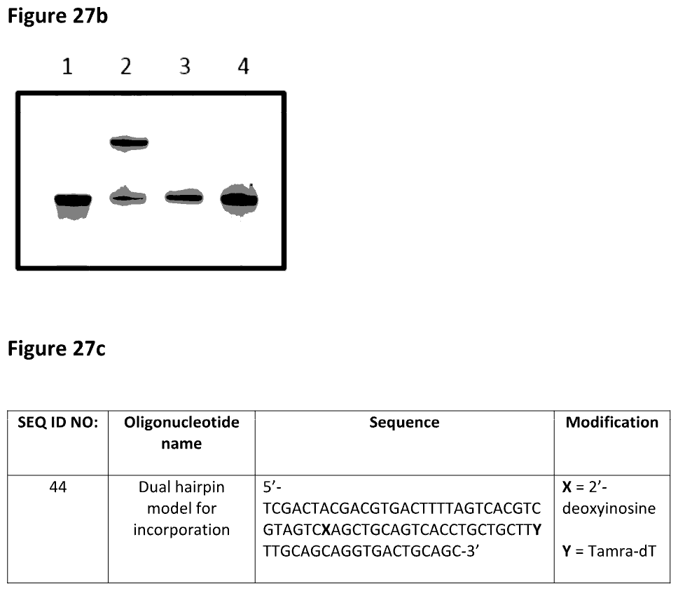

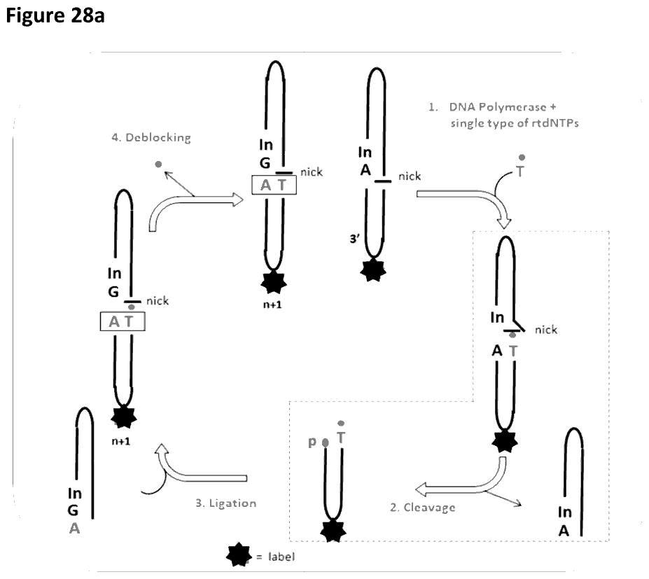

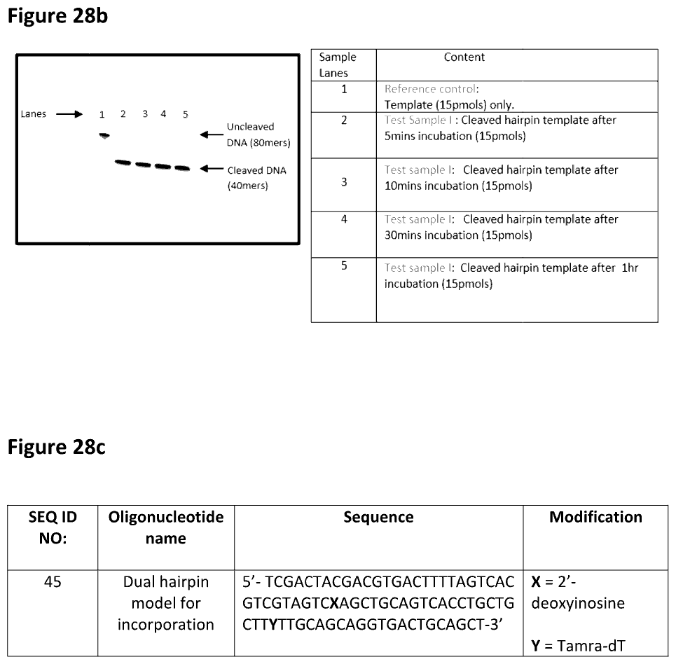

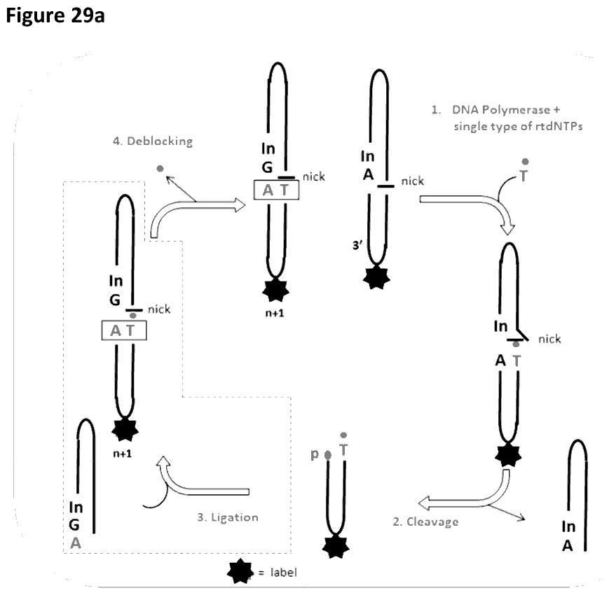

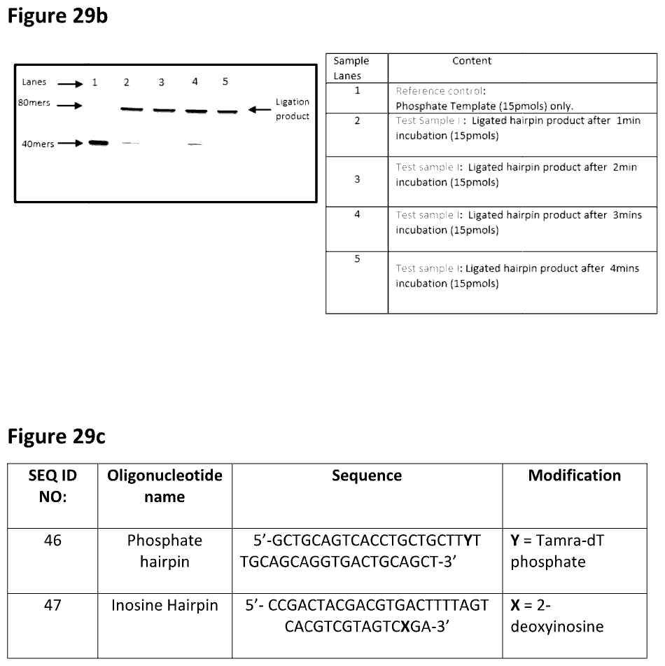

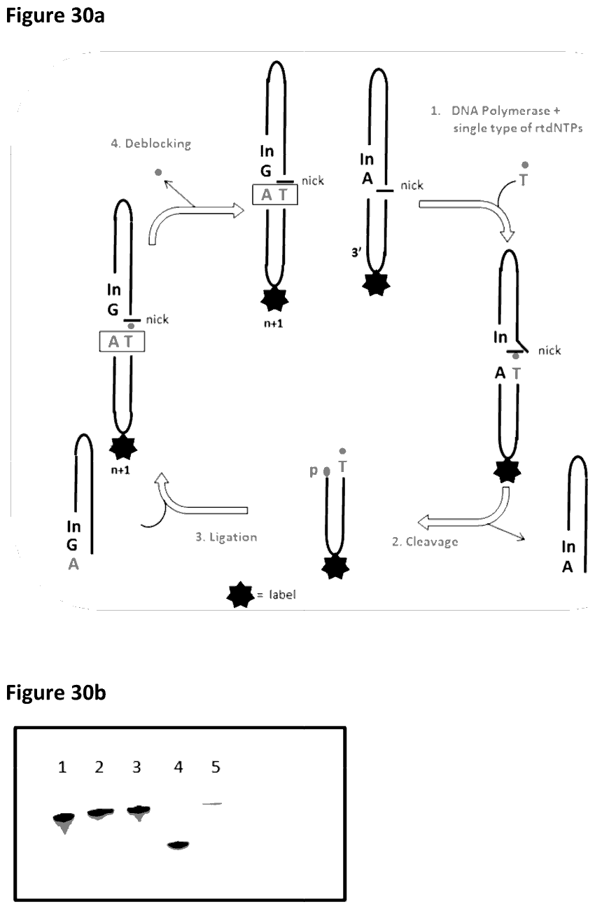

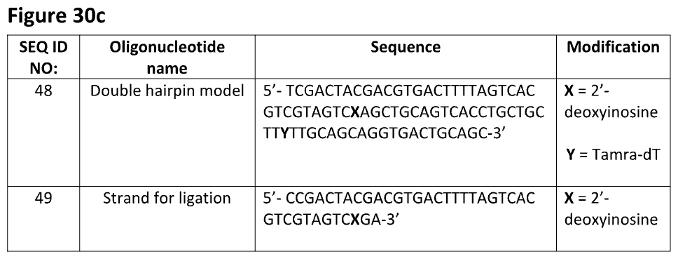

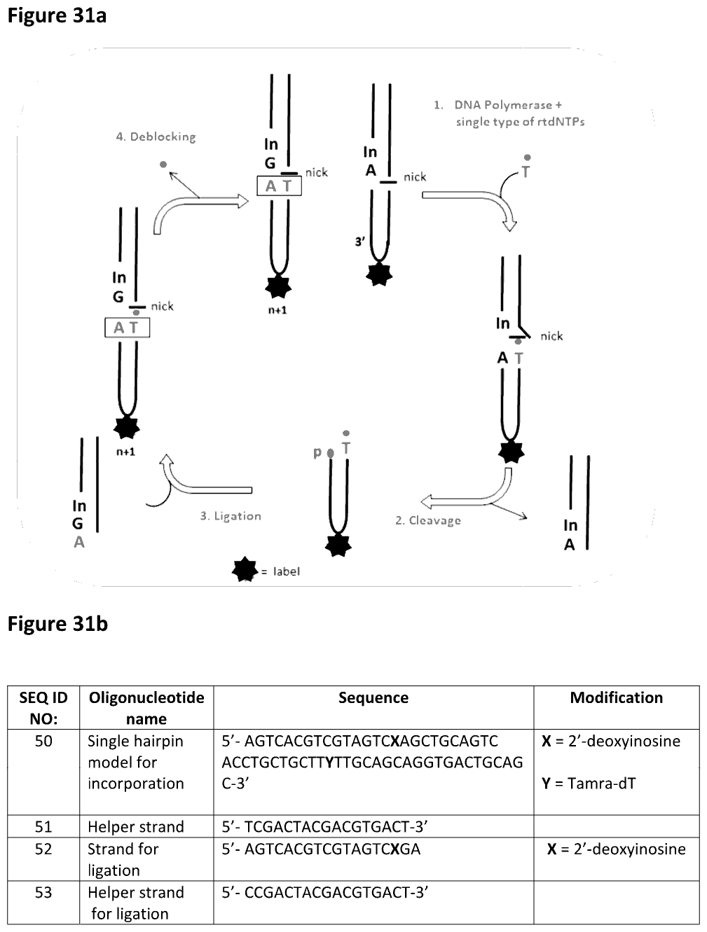

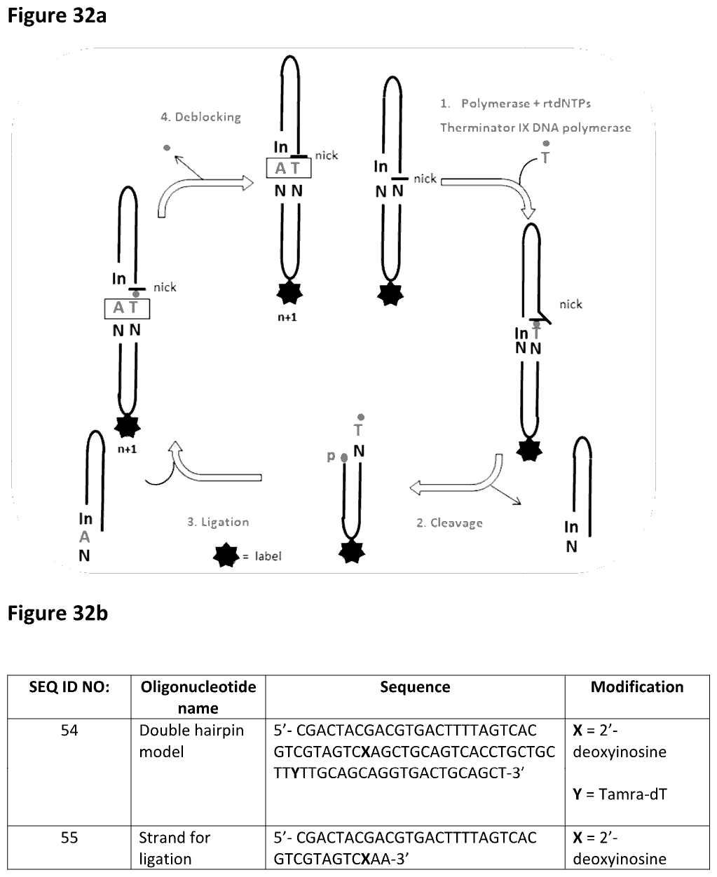

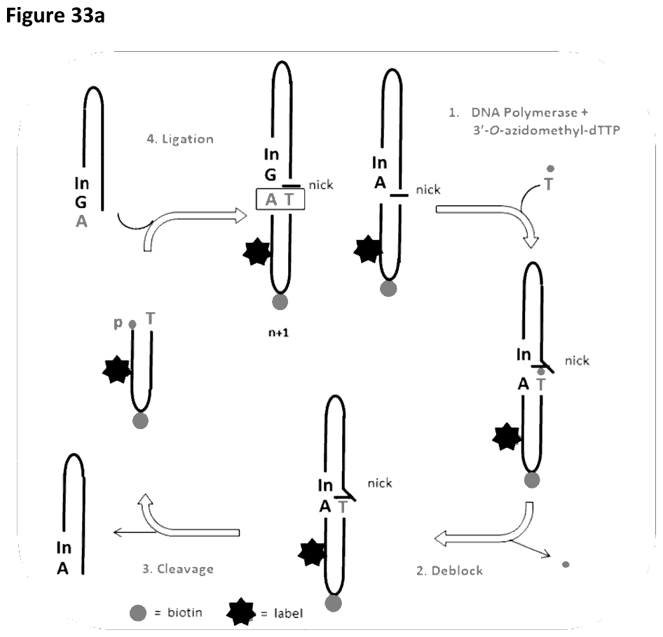

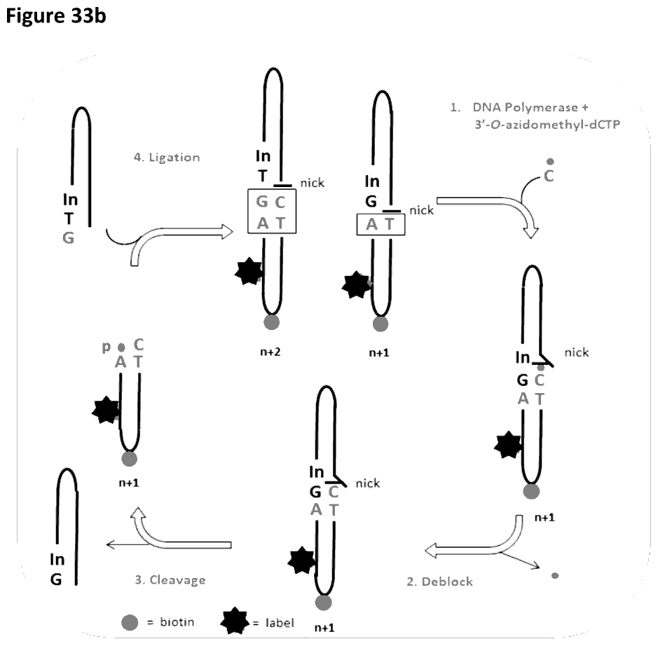

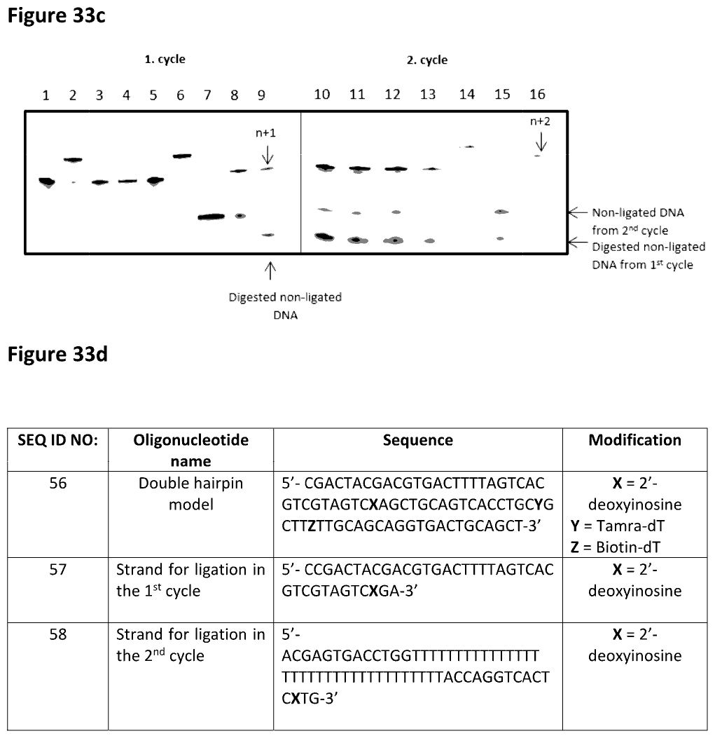

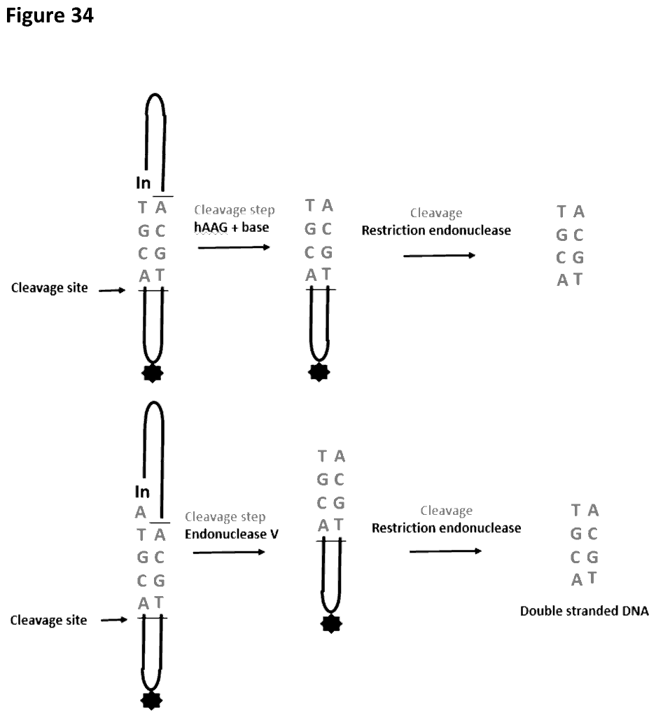

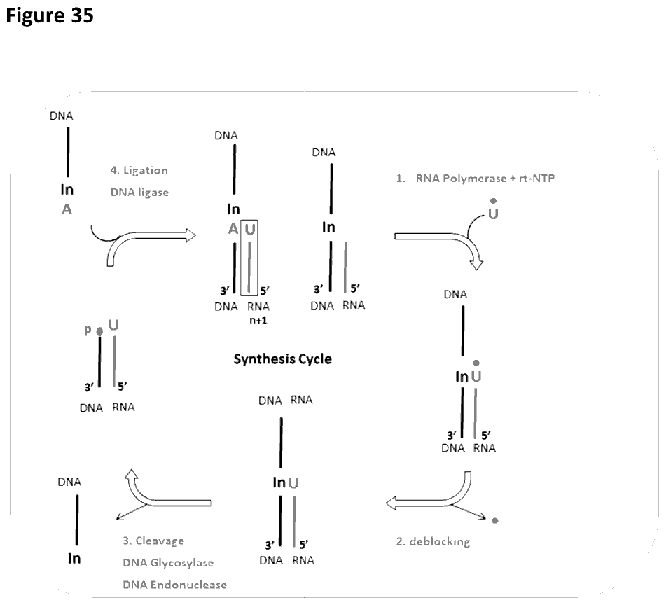

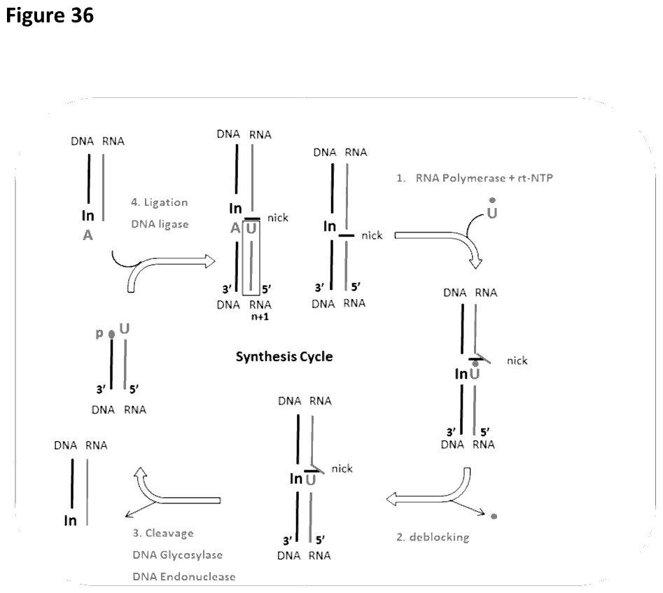

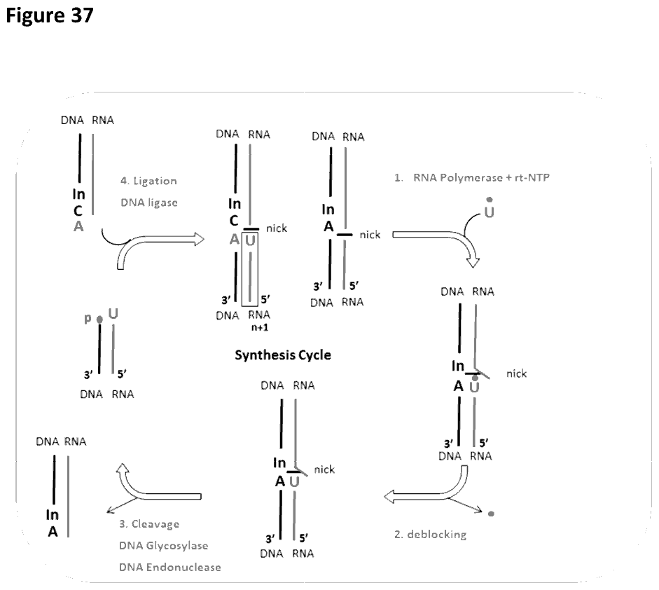

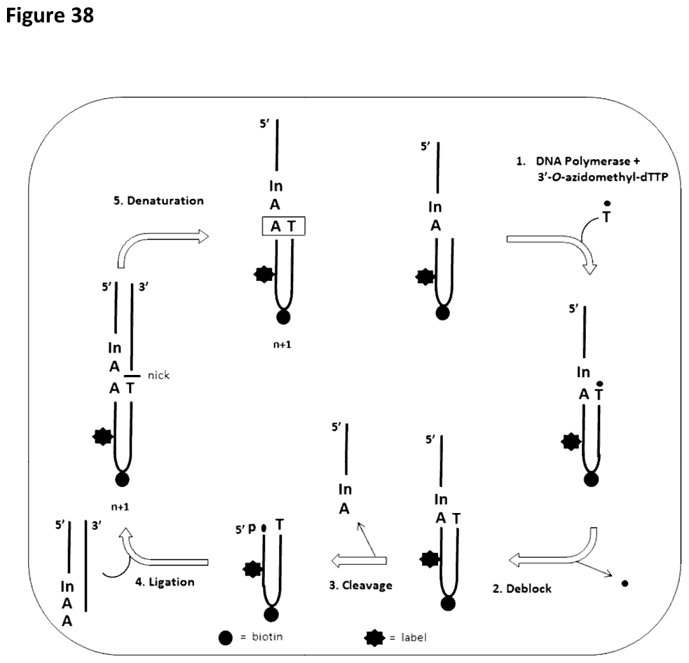

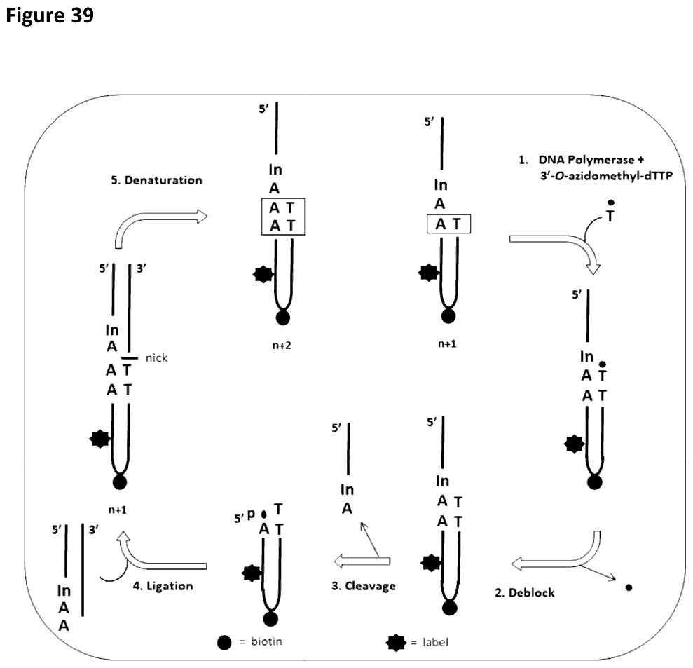

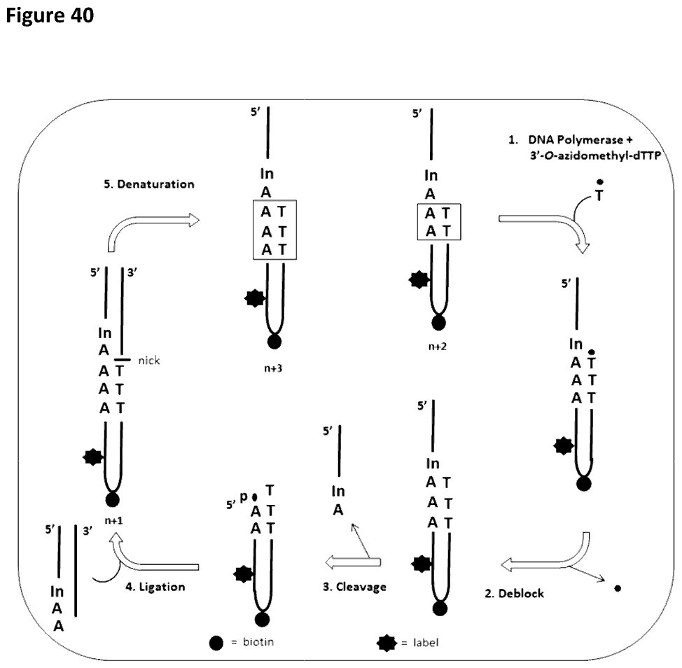

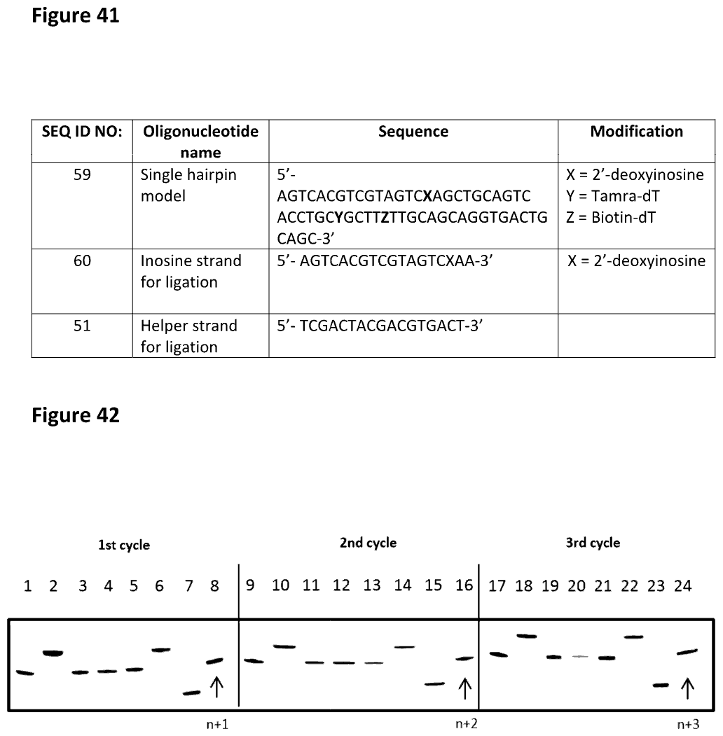

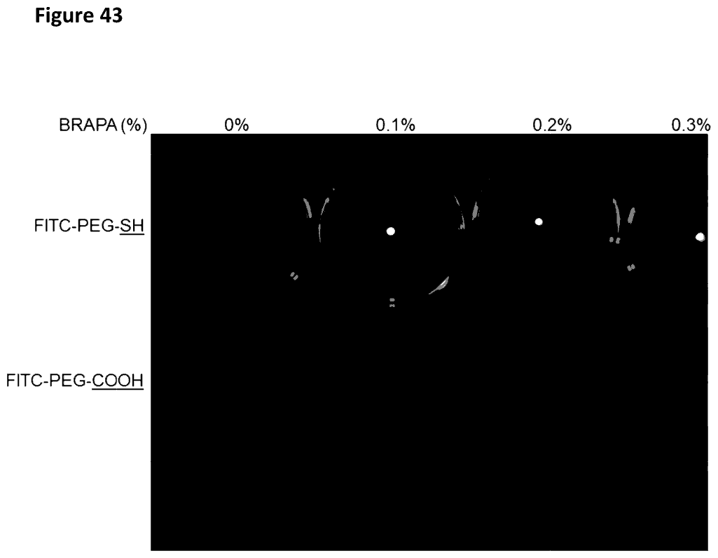

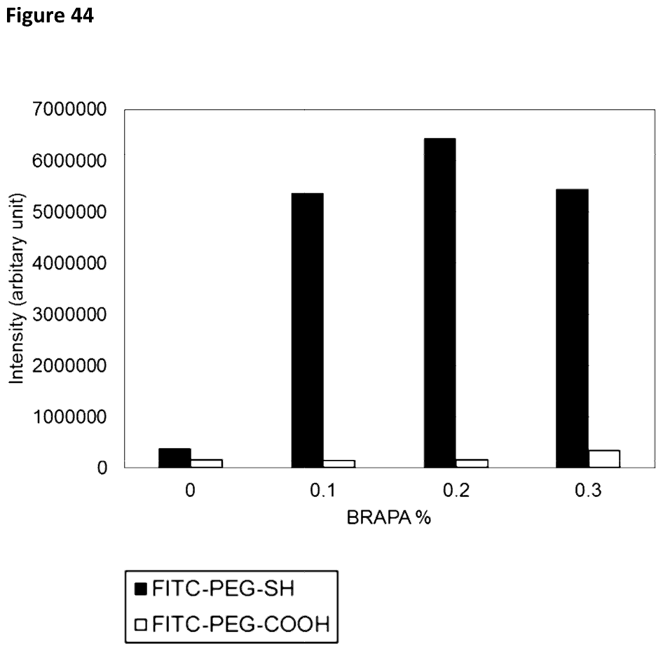

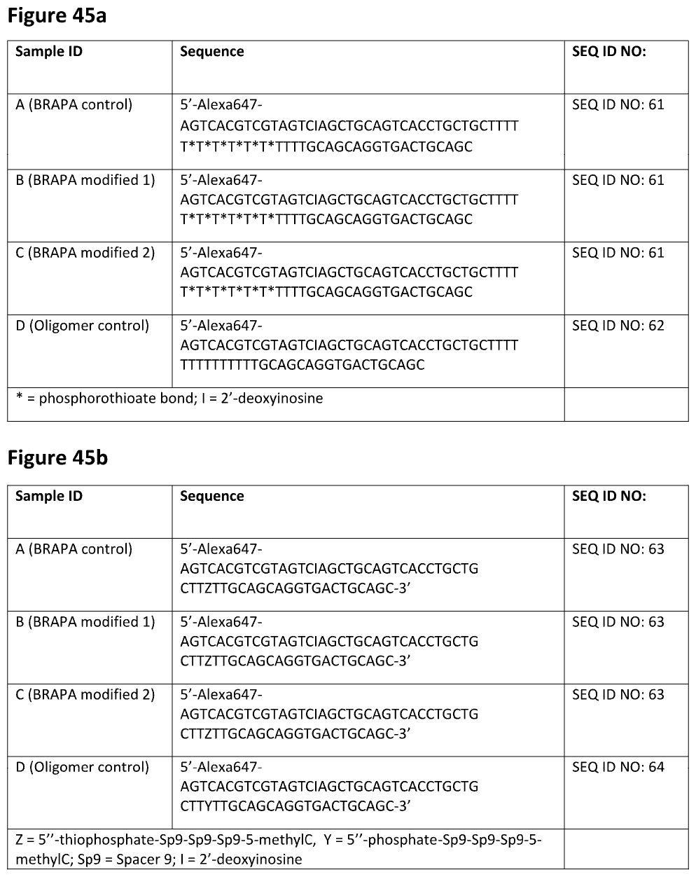

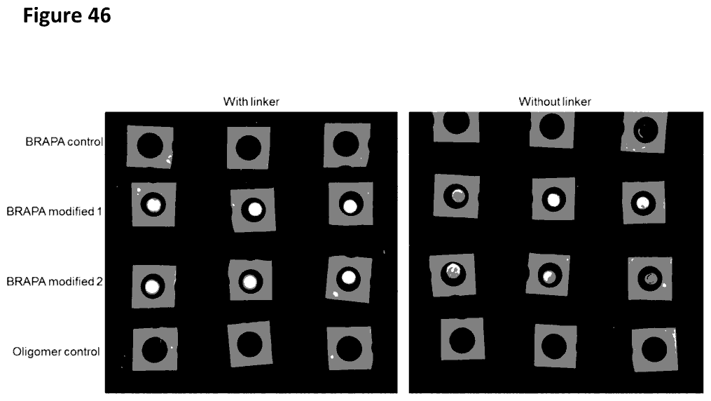

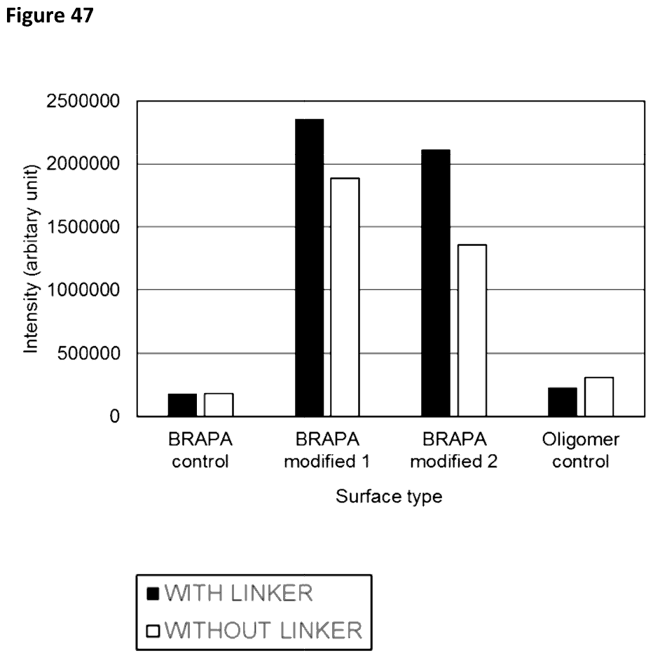

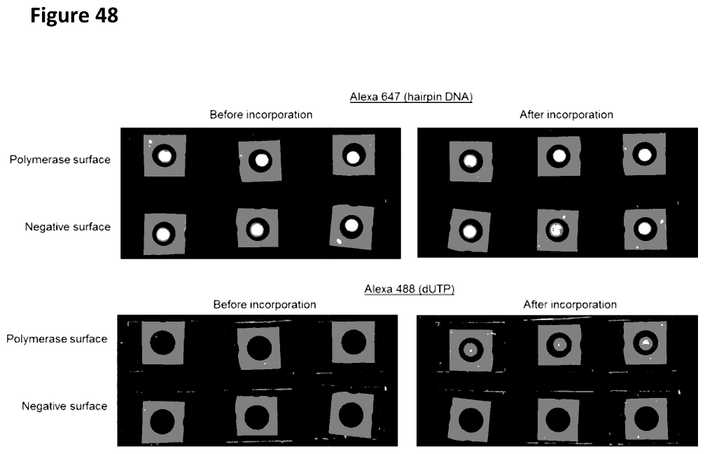

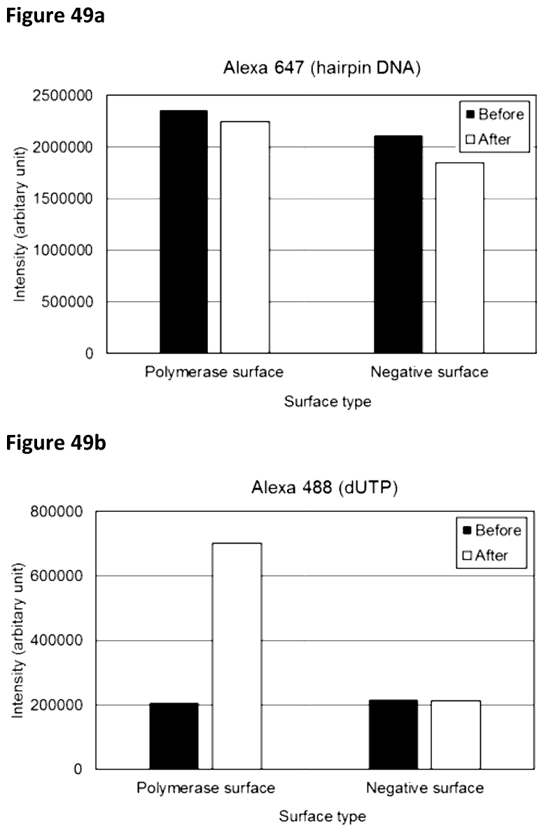

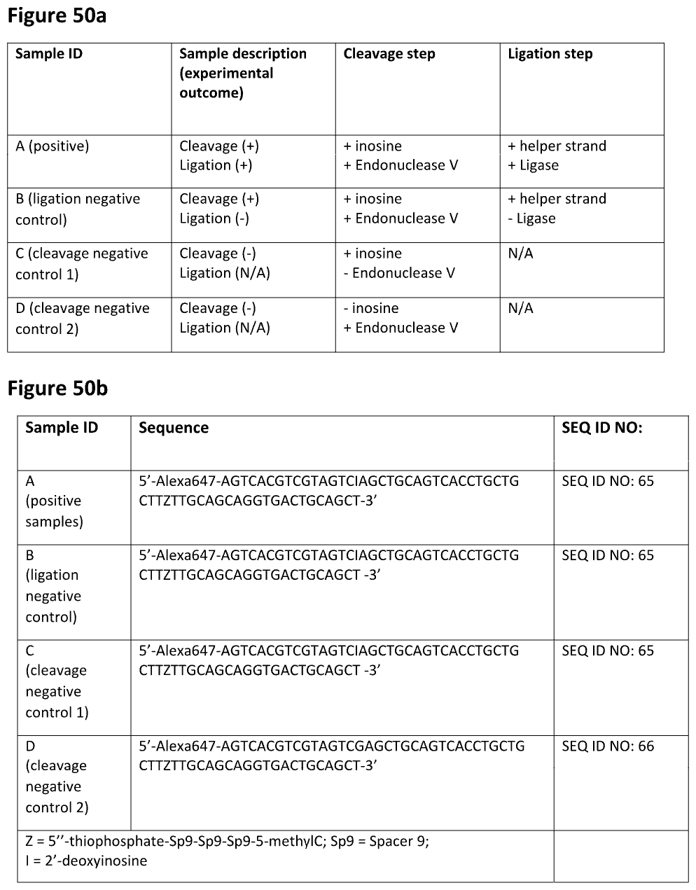

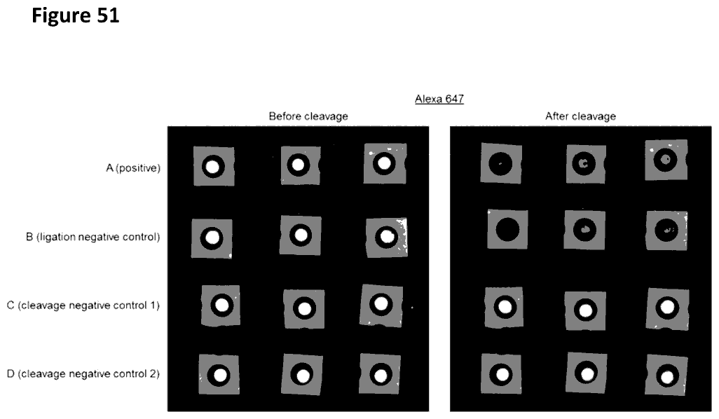

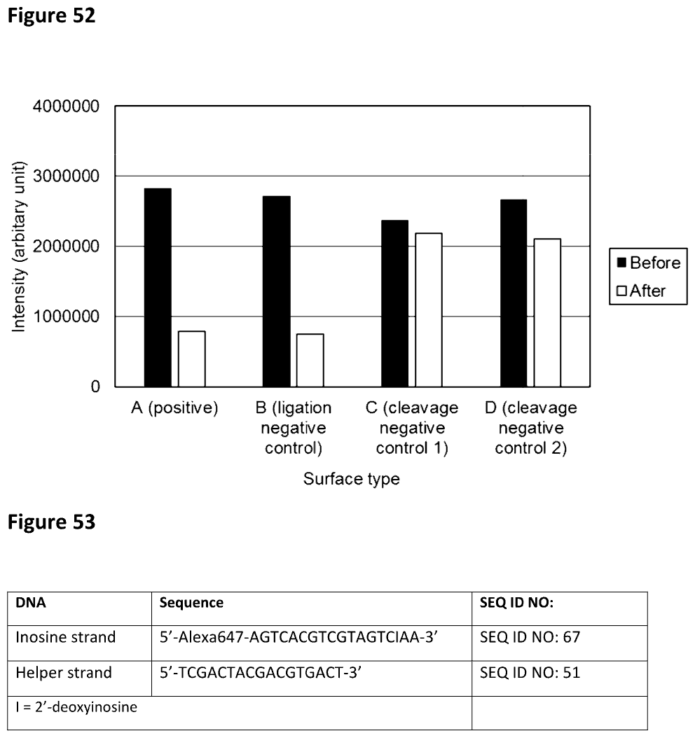

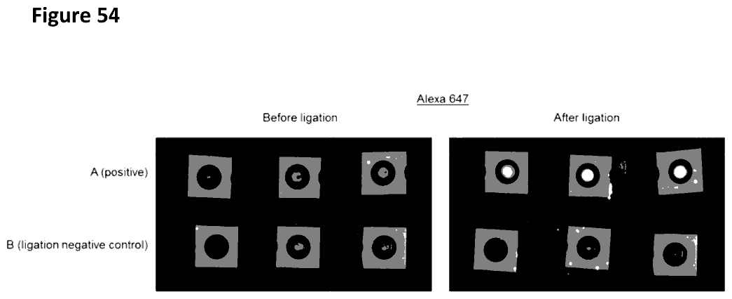

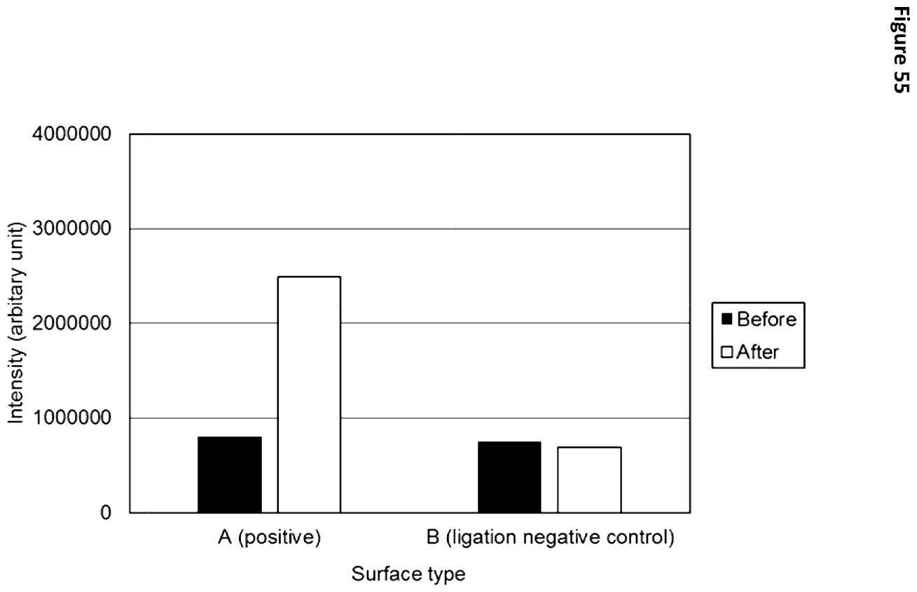

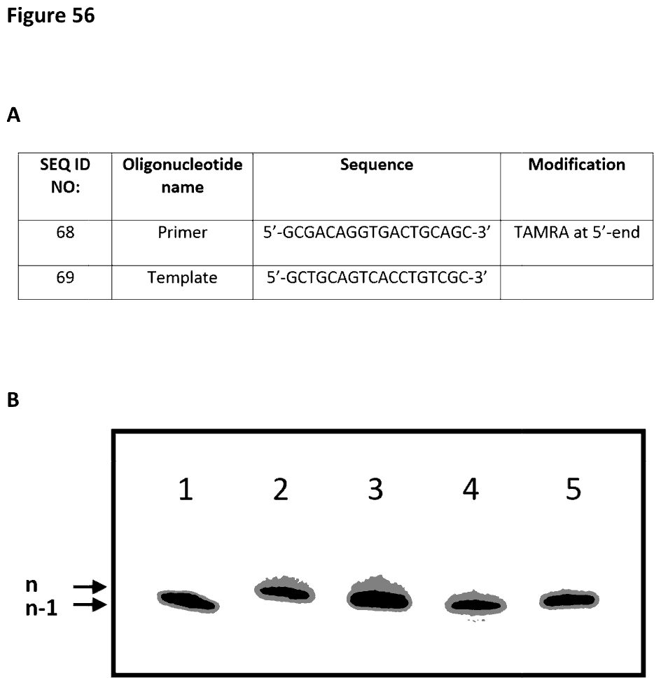

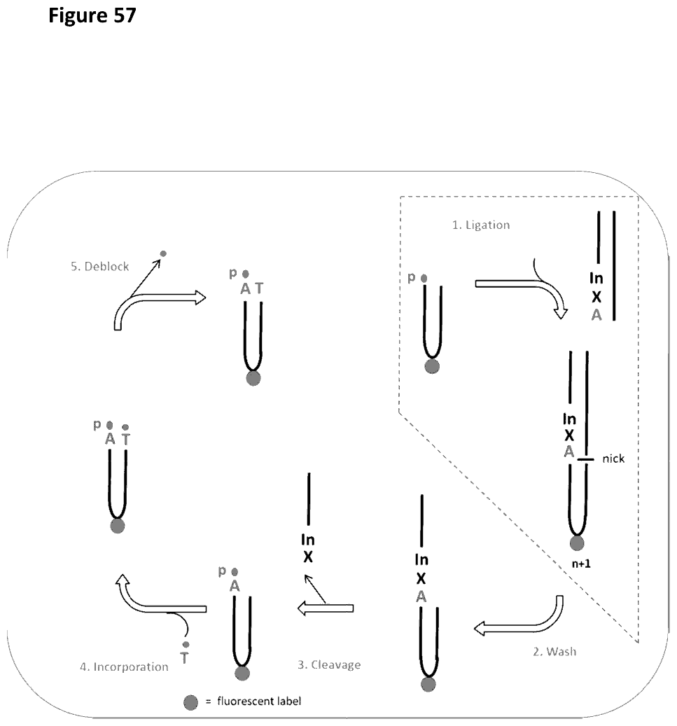

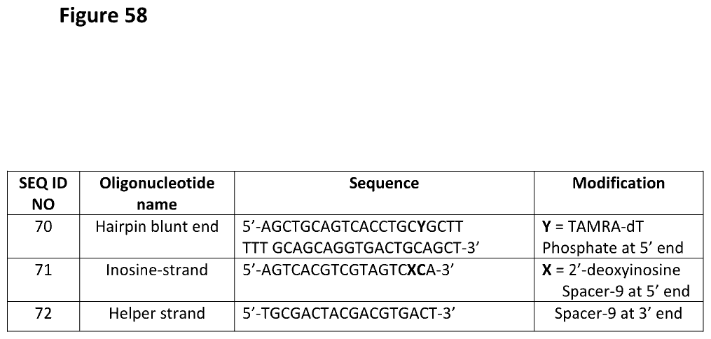

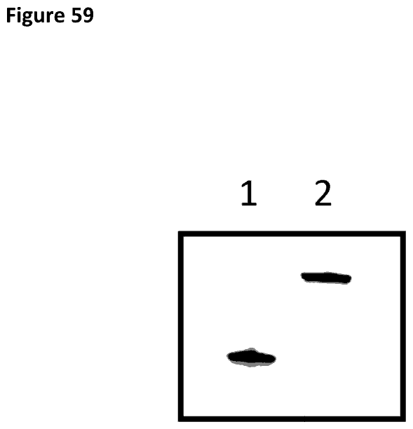

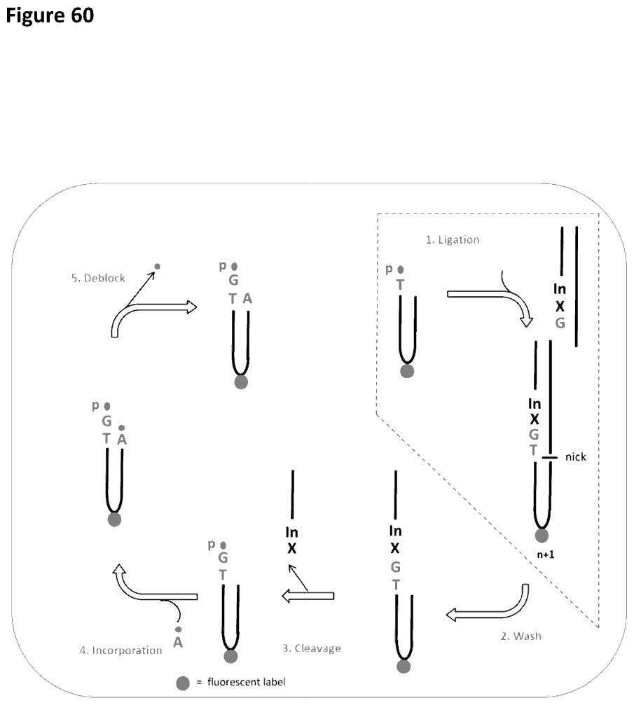

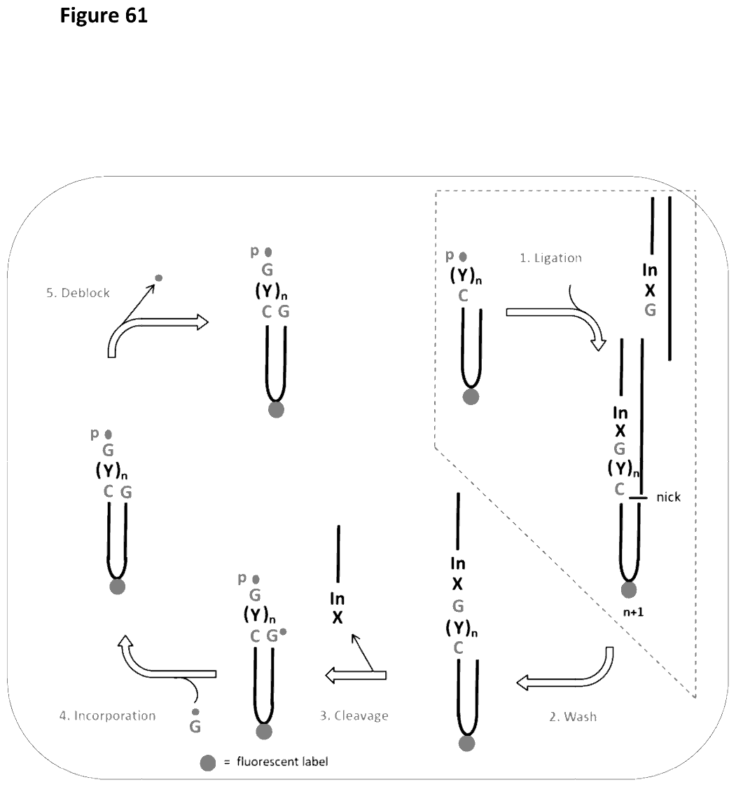

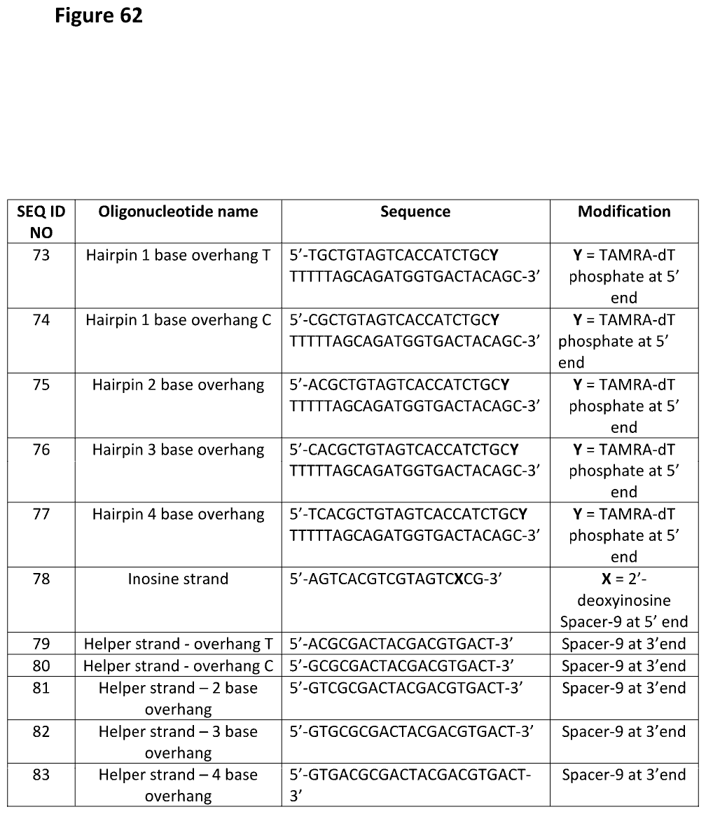

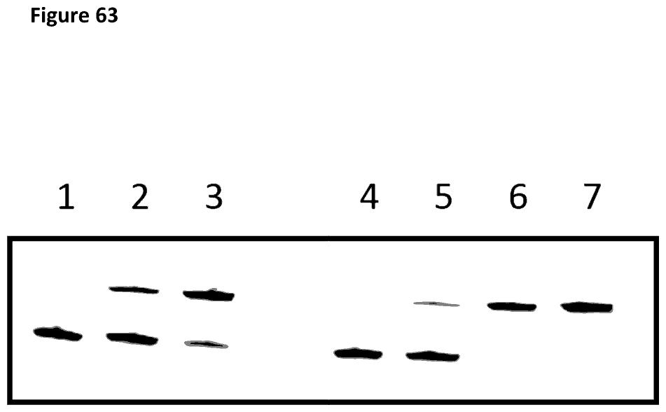

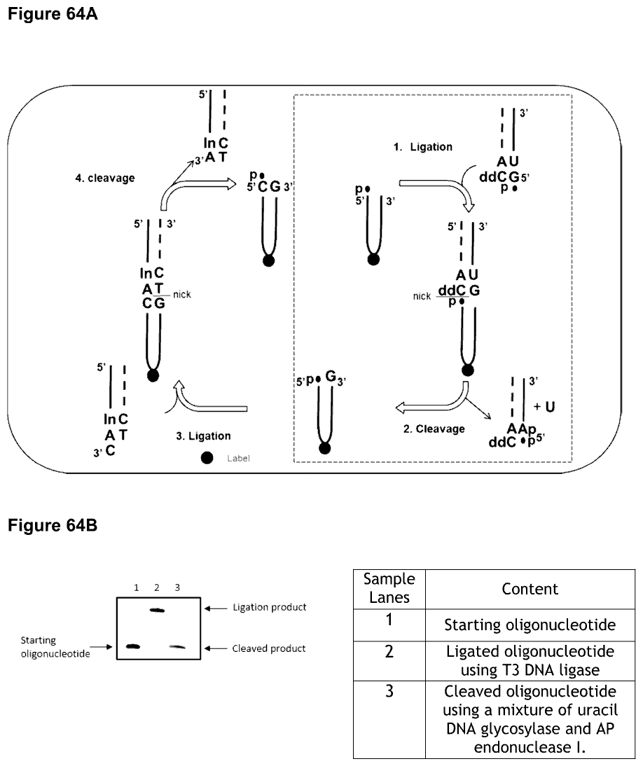

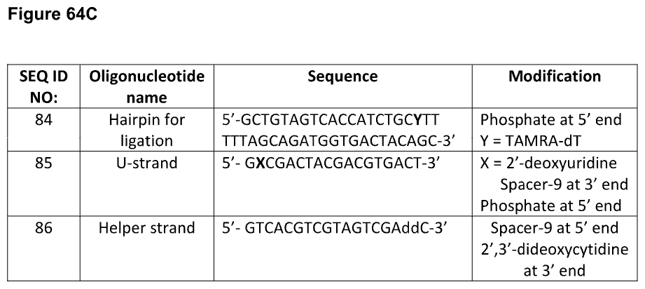

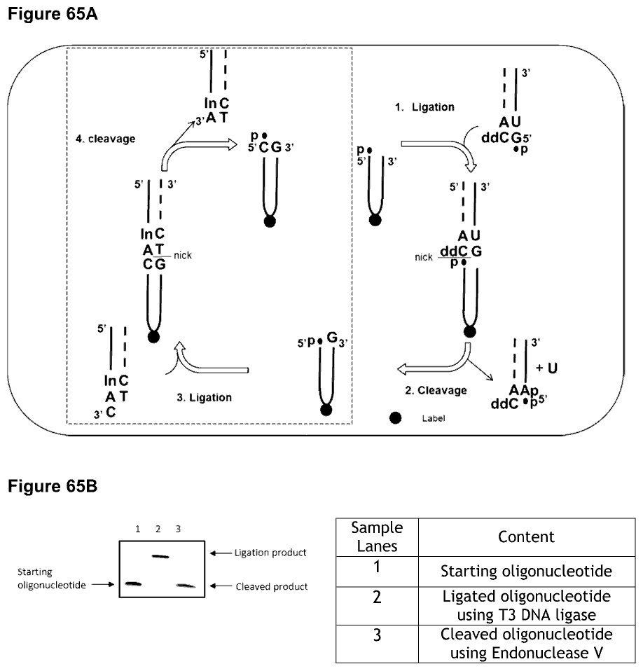

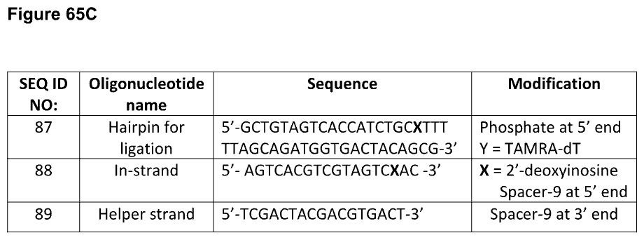

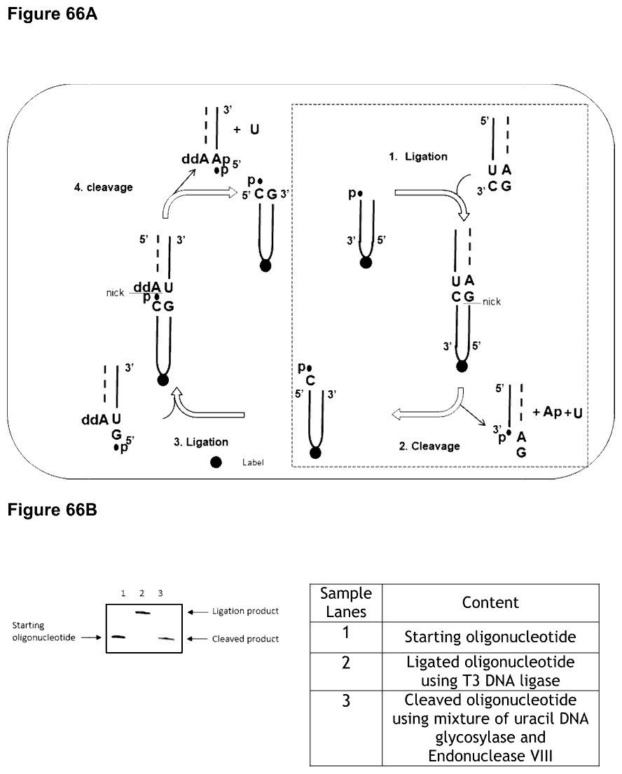

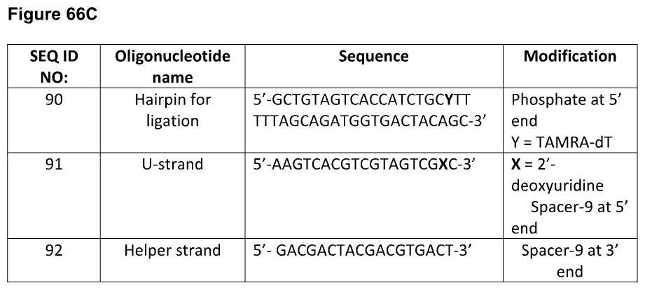

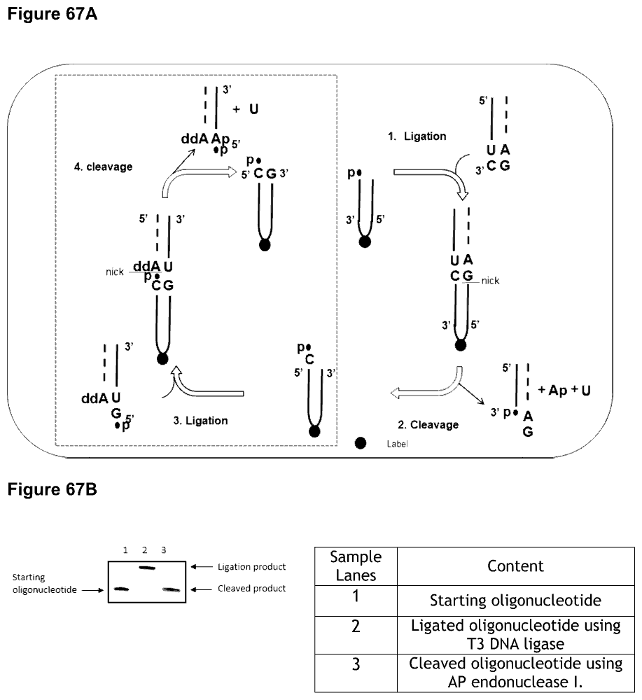

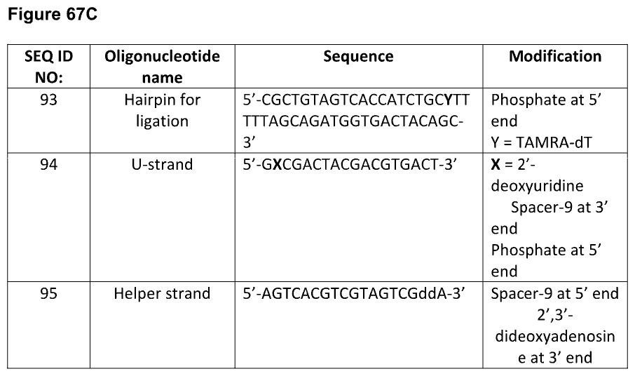

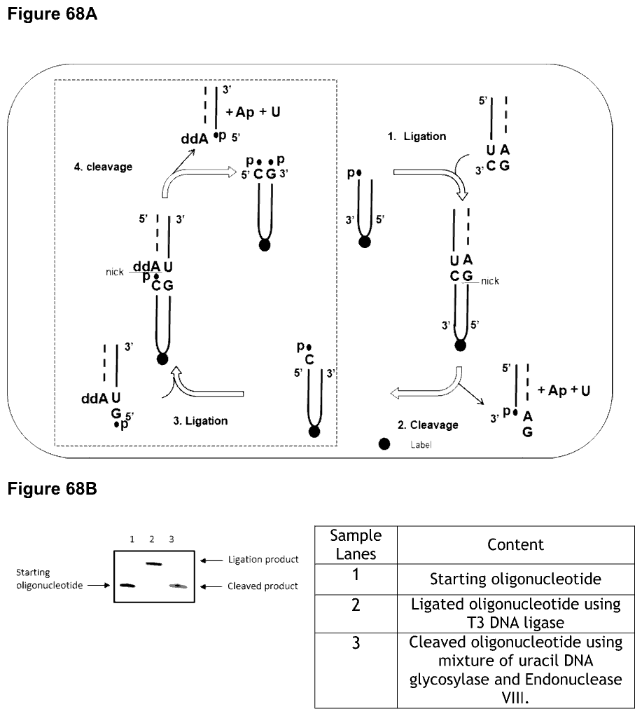

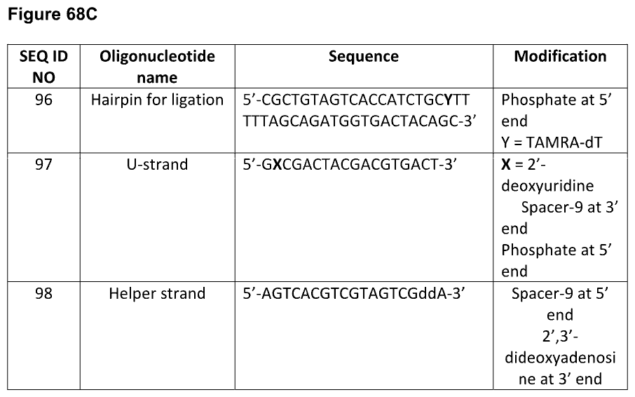

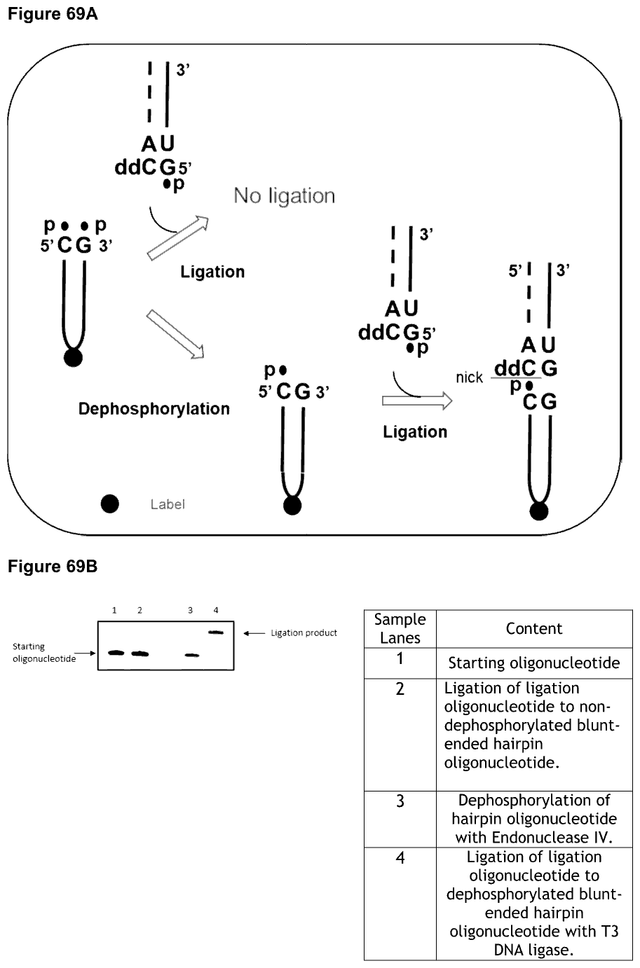

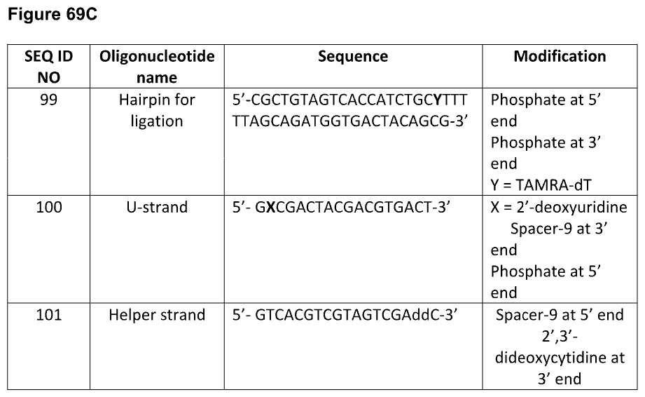

Results relating to incorporation of 3′-O-modified-dNTPs by Therminator X DNA polymerase using an incorporation step according to methods of the invention, e.g. synthesis method versions of the invention 1, 2, 3 and 4 and variants thereof (FIGS. 1 to 10 and Example 13).

FIG. 56a provides the nucleic acid sequences of primer strand (primer strand portion of synthesis strand; SEQ ID NO: 68) and template strand (support strand; SEQ ID NO: 69).

FIG. 56b depicts a gel showing the results of incorporation of 3′-O-modified-dNTPs by Therminator X DNA polymerase in presence of Mn2+ ions at 37° C.

Lane 1: Starting oligonucleotide.

Lane 2: Incorporation of 3′-O-azidomethyl-dTTP (>99% efficiency)

Lane 3: Incorporation of 3′-O-azidomethyl-dATP (>99% efficiency).

Lane 4: Incorporation of 3′-O-azidomethyl-dCTP (>90% efficiency).

Lane 5: Incorporation of 3′-O-azidomethyl-dGTP (>99% efficiency).

Upon addition, the newly added 3′-O-modified-dNTP occupies position n in the primer strand portion. The next nucleotide position in the primer strand portion is designated n−1.

FIG. 57.

The Figure shows a scheme depicting a DNA synthesis reaction cycle as described in Example 14.

FIG. 58.

The Figure shows oligonucleotides used in the experiments described in Example 14.

FIG. 59.

The Figure shows a photograph of a gel demonstrating the results of the ligation of a polynucleotide ligation molecule comprising 2-deoxyinosine, used as a universal nucleotide, to a hairpin scaffold polynucleotide as described in Example 14. Lane 1 shows the starting hairpin scaffold polynucleotide and lane 2 shows the hairpin scaffold polynucleotide ligated to the polynucleotide ligation molecule.

FIGS. 60 and 61.

The Figures show schemes depicting DNA synthesis reaction cycles as described in Example 15.

FIG. 62.

The Figure shows oligonucleotides used in the experiments described in Example 15.

FIG. 63.

The Figure shows a photograph of a gel demonstrating the results of the ligation of polynucleotide ligation molecules comprising 2-deoxyinosine, used as a universal nucleotide, to hairpin scaffold polynucleotides as described in Example 15. The lanes of the gel are as follows:
Lane 1: Starting hairpin scaffold polynucleotide.
Lane 2: Hairpin scaffold polynucleotide ligated to polynucleotide ligation molecule (1 base T overhang).
Lane 3: Hairpin scaffold polynucleotide ligated to polynucleotide ligation molecule (1 base C overhang).
Lane 4: Starting hairpin scaffold polynucleotide.
Lane 5: Hairpin scaffold polynucleotide ligated to polynucleotide ligation molecule (2 bases overhang).
Lane 6: Hairpin scaffold polynucleotide ligated to polynucleotide ligation molecule (3 bases overhang).
Lane 7: Hairpin scaffold polynucleotide ligated to polynucleotide ligation molecule (4 bases overhang).

FIG. 64

(a) The Figure shows the insertion of a single guanosine nucleotide to the 3′ end of a hairpin polynucleotide by blunt end ligation of a polynucleotide ligation molecule comprising uridine in the presence of a helper strand, followed by site specific cleavage of the polynucleotide at the position of uridine (Example 16).

(b) The Figure shows a photograph of a gel demonstrating the results of the ligation of a polynucleotide ligation molecule comprising 2-deoxyuridine, used as a universal nucleotide, to the 3′ end of a blunt ended hairpin followed by cleavage of the uridine containing ligated polynucleotide as shown in Example 16.
Lane 1: shows the starting blunt ended hairpin polynucleotide.
Lane 2: shows the hairpin scaffold polynucleotide ligated to the polynucleotide ligation molecule using T3 DNA ligase.
Lane 3: shows the cleaved polynucleotide using a mixture of uracil DNA glycosylase and AP endonuclease I.

(c) The Figure shows oligonucleotides used in the experiments detailed in Example 16.

FIG. 65

(a) The Figure shows the insertion of a single cytidine nucleotide to the synthesis strand by ligation to the 5′ end of a hairpin polynucleotide with a 3′ single base overhang in the presence of a helper strand, followed by site-specific cleavage of the polynucleotide at the 2^{nd} phosphodiester bond 3′ to the position of inosine (Example 17).

(b) The Figure shows a photograph of a gel demonstrating the results of the ligation of a polynucleotide ligation molecule comprising 2-deoxyinosine, used as a universal nucleotide, to the 5′ end of an overhung hairpin, followed by cleavage of the inosine containing ligated polynucleotide as shown in Example 17.
Lane 1: shows the starting 3′ single base overhung hairpin polynucleotide.
Lane 2: shows the hairpin polynucleotide ligated to the polynucleotide ligation molecule using T3 DNA ligase.
Lane 3: shows the cleaved polynucleotide using Endonuclease V.

(c) The Figure shows oligonucleotides used in the experiments detailed in Example 17.

FIG. 66

(a) The Figure shows the insertion of a single cytidine nucleotide to the synthesis strand by ligation to the 5′ end of blunt-ended hairpin polynucleotide in the presence of a helper strand, followed by site-specific cleavage of the polynucleotide at the 1^{st} bond 3′ to the position of uridine (Example 18).

(b) The Figure shows a photograph of a gel demonstrating the results of the ligation of a polynucleotide ligation molecule comprising 2-deoxyuridine, used as a universal nucleotide, to the 5′ end of a blunt ended hairpin, followed by cleavage of the uracil containing ligated polynucleotide as shown in Example 18.
Lane 1: shows the starting blunt-ended hairpin polynucleotide.
Lane 2: shows the hairpin polynucleotide ligated to the polynucleotide ligation molecule using T3 DNA ligase.
Lane 3: shows the cleaved polynucleotide using a mixture of uracil DNA glycosylase and Endonuclease VIII.

(c) The Figure shows oligonucleotides used in the experiments detailed in Example 18.

FIG. 67

(a) The Figure shows the insertion of a single guanidine nucleotide to the synthesis strand by ligation to the 3′ end of a hairpin polynucleotide with a 5′ single base overhang in the presence of a helper strand, followed by site-specific cleavage of the polynucleotide at the 1^{st} phosphodiester bond 5′ to the position of uridine (Example 19).

(b) The Figure shows a photograph of a gel demonstrating the results of the ligation of a polynucleotide ligation molecule comprising 2-deoxyuridine, used as a universal nucleotide, to the 3′ end of an overhung hairpin, followed by cleavage of the uracil containing ligated polynucleotide as shown in Example 19.
Lane 1: shows the starting 5′ single base overhung hairpin polynucleotide.
Lane 2: shows the ligated hairpin polynucleotide ligated to the polynucleotide ligation molecule using T3 DNA ligase.
Lane 3: shows the cleaved polynucleotide using AP endonuclease I.

(c) The Figure shows oligonucleotides used in the experiments detailed in Example 19.

FIG. 68

(a) The Figure shows a scheme depicting the insertion of a single guanidine nucleotide to the synthesis strand by ligation to the 3′ end of a hairpin polynucleotide with a 5′ single base overhang in the presence of a helper strand, followed by site-specific cleavage of the polynucleotide at both 1^{st} phosphodiester bonds 5′ and 3′ to the position of uridine, leaving phosphate attached to the 3′-end of the hairpin polynucleotide (Example 20).

(b) The Figure shows a photograph of a gel demonstrating the results of the ligation of a polynucleotide ligation molecule comprising 2-deoxyuridine, used as a universal nucleotide, to the 3′ end of an overhung hairpin, followed by cleavage of the uracil containing ligated polynucleotide as shown in Example 20.
Lane 1: shows the starting 3′ single base overhung hairpin polynucleotide.
Lane 2: shows the ligated hairpin polynucleotide ligated to the polynucleotide ligation molecule using T3 DNA ligase.
Lane 3: show the cleaved polynucleotide using a mixture of uracil DNA glycosylase and Endonuclease VIII.

(c) The Figure shows oligonucleotides used in the experiments detailed in Example 20.

FIG. 69

(a) The Figure shows the removal of a 3′-phosphate from a blunt-ended hairpin polynucleotide by Endonuclease IV followed by ligation of a polynucleotide ligation molecule to the 3′ end of a blunt-ended hairpin polynucleotide. Ligation of a polynucleotide ligation molecule to a 3′-phosphorylated blunt-ended polynucleotide was performed as negative control (Example 21).

(b) The Figure shows a photograph of a gel demonstrating the results of the ligation of a polynucleotide ligation molecule to a non-dephosphorylated and a dephosphorylated blunt-ended hairpin polynucleotide.
Lane 1: shows the starting blunt ended hairpin polynucleotide.
Lane 2: shows the ligation of the polynucleotide ligation molecule to a non-dephosphorylated (i.e. phosphorylated) blunt-ended hairpin polynucleotide
Lane 3: shows the desphosphorylated hairpin oligonucleotide using Endonuclease IV.
Lane 4: shows the ligation of the polynucleotide ligation molecule to a dephosphorylated blunt-ended hairpin polynucleotide using T3 DNA ligase.

(c) The Figure shows oligonucleotides used in the experiments detailed in Example 21.

### INTERPRETATION OF FIGURES

The structures depicted in FIGS. 16, 17a, 18a, 19a, 20a, 21a, 22a, 23a, 24a, 25a, 26a, 27a, 28a, 29a, 30a, 31a, 32a, 33a, 33b, 34, 35, 36, 37, 38, 39, and 40 are to be interpreted consistently with those depicted in FIGS. 11, 12, 13, 14 and 15. Thus in these Figures, each left hand strand of a double-stranded scaffold polynucleotide molecule relates to the support strand (corresponding to strand “a” in FIGS. 11 to 15); each right hand strand of a double-stranded scaffold polynucleotide molecule relates to the synthesis strand (corresponding to strand “b” in FIGS. 11 to 15); all scaffold polynucleotide molecules comprise a lower synthesis strand which corresponds to a strand comprising a primer strand portion (corresponding to the solid and dotted line of strand “b” in FIGS. 6 to 10); certain scaffold polynucleotide molecules (e.g. in FIGS. 20a and 28a) are shown, prior to incorporation of the new nucleotide, with an upper synthesis strand which corresponds to a strand comprising a helper strand portion (corresponding to the dashed line of strand “b” in FIGS. 11 to 15); certain scaffold polynucleotide molecules (e.g. in FIGS. 17a, 18a and 19a) are shown with no helper strand portion (corresponding to an absence of the dashed line of strand “b” in FIGS. 11 to 15); and certain scaffold polynucleotide molecules (e.g. in FIGS. 38, 39 and 40) are shown, after the ligation step, with an upper synthesis strand which corresponds to a strand comprising a helper strand portion (corresponding to the dashed line of strand “b” in FIGS. 11 to 15) and wherein the helper strand portion is removed prior to incorporation of the new nucleotide in the next synthesis cycle.

In addition, in these Figures, where relevant, each new nucleotide is shown to be incorporated together with a reversible terminator group, labelled rtNTP and depicted as a small circular structure (corresponding to the small triangular structure in FIGS. 11 to 15) and terminal phosphate groups are labelled “p” and depicted as a small elliptical structure.

FIGS. 16c, 16d, 16g, 16h, 27a, 28a, 29a, 30a, 32a, 33a, 33b, and 34 show scaffold polynucleotide molecules wherein strands comprising a helper strand portion and support strands are connected by a hairpin loop. FIGS. 16b, 27a, 28a, 29a, 30a, 31a, 32a, 33a, 33b, 34, 38, 39, and 40 show scaffold polynucleotide molecules wherein strands comprising a primer strand portion and support strands are connected by a hairpin loop.

Figures such as FIGS. 32a and 33a show scaffold polynucleotide molecules wherein the strand comprising a helper strand portion (upper right strand) and the support strand (upper left strand) is connected by a hairpin loop and, in the same molecule, the strand comprising the primer strand portion (lower right strand) and the support strand (lower left strand) are connected by a hairpin loop. In relation to these Figures and corresponding methods, a complete explanation of the structures referred to as a scaffold polynucleotide molecule, a support strand, a synthesis strand, a primer strand portion, a helper strand portion and a polynucleotide ligation molecule, and methods relating to the incorporation into scaffold polynucleotide molecules or nucleotides comprising reversible terminator groups are provided in international patent application publication WO2018/134616.

### DETAILED DESCRIPTION OF THE INVENTION

The present invention provides methods for the de novo synthesis of polynucleotide molecules according to a predefined nucleotide sequence. Synthesised polynucleotides are preferably DNA and are preferably double-stranded polynucleotide molecules. The invention provides advantages compared with existing synthesis methods. For example, all reaction steps may be performed in aqueous conditions at mild pH, extensive protection and deprotection procedures are not required. Furthermore, synthesis is not dependent upon the copying of a pre-existing template strand comprising the predefined nucleotide sequence.

The present inventors have determined that the use of a “universal nucleotide”, as defined herein, allows for the creation of a polynucleotide cleavage site within a synthesised region which facilitates cleavage and repeat cycles of synthesis. The invention provides versatile methods for synthesising polynucleotides, and for assembling large fragments comprising such synthesised polynucleotides.

Certain embodiments of the synthesis methods of the invention will be described in more general detail herein by reference to exemplary synthesis method versions of the invention and certain variants thereof (FIGS. 1 to 10 and descriptions of the same). It is to be understood that all exemplary methods, including the exemplary method versions of the invention and variants thereof, are not intended to be limiting on the invention. The invention provides an in vitro method of synthesising a double-stranded polynucleotide molecule having a predefined sequence, the method comprising performing cycles of synthesis wherein in each cycle a first polynucleotide strand is extended by the incorporation of a first nucleotide of the predefined sequence, and then the second polynucleotide strand which is hybridized to the first strand is extended by the incorporation of a second nucleotide of the predefined sequence. Preferably, the methods are for synthesising DNA. Specific methods described herein are provided as embodiments of the invention.

### Reaction Conditions

In one aspect the invention provides a method for synthesising a double-stranded polynucleotide having a predefined sequence.

Synthesis is carried out under conditions suitable for hybridization of nucleotides within double-stranded polynucleotides. Polynucleotides are typically contacted with reagents under conditions which permit the hybridization of nucleotides to complementary nucleotides. Conditions that permit hybridization are well-known in the art (for example, Sambrook et al., 2001, Molecular Cloning: a laboratory manual, 3rd edition, Cold Spring Harbour Laboratory Press; and Current Protocols in Molecular Biology, Greene Publishing and Wiley-Interscience, New York (1995)).

Cleavage of polynucleotides can be carried out under suitable conditions, for example using a polynucleotide cleaving enzyme (e.g., endonuclease) at a temperature that is compatible with the enzyme (e.g., 37° C.) in the presence of a suitable buffered solution. In one embodiment, the buffered solution can comprise 5 mM potassium acetate, 2 mM Tris-acetate, 1 mM magnesium acetate and 0.1 mM DTT.

Ligation of polynucleotides can be carried out under suitable conditions, for example using a ligase (e.g., T4 DNA ligase) at a temperature that is compatible with the enzyme (e.g., room temperature) in the presence of a suitable buffered solution. In one embodiment, the buffered solution can comprise 4.4 mM Tris-HCl, 7 mM MgCl₂, 0.7 mM dithiothreitol, 0.7 mM ATP, 5% polyethylene glycol (PEG6000).

### Anchor Polynucleotides and Scaffold Polynucleotides

Double-stranded polynucleotides having a predefined sequence are synthesised by methods of the invention by incorporation of pre-defined nucleotides into a pre-existing polynucleotide, referred to herein as a scaffold polynucleotide, which may be attached to or capable of being attached to a surface as described herein. As described in more detail herein a scaffold polynucleotide forms a support structure to accommodate the newly-synthesised polynucleotide and, as will be apparent from the description herein, does not comprise a pre-existing template strand which is copied as in conventional methods of synthesis. A scaffold polynucleotide may be referred to as an anchor polynucleotide if the scaffold polynucleotide is attached to a surface. Surface attachment chemistries for attaching a scaffold polynucleotide to a surface to form an anchor polynucleotide are described in more detail herein.

A scaffold polynucleotide comprises a first strand hybridized to a complementary second strand (e.g. see FIGS. 1 to 10). The first strand may be provided hybridized to the complementary second strand. Alternatively, the first strand and the second strand may be provided separately and then allowed to hybridise.

A scaffold polynucleotide may be provided with each of the first and second strands unconnected at adjacent ends. A scaffold polynucleotide may be provided with both first and second strands connected at adjacent ends, such as via a hairpin loop, at both ends of the scaffold polynucleotide. A scaffold polynucleotide may be provided with both first and second strands connected at adjacent ends, such as via a hairpin loop, at one end of the scaffold polynucleotide or any other suitable linker.

Scaffold polynucleotides with or without hairpins may be immobilized to a solid support or surface as described in more detail herein (see FIG. 12).

The terms “hairpin” or “hairpin loop” are commonly used in the current technical field. The term “hairpin loop” is also often referred to as a “stem loop”. Such terms refer to a region of secondary structure in a polynucleotide comprising a loop of unpaired nucleobases which form when one strand of a polynucleotide molecule hybridizes with another section of the same strand due to intramolecular base pairing. Thus hairpins can resemble U-shaped structures. Examples of such structures are shown in FIG. 12.

In the methods described herein a first extension step is performed by a first extension/ligation reaction thereby incorporating into the first strand of the scaffold polynucleotide a first nucleotide or first and second nucleotides of the predefined sequence by the action of a ligase enzyme. Thus the first nucleotide or first and second nucleotides of the predefined sequence are ligated to the terminal nucleotide of the first strand of the scaffold polynucleotide as described further herein. The first nucleotide of the predefined sequence or the first and second nucleotides of the predefined sequence are provided by a first polynucleotide ligation molecule which comprises a synthesis strand, a helper strand and a complementary ligation end. The first nucleotide of the predefined sequence or the first and second nucleotides of the predefined sequence are provided as the terminal nucleotide(s) of the synthesis strand of the complementary ligation end.

Following the first extension/ligation reaction a first cleavage step is performed, as described in more detail herein, to release the first polynucleotide ligation molecule from the scaffold polynucleotide and whereby the first nucleotide or first and second nucleotides of the predefined sequence of the first polynucleotide ligation molecule are retained attached to the first strand of the scaffold polynucleotide.

Following the first cleavage step a second extension step is performed in a second extension/ligation reaction thereby incorporating a first nucleotide or first and second nucleotides of the predefined sequence into the second strand of the scaffold polynucleotide by the action of a ligase enzyme. Thus the first nucleotide or first and second nucleotides of the predefined sequence to be incorporated into the second strand are ligated to the terminal nucleotide of the second strand of the scaffold polynucleotide as described further herein. The first nucleotide of the predefined sequence or the first and second nucleotides of the predefined sequence to be incorporated into the second strand are provided by a second polynucleotide ligation molecule which comprises a synthesis strand, a helper strand and a complementary ligation end. The first nucleotide of the predefined sequence or the first and second nucleotides of the predefined sequence to be incorporated into the second strand are provided as the terminal nucleotide(s) of the synthesis strand of the complementary ligation end.

Following the second extension/ligation reaction a second cleavage step is performed, as described in more detail herein, to release the second polynucleotide ligation molecule from the scaffold polynucleotide and whereby the first nucleotide or first and second nucleotides of the predefined sequence of the second polynucleotide ligation molecule are retained attached to the second strand of the scaffold polynucleotide.

In both the first and second polynucleotide ligation molecules the terminal nucleotide of the helper strand at the complementary ligation end is a non-ligatable nucleotide. If the non-ligatable nucleotide is provided at a 5′ end of the helper strand it is typically provided lacking a phosphate group. This prevents the terminal nucleotide of the helper strand ligating with the terminal nucleotide of the first or second strands of the scaffold polynucleotide and creates a single-strand break site between the helper strand and the first or second strands following ligation. Creation and maintenance of the single-strand break could be effected by other means. For example, the 5′terminal nucleotide of the helper strand may be provided with any suitable 5′ blocking group which prevents ligation with the first or second strands. If the non-ligatable nucleotide is provided at a 3′ end of the helper strand it is typically provided as a non-ligatable nucleotide comprising a non-ligatable 2′,3′-dideoxynucleotide or a 2′-deoxynucleotide, or comprising any other suitable 3′ non-ligatable nucleotide.

Further details of the general method schemes of exemplary methods are provided further herein.

### First and Second Strands of a Scaffold Polynucleotide

The first and second strands of a scaffold polynucleotide should be suitable to allow an enzyme having ligase activity to catalyse the ligation of a polynucleotide ligation molecule to the scaffold polynucleotide as described further herein. In addition, if further extension of either strand is desired, the first and second strands of a scaffold polynucleotide should be suitable to allow an enzyme, such as a polymerase enzyme or enzyme having terminal transferase activity, to initiate synthesis, i.e. catalyse the addition of a new nucleotide at the terminal end of the scaffold polynucleotide.

There are no special requirements for the parameters of length, sequence and structure of the first or second strand of the scaffold polynucleotide, provided that the first and second strands of the scaffold polynucleotide are suitable to facilitate ligation as described further herein, and to prime new polynucleotide synthesis as described further herein if desired.

The first and second strands may comprise nucleotides, nucleotide analogues/derivatives and/or non-nucleotides.

The skilled person is readily able to construct a scaffold polynucleotide comprising first and second strands which will be suitable to facilitate ligation as described further herein and which are capable of priming new polynucleotide synthesis as described further herein if desired. At the end of the scaffold polynucleotide which is to be extended mismatches with the support strand should be avoided, GC- and AT-rich regions should be avoided, and in addition regions of secondary structure such as hairpins or bulges which might interfere with ligation and/or other extension should be avoided.

Prior to the initiation of synthesis the length of the first and second strands of the scaffold polynucleotide can be chosen by the skilled person depending on preference and the ligase enzyme to be used.

The first strand must be hybridized to the corresponding region of the second strand. It is not essential that the entirety of the first strand is hybridized to the corresponding region of the second strand, provided that first and second strands are suitable for ligation as described herein, or capable of priming new polynucleotide synthesis as described further herein if desired. Thus, mismatches between the first strand and the corresponding region of the second strand can be tolerated to a degree. Preferably, the region of sequence of the first and second strands at the end of the scaffold polynucleotide to be extended should comprise nucleobases which are complementary to corresponding nucleobases in the support strand.

The first strand may be connected to the corresponding region of the second strand at the end of the scaffold polynucleotide which is not to be extended, e.g. via a hairpin.

### Nucleotides and Universal Nucleotides

Nucleotides which can be incorporated into synthetic polynucleotides by any of the methods described herein may be nucleotides, nucleotide analogues and modified nucleotides.

Nucleotides may comprise natural nucleobases or non-natural nucleobases. Nucleotides may contain a natural nucleobase, a sugar and a phosphate group. Natural nucleobases comprise adenosine (A), thymine (T), uracil (U), guanine (G) and cytosine (C). One of the components of the nucleotide may be further modified.

Nucleotide analogues are nucleotides that are modified structurally either in the base, sugar or phosphate or combination therein and that are still acceptable to a polymerase enzyme as a substrate for incorporation into an oligonucleotide strand.

A non-natural nucleobase may be one which will bond, e.g. hydrogen bond, to some degree to all of the nucleobases in the target polynucleotide. A non-natural nucleobase is preferably one which will bond, e.g. hydrogen bond, to some degree to nucleotides comprising the nucleosides adenosine (A), thymine (T), uracil (U), guanine (G) and cytosine (C).

A non-natural nucleotide may be a peptide nucleic acid (PNA), a locked nucleic acid (LNA) and an unlocked nucleic acid (UNA), a bridged nucleic acid (BNA) or a morpholino, a phosphorothioate or a methylphosphonate.

A non-natural nucleotide may comprise a modified sugar and/or a modified nucleobase. Modified sugars include but are not limited to 2′-O-methylribose sugar. Modified nucleobases include but are not limited to methylated nucleobases. Methylation of nucleobases is a recognised form of epigenetic modification which has the capability of altering the expression of genes and other elements such as microRNAs. Methylation of nucleobases occurs at discrete loci which are predominately dinucleotide consisting of a CpG motif, but may also occur at CHH motifs (where H is A, C, or T). Typically, during methylation a methyl group is added to the fifth carbon of cytosine bases to create methylcytosine. Thus modified nucleobases include but are not limited to 5-methylcytosine.

Nucleotides of the predefined sequence may be incorporated opposite partner nucleotides to form a nucleotide pair. A partner nucleotide may be a complementary nucleotide. A complementary nucleotide is a nucleotide which is capable of bonding, e.g. hydrogen bonding, to some degree to the nucleotides of the predefined sequence.

Typically, a nucleotide of the predefined sequence is incorporated into a polynucleotide opposite a naturally complementary partner nucleobase. Thus adenosine may be incorporated opposite thymine and vice versa. Guanine may be incorporated opposite cytosine and vice versa. Alternatively, a nucleotide of the predefined sequence may be incorporated opposite a partner nucleobase to which it will bond, e.g. hydrogen bond, to some degree.

Alternatively a partner nucleotide may be a non-complementary nucleotide. A non-complementary nucleotide is a nucleotide which is not capable of bonding, e.g. hydrogen bonding, to the nucleotide of the predefined sequence. Thus a nucleotide of the predefined sequence may be incorporated opposite a partner nucleotide to form a mismatch, provided that the synthesised polynucleotide overall is double-stranded and wherein the first strand is attached to the second strand by hybridization.

The term “opposite” is to be understood as relating to the normal use of the term in the field of nucleic acid biochemistry, and specifically to conventional Watson-Crick base-pairing. Thus a first nucleic acid molecule of sequence 5′-ACGA-3′ may form a duplex with a second nucleic acid molecule of sequence 5′-TCGT-3′ wherein the G of the first molecule will be positioned opposite the C of the second molecule and will hydrogen bond therewith. A first nucleic acid molecule of sequence 5′-ATGA-3′ may form a duplex with a second nucleic acid molecule of sequence 5′-TCGT-3′, wherein the T of the first molecule will mismatch with the G of the second molecule but will still be positioned opposite therewith and will act as a partner nucleotide. This principle applies to any nucleotide partner pair relationship disclosed herein, including partner pairs comprising universal nucleotides.

In all of the methods described herein a position in the first strand, and the opposite position in the second strand, is assigned the position number “n”. This position refers to the position of a nucleotide in the first strand of a scaffold polynucleotide which in the first extension/ligation reaction of any given synthesis cycle is the nucleotide position in the first strand of the scaffold polynucleotide which is occupied by or will be occupied by the first nucleotide of the predefined sequence upon its addition to the terminal end of the first strand in that cycle or in incorporation steps of subsequent cycles. Position “n” also refers to the position in the synthesis strand of a polynucleotide ligation molecule prior to the ligation step, which position is the nucleotide position which is occupied by the first nucleotide to be added to the terminal end of the first strand during the first extension/ligation reaction of any given synthesis cycle prior to ligation of the first polynucleotide ligation molecule to the scaffold polynucleotide.

Both the above-described position in the first strand and the opposite position in the second strand may be referred to as positon n.

Further details concerning the definition of position “n” are provided with reference to FIGS. 1 to 6 and the descriptions thereof in relation to the exemplary synthesis method versions of the invention and variants described in more detail herein.

Nucleotides and nucleotide analogues may preferably be provided as nucleoside triphosphates. Thus in any of the methods of the invention in order to synthesise DNA polynucleotides, nucleotides may be incorporated from 2′-deoxyribonucleoside-5′-O-triphosphates (dNTPs), e.g. via the action of a DNA polymerase enzyme or e.g. via the action of an enzyme having deoxynucleotidyl terminal transferase activity. In any of the methods of the invention in order to synthesise RNA polynucleotides, nucleotides may be incorporated ribonucleoside-5′-O-triphosphates (NTPs), e.g. via the action of a RNA polymerase enzyme or e.g. via the action of an enzyme having nucleotidyl terminal transferase activity. Triphosphates can be substituted by tetraphosphates or pentaphosphates (generally oligophosphate). These oligophosphates can be substituted by other alkyl or acyl groups:

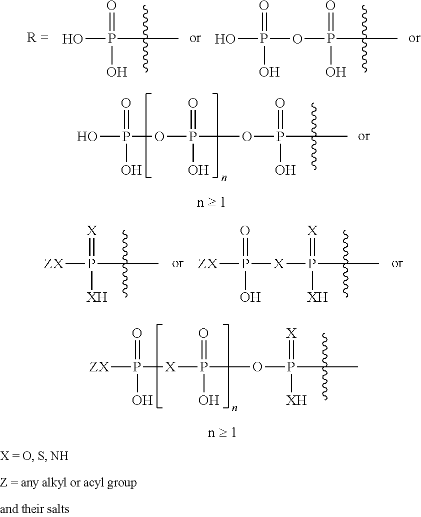

Exemplary methods of the invention use a universal nucleotide. A universal nucleotide may be used to define a cleavage site, as described further herein. A universal nucleotide may also be incorporated into the first and/or second strand of the scaffold polynucleotide as a component of the predefined nucleotide sequence if desired.

A universal nucleotide is one wherein the nucleobase will bond, e.g. hydrogen bond, to some degree to the nucleobase of any nucleotide of the predefined sequence. A universal nucleotide is preferably one which will bond, e.g. hydrogen bond, to some degree to nucleotides comprising the nucleosides adenosine (A), thymine (T), uracil (U), guanine (G) and cytosine (C). The universal nucleotide may bond more strongly to some nucleotides than to others. For instance, a universal nucleotide (I) comprising the nucleoside, 2′-deoxyinosine, will show a preferential order of pairing of I-C>I-A>I-G approximately =I-T.

Examples of possible universal nucleotides are inosines or nitro-indoles. The universal nucleotide preferably comprises one of the following nucleobases: hypoxanthine, 4-nitroindole, 5-nitroindole, 6-nitroindole, 3-nitropyrrole, nitroimidazole, 4-nitropyrazole, 4-nitrobenzimidazole, 5-nitroindazole, 4-aminobenzimidazole or phenyl (C6-aromatic ring. The universal nucleotide more preferably comprises one of the following nucleosides: 2′-deoxyinosine, inosine, 7-deaza-2′-deoxyinosine, 7-deaza-inosine, 2-aza-deoxyinosine, 2-aza-inosine, 4-nitroindole 2′-deoxyribonucleoside, 4-nitroindole ribonucleoside, 5-nitroindole 2′ deoxyribonucleoside, 5-nitroindole ribonucleoside, 6-nitroindole 2′ deoxyribonucleoside, 6-nitroindole ribonucleoside, 3-nitropyrrole 2′ deoxyribonucleoside, 3-nitropyrrole ribonucleoside, an acyclic sugar analogue of hypoxanthine, nitroimidazole 2′ deoxyribonucleoside, nitroimidazole ribonucleoside, 4-nitropyrazole 2′ deoxyribonucleoside, 4-nitropyrazole ribonucleoside, 4-nitrobenzimidazole 2′ deoxyribonucleoside, 4-nitrobenzimidazole ribonucleoside, 5-nitroindazole 2′ deoxyribonucleoside, 5-nitroindazole ribonucleoside, 4-aminobenzimidazole 2′ deoxyribonucleoside, 4-aminobenzimidazole ribonucleoside, phenyl C-ribonucleoside or phenyl C-2′-deoxyribosyl nucleoside.

Some examples of universal bases are shown below:

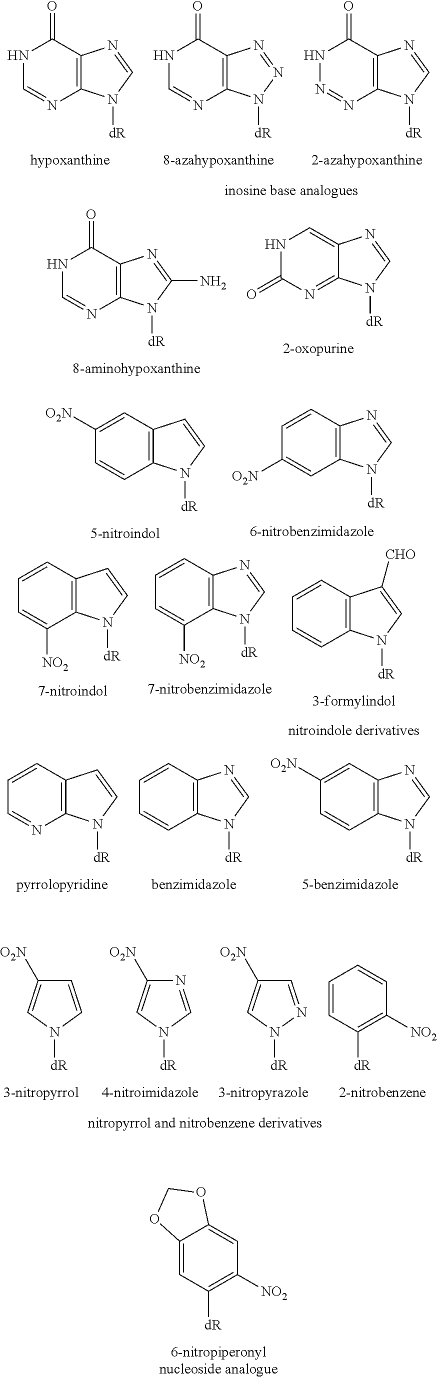

Universal nucleotides incorporating cleavable bases may also be used, including photo- and enzymatically-cleavable bases, some examples of which are shown below.

### Photocleavable Bases:

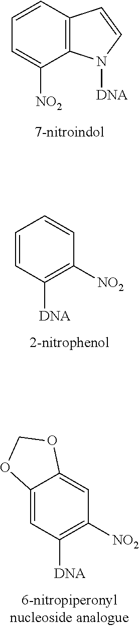

### Base Analogues Cleavable by Endonuclease III:

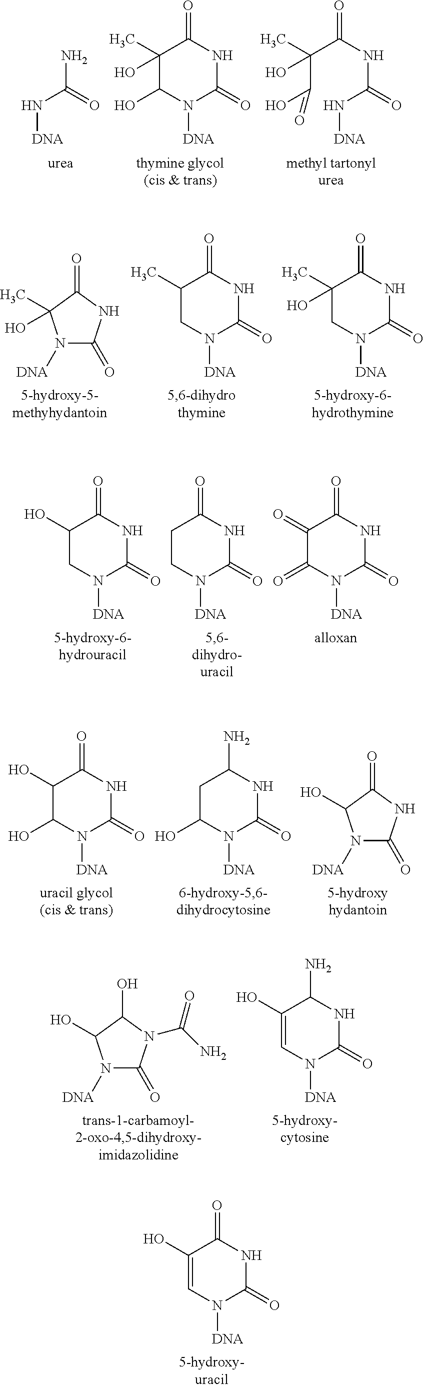

### Base Analogues Cleavable by Formamidopyrimidine DNA Glycosylase (Fpg):

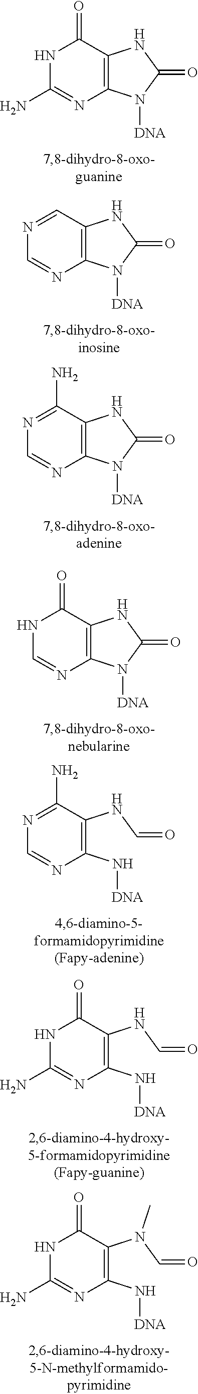

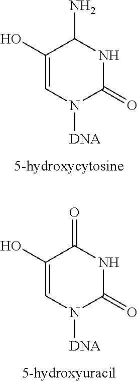

Base Analogues Cleavable by 8-Oxoguanine DNA Glycosylase (hOGG1):

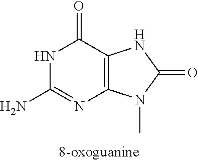

Base Analogues Cleavable by hNeill:

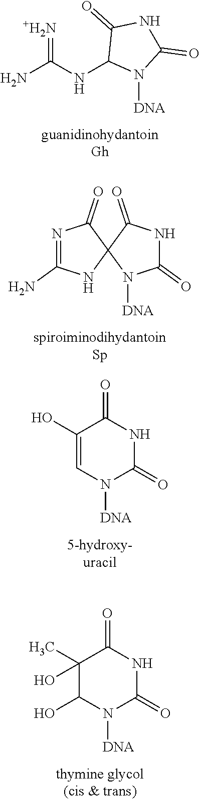

### Base Analogues Cleavable by Thymine DNA Glycosylase (TDG):

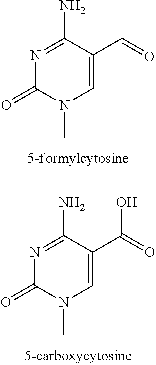

Base analogues cleavable by Human Alkyladenine DNA glycosylase (hAAG):

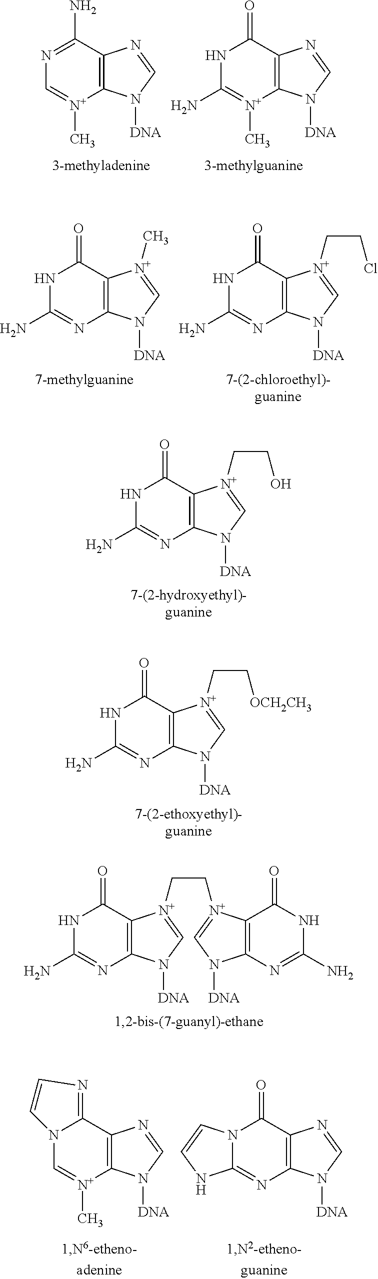

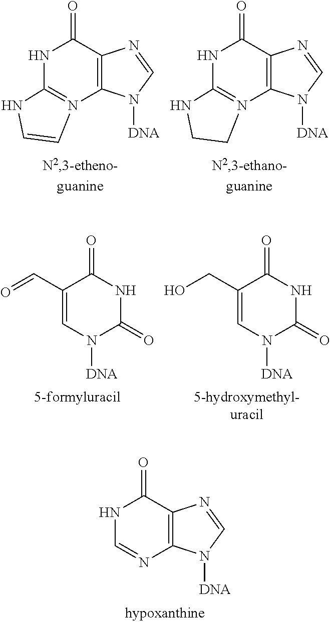

### Bases Cleavable by Uracil DNA Glycosylase:

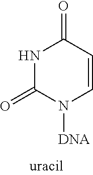

### Bases Cleavable by Human Single-Strand-Selective Monofunctional Uracil-DNA Glycosylase (SMUG1):

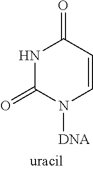

### Bases Cleavable by 5-Methylcytosine DNA Glycosylase (ROS1):

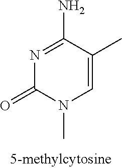

(see S. S. David, S. D. Williams Chemical reviews 1998, 98, 1221-1262 and M. I. Ponferrada-Marín, T. Roldán-Arjona, R. R. Ariza'Nucleic Acids Res 2009, 37, 4264-4274).

In any of the methods involving scaffold polynucleotides, the universal nucleotide most preferably comprises 2′-deoxyinosine.

Examples of epigenetic bases which may be incorporated using any of the synthesis methods described herein include the following:

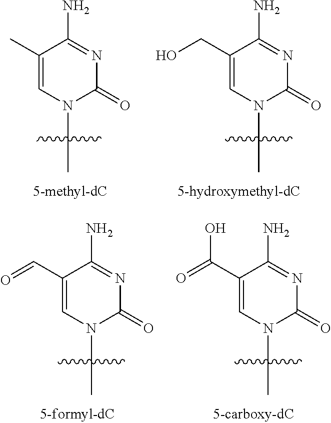

Examples of modified bases which may be incorporated using any of the synthesis methods described herein include the following:

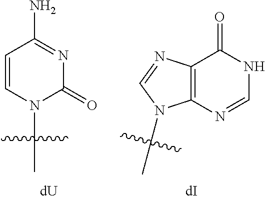 [SMILES: *n1ccc(N)nc1=O ; *n1cnc2c(=O)[nH]cnc21]

Examples of halogenated bases which may be incorporated using any of the synthesis methods described herein include the following:

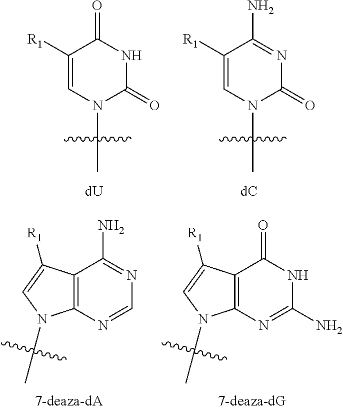

where R1=F, Cl, Br, I, alkyl, aryl, fluorescent label, aminopropargyl, aminoallyl.

Examples of amino-modified bases, which may be useful in e.g. attachment/linker chemistry, which may be incorporated using any of the synthesis methods described herein include the following:

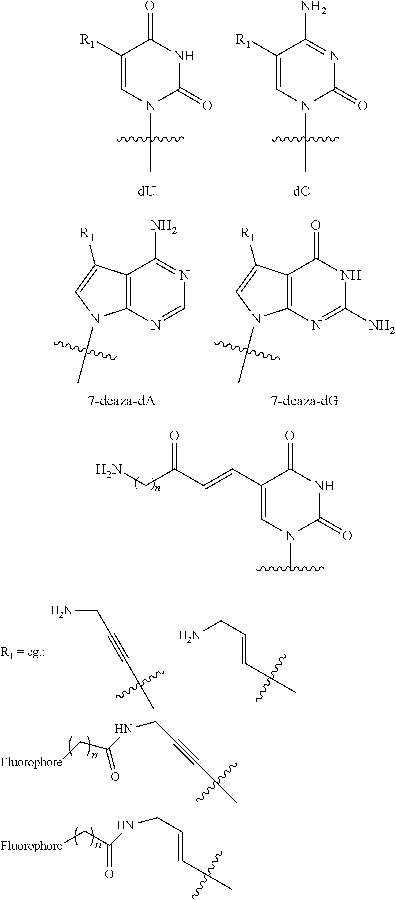

where base=A, T, G or C with alkyne or alkene linker.

Examples of modified bases, which may be useful in e.g. click chemistry, which may be incorporated using any of the synthesis methods described herein include the following:

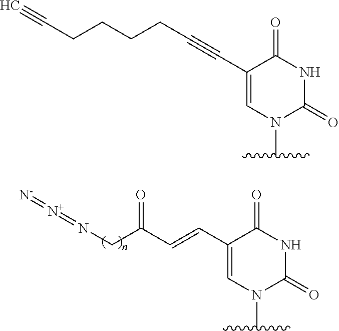

Examples of biotin-modified bases which may be incorporated using any of the synthesis methods described herein include the following:

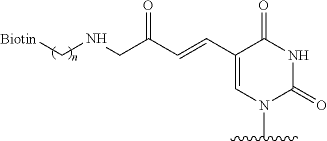

where base=A, T, G or C with alkyne or alkene linker.

Examples of bases bearing fluorophores and quenchers which may be incorporated using any of the synthesis methods described herein include the following:

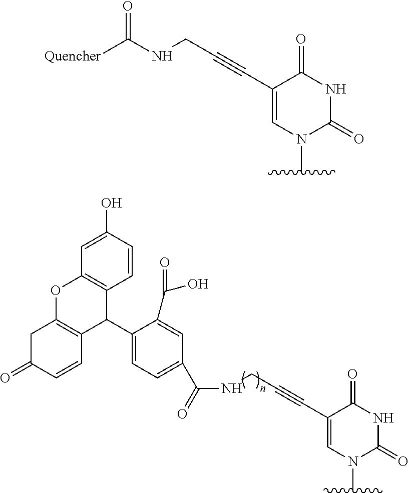

### Nucleotide-Incorporating Enzymes

In any of the methods described herein it may be desirable to copy one or both of the synthesised strands. For example, first and second strands of the scaffold polynucleotide may be separated following synthesis, one strand may be discarded and the other strand may be copied to provide a copied strand which has a nucleotide sequence which is complementary to the template strand which is copied. It may be desirable to copy both strands, such as in an amplification reaction e.g. PCR, or any alternative method as described further herein. In any such method a polymerase enzyme may be provided to copy the template strand.

In certain situations it may be desirable to incorporate nucleotides having attached reversible terminator groups, as described herein, in which case polymerase enzymes may be chosen based on their ability to incorporate modified nucleotides.

Thus the polymerase may be a modified polymerase having an enhanced ability to incorporate a nucleotide comprising a reversible terminator group compared to an unmodified polymerase. The polymerase is more preferably a genetically engineered variant of the native DNA polymerase from Thermococcus species 9° N, preferably species 9° N-7. Examples of modified polymerases are Therminator IX DNA polymerase and Therminator X DNA polymerase available from New England BioLabs. This enzyme has an enhanced ability to incorporate 3′-O-modified dNTPs. Examples of other polymerases that can be used for incorporation of reversible terminator dNTPs in any of the methods of the invention are Deep Vent (exo-), Vent

(Exo-), 9° N DNA polymerase, Therminator DNA polymerase, Therminator IX DNA polymerase, Therminator X DNA polymerase, Klenow fragment (Exo-), Bst DNA polymerase, Bsu DNA polymerase, Sulfolobus DNA polymerase I, and Taq Polymerase.

Examples of polymerases that can be used to copy a template strand are T3 RNA polymerase, T7 RNA polymerase, SP6 RNA polymerase, pol lambda, pol micro or 129 DNA polymerase.

To copy a template strand comprising DNA, a DNA polymerase may be used. Any suitable DNA polymerase may be used. The DNA polymerase may be for example Bst DNA polymerase full length, Bst DNA polymerase large fragment, B su DNA polymerase large fragment, E. coli DNA polymerase DNA Pol I large (Klenow) fragment, M-MuLV reverse transcriptase, phi29 DNA polymerase, Sulfolobus DNA polymerase IV, Taq DNA polymerase, T4 DNA polymerase, T7 DNA polymerase and enzymes having reverse transcriptase activity, for example M-MuLV reverse transcriptase. The DNA polymerase may lack 3′ to 5′ exonuclease activity. Any such suitable polymerase enzyme may be used. Such a DNA polymerase may be, for example, Bst DNA polymerase full length, Bst DNA polymerase large fragment, Bsu DNA polymerase large fragment, DNA Pol I large (Klenow) fragment (3′→5′ exo-), M-MuLV reverse transcriptase, Sulfolobus DNA polymerase IV, Taq DNA polymerase. The DNA polymerase may possess strand displacement activity. Any such suitable polymerase enzyme may be used. Such a DNA polymerase may be, for example, Bst DNA polymerase large fragment, Bsu DNA polymerase large fragment, DNA Pol I large (Klenow) fragment (3′→5′ exo-), M-MuLV reverse transcriptase, phi29 DNA polymerase. The DNA polymerase may lack 3′ to 5′ exonuclease activity and may possess strand displacement activity. Any such suitable polymerase enzyme may be used. Such a DNA polymerase may be, for example, Bst DNA polymerase large fragment, Bsu DNA polymerase large fragment, E. coli DNA polymerase DNA Pol I large (Klenow) fragment, M-MuLV reverse transcriptase. The DNA polymerase may lack 5′ to 3′ exonuclease activity. Any such suitable polymerase enzyme may be used. Such a DNA polymerase may be, for example, Bst DNA polymerase large fragment, Bsu DNA polymerase large fragment, DNA Pol I large (Klenow) fragment, DNA Pol I large (Klenow) fragment (3′→5′ exo-), M-MuLV reverse transcriptase, phi29 DNA polymerase, Sulfolobus DNA polymerase IV, T4 DNA polymerase, T7 DNA polymerase. The DNA polymerase may lack both 3′ to 5′ and 5′ to 3′ exonuclease activities and may possess strand displacement activity. Any such suitable polymerase enzyme may be used. Such a DNA polymerase may be, for example, Bst DNA polymerase large fragment, Bsu DNA polymerase large fragment, DNA Pol I large (Klenow) fragment (3′→5′ exo-), M-MuLV reverse transcriptase. The DNA polymerase may also be a genetically engineered variant. For example, the DNA polymerase may be a genetically engineered variant of the native DNA polymerase from Thermococcus species 9° N, such as species 9° N-7. One such example of a modified polymerase is Therminator IX DNA polymerase or Therminator X DNA polymerase available from New England BioLabs. Other engineered or variant DNA polymerases include Deep Vent (exo-), Vent (Exo-), 9° N DNA polymerase, Therminator DNA polymerase, Klenow fragment (Exo-), Bst DNA polymerase, Bsu DNA polymerase, Sulfolobus DNA polymerase I, and Taq Polymerase.

To copy a template strand comprising RNA, any suitable enzyme may be used. For example an RNA polymerase may be used. Any suitable RNA polymerase may be used. The RNA polymerase may be T3 RNA polymerase, T7 RNA polymerase, SP6 RNA polymerase, E. coli RNA polymerase holoenzyme.

In any of the methods described herein it may be desirable to perform one or more additional method steps to extend one or both of the strands of the scaffold polynucleotide as part of the process of extending the scaffold polynucleotide by the methods of the invention, e.g. before, during or after a process of extending one or both of the strands of the scaffold polynucleotide using the ligase-mediated methods of the invention. It may be desirable to extend one or both strands as part of a double stranded scaffold polynucleotide. It may also be desirable to extend one or both strands as single stranded polynucleotides following separation of the two strands of the scaffold polynucleotide. In any such additional method step the enzyme may have a terminal transferase activity, e.g. the enzyme may be a terminal nucleotidyl transferase, or terminal deoxynucleotidyl transferase, and wherein the scaffold polynucleotide is extended to form a polynucleotide molecule comprising DNA or RNA, preferably DNA. Any of these enzymes may be used in the methods of the invention wherein extension of a scaffold polynucleotide is required.

One such enzyme is a terminal nucleotidyl transferase enzyme, such as terminal deoxynucleotidyl transferase (TdT) (see e.g. Motea et al, 2010; Minhaz Ud-Dean, Syst. Synth. Biol., 2008, 2(3-4), 67-73). TdT is capable of catalysing the addition to a scaffold polynucleotide of a nucleotide molecule (nucleoside monophosphate) from a nucleoside triphosphate substrate (NTP or dNTP). TdT is capable of catalysing the addition of natural and non-natural nucleotides. It is also capable of catalysing the addition of nucleotide analogues (Motea et al, 2010). Pol lambda and pol micro enzymes may also be used (Ramadan K, et al., J. Mol. Biol., 2004, 339(2), 395-404), as may 129 DNA polymerase.

Techniques for the extension of a single-stranded polynucleotide molecule, both DNA and RNA, in the absence of a template by the action of a terminal transferase enzyme (e.g. terminal deoxynucleotidyl transferase; TdT) to create an artificially-synthesised single-stranded polynucleotide molecule has been extensively discussed in the art. Such techniques are disclosed in, for example, Patent application publications WO2016/034807, WO 2016/128731, WO2016/139477 and WO2017/009663, as well as US2014/0363852, US2016/0046973, US2016/0108382, and US2016/0168611. These documents describe the controlled extension of a single-stranded polynucleotide synthesis molecule by the action of TdT to create an artificially-synthesised single-stranded polynucleotide molecule. Extension by natural and non-natural/artificial nucleotides using such enzymes is described, as is extension by modified nucleotides, for example, nucleotides incorporating blocking groups. Any of the terminal transferase enzymes disclosed in these documents may be applied to methods of the present invention, as well as any enzyme fragment, derivative, analogue or functional equivalent thereof provided that the terminal transferase function is preserved in the enzyme.

Directed evolution techniques, conventional screening, rational or semi-rational engineering/mutagenesis methods or any other suitable methods may be used to alter any such enzyme to provide and/or optimise the required function. Any other enzyme which is capable of extending a single-stranded polynucleotide molecule portion, such as a molecule comprising DNA or RNA, or one strand of a blunt-ended molecule with a nucleotide without the use of a template may be used.

Thus in any of the methods defined herein a single stranded portion of a scaffold polynucleotide comprising DNA or blunt-ended double-stranded scaffold polynucleotide comprising DNA may be extended by an enzyme which has template-independent enzyme activity, such as template-independent polymerase or transferase activity. The enzyme may have nucleotidyl transferase enzyme activity, e.g. a deoxynucleotidyl transferase enzyme, such as terminal deoxynucleotidyl transferase (TdT), or an enzyme fragment, derivative, analogue or functional equivalent thereof. A scaffold polynucleotide extended by the action of such an enzyme comprises DNA.

A single stranded portion of a scaffold polynucleotide comprising RNA, or blunt-ended double-stranded scaffold polynucleotide comprising RNA may be extended by an enzyme which has nucleotidyl transferase enzyme (e.g. including TdT), or an enzyme fragment, derivative, analogue or functional equivalent thereof. A scaffold polynucleotide extended by the action of such an enzyme may comprise RNA. For the synthesis of a single stranded polynucleotide molecule comprising RNA, or a single stranded portion of a polynucleotide molecule comprising RNA, any suitable nucleotidyl transferase enzyme may be used. Nucleotidyl transferase enzymes such as poly (U) polymerase and poly(A) polymerase (e.g. from E. coli) are capable of template-independent addition of nucleoside monophosphate units to polynucleotide synthesis molecules. Any of these enzymes may be applied to methods described herein, as well as any enzyme fragment, derivative, analogue or functional equivalent thereof provided that the nucleotidyl transferase function is preserved in the enzyme. Directed evolution techniques, conventional screening, rational or semi-rational engineering/mutagenesis methods or any other suitable methods may be used to alter any such enzyme to provide and/or optimise the required function.

### Reversible Terminator Groups

If it is desirable, as an additional step, in any of the synthesis methods described herein, to incorporate into one or both strands of the scaffold polynucleotide by the action of a polymerase enzyme or a transferase enzyme, it may be further desirable to incorporate one or more such nucleotides as comprising one or more reversible blocking groups, also referred to as a reversible terminator group as described herein.

Such groups act to prevent further extension by the enzyme used to catalyse incorporation in a given synthesis cycle so that only one nucleotide may controllably be used to extend the scaffold polynucleotide, and thus non-specific nucleotide incorporation is prevented. Any functionality which achieves this effect may be used. Reversible blocking groups/reversible terminator groups attached to nucleotides and deblocking steps are preferred means for achieving this effect. However this effect may be achieved by alternative means as appropriate.

Any suitable reversible blocking group may be attached to a nucleotide to prevent further extension by the enzyme used to catalyse incorporation following the incorporation of a nucleotide into a polynucleotide strand in a given cycle and to limit incorporation into the strand to one nucleotide per step. The reversible blocking group may be a reversible terminator group which acts to prevent further extension by the enzyme used to catalyse incorporation. Examples of reversible terminators are provided below.

### Propargyl Reversible Terminators:

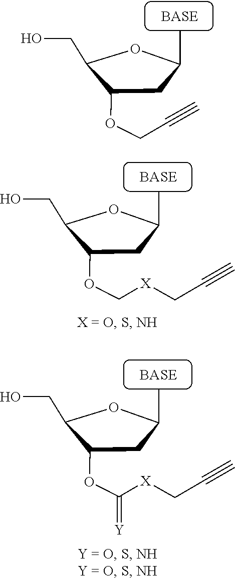

### Allyl Reversible Terminators:

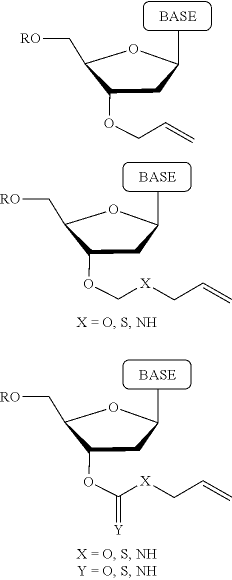

### Cyclooctene Reversible Terminators:

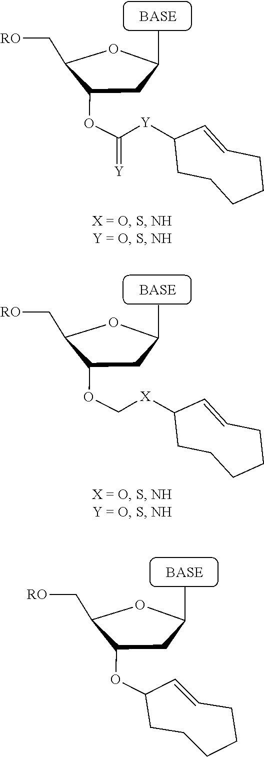

### Cyanoethyl Reversible Terminators:

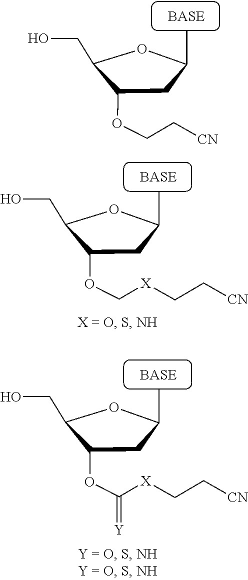

### Nitrobenzyl Reversible Terminators:

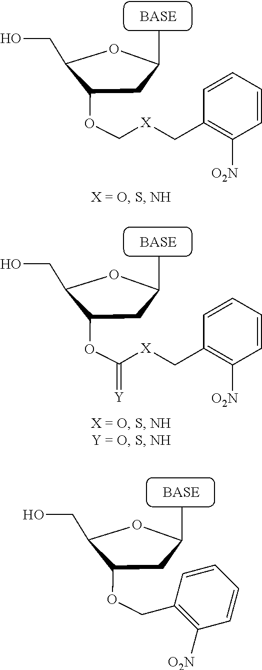

### Disulfide Reversible Terminators:

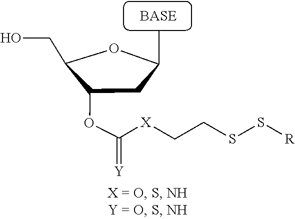

### Azidomethyl Reversible Therminators:

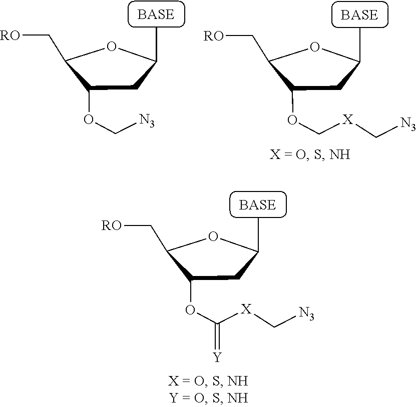

### Aminoalkoxy Reversible Therminators:

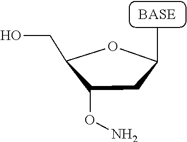

Nucleoside triphosphates with bulky groups attached to the base can serve as substitutes for a reversible terminator group on 3′-hydroxy group and can block further incorporation. This group can be deprotected by TCEP or DTT producing natural nucleotides.

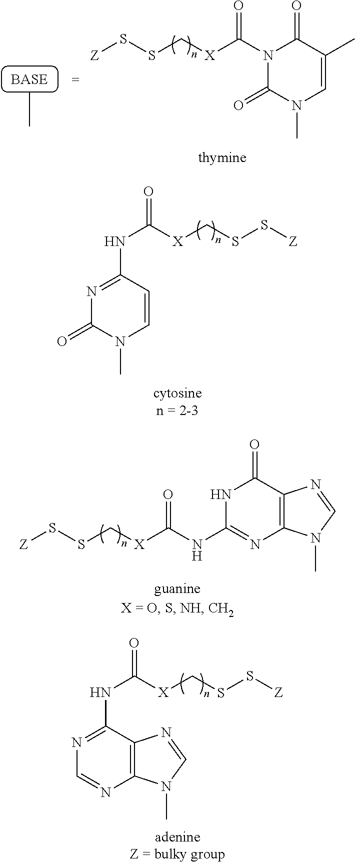

For synthesising DNA polynucleotides preferred modified nucleosides are 3′-O-modified-2′-deoxyribonucleoside-5′-O-triphosphate. For synthesising RNA polynucleotides preferred modified nucleosides are 3′-O-modified-ribonucleoside-5′-O-triphosphate.

Preferred modified dNTPs are modified dNTPs which are 3′-O-allyl-dNTPs and 3′-O-azidomethyl-dNTPs.

3′-O-allyl-dNTPs are shown below.

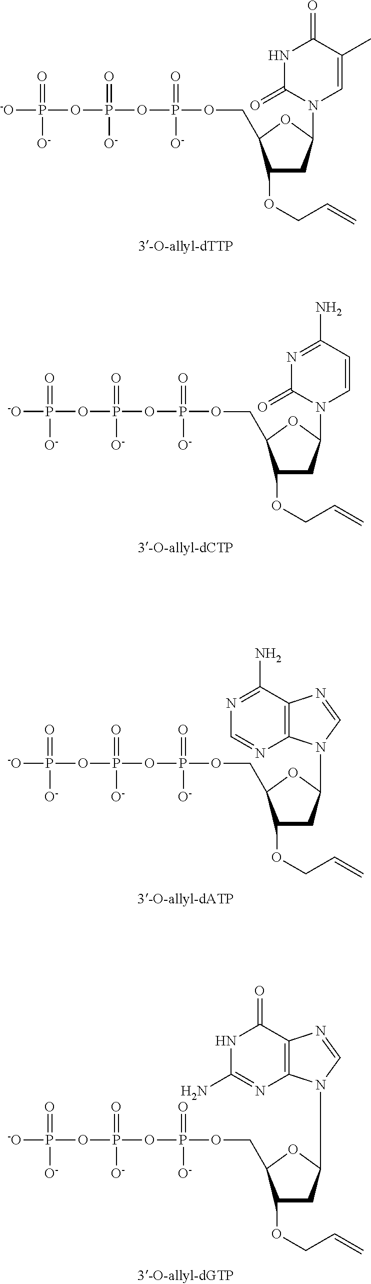

3′-O-azidomethyl-dNTPs are shown below.

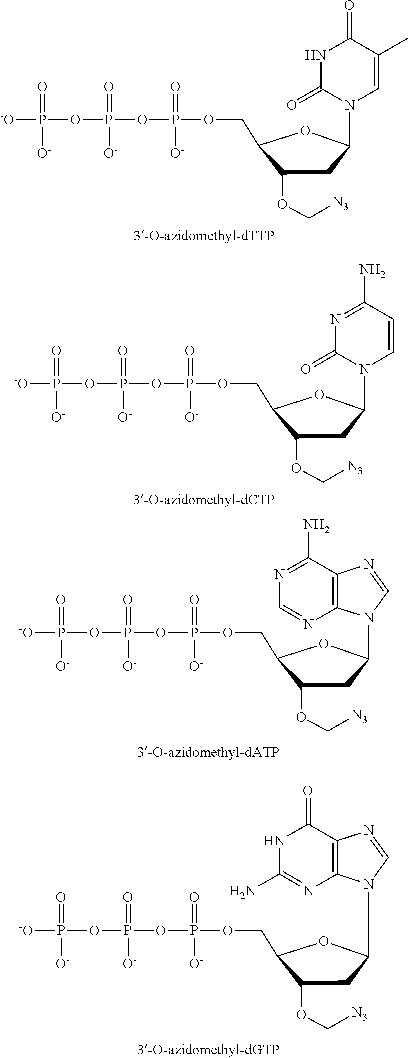

Following the incorporation of a nucleotide comprising a reversible blocking group, a deprotection or deblocking step may be performed. Such a step involves removal of the reversible blocking group (e.g. the reversible terminator group) by any suitable means, or otherwise reversing the functionality of the blocking/terminator group to inhibit further extension by the enzyme/polymerase.

Any suitable reagent may be used to remove the reversible terminator group during a deprotection step.

A preferred deprotecting reagent is tris(carboxyethyl)phosphine (TCEP). TCEP may be used to remove reversible terminator groups from 3′-O-allyl-nucleotides (in conjunction with Pd⁰) and 3′-O-azidomethyl-nucleotides following incorporation.

Examples of deprotecting reagents are provided below.

### Propargyl Reversible Terminators:

Treatment by Pd catalysts —Na₂PdCl₄, PdCl₂
Ligands can be used e.g.: Triphenylphosphine-3,3′,3″-trisulfonic acid trisodium salt.

### Allyl Reversible Terminators:

Treatment by Pd catalysts —Na₂PdCl₄, PdCl₂
Ligands can be used e.g.: Triphenylphosphine-3,3′,3″-trisulfonic acid trisodium salt.

### Azidomethyl Reversible Terminators:

Treatment by thiol (mercaptoethanol or dithiothreitol), or Tris (2-carboxyethyl)phosphine—TCEP.

### Cyanoethyl Reversible Terminators:

Treatment by fluoride—ammonium fluoride, tetrabutylammonium fluoride (TBAF).

### Nitrobenzyl Reversible Terminators:

Exposure to UV light

### Disulfide Reversible Terminators:

Treatment by thiol (mercaptoethanol or dithiothreitol), or Tris (2-carboxyethyl)phosphine—TCEP.

### Aminoalkoxy Reversible Terminators:

Treatment by nitrite (NO₂⁻, HNO₂) pH=5.5

A reversible blocking group (e.g., a reversible terminator group) can be removed by a step performed immediately after the incorporation step, provided that unwanted reagent from the incorporation step is removed to prevent further incorporation following removal of the reversible terminator group.

### Polynucleotide Ligation Molecule

As described further herein, all exemplary methods of the invention involve first and second ligation/extension steps wherein in each case one or more nucleotides of predefined sequence are attached to a strand of the scaffold polynucleotide by the action of an enzyme having ligase activity. In such methods the selection of the configuration and structure of the polynucleotide ligation molecule will depend upon the particular method employed. The polynucleotide ligation molecule comprises a synthesis strand as described herein and a helper strand as described herein. The polynucleotide ligation molecule comprises a complementary ligation end at one end of the molecule. The complementary ligation end of the polynucleotide ligation molecule will be ligated to a terminal end of the scaffold polynucleotide.

The complementary ligation end of the polynucleotide ligation molecule is provided with a non-ligatable terminal nucleotide in the helper strand. This prevents ligation of the helper strand to the first or second strand of the scaffold polynucleotide and thus creates a single-strand break between the helper strand and the first or second strand of the scaffold polynucleotide following ligation. If the terminal nucleotide of the helper strand is at the 3′ end of the helper strand, the nucleotide may be provided as a non-ligatable 2′,3′-dideoxynucleotide or a 2′-deoxynucleotide, or any other suitable non-ligatable nucleotide. If the terminal nucleotide of the helper strand is at the 5′ end of the helper strand, the nucleotide may be provided without a phosphate group, i.e. it may be provided as a nucleoside. Alternatively a 5′-protected nucleoside, a nucleoside with a non-ligatable group at the 5′ position, such as 5′-deoxynucleoside or a 5′-aminonucleoside, or any other suitable non-ligatable nucleotide or nucleoside may be used. Alternative means for preventing ligation could be employed. For example blocking moieties could be attached to the terminal nucleotide in the helper strand.

The complementary ligation end of the polynucleotide ligation molecule is provided with a ligatable terminal nucleotide in the synthesis strand adjacent the non-ligatable terminal nucleotide in the helper strand. The ligatable terminal nucleotide of the synthesis strand of the first and second polynucleotide ligation molecule is the first nucleotide of the predefined sequence to be incorporated respectively into the first and second strand of the scaffold molecule by the action of a ligase enzyme. The complementary ligation end of the polynucleotide ligation molecule is also provided with a universal nucleotide in the synthesis strand. The exact positioning of the universal nucleotide in the synthesis strand relative to the ligatable terminal nucleotide of the synthesis strand will depend upon the specific reaction chemistry employed as will be apparent from the descriptions of the specific exemplary method versions of the invention and variants thereof.

### Helper Strand

A helper strand is provided in the polynucleotide ligation molecule to facilitate ligation of the synthesis strand of the polynucleotide ligation molecule to the scaffold polynucleotide at the ligation step. A helper strand may also facilitate binding of cleavage enzyme(s) at the cleavage step. The helper strand may be omitted, provided that alternative means are provided to ensure binding of cleavage enzyme(s) at the cleavage step and to ensure ligation at the ligation step, if necessary. In preferred methods of the invention the polynucleotide ligation molecule is provided with a helper strand.

There are no special requirements for the parameters of length, sequence and structure of the helper strand, provided that the helper strand is suitable to facilitate binding of ligase and cleavage enzyme(s) as necessary.

The helper strand may comprise nucleotides, nucleotide analogues/derivatives and/or non-nucleotides.

Preferably, within the region of sequence of the helper strand mismatches with the synthesis strand should be avoided, GC- and AT-rich regions should be avoided, and in addition regions of secondary structure such as hairpins or bulges should be avoided.

The length of the helper strand may be 10 bases or more. Optionally, the length of the helper strand may be 15 bases or more, preferably 30 bases or more. However, the length of the helper strand may be varied, provided that the helper strand is capable of facilitating cleavage and/or ligation.

The helper strand must be hybridized to the corresponding region of the synthesis strand. It is not essential that the entirety of the helper strand is hybridized to the corresponding region of the support strand, provided that the helper strand can facilitate binding of ligase enzyme at the ligation step and/or binding of cleavage enzyme(s) at the cleavage step. Thus, mismatches between the helper strand and the corresponding region of the synthesis strand can be tolerated. The helper strand may be longer than the corresponding region of the synthesis strand. The synthesis strand may extend beyond the region which corresponds with the helper strand in the direction distal to the complementary ligation end. The helper strand may be connected to the corresponding region of the synthesis strand, e.g. via a hairpin.

The helper strand may be hybridized to the synthesis strand of the polynucleotide ligation molecule such that when the polynucleotide ligation molecule is ligated to the scaffold polynucleotide the terminal nucleotide of the helper strand at the site of the nick occupies the next sequential nucleotide position in the synthesis strand relative to the terminal nucleotide of the relevant strand of the scaffold polynucleotide at the site of the nick. Thus in this configuration there are no nucleotide position gaps between the helper strand and the relevant strand of the scaffold polynucleotide. The helper strand and the relevant strand of the scaffold polynucleotide will nevertheless be physically separated due to the presence of the single-stranded break or nick.

The nucleotide in the helper strand which pairs with the universal nucleotide may be any suitable nucleotide. Preferably, pairings which are likely to distort the helical structure of the molecule should be avoided. Preferably cytosine acts as a partner for the universal nucleotide. In a particularly preferred embodiment the universal nucleotide is inosine, or an analogue, variant or derivative thereof, and the partner nucleotide for the universal nucleotide in the helper strand is cytosine.

### Removal of Helper Strand

In any of the synthesis methods of the invention described herein, prior to a cleavage step the helper strand provided by the polynucleotide ligation molecule may be removed from the ligated scaffold polynucleotide.

The helper strand may be removed from the scaffold polynucleotide by any suitable means including, but not limited to: (i) heating the scaffold polynucleotide to a temperature of about 80° C. to about 95° C. and separating the helper strand portion from the scaffold polynucleotide, (ii) treating the scaffold polynucleotide with urea solution, such as 8M urea and separating the helper strand portion from the scaffold polynucleotide, (iii) treating the scaffold polynucleotide with formamide or formamide solution, such as 100% formamide and separating the helper strand portion from the scaffold polynucleotide, or (iv) contacting the scaffold polynucleotide with a single-stranded polynucleotide molecule which comprises a region of nucleotide sequence which is complementary with the sequence of the helper strand, thereby competitively inhibiting the hybridisation of the helper strand to the scaffold polynucleotide.

In methods wherein the helper strand is removed from the scaffold polynucleotide after the step of ligating the double-stranded polynucleotide ligation molecule to the cleaved scaffold polynucleotide and before the step of cleavage of the scaffold polynucleotide, the cleavage step will comprise cleaving the support strand in the absence of a double-stranded region provided by the helper strand. Any suitable enzyme may be chosen for performing such a cleavage step, such as selected from any suitable enzyme disclosed herein.

The appropriate structure of a polynucleotide ligation molecule can readily be ascertained by reference to the exemplary methods of the invention described herein and depictions of the same in the Figures (FIGS. 1 to 10).

### Ligation and Ligase Enzymes

In methods of the invention which involve a ligation step, ligation may be achieved using any suitable means. Preferably, the ligation step will be performed by a ligase enzyme. The ligase may be a modified ligase with enhanced activity for single-base overhang substrates. The ligase may be a T3 DNA ligase or a T4 DNA ligase. The ligase may a blunt TA ligase. For example a blunt TA ligase is available from New England BioLabs (NEB). This is a ready-to-use master mix solution of T4 DNA Ligase, ligation enhancer, and optimized reaction buffer specifically formulated to improve ligation and transformation of both blunt-end and single-base overhang substrates. Molecules, enzymes, chemicals and methods for ligating (joining) single- and double-stranded polynucleotides are well known to the skilled person.

### Cleavage of Scaffold Polynucleotide

In methods requiring the presence of scaffold polynucleotides and steps of cleavage, the selection of the reagent to perform the cleavage step will depend upon the particular method employed. The cleavage site is defined by the specific position of the universal nucleotide in the synthesis strand. Configuration of the desired cleavage site and selection of the appropriate cleavage reagent will therefore depend upon the specific chemistry employed in the method, as will readily be apparent by reference to the exemplary methods described herein.

Some examples of DNA cleaving enzymes recognizing modified bases are shown in the Table below.

| DNA | | | Termini created from the | |
|---|---|---|---|---|
| glycosylase/ | Main | | cleavage | |
| Endonuclease | substrate | Cleavage site | 5′-end | 3′-end |
| APE1 | AP site | 1^{st} | Deoxyribose- | OH |
| | | phosphodiester | 5′-phosphate | |
| | | bond 5′ to the | | |
| | | lesion | | |
| Endonuclease | AP site, | 1^{st} | phosphate | 3′-phospho- |
| III | thymine glycol | phosphodiester | | α, β- |
| | | bond 3′ to the | | unsaturated |
| | | lesion | | aldehyde |
| Endonuclease | AP site | 1^{st} | Deoxyribose- | OH |
| IV | | phosphodiester | 5′-phosphate | |
| | | bond 5′ to the | | |
| | | lesion | | |
| Endonuclease | Inosine | 2^{nd} | phosphate | OH |
| V | | phosphodiester | | |
| | | bond 3′ to the | | |
| | | lesion | | |
| Endonuclease | AP site, | 1^{st} | phosphate | phosphate |
| VIII | thymine glycol | phosphodiester | | |
| | | bond 5′ and 3′ | | |
| | | to the lesion | | |
| FpG | 8-oxoguanine | 1^{st} | phosphate | phosphate |
| | | phosphodiester | | |
| | | bond 5′ and 3′ | | |
| | | to the lesion | | |
| hOGGI | 8-oxoguanine | 1^{st} | phosphate | 3′-phospho- |
| | | phosphodiester | | α, β- |
| | | bond 3′ to the | | unsaturated |
| | | lesion | | aldehyde |
| hNeill | Oxidized | 1^{st} | phosphate | phosphate |
| | purines | phosphodiester | | |
| | | bond 5′ and 3′ | | |
| | | to the lesion | | |
| ROS1 | 5- | 1^{st} | phosphate | phosphate |
| | methylcytosine | phosphodiester | | |
| | | bond 5′ and 3′ | | |
| | | to the lesion | | |

| | | | |
|---|---|---|---|
| Uracil DNA | Uracil | N-glycosidic | AP site (no break) |
| glycosylase | | bond | |
| hSMUG | Uracil | N-glycosidic | AP site (no break) |
| | | bond | |
| hAAG | Inosine | N-glycosidic | AP site (no break) |
| | | bond | |

### Synthesis Strand

In methods of synthesising a polynucleotide or oligonucleotide described herein including, but not limited to, synthesis method versions 1 and 2 of the invention and variants thereof as described in FIGS. 1 to 6 and further herein, the scaffold polynucleotide is provided with a synthesis strand. The synthesis strand comprises a primer strand portion. During cycles of synthesis each new second nucleotide of the predefined sequence is incorporated into the synthesis strand by extension of the primer strand portion, the first nucleotide of the predefined sequence being incorporated into the support strand. An enzyme, such as a polymerase enzyme or enzyme having terminal transferase activity, can be used to catalyse incorporation/addition of each new second nucleotide. Each newly-incorporated second nucleotide of the predefined sequence will act as the terminal nucleotide of the primer strand portion for use in priming incorporation in the next incorporation step. Thus in any given cycle of synthesis the primer strand portion of the synthesis strand will comprise sufficient polynucleotide sequence to allow priming by the appropriate enzyme. In certain embodiments, described further herein, in a given cycle of synthesis a second nucleotide of the predefined sequence is incorporated into the synthesis strand followed by incorporation into the synthesis strand of one or more further nucleotides. In such embodiments the second nucleotide of the predefined sequence and further nucleotides comprise a reversible terminator group and the methods additionally comprise steps of removing the reversible terminator group from the nucleotide following incorporation and prior to incorporation of the next nucleotide.

The terms “incorporation”, “extension” and “addition” of a nucleotide are intended to have the same meaning herein.

### First and Second Strands of a Scaffold Polynucleotide

The first and second strands of a scaffold polynucleotide should be suitable to allow an enzyme having ligase activity to catalyse the ligation of a polynucleotide ligation molecule to the scaffold polynucleotide as described further herein. In addition, if further extension of either strand is desired, the first and second strands of a scaffold polynucleotide should be suitable to allow an enzyme, such as a polymerase enzyme or enzyme having terminal transferase activity, to initiate synthesis, i.e. catalyse the addition of a new nucleotide at the terminal end of the scaffold polynucleotide.

There are no special requirements for the parameters of length, sequence and structure of the first or second strand of the scaffold polynucleotide, provided that the first and second strands of the scaffold polynucleotide are suitable to facilitate ligation as described further herein, and to prime new polynucleotide synthesis as described further herein if desired.

The first and second strands may comprise nucleotides, nucleotide analogues/derivatives and/or non-nucleotides.

The skilled person is readily able to construct a scaffold polynucleotide comprising first and second strands which will be suitable to facilitate ligation as described further herein and which are capable of priming new polynucleotide synthesis as described further herein if desired. At the end of the scaffold polynucleotide which is to be extended mismatches with the support strand should be avoided, GC- and AT-rich regions should be avoided, and in addition regions of secondary structure such as hairpins or bulges which might interfere with ligation and/or other extension should be avoided.

Prior to the initiation of synthesis the length of the first and second strands of the scaffold polynucleotide can be chosen by the skilled person depending on preference and the ligase enzyme to be used.

The first strand must be hybridized to the corresponding region of the second strand. It is not essential that the entirety of the first strand is hybridized to the corresponding region of the second strand, provided that first and second strands are suitable for ligation as described herein, or capable of priming new polynucleotide synthesis as described further herein if desired. Thus, mismatches between the first strand and the corresponding region of the second strand can be tolerated to a degree. Preferably, the region of sequence of the first and second strands at the end of the scaffold polynucleotide to be extended should comprise nucleobases which are complementary to corresponding nucleobases in the support strand.

The first strand may be connected to the corresponding region of the second strand at the end of the scaffold polynucleotide which is not to be extended, e.g. via a hairpin.

### Synthetic Polynucleotide

The polynucleotide having a predefined sequence synthesised according to the methods described herein is double-stranded. The synthesised polynucleotide overall is double-stranded and wherein the first strand is attached to the second strand by hybridization. Mismatches and regions of non-hybridization may be tolerated, provided that overall the first strand is attached to the second strand by hybridization.

Hybridisation may be defined by moderately stringent or stringent hybridisation conditions. A moderately stringent hybridisation condition uses a prewashing solution containing 5× sodium chloride/sodium citrate (SSC), 0.5% SDS, 1.0 mM EDTA (pH 8.0), hybridisation buffer of about 50% formamide, 6×SSC, and a hybridisation temperature of 55° C. (or other similar hybridisation solutions, such as one containing about 50% formamide, with a hybridisation temperature of 42° C.), and washing conditions of 60° C., in 0.5×SSC, 0.1% SDS. A stringent hybridisation condition hybridises in 6×SSC at 45° C., followed by one or more washes in 0.1×SSC, 0.2% SDS at 68° C.

The double-stranded polynucleotide having a predefined sequence synthesised according to the methods described herein may be retained as a double-stranded polynucleotide. Alternatively the two strands of the double-stranded polynucleotide may be separated to provide a single-stranded polynucleotide having a predefined sequence. Conditions that permit separation of two strands of a double-stranded polynucleotide (melting) are well-known in the art (for example, Sambrook et al., 2001, Molecular Cloning: a laboratory manual, 3rd edition, Cold Spring Harbour Laboratory Press; and

Current Protocols in Molecular Biology, Greene Publishing and Wiley-Interscience, New York (1995)).

The double-stranded polynucleotide having a predefined sequence synthesised according to the methods described herein may be amplified following synthesis. Any region of the double-stranded polynucleotide may be amplified. The whole or any region of the double-stranded polynucleotide may be amplified together with the whole or any region of the scaffold polynucleotide. Conditions that permit amplification of a double-stranded polynucleotide are well-known in the art (for example, Sambrook et al., 2001, Molecular Cloning: a laboratory manual, 3rd edition, Cold Spring Harbour Laboratory Press; and Current Protocols in Molecular Biology, Greene Publishing and Wiley-Interscience, New York (1995)). Thus any of the synthesis methods described herein may further comprise an amplification step wherein the double-stranded polynucleotide having a predefined sequence, or any region thereof, is amplified as described above. Amplification may be performed by any suitable method, such as polymerase chain reaction (PCR), polymerase spiral reaction (PSR), loop mediated isothermal amplification (LAMP), nucleic acid sequence based amplification (NASBA), self-sustained sequence replication (3SR), rolling circle amplification (RCA), strand displacement amplification (SDA), multiple displacement amplification (MDA), ligase chain reaction (LCR), helicase dependent amplification (HDA), ramification amplification method (RAM) etc. Preferably, amplification is performed by polymerase chain reaction (PCR).

The double-stranded or single-stranded polynucleotide having a predefined sequence synthesised according to the methods described herein can be any length. For example, the polynucleotides can be at least 10, at least 50, at least 100, at least 150, at least 200, at least 250, at least 300, at least 350, at least 400, at least 450 or at least 500 nucleotides or nucleotide pairs in length. For example, the polynucleotides may be from about 10 to about 100 nucleotides or nucleotide pairs, about 10 to about 200 nucleotides or nucleotide pairs, about 10 to about 300 nucleotides or nucleotide pairs, about 10 to about 400 nucleotides or nucleotide pairs and about 10 to about 500 nucleotides or nucleotide pairs in length. The polynucleotides can be up to about 1000 or more nucleotides or nucleotide pairs, up to about 5000 or more nucleotides or nucleotide pairs in length or up to about 100000 or more nucleotides or nucleotide pairs in length.

### RNA Synthesis

Methods described for DNA synthesis may be adapted for the synthesis of RNA. In one adaptation the synthesis steps described for synthesis method versions of the invention 1 to 6 and variants thereof may be adapted.

Nucleotides may be incorporated as ribonucleoside-5′-O-triphosphates (NTPs).

Thus the descriptions relating to synthesis method versions of the invention 1 to 6 and variants thereof may be applied mutatis mutandis for RNA synthesis as adapted as required using relevant enzymes which may have the capability of ligating RNA strands. Alternatively, DNA strands may be converted to RNA strands using enzymes and methods for the transcription of DNA sequences into RNA sequences.

### Solid Phase Synthesis

Synthetic polynucleotides produced in accordance with the synthesis methods of the invention may preferably be synthesised using solid phase or reversible solid phase techniques. A variety of such techniques is known in the art and may be used. Before initiating synthesis of a new double-stranded polynucleotide of predefined sequence, scaffold polynucleotides may be immobilized to a surface e.g. a planar surface such as glass, a gel-based material, or the surface of a microparticle such as a bead or functionalised quantum dot. The material comprising the surface may itself be bound to a substrate. For example, scaffold polynucleotides may be immobilized to a gel-based material such as e.g. polyacrylamide, and wherein the a gel-based material is bound to a supporting substrate such as glass.

Polynucleotides may be immobilized or tethered to surfaces directly or indirectly. For example they may be attached directly to surfaces by chemical bonding. They may be indirectly tethered to surfaces via an intermediate surface, such as the surface of a microparticle or bead e.g. as in SPRI or as in electrowetting systems, as described below. Cycles of synthesis may then be initiated and completed whilst the scaffold polynucleotide incorporating the newly-synthesised polynucleotide is immobilized.

In such methods a double-stranded scaffold polynucleotide may be immobilized to a surface prior to the incorporation of the first nucleotide of the predefined sequence. Such an immobilized double-stranded scaffold polynucleotide may therefore act as an anchor to tether the double-stranded polynucleotide of the predefined sequence to the surface during and after synthesis.

Only one strand of such a double-stranded anchor/scaffold polynucleotide may be immobilized to the surface at the same end of the molecule. Alternatively both strands of a double-stranded anchor/scaffold polynucleotide may each be immobilized to the surface at the same end of the molecule. A double-stranded anchor/scaffold polynucleotide may be provided with each strand connected at adjacent ends, such as via a hairpin loop at the opposite end to the site of initiation of new synthesis, and connected ends may be immobilized on a surface (for example as depicted schematically in FIG. 12).

In methods involving a scaffold polynucleotide, as described herein, the scaffold polynucleotide may be attached to a surface prior to the incorporation of the first nucleotide in the predefined sequence. Thus the synthesis strand comprising the primer strand portion and the portion of the support strand hybridized thereto may both be separately attached to a surface, as depicted in FIGS. 12(a) and (c). The synthesis strand comprising the primer strand portion and the portion of the support strand hybridized thereto may be connected at adjacent ends, such as via a hairpin loop, e.g. at the opposite end to the site of initiation of new synthesis, and connected ends may be tethered to a surface, as depicted in FIGS. 12(b) and (d). One or other of the synthesis strand comprising the primer strand portion and the portion of the support strand hybridized thereto may be attached separately to a surface, as depicted in FIG. 12(e) to (h). Preferably the synthesis strand comprising the primer strand portion and the portion of the support strand hybridized thereto is attached to a surface.

### Solid Phase Synthesis on Planar Surfaces

Before initiating synthesis of a new double-stranded polynucleotide of predefined sequence synthetic anchor/scaffold polynucleotides can be synthesised by methods known in the art, including those described herein, and tethered to a surface.

Pre-formed polynucleotides can be tethered to surfaces by methods commonly employed to create nucleic acid microarrays attached to planar surfaces. For example, anchor/scaffold polynucleotides may be created and then spotted or printed onto a planar surface. Anchor/scaffold polynucleotides may be deposited onto surfaces using contact printing techniques. For example, solid or hollow tips or pins may be dipped into solutions comprising pre-formed scaffold polynucleotides and contacted with the planar surface. Alternatively, oligonucleotides may be adsorbed onto micro-stamps and then transferred to a planar surface by physical contact. Non-contact printing techniques include thermic printing or piezoelectric printing wherein sub-nanolitre size microdroplets comprising pre-formed scaffold polynucleotides may be ejected from a printing tip using methods similar to those used in inkjet and bubblejet printing.

Single-stranded oligonucleotides may be synthesised directly on planar surfaces such as using so-called “on-chip” methods employed to create microarrays. Such single-stranded oligonucleotides may then act as attachment sites to immobilize pre-formed anchor/scaffold polynucleotides.

On-chip techniques for generating single-stranded oligonucleotides include photolithography which involves the use of UV light directed through a photolithographic mask to selectively activate a protected nucleotide allowing for the subsequent incorporation of a new protected nucleotide. Cycles of UV-mediated deprotection and coupling of pre-determined nucleotides allows the in situ generation of an oligonucleotide having a desired sequence. As an alternative to the use of a photolithographic mask, oligonucleotides may be created on planar surfaces by the sequential deposition of nucleobases using inkjet printing technology and the use of cycles of coupling, oxidation and deprotection to generate an oligonucleotide having a desired sequence (for a review see Kosuri and Church, Nature Methods, 2014, 11, 499-507).

In any of the synthesis methods described herein, including methods involving reversible immobilisation as described below, surfaces can be made of any suitable material. Typically a surface may comprise silicon, glass or polymeric material. A surface may comprise a gel surface, such as a polyacrylamide surface, such as about 2% polyacrylamide, optionally a polyacrylamide surface derived using N-(5-bromoacetamidylpentyl) acrylamide (BRAPA), preferably the polyacrylamide surface is coupled to a solid support, such as glass.

### Reversible Immobilization

Synthetic polynucleotides having a predefined sequence can be synthesised in accordance with the invention using binding surfaces and structures, such as microparticles and beads, which facilitate reversible immobilization. Solid phase reversible immobilization (SPRI) methods or modified methods are known in the art and may be employed (e.g. see DeAngelis M. M. et al. (1995) Solid-Phase Reversible Immobilization for the Isolation of PCR Products, Nucleic Acids Research, 23(22): 4742-4743).

Surfaces can be provided in the form of microparticles, such as paramagnetic beads. Paramagnetic beads can agglomerate under the influence of a magnetic field. For example, paramagnetic surfaces can be provided with chemical groups, e.g. carboxyl groups, which in appropriate attachment conditions will act as binding moieties for nucleic acids, as described in more detail below. Nucleic acids can be eluted from such surfaces in appropriate elution conditions. Surfaces of microparticles and beads can be provided with UV-sensitive polycarbonate. Nucleic acids can be bound to the activated surface in the presence of a suitable immobilization buffer.

Microparticles and beads may be allowed to move freely within a reaction solution and then reversibly immobilized, e.g. by holding the bead within a microwell or pit etched into a surface. A bead can be localised as part of an array e.g. by the use of a unique nucleic acid “barcode” attached to the bead or by the use of colour-coding.

Thus before initiating synthesis of a new double-stranded polynucleotide of predefined sequence, anchor/scaffold polynucleotides in accordance with the invention can be synthesised and then reversibly immobilized to such binding surfaces. Polynucleotides synthesised by methods of the invention can be synthesised whilst reversibly immobilized to such binding surfaces.

### Microfluidic Techniques and Systems

The surface may be part of an electrowetting-on-dielectric system (EWOD). EWOD systems provide a dielectric-coated surface which facilitates microfluidic manipulation of very small liquid volumes in the form of microdroplets (e.g. see Chou, W-L., et al. (2015) Recent Advances in Applications of Droplet Microfluidics, Micromachines, 6: 1249-1271). Droplet volumes can programmably be created, moved, partitioned and combined on-chip by electrowetting techniques. Thus electrowetting systems provide alternative means to reversibly immobilize polynucleotides during and after synthesis.

Polynucleotides having a predefined sequence may be synthesised in solid phase by methods described herein, wherein polynucleotides are immobilized on an EWOD surface and required steps in each cycle facilitated by electrowetting techniques. For example, in methods involving scaffold polynucleotides and requiring incorporation, cleavage, ligation and deprotection steps, reagents required for each step, as well as for any required washing steps to remove used and unwanted reagent, can be provided in the form of microdroplets transported under the influence of an electric field via electrowetting techniques.

Other microfluidic platforms are available which may be used in the synthesis methods of the invention. For example, the emulsion-based microdroplet techniques which are commonly employed for nucleic acid manipulation can be used. In such systems microdroplets are formed in an emulsion created by the mixing of two immiscible fluids, typically water and an oil. Emulsion microdroplets can be programmably be created, moved, partitioned and combined in microfluidic networks. Hydrogel systems are also available. In any of the synthesis methods described herein microdroplets may be manipulated in any suitable compatible system, such as EWOD systems described above and other microfluidic systems, e.g. microfluidic systems comprising architectures based on components comprising elastomeric materials.

Microdroplets may be of any suitable size, provided that they are compatible with the synthesis methods herein. Microdroplet sizes will vary depending upon the particular system employed and the relevant architecture of the system. Sizes may thus be adapted as appropriate. In any of the synthesis methods described herein droplet diameters may be in the range from about 150 nm to about 5 mm. Droplet diameters below 1 μm may be verified by means known in the art, such as by techniques involving capillary jet methods, e.g. as described in Gañán-Calvo et al. (Nature Physics, 2007, 3, pp737-742)

### Sequencing of Intermediate or Final Synthesis Products.

The intermediate products of synthesis or assembly, or the final polynucleotide synthesis products may be sequenced as a quality control check to determine whether the desired polynucleotide or polynucleotides have been correctly synthesised or assembled. The polynucleotide or polynucleotides of interest can be removed from the solid phase synthesis platform and sequenced by any one of a number of known commercially available sequencing techniques such as nanopore sequencing using a MinION™ device sold by Oxford Nanopore Technologies Ltd. In a particular example, the sequencing may be carried out on the solid phase platform itself, removing the need to transfer the polynucleotide to a separate synthesis device. Sequencing may be conveniently carried out on the same electrowetting device, such as an EWOD device as used for synthesis whereby the synthesis device comprises one or more measurement electrode pairs. A droplet comprising the polynucleotide of interest can be contacted with one of the electrodes of the electrode pair, the droplet forming a droplet interface bilayer with a second droplet in contact with the second electrode of the electrode pair wherein the droplet bilayer interface comprises a nanopore in an amphipathic membrane. The polynucleotide can be caused to translocate the nanopore for example under enzyme control and ion current flow through the nanopore can be measured under a potential difference between the electrode pair during passage of the polynucleotide through the nanopore. The ion current measurements over time can be recorded and used to determine the polynucleotide sequence. Prior to sequencing, the polynucleotide may be subjected to one or more sample preparation steps in order to optimise it for sequencing such as disclosed in patent application no. PCT/GB2015/050140. Examples of enzymes, amphipathic membranes and nanopores which may be suitably employed are disclosed in patent application nos. PCT/GB2013/052767 and PCT/GB2014/052736. The necessary reagents for sample preparation of the polynucleotide, nanopores, amphipathic membranes and so on may be supplied to the EWOD device via sample inlet ports. The sample inlet ports may be connected to reagent chambers.

### Surface Attachment Chemistries

Although oligonucleotides will typically be attached chemically, they may also be attached to surfaces by indirect means such as via affinity interactions. For example, oligonucleotides may be functionalised with biotin and bound to surfaces coated with avidin or streptavidin.

For the immobilization of polynucleotides to surfaces (e.g. planar surfaces), microparticles and beads etc., a variety of surface attachment methods and chemistries are available. Surfaces may be functionalised or derivatized to facilitate attachment. Such functionalisations are known in the art. For example, a surface may be functionalised with a polyhistidine-tag (hexa histidine-tag, 6×His-tag, His6 tag or His-Tag®), Ni-NTA, streptavidin, biotin, an oligonucleotide, a polynucleotide (such as DNA, RNA, PNA, GNA, TNA or LNA), carboxyl groups, quaternary amine groups, thiol groups, azide groups, alkyne groups, DIBO, lipid, FLAG-tag (FLAG octapeptide), polynucleotide binding proteins, peptides, proteins, antibodies or antibody fragments. The surface may be functionalised with a molecule or group which specifically binds to the anchor/scaffold polynucleotide.

Some examples of chemistries suitable for attaching polynucleotides to surfaces are shown in FIG. 12i and FIG. 12j.

In any of the methods described herein the scaffold polynucleotide comprising the synthesis strand comprising the primer strand portion and the portion of the support strand hybridized thereto may be tethered to a common surface via one or more covalent bonds. The one or more covalent bonds may be formed between a functional group on the common surface and a functional group on the scaffold molecule. The functional group on the scaffold molecule may be e.g. an amine group, a thiol group, a thiophosphate group or a thioamide group. The functional group on the common surface may be a bromoacetyl group, optionally wherein the bromoacetyl group is provided on a polyacrylamide surface derived using N-(5-bromoacetamidylpentyl) acrylamide (BRAPA).

In any of the methods of the invention a scaffold polynucleotide may be attached to a surface, either directly or indirectly, via a linker. Any suitable linker which is biocompatible and hydrophilic in nature may be used.

A linker may be a linear linker or a branched linker.

A linker may comprise a hydrocarbon chain. A hydrocarbon chain may comprise from 2 to about 2000 or more carbon atoms. The hydrocarbon chain may comprise an alkylene group, e.g. C2 to about 2000 or more alkylene groups. The hydrocarbon chain may have a general formula of —(CH₂)ₙ— wherein n is from 2 to about 2000 or more. The hydrocarbon chain may be optionally interrupted by one or more ester groups (i.e. —C(O)—O—) or one or more amide groups (i.e. —C(O)—N(H)—).

Any linker may be used selected from the group comprising PEG, polyacrylamide, poly(2-hydroxyethyl methacrylate), Poly-2-methyl-2-oxazoline (PMOXA), zwitterionic polymers, e.g. poly(carboxybetaine methacrylate) (PCBMA), poly[N-(3-sulfopropyl)-N-methacryloxyethyl-N, N dimethyl ammonium betaine] (PSBMA), glycopolymers, and polypeptides.

A linker may comprise a polyethylene glycol (PEG) having a general formula of —(CH₂—CH₂—O)ₙ—, wherein n is from 1 to about 600 or more.

A linker may comprise oligoethylene glycol-phosphate units having a general formula of —[(CH₂—CH₂—O)ₙ—PO₂—O]ₘ— where n is from 1 to about 600 or more and m could be 1-200 or more.

Any of the above-described linkers may be attached at one end of the linker to a scaffold molecule as described herein, and at the other end of the linker to a first functional group wherein the first functional group may provide a covalent attachment to a surface.

The first functional group may be e.g. an amine group, a thiol group, a thiophosphate group or a thioamide group as further described herein. The surface may be functionalised with a further functional group to provide a covalent bond with the first functional group. The further functional group may be e.g. a 2-bromoacetamido group as further described herein. Optionally a bromoacetyl group is provided on a polyacrylamide surface derived using N-(5-bromoacetamidylpentyl) acrylamide (BRAPA). The further functional group on the surface may be a bromoacetyl group, optionally wherein the bromoacetyl group is provided on a polyacrylamide surface derived using N-(5-bromoacetamidylpentyl) acrylamide (BRAPA) and the first functional group may be e.g. an amine group, a thiol group, a thiophosphate group or a thioamide group as appropriate. The surface to which polynucleotides are attached may comprise a gel. The surface comprises a polyacrylamide surface, such as about 2% polyacrylamide, preferably the polyacrylamide surface is coupled to a solid support such as glass.

In any of the methods of the invention a scaffold polynucleotide may optionally be attached to a linker via a branching nucleotide incorporated into the scaffold polynucleotide. Any suitable branching nucleotide may be used with any suitable compatible linker.

Prior to initiating synthesis cycles of the invention, scaffold polynucleotides may be synthesised with one or more branching nucleotides incorporated into the scaffold polynucleotide. The exact position at which the one or more branching nucleotides are incorporated into the scaffold polynucleotide, and thus where a linker may be attached, may vary and may be chosen as desired. The position may e.g. be at the terminal end of a support strand and/or a synthesis strand or e.g. in the loop region which connects the support strand to the synthesis strand in embodiments which comprise a hairpin loop.

During synthesis of the scaffold polynucleotide the one or more branching nucleotides may be incorporated into the scaffold polynucleotide with a blocking group which blocks a reactive group of the branching moiety. The blocking group may then be removed (deblocked) prior to the coupling to the branching moiety of the linker, or a first unit (molecule) of the linker if a linker comprises multiple units.

During synthesis of the scaffold polynucleotide the one or more branching nucleotides may be incorporated into the scaffold polynucleotide with a group suitable for use in a subsequent “click chemistry” reaction to couple to the branching moiety the linker, or a first unit of the linker if a linker comprises multiple units. An example of such a group is an acetylene group.

Some non-limiting exemplary branching nucleotides are shown below.

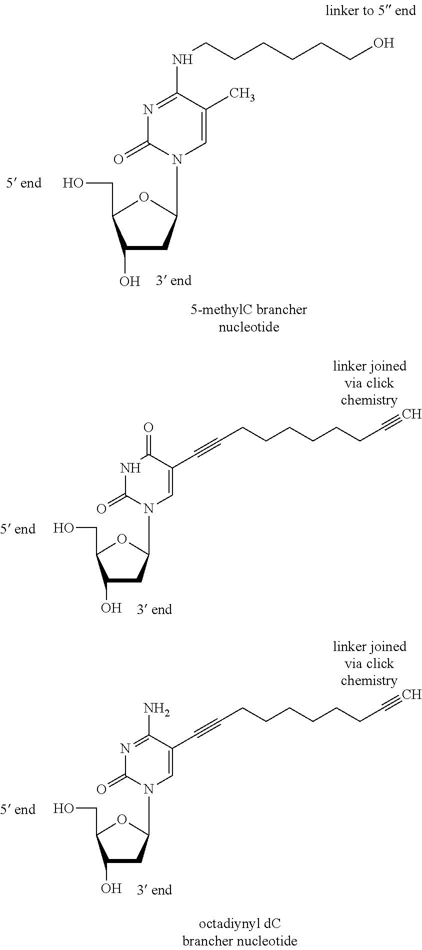 [SMILES: Cc1cn([C@H]2C[C@@H](O)[C@@H](CO)O2)c(=O)nc1NCCCCCCO ; C#CCCCCCCC#Cc1cn([C@H]2C[C@@H](O)[C@@H](CO)O2)c(=O)[nH]c1=O ; C#CCCCCCCC#Cc1cn([C@H]2C[C@@H](O)[C@@H](CO)O2)c(=O)nc1N]

A linker may optionally comprise one or more spacer molecules (units), such as e.g. an Sp9 spacer, wherein the first spacer unit is attached to the branching nucleotide.

The linker may comprise one or more further spacer groups attached to the first spacer group. For example, the linker may comprise multiple e.g. Sp9 spacer groups. A first spacer group is attached to the branching moiety and then one or more further spacer groups are sequentially added to extend a spacer chain comprising multiple spacer units connected with phosphate groups therebetween.

Shown below are some non-limiting examples of spacer units (Sp3, Sp9 and Sp13) which could comprise the first spacer unit attached to a branching nucleotide, or a further spacer unit to be attached to an existing spacer unit already attached to the branching nucleotide.

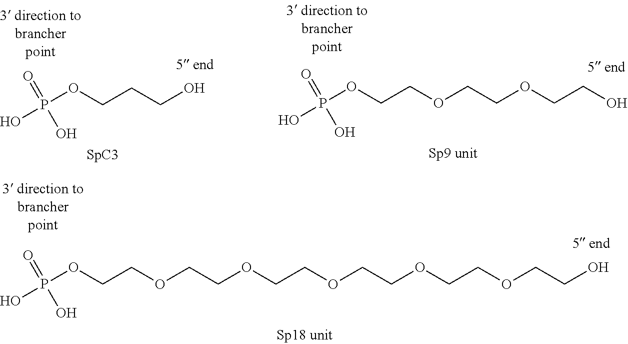 [SMILES: O=P(O)(O)OCCCO ; O=P(O)(O)OCCOCCOCCO ; O=P(O)(O)OCCOCCOCCOCCOCCOCCO]

A linker may comprise one or more ethylene glycol units.

A linker may comprise an oligonucleotide, wherein multiple units are nucleotides.

In the structures depicted above the term 5″ is used to differentiate from the 5′ end of the nucleotide to which the branching moiety is attached, wherein 5′ has its ordinary meaning in the art. By 5″ it is intended to mean a position on the nucleotide from which a linker can be extended. The 5″ position may vary. The 5″ position is typically a position in the nucleobase of the nucleotide. The 5″ position in the nucleobase may vary depending on the nature of the desired branching moiety, as depicted in the structures above.

### Microarrays

Any of the polynucleotide synthesis methods described herein may be used to manufacture a polynucleotide microarray (Trevino, V. et al., Mol. Med. 2007 13, pp527-541). Thus anchor or scaffold polynucleotides may be tethered to a plurality of individually addressable reaction sites on a surface and polynucleotides having a predefined sequence may be synthesised in situ on the microarray.

Following synthesis, at each reaction area the polynucleotide of predefined sequence may be provided with a unique sequence. The anchor or scaffold polynucleotides may be provided with barcode sequences to facilitate identification.

Other than the method of synthesising the polynucleotides of predefined sequence, microarray manufacture may be performed using techniques commonly used in this technical field, including techniques described herein. For example, anchor or scaffold polynucleotides may be tethered to surfaces using known surface attachment methods and chemistries, including those described herein.

Following synthesis of the polynucleotides of predefined sequence, there may be provided a final cleavage step to remove any unwanted polynucleotide sequence from untethered ends.

Polynucleotides of predefined sequence may be provided at reaction sites in double-stranded form. Alternatively, following synthesis double-stranded polynucleotides may be separated and one strand removed, leaving single-stranded polynucleotides at reaction sites. Selective tethering of strands may be provided to facilitate this process. For example, in methods involving a scaffold polynucleotide the synthesis strand may be tethered to a surface and the support strand may be untethered, or vice versa. The synthesis strand may be provided with a non-cleavable linker and the support strand may be provided with a cleavable linker, or vice versa. Separation of strands may be performed by conventional methods, such as heat treatment.

### Assembly of Synthetic Polynucleotides

A polynucleotide having a predefined sequence synthesised by methods described herein, and optionally amplified by methods described herein, may be joined to one or more other such polynucleotides to create larger synthetic polynucleotides.

Joining of multiple polynucleotides can be achieved by techniques commonly known in the art. A first polynucleotide and one or more additional polynucleotides synthesised by methods described herein may be cleaved to create compatible termini and then polynucleotides joined together by ligation. Cleavage can be achieved by any suitable means. Typically, restriction enzyme cleavage sites may be created in polynucleotides and then restriction enzymes used to perform the cleavage step, thus releasing the synthesised polynucleotides from any anchor/scaffold polynucleotide. Cleavage sites could be designed as part of the anchor/scaffold polynucleotides. Alternatively, cleavage sites could be created within the newly-synthesised polynucleotide as part of the predefined nucleotide sequence.

Assembly of polynucleotides is preferably performed using solid phase methods. For example, following synthesis a first polynucleotide may be subject to a single cleavage at a suitable position distal to the site of surface immobilization. The first polynucleotide will thus remain immobilized to the surface, and the single cleavage will generate a terminus compatible for joining to another polynucleotide. An additional polynucleotide may be subject to cleavage at two suitable positions to generate at each terminus a compatible end for joining to other polynucleotides, and at the same time releasing the additional polynucleotide from surface immobilization. The additional polynucleotide may be compatibly joined with the first polynucleotide thus creating a larger immobilized polynucleotide having a predefined sequence and having a terminus compatible for joining to yet another additional polynucleotide. Thus iterative cycles of joining of preselected cleaved synthetic polynucleotides may create much longer synthetic polynucleotide molecules. The order of joining of the additional polynucleotides will be determined by the required predefined sequence.

Thus the assembly methods of the invention may allow the creation of synthetic polynucleotide molecules having lengths in the order of one or more Mb.

The assembly and/or synthesis methods of the invention may be performed using apparatuses known in the art. Techniques and apparatuses are available which allow very small volumes of reagents to be selectively moved, partitioned and combined with other volumes in different locations of an array, typically in the form of droplets Electrowetting techniques, such as electrowetting-on-dielectric (EWOD), may be employed, as described above. Suitable electrowetting techniques and systems that may be employed in the invention that are able to manipulate droplets are disclosed for example in U.S. Pat. Nos. 8,653,832, 8,828,336, US20140197028 and US20140202863.

Cleavage from the solid phase may be achieved by providing cleavable linkers in one or both the primer strand portion and the portion of the support strand hybridized thereto. The cleavable linker may be e.g. a UV cleavable linker.

Examples of cleavage methods involving enzymatic cleavage are shown in FIG. 30. The schematic shows a scaffold polynucleotide attached to a surface (via black diamond structures) and comprising a polynucleotide of predefined sequence. The scaffold polynucleotide comprises top and bottom hairpins. In each case the top hairpin can be cleaved using a cleavage step utilizing the universal nucleotide to define a cleavage site. The bottom hairpin can be removed by a restriction endonuclease via a site that is engineered into the scaffold polynucleotide or engineered into the newly-synthesised polynucleotide of predefined sequence.

Thus polynucleotides having a predefined sequence may be synthesised whilst immobilized to an electrowetting surface, as described above. Synthesised polynucleotides may be cleaved from the electrowetting surface and moved under the influence of an electric field in the form of a droplet. Droplets may be combined at specific reaction sites on the surface where they may deliver cleaved synthesised polynucleotides for ligation with other cleaved synthesised polynucleotides. Polynucleotides can then be joined, for example by ligation. Using such techniques populations of different polynucleotides may be synthesised and attached in order according to the predefined sequence desired. Using such systems a fully automated polynucleotide synthesis and assembly system may be designed. The system may be programmed to receive a desired sequence, supply reagents, perform synthesis cycles and subsequently assemble desired polynucleotides according to the predefined sequence desired.

### Systems and Kits

The invention also provides polynucleotide synthesis systems for carrying out any of the synthesis methods described and defined herein, as well as any of the subsequent amplification and assembly steps described and defined herein.

Typically, synthesis cycle reactions will be carried out by incorporating nucleotides of predefined sequence into scaffold polynucleotide molecules which are tethered to a surface by means described and defined herein. The surface may be any suitable surface as described and defined herein.

In one embodiment, reactions to incorporate nucleotides of predefined sequence into a scaffold polynucleotide molecule involve performing any of the synthesis methods on a scaffold polynucleotide within a reaction area.

A reaction area is any area of a suitable substrate to which a scaffold polynucleotide molecule is attached and wherein reagents for performing the synthesis methods may be delivered.

In one embodiment a reaction area may be a single area of a surface comprising a single scaffold polynucleotide molecule wherein the single scaffold polynucleotide molecule can be addressed with reagents.

In another embodiment a reaction area may be a single area of a surface comprising multiple scaffold polynucleotide molecules, wherein the scaffold polynucleotide molecules cannot be individually addressed with reagent in isolation from each other. Thus in such an embodiment the multiple scaffold polynucleotide molecules in the reaction area are exposed to the same reagents and conditions and may thus give rise to synthetic polynucleotide molecules having the same or substantially the same nucleotide sequence.

In one embodiment a synthesis system for carrying out any of the synthesis methods described and defined herein may comprise multiple reaction areas, wherein each reaction area comprises one or more attached scaffold polynucleotide molecules and wherein each reaction area may be individually addressed with reagent in isolation from each of the other reaction areas. Such a system may be configured e.g. in the form of an array, e.g. wherein reaction areas are formed upon a substrate, typically a planar substrate.

A system having a substrate comprising a single reaction area or comprising multiple reaction areas may be comprised within e.g. an EWOD system or a microfluidic system and the systems configured to deliver reagents to the reaction site. EWOD and microfluidic systems are described in more detail herein. For example an EWOD system may be configured to deliver reagents to the reaction site(s) under electrical control. A microfluidic system, such as comprising microfabricated architecture e.g. as formed from elastomeric or similar material, may be configured to deliver reagents to the reaction site(s) under fluidic pressure and/or suction control or by mechanical means. Reagents may be delivered by any suitable means, for example via carbon nanotubes acting as conduits for reagent delivery. Any suitable system may be envisaged.

EWOD, microfluidic and other systems may be configured to deliver any other desired reagents to reaction sites, such as enzymes for cleaving a synthesised double-stranded polynucleotide from the scaffold polynucleotide following synthesis, and/or reagents for cleaving a linker to release an entire scaffold polynucleotide from the substrate and/or reagents for amplifying a polynucleotide molecule following synthesis or any region or portion thereof, and/or reagents for assembling larger polynucleotide molecules from smaller polynucleotide molecules which have been synthesised according to the synthesis methods of the invention.

The invention also provides kits for carrying out any of the synthesis methods described and defined herein. A kit may contain any desired combination of reagents for performing any of the synthesis and/or assembly methods of the invention described and defined herein. For example, a kit may comprise any one or more volume(s) of reaction reagents comprising scaffold polynucleotides, volume(s) of reaction reagents corresponding to any one or more steps of the synthesis cycles described and defined herein, volume(s) of reaction reagents comprising nucleotides comprising reversible blocking groups or reversible terminator groups, volume(s) of reaction reagents for amplifying one or more polynucleotide molecules following synthesis or any region or portion thereof, volume(s) of reaction reagents for assembling larger polynucleotide molecules from smaller polynucleotide molecules which have been synthesised according to the synthesis methods of the invention, volume(s) of reaction reagents for cleaving a synthesised double-stranded polynucleotide from the scaffold polynucleotide following synthesis, and volume(s) of reaction reagents for cleaving one or more linkers to release entire scaffold polynucleotides from a substrate.

### Data Storage

Polynucleotide molecules are naturally capable of storing information encoded within them due to differences in the identity and sequences of nucleobases forming the structure of the polynucleotide molecule. The natural data storage function of polynucleotide molecules can be exploited for the storage of new information by synthesising new polynucleotide molecules according to a specific nucleobase sequence which can thus encode new information within the polynucleotide molecule which can later be accessed or “read” to retrieve the information.

New information can, for example, be encoded into a polynucleotide molecule in a digital form. Thus the invention additionally provides methods of storing data in digital form in a polynucleotide molecule, thereby generating a nucleotide sequence in the polynucleotide synthesis molecule indicative of the “0” or “1” state of a bit of digital information.

A nucleotide sequence can be incorporated into a polynucleotide synthesis molecule to be indicative of the “0” or “1” state of a bit of digital information in any suitable way. For example bits of digital information can be created using two different species of nucleotide. For example a scaffold polynucleotide can be extended so as to generate an adenine (A)—thymine (T) pair in a first cycle of synthesis followed by extension so as to generate a cytosine (C)—guanine (G) pair in a second subsequent cycle. The presence of the A-T pair in the scaffold polynucleotide molecule can thus be indicative of the “0” or “1” state of a bit of digital information. The presence of the C-G pair juxtaposed adjacent to the A-T pair can thus be indicative of the opposite state of the bit. Incorporation of multiple A-T and C-G pairs of nucleobases in sequence can therefore allow for digital information to be encoded into the scaffold polynucleotide in bit form. A-T and C-G are provided as examples only. Any nucleobases can be used provided they can be distinguished from each other.

Incorporation of single nucleobases of alternating species is one way of generating a bit of digital information. Bits can alternatively be generated by the incorporation of two or more, i.e. a first string, of nucleobases of the same or indistinguishable species in the same or successive cycles of synthesis which can thus be indicative of the “0” or “1” state of a bit of digital information. This can then be followed by the incorporation of two or more, i.e. a second string, of nucleobases of the same or indistinguishable species in the same or successive cycles of synthesis which can thus be indicative of the opposite state of the bit to that previously generated. Any nucleobases can be used provided that the nucleobases of the first string can be distinguished from the nucleobases of the second string. First and second strings need not consist of the same number of nucleobases since the transition between first and second string is indicative of the transition between the “0” or “1” state of the bit of digital information and the opposite state of the bit.

Any of the extended scaffold polynucleotides described and defined herein may be followed by a step of determining the sequence of the extended scaffold polynucleotide. Such a step may be carried out using a nanopore using nanopore sequencing techniques which are well known in the art. By way of further example, a step of determining the sequence of the extended scaffold polynucleotide may be carried out subsequent to a method of storing data in a polynucleotide molecule, such as described herein, for example to provide a write-read system.

Any such method of data storage may be performed using any of the in vitro methods of synthesising a double-stranded polynucleotide molecule as described and defined herein. Any such method of data storage may be performed using any of the apparatus, devices and systems described and defined herein.

### Exemplary Methods

Exemplary non-limiting methods of synthesising a polynucleotide or an oligonucleotide molecule according to the invention are described herein, including in the appended claims.

In the following six exemplary methods, and variants thereof, of synthesising a polynucleotide or an oligonucleotide molecule according to the invention, references to synthesis method versions of the invention 1 to 10 are to be interpreted according to the reaction schematics set out respectively in FIGS. 1 to 10, and not according to the reaction schematics set out in any of FIGS. 11 to 15, 57, 60 and 61 or descriptions of the same in the Examples section. The reaction schematics set out in any of FIGS. 11 to 15, 57, 60 and 61 and descriptions of the same in the Examples section below provide illustrative support for the methods of the invention based on ligation-mediated reaction schemes which are modified in comparison with the methods of the present invention.

In each exemplary method described below, the structures described in each step may be referred to by reference to specific Figures with the aid of reference signs as appropriate. The reference signs in the text below correspond with those in FIGS. 1 to 10. Such references signs are not intended to be limited to the specific structures shown in the Figures, and the descriptions of the relevant structures correspond to the descriptions thereof as provided herein in its entirety, including but not limited to those specifically illustrated.

In each of FIGS. 1 to 10 the following structures are depicted.

A double-stranded scaffold polynucleotide molecule is shown in step (1) (provision of scaffold) having a left strand and a right strand hybridized thereto. In the methods described herein a scaffold polynucleotide is described as having a “first” strand and a “second” strand. The first strand is designated as such because it is the first strand to be extended in a given cycle of synthesis. The second strand is designated as such because it is the second strand to be extended in a given cycle of synthesis. In each of FIGS. 1 to 10 a first strand is depicted with dotted lines and a second strand is depicted with dashed-and-dotted lines. In step (1) (provision of scaffold) of FIGS. 1 to 4, 7 and 8, the first strand is depicted as the right hand strand, and the second strand is depicted as the left hand strand. In step (1) (provision of scaffold) of FIGS. 5 6, 9 and 10 the first strand is depicted as the left hand strand and the second strand is depicted as the right hand strand.

In each of methods 1 to 10 the first and second strands of the scaffold polynucleotide are extended at the same end. Thus in the case of FIGS. 1 to 10, the ends of the scaffold polynucleotide to be extended are the upper ends. The lower ends of the scaffold polynucleotide (i.e. which are labelled 3′ and 5′) are not shown to be extended.

In the case of FIGS. 1 to 10, a filled circle, such as attached to the “A” nucleotide at the 5′ terminal end of the left strand shown at step (1) (provision of scaffold) of FIG. 1, represents a 5′ phosphate group or any other suitable ligatable 5′ group as part of the nucleotide.

Steps (2) and (4) depict first and second extension/ligation steps, wherein a polynucleotide ligation molecule is ligated to the scaffold polynucleotide. A polynucleotide ligation molecule is depicted as a double-stranded structure having a synthesis strand depicted with a solid line, and a helper strand depicted with a dashed line, and which is hybridised to the synthesis strand. The synthesis strand comprises a universal nucleotide depicted as “Un”. A non-ligatable nucleotide positioned at the 3′ terminal end of a helper strand is depicted with a filled star-shaped structure, such as depicted at the 3′ terminal end of the helper strand of the polynucleotide ligation molecule shown in step (2) of FIGS. 1 to 4. A non-ligatable nucleotide positioned at the 5′ terminal end of a helper strand is depicted with a filled diamond-shaped structure, such as depicted at the 5′ terminal end of the helper strand of the polynucleotide ligation molecule shown in step (2) of FIGS. 5 and 6.

A single-stranded break in a ligated scaffold polynucleotide is depicted with two horizontal lines and labelled “nick”.

Cleavage is depicted via a jagged arrowhead, such as shown in steps (3) and (5) of FIGS. 1 to 10.

Ten non-limiting exemplary methods of the invention, referred to herein as synthesis method versions of the invention 1 to 10 respectively, are described below (see FIGS. 1 to 10 respectively). Each method comprises five principle steps. In step (1) a scaffold polynucleotide is provided. Step (2) comprises a first extension/ligation reaction wherein a first polynucleotide ligation molecule is ligated to the scaffold polynucleotide and the first strand of the scaffold polynucleotide is extended with one or more nucleotides derived from the first polynucleotide ligation molecule. Step (3) comprises a first cleavage reaction wherein the ligated scaffold polynucleotide is cleaved at a cleavage site leading to loss of the first polynucleotide ligation molecule and retention, in the first strand of the scaffold polynucleotide, of the one or more nucleotides derived from the first polynucleotide ligation molecule. Step (4) comprises a second extension/ligation reaction wherein a second polynucleotide ligation molecule is ligated to the scaffold polynucleotide and the second strand of the scaffold polynucleotide is extended with one or more nucleotides derived from the second polynucleotide ligation molecule. Step (5) comprises a second cleavage reaction wherein the ligated scaffold polynucleotide is cleaved at a cleavage site leading to loss of the second polynucleotide ligation molecule and retention, in the second strand of the scaffold polynucleotide, of the one or more nucleotides derived from the second polynucleotide ligation molecule.

Provision of Scaffold Step (1)

With reference to FIGS. 1 to 10 describing ten specific non-limiting exemplary versions of the synthesis methods of the invention, a double-stranded scaffold polynucleotide is initially provided (step 1; 101, 102, 103 etc). The double-stranded scaffold polynucleotide comprises a first strand and a second strand hybridised thereto.

The scaffold polynucleotide is double-stranded and provides a support structure to accommodate the region of synthetic polynucleotide as it is synthesised de novo. In each of FIGS. 1 to 10 the scaffold polynucleotide comprises a first strand depicted with a dotted line and a second strand depicted with a dashed-and-dotted line.

The double-stranded scaffold polynucleotide has one end which is to be extended, shown in the Figures as the upper end. The lower end of the scaffold polynucleotide is shown in the Figures labelled 3′ and 5 and is not shown to be extended.

In step (1) of each of methods 1 to 4, 7 and 8 (FIGS. 1 to 4, 7 and 8 respectively), the terminal nucleotide of the second strand at the end of the scaffold polynucleotide to be extended comprises a phosphate group or any other suitable ligatable group, and therefore this terminal nucleotide is a ligatable nucleotide. In methods 1 to 4, the terminal nucleotide of the first strand at the end of the scaffold polynucleotide to be extended comprises a hydroxyl group or any other suitable ligatable group, and therefore this terminal nucleotide is also a ligatable nucleotide. In methods 5 6, 9 and 10 the terminal nucleotide of the first strand at the end of the scaffold polynucleotide to be extended comprises a phosphate group or any other suitable ligatable group, and therefore this terminal nucleotide is a ligatable nucleotide. In methods 5 6, 9 and 10 the terminal nucleotide of the second strand at the end of the scaffold polynucleotide to be extended comprises a hydroxyl group or any other suitable ligatable group, and therefore this terminal nucleotide is also a ligatable nucleotide.

First Extension/Ligation Step (1)

In step (2) of the methods a first extension/ligation step is performed (102, 202, 302, etc.) wherein a first polynucleotide ligation molecule is ligated to the double-stranded scaffold polynucleotide. The first polynucleotide ligation molecule comprises one or more nucleotides of the predefined nucleotide sequence. In the case of method versions 1, 2 and 7 to 10 the first polynucleotide ligation molecule comprises two nucleotides of the predefined nucleotide sequence. In the case of method versions 3 to 6 the first polynucleotide ligation molecule comprises one nucleotide of the predefined nucleotide sequence.

The first polynucleotide ligation molecule comprises a synthesis strand (solid line) and a helper strand (dashed line) hybridized to the synthesis strand.

The first polynucleotide ligation molecule of the first ligation reaction may comprise a sticky-ended complementary ligation end, i.e. with a single overhanging nucleotide, such as in method versions 1 and 2. In these versions, the terminal nucleotide of the synthesis strand overhangs the terminal nucleotide of the helper strand. Alternatively, the polynucleotide ligation molecule of the first ligation reaction may comprise a blunt-ended complementary ligation end, i.e. with no overhanging nucleotides, such as in method versions 3 to 6.

The complementary ligation end is complementary with the end of the double-stranded scaffold polynucleotide to be extended. The synthesis strand of the first polynucleotide ligation molecule comprises one or more nucleotides of the predefined nucleotide sequence at the complementary ligation end. The first nucleotide of the predefined nucleotide sequence is the terminal nucleotide of the synthesis strand of the first polynucleotide ligation molecule at the complementary ligation end. The first nucleotide of the predefined nucleotide sequence is a ligatable nucleotide and is ligated to the terminal nucleotide of the first strand of the scaffold polynucleotide. Upon ligation, the first nucleotide of the predefined nucleotide sequence of that cycle is incorporated, together with any additional nucleotides of the predefined nucleotide sequence, into the double-stranded scaffold polynucleotide by ligation of the terminal nucleotide of the synthesis strand of the first polynucleotide ligation molecule to the terminal nucleotide of the first strand of the double-stranded scaffold polynucleotide. In method versions 3 to 6 the first strand of the scaffold polynucleotide is extended by only a single nucleotide, and therefore the polynucleotide ligation molecule carries only one nucleotide of the predefined sequence and comprises a blunt-ended complementary ligation end. A corresponding blunt end is provided in the scaffold polynucleotide at the end of the scaffold polynucleotide to be extended. In contrast, in method versions 1 and 2, the first strand of the scaffold polynucleotide is extended by two nucleotides, the polynucleotide ligation molecule consequently carries two nucleotides of the predefined sequence and comprises a complementary ligation end having a single-base overhang, with the terminal nucleotide of the synthesis strand overhanging the terminal nucleotide of the helper strand. A corresponding single-base overhang is provided in the scaffold polynucleotide, with the terminal nucleotide of the second strand overhanging the terminal nucleotide of the first strand at the end of the scaffold polynucleotide to be extended.

In all six method versions, and variants thereof, the synthesis strand of the first polynucleotide ligation molecule comprises a universal nucleotide (labelled “Un” in the structures depicted in step (2) of each of FIGS. 1 to 10) at the complementary ligation end which will facilitate cleavage in the first cleavage step (3). The role of the universal nucleotide will be apparent from the detailed description of each method below.

The terminal nucleotide of the helper strand of the first polynucleotide ligation molecule is provided such that at the complementary ligation end the helper strand cannot be ligated to the corresponding strand of the scaffold polynucleotide (typically the second strand), i.e. it is provided with a non-ligatable terminal nucleotide. If the terminal nucleotide of the helper strand is at the 3′ end of the helper strand, the nucleotide may be provided as a non-ligatable 2′,3′-dideoxynucleotide or a 2′-deoxynucleotide, or any other suitable non-ligatable nucleotide. If the terminal nucleotide of the helper strand is at the 5′ end of the helper strand, the nucleotide may be provided without a phosphate group, i.e. it may be provided as a nucleoside. Alternatively a 5′-protected nucleoside, a nucleoside with a non-ligatable group at the 5′ position, such as 5′-deoxynucleoside or a 5′-aminonucleoside, or any other suitable non-ligatable nucleotide or nucleoside may be used.

Thus upon ligation of the synthesis strand of the first polynucleotide ligation molecule to the first strand of the double-stranded scaffold polynucleotide, a single-strand break or “nick” is provided between the terminal nucleotide of the helper strand of the first polynucleotide ligation molecule and the terminal nucleotide of the second strand of the scaffold polynucleotide.

In the first ligation step (2), if the first nucleotide of the synthesis strand of the polynucleotide ligation molecule, which is to be ligated to the terminal nucleotide of the first strand of the double-stranded scaffold polynucleotide, is at the 5′ end of the synthesis strand of the polynucleotide ligation molecule, it must be provided, prior to the ligation step, with an attached phosphate group or other ligatable group so as to allow the terminal nucleotide of the synthesis strand of the polynucleotide ligation molecule to act as a substrate for the ligase enzyme. Similarly, the 3′ end of the first strand of the double-stranded scaffold polynucleotide must be provided, prior to the ligation step, with a hydroxyl group, or other ligatable group, so as to allow the terminal nucleotide of the first strand of the double-stranded scaffold polynucleotide to act as a substrate for the ligase enzyme. Vice versa, if the first nucleotide of the synthesis strand of the polynucleotide ligation molecule, which is to be ligated to the terminal nucleotide of the first strand of the double-stranded scaffold polynucleotide, is at the 3′ end of the synthesis strand of the polynucleotide ligation molecule, it must be provided, prior to the ligation step, with a hydroxyl group or other ligatable group so as to allow the terminal nucleotide of the synthesis strand of the polynucleotide ligation molecule to act as a substrate for the ligase enzyme. Similarly, the 5′ end of the first strand of the double-stranded scaffold polynucleotide must be provided, prior to the ligation step, with an attached phosphate group, or other ligatable group, so as to allow the terminal nucleotide of the first strand of the double-stranded scaffold polynucleotide to act as a substrate for the ligase enzyme.

Upon ligation of the first polynucleotide ligation molecule to the double-stranded scaffold polynucleotide a double-stranded scaffold polynucleotide is formed comprising the newly incorporated one or more nucleotides, a universal nucleotide for use in facilitating cleavage in the first cleavage step (3) and a “nick” in the second strand of the scaffold polynucleotide.

First Cleavage Step (3)

Following the step of incorporating the one or more nucleotides into the first strand following the first ligation step (2), the scaffold polynucleotide is then subjected to a first cleavage step (step 3, 103, 203, 303, etc).

In method versions 1 to 5, 7, 8 and 9 the first cleavage step comprises cleaving the first strand of the ligated scaffold polynucleotide immediately after the universal nucleotide in the direction distal to the synthesis strand of the ligated first polynucleotide ligation molecule, i.e. the support strand is cleaved between the position occupied by the universal nucleotide and the next nucleotide position in the first strand in the direction distal to the synthesis strand of the ligated first polynucleotide ligation molecule. In method versions 6 and 10 cleavage comprises cleaving the first strand of the ligated scaffold polynucleotide between the nucleotides occupying the first and second positions after the universal nucleotide in the direction distal to the synthesis strand of the ligated first polynucleotide ligation molecule.

Cleavage results in release of the polynucleotide ligation molecule from the scaffold polynucleotide and retention of the one or more nucleotides attached to the first strand of the cleaved scaffold polynucleotide. Cleavage results in release of the helper strand of the first polynucleotide ligation molecule which is hybridised to the synthesis strand immediately prior to cleavage, and release of the synthesis strand comprising the universal nucleotide. Cleavage thus leaves in place a cleaved double-stranded scaffold polynucleotide comprising, at the site of cleavage, a cleaved terminal end of the first strand and the terminal end of the second strand which comprised the nick site prior to cleavage. The cleaved double-stranded scaffold polynucleotide comprises the one or more nucleotides of the predefined sequence derived from the first polynucleotide ligation molecule as the terminal nucleotide(s) of the cleaved end of the first strand of the scaffold polynucleotide.

In each of exemplary methods of the invention 1 to 6, the first cleavage step (3) leaves in place a single-base overhang at the cleaved end of the scaffold polynucleotide, i.e. at the end of the scaffold polynucleotide which is extended in the first extension/ligation step (2). In each of exemplary methods of the invention 7 to 10, the first cleavage step (3) leaves in place a double-base overhang at the cleaved end of the scaffold polynucleotide. In each method the terminal or nucleotide or the terminal and penultimate nucleotides of the first strand of the cleaved scaffold polynucleotide at the cleaved end overhangs the terminal nucleotide of the second strand of the cleaved scaffold polynucleotide at the cleaved end. In methods 1 to 4, 7 and 8, the terminal nucleotide(s) at the 3′ end of the first strand overhangs the terminal nucleotide at the 5′ of the second strand. In methods 5, 6, 9 and 10 the terminal nucleotide(s) at the 5′ end of the first strand overhangs the terminal nucleotide at the 3′ end of the second strand. The single-base overhang generated in the first cleavage step provides a complementary end for the complementary ligation end of the second polynucleotide ligation molecule of the second ligation step (4) of the same cycle of synthesis.

Second Extension/Ligation Step (4)

In step (4) of the methods a second extension/ligation step is performed (104, 204, 304, etc.) wherein a second polynucleotide ligation molecule is ligated to the cleaved double-stranded scaffold polynucleotide. The second polynucleotide ligation molecule comprises one or more nucleotides of the predefined nucleotide sequence. In the case of method versions 1, 2 and 7 to 10 the second polynucleotide ligation molecule comprises two nucleotides of the predefined nucleotide sequence. In the case of method versions 3 to 6 the second polynucleotide ligation molecule comprises one nucleotide of the predefined nucleotide sequence.

The second polynucleotide ligation molecule comprises a synthesis strand (solid line) and a helper strand (dashed line) hybridized to the synthesis strand.

The polynucleotide ligation molecule of the second ligation reaction comprises a sticky-ended complementary ligation end, i.e. with a single overhanging nucleotide, wherein the terminal nucleotide of the synthesis strand overhangs the terminal nucleotide of the helper strand of the second polynucleotide ligation molecule.

The complementary ligation end is complementary with the end of the cleaved double-stranded scaffold polynucleotide to be extended. The synthesis strand of the second polynucleotide ligation molecule comprises one or more nucleotides of the predefined nucleotide sequence at the terminal end of the complementary ligation end. The terminal nucleotide of the synthesis strand of the second polynucleotide ligation molecule is a ligatable nucleotide and is ligated to the terminal nucleotide of the second strand of the cleaved scaffold polynucleotide. Upon ligation, the terminal nucleotide of the synthesis strand of the polynucleotide ligation molecule is incorporated, together with any additional nucleotides of the predefined nucleotide sequence, into the cleaved double-stranded scaffold polynucleotide by ligation of the terminal nucleotide of the synthesis strand of the second polynucleotide ligation molecule to the terminal nucleotide of the second strand of the cleaved double-stranded scaffold polynucleotide.

In method versions 3 to 6 the second strand of the scaffold polynucleotide is extended by only a single nucleotide, and therefore the polynucleotide ligation molecule carries only one nucleotide of the predefined sequence. In contrast, in method versions 1, 2 and 7 to 10 the second strand of the scaffold polynucleotide is extended by two nucleotides, the polynucleotide ligation molecule consequently carries two nucleotides of the predefined sequence.

In all six method versions, and variants thereof, the synthesis strand of the second polynucleotide ligation molecule also comprises a universal nucleotide (labelled “Un” in the structures depicted in step (4) of each of FIGS. 1 to 10) at the complementary ligation end which will facilitate cleavage in the second cleavage step (5). The role of the universal nucleotide will be apparent from the detailed description of each method below.

The terminal nucleotide of the helper strand of the second polynucleotide ligation molecule is provided such that at the complementary ligation end the helper strand cannot be ligated to the corresponding strand of the scaffold polynucleotide (typically the first strand), i.e. it is provided with a non-ligatable terminal nucleotide. If the terminal nucleotide of the helper strand is at the 3′ end of the helper strand, the nucleotide may be provided as a non-ligatable 2′,3′-dideoxynucleotide, a 2′-deoxynucleotide or any other suitable non-ligatable nucleotide. If the terminal nucleotide of the helper strand is at the 5′ end of the helper strand, the nucleotide may be provided without a phosphate group, i.e. it may be provided as a nucleoside. Alternatively a 5′-protected nucleoside, a nucleoside with a non-ligatable group at the 5′ position, such as 5′-deoxynucleoside or a 5′-aminonucleoside, or any other suitable non-ligatable nucleotide or nucleoside may be used.

Thus upon ligation of the synthesis strand of the second polynucleotide ligation molecule to the second strand of the double-stranded scaffold polynucleotide, a single-strand break or “nick” is provided in between the terminal nucleotide of the helper strand of the second polynucleotide ligation molecule and the terminal nucleotide of the first strand of the scaffold polynucleotide.

Analogous to the first ligation step (2), in the second ligation step (4) if the first nucleotide of the synthesis strand of the second polynucleotide ligation molecule, which is to be ligated to the terminal nucleotide of the second strand of the cleaved double-stranded scaffold polynucleotide, is at the 5′ end of the synthesis strand of the polynucleotide ligation molecule, it must be provided, prior to the ligation step, with an attached phosphate group or other ligatable group so as to allow the terminal nucleotide of the synthesis strand of the second polynucleotide ligation molecule to act as a substrate for the ligase enzyme. Similarly, the 3′ end of the second strand of the cleaved double-stranded scaffold polynucleotide must be provided, prior to the ligation step, with a hydroxyl group, or other ligatable group, so as to allow the terminal nucleotide of the second strand of the cleaved double-stranded scaffold polynucleotide to act as a substrate for the ligase enzyme. Vice versa, if the first nucleotide of the synthesis strand of the second polynucleotide ligation molecule, which is to be ligated to the terminal nucleotide of the second strand of the double-stranded scaffold polynucleotide, is at the 3′ end of the synthesis strand of the second polynucleotide ligation molecule, it must be provided, prior to the ligation step, with a hydroxyl group or other ligatable group so as to allow the terminal nucleotide of the synthesis strand of the second polynucleotide ligation molecule to act as a substrate for the ligase enzyme. Similarly, the 5′ end of the second strand of the cleaved double-stranded scaffold polynucleotide must be provided, prior to the ligation step, with an attached phosphate group, or other ligatable group, so as to allow the terminal nucleotide of the second strand of the cleaved double-stranded scaffold polynucleotide to act as a substrate for the ligase enzyme.

Upon ligation of the second polynucleotide ligation molecule to the double-stranded scaffold polynucleotide a double-stranded scaffold polynucleotide is formed comprising the newly incorporated one or more nucleotides in the second strand, a universal nucleotide for use in facilitating cleavage in the second cleavage step (5) and a “nick” in the first strand of the scaffold polynucleotide.

Second Cleavage Step (5)

Following the step of incorporating the one or more nucleotides into the second strand following the second ligation step (4), the scaffold polynucleotide is then subjected to a second cleavage step (step 5, 105, 205, 305, etc).

In method versions 1, 3, 5, 6, 7, 9 and 10 the second cleavage step comprises cleaving the second strand of the ligated scaffold polynucleotide immediately after the universal nucleotide in the direction distal to the synthesis strand of the ligated second polynucleotide ligation molecule, i.e. the second strand is cleaved between the position occupied by the universal nucleotide and the next nucleotide position in the second strand in the direction distal to the synthesis strand of the ligated second polynucleotide ligation molecule. In method versions 2, 4 and 8 cleavage comprises cleaving the second strand of the ligated scaffold polynucleotide between the nucleotides occupying the first and second positions after the universal nucleotide in the direction distal to the synthesis strand of the ligated second polynucleotide ligation molecule.

Cleavage results in release of the polynucleotide ligation molecule from the scaffold polynucleotide and retention of the one or more nucleotides attached to the second strand of the cleaved scaffold polynucleotide. Cleavage results in release of the helper strand of the second polynucleotide ligation molecule which is hybridised to the synthesis strand immediately prior to cleavage, and release of the synthesis strand comprising the universal nucleotide. Cleavage thus leaves in place a cleaved double-stranded scaffold polynucleotide comprising, at the site of cleavage, a cleaved terminal end of the second strand and the terminal end of the first strand which comprised the nick site prior to cleavage, and wherein the cleaved double-stranded scaffold polynucleotide comprises the one or more nucleotides of the predefined sequence derived from the second polynucleotide ligation molecule as the terminal nucleotide(s) of the cleaved end of the second strand of the scaffold polynucleotide.

In each of exemplary methods of the invention 1 and 2, the second cleavage step (5) leaves in place a single-base overhang at the cleaved end of the scaffold polynucleotide, i.e. at the end of the scaffold polynucleotide which is extended in the second extension/ligation step (4). In each of methods 1 and 2 the terminal nucleotide of the second strand of the cleaved scaffold polynucleotide at the cleaved end overhangs the terminal nucleotide of the first strand of the cleaved scaffold polynucleotide at the cleaved end. In methods 1 and 2, the terminal nucleotide at the 5′ end of the second strand overhangs the terminal nucleotide at the 3′ of the first strand. The single-base overhang generated in the second cleavage step provides a complementary end for the overhanging complementary ligation end of the first polynucleotide ligation molecule of the first ligation step (2) of the next cycle of synthesis.

In each of exemplary methods of the invention 3 to 10, the second cleavage step (5) leaves in place a blunt end at the cleaved end of the scaffold polynucleotide, i.e. at the end of the scaffold polynucleotide which is extended in the second extension/ligation step (4), with no overhanging nucleotides. The blunt end of the cleaved scaffold polynucleotide generated in the second cleavage step (5) provides a complementary end for the blunt-ended complementary ligation end of the first polynucleotide ligation molecule of the first ligation step (2) of the next cycle of synthesis.

The cleaved double-stranded scaffold polynucleotide arising from the second cleavage step (5) acts as a double-stranded scaffold polynucleotide for a first ligation reaction (2) in the next cycle of synthesis.

At the end of a cycle of synthesis one or more nucleotides are incorporated into the first strand of the double-stranded scaffold polynucleotide and one or more nucleotides are incorporated into the second strand of the double-stranded scaffold polynucleotide. In method versions 3 to 6 one nucleotide is incorporated into each of the first and second strands thereby forming a nucleotide pair. In method versions 1 and 2 two nucleotides are incorporated into each of the first and second strands, wherein one nucleotide in each strand forms a nucleotide pair, one nucleotide incorporated into the first strand forms a nucleotide pair with the terminal nucleotide of the second strand of the scaffold polynucleotide initially provided in step (1), and one nucleotide incorporated into the second strand remains unpaired and forms the terminal single-base overhang at the cleaved end of the scaffold polynucleotide. In method versions 7 to 10 two nucleotides are incorporated into each of the first and second strands thereby forming two nucleotide pairs.

In the context of the first cycles of synthesis as described in any of the methods herein, the terms “first nucleotide of the predefined sequence” and “second nucleotide of the predefined sequence”, or similar terms, in relation to extension of the first and second strands are not necessarily to be understood as meaning the very first or second nucleotide of the predefined sequence. The methods described herein relate to the synthesis of a double-stranded polynucleotide having a predefined sequence and a portion of the predefined sequence may be provided pre-synthesised in the scaffold polynucleotide before initiation of the first cycle of synthesis. In this context the term “a” first nucleotide of the predefined sequence can mean “any” nucleotide of the predefined sequence. Thus in this context the terms “first” and “second” nucleotides of the predefined sequence” may be considered merely “a” further nucleotide of the predefined sequence. In the case of the specific and non-limiting method versions 1 to 10 of the invention defined herein, and certain variants thereof, each “first nucleotide” ligated to the scaffold polynucleotide in a given cycle will be sequentially ligated to the incorporated nucleotide(s) of the previous cycle in the same nucleic acid strand, thereby extending the first or second strand sequentially by one or more further nucleotides per cycle. Thus, when synthesis cycles are completed the synthesised double-stranded polynucleotide molecule will comprise a predefined sequence of one strand defined by the ligated first and second (if present) nucleotides of each cycle, and a predefined sequence of the opposite strand defined by the incorporated first and second (if present) nucleotides of each cycle.

All exemplary methods of the invention may be performed such that the first strand is synthesised to comprise a specific desired predefined sequence selected by the user. In such methods the sequence of the second strand may not necessarily comprise a specific desired predefined sequence. Vice versa, all exemplary methods of the invention may be performed such that the second strand is synthesised to comprise a specific desired predefined sequence selected by the user. In such methods the sequence of the first strand may not necessarily comprise a specific desired predefined sequence. Since the methods of the invention provide for the synthesis of a double-stranded polynucleotide molecule, the user may, if desired, separate the two strands at the end of the desired number of cycles of synthesis, discard one of the strands, retain the other strand and copy the other strand to form a double-stranded polynucleotide molecule wherein the strand which is copied from the said other strand comprises a sequence which is complementary to the sequence of the said other strand. In such a situation, because one of the strands of the double-stranded polynucleotide molecule synthesised by the methods of the invention is discarded, its polynucleotide sequence is not critical and does not for example need to be complementary to the sequence of the said other strand which is retained for copying. Thus it may be possible, for example, to structure the strand to be discarded with nucleotides which are not perfectly complementary with nucleotides of the predefined sequence selected by the user to be incorporated into the said other strand which is retained for copying. For example, it may be possible to incorporate one or more universal nucleotides into the strand to be discarded which will pair with nucleotides of the predefined sequence selected by the user to be incorporated into the said other strand which is retained for copying. In such a method, the strand to be discarded simply functions as a support strand for the said other strand which is retained for copying. In this context the invention provides in vitro methods of synthesising a double-stranded polynucleotide wherein at least one strand, i.e. the said other strand which is retained for copying, has a predefined sequence. The sequence of the strand to be discarded may be random, or semi-random. Thus in any of the methods of the invention described or defined herein, the term “predefined” as it relates to extension of the first strand or the second strand must be interpreted accordingly.

In what follows below, the first six method versions of the invention will be explained in detail with reference to FIGS. 1 to 6 respectively. Method versions of the invention 7 to 10 can be considered to be variants of other methods and will be described in that context as exemplary variant methods.

### Synthesis Method Version 1

### Step 1—Provision of a Scaffold Polynucleotide

In exemplary version 1 of the synthesis methods of the invention a double-stranded scaffold polynucleotide is provided in step (1) (101). The double-stranded scaffold polynucleotide is provided comprising a first strand and a second strand hybridised thereto. The terminal nucleotide of the first strand at the end to be extended is positioned at the 3′ end of the first strand and comprises a hydroxyl group or any other suitable 3′ ligatable group and therefore this terminal nucleotide is a ligatable nucleotide. In FIG. 1 this nucleotide is depicted as “X” and can be any nucleotide, nucleotide analogue or nucleotide derivative. The terminal nucleotide of the first strand at the end to be extended is depicted as paired with the penultimate nucleotide of the 5′ end of the second strand. This penultimate nucleotide is depicted as “X” and can be any nucleotide, nucleotide analogue or nucleotide derivative, and may or may not be complementary to its partner nucleotide in the pair. Preferably it is complementary. The terminal nucleotide of the 5′ end of the second strand is unpaired and forms a single-base overhang and overhangs the terminal 3′ nucleotide of the first strand. The terminal nucleotide of the 5′ end of the second strand is depicted as “A”. However this nucleotide can be any nucleotide, nucleotide analogue or nucleotide derivative. This nucleotide can be considered to be a nucleotide of the prefined sequence. The terminal nucleotide of the 5′ end of the second strand comprises a phosphate group or any other suitable 5′ ligatable group, and therefore this terminal nucleotide is also a ligatable nucleotide.

The terminal ends of the scaffold polynucleotide which are not shown to be extended, i.e. those labelled 3′ and 5′ in FIG. 1, are preferably attached to a substrate, such as by any of the means described herein.

### Step 2—Ligation of a First Polynucleotide Ligation Molecule to the Scaffold Polynucleotide and Incorporation of One or More Nucleotides of the Predefined Sequence

In step (2) of the method a double-stranded polynucleotide ligation molecule is ligated (102) to the scaffold polynucleotide in a sticky-(complementary)-ended ligation reaction by the action of an enzyme having ligase activity.

The polynucleotide ligation molecule comprises a synthesis strand and a helper strand hybridised thereto. The polynucleotide ligation molecule further comprises a complementary ligation end comprising in the synthesis strand a universal nucleotide and two nucleotides of the predefined sequence.

The complementary ligation end of the first polynucleotide ligation molecule is structured such that the terminal and penultimate nucleotides of the synthesis strand are respectively the first and second nucleotides of the predefined sequence to be incorporated into the first strand of the scaffold polynucleotide in any given cycle of synthesis.

The complementary ligation end comprises a single-base overhang. The terminal nucleotide of the synthesis strand is unpaired and overhangs the terminal nucleotide of the helper strand. The penultimate nucleotide of the synthesis strand is paired with the terminal nucleotide of the helper strand. In FIG. 1 the terminal nucleotide of the synthesis strand is depicted as “T”, the penultimate nucleotide of the synthesis strand is depicted as “G”, and the terminal nucleotide of the helper strand is depicted as “C”. Each of these designations are for illustrative purposes only. They can be any nucleotide, nucleotide analogue or nucleotide derivative. The penultimate nucleotide of the synthesis strand and the terminal nucleotide of the helper strand may or may not be complementary. Preferably they are complementary.

At the terminal end of the synthesis strand at the complementary ligation end of the first polynucleotide ligation molecule the universal nucleotide occupies the position immediately next to the penultimate nucleotide in the direction distal to the complementary ligation end. The universal nucleotide forms a nucleotide pair with the nucleotide which is the penultimate nucleotide in the helper strand in the direction distal to the complementary ligation end. This is depicted in FIG. 1 as “X”. It can be any nucleotide, nucleotide analogue or nucleotide derivative.

The terminal nucleotide of the synthesis strand at the complementary ligation end of the first polynucleotide ligation molecule is depicted in FIG. 1 at the 5′ end of the synthesis strand. This nucleotide is provided as a ligatable nucleotide and comprises a phosphate group or any other suitable 5′ ligatable group. The terminal nucleotide of the helper strand at the complementary ligation end of the first polynucleotide ligation molecule is depicted in FIG. 1 at the 3′ end of the helper strand. This nucleotide is provided as a non-ligatable nucleotide and comprises a non-ligatable 2′,3′-dideoxynucleotide or a 2′-deoxynucleotide, or any other suitable non-ligatable 3′ nucleotide.

The terminal nucleotide of the synthesis strand, i.e. the first nucleotide of the predefined sequence to be incorporated into the first strand in a given cycle of synthesis, occupies nucleotide position n in the synthesis strand. By position n in the synthesis strand of the first polynucleotide ligation molecule it is meant the position which will be occupied by the first nucleotide which is to be attached to the terminal end of the first strand of the scaffold polynucleotide following ligation of the polynucleotide ligation molecule to the scaffold polynucleotide. Position n also refers to the nucleotide position in the first strand of the ligated scaffold polynucleotide which is occupied by the said first nucleotide following its attachment to the terminal end of the first strand after ligation. Position n also refers to the nucleotide position in the second strand of the scaffold polynucleotide which will be occupied by a partner nucleotide for the said first nucleotide following attachment of the partner nucleotide to the terminal end of the second strand after the second extension/ligation reaction. Position n also refers to the nucleotide position which is occupied by the said partner nucleotide following its attachment to the terminal end of the second strand after the second extension/ligation reaction. The universal nucleotide in the synthesis strand of the first polynucleotide ligation molecule occupies position n+2.

The complementary ligation end of the first polynucleotide ligation molecule is configured so that it will compatibly join with the overhanging end of the scaffold polynucleotide when subjected to suitable ligation conditions. Upon ligation of the synthesis strand of the polynucleotide ligation molecule and the first strand of the scaffold polynucleotide, the terminal and penultimate nucleotides of the synthesis strand become incorporated into the first strand of the scaffold polynucleotide. Because the terminal nucleotide of the helper strand of the first polynucleotide ligation molecule is a non-ligatable nucleotide, the ligase enzyme will be prevented from ligating the helper strand of the first polynucleotide ligation molecule and the second strand of the scaffold polynucleotide, thus creating a single-strand break or “nick” between the helper strand of the first polynucleotide ligation molecule and the second strand of the scaffold polynucleotide.

Ligation of the polynucleotide ligation molecule to the scaffold polynucleotide extends the length of the first strand of the double-stranded scaffold polynucleotide of step (1) and wherein the terminal and penultimate nucleotides of the synthesis strand of the first polynucleotide ligation molecule are incorporated into the first strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

### Step 3—First Cleavage Step

In step (3) of the method the ligated scaffold polynucleotide is cleaved (103) at a cleavage site. The cleavage site is defined by a sequence comprising the universal nucleotide in the synthesis strand of the ligated first polynucleotide ligation molecule. Cleavage results in a double-strand break in the scaffold polynucleotide. Cleavage of the scaffold polynucleotide (step 3) results in loss of the helper strand of the ligated first polynucleotide ligation molecule and loss of the synthesis strand comprising the universal nucleotide. Cleavage of the scaffold polynucleotide thereby releases the polynucleotide ligation molecule from the scaffold polynucleotide but leads to the retention of the first and second nucleotides of that cycle attached to the first strand of the cleaved scaffold polynucleotide. Cleavage of the scaffold polynucleotide leaves in place a cleaved double-stranded scaffold polynucleotide comprising a singe-base overhang at the cleaved end. The second nucleotide of the predefined sequence occupies a position (n+1) as the terminal nucleotide of the first strand of the cleaved double-stranded scaffold polynucleotide, and the first nucleotide of the predefined sequence occupies a position (n) as the penultimate nucleotide of the cleaved first strand. The first nucleotide of the predefined sequence is paired with the nucleotide which was the terminal overhanging nucleotide of the second strand in the double-stranded scaffold polynucleotide in step (1) (101) prior to the first extension/ligation step. In FIG. 1 these nucleotides are depicted (103) as “A” and “T” for illustrative purposes only. Each one of these nucleotides can be any nucleotide, nucleotide analogue or nucleotide derivative and the pair may or may not be complementary. Preferably they are complementary.

The second strand of the ligated scaffold polynucleotide is already provided with a single-strand break or “nick” in this exemplary method, thus only cleavage of the first strand is necessary to provide a double-strand break in the scaffold polynucleotide. Furthermore, as noted previously, in this exemplary method version cleavage generates a cleaved double-stranded scaffold polynucleotide with a single-base overhang, with the terminal nucleotide of the first strand overhanging the terminal nucleotide of the second strand, and the universal nucleotide occupies position n+2 in the first strand prior to the cleavage step. To obtain such a cleaved double-stranded scaffold polynucleotide with a singe-base overhang when the universal nucleotide occupies position n+2 in the first strand, the first ligated strand is cleaved at a specific position relative to the universal nucleotide. When the first strand of the scaffold polynucleotide is cleaved between nucleotide positions n+2 and n+1 the polynucleotide ligation molecule is released from the scaffold polynucleotide (see the structure depicted as exiting the synthesis cycle immediately after cleavage step 3 (103) in FIG. 1) except that the first and second nucleotides of that cycle derived from the polynucleotide ligation molecule in step (2) are retained in the scaffold polynucleotide attached to the first strand of the cleaved scaffold polynucleotide.

A phosphate group, or any other suitable 5′ ligatable group, should continue to be attached to the terminal nucleotide of the second strand of the cleaved scaffold polynucleotide at the cleavage site. This ensures that the second strand of the cleaved scaffold polynucleotide can be ligated to the synthesis strand of the second polynucleotide ligation molecule in the second extension/ligation step (4). Cleavage is performed so that the terminal nucleotide of the first strand of the cleaved scaffold polynucleotide retains a ligatable group, typically a hydroxyl group or any other suitable 3′ ligatable group, at the 3′ end of the first strand.

Thus in method version 1 the universal nucleotide occupies position n+2 in the synthesis/first strand at step (2) and the first strand is cleaved between nucleotide positions n+2 and n+1 at step (3).

Preferably, the second strand is cleaved by cleavage of the phosphodiester bond between nucleotide positions n+2 and n+1 (the first phosphodiester bond of the first strand relative to the position of the universal nucleotide, in the direction distal to the ligated polynucleotide ligation molecule/proximal to the first strand).

The first strand may be cleaved by cleavage of one ester bond of the phosphodiester bond between nucleotide positions n+2 and n+1.

Preferably the first strand is cleaved by cleavage of the first ester bond relative to nucleotide position n+2.

Any suitable mechanism may be employed to effect cleavage of the first strand between nucleotide positions n+2 and n+1 when the universal nucleotide occupies position n+2.

Cleavage of the first strand between nucleotide positions n+2 and n+1 as described above may be performed by the action of an enzyme.

Cleavage of the first strand between nucleotide positions n+2 and n+1 as described above may be performed as a two-step cleavage process.

The first cleavage step of a two-step cleavage process may comprise removing the universal nucleotide from the first strand thus forming an abasic site at position n+2, and the second cleavage step may comprise cleaving the first strand at the abasic site, between positions n+2 and n+1.

One mechanism of cleaving the first strand at a cleavage site defined by a sequence comprising a universal nucleotide in the manner outlined above is described in analogous fashion in Example 2. The cleavage mechanism described in Example 2 is exemplary and other mechanisms could be employed, provided that the cleaved double-stranded scaffold polynucleotide described above is achieved.

In the first cleavage step of a two-step cleavage process the universal nucleotide is removed from the first strand whilst leaving the sugar-phosphate backbone intact. This can be achieved by the action of an enzyme which can specifically excise a single universal nucleotide from a double-stranded polynucleotide. In the exemplified cleavage methods the universal nucleotide is inosine and inosine is excised from the first strand by the action of an enzyme, thus forming an abasic site. In the exemplified cleavage method the enzyme is a 3-methyladenine DNA glycosylase enzyme, specifically human alkyladenine DNA glycosylase (hAAG). Other enzymes, molecules or chemicals could be used provided that an abasic site is formed. The nucleotide-excising enzyme may be an enzyme which catalyses the release of uracil from polynucleotides, such as Uracil-DNA Glycosylase (UDG).

In the second step of a two-step cleavage process the first strand is cleaved at the abasic site by making a single-strand break. In the exemplified methods the first strand is cleaved by the action of a chemical which is a base, such as NaOH. Alternatively, an organic chemical such as N,N′-dimethylethylenediamine may be used. Alternatively, enzymes having abasic site lyase activity, such as AP Endonuclease 1, Endonuclease III (Nth), or Endonuclease VIII, may be used. These enzymes cleave the DNA backbone at a phosphate group which is positioned 5′ relative to the abasic site. Cleavage thus exposes an OH group at the 3′ terminal end of the first strand which provides a terminal 3′ nucleotide which is ligatable in the first ligation step in the next cycle. Other enzymes, molecules or chemicals could be used provided that the first strand is cleaved at the abasic site as described.

Thus in embodiments wherein the universal nucleotide is at position n+2 of the first strand at step (2) and the first strand is cleaved between positions n+2 and n+1, a first cleavage step may be performed with a nucleotide-excising enzyme. An example of such an enzyme is a 3-methyladenine DNA glycosylase enzyme, such as human alkyladenine DNA glycosylase (hAAG). The second cleavage step may be performed with a chemical which is a base, such as NaOH. The second step may be performed with an organic chemical having abasic site cleavage activity such as N,N′-dimethylethylenediamine. The second step may be performed with an enzyme having abasic site lyase activity such as Endonuclease VIII or Endonuclease III.

Cleavage of the first strand between nucleotide positions n+2 and n+1 as described above may also be performed as a one-step cleavage process. Examples of enzymes which may be used in any such process include Endonuclease III, Endonuclease VIII. Other enzymes which may be used in any such process include enzymes which cleave 8-oxoguanosine, such as formamidopyrimidine DNA glycosylase (Fpg) and 8-oxoguanine DNA glycosylase (hOGG1), which cleaves the DNA backbone so as to leave a phosphate group at the 3′ terminal end of the cleaved first strand, which can then be removed by Endonuclease IV or T4 polynucleotide kinase to expose an OH group which is ligatable in the first ligation step in the next cycle.

### Step 4—Ligation of a Second Polynucleotide Ligation Molecule to the Scaffold Polynucleotide and Incorporation of One or More Further Nucleotides of the Predefined Sequence

In step (4) of the method a second double-stranded polynucleotide ligation molecule is ligated (104) to the scaffold polynucleotide in a sticky—(complementary)—ended ligation reaction by the action of an enzyme having ligase activity.

The second polynucleotide ligation molecule comprises a synthesis strand and a helper strand hybridised thereto. The second polynucleotide ligation molecule further comprises a complementary ligation end comprising in the synthesis strand a universal nucleotide and two further nucleotides of the predefined sequence.

The complementary ligation end of the second polynucleotide ligation molecule is structured such that the terminal and penultimate nucleotides of the synthesis strand are respectively the first and second nucleotides of the predefined sequence to be incorporated into the second strand of the scaffold polynucleotide in any given cycle of synthesis.

The complementary ligation end comprises a single-base overhang. The terminal nucleotide of the synthesis strand is unpaired and overhangs the terminal nucleotide of the helper strand. The penultimate nucleotide of the synthesis strand is paired with the terminal nucleotide of the helper strand. In FIG. 1 the terminal nucleotide of the synthesis strand is depicted as “C”, the penultimate nucleotide of the synthesis strand is depicted as “T”, and the terminal nucleotide of the helper strand is depicted as “A”. Each of these designations are for illustrative purposes only. They can be any nucleotide, nucleotide analogue or nucleotide derivative. The penultimate nucleotide of the synthesis strand and the terminal nucleotide of the helper strand may or may not be complementary. Preferably they are complementary.

At the terminal end of the synthesis strand at the complementary ligation end of the second polynucleotide ligation molecule the universal nucleotide occupies the position immediately next to the penultimate nucleotide in the direction distal to the complementary ligation end. The universal nucleotide forms a nucleotide pair with the nucleotide which is the penultimate nucleotide in the helper strand in the direction distal to the complementary ligation end. This is depicted in FIG. 1 as “X”. It can be any nucleotide, nucleotide analogue or nucleotide derivative.

The terminal nucleotide of the synthesis strand at the complementary ligation end of the second polynucleotide ligation molecule is depicted in FIG. 1 at the 3′ end of the synthesis strand. This nucleotide is provided as a ligatable nucleotide and comprises a hydroxyl group or any other suitable 3′ ligatable group. The terminal nucleotide of the helper strand at the complementary ligation end of the first polynucleotide ligation molecule is depicted in FIG. 1 at the 5′ end of the helper strand. This nucleotide is provided as a non-ligatable nucleotide, e.g. lacking a phosphate group or provided with any suitable 5′ blocking group which can prevent ligation.

The terminal nucleotide of the synthesis strand, i.e. the first nucleotide of the predefined sequence of that cycle which is added to the second strand of the cleaved scaffold polynucleotide, occupies nucleotide position n+1 in the synthesis strand. With reference to the definition of position n in the first extension/ligation reaction of step (2), position n refers to the nucleotide position in the second strand of the scaffold polynucleotide which is occupied by a partner nucleotide for the first nucleotide which is attached to the terminal end of the first strand after the first extension/ligation reaction of step (2). The universal nucleotide in the synthesis strand of the second polynucleotide ligation molecule occupies position n+3.

The complementary ligation end of the second polynucleotide ligation molecule is configured so that it will compatibly join with the overhanging end of the cleaved scaffold polynucleotide, generated in step (3), when subjected to suitable ligation conditions. Upon ligation of the synthesis strand of the second polynucleotide ligation molecule and the second strand of the scaffold polynucleotide, the terminal and penultimate nucleotides of the synthesis strand of the second polynucleotide ligation become incorporated into the second strand of the cleaved scaffold polynucleotide. Because the terminal nucleotide of the helper strand of the second polynucleotide ligation molecule is a non-ligatable nucleotide, the ligase enzyme will be prevented from ligating the helper strand of the second polynucleotide ligation molecule and the first strand of the cleaved scaffold polynucleotide, thus creating a single-strand break or “nick” between the helper strand of the second polynucleotide ligation molecule and the first strand of the cleaved scaffold polynucleotide.

Ligation of the second polynucleotide ligation molecule to the cleaved scaffold polynucleotide extends the length of the second strand of the double-stranded scaffold polynucleotide of step (3) and wherein the terminal and penultimate nucleotides of the synthesis strand of the second polynucleotide ligation molecule are incorporated into the second strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

Upon ligation, the terminal nucleotide of the synthesis strand of the second polynucleotide ligation molecule pairs with the terminal nucleotide of the first strand of the cleaved scaffold polynucleotide generated in step (3), thus forming a nucleotide pair.

### Step 5—Second Cleavage Step

In step (5) of the method the ligated scaffold polynucleotide is cleaved (105) at a cleavage site. The cleavage site is defined by a sequence comprising the universal nucleotide in the synthesis strand of the ligated second polynucleotide ligation molecule. Cleavage results in a double-strand break in the scaffold polynucleotide. Cleavage of the scaffold polynucleotide (step 5) results in loss of the helper strand of the ligated second polynucleotide ligation molecule and loss of the synthesis strand comprising the universal nucleotide. Cleavage of the scaffold polynucleotide thereby releases the second polynucleotide ligation molecule from the scaffold polynucleotide but leads to the retention of the next two nucleotides of that cycle attached to the second strand of the cleaved scaffold polynucleotide. Cleavage of the scaffold polynucleotide leaves in place a cleaved double-stranded scaffold polynucleotide comprising a singe-base overhang at the cleaved end. The second nucleotide of the predefined sequence incorporated in step (4) occupies a position (n+2) as the terminal nucleotide of the second strand of the cleaved double-stranded scaffold polynucleotide, and the first nucleotide of the predefined sequence incorporated in step (4) occupies a position (n+1) as the penultimate nucleotide of the cleaved second strand. The first nucleotide of the predefined sequence incorporated in step (4) is paired with the nucleotide which was the terminal overhanging nucleotide of the first strand in the double-stranded scaffold polynucleotide in step (3) (103) following the first cleavage step. In FIG. 1 these nucleotides are depicted (105) as “C” and “G” for illustrative purposes only. Each one of these nucleotides can be any nucleotide, nucleotide analogue or nucleotide derivative and the pair may or may not be complementary. Preferably they are complementary.

Prior to the second cleavage step the first strand of the ligated scaffold polynucleotide is already provided with a single-strand break or “nick” in this exemplary method, thus only cleavage of the second strand is necessary to provide a double-strand break in the scaffold polynucleotide. Furthermore, as noted previously in this exemplary method version cleavage generates a cleaved double-stranded scaffold polynucleotide with a single-base overhang, with the terminal nucleotide of the second strand overhanging the terminal nucleotide of the first strand, and the universal nucleotide occupies position n+3 in the second strand prior to the cleavage step. To obtain such a cleaved double-stranded scaffold polynucleotide with a singe-base overhang when the universal nucleotide occupies position n+3 in the second strand, the second strand is cleaved at a specific position relative to the universal nucleotide. When the second strand of the scaffold polynucleotide is cleaved between nucleotide positions n+3 and n+2 the polynucleotide ligation molecule is released from the scaffold polynucleotide (see the structure depicted as exiting the synthesis cycle immediately after cleavage step 5 (105) in FIG. 1) except that the first and second nucleotides of that cycle derived from the second polynucleotide ligation molecule in step (4) are retained in the scaffold polynucleotide attached to the second strand of the cleaved scaffold polynucleotide.

A phosphate group, or any other suitable 5′ ligatable group, should continue to be attached to the terminal nucleotide of the second strand of the cleaved scaffold polynucleotide at the cleavage site. This ensures that the second strand of the cleaved scaffold polynucleotide can be ligated to the synthesis strand of the second polynucleotide ligation molecule in the second extension/ligation step (4) of the next cycle of synthesis. Cleavage is performed so that the terminal nucleotide of the first strand of the cleaved scaffold polynucleotide retains a ligatable group, typically a hydroxyl group or any other suitable 3′ ligatable group, at the 3′ end of the first strand.

Thus in method version 1 the universal nucleotide occupies position n+3 in the synthesis/second strand at step (4) and the second strand is cleaved between nucleotide positions n+3 and n+2 at step (5).

Preferably, the second strand is cleaved by cleavage of the phosphodiester bond between nucleotide positions n+3 and n+2 (the first phosphodiester bond of the second strand relative to the position of the universal nucleotide, in the direction distal to the ligated polynucleotide ligation molecule/proximal to the second strand).

The second strand may be cleaved by cleavage of one ester bond of the phosphodiester bond between nucleotide positions n+3 and n+2.

Preferably the second strand is cleaved by cleavage of the first ester bond relative to nucleotide position n+3.

Any suitable mechanism may be employed to effect cleavage of the second strand between nucleotide positions n+3 and n+2 when the universal nucleotide occupies position n+3.

Cleavage of the second strand between nucleotide positions n+3 and n+2 as described above may be performed by the action of an enzyme.

Cleavage of the second strand between nucleotide positions n+3 and n+2 as described above may be performed as a two-step cleavage process.

The first cleavage step of a two-step cleavage process may comprise removing the universal nucleotide from the second strand thus forming an abasic site at position n+3, and the second cleavage step may comprise cleaving the second strand at the abasic site, between positions n+3 and n+2.

One mechanism of cleaving the second strand at a cleavage site defined by a sequence comprising a universal nucleotide in the manner outlined above is described in analogous fashion in Example 2. The cleavage mechanism described in Example 2 is exemplary and other mechanisms could be employed, provided that the cleaved double-stranded scaffold polynucleotide described above is achieved.

In the first cleavage step of a two-step cleavage process the universal nucleotide is removed from the second strand whilst leaving the sugar-phosphate backbone intact. This can be achieved by the action of an enzyme which can specifically excise a single universal nucleotide from a double-stranded polynucleotide. In the exemplified cleavage methods the universal nucleotide is inosine and inosine is excised from the strand by the action of an enzyme, thus forming an abasic site. In the exemplified cleavage method the enzyme is a 3-methyladenine DNA glycosylase enzyme, specifically human alkyladenine DNA glycosylase (hAAG). Other enzymes, molecules or chemicals could be used provided that an abasic site is formed. The nucleotide-excising enzyme may be an enzyme which catalyses the release of uracil from polynucleotides, such as Uracil-DNA Glycosylase (UDG).

In the second step of a two-step cleavage process the second strand is cleaved at the abasic site by making a single-strand break. In the exemplified methods the strand is cleaved by the action of a chemical which is a base, such as NaOH. Alternatively, an organic chemical such as N,N′-dimethylethylenediamine may be used. Alternatively, enzymes having abasic site lyase activity, such as AP Endonuclease 1, Endonuclease III (Nth), or Endonuclease VIII, may be used. Other enzymes, molecules or chemicals could be used provided that the second strand is cleaved at the abasic site as described.

Thus in embodiments wherein the universal nucleotide is at position n+3 of the second strand at step (4) and the second strand is cleaved between positions n+3 and n+2, a first cleavage step may be performed with a nucleotide-excising enzyme. An example of such an enzyme is a 3-methyladenine DNA glycosylase enzyme, such as human alkyladenine DNA glycosylase (hAAG). The second cleavage step may be performed with a chemical which is a base, such as NaOH. The second step may be performed with an organic chemical having abasic site cleavage activity such as N,N′-dimethylethylenediamine. The second step may be performed with an enzyme having abasic site lyase activity such as Endonuclease VIII or Endonuclease III.

Cleavage of the second strand between nucleotide positions n+3 and n+2 as described above may also be performed as a one-step cleavage process. Examples of enzymes which may be used in any such process include Endonuclease III, Endonuclease VIII. Other enzymes which may be used in any such process include enzymes which cleave 8-oxoguanosine, such as formamidopyrimidine DNA glycosylase (Fpg) and 8-oxoguanine DNA glycosylase (hOGG1).

In synthesis method version 1 it will be noted that in any given cycle of synthesis, after the second cleavage step (step 5) the nucleotide position which is occupied by the terminal nucleotide of the second strand at the cleaved end is defined as nucleotide position n+2. This nucleotide position is defined as nucleotide position n in the next cycle of synthesis. Similarly, the nucleotide position which is occupied by the terminal nucleotide of the first strand at the cleaved end is defined as nucleotide position n+1. This nucleotide position is defined as nucleotide position n−1 in the next cycle of synthesis.

### Further Cycles

Following completion of the first cycle of synthesis, second and further cycles of synthesis may be performed using the same method steps.

The cleavage product of step (5) of the previous cycle is provided (in step 6) as the double-stranded scaffold polynucleotide for the next cycle of synthesis.

In step (7) of the next and each further cycle of synthesis a further first double-stranded polynucleotide ligation molecule is ligated to the cleavage product of step (5) of the previous cycle. The polynucleotide ligation molecule may be structured in the same way as described above for step (2) of the previous cycle, except that the further first polynucleotide ligation molecule comprises further first and second nucleotides of the further cycle of synthesis to be incorporated into the first strand. In step (7) the further first polynucleotide ligation molecule may be ligated to the cleavage product of step (5) of the previous cycle in the same way as described above for step (2).

In step (8) of the next and each further cycle of synthesis the ligated scaffold polynucleotide is subjected to a further first cleavage step at the cleavage site. Cleavage results in a double-strand break in the scaffold polynucleotide. Cleavage of the scaffold polynucleotide (step 8) results in loss of the helper strand and loss of the support strand comprising the universal nucleotide in the further first polynucleotide ligation molecule. Cleavage of the scaffold polynucleotide thereby releases the further first polynucleotide ligation molecule from the scaffold polynucleotide but leads to the retention of the further first and second nucleotides of that further cycle derived from the further first polynucleotide ligation molecule attached to the synthesis strand of the cleaved scaffold polynucleotide. Cleavage of the scaffold polynucleotide leaves in place a cleaved double-stranded scaffold polynucleotide comprising the further first and second nucleotides of the further cycle at the terminal end of the synthesis strand of the scaffold polynucleotide. Cleavage results in a single-base overhang with the terminal nucleotide of the first strand overhanging the terminal nucleotide of the second strand. Cleavage at step (8) may be performed in the same way as described above for step (4).

In step (9) of the next and each further cycle of synthesis a further second double-stranded polynucleotide ligation molecule is ligated to the cleavage product of step (8). The further second polynucleotide ligation molecule may be structured in the same way as described above for step (8) of the previous cycle, except that the further second polynucleotide ligation molecule comprises further first and second nucleotides of the further cycle of synthesis to be incorporated into the second strand. In step (9) the further second polynucleotide ligation molecule may be ligated to the cleavage product of step (8) in the same way as described above for step (4).

In step (10) of the next and each further cycle of synthesis the ligated scaffold polynucleotide is subjected to a further second cleavage step at the cleavage site. Cleavage results in a double-strand break in the scaffold polynucleotide. Cleavage of the scaffold polynucleotide (step 10) results in loss of the helper strand and loss of the support strand comprising the universal nucleotide in the further second polynucleotide ligation molecule. Cleavage of the scaffold polynucleotide thereby releases the further second polynucleotide ligation molecule from the scaffold polynucleotide but leads to the retention of the further first and second nucleotides of that further cycle derived from the further second polynucleotide ligation molecule attached to the synthesis strand of the cleaved scaffold polynucleotide. Cleavage of the scaffold polynucleotide leaves in place a cleaved double-stranded scaffold polynucleotide comprising the further first and second nucleotides of the further cycle derived from the further second polynucleotide ligation molecule at the terminal end of the second strand of the scaffold polynucleotide. The further second nucleotide is the terminal nucleotide of the second strand and overhangs the terminal nucleotide of the first strand in a single-base overhang. Cleavage at step (10) may be performed in the same way as described above for step (5).

### Synthesis Method Version 2

### Step 1—Provision of a Scaffold Polynucleotide

In exemplary version 2 of the synthesis methods of the invention a double-stranded scaffold polynucleotide is provided in step (1) (201). The double-stranded scaffold polynucleotide is provided comprising a first strand and a second strand hybridised thereto. The terminal nucleotide of the first strand at the end to be extended is positioned at the 3′ end of the first strand and comprises a hydroxyl group or any other suitable 3′ ligatable group and therefore this terminal nucleotide is a ligatable nucleotide. In FIG. 2 this nucleotide is depicted as “X” and can be any nucleotide, nucleotide analogue or nucleotide derivative. The terminal nucleotide of the first strand at the end to be extended is depicted as paired with the penultimate nucleotide of the 5′ end of the second strand. This penultimate nucleotide is depicted as “X” and can be any nucleotide, nucleotide analogue or nucleotide derivative, and may or may not be complementary to its partner nucleotide in the pair. Preferably it is complementary. The terminal nucleotide of the 5′ end of the second strand is unpaired and forms a single-base overhang and overhangs the terminal 3′ nucleotide of the first strand. The terminal nucleotide of the 5′ end of the second strand is depicted as “A”. However this nucleotide can be any nucleotide, nucleotide analogue or nucleotide derivative. This nucleotide can be considered to be a nucleotide of the predefined sequence. The terminal nucleotide of the 5′ end of the second strand comprises a phosphate group or any other suitable 5′ ligatable group, and therefore this terminal nucleotide is also a ligatable nucleotide.

The terminal ends of the scaffold polynucleotide which are not shown to be extended, i.e. those labelled 3′ and 5′ in FIG. 2, are preferably attached to a substrate, such as by any of the means described herein.

### Step 2—Ligation of a First Polynucleotide Ligation Molecule to the Scaffold Polynucleotide and Incorporation of One or More Nucleotides of the Predefined Sequence

In step (2) of the method a double-stranded polynucleotide ligation molecule is ligated (202) to the scaffold polynucleotide in a sticky—(complementary)—ended ligation reaction by the action of an enzyme having ligase activity.

The polynucleotide ligation molecule comprises a synthesis strand and a helper strand hybridised thereto. The polynucleotide ligation molecule further comprises a complementary ligation end comprising in the synthesis strand a universal nucleotide and two nucleotides of the predefined sequence.

The complementary ligation end of the first polynucleotide ligation molecule is structured such that the terminal and penultimate nucleotides of the synthesis strand are respectively the first and second nucleotides of the predefined sequence to be incorporated into the first strand of the scaffold polynucleotide in any given cycle of synthesis.

The complementary ligation end comprises a single-base overhang. The terminal nucleotide of the synthesis strand is unpaired and overhangs the terminal nucleotide of the helper strand. The penultimate nucleotide of the synthesis strand is paired with the terminal nucleotide of the helper strand. In FIG. 2 the terminal nucleotide of the synthesis strand is depicted as “T”, the penultimate nucleotide of the synthesis strand is depicted as “G”, and the terminal nucleotide of the helper strand is depicted as “C”. Each of these designations are for illustrative purposes only. They can be any nucleotide, nucleotide analogue or nucleotide derivative. The penultimate nucleotide of the synthesis strand and the terminal nucleotide of the helper strand may or may not be complementary. Preferably they are complementary.

At the terminal end of the synthesis strand at the complementary ligation end of the first polynucleotide ligation molecule the universal nucleotide occupies the position immediately next to the penultimate nucleotide in the direction distal to the complementary ligation end. The universal nucleotide forms a nucleotide pair with the nucleotide which is the penultimate nucleotide in the helper strand in the direction distal to the complementary ligation end.

The terminal nucleotide of the synthesis strand at the complementary ligation end of the first polynucleotide ligation molecule is depicted in FIG. 2 at the 5′ end of the synthesis strand. This nucleotide is provided as a ligatable nucleotide and comprises a phosphate group or any other suitable 5′ ligatable group. The terminal nucleotide of the helper strand at the complementary ligation end of the first polynucleotide ligation molecule is depicted in FIG. 2 at the 3′ end of the helper strand. This nucleotide is provided as a non-ligatable nucleotide and comprises a non-ligatable 2′,3′-dideoxynucleotide or a 2′-deoxynucleotide, or any other suitable non-ligatable 3′ nucleotide.

The terminal nucleotide of the synthesis strand, i.e. the first nucleotide of the predefined sequence to be incorporated into the first strand in a given cycle of synthesis, occupies nucleotide position n in the synthesis strand. By position n in the synthesis strand of the first polynucleotide ligation molecule it is meant the position which will be occupied by the first nucleotide which is to be attached to the terminal end of the first strand of the scaffold polynucleotide following ligation of the polynucleotide ligation molecule to the scaffold polynucleotide. Position n also refers to the nucleotide position in the first strand of the ligated scaffold polynucleotide which is occupied by the said first nucleotide following its attachment to the terminal end of the first strand after ligation. Position n also refers to the nucleotide position in the second strand of the scaffold polynucleotide which will be occupied by a partner nucleotide for the said first nucleotide following attachment of the partner nucleotide to the terminal end of the second strand after the second extension/ligation reaction. Position n also refers to the nucleotide position which is occupied by the said partner nucleotide following its attachment to the terminal end of the second strand after the second extension/ligation reaction. The universal nucleotide in the synthesis strand of the first polynucleotide ligation molecule occupies position n+2.

The complementary ligation end of the first polynucleotide ligation molecule is configured so that it will compatibly join with the overhanging end of the scaffold polynucleotide when subjected to suitable ligation conditions. Upon ligation of the synthesis strand of the polynucleotide ligation molecule and the first strand of the scaffold polynucleotide, the terminal and penultimate nucleotides of the synthesis strand become incorporated into the scaffold polynucleotide. Because the terminal nucleotide of the helper strand of the first polynucleotide ligation molecule is a non-ligatable nucleotide, the ligase enzyme will be prevented from ligating the helper strand of the first polynucleotide ligation molecule and the second strand of the scaffold polynucleotide, thus creating a single-strand break or “nick” between the helper strand of the first polynucleotide ligation molecule and the second strand of the scaffold polynucleotide.

Ligation of the polynucleotide ligation molecule to the scaffold polynucleotide extends the length of the first strand of the double-stranded scaffold polynucleotide of step (1) and wherein the terminal and penultimate nucleotides of the synthesis strand of the polynucleotide ligation molecule are incorporated into the first strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

### Step 3—First Cleavage Step

In step (3) of the method the ligated scaffold polynucleotide is cleaved (203) at a cleavage site. The cleavage site is defined by a sequence comprising the universal nucleotide in the synthesis strand of the ligated first polynucleotide ligation molecule. Cleavage results in a double-strand break in the scaffold polynucleotide. Cleavage of the scaffold polynucleotide (step 3) results in loss of the helper strand of the ligated first polynucleotide ligation molecule and loss of the synthesis strand comprising the universal nucleotide. Cleavage of the scaffold polynucleotide thereby releases the polynucleotide ligation molecule from the scaffold polynucleotide but leads to the retention of the first and second nucleotides of that cycle attached to the first strand of the cleaved scaffold polynucleotide. Cleavage of the scaffold polynucleotide leaves in place a cleaved double-stranded scaffold polynucleotide comprising a singe-base overhang at the cleaved end. The second nucleotide of the predefined sequence occupies a position (n+1) as the terminal nucleotide of the first strand of the cleaved double-stranded scaffold polynucleotide, and the first nucleotide of the predefined sequence occupies a position (n) as the penultimate nucleotide of the cleaved first strand. The first nucleotide of the predefined sequence is paired with the nucleotide which was the terminal overhanging nucleotide of the second strand in the double-stranded scaffold polynucleotide in step (1) (201) prior to the first extension/ligation step. In FIG. 2 these nucleotides are depicted (203) as “A” and “T” for illustrative purposes only. Each one of these nucleotides can be any nucleotide, nucleotide analogue or nucleotide derivative and the pair may or may not be complementary. Preferably they are complementary.

The second strand of the ligated scaffold polynucleotide is already provided with a single-strand break or “nick” in this exemplary method, thus only cleavage of the first strand is necessary to provide a double-strand break in the scaffold polynucleotide. Furthermore, as noted previously, in this exemplary method version cleavage generates a cleaved double-stranded scaffold polynucleotide with a single-base overhang, with the terminal nucleotide of the first strand overhanging the terminal nucleotide of the second strand, and the universal nucleotide occupies position n+2 in the first strand prior to the cleavage step. To obtain such a cleaved double-stranded scaffold polynucleotide with a singe-base overhang when the universal nucleotide occupies position n+2 in the first strand, the ligated first strand is cleaved at a specific position relative to the universal nucleotide. When the first strand of the scaffold polynucleotide is cleaved between nucleotide positions n+2 and n+1 the polynucleotide ligation molecule is released from the scaffold polynucleotide (see the structure depicted as exiting the synthesis cycle immediately after cleavage step 3 (203) in FIG. 2) except that the first and second nucleotides of that cycle derived from the first polynucleotide ligation molecule in step (2) are retained in the scaffold polynucleotide attached to the first strand of the cleaved scaffold polynucleotide.

A phosphate group, or any other suitable 5′ ligatable group, should continue to be attached to the terminal nucleotide of the second strand of the cleaved scaffold polynucleotide at the cleavage site. This ensures that the second strand of the cleaved scaffold polynucleotide can be ligated to the synthesis strand of the second polynucleotide ligation molecule in the second extension/ligation step (4). Cleavage is performed so that the terminal nucleotide of the first strand of the cleaved scaffold polynucleotide retains a ligatable group, typically a hydroxyl group or any other suitable 3′ ligatable group, at the 3′ end of the first strand.

Thus in method version 2 the universal nucleotide occupies position n+2 in the synthesis/first strand at step (2) and the first strand is cleaved between nucleotide positions n+2 and n+1 at step (3).

Preferably, the first strand is cleaved by cleavage of the phosphodiester bond between nucleotide positions n+2 and n+1 (the first phosphodiester bond of the first strand relative to the position of the universal nucleotide, in the direction distal to the ligated polynucleotide ligation molecule/proximal to the first strand).

The first strand may be cleaved by cleavage of one ester bond of the phosphodiester bond between nucleotide positions n+2 and n+1.

Preferably the first strand is cleaved by cleavage of the first ester bond relative to nucleotide position n+2.

Any suitable mechanism may be employed to effect cleavage of the first strand between nucleotide positions n+2 and n+1 when the universal nucleotide occupies position n+2.

Cleavage of the first strand between nucleotide positions n+2 and n+1 as described above may be performed by the action of an enzyme.

Cleavage of the first strand between nucleotide positions n+2 and n+1 as described above may be performed as a two-step cleavage process.

The first cleavage step of a two-step cleavage process may comprise removing the universal nucleotide from the first strand thus forming an abasic site at position n+2, and the second cleavage step may comprise cleaving the first strand at the abasic site, between positions n+2 and n+1.

One mechanism of cleaving the first strand at a cleavage site defined by a sequence comprising a universal nucleotide in the manner outlined above is described in analogous fashion in Example 2. The cleavage mechanism described in Example 2 is exemplary and other mechanisms could be employed, provided that the cleaved double-stranded scaffold polynucleotide described above is achieved.

In the first cleavage step of a two-step cleavage process the universal nucleotide is removed from the first strand whilst leaving the sugar-phosphate backbone intact. This can be achieved by the action of an enzyme which can specifically excise a single universal nucleotide from a double-stranded polynucleotide. In the exemplified cleavage methods the universal nucleotide is inosine and inosine is excised from the first strand by the action of an enzyme, thus forming an abasic site. In the exemplified cleavage method the enzyme is a 3-methyladenine DNA glycosylase enzyme, specifically human alkyladenine DNA glycosylase (hAAG). Other enzymes, molecules or chemicals could be used provided that an abasic site is formed. The nucleotide-excising enzyme may be an enzyme which catalyses the release of uracil from polynucleotides, such as Uracil-DNA Glycosylase (UDG).

In the second step of a two-step cleavage process the first strand is cleaved at the abasic site by making a single-strand break. In the exemplified methods the first strand is cleaved by the action of a chemical which is a base, such as NaOH. Alternatively, an organic chemical such as N,N′-dimethylethylenediamine may be used. Alternatively, enzymes having abasic site lyase activity, such as AP Endonuclease 1, Endonuclease III (Nth), or Endonuclease VIII, may be used. These enzymes cleave the DNA backbone at a phosphate group which is positioned 5′ relative to the abasic site. Cleavage thus exposes an OH group at the 3′ terminal end of the first strand which provides a terminal 3′ nucleotide which is ligatable in the first ligation step in the next cycle. Other enzymes, molecules or chemicals could be used provided that the first strand is cleaved at the abasic site as described.

Thus in embodiments wherein the universal nucleotide is at position n+2 of the first strand at step (2) and the first strand is cleaved between positions n+2 and n+1, a first cleavage step may be performed with a nucleotide-excising enzyme. An example of such an enzyme is a 3-methyladenine DNA glycosylase enzyme, such as human alkyladenine DNA glycosylase (hAAG). The second cleavage step may be performed with a chemical which is a base, such as NaOH. The second step may be performed with an organic chemical having abasic site cleavage activity such as N,N′-dimethylethylenediamine. The second step may be performed with an enzyme having abasic site lyase activity such as Endonuclease VIII or Endonuclease III.

Cleavage of the first strand between nucleotide positions n+2 and n+1 as described above may also be performed as a one-step cleavage process. Examples of enzymes which may be used in any such process include Endonuclease III, Endonuclease VIII. Other enzymes which may be used in any such process include enzymes which cleave 8-oxoguanosine, such as formamidopyrimidine DNA glycosylase (Fpg) and 8-oxoguanine DNA glycosylase (hOGG1), which cleaves the DNA backbone so as to leave a phosphate group at the 3′ terminal end of the cleaved first strand, which can then be removed by Endonuclease IV or T4 polynucleotide kinase to expose an OH group which is ligatable in the first ligation step in the next cycle.

### Step 4—Ligation of a Second Polynucleotide Ligation Molecule to the Scaffold Polynucleotide and Incorporation of One or More Further Nucleotides of the Predefined Sequence

In step (4) of the method a second double-stranded polynucleotide ligation molecule is ligated (204) to the scaffold polynucleotide in a sticky—(complementary)—ended ligation reaction by the action of an enzyme having ligase activity.

The second polynucleotide ligation molecule comprises a synthesis strand and a helper strand hybridised thereto. The second polynucleotide ligation molecule further comprises a complementary ligation end comprising in the synthesis strand a universal nucleotide and two further nucleotides of the predefined sequence.

The complementary ligation end of the second polynucleotide ligation molecule is structured such that the terminal and penultimate nucleotides of the synthesis strand are respectively the first and second nucleotides of the predefined sequence to be incorporated into the second strand of the scaffold polynucleotide in any given cycle of synthesis.

The complementary ligation end comprises a single-base overhang. The terminal nucleotide of the synthesis strand is unpaired and overhangs the terminal nucleotide of the helper strand. The penultimate nucleotide of the synthesis strand is paired with the terminal nucleotide of the helper strand. In FIG. 2 the terminal nucleotide of the synthesis strand is depicted as “C”, the penultimate nucleotide of the synthesis strand is depicted as “T”, and the terminal nucleotide of the helper strand is depicted as “A”. Each of these designations are for illustrative purposes only. They can be any nucleotide, nucleotide analogue or nucleotide derivative. The penultimate nucleotide of the synthesis strand and the terminal nucleotide of the helper strand may or may not be complementary. Preferably they are complementary.

At the terminal end of the synthesis strand at the complementary ligation end of the second polynucleotide ligation molecule, the universal nucleotide occupies position n+4, which is the third position removed from the terminal nucleotide of the synthesis strand in the direction distal to the complementary ligation end, the terminal nucleotide occupying position n+1 and the penultimate nucleotide occupying position n+2. With reference to the definition of position n in the first extension/ligation reaction, position n refers to the nucleotide position in the second strand of the scaffold polynucleotide which is occupied by a partner nucleotide for the first nucleotide attached to the terminal end of the first strand after the first extension/ligation reaction.

The universal nucleotide forms a nucleotide pair with the nucleotide which occupies a position immediately next to the penultimate nucleotide of the helper strand in the direction distal to the complementary ligation end. This is depicted in FIG. 2 as “X”. It can be any nucleotide, nucleotide analogue or nucleotide derivative. In FIG. 2 the nucleotides occupying position n+3 in both the synthesis strand and the helper strand are depicted as “X” for illustrative purposes only. Each one of these nucleotides can be any nucleotide, nucleotide analogue or nucleotide derivative, and they may or may not be complementary.

The terminal nucleotide of the synthesis strand at the complementary ligation end of the second polynucleotide ligation molecule is depicted in FIG. 2 at the 3′ end of the synthesis strand. This nucleotide is provided as a ligatable nucleotide and comprises a hydroxyl group or any other suitable 3′ ligatable group. The terminal nucleotide of the helper strand at the complementary ligation end of the second polynucleotide ligation molecule is depicted in FIG. 2 at the 5′ end of the helper strand. This nucleotide is provided as a non-ligatable nucleotide, e.g. lacking a phosphate group or provided with any suitable 5′ blocking group which can prevent ligation.

The complementary ligation end of the second polynucleotide ligation molecule is configured so that it will compatibly join with the overhanging end of the cleaved scaffold polynucleotide, generated in step (3), when subjected to suitable ligation conditions. Upon ligation of the synthesis strand of the second polynucleotide ligation molecule and the second strand of the cleaved scaffold polynucleotide, the terminal and penultimate nucleotides of the synthesis strand of the second polynucleotide ligation become incorporated into the second strand of the cleaved scaffold polynucleotide. Because the terminal nucleotide of the helper strand of the second polynucleotide ligation molecule is a non-ligatable nucleotide, the ligase enzyme will be prevented from ligating the helper strand of the second polynucleotide ligation molecule and the first strand of the cleaved scaffold polynucleotide, thus creating a single-strand break or “nick” between the helper strand of the second polynucleotide ligation molecule and the first strand of the cleaved scaffold polynucleotide.

Ligation of the second polynucleotide ligation molecule to the cleaved scaffold polynucleotide extends the length of the second strand of the double-stranded scaffold polynucleotide of step (3) and wherein the terminal and penultimate nucleotides of the synthesis strand of the second polynucleotide ligation molecule sequence are incorporated into the second strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

Upon ligation, the terminal nucleotide of the synthesis strand of the second polynucleotide ligation molecule pairs with the terminal nucleotide of the first strand of the cleaved scaffold polynucleotide generated in step (3), thus forming a nucleotide pair.

### Step 5—Second Cleavage Step

In step (5) of the method the ligated scaffold polynucleotide is cleaved (205) at a cleavage site. The cleavage site is defined by a sequence comprising the universal nucleotide in the synthesis strand of the ligated second polynucleotide ligation molecule. Cleavage results in a double-strand break in the scaffold polynucleotide. Cleavage of the scaffold polynucleotide (step 5) results in loss of the helper strand of the ligated second polynucleotide ligation molecule and loss of the synthesis strand comprising the universal nucleotide. Cleavage of the scaffold polynucleotide thereby releases the second polynucleotide ligation molecule from the scaffold polynucleotide but leads to the retention of two nucleotides attached to the second strand of the cleaved scaffold polynucleotide. Cleavage of the scaffold polynucleotide leaves in place a cleaved double-stranded scaffold polynucleotide comprising a singe-base overhang at the cleaved end. The second nucleotide of the predefined sequence incorporated in step (4) occupies a position (n+2) as the terminal nucleotide of the second strand of the cleaved double-stranded scaffold polynucleotide, and the first nucleotide of the predefined sequence incorporated in step (4) occupies a position (n+1) as the penultimate nucleotide of the cleaved second strand. The first nucleotide of the predefined sequence incorporated in step (4) is paired with the nucleotide which was the terminal overhanging nucleotide of the first strand in the double-stranded scaffold polynucleotide in step (3) (203) following the first cleavage step. In FIG. 2 these nucleotides are depicted (205) as “C” and “G” for illustrative purposes only. Each one of these nucleotides can be any nucleotide, nucleotide analogue or nucleotide derivative and the pair may or may not be complementary. Preferably they are complementary.

Prior to the second cleavage step the first strand of the ligated scaffold polynucleotide is already provided with a single-strand break or “nick” in this exemplary method, thus only cleavage of the second strand is necessary to provide a double-strand break in the scaffold polynucleotide. Furthermore, as noted previously in this exemplary method version cleavage generates a cleaved double-stranded scaffold polynucleotide with a single-base overhang, with the terminal nucleotide of the second strand overhanging the terminal nucleotide of the first strand, and the universal nucleotide occupies position n+4 in the second strand prior to the cleavage step. To obtain such a cleaved double-stranded scaffold polynucleotide with a singe-base overhang when the universal nucleotide occupies position n+4 in the second strand, the second strand is cleaved at a specific position relative to the universal nucleotide. When the second strand of the scaffold polynucleotide is cleaved between nucleotide positions n+3 and n+2 the polynucleotide ligation molecule is released from the scaffold polynucleotide (see the structure depicted as exiting the synthesis cycle immediately after cleavage step 5 (205) in FIG. 2) except that the first and second nucleotides of that cycle derived from the second polynucleotide ligation molecule in step (4) are retained in the scaffold polynucleotide attached to the second strand of the cleaved scaffold polynucleotide.

A phosphate group, or any other suitable 5′ ligatable group, should continue to be attached to the terminal nucleotide of the second strand of the cleaved scaffold polynucleotide at the cleavage site. This ensures that the second strand of the cleaved scaffold polynucleotide can be ligated to the synthesis strand of the second polynucleotide ligation molecule in the second extension/ligation step (4) of the next cycle of synthesis. Cleavage is performed so that the terminal nucleotide of the first strand of the cleaved scaffold polynucleotide retains a ligatable group, typically a hydroxyl group or any other suitable 3′ ligatable group, at the 3′ end of the first strand.

Thus in method version 2 the universal nucleotide occupies position n+4 in the synthesis/second strand at step (4) and the second strand is cleaved between nucleotide positions n+3 and n+2 at step (5).

Preferably, the second strand is cleaved by cleavage of the phosphodiester bond between nucleotide positions n+3 and n+2 (the first phosphodiester bond of the second strand relative to the position of the universal nucleotide, in the direction distal to the ligated polynucleotide ligation molecule/proximal to the second strand).

The second strand may be cleaved by cleavage of one ester bond of the phosphodiester bond between nucleotide positions n+3 and n+2.

Preferably the second strand is cleaved by cleavage of the first ester bond relative to nucleotide position n+4.

Any suitable mechanism may be employed to effect cleavage of the second strand between nucleotide positions n+3 and n+2 when the universal nucleotide occupies position n+4.

Cleavage of the second strand between nucleotide positions n+3 and n+2 as described above may be performed by the action of an enzyme.

Cleavage of the second strand between nucleotide positions n+3 and n+2 when the universal nucleotide occupies position n+4 in the second strand, as described above, may be performed by the action of an enzyme such as Endonuclease V.

One mechanism of cleaving the second strand between nucleotide positions n+3 and n+2 at a cleavage site defined by a sequence comprising a universal nucleotide which is occupying position n+4 in the second strand is described in analogous fashion in Example 3. The mechanism described is exemplary and other mechanisms could be employed, provided that the cleavage arrangement described above is achieved.

In this exemplary mechanism an endonuclease enzyme is employed. In the exemplified method the enzyme is Endonuclease V. Other enzymes, molecules or chemicals could be used provided that the second strand is cleaved between nucleotide positions n+3 and n+2 when the universal nucleotide occupies position n+4 in the second strand.

In synthesis method version 2 it will be noted that in any given cycle of synthesis, after the second cleavage step (step 5) the nucleotide position which is occupied by the terminal nucleotide of the second strand at the cleaved end is defined as nucleotide position n+2. This nucleotide position is defined as nucleotide position n in the next cycle of synthesis. Similarly, the nucleotide position which is occupied by the terminal nucleotide of the first strand at the cleaved end is defined as nucleotide position n+1. This nucleotide position is defined as nucleotide position n−1 in the next cycle of synthesis.

### Further Cycles

Following completion of the first cycle of synthesis, second and further cycles of synthesis may be performed using the same method steps.

The cleavage product of step (5) of the previous cycle is provided (in step 6) as the double-stranded scaffold polynucleotide for the next cycle of synthesis.

In step (7) of the next and each further cycle of synthesis a further first double-stranded polynucleotide ligation molecule is ligated to the cleavage product of step (5) of the previous cycle. The polynucleotide ligation molecule may be structured in the same way as described above for step (2) of the previous cycle, except that the further first polynucleotide ligation molecule comprises further first and second nucleotides of the further cycle of synthesis to be incorporated into the first strand. In step (7) the further first polynucleotide ligation molecule may be ligated to the cleavage product of step (5) of the previous cycle in the same way as described above for step (2).

In step (8) of the next and each further cycle of synthesis the ligated scaffold polynucleotide is subjected to a further first cleavage step at the cleavage site. Cleavage results in a double-strand break in the scaffold polynucleotide. Cleavage of the scaffold polynucleotide (step 8) results in loss of the helper strand and loss of the support strand comprising the universal nucleotide in the further first polynucleotide ligation molecule. Cleavage of the scaffold polynucleotide thereby releases the further first polynucleotide ligation molecule from the scaffold polynucleotide but leads to the retention of the further first and second nucleotides of that further cycle derived from the further first polynucleotide ligation molecule attached to the synthesis strand of the cleaved scaffold polynucleotide. Cleavage of the scaffold polynucleotide leaves in place a cleaved double-stranded scaffold polynucleotide comprising the further first and second nucleotides of the further cycle at the terminal end of the synthesis strand of the scaffold polynucleotide. Cleavage results in a single-base overhang with the terminal nucleotide of the first strand overhanging the terminal nucleotide of the second strand. Cleavage at step (8) may be performed in the same way as described above for step (4).

In step (9) of the next and each further cycle of synthesis a further second double-stranded polynucleotide ligation molecule is ligated to the cleavage product of step (8). The further second polynucleotide ligation molecule may be structured in the same way as described above for step (8) of the previous cycle, except that the further second polynucleotide ligation molecule comprises further first and second nucleotides of the further cycle of synthesis to be incorporated into the second strand. In step (9) the further second polynucleotide ligation molecule may be ligated to the cleavage product of step (8) in the same way as described above for step (4).

In step (10) of the next and each further cycle of synthesis the ligated scaffold polynucleotide is subjected to a further second cleavage step at the cleavage site. Cleavage results in a double-strand break in the scaffold polynucleotide. Cleavage of the scaffold polynucleotide (step 10) results in loss of the helper strand and loss of the support strand comprising the universal nucleotide in the further second polynucleotide ligation molecule. Cleavage of the scaffold polynucleotide thereby releases the further second polynucleotide ligation molecule from the scaffold polynucleotide but leads to the retention of the further first and second nucleotides of that further cycle derived from the further second polynucleotide ligation molecule attached to the synthesis strand of the cleaved scaffold polynucleotide. Cleavage of the scaffold polynucleotide leaves in place a cleaved double-stranded scaffold polynucleotide comprising the further first and second nucleotides of the further cycle derived from the further second polynucleotide ligation molecule at the terminal end of the second strand of the scaffold polynucleotide. The further second nucleotide is the terminal nucleotide of the second strand and overhangs the terminal nucleotide of the first strand in a single-base overhang. Cleavage at step (10) may be performed in the same way as described above for step (5).

### Synthesis Method Version 3

### Step 1—Provision of a Scaffold Polynucleotide

In exemplary version 3 of the synthesis methods of the invention a double-stranded scaffold polynucleotide is provided in step (1) (301). The double-stranded scaffold polynucleotide is provided comprising a first strand and a second strand hybridised thereto. The terminal nucleotide of the first strand at the end to be extended is positioned at the 3′ end of the first strand and comprises a hydroxyl group or any other suitable 3′ ligatable group and therefore this terminal nucleotide is a ligatable nucleotide. In FIG. 3 this nucleotide is depicted as “X” and can be any nucleotide, nucleotide analogue or nucleotide derivative. The terminal nucleotide of the first strand at the end to be extended is depicted as paired with the terminal nucleotide of the 5′ end of the second strand. This terminal nucleotide is depicted as “X” and can be any nucleotide, nucleotide analogue or nucleotide derivative, and may or may not be complementary to its partner nucleotide in the pair. Preferably it is complementary. Since the terminal nucleotide of the 5′ end of the second strand is paired with the terminal 3′ nucleotide of the first strand, the end of the scaffold polynucleotide to be extended is blunt-ended with no overhanging nucleotides. The terminal nucleotide of the 5′ end of the second strand comprises a phosphate group or any other suitable 5′ ligatable group, and therefore this terminal nucleotide is also a ligatable nucleotide.

The terminal ends of the scaffold polynucleotide which are not shown to be extended, i.e. those labelled 3′ and 5′ in FIG. 3, are preferably attached to a substrate, such as by any of the means described herein.

### Step 2—Ligation of a First Polynucleotide Ligation Molecule to the Scaffold Polynucleotide and Incorporation of One or More Nucleotides of the Predefined Sequence

In step (2) of the method a double-stranded polynucleotide ligation molecule is ligated (302) to the scaffold polynucleotide in a blunt-ended ligation reaction by the action of an enzyme having ligase activity.

The polynucleotide ligation molecule comprises a synthesis strand and a helper strand hybridised thereto. The polynucleotide ligation molecule further comprises a complementary ligation end comprising in the synthesis strand a universal nucleotide and a nucleotide of the predefined sequence.

The complementary ligation end of the first polynucleotide ligation molecule is structured such that the terminal nucleotide of the synthesis strand is the first nucleotide of the predefined sequence to be incorporated into the scaffold polynucleotide in any given cycle of synthesis. The terminal nucleotide of the synthesis strand is paired with the terminal nucleotide of the helper strand. In FIG. 3 the terminal nucleotide of the synthesis strand is depicted as “T” and the terminal nucleotide of the helper strand is depicted as “A”. Each of these designations are for illustrative purposes only. They can be any nucleotide, nucleotide analogue or nucleotide derivative, and they may or may not be complementary. Preferably they are complementary.

The universal nucleotide is the penultimate nucleotide at the terminal end of the synthesis strand at the complementary ligation end of the first polynucleotide ligation molecule. The universal nucleotide forms a nucleotide pair with the penultimate nucleotide at the terminal end of the helper strand at the complementary ligation end of the first polynucleotide ligation molecule. In FIG. 3 the penultimate nucleotide at the terminal end of the helper strand at the complementary ligation end is depicted as “X” for illustrative purposes only. It can be any nucleotide, nucleotide analogue or nucleotide derivative.

The terminal nucleotide of the synthesis strand at the complementary ligation end of the first polynucleotide ligation molecule is depicted in FIG. 3 at the 5′ end of the synthesis strand. This nucleotide is a ligatable nucleotide and is provided with a phosphate group or any other suitable 5′ ligatable group. The terminal nucleotide of the helper strand at the complementary ligation end of the first polynucleotide ligation molecule is depicted in FIG. 3 at the 3′ end of the helper strand. This nucleotide is provided as a non-ligatable nucleotide and comprises a non-ligatable 2′,3′-dideoxynucleotide or a 2′-deoxynucleotide, or any other suitable non-ligatable nucleotide.

The terminal nucleotide of the synthesis strand, i.e. the first nucleotide of the predefined sequence to be incorporated into the first strand in a given cycle of synthesis, occupies nucleotide position n in the synthesis strand. By position n in the synthesis strand of the first polynucleotide ligation molecule it is meant the position which will be occupied by the first nucleotide which is to be attached to the terminal end of the first strand of the scaffold polynucleotide following ligation of the polynucleotide ligation molecule to the scaffold polynucleotide. Position n also refers to the nucleotide position in the first strand of the ligated scaffold polynucleotide which is occupied by the said first nucleotide following its attachment to the terminal end of the first strand after ligation. Position n also refers to the nucleotide position in the second strand of the scaffold polynucleotide which will be occupied by a partner nucleotide for the said first nucleotide following attachment of the partner nucleotide to the terminal end of the second strand after the second extension/ligation reaction. Position n also refers to the nucleotide position which is occupied by the said partner nucleotide following its attachment to the terminal end of the second strand after the second extension/ligation reaction. The universal nucleotide in the synthesis strand of the first polynucleotide ligation molecule occupies position n+1.

The complementary ligation end of the first polynucleotide ligation molecule is configured so that it will compatibly join with the blunt end of the scaffold polynucleotide when subjected to suitable ligation conditions. Upon ligation of the synthesis strand of the polynucleotide ligation molecule and the first strand of the scaffold polynucleotide, the terminal nucleotide of the synthesis strand becomes incorporated into the first strand of the scaffold polynucleotide. Because the terminal nucleotide of the helper strand of the polynucleotide ligation molecule is a non-ligatable nucleotide, the ligase enzyme will be prevented from ligating the helper strand of the first polynucleotide ligation molecule and the second strand of the scaffold polynucleotide, thus creating a single-strand break or “nick” between the helper strand of the first polynucleotide ligation molecule and the second strand of the scaffold polynucleotide.

Ligation of the polynucleotide ligation molecule to the scaffold polynucleotide extends the length of the first strand of the double-stranded scaffold polynucleotide of step (1) and wherein the terminal nucleotide of the synthesis strand of the first polynucleotide ligation molecule is incorporated into the first strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

### Step 3—First Cleavage Step

In step (3) of the method the ligated scaffold polynucleotide is cleaved (303) at a cleavage site. The cleavage site is defined by a sequence comprising the universal nucleotide in the synthesis strand of the ligated first polynucleotide ligation molecule. Cleavage results in a double-strand break in the scaffold polynucleotide. Cleavage of the scaffold polynucleotide (step 3) results in loss of the helper strand of the ligated first polynucleotide ligation molecule and loss of the synthesis strand comprising the universal nucleotide. Cleavage of the scaffold polynucleotide thereby releases the polynucleotide ligation molecule from the scaffold polynucleotide but leads to the retention of the first nucleotide of that cycle attached to the first strand of the cleaved scaffold polynucleotide. Cleavage of the scaffold polynucleotide leaves in place a cleaved double-stranded scaffold polynucleotide comprising a singe-base overhang at the cleaved end. The first nucleotide of the predefined sequence occupies a position (n) as the terminal nucleotide of the first strand of the cleaved double-stranded scaffold polynucleotide and overhangs the terminal nucleotide of the second strand at the cleaved end.

The second strand of the ligated scaffold polynucleotide is already provided with a single-strand break or “nick” in this exemplary method, thus only cleavage of the first strand is necessary to provide a double-strand break in the scaffold polynucleotide. Furthermore, as noted previously, in this exemplary method version cleavage generates a cleaved double-stranded scaffold polynucleotide with a single-base overhang, with the terminal nucleotide of the first strand overhanging the terminal nucleotide of the second strand, and the universal nucleotide occupies position n+1 in the first strand prior to the cleavage step. To obtain such a cleaved double-stranded scaffold polynucleotide with a single-base overhang when the universal nucleotide occupies position n+1 in the first strand, the first strand is cleaved at a specific position relative to the universal nucleotide. When the first strand of the scaffold polynucleotide is cleaved between nucleotide positions n+1 and n the polynucleotide ligation molecule is released from the scaffold polynucleotide (see the structure depicted as exiting the synthesis cycle immediately after cleavage step 3 (303) in FIG. 3) except that the first nucleotide of that cycle derived from the first polynucleotide ligation molecule in step (2) is retained in the scaffold polynucleotide attached to the first strand of the cleaved scaffold polynucleotide.

A phosphate group, or any other suitable 5′ ligatable group, should continue to be attached to the terminal nucleotide of the second strand of the cleaved scaffold polynucleotide at the cleavage site. This ensures that the second strand of the cleaved scaffold polynucleotide can be ligated to the synthesis strand of the second polynucleotide ligation molecule in the second extension/ligation step (4). Cleavage is performed so that the terminal nucleotide of the first strand of the cleaved scaffold polynucleotide retains a ligatable group, typically a hydroxyl group or any other suitable 3′ ligatable group, at the 3′ end of the first strand.

Thus in method version 3 the universal nucleotide occupies position n+1 in the synthesis/first strand at step (2) and the first strand is cleaved between nucleotide positions n+1 and n at step (3).

Preferably, the first strand is cleaved by cleavage of the phosphodiester bond between nucleotide positions n+1 and n (the first phosphodiester bond of the first strand relative to the position of the universal nucleotide, in the direction distal to the ligated polynucleotide ligation molecule/proximal to the first strand).

The first strand may be cleaved by cleavage of one ester bond of the phosphodiester bond between nucleotide positions n+1 and n.

Preferably the first strand is cleaved by cleavage of the first ester bond relative to nucleotide position n+1.

Any suitable mechanism may be employed to effect cleavage of the first strand between nucleotide positions n+1 and n when the universal nucleotide occupies position n+1.

Cleavage of the first strand between nucleotide positions n+1 and n as described above may be performed by the action of an enzyme.

Cleavage of the first strand between nucleotide positions n+1 and n as described above may be performed as a two-step cleavage process.

The first cleavage step of a two-step cleavage process may comprise removing the universal nucleotide from the first strand thus forming an abasic site at position n+1, and the second cleavage step may comprise cleaving the first strand at the abasic site, between positions n+1 and n.

One mechanism of cleaving the first strand at a cleavage site defined by a sequence comprising a universal nucleotide in the manner outlined above is described in analogous fashion in Example 2. The cleavage mechanism described in Example 2 is exemplary and other mechanisms could be employed, provided that the cleaved double-stranded scaffold polynucleotide described above is achieved.

In the first cleavage step of a two-step cleavage process the universal nucleotide is removed from the first strand whilst leaving the sugar-phosphate backbone intact. This can be achieved by the action of an enzyme which can specifically excise a single universal nucleotide from a double-stranded polynucleotide. In the exemplified cleavage methods the universal nucleotide is inosine and inosine is excised from the first strand by the action of an enzyme, thus forming an abasic site. In the exemplified cleavage method the enzyme is a 3-methyladenine DNA glycosylase enzyme, specifically human alkyladenine DNA glycosylase (hAAG). Other enzymes, molecules or chemicals could be used provided that an abasic site is formed. The nucleotide-excising enzyme may be an enzyme which catalyses the release of uracil from polynucleotides, such as Uracil-DNA Glycosylase (UDG).

In the second step of a two-step cleavage process the first strand is cleaved at the abasic site by making a single-strand break. In the exemplified methods the first strand is cleaved by the action of a chemical which is a base, such as NaOH. Alternatively, an organic chemical such as N,N′-dimethylethylenediamine may be used. Alternatively, enzymes having abasic site lyase activity, such as AP Endonuclease 1, Endonuclease III (Nth), or Endonuclease VIII, may be used. These enzymes cleave the DNA backbone at a phosphate group which is positioned 5′ relative to the abasic site. Cleavage thus exposes an OH group at the 3′ terminal end of the first strand which provides a terminal 3′ nucleotide which is ligatable in the first ligation step in the next cycle. Other enzymes, molecules or chemicals could be used provided that the first strand is cleaved at the abasic site as described.

Thus in embodiments wherein the universal nucleotide is at position n+1 of the first strand at step (2) and the first strand is cleaved between positions n+1 and n, a first cleavage step may be performed with a nucleotide-excising enzyme. An example of such an enzyme is a 3-methyladenine DNA glycosylase enzyme, such as human alkyladenine DNA glycosylase (hAAG). The second cleavage step may be performed with a chemical which is a base, such as NaOH. The second step may be performed with an organic chemical having abasic site cleavage activity such as N,N′-dimethylethylenediamine. The second step may be performed with an enzyme having abasic site lyase activity such as Endonuclease VIII or Endonuclease III.

Cleavage of the first strand between nucleotide positions n+1 and n as described above may also be performed as a one-step cleavage process. Examples of enzymes which may be used in any such process include Endonuclease III, Endonuclease VIII. Other enzymes which may be used in any such process include enzymes which cleave 8-oxoguanosine, such as formamidopyrimidine DNA glycosylase (Fpg) and 8-oxoguanine DNA glycosylase (hOGG1), which cleaves the DNA backbone so as to leave a phosphate group at the 3′ terminal end of the cleaved first strand, which can then be removed by Endonuclease IV or T4 polynucleotide kinase to expose an OH group which is ligatable in the first ligation step in the next cycle.

### Step 4—Ligation of a Second Polynucleotide Ligation Molecule to the Scaffold Polynucleotide and Incorporation of One or More Further Nucleotides of the Predefined Sequence

In step (4) of the method a second double-stranded polynucleotide ligation molecule is ligated (304) to the cleaved scaffold polynucleotide in a sticky-(complementary)-ended ligation reaction by the action of an enzyme having ligase activity.

The second polynucleotide ligation molecule comprises a synthesis strand and a helper strand hybridised thereto. The second polynucleotide ligation molecule further comprises a complementary ligation end comprising in the synthesis strand a universal nucleotide and a further nucleotide of the predefined sequence.

The complementary ligation end of the second polynucleotide ligation molecule is structured such that the terminal nucleotide of the synthesis strand is the first nucleotide of the predefined sequence to be incorporated into the second strand of the cleaved scaffold polynucleotide in any given cycle of synthesis.

The complementary ligation end comprises a single-base overhang. The terminal nucleotide of the synthesis strand is unpaired and overhangs the terminal nucleotide of the helper strand. In FIG. 3 the terminal nucleotide of the synthesis strand is depicted as “A” for illustrative purposes only. It can be any nucleotide, nucleotide analogue or nucleotide derivative.

The universal nucleotide is the penultimate nucleotide at the terminal end of the synthesis strand at the complementary ligation end of the second polynucleotide ligation molecule. The universal nucleotide forms a nucleotide pair with the terminal nucleotide of the helper strand at the complementary ligation end of the second polynucleotide ligation molecule. In FIG. 3 the terminal nucleotide of the helper strand at the complementary ligation end is depicted as “X” for illustrative purposes only. It can be any nucleotide, nucleotide analogue or nucleotide derivative.

The terminal nucleotide of the synthesis strand at the complementary ligation end of the second polynucleotide ligation molecule is depicted in FIG. 3 at the 3′ end of the synthesis strand. This nucleotide is a ligatable nucleotide and is provided with a hydroxyl group or any other suitable 3′ ligatable group. The terminal nucleotide of the helper strand at the complementary ligation end of the second polynucleotide ligation molecule is depicted in FIG. 3 at the 5′ end of the helper strand. This nucleotide is provided as a non-ligatable nucleotide, e.g. lacking a phosphate group or provided with any suitable 5′ blocking group which can prevent ligation.

The terminal nucleotide of the synthesis strand, i.e. the first nucleotide of the predefined sequence of that cycle to be incorporated into the second strand of the cleaved scaffold polynucleotide, occupies nucleotide position n in the synthesis strand. With reference to the definition of position n in the first extension/ligation reaction, position n refers to the nucleotide position in the synthesis strand of the second polynucleotide ligation molecule which is occupied by a nucleotide which will be a partner nucleotide for the first nucleotide of step (2) in the first strand following attachment of the partner nucleotide to the terminal end of the second strand after the second extension/ligation reaction. Position n also refers to the nucleotide position which is occupied by the said partner nucleotide following its attachment to the terminal end of the second strand after the second extension/ligation reaction. Position n also refers to the nucleotide position in the first strand of the scaffold polynucleotide which is occupied by the first nucleotide following its attachment to the terminal end of the first strand after ligation in step (2).

The universal nucleotide in the synthesis strand of the second polynucleotide ligation molecule occupies position n+1.

The complementary ligation end of the second polynucleotide ligation molecule is configured so that it will compatibly join with the overhanging end of the cleaved scaffold polynucleotide, generated in step (3), when subjected to suitable ligation conditions. Upon ligation of the synthesis strand of the second polynucleotide ligation molecule and the second strand of the scaffold polynucleotide, the terminal nucleotide of the synthesis strand of the second polynucleotide ligation becomes incorporated into the second strand of the cleaved scaffold polynucleotide. Because the terminal nucleotide of the helper strand of the second polynucleotide ligation molecule is a non-ligatable nucleotide, the ligase enzyme will be prevented from ligating the helper strand of the second polynucleotide ligation molecule and the first strand of the cleaved scaffold polynucleotide, thus creating a single-strand break or “nick” between the helper strand of the second polynucleotide ligation molecule and the first strand of the cleaved scaffold polynucleotide.

Ligation of the second polynucleotide ligation molecule to the cleaved scaffold polynucleotide extends the length of the second strand of the double-stranded scaffold polynucleotide of step (3) and wherein the terminal nucleotide of the synthesis strand of the second polynucleotide ligation molecule sequence is incorporated into the second strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

Upon ligation, the terminal nucleotide of the synthesis strand of the second polynucleotide ligation molecule pairs with the terminal nucleotide of the first strand of the cleaved scaffold polynucleotide generated in step (3), thus forming a nucleotide pair.

### Step 5—Second Cleavage Step

In step (5) of the method the ligated scaffold polynucleotide is cleaved (305) at a cleavage site. The cleavage site is defined by a sequence comprising the universal nucleotide in the synthesis strand of the ligated second polynucleotide ligation molecule. Cleavage results in a double-strand break in the scaffold polynucleotide. Cleavage of the scaffold polynucleotide (step 5) results in loss of the helper strand of the ligated second polynucleotide ligation molecule and loss of the synthesis strand comprising the universal nucleotide. Cleavage of the scaffold polynucleotide thereby releases the second polynucleotide ligation molecule from the scaffold polynucleotide but leads to the retention of the first nucleotide of that cycle attached to the second strand of the cleaved scaffold polynucleotide. Cleavage of the scaffold polynucleotide leaves in place a cleaved double-stranded scaffold polynucleotide comprising a blunt end. The second nucleotide of the predefined sequence incorporated in step (4) occupies a position (n) as the terminal nucleotide of the second strand of the cleaved double-stranded scaffold polynucleotide and is paired with the first/terminal nucleotide of the first strand of the cleaved double-stranded scaffold polynucleotide which was incorporated in step (2) and which occupies position n in the first strand. In FIG. 3 these nucleotides are depicted (305) as “A” and “T” for illustrative purposes only. Each of these nucleotides can be any nucleotide, nucleotide analogue or nucleotide derivative and the pair may or may not be complementary. Preferably they are complementary.

Prior to the second cleavage step the first strand of the ligated scaffold polynucleotide is already provided with a single-strand break or “nick” in this exemplary method, thus only cleavage of the second strand is necessary to provide a double-strand break in the scaffold polynucleotide. Furthermore, as noted previously in this exemplary method version cleavage generates a cleaved double-stranded scaffold polynucleotide with a blunt end, and the universal nucleotide occupies position n+1 in the second strand prior to the second cleavage step. To obtain such a cleaved double-stranded scaffold polynucleotide with a blunt end when the universal nucleotide occupies position n+1 in the second strand, the second strand is cleaved at a specific position relative to the universal nucleotide. When the second strand of the scaffold polynucleotide is cleaved between nucleotide positions n+1 and n the polynucleotide ligation molecule is released from the scaffold polynucleotide (see the structure depicted as exiting the synthesis cycle immediately after cleavage step 5 (305) in FIG. 3) except that the first nucleotide of that cycle derived from the second polynucleotide ligation molecule in step (4) is retained in the scaffold polynucleotide attached to the second strand of the cleaved scaffold polynucleotide.

A phosphate group, or any other suitable 5′ ligatable group, should continue to be attached to the terminal nucleotide of the second strand of the cleaved scaffold polynucleotide at the cleavage site. This ensures that the second strand of the cleaved scaffold polynucleotide can be ligated to the synthesis strand of the second polynucleotide ligation molecule in the second extension/ligation step (4) of the next cycle of synthesis. Cleavage is performed so that the terminal nucleotide of the first strand of the cleaved scaffold polynucleotide retains a ligatable group, typically a hydroxyl group or any other suitable 3′ ligatable group, at the 3′ end of the first strand.

Thus in method version 3 the universal nucleotide occupies position n+1 in the synthesis/second strand at step (4) and the second strand is cleaved between nucleotide positions n+1 and n at step (5).

Preferably, the second strand is cleaved by cleavage of the phosphodiester bond between nucleotide positions n+1 and n (the first phosphodiester bond of the second strand relative to the position of the universal nucleotide, in the direction distal to the ligated polynucleotide ligation molecule/proximal to the second strand).

The second strand may be cleaved by cleavage of one ester bond of the phosphodiester bond between nucleotide positions n+1 and n.

Preferably the second strand is cleaved by cleavage of the first ester bond relative to nucleotide position n+1.

Any suitable mechanism may be employed to effect cleavage of the second strand between nucleotide positions n+1 and n when the universal nucleotide occupies position n+1.

Cleavage of the second strand between nucleotide positions n+1 and n as described above may be performed by the action of an enzyme.

Cleavage of the second strand between nucleotide positions n+1 and n as described above may be performed as a two-step cleavage process.

The first cleavage step of a two-step cleavage process may comprise removing the universal nucleotide from the second strand thus forming an abasic site at position n+1, and the second cleavage step may comprise cleaving the second strand at the abasic site, between positions n+1 and n.

One mechanism of cleaving the second strand at a cleavage site defined by a sequence comprising a universal nucleotide in the manner outlined above is described in analogous fashion in Example 2. The cleavage mechanism described in Example 2 is exemplary and other mechanisms could be employed, provided that the cleaved double-stranded scaffold polynucleotide described above is achieved.

In the first cleavage step of a two-step cleavage process the universal nucleotide is removed from the second strand whilst leaving the sugar-phosphate backbone intact. This can be achieved by the action of an enzyme which can specifically excise a single universal nucleotide from a double-stranded polynucleotide. In the exemplified cleavage methods the universal nucleotide is inosine and inosine is excised from the strand by the action of an enzyme, thus forming an abasic site. In the exemplified cleavage method the enzyme is a 3-methyladenine DNA glycosylase enzyme, specifically human alkyladenine DNA glycosylase (hAAG). Other enzymes, molecules or chemicals could be used provided that an abasic site is formed. The nucleotide-excising enzyme may be an enzyme which catalyses the release of uracil from polynucleotides, such as Uracil-DNA Glycosylase (UDG).

In the second step of a two-step cleavage process the second strand is cleaved at the abasic site by making a single-strand break. In the exemplified methods the strand is cleaved by the action of a chemical which is a base, such as NaOH. Alternatively, an organic chemical such as N,N′-dimethylethylenediamine may be used. Alternatively, enzymes having abasic site lyase activity, such as AP Endonuclease 1, Endonuclease III (Nth), or Endonuclease VIII, may be used. Other enzymes, molecules or chemicals could be used provided that the second strand is cleaved at the abasic site as described.

Thus in embodiments wherein the universal nucleotide is at position n+1 of the second strand at step (4) and the second strand is cleaved between positions n+1 and n, a first cleavage step may be performed with a nucleotide-excising enzyme. An example of such an enzyme is a 3-methyladenine DNA glycosylase enzyme, such as human alkyladenine DNA glycosylase (hAAG). The second cleavage step may be performed with a chemical which is a base, such as NaOH. The second step may be performed with an organic chemical having abasic site cleavage activity such as N,N′-dimethylethylenediamine. The second step may be performed with an enzyme having abasic site lyase activity such as Endonuclease VIII or Endonuclease III.

Cleavage of the second strand between nucleotide positions n+1 and n as described above may also be performed as a one-step cleavage process. Examples of enzymes which may be used in any such process include Endonuclease III, Endonuclease VIII. Other enzymes which may be used in any such process include enzymes which cleave 8-oxoguanosine, such as formamidopyrimidine DNA glycosylase (Fpg) and 8-oxoguanine DNA glycosylase (hOGG1).

In synthesis method version 3 it will be noted that in any given cycle of synthesis, after the second cleavage step (step 5) the nucleotide positions which are occupied by the terminal nucleotide of the first and second strands at the cleaved end are both defined as nucleotide position n. These nucleotide positions are defined as nucleotide position n−1 in the next cycle of synthesis.

### Further Cycles

Following completion of the first cycle of synthesis, second and further cycles of synthesis may be performed using the same method steps.

The cleavage product of step (5) of the previous cycle is provided (in step 6) as the double-stranded scaffold polynucleotide for the next cycle of synthesis.

In step (7) of the next and each further cycle of synthesis a further first double-stranded polynucleotide ligation molecule is ligated to the cleavage product of step (5) of the previous cycle. The polynucleotide ligation molecule may be structured in the same way as described above for step (2) of the previous cycle, except that the further first polynucleotide ligation molecule comprises further first nd nucleotide of the further cycle of synthesis to be incorporated into the first strand. In step (7) the further first polynucleotide ligation molecule may be ligated to the cleavage product of step (5) of the previous cycle in the same way as described above for step (2).

In step (8) of the next and each further cycle of synthesis the ligated scaffold polynucleotide is subjected to a further first cleavage step at the cleavage site. Cleavage results in a double-strand break in the scaffold polynucleotide. Cleavage of the scaffold polynucleotide (step 8) results in loss of the helper strand and loss of the support strand comprising the universal nucleotide in the further first polynucleotide ligation molecule. Cleavage of the scaffold polynucleotide thereby releases the further first polynucleotide ligation molecule from the scaffold polynucleotide but leads to the retention of the further first nucleotide of that further cycle derived from the further first polynucleotide ligation molecule attached to the synthesis strand of the cleaved scaffold polynucleotide. Cleavage of the scaffold polynucleotide leaves in place a cleaved double-stranded scaffold polynucleotide comprising the further first nucleotide of the further cycle at the terminal end of the synthesis strand of the scaffold polynucleotide. Cleavage results in a single-base overhang with the terminal nucleotide of the first strand overhanging the terminal nucleotide of the second strand. Cleavage at step (8) may be performed in the same way as described above for step (4).

In step (9) of the next and each further cycle of synthesis a further second double-stranded polynucleotide ligation molecule is ligated to the cleavage product of step (8). The further second polynucleotide ligation molecule may be structured in the same way as described above for step (8) of the previous cycle, except that the further second polynucleotide ligation molecule comprises further first nucleotide of the further cycle of synthesis to be incorporated into the second strand. In step (9) the further second polynucleotide ligation molecule may be ligated to the cleavage product of step (8) in the same way as described above for step (4).

In step (10) of the next and each further cycle of synthesis the ligated scaffold polynucleotide is subjected to a further second cleavage step at the cleavage site. Cleavage results in a double-strand break in the scaffold polynucleotide. Cleavage of the scaffold polynucleotide (step 10) results in loss of the helper strand and loss of the support strand comprising the universal nucleotide in the further second polynucleotide ligation molecule. Cleavage of the scaffold polynucleotide thereby releases the further second polynucleotide ligation molecule from the scaffold polynucleotide but leads to the retention of the further first nucleotide of that further cycle derived from the further second polynucleotide ligation molecule attached to the synthesis strand of the cleaved scaffold polynucleotide. Cleavage of the scaffold polynucleotide leaves in place a cleaved blunt-ended double-stranded scaffold polynucleotide comprising the further first nucleotide of the further cycle derived from the further second polynucleotide ligation molecule at the terminal end of the second strand of the scaffold polynucleotide. Cleavage at step (10) may be performed in the same way as described above for step (5).

### Synthesis Method Version 4

### Step 1—Provision of a Scaffold Polynucleotide

In exemplary version 4 of the synthesis methods of the invention a double-stranded scaffold polynucleotide is provided in step (1) (401). The double-stranded scaffold polynucleotide is provided comprising a first strand and a second strand hybridised thereto. The terminal nucleotide of the first strand at the end to be extended is positioned at the 3′ end of the first strand and comprises a hydroxyl group or any other suitable 3′ ligatable group and therefore this terminal nucleotide is a ligatable nucleotide. In FIG. 4 this nucleotide is depicted as “X” and can be any nucleotide, nucleotide analogue or nucleotide derivative. The terminal nucleotide of the first strand at the end to be extended is depicted as paired with the terminal nucleotide of the 5′ end of the second strand. This terminal nucleotide is depicted as “X” and can be any nucleotide, nucleotide analogue or nucleotide derivative, and may or may not be complementary to its partner nucleotide in the pair. Preferably it is complementary. Since the terminal nucleotide of the 5′ end of the second strand is paired with the terminal 3′ nucleotide of the first strand, the end of the scaffold polynucleotide to be extended is blunt-ended with no overhanging nucleotides. The terminal nucleotide of the 5′ end of the second strand comprises a phosphate group or any other suitable 5′ ligatable group, and therefore this terminal nucleotide is also a ligatable nucleotide.

The terminal ends of the scaffold polynucleotide which are not shown to be extended, i.e. those labelled 3′ and 5′ in FIG. 4, are preferably attached to a substrate, such as by any of the means described herein.

### Step 2—Ligation of a First Polynucleotide Ligation Molecule to the Scaffold Polynucleotide and Incorporation of One or More Nucleotides of the Predefined Sequence

In step (2) of the method a double-stranded polynucleotide ligation molecule is ligated (402) to the scaffold polynucleotide in a blunt-ended ligation reaction by the action of an enzyme having ligase activity.

The polynucleotide ligation molecule comprises a synthesis strand and a helper strand hybridised thereto. The polynucleotide ligation molecule further comprises a complementary ligation end comprising in the synthesis strand a universal nucleotide and a nucleotide of the predefined sequence.

The complementary ligation end of the first polynucleotide ligation molecule is structured such that the terminal nucleotide of the synthesis strand is the first nucleotide of the predefined sequence to be incorporated into the scaffold polynucleotide in any given cycle of synthesis. The terminal nucleotide of the synthesis strand is paired with the terminal nucleotide of the helper strand. In FIG. 4 the terminal nucleotide of the synthesis strand is depicted as “T” and the terminal nucleotide of the helper strand is depicted as “A”. Each of these designations are for illustrative purposes only. They can be any nucleotide, nucleotide analogue or nucleotide derivative, and they may or may not be complementary. Preferably they are complementary.

The universal nucleotide is the penultimate nucleotide at the terminal end of the synthesis strand at the complementary ligation end of the first polynucleotide ligation molecule. The universal nucleotide forms a nucleotide pair with the penultimate nucleotide at the terminal end of the helper strand at the complementary ligation end of the first polynucleotide ligation molecule. In FIG. 4 the penultimate nucleotide at the terminal end of the helper strand at the complementary ligation end is depicted as “X” for illustrative purposes only. It can be any nucleotide, nucleotide analogue or nucleotide derivative.

The terminal nucleotide of the synthesis strand at the complementary ligation end of the first polynucleotide ligation molecule is depicted in FIG. 4 at the 5′ end of the synthesis strand. This nucleotide is a ligatable nucleotide and is provided with a phosphate group or any other suitable 5′ ligatable group. The terminal nucleotide of the helper strand at the complementary ligation end of the first polynucleotide ligation molecule is depicted in FIG. 4 at the 3′ end of the helper strand. This nucleotide is provided as a non-ligatable nucleotide and comprises a non-ligatable 2′,3′-dideoxynucleotide or a 2′-deoxynucleotide, or any other suitable 3′ non-ligatable nucleotide.

The terminal nucleotide of the synthesis strand, i.e. the first nucleotide of the predefined sequence to be incorporated into the first strand in a given cycle of synthesis, occupies nucleotide position n in the synthesis strand. By position n in the synthesis strand of the first polynucleotide ligation molecule it is meant the position which will be occupied by the first nucleotide which is to be attached to the terminal end of the first strand of the scaffold polynucleotide following ligation of the polynucleotide ligation molecule to the scaffold polynucleotide. Position n also refers to the nucleotide position in the first strand of the ligated scaffold polynucleotide which is occupied by the said first nucleotide following its attachment to the terminal end of the first strand after ligation. Position n also refers to the nucleotide position in the second strand of the scaffold polynucleotide which will be occupied by a partner nucleotide for the said first nucleotide following attachment of the partner nucleotide to the terminal end of the second strand after the second extension/ligation reaction. Position n also refers to the nucleotide position which is occupied by the said partner nucleotide following its attachment to the terminal end of the second strand after the second extension/ligation reaction. The universal nucleotide in the synthesis strand of the first polynucleotide ligation molecule occupies position n+1.

The complementary ligation end of the first polynucleotide ligation molecule is configured so that it will compatibly join with the blunt end of the scaffold polynucleotide when subjected to suitable ligation conditions. Upon ligation of the synthesis strand of the polynucleotide ligation molecule and the first strand of the scaffold polynucleotide, the terminal nucleotide of the synthesis strand becomes incorporated into the first strand of the scaffold polynucleotide. Because the terminal nucleotide of the helper strand of the polynucleotide ligation molecule is a non-ligatable nucleotide, the ligase enzyme will be prevented from ligating the helper strand of the first polynucleotide ligation molecule and the second strand of the scaffold polynucleotide, thus creating a single-strand break or “nick” between the helper strand of the first polynucleotide ligation molecule and the second strand of the scaffold polynucleotide.

Ligation of the polynucleotide ligation molecule to the scaffold polynucleotide extends the length of the first strand of the double-stranded scaffold polynucleotide of step (1) and wherein the terminal nucleotide of the synthesis strand of the first polynucleotide ligation molecule is incorporated into the first strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

### Step 3—First Cleavage Step

In step (3) of the method the ligated scaffold polynucleotide is cleaved (403) at a cleavage site. The cleavage site is defined by a sequence comprising the universal nucleotide in the synthesis strand of the ligated first polynucleotide ligation molecule. Cleavage results in a double-strand break in the scaffold polynucleotide. Cleavage of the scaffold polynucleotide (step 3) results in loss of the helper strand of the ligated first polynucleotide ligation molecule and loss of the synthesis strand comprising the universal nucleotide. Cleavage of the scaffold polynucleotide thereby releases the polynucleotide ligation molecule from the scaffold polynucleotide but leads to the retention of the first nucleotide of that cycle attached to the first strand of the cleaved scaffold polynucleotide. Cleavage of the scaffold polynucleotide leaves in place a cleaved double-stranded scaffold polynucleotide comprising a singe-base overhang at the cleaved end. The first nucleotide of the predefined sequence occupies a position (n) as the terminal nucleotide of the first strand of the cleaved double-stranded scaffold polynucleotide and overhangs the terminal nucleotide of the second strand at the cleaved end.

The second strand of the ligated scaffold polynucleotide is already provided with a single-strand break or “nick” in this exemplary method, thus only cleavage of the first strand is necessary to provide a double-strand break in the scaffold polynucleotide. Furthermore, as noted previously, in this exemplary method version cleavage generates a cleaved double-stranded scaffold polynucleotide with a single-base overhang, with the terminal nucleotide of the first strand overhanging the terminal nucleotide of the second strand, and the universal nucleotide occupies position n+1 in the first strand prior to the cleavage step. To obtain such a cleaved double-stranded scaffold polynucleotide with a singe-base overhang when the universal nucleotide occupies position n+1 in the first strand, the ligated first strand is cleaved at a specific position relative to the universal nucleotide. When the first strand of the scaffold polynucleotide is cleaved between nucleotide positions n+1 and n the polynucleotide ligation molecule is released from the scaffold polynucleotide (see the structure depicted as exiting the synthesis cycle immediately after cleavage step 3 (403) in FIG. 4) except that the first nucleotide of that cycle derived from the first polynucleotide ligation molecule in step (2) is retained in the scaffold polynucleotide attached to the first strand of the cleaved scaffold polynucleotide.

A phosphate group, or any other suitable 5′ ligatable group, should continue to be attached to the terminal nucleotide of the second strand of the cleaved scaffold polynucleotide at the cleavage site. This ensures that the second strand of the cleaved scaffold polynucleotide can be ligated to the synthesis strand of the second polynucleotide ligation molecule in the second extension/ligation step (4). Cleavage is performed so that the terminal nucleotide of the first strand of the cleaved scaffold polynucleotide retains ligatable group, typically a hydroxyl group or any other suitable 3′ ligatable group, at the 3′ end of the first strand.

Thus in method version 4 the universal nucleotide occupies position n+1 in the synthesis/first strand at step (2) and the first strand is cleaved between nucleotide positions n+1 and n at step (3).

Preferably, the first strand is cleaved by cleavage of the phosphodiester bond between nucleotide positions n+1 and n (the first phosphodiester bond of the first strand relative to the position of the universal nucleotide, in the direction distal to the ligated polynucleotide ligation molecule/proximal to the first strand).

The first strand may be cleaved by cleavage of one ester bond of the phosphodiester bond between nucleotide positions n+1 and n.

Preferably the first strand is cleaved by cleavage of the first ester bond relative to nucleotide position n+1.

Any suitable mechanism may be employed to effect cleavage of the first strand between nucleotide positions n+1 and n when the universal nucleotide occupies position n+1.

Cleavage of the first strand between nucleotide positions n+1 and n as described above may be performed by the action of an enzyme.

Cleavage of the first strand between nucleotide positions n+1 and n as described above may be performed as a two-step cleavage process.

The first cleavage step of a two-step cleavage process may comprise removing the universal nucleotide from the first strand thus forming an abasic site at position n+1, and the second cleavage step may comprise cleaving the first strand at the abasic site, between positions n+1 and n.

One mechanism of cleaving the first strand at a cleavage site defined by a sequence comprising a universal nucleotide in the manner outlined above is described in analogous fashion in Example 2. The cleavage mechanism described in Example 2 is exemplary and other mechanisms could be employed, provided that the cleaved double-stranded scaffold polynucleotide described above is achieved.

In the first cleavage step of a two-step cleavage process the universal nucleotide is removed from the first strand whilst leaving the sugar-phosphate backbone intact. This can be achieved by the action of an enzyme which can specifically excise a single universal nucleotide from a double-stranded polynucleotide. In the exemplified cleavage methods the universal nucleotide is inosine and inosine is excised from the first strand by the action of an enzyme, thus forming an abasic site. In the exemplified cleavage method the enzyme is a 3-methyladenine DNA glycosylase enzyme, specifically human alkyladenine DNA glycosylase (hAAG). Other enzymes, molecules or chemicals could be used provided that an abasic site is formed. The nucleotide-excising enzyme may be an enzyme which catalyses the release of uracil from polynucleotides, such as Uracil-DNA Glycosylase (UDG).

In the second step of a two-step cleavage process the first strand is cleaved at the abasic site by making a single-strand break. In the exemplified methods the first strand is cleaved by the action of a chemical which is a base, such as NaOH. Alternatively, an organic chemical such as N,N′-dimethylethylenediamine may be used. Alternatively, enzymes having abasic site lyase activity, such as AP Endonuclease 1, Endonuclease III (Nth), or Endonuclease VIII, may be used. These enzymes cleave the DNA backbone at a phosphate group which is positioned 5′ relative to the abasic site. Cleavage thus exposes an OH group at the 3′ terminal end of the first strand which provides a terminal 3′ nucleotide which is ligatable in the first ligation step in the next cycle. Other enzymes, molecules or chemicals could be used provided that the first strand is cleaved at the abasic site as described.

Thus in embodiments wherein the universal nucleotide is at position n+1 of the first strand at step (2) and the first strand is cleaved between positions n+1 and n, a first cleavage step may be performed with a nucleotide-excising enzyme. An example of such an enzyme is a 3-methyladenine DNA glycosylase enzyme, such as human alkyladenine DNA glycosylase (hAAG). The second cleavage step may be performed with a chemical which is a base, such as NaOH. The second step may be performed with an organic chemical having abasic site cleavage activity such as N,N′-dimethylethylenediamine. The second step may be performed with an enzyme having abasic site lyase activity such as Endonuclease VIII or Endonuclease III.

Cleavage of the first strand between nucleotide positions n+1 and n as described above may also be performed as a one-step cleavage process. Examples of enzymes which may be used in any such process include Endonuclease III, Endonuclease VIII. Other enzymes which may be used in any such process include enzymes which cleave 8-oxoguanosine, such as formamidopyrimidine DNA glycosylase (Fpg) and 8-oxoguanine DNA glycosylase (hOGG1), which cleaves the DNA backbone so as to leave a phosphate group at the 3′ terminal end of the cleaved first strand, which can then be removed by Endonuclease IV or T4 polynucleotide kinase to expose an OH group which is ligatable in the first ligation step in the next cycle.

### Step 4—Ligation of a Second Polynucleotide Ligation Molecule to the Scaffold Polynucleotide and Incorporation of One or More Further Nucleotides of the Predefined Sequence

In step (4) of the method a second double-stranded polynucleotide ligation molecule is ligated (404) to the scaffold polynucleotide in a sticky—(complementary)—ended ligation reaction by the action of an enzyme having ligase activity.

The second polynucleotide ligation molecule comprises a synthesis strand and a helper strand hybridised thereto. The second polynucleotide ligation molecule further comprises a complementary ligation end comprising in the synthesis strand a universal nucleotide and one further nucleotide of the predefined sequence.

The complementary ligation end of the second polynucleotide ligation molecule is structured such that the terminal nucleotide of the synthesis strand is the first nucleotide of the predefined sequence to be incorporated into the second strand of the scaffold polynucleotide in any given cycle of synthesis.

The complementary ligation end comprises a single-base overhang. The terminal nucleotide of the synthesis strand is unpaired and overhangs the terminal nucleotide of the helper strand. The penultimate nucleotide of the synthesis strand is paired with the terminal nucleotide of the helper strand. In FIG. 4 the terminal nucleotide of the synthesis strand is depicted as “A”, the penultimate nucleotide of the synthesis strand is depicted as “X”, and the terminal nucleotide of the helper strand is depicted as “X”. Each of these designations are for illustrative purposes only. They can be any nucleotide, nucleotide analogue or nucleotide derivative. The penultimate nucleotide of the synthesis strand and the terminal nucleotide of the helper strand may or may not be complementary. Preferably they are complementary.

At the terminal end of the synthesis strand at the complementary ligation end of the second polynucleotide ligation molecule, the universal nucleotide occupies position which is the immediately next to the penultimate nucleotide of the synthesis strand in the direction distal to the complementary ligation end. The universal nucleotide occupies position n+2, the terminal nucleotide occupying position n and the penultimate nucleotide occupying position n+1. With reference to the definition of position n in the first extension/ligation reaction, position n refers to the nucleotide position in the second strand of the scaffold polynucleotide which is occupied by a partner nucleotide for the first nucleotide which is attached to the terminal end of the first strand after the first extension/ligation reaction.

The universal nucleotide forms a nucleotide pair with the nucleotide which is the penultimate nucleotide of the helper strand in the direction distal to the complementary ligation end. This is depicted in FIG. 4 as “X” for illustrative purposes only. It can be any nucleotide, nucleotide analogue or nucleotide derivative.

The terminal nucleotide of the synthesis strand at the complementary ligation end of the second polynucleotide ligation molecule is depicted in FIG. 4 at the 3′ end of the synthesis strand. This nucleotide is provided as a ligatable nucleotide and comprises a hydroxyl group or any other suitable 3′ ligatable group. The terminal nucleotide of the helper strand at the complementary ligation end of the second polynucleotide ligation molecule is depicted in FIG. 4 at the 5′ end of the helper strand. This nucleotide is provided as a non-ligatable nucleotide, e.g. lacking a phosphate group or provided with any suitable 5′ blocking group which can prevent ligation.

The complementary ligation end of the second polynucleotide ligation molecule is configured so that it will compatibly join with the overhanging end of the cleaved scaffold polynucleotide, generated in step (3), when subjected to suitable ligation conditions. Upon ligation of the synthesis strand of the second polynucleotide ligation molecule and the second strand of the cleaved scaffold polynucleotide, the terminal nucleotide of the synthesis strand of the second polynucleotide ligation becomes incorporated into the second strand of the cleaved scaffold polynucleotide. Because the terminal nucleotide of the helper strand of the second polynucleotide ligation molecule is a non-ligatable nucleotide, the ligase enzyme will be prevented from ligating the helper strand of the second polynucleotide ligation molecule and the second strand of the cleaved scaffold polynucleotide, thus creating a single-strand break or “nick” between the helper strand of the second polynucleotide ligation molecule and the second strand of the cleaved scaffold polynucleotide.

Ligation of the second polynucleotide ligation molecule to the cleaved scaffold polynucleotide extends the length of the second strand of the double-stranded scaffold polynucleotide of step (3) and wherein the terminal nucleotide of the synthesis strand of the second polynucleotide ligation molecule sequence is incorporated into the second strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

Upon ligation, the terminal nucleotide of the synthesis strand of the second polynucleotide ligation molecule pairs with the terminal nucleotide of the first strand of the cleaved scaffold polynucleotide generated in step (3), thus forming a nucleotide pair.

### Step 5—Second Cleavage Step

In step (5) of the method the ligated scaffold polynucleotide is cleaved (405) at a cleavage site. The cleavage site is defined by a sequence comprising the universal nucleotide in the synthesis strand of the ligated second polynucleotide ligation molecule. Cleavage results in a double-strand break in the scaffold polynucleotide. Cleavage of the scaffold polynucleotide (step 5) results in loss of the helper strand of the ligated second polynucleotide ligation molecule and loss of the synthesis strand comprising the universal nucleotide. Cleavage of the scaffold polynucleotide thereby releases the second polynucleotide ligation molecule from the scaffold polynucleotide but leads to the retention of the first nucleotide of that cycle attached to the second strand of the cleaved scaffold polynucleotide. Cleavage of the scaffold polynucleotide leaves in place a cleaved double-stranded scaffold polynucleotide comprising a blunt end at the cleaved end. The first nucleotide of the predefined sequence incorporated in step (4) occupies a position (n) as the terminal nucleotide of the second strand of the cleaved double-stranded scaffold polynucleotide, and the first nucleotide of the predefined sequence incorporated in step (2) occupies a position (also n) as the terminal nucleotide of the cleaved first strand. The first nucleotide of the predefined sequence incorporated into the second strand in step (4) and the first nucleotide of the predefined sequence incorporated into the first strand in step (4) are thus paired following the second extension/ligation and second cleavage step. In FIG. 4 these nucleotides are depicted (405) as “A” and “T” for illustrative purposes only. Each of these nucleotides can be any nucleotide, nucleotide analogue or nucleotide derivative and the pair may or may not be complementary. Preferably they are complementary.

Prior to the second cleavage step the first strand of the ligated scaffold polynucleotide is already provided with a single-strand break or “nick” in this exemplary method, thus only cleavage of the second strand is necessary to provide a double-strand break in the scaffold polynucleotide. Furthermore, as noted previously in this exemplary method version cleavage generates a cleaved double-stranded scaffold polynucleotide with a blunt end, and the universal nucleotide occupies position n+2 in the second strand prior to the cleavage step. To obtain such a cleaved double-stranded scaffold polynucleotide with a blunt end when the universal nucleotide occupies position n+2 in the second strand, the second strand is cleaved at a specific position relative to the universal nucleotide. When the second strand of the scaffold polynucleotide is cleaved between nucleotide positions n+1 and n the polynucleotide ligation molecule is released from the scaffold polynucleotide (see the structure depicted as exiting the synthesis cycle immediately after cleavage step 5 (505) in FIG. 5) except that the first nucleotide of that cycle derived from the second polynucleotide ligation molecule in step (4) is retained in the scaffold polynucleotide attached to the second strand of the cleaved scaffold polynucleotide.

A phosphate group, or any other suitable 5′ ligatable group, should continue to be attached to the terminal nucleotide of the second strand of the cleaved scaffold polynucleotide at the cleavage site. This ensures that the second strand of the cleaved scaffold polynucleotide can be ligated to the synthesis strand of the second polynucleotide ligation molecule in the second extension/ligation step (4) of the next cycle of synthesis. Cleavage is performed so that the terminal nucleotide of the first strand of the cleaved scaffold polynucleotide retains a ligatable group, typically a hydroxyl group or any other suitable 3′ ligatable group, at the 3′ end of the first strand.

Thus in method version 4 the universal nucleotide occupies position n+2 in the synthesis/second strand at step (4) and the second strand is cleaved between nucleotide positions n+1 and n at step (5).

Preferably, the second strand is cleaved by cleavage of the phosphodiester bond between nucleotide positions n+1 and n (the first phosphodiester bond of the second strand relative to the position of the universal nucleotide, in the direction distal to the ligated polynucleotide ligation molecule/proximal to the second strand).

The second strand may be cleaved by cleavage of one ester bond of the phosphodiester bond between nucleotide positions n+1 and n.

Preferably the second strand is cleaved by cleavage of the first ester bond relative to nucleotide position n+1.

Any suitable mechanism may be employed to effect cleavage of the second strand between nucleotide positions n+1 and n when the universal nucleotide occupies position n+2.

Cleavage of the second strand between nucleotide positions n+1 and n as described above may be performed by the action of an enzyme.

Cleavage of the second strand between nucleotide positions n+1 and n when the universal nucleotide occupies position n+2 in the second strand, as described above, may be performed by the action of an enzyme such as Endonuclease V.

One mechanism of cleaving the second strand between nucleotide positions n+1 and n at a cleavage site defined by a sequence comprising a universal nucleotide which is occupying position n+2 in the second strand is described in analogous fashion in Example 3. The mechanism described is exemplary and other mechanisms could be employed, provided that the cleavage arrangement described above is achieved.

In this exemplary mechanism an endonuclease enzyme is employed. In the exemplified method the enzyme is Endonuclease V. Other enzymes, molecules or chemicals could be used provided that the second strand is cleaved between nucleotide positions n+1 and n when the universal nucleotide occupies position n+2 in the second strand.

In synthesis method version 4 it will be noted that in any given cycle of synthesis, after the second cleavage step (step 5) the nucleotide positions which are occupied by the terminal nucleotide of the first and second strands at the cleaved end are both defined as nucleotide position n. These nucleotide positions are defined as nucleotide position n−1 in the next cycle of synthesis.

### Further Cycles

Following completion of the first cycle of synthesis, second and further cycles of synthesis may be performed using the same method steps.

The cleavage product of step (5) of the previous cycle is provided (in step 6) as the double-stranded scaffold polynucleotide for the next cycle of synthesis.

In step (7) of the next and each further cycle of synthesis a further first double-stranded polynucleotide ligation molecule is ligated to the cleavage product of step (5) of the previous cycle. The polynucleotide ligation molecule may be structured in the same way as described above for step (2) of the previous cycle, except that the further first polynucleotide ligation molecule comprises further first nd nucleotide of the further cycle of synthesis to be incorporated into the first strand. In step (7) the further first polynucleotide ligation molecule may be ligated to the cleavage product of step (5) of the previous cycle in the same way as described above for step (2).

In step (8) of the next and each further cycle of synthesis the ligated scaffold polynucleotide is subjected to a further first cleavage step at the cleavage site. Cleavage results in a double-strand break in the scaffold polynucleotide. Cleavage of the scaffold polynucleotide (step 8) results in loss of the helper strand and loss of the support strand comprising the universal nucleotide in the further first polynucleotide ligation molecule. Cleavage of the scaffold polynucleotide thereby releases the further first polynucleotide ligation molecule from the scaffold polynucleotide but leads to the retention of the further first nucleotide of that further cycle derived from the further first polynucleotide ligation molecule attached to the synthesis strand of the cleaved scaffold polynucleotide. Cleavage of the scaffold polynucleotide leaves in place a cleaved double-stranded scaffold polynucleotide comprising the further first nucleotide of the further cycle at the terminal end of the synthesis strand of the scaffold polynucleotide. Cleavage results in a single-base overhang with the terminal nucleotide of the first strand overhanging the terminal nucleotide of the second strand. Cleavage at step (8) may be performed in the same way as described above for step (4).

In step (9) of the next and each further cycle of synthesis a further second double-stranded polynucleotide ligation molecule is ligated to the cleavage product of step (8). The further second polynucleotide ligation molecule may be structured in the same way as described above for step (8) of the previous cycle, except that the further second polynucleotide ligation molecule comprises further first nucleotide of the further cycle of synthesis to be incorporated into the second strand. In step (9) the further second polynucleotide ligation molecule may be ligated to the cleavage product of step (8) in the same way as described above for step (4).

In step (10) of the next and each further cycle of synthesis the ligated scaffold polynucleotide is subjected to a further second cleavage step at the cleavage site. Cleavage results in a double-strand break in the scaffold polynucleotide. Cleavage of the scaffold polynucleotide (step 10) results in loss of the helper strand and loss of the support strand comprising the universal nucleotide in the further second polynucleotide ligation molecule. Cleavage of the scaffold polynucleotide thereby releases the further second polynucleotide ligation molecule from the scaffold polynucleotide but leads to the retention of the further first nucleotide of that further cycle derived from the further second polynucleotide ligation molecule attached to the synthesis strand of the cleaved scaffold polynucleotide. Cleavage of the scaffold polynucleotide leaves in place a cleaved blunt-ended double-stranded scaffold polynucleotide comprising the further first nucleotide of the further cycle derived from the further second polynucleotide ligation molecule at the terminal end of the second strand of the scaffold polynucleotide. Cleavage at step (10) may be performed in the same way as described above for step (5).

### Synthesis Method Version 5

### Step 1—Provision of a Scaffold Polynucleotide

In exemplary version 5 of the synthesis methods of the invention a double-stranded scaffold polynucleotide is provided in step (1) (501). The double-stranded scaffold polynucleotide is provided comprising a first strand and a second strand hybridised thereto. The terminal nucleotide of the first strand at the end to be extended is positioned at the 5′ end of the first strand and comprises a phosphate group or any other suitable 5′ ligatable group and therefore this terminal nucleotide is a ligatable nucleotide. In FIG. 5 this nucleotide is depicted as “X” and can be any nucleotide, nucleotide analogue or nucleotide derivative. The terminal nucleotide of the first strand at the end to be extended is depicted as paired with the terminal nucleotide of the 3′ end of the second strand. This terminal nucleotide is depicted as “X” and can be any nucleotide, nucleotide analogue or nucleotide derivative, and may or may not be complementary to its partner nucleotide in the pair. Preferably it is complementary. Since the terminal nucleotide of the 5′ end of the first strand is paired with the terminal 3′ nucleotide of the second strand, the end of the scaffold polynucleotide to be extended is blunt-ended with no overhanging nucleotides. The terminal nucleotide of the 3′ end of the second strand comprises a hydroxyl group or any other suitable 3′ ligatable group, and therefore this terminal nucleotide is also a ligatable nucleotide.

The terminal ends of the scaffold polynucleotide which are not shown to be extended, i.e. those labelled 3′ and 5′ in FIG. 5, are preferably attached to a substrate, such as by any of the means described herein.

### Step 2—Ligation of a First Polynucleotide Ligation Molecule to the Scaffold Polynucleotide and Incorporation of One or More Nucleotides of the Predefined Sequence

In step (2) of the method a double-stranded polynucleotide ligation molecule is ligated (502) to the scaffold polynucleotide in a blunt-ended ligation reaction by the action of an enzyme having ligase activity.

The polynucleotide ligation molecule comprises a synthesis strand and a helper strand hybridised thereto. The polynucleotide ligation molecule further comprises a complementary ligation end comprising in the synthesis strand a universal nucleotide and a nucleotide of the predefined sequence.

The complementary ligation end of the first polynucleotide ligation molecule is structured such that the terminal nucleotide of the synthesis strand is the first nucleotide of the predefined sequence to be incorporated into the scaffold polynucleotide in any given cycle of synthesis. The terminal nucleotide of the synthesis strand is paired with the terminal nucleotide of the helper strand. In FIG. 5 the terminal nucleotide of the synthesis strand is depicted as “T” and the terminal nucleotide of the helper strand is depicted as “A”. Each of these designations are for illustrative purposes only. They can be any nucleotide, nucleotide analogue or nucleotide derivative, and they may or may not be complementary. Preferably they are complementary.

The universal nucleotide is the penultimate nucleotide at the terminal end of the synthesis strand at the complementary ligation end of the first polynucleotide ligation molecule. The universal nucleotide forms a nucleotide pair with the penultimate nucleotide at the terminal end of the helper strand at the complementary ligation end of the first polynucleotide ligation molecule. In FIG. 5 the penultimate nucleotide at the terminal end of the helper strand at the complementary ligation end is depicted as “X” for illustrative purposes only. It can be any nucleotide, nucleotide analogue or nucleotide derivative.

The terminal nucleotide of the synthesis strand at the complementary ligation end of the first polynucleotide ligation molecule is depicted in FIG. 5 at the 3′ end of the synthesis strand. This nucleotide is provided as a ligatable nucleotide and comprises a hydroxyl group, or any other suitable 3′ ligatable group. The terminal nucleotide of the helper strand at the complementary ligation end of the first polynucleotide ligation molecule is depicted in FIG. 5 at the 5′ end of the helper strand. This nucleotide is provided as a non-ligatable nucleotide and is provided without a phosphate group or with any other suitable 5′ non-ligatable or blocking group.

The terminal nucleotide of the synthesis strand, i.e. the first nucleotide of the predefined sequence to be incorporated into the first strand in a given cycle of synthesis, occupies nucleotide position n in the synthesis strand. By position n in the synthesis strand of the first polynucleotide ligation molecule it is meant the position which will be occupied by the first nucleotide which is to be attached to the terminal end of the first strand of the scaffold polynucleotide following ligation of the polynucleotide ligation molecule to the scaffold polynucleotide. Position n also refers to the nucleotide position in the first strand of the ligated scaffold polynucleotide which is occupied by the said first nucleotide following its attachment to the terminal end of the first strand after ligation. Position n also refers to the nucleotide position in the second strand of the scaffold polynucleotide which will be occupied by a partner nucleotide for the said first nucleotide following attachment of the partner nucleotide to the terminal end of the second strand after the second extension/ligation reaction. Position n also refers to the nucleotide position which is occupied by the said partner nucleotide following its attachment to the terminal end of the second strand after the second extension/ligation reaction. The universal nucleotide in the synthesis strand of the first polynucleotide ligation molecule occupies position n+1.

The complementary ligation end of the first polynucleotide ligation molecule is configured so that it will compatibly join with the blunt end of the scaffold polynucleotide when subjected to suitable ligation conditions. Upon ligation of the synthesis strand of the polynucleotide ligation molecule and the first strand of the scaffold polynucleotide, the terminal nucleotide of the synthesis strand becomes incorporated into the first strand of the scaffold polynucleotide. Because the terminal nucleotide of the helper strand of the polynucleotide ligation molecule is a non-ligatable nucleotide, the ligase enzyme will be prevented from ligating the helper strand of the first polynucleotide ligation molecule and the second strand of the scaffold polynucleotide, thus creating a single-strand break or “nick” between the helper strand of the first polynucleotide ligation molecule and the second strand of the scaffold polynucleotide.

Ligation of the polynucleotide ligation molecule to the scaffold polynucleotide extends the length of the first strand of the double-stranded scaffold polynucleotide of step (1) and wherein the terminal nucleotide of the synthesis strand of the first polynucleotide ligation molecule is incorporated into the first strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

### Step 3—First Cleavage Step

In step (3) of the method the ligated scaffold polynucleotide is cleaved (503) at a cleavage site. The cleavage site is defined by a sequence comprising the universal nucleotide in the synthesis strand of the ligated first polynucleotide ligation molecule. Cleavage results in a double-strand break in the scaffold polynucleotide. Cleavage of the scaffold polynucleotide (step 3) results in loss of the helper strand of the ligated first polynucleotide ligation molecule and loss of the synthesis strand comprising the universal nucleotide. Cleavage of the scaffold polynucleotide thereby releases the polynucleotide ligation molecule from the scaffold polynucleotide but leads to the retention of the first nucleotide of that cycle attached to the first strand of the cleaved scaffold polynucleotide. Cleavage of the scaffold polynucleotide leaves in place a cleaved double-stranded scaffold polynucleotide comprising a singe-base overhang at the cleaved end. The first nucleotide of the predefined sequence occupies a position (n) as the terminal nucleotide of the first strand of the cleaved double-stranded scaffold polynucleotide and overhangs the terminal nucleotide of the second strand at the cleaved end.

The second strand of the ligated scaffold polynucleotide is already provided with a single-strand break or “nick” in this exemplary method, thus only cleavage of the first strand is necessary to provide a double-strand break in the scaffold polynucleotide. Furthermore, as noted previously, in this exemplary method version cleavage generates a cleaved double-stranded scaffold polynucleotide with a single-base overhang, with the terminal nucleotide of the first strand overhanging the terminal nucleotide of the second strand, and the universal nucleotide occupies position n+1 in the first strand prior to the cleavage step. To obtain such a cleaved double-stranded scaffold polynucleotide with a singe-base overhang when the universal nucleotide occupies position n+1 in the first strand, the ligated first strand is cleaved at a specific position relative to the universal nucleotide. When the first strand of the scaffold polynucleotide is cleaved between nucleotide positions n+1 and n the polynucleotide ligation molecule is released from the scaffold polynucleotide (see the structure depicted as exiting the synthesis cycle immediately after cleavage step 3 (503) in FIG. 5) except that the first nucleotide of that cycle derived from the first polynucleotide ligation molecule in step (2) is retained in the scaffold polynucleotide attached to the first strand of the cleaved scaffold polynucleotide.

A hydroxyl group, or any other suitable 3′ ligatable group, should continue to be attached to the terminal nucleotide of the second strand of the cleaved scaffold polynucleotide at the cleavage site. This ensures that the second strand of the cleaved scaffold polynucleotide can be ligated to the synthesis strand of the second polynucleotide ligation molecule in the second extension/ligation step (4). Cleavage is performed so that the terminal nucleotide of the first strand of the cleaved scaffold polynucleotide retains a ligatable group, typically a phosphate group or any other suitable 5′ ligatable group, at the 5′ end of the first strand.

Thus in method version 5 the universal nucleotide occupies position n+1 in the synthesis/first strand at step (2) and the first strand is cleaved between nucleotide positions n+1 and n at step (3).

Preferably, the first strand is cleaved by cleavage of the phosphodiester bond between nucleotide positions n+1 and n (the first phosphodiester bond of the first strand relative to the position of the universal nucleotide, in the direction distal to the ligated polynucleotide ligation molecule/proximal to the first strand).

The first strand may be cleaved by cleavage of one ester bond of the phosphodiester bond between nucleotide positions n+1 and n.

Preferably the first strand is cleaved by cleavage of the first ester bond relative to nucleotide position n+1.

Any suitable mechanism may be employed to effect cleavage of the first strand between nucleotide positions n+1 and n when the universal nucleotide occupies position n+1.

Cleavage of the first strand between nucleotide positions n+1 and n as described above may be performed by the action of an enzyme.

Cleavage of the first strand between nucleotide positions n+1 and n as described above may be performed as a two-step cleavage process.

The first cleavage step of a two-step cleavage process may comprise removing the universal nucleotide from the first strand thus forming an abasic site at position n+1, and the second cleavage step may comprise cleaving the first strand at the abasic site, between positions n+1 and n.

One mechanism of cleaving the first strand at a cleavage site defined by a sequence comprising a universal nucleotide in the manner outlined above is described in analogous fashion in Example 2. The cleavage mechanism described in Example 2 is exemplary and other mechanisms could be employed, provided that the cleaved double-stranded scaffold polynucleotide described above is achieved.

In the first cleavage step of a two-step cleavage process the universal nucleotide is removed from the first strand whilst leaving the sugar-phosphate backbone intact. This can be achieved by the action of an enzyme which can specifically excise a single universal nucleotide from a double-stranded polynucleotide. In the exemplified cleavage methods the universal nucleotide is inosine and inosine is excised from the first strand by the action of an enzyme, thus forming an abasic site. In the exemplified cleavage method the enzyme is a 3-methyladenine DNA glycosylase enzyme, specifically human alkyladenine DNA glycosylase (hAAG). Other enzymes, molecules or chemicals could be used provided that an abasic site is formed. The nucleotide-excising enzyme may be an enzyme which catalyses the release of uracil from polynucleotides, such as Uracil-DNA Glycosylase (UDG).

In the second step of a two-step cleavage process the first strand is cleaved at the abasic site by making a single-strand break. In the exemplified methods the first strand is cleaved by the action of a chemical which is a base, such as NaOH. Alternatively, an organic chemical such as N,N′-dimethylethylenediamine may be used. Alternatively, enzymes having abasic site lyase activity, such as AP Endonuclease 1, Endonuclease III (Nth), or Endonuclease VIII, may be used. Other enzymes, molecules or chemicals could be used provided that the first strand is cleaved at the abasic site as described.

Thus in embodiments wherein the universal nucleotide is at position n+1 of the first strand at step (2) and the first strand is cleaved between positions n+1 and n, a first cleavage step may be performed with a nucleotide-excising enzyme. An example of such an enzyme is a 3-methyladenine DNA glycosylase enzyme, such as human alkyladenine DNA glycosylase (hAAG). The second cleavage step may be performed with a chemical which is a base, such as NaOH. The second step may be performed with an organic chemical having abasic site cleavage activity such as N,N′-dimethylethylenediamine. The second step may be performed with an enzyme having abasic site lyase activity such as Endonuclease VIII or Endonuclease III.

Cleavage of the first strand between nucleotide positions n+1 and n as described above may also be performed as a one-step cleavage process. Examples of enzymes which may be used in any such process include Endonuclease III, Endonuclease VIII. Other enzymes which may be used in any such process include enzymes which cleave 8-oxoguanosine, such as formamidopyrimidine DNA glycosylase (Fpg) and 8-oxoguanine DNA glycosylase (hOGG1).

### Step 4—Ligation of a Second Polynucleotide Ligation Molecule to the Scaffold Polynucleotide and Incorporation of One or More Further Nucleotides of the Predefined Sequence

In step (4) of the method a second double-stranded polynucleotide ligation molecule is ligated (504) to the cleaved scaffold polynucleotide in a sticky-(complementary)-ended ligation reaction by the action of an enzyme having ligase activity.

The second polynucleotide ligation molecule comprises a synthesis strand and a helper strand hybridised thereto. The second polynucleotide ligation molecule further comprises a complementary ligation end comprising in the synthesis strand a universal nucleotide and a further nucleotide of the predefined sequence.

The complementary ligation end of the second polynucleotide ligation molecule is structured such that the terminal nucleotide of the synthesis strand is the first nucleotide of the predefined sequence to be incorporated into the second strand of the cleaved scaffold polynucleotide in any given cycle of synthesis.

The complementary ligation end comprises a single-base overhang. The terminal nucleotide of the synthesis strand is unpaired and overhangs the terminal nucleotide of the helper strand. In FIG. 5 the terminal nucleotide of the synthesis strand is depicted as “A” for illustrative purposes only. It can be any nucleotide, nucleotide analogue or nucleotide derivative.

The universal nucleotide is the penultimate nucleotide at the terminal end of the synthesis strand at the complementary ligation end of the second polynucleotide ligation molecule. The universal nucleotide forms a nucleotide pair with the terminal nucleotide of the helper strand at the complementary ligation end of the second polynucleotide ligation molecule. In FIG. 5 the terminal nucleotide of the helper strand at the complementary ligation end is depicted as “X” for illustrative purposes only. It can be any nucleotide, nucleotide analogue or nucleotide derivative.

The terminal nucleotide of the synthesis strand at the complementary ligation end of the second polynucleotide ligation molecule is depicted in FIG. 5 at the 5′ end of the synthesis strand. This nucleotide is a ligatable nucleotide and is provided with a phosphate group or any other suitable 5′ ligatable group. The terminal nucleotide of the helper strand at the complementary ligation end of the second polynucleotide ligation molecule is depicted in FIG. 5 at the 3′ end of the helper strand. This nucleotide is provided as a non-ligatable nucleotide and comprises a non-ligatable 2′,3′-dideoxynucleotide or a 2′-deoxynucleotide, or any other suitable 3′ non-ligatable nucleotide.

The terminal nucleotide of the synthesis strand, i.e. the first nucleotide of the predefined sequence of that cycle to be incorporated into the second strand of the cleaved scaffold polynucleotide, occupies nucleotide position n in the synthesis strand. With reference to the definition of position n in the first extension/ligation reaction, position n refers to the nucleotide position in the synthesis strand of the second polynucleotide ligation molecule which is occupied by a nucleotide which will be a partner nucleotide for the first nucleotide of step (2) in the first strand following attachment of the partner nucleotide to the terminal end of the second strand after the second extension/ligation reaction. Position n also refers to the nucleotide position which is occupied by the said partner nucleotide following its attachment to the terminal end of the second strand after the second extension/ligation reaction. Position n also refers to the nucleotide position in the first strand of the scaffold polynucleotide which is occupied by the first nucleotide following its attachment to the terminal end of the first strand after ligation in step (2).

The universal nucleotide in the synthesis strand of the second polynucleotide ligation molecule occupies position n+1.

The complementary ligation end of the second polynucleotide ligation molecule is configured so that it will compatibly join with the overhanging end of the cleaved scaffold polynucleotide, generated in step (3), when subjected to suitable ligation conditions. Upon ligation of the synthesis strand of the second polynucleotide ligation molecule and the second strand of the scaffold polynucleotide, the terminal nucleotide of the synthesis strand of the second polynucleotide ligation becomes incorporated into the second strand of the cleaved scaffold polynucleotide. Because the terminal nucleotide of the helper strand of the second polynucleotide ligation molecule is a non-ligatable nucleotide, the ligase enzyme will be prevented from ligating the helper strand of the second polynucleotide ligation molecule and the first strand of the cleaved scaffold polynucleotide, thus creating a single-strand break or “nick” between the helper strand of the second polynucleotide ligation molecule and the first strand of the cleaved scaffold polynucleotide.

Ligation of the second polynucleotide ligation molecule to the cleaved scaffold polynucleotide extends the length of the second strand of the double-stranded scaffold polynucleotide of step (3) and wherein the terminal nucleotide of the synthesis strand of the second polynucleotide ligation molecule sequence is incorporated into the second strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

Upon ligation, the terminal nucleotide of the synthesis strand of the second polynucleotide ligation molecule pairs with the terminal nucleotide of the first strand of the cleaved scaffold polynucleotide generated in step (3), thus forming a nucleotide pair.

### Step 5—Second Cleavage Step

In step (5) of the method the ligated scaffold polynucleotide is cleaved (505) at a cleavage site. The cleavage site is defined by a sequence comprising the universal nucleotide in the synthesis strand of the ligated second polynucleotide ligation molecule. Cleavage results in a double-strand break in the scaffold polynucleotide. Cleavage of the scaffold polynucleotide (step 5) results in loss of the helper strand of the ligated second polynucleotide ligation molecule and loss of the synthesis strand comprising the universal nucleotide. Cleavage of the scaffold polynucleotide thereby releases the second polynucleotide ligation molecule from the scaffold polynucleotide but leads to the retention of the second nucleotide of that cycle attached to the second strand of the cleaved scaffold polynucleotide. Cleavage of the scaffold polynucleotide leaves in place a cleaved double-stranded scaffold polynucleotide comprising a blunt end. The second nucleotide of the predefined sequence incorporated in step (4) occupies a position (n) as the terminal nucleotide of the second strand of the cleaved double-stranded scaffold polynucleotide and is paired with the first/terminal nucleotide of the first strand of the cleaved double-stranded scaffold polynucleotide which was incorporated in step (2) and which occupies position n in the first strand. In FIG. 5 these nucleotides are depicted (505) as “A” and “T” for illustrative purposes only. Each of these nucleotides can be any nucleotide, nucleotide analogue or nucleotide derivative and the pair may or may not be complementary. Preferably they are complementary.

Prior to the second cleavage step the first strand of the ligated scaffold polynucleotide is already provided with a single-strand break or “nick” in this exemplary method, thus only cleavage of the second strand is necessary to provide a double-strand break in the scaffold polynucleotide. Furthermore, as noted previously in this exemplary method version cleavage generates a cleaved double-stranded scaffold polynucleotide with a blunt end, and the universal nucleotide occupies position n+1 in the second strand prior to the second cleavage step. To obtain such a cleaved double-stranded scaffold polynucleotide with a blunt end when the universal nucleotide occupies position n+1 in the second strand, the second strand is cleaved at a specific position relative to the universal nucleotide. When the second strand of the scaffold polynucleotide is cleaved between nucleotide positions n+1 and n the polynucleotide ligation molecule is released from the scaffold polynucleotide (see the structure depicted as exiting the synthesis cycle immediately after cleavage step 5 (505) in FIG. 5) except that the first nucleotide of that cycle derived from the second polynucleotide ligation molecule in step (4) is retained in the scaffold polynucleotide attached to the second strand of the cleaved scaffold polynucleotide.

A phosphate group, or any other suitable 5′ ligatable group, should continue to be attached to the terminal nucleotide of the first strand of the cleaved scaffold polynucleotide at the cleavage site. This ensures that the first strand of the cleaved scaffold polynucleotide can be ligated to the synthesis strand of the first polynucleotide ligation molecule in the first extension/ligation step (2) of the next cycle of synthesis. Cleavage is performed so that the terminal nucleotide of the second strand of the cleaved scaffold polynucleotide retains a ligatable group, typically a hydroxyl group or any other suitable 3′ ligatable group, at the 3′ end of the first strand.

Thus in method version 5 the universal nucleotide occupies position n+1 in the synthesis/second strand at step (4) and the second strand is cleaved between nucleotide positions n+1 and n at step (5).

Preferably, the second strand is cleaved by cleavage of the phosphodiester bond between nucleotide positions n+1 and n (the first phosphodiester bond of the second strand relative to the position of the universal nucleotide, in the direction distal to the ligated polynucleotide ligation molecule/proximal to the second strand).

The second strand may be cleaved by cleavage of one ester bond of the phosphodiester bond between nucleotide positions n+1 and n.

Preferably the second strand is cleaved by cleavage of the first ester bond relative to nucleotide position n+1.

Any suitable mechanism may be employed to effect cleavage of the second strand between nucleotide positions n+1 and n when the universal nucleotide occupies position n+1.

Cleavage of the second strand between nucleotide positions n+1 and n as described above may be performed by the action of an enzyme.

Cleavage of the second strand between nucleotide positions n+1 and n as described above may be performed as a two-step cleavage process.

The first cleavage step of a two-step cleavage process may comprise removing the universal nucleotide from the second strand thus forming an abasic site at position n+1, and the second cleavage step may comprise cleaving the second strand at the abasic site, between positions n+1 and n.

One mechanism of cleaving the second strand at a cleavage site defined by a sequence comprising a universal nucleotide in the manner outlined above is described in analogous fashion in Example 2. The cleavage mechanism described in Example 2 is exemplary and other mechanisms could be employed, provided that the cleaved double-stranded scaffold polynucleotide described above is achieved.

In the first cleavage step of a two-step cleavage process the universal nucleotide is removed from the second strand whilst leaving the sugar-phosphate backbone intact. This can be achieved by the action of an enzyme which can specifically excise a single universal nucleotide from a double-stranded polynucleotide. In the exemplified cleavage methods the universal nucleotide is inosine and inosine is excised from the strand by the action of an enzyme, thus forming an abasic site. In the exemplified cleavage method the enzyme is a 3-methyladenine DNA glycosylase enzyme, specifically human alkyladenine DNA glycosylase (hAAG). Other enzymes, molecules or chemicals could be used provided that an abasic site is formed. The nucleotide-excising enzyme may be an enzyme which catalyses the release of uracil from polynucleotides, such as Uracil-DNA Glycosylase (UDG).

In the second step of a two-step cleavage process the second strand is cleaved at the abasic site by making a single-strand break. In the exemplified methods the strand is cleaved by the action of a chemical which is a base, such as NaOH. Alternatively, an organic chemical such as N,N′-dimethylethylenediamine may be used. Alternatively, enzymes having abasic site lyase activity, such as AP Endonuclease 1, Endonuclease III (Nth), or Endonuclease VIII, may be used. These enzymes cleave the DNA backbone at a phosphate group which is positioned 5′ relative to the abasic site. Cleavage thus exposes an OH group at the 3′ terminal end of the second strand which provides a terminal 3′ nucleotide which is ligatable in the second ligation step in the next cycle. Other enzymes, molecules or chemicals could be used provided that the second strand is cleaved at the abasic site as described.

Thus in embodiments wherein the universal nucleotide is at position n+1 of the second strand at step (4) and the second strand is cleaved between positions n+1 and n, a first cleavage step may be performed with a nucleotide-excising enzyme. An example of such an enzyme is a 3-methyladenine DNA glycosylase enzyme, such as human alkyladenine DNA glycosylase (hAAG). The second cleavage step may be performed with a chemical which is a base, such as NaOH. The second step may be performed with an organic chemical having abasic site cleavage activity such as N,N′-dimethylethylenediamine. The second step may be performed with an enzyme having abasic site lyase activity such as Endonuclease VIII or Endonuclease III.

Cleavage of the second strand between nucleotide positions n+1 and n as described above may also be performed as a one-step cleavage process. Examples of enzymes which may be used in any such process include Endonuclease III, Endonuclease VIII. Other enzymes which may be used in any such process include enzymes which cleave 8-oxoguanosine, such as formamidopyrimidine DNA glycosylase (Fpg) and 8-oxoguanine DNA glycosylase (hOGG1), which cleaves the DNA backbone so as to leave a phosphate group at the 3′ terminal end of the cleaved second strand, which can then be removed by Endonuclease IV or T4 polynucleotide kinase to expose an OH group which is ligatable in the second ligation step in the next cycle.

In synthesis method version 5 it will be noted that in any given cycle of synthesis, after the second cleavage step (step 5) the nucleotide positions which are occupied by the terminal nucleotide of the first and second strands at the cleaved end are both defined as nucleotide position n. These nucleotide positions are defined as nucleotide position n−1 in the next cycle of synthesis.

### Further Cycles

Following completion of the first cycle of synthesis, second and further cycles of synthesis may be performed using the same method steps.

The cleavage product of step (5) of the previous cycle is provided (in step 6) as the double-stranded scaffold polynucleotide for the next cycle of synthesis.

In step (7) of the next and each further cycle of synthesis a further first double-stranded polynucleotide ligation molecule is ligated to the cleavage product of step (5) of the previous cycle. The polynucleotide ligation molecule may be structured in the same way as described above for step (2) of the previous cycle, except that the further first polynucleotide ligation molecule comprises further first nd nucleotide of the further cycle of synthesis to be incorporated into the first strand. In step (7) the further first polynucleotide ligation molecule may be ligated to the cleavage product of step (5) of the previous cycle in the same way as described above for step (2).

In step (8) of the next and each further cycle of synthesis the ligated scaffold polynucleotide is subjected to a further first cleavage step at the cleavage site. Cleavage results in a double-strand break in the scaffold polynucleotide. Cleavage of the scaffold polynucleotide (step 8) results in loss of the helper strand and loss of the support strand comprising the universal nucleotide in the further first polynucleotide ligation molecule. Cleavage of the scaffold polynucleotide thereby releases the further first polynucleotide ligation molecule from the scaffold polynucleotide but leads to the retention of the further first nucleotide of that further cycle derived from the further first polynucleotide ligation molecule attached to the synthesis strand of the cleaved scaffold polynucleotide. Cleavage of the scaffold polynucleotide leaves in place a cleaved double-stranded scaffold polynucleotide comprising the further first nucleotide of the further cycle at the terminal end of the synthesis strand of the scaffold polynucleotide. Cleavage results in a single-base overhang with the terminal nucleotide of the first strand overhanging the terminal nucleotide of the second strand. Cleavage at step (8) may be performed in the same way as described above for step (4).

In step (9) of the next and each further cycle of synthesis a further second double-stranded polynucleotide ligation molecule is ligated to the cleavage product of step (8). The further second polynucleotide ligation molecule may be structured in the same way as described above for step (8) of the previous cycle, except that the further second polynucleotide ligation molecule comprises further first nucleotide of the further cycle of synthesis to be incorporated into the second strand. In step (9) the further second polynucleotide ligation molecule may be ligated to the cleavage product of step (8) in the same way as described above for step (4).

In step (10) of the next and each further cycle of synthesis the ligated scaffold polynucleotide is subjected to a further second cleavage step at the cleavage site. Cleavage results in a double-strand break in the scaffold polynucleotide. Cleavage of the scaffold polynucleotide (step 10) results in loss of the helper strand and loss of the support strand comprising the universal nucleotide in the further second polynucleotide ligation molecule. Cleavage of the scaffold polynucleotide thereby releases the further second polynucleotide ligation molecule from the scaffold polynucleotide but leads to the retention of the further first nucleotide of that further cycle derived from the further second polynucleotide ligation molecule attached to the synthesis strand of the cleaved scaffold polynucleotide. Cleavage of the scaffold polynucleotide leaves in place a cleaved blunt-ended double-stranded scaffold polynucleotide comprising the further first nucleotide of the further cycle derived from the further second polynucleotide ligation molecule at the terminal end of the second strand of the scaffold polynucleotide. Cleavage at step (10) may be performed in the same way as described above for step (5).

### Synthesis Method Version 6

### Step 1—Provision of a Scaffold Polynucleotide

In exemplary version 6 of the synthesis methods of the invention a double-stranded scaffold polynucleotide is provided in step (1) (601). The double-stranded scaffold polynucleotide is provided comprising a first strand and a second strand hybridised thereto. The terminal nucleotide of the first strand at the end to be extended is positioned at the 5′ end of the first strand and comprises a phosphate group or any other suitable 5′ ligatable group and therefore this terminal nucleotide is a ligatable nucleotide. In FIG. 1 this nucleotide is depicted as “X” and can be any nucleotide, nucleotide analogue or nucleotide derivative. The terminal nucleotide of the first strand at the end to be extended is depicted as paired with the terminal nucleotide of the 3′ end of the second strand. This terminal nucleotide is depicted as “X” and can be any nucleotide, nucleotide analogue or nucleotide derivative, and may or may not be complementary to its partner nucleotide in the pair. Preferably it is complementary. Since the terminal nucleotide of the 5′ end of the first strand is paired with the terminal 3′ nucleotide of the second strand, the end of the scaffold polynucleotide to be extended is blunt-ended with no overhanging nucleotides. The terminal nucleotide of the 3′ end of the second strand comprises a hydroxyl group or any other suitable 3′ ligatable group, and therefore this terminal nucleotide is also a ligatable nucleotide.

The terminal ends of the scaffold polynucleotide which are not shown to be extended, i.e. those labelled 3′ and 5′ in FIG. 6, are preferably attached to a substrate, such as by any of the means described herein.

### Step 2—Ligation of a First Polynucleotide Ligation Molecule to the Scaffold Polynucleotide and Incorporation of One or More Nucleotides of the Predefined Sequence

In step (2) of the method a double-stranded polynucleotide ligation molecule is ligated (602) to the scaffold polynucleotide in a blunt-ended ligation reaction by the action of an enzyme having ligase activity.

The polynucleotide ligation molecule comprises a synthesis strand and a helper strand hybridised thereto. The polynucleotide ligation molecule further comprises a complementary ligation end comprising in the synthesis strand a universal nucleotide and a nucleotide of the predefined sequence.

The complementary ligation end of the first polynucleotide ligation molecule is structured such that the terminal nucleotide of the synthesis strand is the first nucleotide of the predefined sequence to be incorporated into the scaffold polynucleotide in any given cycle of synthesis. The terminal nucleotide of the synthesis strand is paired with the terminal nucleotide of the helper strand. In FIG. 6 the terminal nucleotide of the synthesis strand is depicted as “T” and the terminal nucleotide of the helper strand is depicted as “A”. Each of these designations are for illustrative purposes only. They can be any nucleotide, nucleotide analogue or nucleotide derivative, and they may or may not be complementary. Preferably they are complementary.

At the terminal end of the synthesis strand at the complementary ligation end of the first polynucleotide ligation molecule the universal nucleotide occupies the position immediately next to the penultimate nucleotide in the direction distal to the complementary ligation end. The universal nucleotide forms a nucleotide pair with the nucleotide which occupies the position immediately next to the penultimate nucleotide in the helper strand in the direction distal to the complementary ligation end. In FIG. 6 the penultimate nucleotide at the terminal ends of the synthesis and helper strands at the complementary ligation end are depicted as “X” for illustrative purposes only. Each of these nucleotides can be any nucleotide, nucleotide analogue or nucleotide derivative, and they may or may not be complementary. Preferably they are complementary.

The terminal nucleotide of the synthesis strand at the complementary ligation end of the first polynucleotide ligation molecule is depicted in FIG. 6 at the 3′ end of the synthesis strand. This nucleotide is provided as a ligatable nucleotide and comprises a hydroxyl group, or any other suitable 3′ ligatable group. The terminal nucleotide of the helper strand at the complementary ligation end of the first polynucleotide ligation molecule is depicted in FIG. 6 at the 5′ end of the helper strand. This nucleotide is provided as a non-ligatable nucleotide and is provided without a phosphate group or with any other suitable 5′ non-ligatable or blocking group.

The terminal nucleotide of the synthesis strand, i.e. the first nucleotide of the predefined sequence to be incorporated into the first strand in a given cycle of synthesis, occupies nucleotide position n in the synthesis strand. By position n in the synthesis strand of the first polynucleotide ligation molecule it is meant the position which will be occupied by the first nucleotide which is to be attached to the terminal end of the first strand of the scaffold polynucleotide following ligation of the polynucleotide ligation molecule to the scaffold polynucleotide. Position n also refers to the nucleotide position in the first strand of the ligated scaffold polynucleotide which is occupied by the said first nucleotide following its attachment to the terminal end of the first strand after ligation. Position n also refers to the nucleotide position in the second strand of the scaffold polynucleotide which will be occupied by a partner nucleotide for the said first nucleotide following attachment of the partner nucleotide to the terminal end of the second strand after the second extension/ligation reaction. Position n also refers to the nucleotide position which is occupied by the said partner nucleotide following its attachment to the terminal end of the second strand after the second extension/ligation reaction. The universal nucleotide in the synthesis strand of the first polynucleotide ligation molecule occupies position n+2.

The complementary ligation end of the first polynucleotide ligation molecule is configured so that it will compatibly join with the blunt end of the scaffold polynucleotide when subjected to suitable ligation conditions. Upon ligation of the synthesis strand of the polynucleotide ligation molecule and the first strand of the scaffold polynucleotide, the terminal nucleotide of the synthesis strand becomes incorporated into the first strand of the scaffold polynucleotide. Because the terminal nucleotide of the helper strand of the polynucleotide ligation molecule is a non-ligatable nucleotide, the ligase enzyme will be prevented from ligating the helper strand of the first polynucleotide ligation molecule and the second strand of the scaffold polynucleotide, thus creating a single-strand break or “nick” between the helper strand of the first polynucleotide ligation molecule and the second strand of the scaffold polynucleotide.

Ligation of the polynucleotide ligation molecule to the scaffold polynucleotide extends the length of the first strand of the double-stranded scaffold polynucleotide of step (1) and wherein the terminal nucleotide of the synthesis strand of the first polynucleotide ligation molecule is incorporated into the first strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

### Step 3—First Cleavage Step

In step (3) of the method the ligated scaffold polynucleotide is cleaved (603) at a cleavage site. The cleavage site is defined by a sequence comprising the universal nucleotide in the synthesis strand of the ligated first polynucleotide ligation molecule. Cleavage results in a double-strand break in the scaffold polynucleotide. Cleavage of the scaffold polynucleotide (step 3) results in loss of the helper strand of the ligated first polynucleotide ligation molecule and loss of the synthesis strand comprising the universal nucleotide. Cleavage of the scaffold polynucleotide thereby releases the polynucleotide ligation molecule from the scaffold polynucleotide but leads to the retention of the first nucleotide of that cycle attached to the first strand of the cleaved scaffold polynucleotide. Cleavage of the scaffold polynucleotide leaves in place a cleaved double-stranded scaffold polynucleotide comprising a singe-base overhang at the cleaved end. The first nucleotide of the predefined sequence occupies a position (n) as the terminal nucleotide of the first strand of the cleaved double-stranded scaffold polynucleotide and overhangs the terminal nucleotide of the second strand.

The second strand of the ligated scaffold polynucleotide is already provided with a single-strand break or “nick” in this exemplary method, thus only cleavage of the first strand is necessary to provide a double-strand break in the scaffold polynucleotide. Furthermore, as noted previously, in this exemplary method version cleavage generates a cleaved double-stranded scaffold polynucleotide with a single-base overhang, with the terminal nucleotide of the first strand overhanging the terminal nucleotide of the second strand, and the universal nucleotide occupies position n+2 in the first strand prior to the cleavage step. To obtain such a cleaved double-stranded scaffold polynucleotide with a singe-base overhang when the universal nucleotide occupies position n+2 in the first strand, the ligated first strand is cleaved at a specific position relative to the universal nucleotide. When the first strand of the scaffold polynucleotide is cleaved between nucleotide positions n+1 and n the polynucleotide ligation molecule is released from the scaffold polynucleotide (see the structure depicted as exiting the synthesis cycle immediately after cleavage step 3 (603) in FIG. 6) except that the first nucleotide of that cycle derived from the first polynucleotide ligation molecule in step (2) is retained in the scaffold polynucleotide attached to the first strand of the cleaved scaffold polynucleotide.

A hydroxyl group, or any other suitable 3′ ligatable group, should continue to be attached to the terminal nucleotide of the second strand of the cleaved scaffold polynucleotide at the cleavage site. This ensures that the second strand of the cleaved scaffold polynucleotide can be ligated to the synthesis strand of the second polynucleotide ligation molecule in the second extension/ligation step (4). Cleavage is performed so that the terminal nucleotide of the first strand of the cleaved scaffold polynucleotide retains ligatable group, typically a phosphate group, or any other suitable 5′ ligatable group, at the 5′ end of the first strand.

Thus in method version 6 the universal nucleotide occupies position n+2 in the synthesis/first strand at step (2) and the first strand is cleaved between nucleotide positions n+1 and n at step (3).

Preferably, the first strand is cleaved by cleavage of the phosphodiester bond between nucleotide positions n+1 and n (the first phosphodiester bond of the first strand relative to the position of the universal nucleotide, in the direction distal to the ligated polynucleotide ligation molecule/proximal to the first strand).

The first strand may be cleaved by cleavage of one ester bond of the phosphodiester bond between nucleotide positions n+1 and n.

Preferably the first strand is cleaved by cleavage of the first ester bond relative to nucleotide position n+1.

Any suitable mechanism may be employed to effect cleavage of the first strand between nucleotide positions n+1 and n when the universal nucleotide occupies position n+2.

Cleavage of the first strand between nucleotide positions n+1 and n as described above may be performed by the action of an enzyme.

Cleavage of the first strand between nucleotide positions n+1 and n when the universal nucleotide occupies position n+2 in the first strand, as described above, may be performed by the action of an enzyme such as Endonuclease V.

One mechanism of cleaving the first strand between nucleotide positions n+1 and n at a cleavage site defined by a sequence comprising a universal nucleotide which is occupying position n+2 in the first strand is described in analogous fashion in Example 3.

The mechanism described is exemplary and other mechanisms could be employed, provided that the cleavage arrangement described above is achieved.

In this exemplary mechanism an endonuclease enzyme is employed. In the exemplified method the enzyme is Endonuclease V. Other enzymes, molecules or chemicals could be used provided that the first strand is cleaved between nucleotide positions n+1 and n when the universal nucleotide occupies position n+2 in the first strand.

### Step 4—Ligation of a Second Polynucleotide Ligation Molecule to the Scaffold Polynucleotide and Incorporation of One or More Further Nucleotides of the Predefined Sequence

In step (4) of the method a second double-stranded polynucleotide ligation molecule is ligated (604) to the cleaved scaffold polynucleotide in a sticky-(complementary)-ended ligation reaction by the action of an enzyme having ligase activity.

The second polynucleotide ligation molecule comprises a synthesis strand and a helper strand hybridised thereto. The second polynucleotide ligation molecule further comprises a complementary ligation end comprising in the synthesis strand a universal nucleotide and a further nucleotide of the predefined sequence.

The complementary ligation end of the second polynucleotide ligation molecule is structured such that the terminal nucleotide of the synthesis strand is the first nucleotide of the predefined sequence to be incorporated into the second strand of the cleaved scaffold polynucleotide in any given cycle of synthesis.

The complementary ligation end comprises a single-base overhang. The terminal nucleotide of the synthesis strand is unpaired and overhangs the terminal nucleotide of the helper strand. In FIG. 6 the terminal nucleotide of the synthesis strand is depicted as “A” for illustrative purposes only. It can be any nucleotide, nucleotide analogue or nucleotide derivative.

The universal nucleotide is the penultimate nucleotide at the terminal end of the synthesis strand at the complementary ligation end of the second polynucleotide ligation molecule. The universal nucleotide forms a nucleotide pair with the terminal nucleotide of the helper strand at the complementary ligation end of the second polynucleotide ligation molecule. In FIG. 6 the terminal nucleotide of the helper strand at the complementary ligation end is depicted as “X” for illustrative purposes only. It can be any nucleotide, nucleotide analogue or nucleotide derivative.

The terminal nucleotide of the synthesis strand at the complementary ligation end of the second polynucleotide ligation molecule is depicted in FIG. 6 at the 5′ end of the synthesis strand. This nucleotide is a ligatable nucleotide and is provided with a phosphate group or any other suitable 5′ ligatable group. The terminal nucleotide of the helper strand at the complementary ligation end of the second polynucleotide ligation molecule is depicted in FIG. 6 at the 3′ end of the helper strand. This nucleotide is provided as a non-ligatable nucleotide and comprises a non-ligatable 2′,3′-dideoxynucleotide or a 2′-deoxynucleotide, or any other suitable 3′ non-ligatable nucleotide.

The terminal nucleotide of the synthesis strand, i.e. the first nucleotide of the predefined sequence of that cycle to be incorporated into the second strand of the cleaved scaffold polynucleotide, occupies nucleotide position n in the synthesis strand. With reference to the definition of position n in the first extension/ligation reaction, position n refers to the nucleotide position in the synthesis strand of the second polynucleotide ligation molecule which is occupied by a nucleotide which will be a partner nucleotide for the first nucleotide of step (2) in the first strand following attachment of the partner nucleotide to the terminal end of the second strand after the second extension/ligation reaction. Position n also refers to the nucleotide position which is occupied by the said partner nucleotide following its attachment to the terminal end of the second strand after the second extension/ligation reaction. Position n also refers to the nucleotide position in the first strand of the scaffold polynucleotide which is occupied by the first nucleotide following its attachment to the terminal end of the first strand after ligation in step (2).

The universal nucleotide in the synthesis strand of the second polynucleotide ligation molecule occupies position n+1.

The complementary ligation end of the second polynucleotide ligation molecule is configured so that it will compatibly join with the overhanging end of the cleaved scaffold polynucleotide, generated in step (3), when subjected to suitable ligation conditions. Upon ligation of the synthesis strand of the second polynucleotide ligation molecule and the second strand of the scaffold polynucleotide, the terminal nucleotide of the synthesis strand of the second polynucleotide ligation becomes incorporated into the second strand of the cleaved scaffold polynucleotide. Because the terminal nucleotide of the helper strand of the second polynucleotide ligation molecule is a non-ligatable nucleotide, the ligase enzyme will be prevented from ligating the helper strand of the second polynucleotide ligation molecule and the first strand of the cleaved scaffold polynucleotide, thus creating a single-strand break or “nick” between the helper strand of the second polynucleotide ligation molecule and the first strand of the cleaved scaffold polynucleotide.

Ligation of the second polynucleotide ligation molecule to the cleaved scaffold polynucleotide extends the length of the second strand of the double-stranded scaffold polynucleotide of step (3) and wherein the terminal nucleotide of the synthesis strand of the second polynucleotide ligation molecule sequence is incorporated into the second strand of the scaffold polynucleotide.

Ligation may be performed by any suitable means. Ligation may typically and preferably be performed by an enzyme having ligase activity. For example, ligation may be performed with T3 DNA ligase or T4 DNA ligase or functional variants or equivalents thereof or other enzymes described herein. The use of such enzymes will result in the maintenance of the single-strand break, since the terminal nucleotide of the helper strand is provided such that it cannot act as a substrate for ligase, as described above.

Upon ligation, the terminal nucleotide of the synthesis strand of the second polynucleotide ligation molecule pairs with the terminal nucleotide of the first strand of the cleaved scaffold polynucleotide generated in step (3), thus forming a nucleotide pair.

### Step 5—Second Cleavage Step

In step (5) of the method the ligated scaffold polynucleotide is cleaved (605) at a cleavage site. The cleavage site is defined by a sequence comprising the universal nucleotide in the synthesis strand of the ligated second polynucleotide ligation molecule. Cleavage results in a double-strand break in the scaffold polynucleotide. Cleavage of the scaffold polynucleotide (step 5) results in loss of the helper strand of the ligated second polynucleotide ligation molecule and loss of the synthesis strand comprising the universal nucleotide. Cleavage of the scaffold polynucleotide thereby releases the second polynucleotide ligation molecule from the scaffold polynucleotide but leads to the retention of the second nucleotide of that cycle attached to the second strand of the cleaved scaffold polynucleotide. Cleavage of the scaffold polynucleotide leaves in place a cleaved double-stranded scaffold polynucleotide comprising a blunt end. The second nucleotide of the predefined sequence incorporated in step (4) occupies a position (n) as the terminal nucleotide of the second strand of the cleaved double-stranded scaffold polynucleotide and is paired with the first/terminal nucleotide of the first strand of the cleaved double-stranded scaffold polynucleotide which was incorporated in step (2) and which occupies position n in the first strand. In FIG. 6 these nucleotides are depicted (605) as “A” and “T” for illustrative purposes only. Each of these nucleotides can be any nucleotide, nucleotide analogue or nucleotide derivative and the pair may or may not be complementary. Preferably they are complementary.

Prior to the second cleavage step the first strand of the ligated scaffold polynucleotide is already provided with a single-strand break or “nick” in this exemplary method, thus only cleavage of the second strand is necessary to provide a double-strand break in the scaffold polynucleotide. Furthermore, as noted previously in this exemplary method version cleavage generates a cleaved double-stranded scaffold polynucleotide with a blunt end, and the universal nucleotide occupies position n+1 in the second strand prior to the second cleavage step. To obtain such a cleaved double-stranded scaffold polynucleotide with a blunt end when the universal nucleotide occupies position n+1 in the second strand, the second strand is cleaved at a specific position relative to the universal nucleotide. When the second strand of the scaffold polynucleotide is cleaved between nucleotide positions n+1 and n the polynucleotide ligation molecule is released from the scaffold polynucleotide (see the structure depicted as exiting the synthesis cycle immediately after cleavage step 5 (605) in FIG. 6) except that the first nucleotide of that cycle derived from the second polynucleotide ligation molecule in step (4) is retained in the scaffold polynucleotide attached to the second strand of the cleaved scaffold polynucleotide.

A phosphate group, or any other suitable 5′ ligatable group, should continue to be attached to the terminal nucleotide of the first strand of the cleaved scaffold polynucleotide at the cleavage site. This ensures that the first strand of the cleaved scaffold polynucleotide can be ligated to the synthesis strand of the first polynucleotide ligation molecule in the first extension/ligation step (2) of the next cycle of synthesis. Cleavage is performed so that the terminal nucleotide of the second strand of the cleaved scaffold polynucleotide retains ligatable group, typically a hydroxyl group or any other suitable 3′ ligatable group, at the 3′ end of the first strand.

Thus in method version 6 the universal nucleotide occupies position n+1 in the synthesis/second strand at step (4) and the second strand is cleaved between nucleotide positions n+1 and n at step (5).

Preferably, the second strand is cleaved by cleavage of the phosphodiester bond between nucleotide positions n+1 and n (the first phosphodiester bond of the second strand relative to the position of the universal nucleotide, in the direction distal to the ligated polynucleotide ligation molecule/proximal to the second strand).

The second strand may be cleaved by cleavage of one ester bond of the phosphodiester bond between nucleotide positions n+1 and n.

Preferably the second strand is cleaved by cleavage of the first ester bond relative to nucleotide position n+1.

Any suitable mechanism may be employed to effect cleavage of the second strand between nucleotide positions n+1 and n when the universal nucleotide occupies position n+1.

Cleavage of the second strand between nucleotide positions n+1 and n as described above may be performed by the action of an enzyme.

Cleavage of the second strand between nucleotide positions n+1 and n as described above may be performed as a two-step cleavage process.

The first cleavage step of a two-step cleavage process may comprise removing the universal nucleotide from the second strand thus forming an abasic site at position n+1, and the second cleavage step may comprise cleaving the second strand at the abasic site, between positions n+1 and n.

One mechanism of cleaving the second strand at a cleavage site defined by a sequence comprising a universal nucleotide in the manner outlined above is described in analogous fashion in Example 2. The cleavage mechanism described in Example 2 is exemplary and other mechanisms could be employed, provided that the cleaved double-stranded scaffold polynucleotide described above is achieved.

In the first cleavage step of a two-step cleavage process the universal nucleotide is removed from the second strand whilst leaving the sugar-phosphate backbone intact. This can be achieved by the action of an enzyme which can specifically excise a single universal nucleotide from a double-stranded polynucleotide. In the exemplified cleavage methods the universal nucleotide is inosine and inosine is excised from the strand by the action of an enzyme, thus forming an abasic site. In the exemplified cleavage method the enzyme is a 3-methyladenine DNA glycosylase enzyme, specifically human alkyladenine DNA glycosylase (hAAG). Other enzymes, molecules or chemicals could be used provided that an abasic site is formed. The nucleotide-excising enzyme may be an enzyme which catalyses the release of uracil from polynucleotides, such as Uracil-DNA Glycosylase (UDG).

In the second step of a two-step cleavage process the second strand is cleaved at the abasic site by making a single-strand break. In the exemplified methods the strand is cleaved by the action of a chemical which is a base, such as NaOH. Alternatively, an organic chemical such as N,N′-dimethylethylenediamine may be used. Alternatively, enzymes having abasic site lyase activity, such as AP Endonuclease 1, Endonuclease III (Nth), or Endonuclease VIII, may be used. These enzymes cleave the DNA backbone at a phosphate group which is positioned 5′ relative to the abasic site. Cleavage thus exposes an OH group at the 3′ terminal end of the second strand which provides a terminal 3′ nucleotide which is ligatable in the second ligation step in the next cycle. Other enzymes, molecules or chemicals could be used provided that the second strand is cleaved at the abasic site as described.

Thus in embodiments wherein the universal nucleotide is at position n+1 of the second strand at step (4) and the second strand is cleaved between positions n+1 and n, a first cleavage step may be performed with a nucleotide-excising enzyme. An example of such an enzyme is a 3-methyladenine DNA glycosylase enzyme, such as human alkyladenine DNA glycosylase (hAAG). The second cleavage step may be performed with a chemical which is a base, such as NaOH. The second step may be performed with an organic chemical having abasic site cleavage activity such as N,N′-dimethylethylenediamine. The second step may be performed with an enzyme having abasic site lyase activity such as Endonuclease VIII or Endonuclease III.

Cleavage of the second strand between nucleotide positions n+1 and n as described above may also be performed as a one-step cleavage process. Examples of enzymes which may be used in any such process include Endonuclease III, Endonuclease VIII. Other enzymes which may be used in any such process include enzymes which cleave 8-oxoguanosine, such as formamidopyrimidine DNA glycosylase (Fpg) and 8-oxoguanine DNA glycosylase (hOGG1), which cleaves the DNA backbone so as to leave a phosphate group at the 3′ terminal end of the cleaved second strand, which can then be removed by Endonuclease IV or T4 polynucleotide kinase to expose an OH group which is ligatable in the second ligation step in the next cycle.

In synthesis method version 6 it will be noted that in any given cycle of synthesis, after the second cleavage step (step 5) the nucleotide positions which are occupied by the terminal nucleotide of the first and second strands at the cleaved end are both defined as nucleotide position n. These nucleotide positions are defined as nucleotide position n−1 in the next cycle of synthesis.

### Further Cycles

Following completion of the first cycle of synthesis, second and further cycles of synthesis may be performed using the same method steps.

The cleavage product of step (5) of the previous cycle is provided (in step 6) as the double-stranded scaffold polynucleotide for the next cycle of synthesis.

In step (7) of the next and each further cycle of synthesis a further first double-stranded polynucleotide ligation molecule is ligated to the cleavage product of step (5) of the previous cycle. The polynucleotide ligation molecule may be structured in the same way as described above for step (2) of the previous cycle, except that the further first polynucleotide ligation molecule comprises further first nd nucleotide of the further cycle of synthesis to be incorporated into the first strand. In step (7) the further first polynucleotide ligation molecule may be ligated to the cleavage product of step (5) of the previous cycle in the same way as described above for step (2).

In step (8) of the next and each further cycle of synthesis the ligated scaffold polynucleotide is subjected to a further first cleavage step at the cleavage site. Cleavage results in a double-strand break in the scaffold polynucleotide. Cleavage of the scaffold polynucleotide (step 8) results in loss of the helper strand and loss of the support strand comprising the universal nucleotide in the further first polynucleotide ligation molecule. Cleavage of the scaffold polynucleotide thereby releases the further first polynucleotide ligation molecule from the scaffold polynucleotide but leads to the retention of the further first nucleotide of that further cycle derived from the further first polynucleotide ligation molecule attached to the synthesis strand of the cleaved scaffold polynucleotide. Cleavage of the scaffold polynucleotide leaves in place a cleaved double-stranded scaffold polynucleotide comprising the further first nucleotide of the further cycle at the terminal end of the synthesis strand of the scaffold polynucleotide. Cleavage results in a single-base overhang with the terminal nucleotide of the first strand overhanging the terminal nucleotide of the second strand. Cleavage at step (8) may be performed in the same way as described above for step (4).

In step (9) of the next and each further cycle of synthesis a further second double-stranded polynucleotide ligation molecule is ligated to the cleavage product of step (8). The further second polynucleotide ligation molecule may be structured in the same way as described above for step (8) of the previous cycle, except that the further second polynucleotide ligation molecule comprises further first nucleotide of the further cycle of synthesis to be incorporated into the second strand. In step (9) the further second polynucleotide ligation molecule may be ligated to the cleavage product of step (8) in the same way as described above for step (4).

In step (10) of the next and each further cycle of synthesis the ligated scaffold polynucleotide is subjected to a further second cleavage step at the cleavage site. Cleavage results in a double-strand break in the scaffold polynucleotide. Cleavage of the scaffold polynucleotide (step 10) results in loss of the helper strand and loss of the support strand comprising the universal nucleotide in the further second polynucleotide ligation molecule. Cleavage of the scaffold polynucleotide thereby releases the further second polynucleotide ligation molecule from the scaffold polynucleotide but leads to the retention of the further first nucleotide of that further cycle derived from the further second polynucleotide ligation molecule attached to the synthesis strand of the cleaved scaffold polynucleotide. Cleavage of the scaffold polynucleotide leaves in place a cleaved blunt-ended double-stranded scaffold polynucleotide comprising the further first nucleotide of the further cycle derived from the further second polynucleotide ligation molecule at the terminal end of the second strand of the scaffold polynucleotide. Cleavage at step (10) may be performed in the same way as described above for step (5).

### Variants of Synthesis Methods

A number of different variants of the above-described synthesis methods are envisaged within the scope of the invention and are described in more detail below.

### Variants of Synthesis Method Version 1

Variants of synthesis method of the invention version 1 are provided wherein the method is performed in the same way as synthesis method version 1 described above except for the variations described below.

Synthesis method of the invention version 1 and variants thereof can be defined generically via formulae.

In the first ligation step (step 2) the complementary ligation end of the first polynucleotide ligation molecule is structured such that the universal nucleotide occupies a nucleotide position in the synthesis strand defined by the formula n+x and is paired with a partner nucleotide in the helper strand which is x−1 positions removed from the terminal nucleotide of the helper strand at the complementary ligation end. For example, if x=2 the universal nucleotide occupies nucleotide position n+2 in the synthesis strand. Position n+2 is the second nucleotide position in the synthesis strand relative to nucleotide position n in the direction distal to the complementary ligation end. The nucleotide at position n in the synthesis strand is the first nucleotide of the predefined sequence to be incorporated into the first strand of the scaffold polynucleotide during the first ligation step, and pairs with the terminal nucleotide of the second strand. If the universal nucleotide occupies position n+2 it is paired with a partner nucleotide in the helper strand which is 2-1 positions, i.e. 1 positon, removed from the terminal nucleotide of the helper strand at the complementary ligation end. In other words, the universal nucleotide occupying position n+2 would be paired with the penultimate nucleotide of the helper strand at the complementary ligation end.

In the first cleavage step (step 3) the synthesis strand of the scaffold polynucleotide is always cleaved between positions n+2 and n+1, regardless of the number which is selected for x in order to define the position in the synthesis strand of the universal nucleotide in the first polynucleotide ligation molecule.

In these methods the value selected for x in steps (2) and (3) is a whole number from 2 to 10 or more.

Thus in these particular variants of synthesis method version 1 the position of the first cleavage site is held constant between positions n+2 and n+1, and the position of the universal nucleotide relative to the cleavage site is increased by moving the position of the universal nucleotide in the direction distal to the cleavage site by a number of nucleotide positions determined by the number selected for x.

The structuring of the second polynucleotide ligation molecule and the second cleavage step can be varied, either independently or in conjunction with the variations made to the first polynucleotide ligation molecule and the first cleavage step.

Thus in the second ligation step (step 4) the complementary ligation end of the second polynucleotide ligation molecule may be structured such that the universal nucleotide occupies a nucleotide position in the synthesis strand defined by the formula n+x and is paired with a partner nucleotide in the helper strand which is n+x−2 positions removed from the terminal nucleotide of the helper strand at the complementary ligation end. For example, if x=3 the universal nucleotide occupies nucleotide position n+3 in the synthesis strand. Position n+3 is the third nucleotide position in the synthesis strand relative to nucleotide position n in the direction distal to the complementary ligation end. If the universal nucleotide occupies position n+3 it is paired with a partner nucleotide in the helper strand which is n+3−2 positions, i.e. 1 positon, removed from the terminal nucleotide of the helper strand at the complementary ligation end. In other words, the universal nucleotide occupying position n+3 would be paired with the penultimate nucleotide of the helper strand at the complementary ligation end.

In the second cleavage step (step 5) the synthesis strand of the scaffold polynucleotide is always cleaved between positions n+3 and n+2, regardless of the number which is selected for y in order to define the position in the synthesis strand of the universal nucleotide in the second polynucleotide ligation molecule.

In these methods the value selected for x in steps (4) and (5) is a whole number from 3 to 10 or more.

Thus in these particular variants of synthesis method version 1 the position of the second cleavage site is held constant between positions n+3 and n+2, and the position of the universal nucleotide relative to the cleavage site is increased by moving the position of the universal nucleotide in the direction distal to the cleavage site by a number of nucleotide positions determined by the number selected for x.

The number selected for x in the first ligation and cleavage steps can be varied independently of the number selected for x in the second ligation and cleavage steps. For example, in these particular variant synthesis methods of the invention the number selected for x in the second ligation and cleavage steps could be held constant and the number selected for x in the first ligation and cleavage steps could be varied. Alternatively, the number selected for x in the first ligation and cleavage steps could be held constant and the number selected for x in the second ligation and cleavage steps could be varied. Yet further, combination methods are envisaged wherein any values for x could be selected for the first ligation and cleavage steps and in combination any values for x could be selected for the second ligation and cleavage steps.

Thus the invention provides for a variant method according to method of the invention version 1, wherein in step (2) the universal nucleotide is positioned in the synthesis strand of the first polynucleotide ligation molecule at a position defined by the formula n+x and wherein in step (3) the ligated first strand of the scaffold polynucleotide is cleaved between positions n+2 and n+1, wherein x is a number of nucleotide positions relative to position n in the direction distal to the complementary ligation end and wherein x is a whole number from 2 to 10 or more.

Independently, the invention provides for a variant method according to method of the invention version 1, wherein in step (4) the universal nucleotide is positioned in the synthesis strand of the second polynucleotide ligation molecule at a position defined by the formula n+x and wherein in step (5) the ligated second strand of the scaffold polynucleotide is cleaved between positions n+3 and n+2, wherein x is a number of nucleotide positions relative to position n in the direction distal to the complementary ligation end and wherein x is a whole number from 3 to 10 or more.

In combination, the invention provides for a variant method according to synthesis method of the invention version 1, wherein in step (2) the universal nucleotide is positioned in the synthesis strand of the first polynucleotide ligation molecule at a position defined by the formula n+x, wherein in step (3) the ligated first strand of the scaffold polynucleotide is cleaved between positions n+2 and n+1, wherein x is a whole number from 2 to 10 or more; and wherein in step (4) the universal nucleotide is positioned in the synthesis strand of the second polynucleotide ligation molecule at a position defined by the formula n+x and wherein in step (5) the ligated second strand of the scaffold polynucleotide is cleaved between positions n+3 and n+2 wherein x is a whole number from 3 to 10 or more; and wherein in steps (2) and (4) x is a number of nucleotide positions relative to position n in the direction distal to the complementary ligation end.

According to this combination scheme the selection of 2 for the value of x for the first ligation and cleavage steps and the selection of 3 for the value of x for the second ligation and cleavage steps will give rise to a method which is defined as described for synthesis method of the invention version 1. The selection of 2 for the value of x for the first ligation and cleavage steps and the selection of 4 for the value of x for the second ligation and cleavage steps will give rise to a method which is defined as described for synthesis method of the invention version 2. Thus synthesis method of the invention 2 can be defined as a variant of synthesis method of the invention 1 when considered in the context of the formulae set out above.

### Variants of Synthesis Method Version 3

Variants of synthesis method of the invention version 3 are provided wherein the method is performed in the same way as synthesis method version 3 described above except for the variations described below.

Synthesis method of the invention version 3 and variants thereof can be defined generically via formulae.

In the first ligation step (step 2) the complementary ligation end of the first polynucleotide ligation molecule is structured such that the universal nucleotide occupies a nucleotide position in the synthesis strand defined by the formula n+x and is paired with a partner nucleotide in the helper strand which is x positions removed from the terminal nucleotide of the helper strand at the complementary ligation end. For example, if x=1 the universal nucleotide occupies nucleotide position n+1 in the synthesis strand. Position n+1 is the second nucleotide position in the synthesis strand relative to nucleotide position n in the direction distal to the complementary ligation end. The nucleotide at position n in the synthesis strand is the first nucleotide of the predefined sequence to be incorporated into the first strand of the scaffold polynucleotide during the first ligation step, and pairs with the first nucleotide of the predefined sequence to be incorporated into the second strand of the scaffold polynucleotide during the second ligation step. If the universal nucleotide occupies position n+1 it is paired with a partner nucleotide in the helper strand which is x positions, i.e. 1 positon, removed from the terminal nucleotide of the helper strand at the complementary ligation end. In other words, the universal nucleotide occupying position n+1 would be paired with the penultimate nucleotide of the helper strand at the complementary ligation end.

In the first cleavage step (step 3) the synthesis strand of the scaffold polynucleotide is always cleaved between positions n+1 and n, regardless of the number which is selected for x in order to define the position in the synthesis strand of the universal nucleotide in the first polynucleotide ligation molecule.

Thus in these particular variants of synthesis method version 3 the position of the first cleavage site is held constant between positions n+1 and n, and the position of the universal nucleotide relative to the cleavage site is increased by moving the position of the universal nucleotide in the direction distal to the cleavage site by a number of nucleotide positions determined by the number selected for x.

The structuring of the second polynucleotide ligation molecule and the second cleavage step can be varied, either independently or in conjunction with the variations made to the first polynucleotide ligation molecule and the first cleavage step.

Thus in the second ligation step (step 4) the complementary ligation end of the second polynucleotide ligation molecule may be structured such that the universal nucleotide occupies a nucleotide position in the synthesis strand defined by the formula n+x and is paired with a partner nucleotide in the helper strand which is x−1 positions removed from the terminal nucleotide of the helper strand at the complementary ligation end. For example, if x=2 the universal nucleotide occupies nucleotide position n+2 in the synthesis strand. Position n+2 is the second nucleotide position in the synthesis strand relative to nucleotide position n in the direction distal to the complementary ligation end. If the universal nucleotide occupies position n+2 it is paired with a partner nucleotide in the helper strand which is 2−1 positions, i.e. 1 positon, removed from the terminal nucleotide of the helper strand at the complementary ligation end. In other words, the universal nucleotide occupying position n+2 would be paired with the penultimate nucleotide of the helper strand at the complementary ligation end.

In the second cleavage step (step 5) the synthesis strand of the scaffold polynucleotide is always cleaved between positions n+1 and n, regardless of the number which is selected for x in order to define the position in the synthesis strand of the universal nucleotide in the second polynucleotide ligation molecule.

Thus in these particular variants of synthesis method version 3 the position of the second cleavage site is held constant between positions n+1 and n, and the position of the universal nucleotide relative to the cleavage site is increased by moving the position of the universal nucleotide in the direction distal to the cleavage site by a number of nucleotide positions determined by the number selected for x.

The number selected for x in the first ligation and cleavage steps can be varied independently of the number selected for x in the second ligation and cleavage steps. For example, in these particular variant synthesis methods of the invention the number selected for x in the second ligation and cleavage steps could be held constant and the number selected for x in the first ligation and cleavage steps could be varied. Alternatively, the number selected for x in the first ligation and cleavage steps could be held constant and the number selected for x in the second ligation and cleavage steps could be varied. Yet further, combination methods are envisaged wherein any values for x could be selected for the first ligation and cleavage steps and in combination any values for x could be selected for the second ligation and cleavage steps.

Thus the invention provides for a variant method according to method of the invention version 3, wherein in step (2) the universal nucleotide is positioned in the synthesis strand of the first polynucleotide ligation molecule at a position defined by the formula n+x and wherein in step (3) the ligated first strand of the scaffold polynucleotide is cleaved between positions n+1 and n, wherein x is a number of nucleotide positions relative to position n in the direction distal to the complementary ligation end and wherein x is a whole number from 1 to 10 or more.

Independently, the invention provides for a variant method according to method of the invention version 3, wherein in step (4) the universal nucleotide is positioned in the synthesis strand of the second polynucleotide ligation molecule at a position defined by the formula n+x and wherein in step (5) the ligated second strand of the scaffold polynucleotide is cleaved between positions n+1 and n, wherein x is a number of nucleotide positions relative to position n in the direction distal to the complementary ligation end and wherein x is a whole number from 1 to 10 or more.

In combination, the invention provides for a variant method according to synthesis method of the invention version 3, wherein in step (2) the universal nucleotide is positioned in the synthesis strand of the first polynucleotide ligation molecule at a position defined by the formula n+x, wherein in step (3) the ligated first strand of the scaffold polynucleotide is cleaved between positions n+1 and n, wherein x is a whole number from 1 to 10 or more; and wherein in step (4) the universal nucleotide is positioned in the synthesis strand of the second polynucleotide ligation molecule at a position defined by the formula n+x and wherein in step (5) the ligated second strand of the scaffold polynucleotide is cleaved between positions n+1 and n wherein x is a whole number from 1 to 10 or more; and wherein in steps (2) and (4) x is a number of nucleotide positions relative to position n in the direction distal to the complementary ligation end.

According to this combination scheme the selection of 1 for the value of x for the first ligation and cleavage steps and the selection of 1 for the value of x for the second ligation and cleavage steps will give rise to a method which is defined as described for synthesis method of the invention version 3. The selection of 1 for the value of x for the first ligation and cleavage steps and the selection of 2 for the value of x for the second ligation and cleavage steps will give rise to a method which is defined as described for synthesis method of the invention version 4. Thus synthesis method of the invention version 4 can be defined as a variant of synthesis method of the invention version 3 when considered in the context of the formulae set out above.

### Variants of Synthesis Method Version 5

Variants of synthesis method of the invention version 5 are provided wherein the method is performed in the same way as synthesis method version 5 described above except for the variations described below.

Synthesis method of the invention version 5 and variants thereof can be defined generically via formulae.

In the first ligation step (step 2) the complementary ligation end of the first polynucleotide ligation molecule is structured such that the universal nucleotide occupies a nucleotide position in the synthesis strand defined by the formula n+x and is paired with a partner nucleotide in the helper strand which is x positions removed from the terminal nucleotide of the helper strand at the complementary ligation end. For example, if x=1 the universal nucleotide occupies nucleotide position n+1 in the synthesis strand. Position n+1 is the second nucleotide position in the synthesis strand relative to nucleotide position n in the direction distal to the complementary ligation end. The nucleotide at position n in the synthesis strand is the first nucleotide of the predefined sequence to be incorporated into the first strand of the scaffold polynucleotide during the first ligation step, and pairs with the first nucleotide of the predefined sequence to be incorporated into the second strand of the scaffold polynucleotide during the second ligation step. If the universal nucleotide occupies position n+1 it is paired with a partner nucleotide in the helper strand which is x positions, i.e. 1 positon, removed from the terminal nucleotide of the helper strand at the complementary ligation end. In other words, the universal nucleotide occupying position n+1 would be paired with the penultimate nucleotide of the helper strand at the complementary ligation end.

In the first cleavage step (step 3) the synthesis strand of the scaffold polynucleotide is always cleaved between positions n+1 and n, regardless of the number which is selected for x in order to define the position in the synthesis strand of the universal nucleotide in the first polynucleotide ligation molecule.

Thus in these particular variants of synthesis method version 5 the position of the first cleavage site is held constant between positions n+1 and n, and the position of the universal nucleotide relative to the cleavage site is increased by moving the position of the universal nucleotide in the direction distal to the cleavage site by a number of nucleotide positions determined by the number selected for x.

The structuring of the second polynucleotide ligation molecule and the second cleavage step can be varied, either independently or in conjunction with the variations made to the first polynucleotide ligation molecule and the first cleavage step.

Thus in the second ligation step (step 4) the complementary ligation end of the second polynucleotide ligation molecule may be structured such that the universal nucleotide occupies a nucleotide position in the synthesis strand defined by the formula n+x and is paired with a partner nucleotide in the helper strand which is x−1 positions removed from the terminal nucleotide of the helper strand at the complementary ligation end. For example, if x=2 the universal nucleotide occupies nucleotide position n+2 in the synthesis strand. Position n+2 is the second nucleotide position in the synthesis strand relative to nucleotide position n in the direction distal to the complementary ligation end. If the universal nucleotide occupies position n+2 it is paired with a partner nucleotide in the helper strand which is 2-1 positions, i.e. 1 positon, removed from the terminal nucleotide of the helper strand at the complementary ligation end. In other words, the universal nucleotide occupying position n+2 would be paired with the penultimate nucleotide of the helper strand at the complementary ligation end.

In the second cleavage step (step 5) the synthesis strand of the scaffold polynucleotide is always cleaved between positions n+1 and n, regardless of the number which is selected for x in order to define the position in the synthesis strand of the universal nucleotide in the second polynucleotide ligation molecule.

Thus in these particular variants of synthesis method version 5 the position of the second cleavage site is held constant between positions n+1 and n, and the position of the universal nucleotide relative to the cleavage site is increased by moving the position of the universal nucleotide in the direction distal to the cleavage site by a number of nucleotide positions determined by the number selected for x.

The number selected for x in the first ligation and cleavage steps can be varied independently of the number selected for x in the second ligation and cleavage steps. For example, in these particular variant synthesis methods of the invention the number selected for x in the second ligation and cleavage steps could be held constant and the number selected for x in the first ligation and cleavage steps could be varied. Alternatively, the number selected for x in the first ligation and cleavage steps could be held constant and the number selected for x in the second ligation and cleavage steps could be varied. Yet further, combination methods are envisaged wherein any values for x could be selected for the first ligation and cleavage steps and in combination any values for x could be selected for the second ligation and cleavage steps.

Thus the invention provides for a variant method according to method of the invention version 5, wherein in step (2) the universal nucleotide is positioned in the synthesis strand of the first polynucleotide ligation molecule at a position defined by the formula n+x and wherein in step (3) the ligated first strand of the scaffold polynucleotide is cleaved between positions n+1 and n, wherein x is a number of nucleotide positions relative to position n in the direction distal to the complementary ligation end and wherein x is a whole number from 1 to 10 or more.

Independently, the invention provides for a variant method according to method of the invention version 5, wherein in step (4) the universal nucleotide is positioned in the synthesis strand of the second polynucleotide ligation molecule at a position defined by the formula n+x and wherein in step (5) the ligated second strand of the scaffold polynucleotide is cleaved between positions n+1 and n, wherein x is a number of nucleotide positions relative to position n in the direction distal to the complementary ligation end and wherein x is a whole number from 1 to 10 or more.

In combination, the invention provides for a variant method according to synthesis method of the invention version 5, wherein in step (2) the universal nucleotide is positioned in the synthesis strand of the first polynucleotide ligation molecule at a position defined by the formula n+x, wherein in step (3) the ligated first strand of the scaffold polynucleotide is cleaved between positions n+1 and n, wherein x is a whole number from 1 to 10 or more; and wherein in step (4) the universal nucleotide is positioned in the synthesis strand of the second polynucleotide ligation molecule at a position defined by the formula n+x and wherein in step (5) the ligated second strand of the scaffold polynucleotide is cleaved between positions n+1 and n wherein x is a whole number from 1 to 10 or more; and wherein in steps (2) and (4) x is a number of nucleotide positions relative to position n in the direction distal to the complementary ligation end.

According to this combination scheme the selection of 1 for the value of x for the first ligation and cleavage steps and the selection of 1 for the value of x for the second ligation and cleavage steps will give rise to a method which is defined as described for synthesis method of the invention version 5. The selection of 1 for the value of x for the first ligation and cleavage steps and the selection of 2 for the value of x for the second ligation and cleavage steps will give rise to a method which is defined as described for synthesis method of the invention version 6. Thus synthesis method of the invention version 6 can be defined as a variant of synthesis method of the invention version 5 when considered in the context of the formulae set out above.

### Further Variants of Synthesis Method Version 3

Further variants of synthesis method of the invention version 3 are provided wherein the method is performed in the same way as synthesis method version 3 described above except for the variations described below. In these variant methods two or more nucleotides can be incorporated into the first strand during the first ligation reaction, and subsequently two or more corresponding nucleotides can be incorporated into the second strand during the second ligation reaction. These further variant methods can also be defined generically via formulae. Generic variant methods and two specific illustrative variant methods, synthesis method versions 7 and 8, are described below.

Synthesis method of the invention version 7 is an illustrative specific example of a further variant of synthesis method version 3 and which can be defined generically as follows.

In the first ligation step (step 2) the complementary ligation end of the first polynucleotide ligation molecule is structured such that the universal nucleotide occupies a nucleotide position in the synthesis strand defined by the formula n+1+x and is paired with a partner nucleotide at the same position in the helper strand at the complementary ligation end. For example, if x=1 the universal nucleotide occupies nucleotide position n+2 in the synthesis strand. Position n+2 is the second nucleotide position in the synthesis strand relative to nucleotide position n in the direction distal to the complementary ligation end. The nucleotide at position n in the synthesis strand is the first nucleotide of the predefined sequence to be incorporated into the first strand of the scaffold polynucleotide during the first ligation step, and pairs with the first nucleotide of the predefined sequence to be incorporated into the second strand of the scaffold polynucleotide during the second ligation step. The nucleotide at position n+1 in the synthesis strand is the second nucleotide of the predefined sequence to be incorporated into the first strand of the scaffold polynucleotide during the first ligation step, and pairs with the second nucleotide of the predefined sequence to be incorporated into the second strand of the scaffold polynucleotide during the second ligation step. Thus in such a method wherein x=1, two nucleotides of the predefined sequence will be incorporated into the first strand during the first ligation reaction.

In such a method wherein x=1 and the universal nucleotide occupies position n+2, the universal nucleotide is paired with a partner nucleotide in the helper strand which is also at position n+2 in the helper strand at the complementary ligation end.

In the first cleavage step (step 3) the synthesis strand of the scaffold polynucleotide is cleaved between positions n+1+x and n+x. Thus in a method wherein x=1 the synthesis strand of the scaffold polynucleotide is cleaved between positions n+2 and n+1.

In these particular further variants of synthesis method version 3 the value selected for x in the first cleavage and ligation steps must be at least 1. Accordingly, the universal nucleotide will occupy a position which is n+2 or more. Since the scaffold polynucleotide is cleaved between positions n+1+x and n+x, this means that in such a method at least two nucleotides of the predefined sequence are incorporated into the first strand during the first ligation step.

In the second ligation step (step 4) the complementary ligation end of the second polynucleotide ligation molecule is structured such that the universal nucleotide occupies a nucleotide position in the synthesis strand defined by the formula n+1+x and is paired with a partner nucleotide at the same position in the helper strand at the complementary ligation end. For example, if x=1 the universal nucleotide occupies nucleotide position n+2 in the synthesis strand. Position n+2 is the second nucleotide position in the synthesis strand relative to nucleotide position n in the direction distal to the complementary ligation end. The nucleotide at position n in the synthesis strand is the first nucleotide of the predefined sequence to be incorporated into the second strand of the scaffold polynucleotide during the second ligation step, and pairs with the first nucleotide of the predefined sequence to be incorporated into the first strand of the scaffold polynucleotide during the first ligation step.

The nucleotide at position n+1 in the synthesis strand is the second nucleotide of the predefined sequence to be incorporated into the second strand of the scaffold polynucleotide during the second ligation step, and pairs with the second nucleotide of the predefined sequence to be incorporated into the first strand of the scaffold polynucleotide during the first ligation step. Thus in such a method wherein x=1 in the second ligation and cleavage steps, two nucleotides of the predefined sequence will be incorporated into the second strand during the second ligation reaction.

In the second cleavage step (step 5) the synthesis strand of the scaffold polynucleotide is cleaved between positions n+1+x and n+x. Thus in a method wherein x=1 in the second ligation and cleavage steps the synthesis strand of the scaffold polynucleotide is cleaved between positions n+2 and n+1. In these particular further variants of synthesis method version 3 the value selected for x in the second ligation and cleavage steps must be at least 1. Accordingly, the universal nucleotide will occupy a position which is n+2 or more. Since the scaffold polynucleotide is cleaved between positions n+1+x and n+x, this means that in such a method at least two nucleotides of the predefined sequence are incorporated into the second strand during the second ligation step.

Thus the invention provides for a further variant method according to method of the invention version 3, wherein in step (2) the universal nucleotide is positioned in the synthesis strand of the first polynucleotide ligation molecule at a position defined by the formula n+1+x, and wherein in step (3) the ligated first strand of the scaffold polynucleotide is cleaved between positions n+1+x and n+x, wherein x is a whole number from 1 to 10 or more; and wherein in step (4) the universal nucleotide is positioned in the synthesis strand of the second polynucleotide ligation molecule at a position defined by the formula n+1+x, and wherein in step (5) the ligated second strand of the scaffold polynucleotide is cleaved between positions n+1+x and n+x, wherein x is a whole number from 1 to 10 or more; and wherein in steps (2) and (4) x is a number of nucleotide positions relative to position n in the direction distal to the complementary ligation end. In any of these further variant methods, the number selected for x in steps (2) and (3) and the number selected for x in steps (4) and (5) are chosen such that the number of nucleotides that are incorporated into the first strand of the scaffold polynucleotide in steps (2) and (3) may be the same as the number of nucleotides that are incorporated into the second strand of the scaffold polynucleotide in steps (4) and (5).

In any of these further variant methods, the number selected for x in steps (2) and (3) may have the same value as the number selected for x in steps (4) and (5).

According to this combination scheme the selection of 1 for the value of x in steps (2) and (3) and the selection of 1 for the value of x in steps (4) and (5) will give rise to a method which is defined as described for synthesis method of the invention version 7 (see FIG. 7) wherein two nucleotides of the predefined sequence are incorporated into the first strand during the first ligation reaction and two corresponding nucleotides of the predefined sequence are incorporated into the second strand during the second ligation reaction, thus incorporating two new pairs of nucleotides into the scaffold polynucleotide.

Thus synthesis method of the invention version 7 can be defined as a variant of synthesis method of the invention version 3 when considered in the context of the formulae set out above. The selection of 2 for the value of x for the first and second ligation and cleavage steps will give rise to a method wherein three nucleotides of the predefined sequence are incorporated into the first strand during the first ligation reaction and three corresponding nucleotides of the predefined sequence are incorporated into the second strand during the second ligation reaction, thus incorporating three new pairs of nucleotides into the scaffold polynucleotide. The value selected for x can be increased in this manner by the user to incorporate progressively more pairs of nucleotides into the scaffold polynucleotide during each cycle of synthesis.

As will be apparent from the description of the aforementioned further variant methods of synthesis method of the invention version 3, of which synthesis method of the invention version 7 is an example, these schematics give rise to methods wherein both first and second strands are cleaved at positions between the position occupied by the universal nucleotide and the last nucleotide of the predefined sequence to be incorporated into a strand in the preceding ligation reaction. For example, in FIG. 7 depicting synthesis method of the invention version 7, in step (3) the first strand is cleaved between the universal nucleotide and the nucleotide depicted as G, which is the last nucleotide of the predefined sequence to be incorporated into the first strand in the preceding ligation reaction (step 2). However, yet further variant methods are envisaged wherein the cleavage mechanism can be varied. An example of such a yet further variant can be defined generically with reference to synthesis method of the invention version 8.

According to such a yet further variant method, in the second ligation step (step 4) the complementary ligation end of the second polynucleotide ligation molecule is structured such that the universal nucleotide occupies a nucleotide position in the synthesis strand defined by the formula n+1+x and is paired with a partner nucleotide at the same position in the helper strand at the complementary ligation end. In these methods x is a whole number from 2 to 10 or more and is a number of nucleotide positions relative to position n in the direction distal to the complementary ligation end. For example, if x=2 the universal nucleotide occupies nucleotide position n+3 in the synthesis strand. Position n+3 is the third nucleotide position in the synthesis strand relative to nucleotide position n in the direction distal to the complementary ligation end. In the second cleavage step (step 5) the synthesis strand of the scaffold polynucleotide is cleaved between positions n+x and n+x−1. Thus in a method wherein x=2 the synthesis strand of the scaffold polynucleotide is cleaved between positions n+2 and n+1 and the universal nucleotide occupies position n+3.

These schematics give rise to methods wherein the second strand is cleaved at a nucleotide position immediately after the position occupied by the last nucleotide of the predefined sequence to be incorporated into the strand second (in the direction proximal to the helper strand) in the preceding second ligation reaction step (4). Thus in these schematics the value x defines a variable which affects the position of the universal nucleotide relative to the cleavage site as well as the number of nucleotides of the predefined sequence which are incorporated into the first and second strands.

Thus the invention provides for a yet further variant method as described above, wherein in step (4) the universal nucleotide is positioned in the synthesis strand of the second polynucleotide ligation molecule at a position defined by the formula n+1+x, wherein in step (5) the ligated second strand of the scaffold polynucleotide is cleaved between positions n+x and n+x−1, wherein x is a number of nucleotide positions relative to position n in the direction distal to the complementary ligation end and wherein x is a whole number from 2 to 10 or more.

According to this combination scheme, in the second ligation step (4) and second cleavage step (5) the selection of 2 for the value of x will give rise to a method which is defined as described for synthesis method of the invention version 8 (see FIG. 8). Thus synthesis method of the invention version 8 can be defined as a variant of synthesis method of the invention version 7 when considered in the context of the formulae set out above.

Yet further variant methods are envisaged wherein the positioning of the universal nucleotide relative to the cleavage site in the first cleavage step can be varied independently, as described below.

According to such a yet further variant method, in the first ligation step (step 2) the complementary ligation end of the first polynucleotide ligation molecule is structured such that the universal nucleotide occupies a nucleotide position in the synthesis strand defined by the formula n+1+x and is paired with a partner nucleotide in the same position in the helper strand at the complementary ligation end. In these methods x is a whole number from 2 to 10 or more. For example, if x=2 the universal nucleotide occupies nucleotide position n+3 in the synthesis strand. Position n+3 is the third nucleotide position in the synthesis strand relative to nucleotide position n in the direction distal to the complementary ligation end. In the first cleavage step (step 3) the synthesis strand of the scaffold polynucleotide is cleaved between positions n+x and n+x−1. Thus in a method wherein x=2 the synthesis strand of the scaffold polynucleotide is cleaved between positions n+2 and n+1 and the universal nucleotide occupies position n+3. These schematics give rise to methods wherein the first strand is cleaved at a nucleotide position immediately after the position occupied by the last nucleotide of the predefined sequence to be incorporated into the first strand (in the direction proximal to the helper strand) in the preceding first ligation reaction step (2). Thus in these schematics the value x defines a variable which affects the position of the universal nucleotide relative to the cleavage site as well as the number of nucleotides of the predefined sequence which are incorporated into the first and second strands.

Thus the invention provides for a yet further variant method as described above, wherein in step (2) the universal nucleotide is positioned in the synthesis strand of the first polynucleotide ligation molecule at a position defined by the formula n+1+x, wherein in step (3) the ligated first strand of the scaffold polynucleotide is cleaved between positions n+x and n+x−1, wherein x is a number of nucleotide positions relative to position n in the direction distal to the complementary ligation end and wherein x is a whole number from 2 to 10 or more.

As will be apparent from the above description, in the first of the two yet further variants, of which synthesis method of the invention version 8, is an illustrative example, the positioning of the universal nucleotide relative to the cleavage site in the second cleavage step can be varied in different methods, and in combination the positioning of the universal nucleotide relative to the cleavage site in the first cleavage step can remain fixed. Conversely, in the second of the two yet further variants, the positioning of the universal nucleotide relative to the cleavage site in the first cleavage step can be varied in different methods, and in combination the positioning of the universal nucleotide relative to the cleavage site in the second cleavage step can remain fixed. Other yet further variant methods are envisaged wherein the positioning of the universal nucleotide relative to the cleavage site in the first and second cleavage steps can be varied in different methods in any combination. Thus the invention provides yet further variant methods as described above, wherein in step (2) the universal nucleotide is positioned in the synthesis strand of the first polynucleotide ligation molecule at a position defined by the formula n+1+x, wherein in step (3) the ligated first strand of the scaffold polynucleotide is cleaved between positions n+x and n+x−1, wherein x is a whole number from 2 to 10 or more; and in step (4) the universal nucleotide is positioned in the synthesis strand of the second polynucleotide ligation molecule at a position defined by the formula n+1+x, wherein in step (5) the ligated second strand of the scaffold polynucleotide is cleaved between positions n+x and n+x−1, wherein x is a whole number from 2 to 10 or more; and wherein in steps (2) and (4) x is a number of nucleotide positions relative to position n in the direction distal to the complementary ligation end.

In any of these methods, the number selected for x in steps (2) and (3) and the number selected for x in steps (4) and (5) are chosen such that the number of nucleotides that are incorporated into the first strand of the scaffold polynucleotide in steps (2) and (3) is the same as the number of nucleotides that are incorporated into the second strand of the scaffold polynucleotide in steps (4) and (5).

In any of these methods, the number selected for x in steps (2) and (3) may have the same value as the number selected for x in steps (4) and (5).

### Further Variants of Synthesis Method Version 5

Further variants of synthesis method of the invention version 5 are provided wherein the method is performed in the same way as synthesis method version 5 described above except for the variations described below. In these variant methods two or more nucleotides can be incorporated into the first strand during the first ligation reaction, and subsequently two or more corresponding nucleotides can be incorporated into the second strand during the second ligation reaction. These further variant methods can also be defined generically via formulae. Generic variant methods and two specific illustrative variant methods, synthesis method versions 9 and 10, are described below.

Synthesis method of the invention version 9 is an illustrative specific example of a further variant of synthesis method version 5 and which can be defined generically as follows.

In the first ligation step (step 2) the complementary ligation end of the first polynucleotide ligation molecule is structured such that the universal nucleotide occupies a nucleotide position in the synthesis strand defined by the formula n+1+x and is paired with a partner nucleotide at the same position in the helper strand at the complementary ligation end. For example, if x=1 the universal nucleotide occupies nucleotide position n+2 in the synthesis strand. Position n+2 is the second nucleotide position in the synthesis strand relative to nucleotide position n in the direction distal to the complementary ligation end. The nucleotide at position n in the synthesis strand is the first nucleotide of the predefined sequence to be incorporated into the first strand of the scaffold polynucleotide during the first ligation step, and pairs with the first nucleotide of the predefined sequence to be incorporated into the second strand of the scaffold polynucleotide during the second ligation step. The nucleotide at position n+1 in the synthesis strand is the second nucleotide of the predefined sequence to be incorporated into the first strand of the scaffold polynucleotide during the first ligation step, and pairs with the second nucleotide of the predefined sequence to be incorporated into the second strand of the scaffold polynucleotide during the second ligation step. Thus in such a method wherein x=1, two nucleotides of the predefined sequence will be incorporated into the first strand during the first ligation reaction.

In such a method wherein x=1 and the universal nucleotide occupies position n+2, the universal nucleotide is paired with a partner nucleotide in the helper strand which is also at position n+2 in the helper strand at the complementary ligation end.

In the first cleavage step (step 3) the synthesis strand of the scaffold polynucleotide is cleaved between positions n+1+x and n+x. Thus in a method wherein x=1 the synthesis strand of the scaffold polynucleotide is cleaved between positions n+2 and n+1.

In these particular further variants of synthesis method version 5 the value selected for x in the first cleavage and ligation steps must be at least 1. Accordingly, the universal nucleotide will occupy a position which is n+2 or more. Since the scaffold polynucleotide is cleaved between positions n+1+x and n+x, this means that in such a method at least two nucleotides of the predefined sequence are incorporated into the first strand during the first ligation step.

In the second ligation step (step 4) the complementary ligation end of the second polynucleotide ligation molecule is structured such that the universal nucleotide occupies a nucleotide position in the synthesis strand defined by the formula n+1+x and is paired with a partner nucleotide at the same position in the helper strand at the complementary ligation end. For example, if x=1 the universal nucleotide occupies nucleotide position n+2 in the synthesis strand. Position n+2 is the second nucleotide position in the synthesis strand relative to nucleotide position n in the direction distal to the complementary ligation end. The nucleotide at position n in the synthesis strand is the first nucleotide of the predefined sequence to be incorporated into the second strand of the scaffold polynucleotide during the second ligation step, and pairs with the first nucleotide of the predefined sequence to be incorporated into the first strand of the scaffold polynucleotide during the first ligation step.

The nucleotide at position n+1 in the synthesis strand is the second nucleotide of the predefined sequence to be incorporated into the second strand of the scaffold polynucleotide during the second ligation step, and pairs with the second nucleotide of the predefined sequence to be incorporated into the first strand of the scaffold polynucleotide during the first ligation step. Thus in such a method wherein x=1 in the second ligation and cleavage steps, two nucleotides of the predefined sequence will be incorporated into the second strand during the second ligation reaction.

In the second cleavage step (step 5) the synthesis strand of the scaffold polynucleotide is cleaved between positions n+1+x and n+x. Thus in a method wherein x=1 in the second ligation and cleavage steps the synthesis strand of the scaffold polynucleotide is cleaved between positions n+2 and n+1. In these particular further variants of synthesis method version 5 the value selected for x in the second ligation and cleavage steps must be at least 1. Accordingly, the universal nucleotide will occupy a position which is n+2 or more. Since the scaffold polynucleotide is cleaved between positions n+1+x and n+x, this means that in such a method at least two nucleotides of the predefined sequence are incorporated into the second strand during the second ligation step.

Thus the invention provides for a further variant method according to method of the invention version 5, wherein in step (2) the universal nucleotide is positioned in the synthesis strand of the first polynucleotide ligation molecule at a position defined by the formula n+1+x, and wherein in step (3) the ligated first strand of the scaffold polynucleotide is cleaved between positions n+1+x and n+x, wherein x is a whole number from 1 to 10 or more; and wherein in step (4) the universal nucleotide is positioned in the synthesis strand of the second polynucleotide ligation molecule at a position defined by the formula n+1+x, and wherein in step (5) the ligated second strand of the scaffold polynucleotide is cleaved between positions n+1+x and n+x, wherein x is a whole number from 1 to 10 or more; and wherein in steps (2) and (4) x is a number of nucleotide positions relative to position n in the direction distal to the complementary ligation end.

In any of these further variant methods, the number selected for x in steps (2) and (3) and the number selected for x in steps (4) and (5) are chosen such that the number of nucleotides that are incorporated into the first strand of the scaffold polynucleotide in steps (2) and (3) may be the same as the number of nucleotides that are incorporated into the second strand of the scaffold polynucleotide in steps (4) and (5).

In any of these further variant methods, the number selected for x in steps (2) and (3) may have the same value as the number selected for x in steps (4) and (5).

According to this combination scheme the selection of 1 for the value of x in steps (2) and (3) and the selection of 1 for the value of x in steps (4) and (5) will give rise to a method which is defined as described for synthesis method of the invention version 9 (see FIG. 9) wherein two nucleotides of the predefined sequence are incorporated into the first strand during the first ligation reaction and two corresponding nucleotides of the predefined sequence are incorporated into the second strand during the second ligation reaction, thus incorporating two new pairs of nucleotides into the scaffold polynucleotide. Thus synthesis method of the invention version 9 can be defined as a variant of synthesis method of the invention version 5 when considered in the context of the formulae set out above. The selection of 2 for the value of x for the first and second ligation and cleavage steps will give rise to a method wherein three nucleotides of the predefined sequence are incorporated into the first strand during the first ligation reaction and three corresponding nucleotides of the predefined sequence are incorporated into the second strand during the second ligation reaction, thus incorporating three new pairs of nucleotides into the scaffold polynucleotide. The value selected for x can be increased in this manner by the user to incorporate progressively more pairs of nucleotides into the scaffold polynucleotide during each cycle of synthesis.

As will be apparent from the description of the aforementioned further variant methods of synthesis method of the invention version 5, of which synthesis method of the invention version 9 is an example, these schematics give rise to methods wherein both first and second strands are cleaved at positions between the position occupied by the universal nucleotide and the last nucleotide of the predefined sequence to be incorporated into a strand in the preceding ligation reaction. For example, in FIG. 9 depicting synthesis method of the invention version 9, in step (3) the first strand is cleaved between the universal nucleotide and the nucleotide depicted as C, which is the last nucleotide of the predefined sequence to be incorporated into the first strand in the preceding ligation reaction (step 2). However, yet further variant methods are envisaged wherein the cleavage mechanism can be varied. An example of such a yet further variant can be defined generically with reference to synthesis method of the invention version 10.

According to such a yet further variant method, in the first ligation step (step 2) the complementary ligation end of the first polynucleotide ligation molecule is structured such that the universal nucleotide occupies a nucleotide position in the synthesis strand defined by the formula n+1+x and is paired with a partner nucleotide at the same position in the helper strand at the complementary ligation end. In these methods x is a whole number from 2 to 10 or more. For example, if x=2 the universal nucleotide occupies nucleotide position n+3 in the synthesis strand. Position n+3 is the third nucleotide position in the synthesis strand relative to nucleotide position n in the direction distal to the complementary ligation end. In the first cleavage step (step 3) the synthesis strand of the scaffold polynucleotide is cleaved between positions n+x and n+x−1. Thus in a method wherein x=2 the synthesis strand of the scaffold polynucleotide is cleaved between positions n+2 and n+1 and the universal nucleotide occupies position n+3.

These schematics give rise to methods wherein the first strand is cleaved at a nucleotide position immediately after the position occupied by the last nucleotide of the predefined sequence to be incorporated into the first strand (in the direction proximal to the helper strand) in the preceding first ligation reaction step (2). Thus in these schematics the value x defines a variable which affects the position of the universal nucleotide relative to the cleavage site as well as the number of nucleotides of the predefined sequence which are incorporated into the first and second strands.

Thus the invention provides for a yet further variant method as described above, wherein in step (2) the universal nucleotide is positioned in the synthesis strand of the first polynucleotide ligation molecule at a position defined by the formula n+1+x, wherein in step (3) the ligated first strand of the scaffold polynucleotide is cleaved between positions n+x and n+x−1, wherein x is a number of nucleotide positions relative to position n in the direction distal to the complementary ligation end and wherein x is a whole number from 2 to 10 or more.

According to this combination scheme, in the first ligation step (2) and first cleavage step (3) the selection of 2 for the value of x will give rise to a method which is defined as described for synthesis method of the invention version 10 (see FIG. 10). Thus synthesis method of the invention version 10 can be defined as a variant of synthesis method of the invention version 9 when considered in the context of the formulae set out above.

Yet further variant methods are envisaged wherein the positioning of the universal nucleotide relative to the cleavage site in the second cleavage step can be varied independently, as described below.

According to such a yet further variant method, in the second ligation step (step 4) the complementary ligation end of the second polynucleotide ligation molecule is structured such that the universal nucleotide occupies a nucleotide position in the synthesis strand defined by the formula n+1+x and is paired with a partner nucleotide in the same position in the helper strand at the complementary ligation end. In these methods x is a whole number from 2 to 10 or more. For example, if x=2 the universal nucleotide occupies nucleotide position n+3 in the synthesis strand. Position n+3 is the third nucleotide position in the synthesis strand relative to nucleotide position n in the direction distal to the complementary ligation end. In the second cleavage step (step 5) the synthesis strand of the scaffold polynucleotide is cleaved between positions n+x and n+x−1. Thus in a method wherein x=2 the synthesis strand of the scaffold polynucleotide is cleaved between positions n+2 and n+1 and the universal nucleotide occupies position n+3.

These schematics give rise to methods wherein the second strand is cleaved at a nucleotide position immediately after the position occupied by the last nucleotide of the predefined sequence to be incorporated into the second strand (in the direction proximal to the helper strand) in the preceding second ligation reaction step (4). Thus in these schematics the value x defines a variable which affects the position of the universal nucleotide relative to the cleavage site as well as the number of nucleotides of the predefined sequence which are incorporated into the second strand.

Thus the invention provides for a yet further variant method as described above, wherein in step (4) the universal nucleotide is positioned in the synthesis strand of the second polynucleotide ligation molecule at a position defined by the formula n+1+x, wherein in step (5) the ligated second strand of the scaffold polynucleotide is cleaved between positions n+x and n+x−1, wherein x is a number of nucleotide positions relative to position n in the direction distal to the complementary ligation end and wherein x is a whole number from 2 to 10 or more.

As will be apparent from the above description, in the first of the two yet further variants, of which synthesis method of the invention version 10, is an illustrative example, the positioning of the universal nucleotide relative to the cleavage site in the first cleavage step can be varied in different methods, and in combination the positioning of the universal nucleotide relative to the cleavage site in the second cleavage step can remain fixed. Conversely, in the second of the two yet further variants, the positioning of the universal nucleotide relative to the cleavage site in the second cleavage step can be varied in different methods, and in combination the positioning of the universal nucleotide relative to the cleavage site in the first cleavage step can remain fixed. Other yet further variant methods are envisaged wherein the positioning of the universal nucleotide relative to the cleavage site in the first and second cleavage steps can be varied in different methods in any combination. Thus the invention provides yet further variant methods as described above, wherein in step (2) the universal nucleotide is positioned in the synthesis strand of the first polynucleotide ligation molecule at a position defined by the formula n+1+x, wherein in step (3) the ligated first strand of the scaffold polynucleotide is cleaved between positions n+x and n+x−1, wherein x is a whole number from 2 to 10 or more; and in step (4) the universal nucleotide is positioned in the synthesis strand of the second polynucleotide ligation molecule at a position defined by the formula n+1+x, wherein in step (5) the ligated second strand of the scaffold polynucleotide is cleaved between positions n+x and n+x−1, wherein x is a whole number from 2 to 10 or more; and wherein in steps (2) and (4) x is a number of nucleotide positions relative to position n in the direction distal to the complementary ligation end.

In any of these methods, the number selected for x in steps (2) and (3) and the number selected for x in steps (4) and (5) are chosen such that the number of nucleotides that are incorporated into the first strand of the scaffold polynucleotide in steps (2) and (3) is the same as the number of nucleotides that are incorporated into the second strand of the scaffold polynucleotide in steps (4) and (5).

In any of these methods, the number selected for x in steps (2) and (3) may have the same value as the number selected for x in steps (4) and (5).

### EXAMPLES

The following Examples provide support for the methods for synthesising a polynucleotide or oligonucleotide according to the invention, as well as exemplary constructs used in the methods. The Examples do not limit the invention.

The following Examples describe synthesis methods according to reaction schemes which are related to but which are not within the scope of the synthesis methods according to the invention.

The following Examples demonstrate the ability to perform synthesis reactions which involve steps of addition of a nucleotide of a predefined sequence to the synthesis strand of a scaffold polynucleotide, cleavage of the scaffold polynucleotide at a cleavage site defined by a universal nucleotide and ligation of a polynucleotide ligation molecule which comprises a partner nucleotide for the added nucleotide of the predefined sequence as well as a new universal nucleotide for use in creating a cleavage site for use in the next cycle of synthesis. The methods of the present invention incorporate several of these steps in a modified manner. Thus the following Examples provide illustrative support for the methods of the invention defined herein.

In the following Examples, and in corresponding FIGS. 17 to 63, references to synthesis method “versions 1, 2 and 3” or “version 1, 2 or 3 chemistry” etc. are to be interpreted according to the reaction schematics set out respectively in FIGS. 11 to 15 and not according to the reaction schematics set out in any of FIGS. 1 to 10 or descriptions of the same herein.

In relation to these Figures and corresponding methods, a complete explanation of the structures referred to as a scaffold polynucleotide molecule, a support strand, a synthesis strand, a primer strand portion, a helper strand portion and a polynucleotide ligation molecule, and methods relating to the incorporation into scaffold polynucleotide molecules or nucleotides comprising reversible terminator groups are provided in international patent application publication WO2018/134616.

Thus details relating to relevant reaction conditions relevant to the examples below can be found at page 46 of WO2018/134616. Details relating to scaffold polynucleotides relevant to the examples below can be found at page 47 of WO2018/134616. Details relating to exemplary methods relevant to the examples below can be found at the section starting at page 88 of WO2018/134616. Details relating to a synthesis strand relevant to the examples below can be found at page 118 of WO2018/134616. Details relating to a helper strand relevant to the examples below can be found at page 118 of WO2018/134616. Details relating to a primer strand relevant to the examples below can be found at page 121 of WO2018/134616. Details relating to a support strand relevant to the examples below can be found at page 122 of WO2018/134616.

### Example 1. Synthesis in the Absence of a Helper Strand

This example describes the synthesis of polynucleotides using 4 steps: incorporation of 3′-O-modified dNTPs on partial double-stranded DNA, cleavage, ligation and deprotection, with the first step taking place opposite a universal nucleotide, in this particular case inosine.

### Step 1: Incorporation

The first step describes controlled addition of a 3′-O-protected single nucleotide to an oligonucleotide by enzymatic incorporation by DNA polymerase (FIG. 17a).

### Materials and Methods

### Materials

1. 3′-O-modified dNTPs were synthesised in-house according to the protocol described in PhD thesis Jian Wu: Molecular Engineering of Novel Nucleotide Analogues for DNA Sequencing by Synthesis, Columbia University, 2008. The protocol for synthesis is also described in the patent application publication: J. William Efcavitch, Juliesta E. Sylvester, Modified Template-Independent Enzymes for Polydeoxynucleotide Synthesis, Molecular Assemblies US2016/0108382A1.
2. Oligonucleotides were designed in house and obtained from Sigma-Aldrich (FIG. 17h). The stock solutions were prepared at a concentration of 100 μM.
3. Therminator IX DNA polymerase was used that has been engineered by New England BioLabs with enhanced ability to incorporate 3-O-modified dNTPs. However, any DNA polymerase that could incorporate modified dNTPs could be used.
   Two Types of Reversible Terminators were Tested:

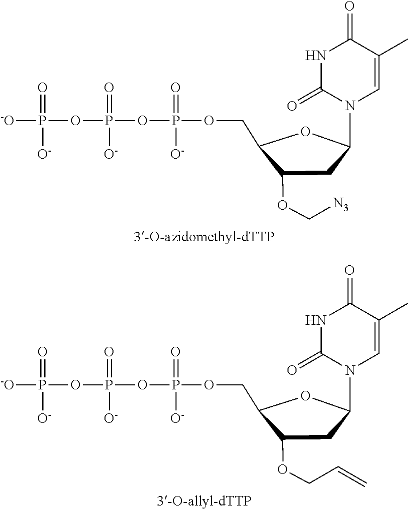

### Methods

1. 2 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs) was mixed with 12.25 μl of sterile deionized water (ELGA VEOLIA) in 1.5 ml Eppendorf tube.
2. 0.5 μl of 10 μM primer (synthesised strand) (5 pmol, 1 equiv) (SEQ ID NO: 1, FIG. 17h) and 0.75 μl of 10 μM template (support strand) (6 pmol, 1.5 equiv) (SEQ ID NO: 2, FIG. 17h) were added to the reaction mixture.
3. 3′-O-modified-dTTP (2 μl of 100 μM) and MnCl₂ (1 μl of 40 mM) were added.
4. 1.5 μl of Therminator IX DNA polymerase (15 U, New England BioLabs) was then added. However, any DNA polymerase that could incorporate modified dNTPs could be used.
5. The reaction was incubated for 20 minutes at 65° C.
6. The reaction was stopped by addition of TBE-Urea sample buffer (Novex).
7. The reaction was separated on polyacrylamide gel (15%) with TBE buffer and visualized by ChemiDoc MP imaging system (BioRad).

### Gel Electrophoresis and DNA Visualization:

1. 5 μl of reaction mixture was added to 5 μl of TBE-Urea sample buffer (Novex) in a sterile 1.5 ml Eppendorf tube and heated to 95° C. for 5 minutes using a heat ThermoMixer (Eppendorf).
2. 5 μl of the sample was then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 Amps for 40 minutes at room temperature.
4. The gel was visualized by ChemiDoc MP (BioRad) using Cy3 LEDS. Visualization and analysis was carried out on the Image lab 2.0 platform.

### Results

Customised engineered Therminator IX DNA polymerase from New England BioLabs is an efficient DNA polymerase able to incorporate 3′-O-modified-dNTPs opposite a universal nucleotide e.g. inosine (FIG. 17b-c).

Efficient incorporation opposite inosine occurred at a temperature of 65° C. (FIG. 17d-e).

Incorporation of 3′-O-modified-dTTPs opposite inosine requires the presence of Mn²⁺ ions (FIG. 17f-g). Successful conversion is marked in bold in FIGS. 17 c, e, g and h.

### Conclusion

Incorporation of 3-O-modified-dTTPs opposite inosine can be achieved with particularly high efficiency using customized engineered Therminator IX DNA polymerase from New England BioLabs, in the presence of Mn²⁺ ions and at a temperature at 65° C.

### Step 2: Cleavage

The second step describes a two-step cleavage of polynucleotides with either hAAG/Endo VIII or hAAG/chemical base (FIG. 18a).

### Materials and Methods

### Materials

1. Oligonucleotides utilized in Example 1 were designed in-house and synthesised by Sigma Aldrich (see table in FIG. 18(e) for sequences).
2. The oligonucleotides were diluted to a stock concentration of 100 uM using sterile distilled water (ELGA VEOLIA).

### Methods

A cleavage reaction on oligonucleotides was carried out using the procedure below:
1. A pipette (Gilson) was used to transfer 41 μl sterile distilled water (ELGA VEOLIA) into a 1.5 ml Eppendorf tube.
2. 50 of 10× ThermoPol® reaction buffer NEB (20 mM Tris-HCl, 10 mM (NH₄)₂SO₄, 10 mM KCl, 2 mM MgSO₄, 0.1% Triton® X-100, pH 8.8) were then added into the same Eppendorf tube.
3. 10 each of oligonucleotides (FIG. 18e); template (SEQ ID NO: 3) or any fluorescently tagged long oligo strand, primer with T (SEQ ID NO: 4) and control (SEQ ID NO: 5) all at 5 pmols were added into the same tube.
4. 10 of Human Alkyladenine DNA Glycosylase (hAAG) NEB (10 units/0) was added into the same tube.
5. Reaction mixture was then gently mixed by resuspension with a pipette, centrifuged at 13,000 rpm for 5 seconds and incubated at 37° C. for 1 hour.
6. Typically after incubation time had elapsed, the reaction was terminated by enzymatic heat inactivation (i.e. 65° C. for 20 minutes).

Purification under ambient conditions. The sample mixture was purified using the protocol outlined below:
1. 500 μl of buffer PNI QIAGEN (5M guanidinium chloride) was added to the sample and mixed by gentle resuspension with a pipette.
2. The mixture was transferred into a QIAquick spin column (QIAGEN) and centrifuged for 1 min at 6000 rpm.
3. After centrifugation, flow-through was discarded and 750 μl of buffer PE QIAGEN (10 mM Tris-HCl pH 7.5 and 80% ethanol) was added into the spin column and centrifuged for 1 min at 6000 rpm.
4. The flow-through was discarded and the spin column was centrifuged for an additional 1 min at 13000 rpm to remove residual PE buffer.
5. The spin column was then placed in a sterile 1.5 ml Eppendorf tube.
6. For DNA elution, 50 μl of Buffer EB QIAGEN (10 mM Tris.CL, pH 8.5) was added to the centre of the column membrane and left to stand for 1 min at room temperature.
7. The tube was then centrifuged at 13000 rpm for 1 minutes. Eluted DNA concentration was measured and stored at −20° C. for subsequent use.

### Measurement of Purified DNA Concentration was Determined Using the Protocol Below:

1. NanoDrop one (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop one was blanked by adding 2 μl of Buffer EB QIAGEN (10 mM Tris.CL, pH 8.5). Then step 2 was repeated after blanking.
4. DNA concentration was measured by adding 2 μl of the sample onto the pedestal and selecting the measure icon on the touch screen.

### Cleavage of the Generated Abasic Site was Carried Out Using the Procedure Below:

1. 2 μl (10-100 ng/0) DNA was added into a sterile 1.5 ml Eppendorf tube.
2. 40 μl (0.2M) NaOH or 1.5 μl Endo VIII NEB (10 units/0) and 5 μl 10× Reaction Buffer NEB (10 mM Tris-HCl, 75 mM NaCl, 1 mM EDTA, pH 8 @ 25° C.) was also added into the same tube and gently mixed by resuspension and centrifugation at 13000 rpm for 5 sec.
3. The resulting mixture was incubated at room temperature for 5 minutes for the NaOH treated sample while Endo VIII reaction mixture was incubated at 37° C. for 1 hr.
4. After incubation time had elapsed, the reaction mixture was purified using steps 1-7 of purification protocol as outlined above.

### Gel Electrophoresis and DNA Visualization:

1. 5 μl of DNA and TBE-Urea sample buffer (Novex) was added into a sterile 1.5 ml Eppendorf tube and heated to 95° C. for 2 minutes using a heat thermoblock (Eppendorf).
2. The DNA mixtures were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 Amps for 40 minutes at room temperature.
4. Detection and visualization of DNA in the gel was carried out with ChemiDoc MP (BioRad) using Cy3 LEDS. Visualization and analysis was carried out on the Image lab 2.0 platform.

### Results and Conclusion

The cleavage reaction without a helper strand showed a low percentage yield of cleaved to uncleaved DNA ratio of ˜7%:93% (FIG. 18b-d).

Cleavage results showed that in this specific example, and based on the specific reagents used, a low yield of cleaved DNA is obtained in the absence of a helper strand in comparison to the positive control. In addition the use of a chemical base for cleavage of the abasic site was less time-consuming compared to EndoVIII cleavage.

### Step 3: Ligation

The third step describes ligation of polynucleotides with DNA ligase in the absence of a helper strand. A diagrammatic illustration is shown in FIG. 19.

### Materials and Methods

### Materials

1. Oligonucleotides utilized in Example 1 were designed in-house and synthesised by Sigma Aldrich (see table in FIG. 19c for sequences).
2. The oligonucleotides were diluted to a stock concentration of 100 uM using sterile distilled water (ELGA VEOLIA).

### Methods

Ligation reaction on oligonucleotides was carried out using the procedure below:
1. A pipette (Gilson) was used to transfer 16 μl sterile distilled water (ELGA VEOLIA) into a 1.5 ml Eppendorf tube.
2. 100 of 2× Quick Ligation Reaction buffer NEB (132 mM Tris-HCl, 20 mM MgCl₂, 2 mM dithiothreitol, 2 mM ATP, 15% Polyethylene glycol (PEG6000) and pH 7.6 at 25° C.) was then added into the same Eppendorf tube.
3. 10 each of oligonucleotides (FIG. 19c); TAMRA or any fluorescently tagged phosphate strand (SEQ ID NO: 7), primer with T (SEQ ID NO: 8) and inosine strand (SEQ ID NO: 9), all at 5 pmols, was added into the same tube.
4. 10 of Quick T4 DNA Ligase NEB (400 units/μl) was added into the same tube.
5. The reaction mixture was then gently mixed by resuspension with a pipette, centrifuged at 13,000 rpm for 5 seconds and incubated at room temperature for 20 minutes.
6. Typically after incubation time had elapsed, reaction was terminated with the addition of TBE-Urea sample Buffer (Novex).
7. The reaction mixture was purified using the protocol outlined in purification steps 1-7 as described above.

Measurement of purified DNA concentration was determined using the protocol below:
1. NanoDrop one (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop one was blanked by adding 2 μl of Buffer EB QIAGEN (10 mM Tris.CL, pH 8.5), then step 2 was repeated after blanking.
4. DNA concentration was measured by adding 2 μl of the sample onto the pedestal and selecting the measure icon on the touch screen.
5. Purified DNA was run on a polyacrylamide gel and visualized in accordance with the procedure in steps 5-8 described above. No change in conditions or reagents was introduced.

### Results and Conclusion

In this specific example, and based on the specific reagents used, ligation of oligonucleotides with DNA ligase, in this particular case quick T4 DNA ligase, at room temperature (24° C.) in the absence of a helper strand results in a reduced amount of ligation product (FIG. 19b).

### Example 2. Version 1 Chemistry with a Helper Strand

This example describes the synthesis of polynucleotides using 4 steps: incorporation of 3′-O-modified dNTPs from a nick site, cleavage, ligation and deprotection, with the first step taking place opposite a universal nucleotide, in this particular case inosine. The method uses a helper strand which improves the efficiency of the ligation and cleavage steps.

### Step 1: Incorporation

The first step describes controlled addition of 3′-O-protected single nucleotide to oligonucleotide by enzymatic incorporation using DNA polymerase (FIG. 20a).

### Materials and Methods

### Materials

1. 3′-O-modified dNTPs were synthesised in-house according to the protocol described in PhD thesis Jian Wu: Molecular Engineering of Novel Nucleotide Analogues for DNA Sequencing by Synthesis. Columbia University, 2008. The protocol for synthesis is also described in the patent application publication: J. William Efcavitch, Juliesta E. Sylvester, Modified Template-Independent Enzymes for Polydeoxynucleotide Synthesis, Molecular Assemblies US2016/0108382A1.
2. Oligonucleotides were designed in house and obtained from Sigma-Aldrich. The stock solutions were prepared at a concentration of 100 μM. Oligonucleotides are shown in FIG. 20b.
3. Therminator IX DNA polymerase was used that has been engineered by New England BioLabs with enhanced ability to incorporate 3-O-modified dNTPs.

Two types of reversible terminators were tested:

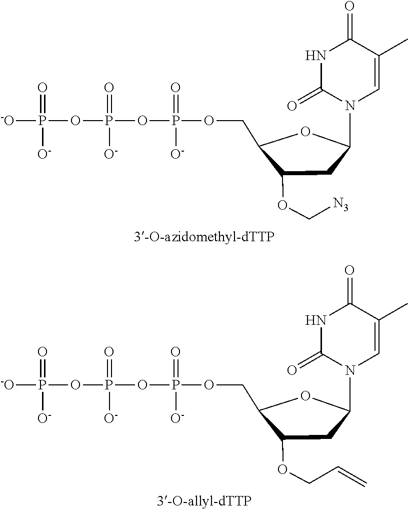

### Methods

1. 2 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs) was mixed with 10.25 μl of sterile deionized water (ELGA VEOLIA) in 1.5 ml Eppendorf tube.
2. 0.5 μl of 10 μM primer (5 pmol, 1 equiv) (SEQ ID NO: 10, Table in FIG. 20(b)), 0.75 μl of 10 μM template (6 pmol, 1.5 equiv) (SEQ ID NO: 11, Table in FIG. 20(b)), 2 μl of 10 μM of helper strand (SEQ ID NO: 12, Table in FIG. 20(b)) were added to the reaction mixture.
3. 3′-O-modified-dTTP (2 μl of 100 μM) and MnCl₂ (1 μl of 40 mM) were added.
4. 1.5 μl of Therminator IX DNA polymerase (15 U, New England BioLabs) was then added.
5. The reaction was incubated for 20 minutes at 65° C.
6. The reaction was stopped by addition of TBE-Urea sample buffer (Novex).
7. The reaction was separated on polyacrylamide gel (15%) TBE buffer and visualized by ChemiDoc MP imaging system (BioRad).

### Gel Electrophoresis and DNA Visualization:

1. 5 μl of reaction mixture was added to 5 μl of TBE-Urea sample buffer (Novex) in a sterile 1.5 ml Eppendorf tube and heated to 95° C. for 5 minutes using a heat ThermoMixer (Eppendorf).
2. 5 μl of the sample were then loaded into the wells of a 15% TBE-Urea gel 1.0 mm×10 well (Invitrogen) which contained preheated 1×TBE buffer Thermo Scientific (89 mM Tris, 89 mM Boric acid and 2 mM EDTA).
3. X-cell sure lock module (Novex) was fastened in place and electrophoresis performed at the following conditions; 260V, 90 Amps for 40 minutes at room temperature.
4. The gel was visualized by ChemiDoc MP (BioRad) using Cy3 LEDS. Visualization and analysis was carried out on the Image lab 2.0 platform.

The incorporation step can be studied according to the protocol described above.

### Step 2: Cleavage

The second step describes a two-step cleavage of polynucleotides with either hAAG/Endo VIII or hAAG/chemical base (×2) (FIG. 21a).

### Materials and Methods

### Materials

1. Oligonucleotides utilized in Example 2 were designed in-house and synthesised by Sigma Aldrich (see FIG. 21f for sequences).
2. The oligonucleotides were diluted to a stock concentration of 100 uM using sterile distilled water (ELGA VEOLIA).

### Methods

Cleavage reaction on oligonucleotides was carried out using the procedure below:
1. A pipette (Gilson) was used to transfer 41 μl sterile distilled water (ELGA VEOLIA) into a 1.5 ml Eppendorf tube.
2. 50 of 10× ThermoPol® Reaction buffer NEB (20 mM Tris-HCl, 10 mM (NH₄)₂SO₄, 10 mM KCl, 2 mM MgSO₄, 0.1% Triton® X-100, pH 8.8) was then added into the same Eppendorf tube.
3. 10 each of oligonucleotides (FIG. 21f); template (SEQ ID NO: 13) or any fluorescently tagged long oligo strand, primer with T (SEQ ID NO: 14), control (SEQ ID NO: 15) and helper strand (SEQ ID NO: 16), all at 5 pmols, were added into the same tube.
4. 10 of Human Alkyladenine DNA Glycosylase (hAAG) NEB (10 units/μl) was added into the same tube.
5. In the reaction using alternative base, 10 of Human Alkyladenine DNA Glycosylase (hAAG) NEB (100 units/μl) was added.
6. Reaction mixture was then gently mixed by resuspension with a pipette, centrifuged at 13,000 rpm for 5 seconds and incubated at 37° C. for 1 hour.
7. Typically after incubation time had elapsed, the reaction was terminated by enzymatic heat inactivation (i.e. 65° C. for 20 minutes).

Purification under ambient conditions. The sample mixture was purified using the protocol outlined below:
1. 500 μl of buffer PNI QIAGEN (5M guanidinium chloride) was added to the sample and mixed by gentle resuspension with a pipette.
2. The mixture was transferred into a QIAquick spin column (QIAGEN) and centrifuged for 1 min at 6000 rpm.
3. After centrifugation, flow-through was discarded and 750 μl of buffer PE QIAGEN (10 mM Tris-HCl pH 7.5 and 80% ethanol) was added into the spin column and centrifuged for 1 min at 6000 rpm.
4. The flow-through was discarded and the spin column was centrifuged for an additional 1 min at 13000 rpm to remove residual PE buffer.
5. The spin column was then placed in a sterile 1.5 ml Eppendorf tube.
6. For DNA elution, 50 μl of Buffer EB QIAGEN (10 mM Tris.CL, pH 8.5) was added to the centre of the column membrane and left to stand for 1 min at room temperature.
7. The tube was then centrifuged at 13000 rpm for 1 minute. Eluted DNA concentration was measured and stored at −20° C. for subsequent use.

Measurement of purified DNA concentration was determined using the protocol below:
1. NanoDrop one (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop one was blanked by adding 2 μl of Buffer EB QIAGEN (10 mM Tris.CL, pH 8.5). Then step 2 was repeated after blanking.
4. DNA concentration was measured by adding 2 μl of the sample onto the pedestal and selecting the measure icon on the touch screen.

Cleavage of generated abasic site was carried out using the procedure below:
1. 2 μl (10-100 ng/0) DNA was added into a sterile 1.5 ml Eppendorf tube.
2. 40 μl (0.2M) NaOH or 1.5 μl Endo VIII NEB (10 units/μl) and 5 μl 10×Reaction Buffer NEB (10 mM Tris-HCl, 75 mM NaCl, 1 mM EDTA, pH 8 @ 25° C.) was also added into the same tube and gently mixed by resuspension and centrifugation at 13000 rpm for 5 sec.
3. The resulting mixture was incubated at room temperature for 5 minutes for the 0.2 M NaOH treated sample while Endo VIII reaction mixture was incubated at 37° C. for 1 hr.
4. After incubation time had elapsed, the reaction mixture was purified using steps 1-7 of purification protocol as stated above.

Cleavage of generated abasic site using alternative basic chemical was carried out using the procedure below:
1. 1 μl (10-100 ng/μl) DNA was added into a sterile 1.5 ml Eppendorf tube. 2 μl of N,N′ dimethylethylenediamine Sigma (100 mM) which had been buffered at room temperature with acetic acid solution sigma (99.8%) to pH 7.4 was then added into the same tube.
2. 20 μl of sterile distilled water (ELGA VEOLIA) was added into the tube and gently mixed by resuspension and centrifugation at 13000 rpm for 5 sec.
3. The resulting mixture was incubated at 37° C. for 20 minutes.
4. After incubation time had elapsed, the reaction mixture was purified using steps 1-7 of the purification protocol stated above.

### Gel Electrophoresis and DNA Visualization:

1. 5 μl of DNA and TBE-Urea sample buffer (Novex) was added into a sterile 1.5 ml Eppendorf tube and heated to 95° C. for 2 minutes using a heat thermoblock (Eppendorf).
2. The DNA mixtures were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 Amps for 40 minutes at room temperature.
4. Detection and visualization of DNA in the gel was carried out with ChemiDoc MP (BioRad) using Cy3 LEDS. Visualization and analysis was carried out on the Image lab 2.0 platform.

### Results

Cleavage efficiency at a cleavage site comprising a universal nucleotide, in this particular case inosine, by hAAG DNA glycosylase was significantly increased from 10% in absence of helper strand to 50% in presence of helper strand (FIG. 21b). hAAG and Endonuclease VIII cleave inosine with lower efficiency (10%) than hAAG and NaOH (50%). Chemical cleavage using 0.2M NaOH was shown to be preferable for cleavage of AP sites than Endonuclease VIII in the described system using nicked DNA (FIG. 21c). Mild N,N′-dimethylethylenediamine at neutral pH has high efficiency to cleave abasic sites as 0.2M NaOH, and therefore it is preferable compared with Endonuclease VIII and NaOH (FIGS. 21d-e).

### Conclusion

Three methods were evaluated for cleavage of DNA containing inosine. One full enzymatic method—hAAG/Endonuclease VIII, and two methods combining chemical and enzymatic cleavage—hAAG/NaOH and hAAG/dimethylethylamine were studied for DNA cleavage in Example 2.

hAAG/NaOH results showed a much higher yield of cleaved DNA (50%) in the presence of a helper strand in comparison to the absence of a helper strand (10%). In these specific examples, and based on the specific reagents used, helper strands increase yield of DNA cleavage.

Enzymatic cleavage using Endonuclease VIII as a substitute for NaOH was less efficient (10%) compared to NaOH (50%) in the presence of a helper strand.

The inclusion of an alternative mild chemical base N,N′-dimethylethylenediamine led to high cleavage efficiency of AP sites, as efficient as for NaOH, and, together with addition of 10× hAAG enzyme, had a significant increase on cleaved DNA (see FIG. 21e).

### Step 3: Ligation

The third step describes ligation of polynucleotides with DNA ligase in the presence of a helper strand. A diagrammatic illustration is shown in FIG. 22a.

### Materials and Methods

### Materials

1. Oligonucleotides were designed in-house and synthesised by Sigma Aldrich (see FIG. 22d for sequences).
2. The oligonucleotides were diluted to a stock concentration of 100 uM using sterile distilled water (ELGA VEOLIA).

### Methods

Ligation reaction on oligonucleotides was carried out using the procedure below:
1. A pipette (Gilson) was used to transfer 160 sterile distilled water (ELGA VEOLIA) into a 1.5 ml Eppendorf tube.
2. 100 of 2× Quick Ligation Reaction buffer NEB (132 mM Tris-HCl, 20 mM MgCl₂, 2 mM dithiothreitol, 2 mM ATP, 15% Polyethylene glycol (PEG6000) and pH 7.6 at 25° C.) was then added into the same Eppendorf tube.
3. 10 each of oligonucleotides (FIG. 22d); TAMRA or any fluorescently tagged phosphate strand (SEQ ID NO: 18), primer with T (SEQ ID NO: 19) and inosine strand (SEQ ID NO: 20) and helper strand (SEQ ID NO: 21), all at of 5 pmols, was added into the same tube.
4. 10 of Quick T4 DNA Ligase NEB (400 units/μl) was added into the same tube.
5. Reaction mixture was then gently mixed by resuspension with a pipette, centrifuged at 13,000 rpm for 5 seconds and incubated at room temperature for 20 minutes.
6. Typically after incubation time had elapsed, reaction was terminated with the addition of TBE-Urea sample Buffer (Novex).
7. The reaction mixture was purified using the protocol outlined in purification steps 1-7 as described above.

Measurement of purified DNA concentration was determined using the protocol below:
1. NanoDrop one (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop one was blanked by adding 2 μl of Buffer EB QIAGEN (10 mM Tris.CL, pH 8.5). Then step 2 was repeated after blanking.
4. DNA concentration was measured by adding 2 μl of the sample onto the pedestal and selecting the measure icon on the touch screen.
5. Purified DNA was run on a polyacrylamide gel and visualized in accordance with the procedure in steps 5-8 above. No change in conditions or reagents was introduced.

### Results and Conclusion

In this specific example, and based on the specific reagents used, reduced ligation activity is observed in the absence of a helper strand (FIG. 22b), whereas ligation proceeds with high efficiency in presence of a helper strand (FIG. 22c) and the product is formed in high yield.

### Example 3. Version 2 Chemistry with a Helper Strand

This example describes the synthesis of polynucleotides using 4 steps: incorporation of 3′-O-modified dNTPs on partial double-stranded DNA; cleavage, ligation and deprotection with the first step of incorporation taking place opposite a naturally complementary nucleotide which is positioned in the support strand adjacent to a universal nucleotide, in this particular case inosine.

### Step 1: Incorporation

### Materials and Methods

### Materials

The first step describes controlled addition of 3′-O-protected single nucleotide to oligonucleotide by enzymatic incorporation by DNA polymerase (FIG. 23a).
1. 3′-O-modified dNTPs were synthesised in-house according to the protocol described in PhD thesis Jian Wu: Molecular Engineering of Novel Nucleotide Analogues for DNA Sequencing by Synthesis. Columbia University, 2008. The protocol for synthesis is also described in the patent application publication: J. William Efcavitch, Juliesta E. Sylvester, Modified Template-Independent Enzymes for Polydeoxynucleotide Synthesis, Molecular Assemblies US2016/0108382A1.
2. Oligonucleotides were designed in house and obtained from Sigma-Aldrich (FIG. 23j). The stock solutions are prepared in concentration of 100 μM.
3. Therminator IX DNA polymerase was used that has been engineered by New England BioLabs with enhanced ability to incorporate 3-O-modified dNTPs. 3′-O-azidomethyl reversible terminators of all dNTPs were tested independently for incorporation:

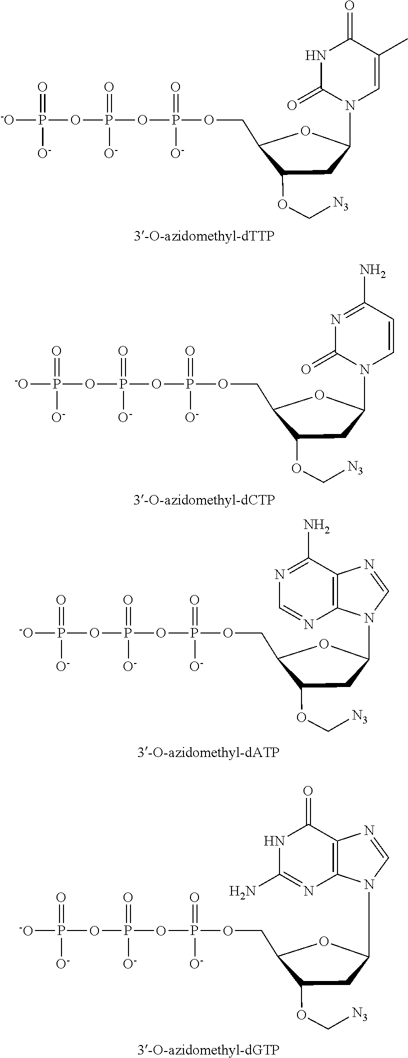

### Methods

1. 2 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs) was mixed with 12.25 μl of sterile deionized water (ELGA VEOLIA) in 1.5 ml Eppendorf tube.
2. 0.5 μl of 10 μM primer (5 pmol, 1 equiv) (SEQ ID NO: 22, FIG. 23j) and 0.75 μl of 10 μM template-A/G/T/C (6 pmol, 1.5 equiv) (SEQ ID NOS: 23 to 26, FIG. 23j) and 1 μl of 10 μM helper strand-T/C/A/G (10 pmol, 2 equiv) (SEQ ID NOS: 27 to 30, FIG. 23j) were added to the reaction mixture.
3. 3′-O-modified-dTTP/dCTP/dATP/dGTP (2 μl of 100 μM) and MnCl₂ (1 μl of 40 mM) were added.
4. 1.5 μl of Therminator IX DNA polymerase (15 U, New England BioLabs) was then added.
5. The reaction was incubated for 20 minutes at 65° C.
6. The reaction was stopped by addition of TBE-Urea sample buffer (Novex).
7. The reaction was separated on polyacrylamide gel (15%) TBE buffer and visualized by ChemiDoc MP imaging system (BioRad).

### Gel Electrophoresis and DNA Visualization:

1. 5 μl of reaction mixture was added to 5 μl of TBE-Urea sample buffer (Novex) in a sterile 1.5 ml Eppendorf tube and heated to 95° C. for 5 minutes using a heat ThermoMixer (Eppendorf).
2. 5 μl of the sample were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 Amps for 40 minutes at room temperature.
4. The gel was visualized by ChemiDoc MP (BioRad) using Cy3 LEDS. Visualization and analysis was carried out on the Image lab 2.0 platform.

### Results and Conclusions

Regarding the evaluation of the temperature on the incorporation of 3-O-azidomethyl-dTTP using Therminator IX DNA polymerase, the results indicate that incorporation of 3′-O-azidomethyl-dTTP in the presence of a helper strand for ligation goes to 90% after 5 minutes. 10% of primer remains unextended after 20 minutes at 37° C. and 47° C.

Therminator IX DNA polymerase at 2 mM Mn²⁺ ions and a temperature of 37° C. provide good conditions for incorporation of 3′-O-modified-dNTPs opposite a complementary base in DNA with high efficiency in the presence of the helper strand (from the ligation step from the previous cycle).

### Step 2: Cleavage

The second step describes a one-step cleavage of polynucleotides with Endonuclease V (FIG. 24a).

### Materials and Methods

### Materials

1. Oligonucleotides utilized in Example 3 were designed in-house and synthesised by Sigma Aldrich (see table in FIG. 24d for sequences).
2. The oligonucleotides were diluted to a stock concentration of 100 uM using sterile distilled water (ELGA VEOLIA).

### Methods

Cleavage reaction on oligonucleotides was carried out using the procedure below:
1. A pipette (Gilson) was used to transfer 41 μl sterile distilled water (ELGA VEOLIA) into a 1.5 ml Eppendorf tube.
2. 50 of 10× Reaction Buffer® NEB (50 mM Potassium Acetate, 20 mM Tris-acetate, 10 mM Magnesium Acetate, 1 mM DTT, pH 7.9@25° C.) was then added into the same Eppendorf tube.
3. 1 μl each of oligonucleotides (FIG. 24d); Template (SEQ ID NO: 31) or any fluorescently tagged long oligo strand, Primer with T (SEQ ID NO: 32) and control (SEQ ID NO: 33) and helper strand (SEQ ID NO: 34), all at 5 pmols, were added into the same tube.
4. 1 μl of Human Endonuclease V (Endo V) NEB (10 units/μl) was added into the same tube.
5. Reaction mixture was then gently mixed by resuspension with a pipette, centrifuged at 13,000 rpm for 5 seconds and incubated at 37° C. for 1 hour.
6. Typically after incubation time had elapsed, reaction was terminated by enzymatic heat inactivation (i.e. 65° C. for 20 minutes).

The sample mixture was purified using the protocol outlined below:
1. 500 μl of buffer PNI QIAGEN (5M guanidinium chloride) was added to the sample and mixed by gentle resuspension with a pipette.
2. The mixture was transferred into a QIAquick spin column (QIAGEN) and centrifuged for 1 min at 6000 rpm.
3. After centrifugation, flow-through was discarded and 750 μl of buffer PE QIAGEN (10 mM Tris-HCl pH 7.5 and 80% ethanol) was added into the spin column and centrifuged for 1 min at 6000 rpm.
4. The flow-through was discarded and the spin column was centrifuged for an additional 1 min at 13000 rpm to remove residual PE buffer.
5. The spin column was then placed in a sterile 1.5 ml Eppendorf tube.
6. For DNA elution, 50 μl of Buffer EB QIAGEN (10 mM Tris.CL, pH 8.5) was added to the centre of the column membrane and left to stand for 1 min at room temperature.
7. The tube was then centrifuged at 13000 rpm for 1 minutes. Eluted DNA concentration was measured and stored at −20° C. for subsequent use.

Measurement of purified DNA concentration was determined using the protocol below:
1. NanoDrop one (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop one was blanked by adding 2 μl of Buffer EB QIAGEN (10 mM Tris.CL, pH 8.5). Then step 2 was repeated after blanking.
4. DNA concentration was measured by adding 2 μl of the sample onto the pedestal and selecting the measure icon on the touch screen.

### Gel Electrophoresis and DNA Visualization:

1. 5 μl of DNA and TBE-Urea sample buffer (Novex) was added into a sterile 1.5 ml Eppendorf tube and heated to 95° C. for 2 minutes using a heat thermoblock (Eppendorf).
2. The DNA mixtures were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 Amps for 40 minutes at room temperature.
4. Detection and visualization of DNA in the gel was carried out with Chemidoc MP (BioRad) using Cy3 LEDS. Visualization and analysis was carried out on the Image lab 2.0 platform.

### Results and Conclusions

Cleavage results from Example 3 showed that a significantly high yield of cleaved DNA could be obtained with Endonuclease V in the presence or absence of the helper strand (FIG. 24c).

### Step 3: Ligation

The third step describes ligation of polynucleotides with DNA ligase in the presence of a helper strand. A diagrammatic illustration is shown in FIG. 25a.

### Materials and Methods

### Materials

1. Oligonucleotides utilized in Example 3 were designed in-house and synthesised by Sigma Aldrich (see table in FIG. 25b for sequences).
2. The oligonucleotides were diluted to a stock concentration of 100 uM using sterile distilled water (ELGA VEOLIA).

### Methods

Ligation reaction on oligonucleotides was carried out using the procedure below
1. A pipette (Gilson) was used to transfer 16 μl sterile distilled water (ELGA VEOLIA) into a 1.5 ml Eppendorf tube.
2. 10 μl of 2× Quick Ligation Reaction buffer NEB (132 mM Tris-HCl, 20 mM MgCl₂, 2 mM dithiothreitol, 2 mM ATP, 15% Polyethylene glycol (PEG6000) and pH 7.6 at 25° C.) was then added into the same Eppendorf tube.
3. 1 μl each of oligonucleotides (FIG. 25b); TAMRA or any fluorescently tagged phosphate strand (SEQ ID NO: 35), primer with T (SEQ ID NO: 36) and inosine strand (SEQ ID NO: 37) and helper strand (SEQ ID NO: 38) all having an amount of 5 pmols was added into the same tube.
4. 1 μl of Quick T4 DNA Ligase NEB (400 units/μl) was added into the same tube.
5. Reaction mixture was then gently mixed by resuspension with a pipette, centrifuged at 13,000 rpm for 5 seconds and incubated at room temperature for 20 minutes.
6. Typically after the incubation time had elapsed, the reaction was terminated with the addition of TBE-Urea sample Buffer (Novex).
7. The reaction mixture was purified using the protocol outlined in purification steps 1-7 as described above.

Measurement of purified DNA concentration was determined using the protocol below:
1. NanoDrop one (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop one was blanked by adding 2 μl of Buffer EB QIAGEN (10 mM Tris.CL, pH 8.5). Then step 2 was repeated after blanking.
4. DNA concentration was measured by adding 2 μl of the sample onto the pedestal and selecting the measure icon on the touch screen.
5. Purified DNA was run on a polyacrylamide gel and visualized in accordance with the procedure in steps 5-8 described above. No change in conditions or reagents was introduced.

### Gel Electrophoresis and DNA Visualization:

1. 5 μl of DNA and TBE-Urea sample buffer (Novex) was added into a sterile 1.5 ml Eppendorf tube and heated to 95° C. for 2 minutes using a heat thermoblock (Eppendorf).
2. The DNA mixtures were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 Amps for 40 minutes at room temperature.
4. Detection and visualization of DNA in the gel was carried out with ChemiDoc MP (BioRad) using Cy3 LEDS. Visualization and analysis was carried out on the Image lab 2.0 platform.

### Step 4: Deprotection

Deprotection step (FIG. 26a) was studied on DNA model bearing 3′-O-azidomethyl group that is introduced to DNA by incorporation of 3′-O-azidomethyl-dNTPs by Therminator IX DNA polymerase. Deprotection was carried out by tris(carboxyethyl)phosphine (TCEP) and monitored by extension reaction when mixture of all natural dNTPs is added to the solution of the purified deprotected DNA.

### Materials and Methods

### Materials

1. Oligonucleotides utilized in Example 3 were designed in-house and synthesised by Sigma Aldrich (see FIG. 26i for sequences).
2. The oligonucleotides were diluted to a stock concentration of 100 uM using sterile distilled water (ELGA VEOLIA).
3. Enzymes were purchased from New England BioLabs.

### Methods

1. 2 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs) was mixed with 12.25 μl of sterile deionized water (ELGA VEOLIA) in 1.5 ml Eppendorf tube.
2. 1 μl of 10 μM primer (10 pmol, 1 equiv) (SEQ ID NO: 39, FIG. 26i) and 1.5 μl of either 10 μM template-A/G/T/C (15 pmol, 1.5 equiv) (SEQ ID NOS: 40 to 43, FIG. 26i) were added to the reaction mixture.
3. 3′-O-modified-dTTP/dCTP/dATP/dGTP (2 μl of 100 μM) and MnCl₂ (1 μl of 40 mM) were added.
4. 1.5 μl of Therminator IX DNA polymerase (15 U, New England BioLabs) was then added.
5. The reaction was incubated for 5 minutes at 37° C.
6. 4 μL of the sample was taken out and mixed with 0.5 ul of 5 mM dNTP mix and allowed to react for 10 minutes for control reaction.
7. 40 μL of the 500 mM TCEP in 1M TRIS buffer pH 7.4 was added to the reaction mixture and allowed to react for 10 minutes at 37° C.
8. The reaction mixture was purified using QIAGEN Nucleotide removal kit eluting by 20 μL of 1× Thermopol® buffer.
9. 1 μL of 5 mM dNTP mix and 1 μL of DeepVent (exo-) DNA polymerase were added to the purified reaction mixture and allowed to react 10 minutes.
10. The reaction was stopped by addition of TBE-Urea sample buffer (Novex).
11. The reaction was separated on polyacrylamide gel (15%) TBE buffer and visualized by ChemiDoc MP imaging system (BioRad).

### Results and Conclusion

50 mM TCEP was not sufficient to cleave 3′-O-azidomethyl group with high efficiency on 0.2 μM DNA model (FIG. 26h). In contrast, 300 mM TCEP successfully cleaved 3′-O-azidomethyl group with 95% efficiency on 0.2 μM DNA model (FIG. 26h).

### Example 4. Version 2 Chemistry with Double Hairpin Model

This Example describes the synthesis of polynucleotides using 4 steps on a two-hairpin model: incorporation of 3′-O-modified dNTPs from a nick site; cleavage, ligation and deprotection with the first step taking place opposite a naturally complementary nucleotide which is positioned in the support strand adjacent to a universal nucleotide, in this particular case inosine.

### Step 1: Incorporation

The first step describes controlled addition of 3′-O-protected single nucleotide to oligonucleotide by enzymatic incorporation by DNA polymerase (FIG. 27a).

### Materials and Methods

### Materials

1. 3′-O-modified dNTPs were synthesised in-house according to the protocol described in PhD thesis Jian Wu: Molecular Engineering of Novel Nucleotide Analogues for DNA Sequencing by Synthesis. Columbia University, 2008. The protocol for synthesis is also described in the patent application publication: J. William Efcavitch, Juliesta E. Sylvester, Modified Template-Independent Enzymes for Polydeoxynucleotide Synthesis, Molecular Assemblies US2016/0108382A1.
2. Oligonucleotides were designed in house and obtained from Sigma-Aldrich (FIG. 27c). The stock solutions were prepared in concentration of 100 μM.
3. Therminator IX DNA polymerase was used that has been engineered by New England BioLabs with enhanced ability to incorporate 3-O-modified dNTPs.

### 3′-O-Azidomethyl-dTTP was Tested for Incorporation:

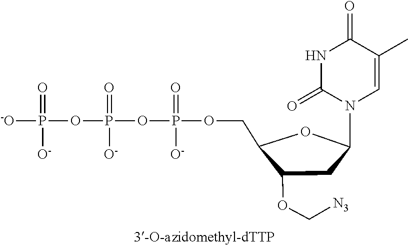

### Method

1. 2 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs) was mixed with 10.25 μl of sterile deionized water (ELGA VEOLIA) in 1.5 ml Eppendorf tube.
2. 0.5 μl of 10 μM hairpin oligonucleotide (5 pmol, 1 equiv) (SEQ ID NO: 44, FIG. 27c) was added to the reaction mixture.
3. 3′-O-modified-dTTP (2 μl of 100 μM) and MnCl₂ (1 μl of 40 mM) were added.
4. 1.5 μl of Therminator IX DNA polymerase (15 U, New England BioLabs) was then added.
5. The reaction was incubated for 20 minutes at 65° C.
6. The reaction was stopped by addition of TBE-Urea sample buffer (Novex).
7. The reaction was separated on polyacrylamide gel (15%) TBE buffer and visualized by ChemiDoc MP imaging system (BioRad).

### Gel Electrophoresis and DNA Visualization:

1. 5 μl of reaction mixture was added to 5 μl of TBE-Urea sample buffer (Novex) in a sterile 1.5 ml Eppendorf tube and heated to 95° C. for 5 minutes using a heat ThermoMixer (Eppendorf).
2. 5 μl of the sample were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 Amps for 40 minutes at room temperature.
4. The gel was visualized by ChemiDoc MP (BioRad) using Cy3 LEDS. Visualization and analysis was carried out on the Image lab 2.0 platform.

### Results

DNA polymerases incorporate 3′-O-modified-dTTPs opposite its naturally complementary base in a hairpin construct.

### Step 2: Cleavage

The second step describes a one-step cleavage of a hairpin model in this particular case with Endonuclease V (FIG. 28a).

### Materials and Methods

Materials
1. Oligonucleotides utilized in Example 4 were designed in-house and synthesised by Sigma Aldrich (see FIG. 28c for sequences).
2. The oligonucleotides were diluted to a stock concentration of 100 uM using sterile distilled water (ELGA VEOLIA).

### Methods

Cleavage reaction on hairpin oligonucleotides was carried out using the procedure below:
1. A pipette (Gilson) was used to transfer 43 μl sterile distilled water (ELGA VEOLIA) into a 1.5 ml Eppendorf tube.
2. 50 of 10× Reaction Buffer® NEB (50 mM potassium acetate, 20 mM Tris-acetate, 10 mM magnesium acetate, 1 mM DTT, pH 7.9@25° C.) was then added into the same Eppendorf tube.
3. 111 of hairpin oligonucleotide (SEQ ID NO: 45, FIG. 28c) having an amount of 5 pmols was added into the same tube.
4. 10 of Human Endonuclease V (Endo V) NEB (30 units/μl) was added into the same tube.
5. The reaction mixture was then gently mixed by resuspension with a pipette, centrifuged at 13,000 rpm for 5 seconds and incubated at 37° C. for 1 hour.
6. Typically after incubation time had elapsed, the reaction was terminated by enzymatic heat inactivation (i.e. 65° C. for 20 minutes).

The sample mixture was purified using the protocol outlined below:
1. 500 μl of buffer PNI QIAGEN (5M guanidinium chloride) was added to the sample and mixed by gentle resuspension with a pipette.
2. The mixture was transferred into a QIAquick spin column (QIAGEN) and centrifuged for 1 min at 6000 rpm.
3. After centrifugation, flow-through was discarded and 750 μl of buffer PE QIAGEN (10 mM Tris-HCl pH 7.5 and 80% ethanol) was added into the spin column and centrifuged for 1 min at 6000 rpm.
4. The flow-through was discarded and the spin column was centrifuged for an additional 1 min at 13000 rpm to remove residual PE buffer.
5. The spin column was then placed in a sterile 1.5 ml Eppendorf tube.
6. For DNA elution, 50 μl of Buffer EB QIAGEN (10 mM Tris.CL, pH 8.5) was added to the centre of the column membrane and left to stand for 1 min at room temperature.
7. The tube was then centrifuged at 13000 rpm for 1 minute. Eluted DNA concentration was measured and stored at −20° C. for subsequent use.

Measurement of purified DNA concentration was determined using the protocol below:
1. NanoDrop One (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop One was blanked by adding 2 μl of Buffer EB QIAGEN (10 mM Tris.CL, pH 8.5). Then step 2 was repeated after blanking.
4. DNA concentration was measured by adding 2 μl of the sample onto the pedestal and selecting the measure icon on the touch screen.

### Gel Electrophoresis and DNA Visualization:

1. 5 μl of DNA and TBE-Urea sample buffer (Novex) was added into a sterile 1.5 ml Eppendorf tube and heated to 95° C. for 2 minutes using a heat ThermoMixer (Eppendorf).
2. The DNA mixtures were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 Amps for 40 minutes at room temperature.
4. Detection and visualization of DNA in the gel was carried out with ChemiDoc MP (BioRad) using Cy3 LEDS. Visualization and analysis was carried out on the Image lab 2.0 platform.

### Results and Conclusion

Cleavage results from Example 4 showed that a significantly high yield of digested hairpin DNA was obtained with Endonuclease V at 37° C. (FIG. 28b).

### Step 3: Ligation

The third step describes ligation of a hairpin model with DNA ligase. Diagrammatic illustration is shown in FIG. 29a.

### Materials and Methods

### Materials

1. Oligonucleotides utilized in Example 4 were designed in-house and synthesised by Sigma Aldrich (see FIG. 29c for sequences).

The oligonucleotides were diluted to a stock concentration of 100 uM using sterile distilled water (ELGA VEOLIA).

### Method

Ligation reaction on oligonucleotides was carried out using the procedure below:
1. A pipette (Gilson) was used to transfer 10 (5 pmols) of TAMRA or any fluorescently tagged phosphate hairpin oligo (SEQ ID NO: 46) into a 1.5 ml Eppendorf tube.
2. 15 μl (100 pmols) of inosine-containing hairpin construct (SEQ ID NO: 47) was then added into the same tube and gently mixed by resuspension with a pipette for 3 seconds.
3. 40 μl of Blunt/TA DNA Ligase NEB (180 units/μl) was added into the same tube.
4. Reaction mixture was then gently mixed by resuspension with a pipette, centrifuged at 13,000 rpm for 5 seconds and incubated at room temperature for 20 minutes.
5. Typically after incubation time had elapsed, the reaction was terminated with the addition of TBE-Urea sample buffer (Novex).
6. The reaction mixture was purified using the protocol outlined in purification steps 1-7 above.

Measurement of purified DNA concentration was determined using the protocol below:
1. NanoDrop One (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop One was blanked by adding 2 μl of Buffer EB QIAGEN (10 mM Tris.CL, pH 8.5). Then step 2 was repeated after blanking.
4. DNA concentration was measured by adding 2 μl of the sample onto the pedestal and selecting the measure icon on the touch screen.
5. Purified DNA was run on a polyacrylamide gel and visualized in accordance with the procedure in steps 5-8 as described above. No change in conditions or reagents was introduced.

### Gel Electrophoresis and DNA Visualization.

1. 5 μl of DNA and TBE-Urea sample buffer (Novex) was added into a sterile 1.5 ml Eppendorf tube and heated to 95° C. for 2 minutes using a heat ThermoMixer (Eppendorf).
2. The DNA mixtures were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 Amps for 40 minutes at room temperature.
4. Detection and visualization of DNA in the gel was carried out with ChemiDoc MP (BioRad) using Cy3 LEDS. Visualization and analysis was carried out on the Image lab 2.0 platform.

### Results

Ligation of hairpin oligonucleotides with blunt/TA DNA ligase at room temperature (24° C.) in the presence of a helper strand resulted high yield of ligated product. Ligated hairpin oligonucleotide after 1 minute showed a high yield of ligated DNA product with a ratio of ˜85%. The ligated hairpin oligonucleotide after 2 minutes showed a high yield of ligated DNA with a ratio of ˜85%. The ligated hairpin oligonucleotide after 3 minutes showed a high yield of ligated DNA product with a ratio of ˜85%. The ligated hairpin oligonucleotide after 4 minutes showed a high yield of ligated DNA product with a ratio of ˜>85% (FIG. 29b).

### Example 5. Version 2 Chemistry—Complete Cycle on Double Hairpin Model

This Example describes the synthesis of polynucleotides using 4 steps on a double hairpin model: incorporation of 3′-O-modified dNTPs from the nick site; cleavage, ligation and deprotection with the first step taking place opposite a naturally complementary nucleotide which is positioned in the support strand adjacent to a universal nucleotide, in this particular case inosine. One end of the hairpin serves as an attachment anchor.

The method starts by controlled addition of a 3′-O-protected single nucleotide to an oligonucleotide by enzymatic incorporation by DNA polymerase followed by inosine cleavage, ligation and deprotection (FIG. 30a).

### Materials and Methods

### Materials

1. 3′-O-modified dNTPs were synthesised in-house according to the protocol described in PhD thesis Jian Wu: Molecular Engineering of Novel Nucleotide Analogues for DNA Sequencing by Synthesis. Columbia University, 2008. The protocol for synthesis is also described in the patent application publication: J. William Efcavitch, Juliesta E. Sylvester, Modified Template-Independent Enzymes for Polydeoxynucleotide Synthesis, Molecular Assemblies US2016/0108382A1.
2. Oligonucleotides were designed in house and obtained from Sigma-Aldrich (FIG. 30c). The stock solutions are prepared in concentration of 100 μM.
3. Therminator IX DNA polymerase was used that has been engineered by New England BioLabs with enhanced ability to incorporate 3-O-modified dNTPs.

### 3′-O-Azidomethyl-dTTP was Tested for Incorporation:

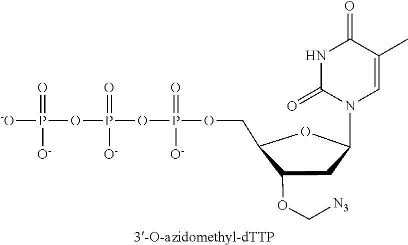

### Method

1. 2 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs) was mixed with 12.5 μl of sterile deionized water (ELGA VEOLIA) in 1.5 ml Eppendorf tube.
2. 2 μl of 10 μM double hairpin model oligonucleotide (20 pmol, 1 equiv) (SEQ ID NO: 48, FIG. 30c) were added to the reaction mixture.
3. 3′-O-modified-dTTP (2 μl of 100 μM) and MnCl₂ (1 μl of 40 mM) were added.
4. 1.5 μl of Therminator IX DNA polymerase (15 U, New England BioLabs) was then added.
5. The reaction was incubated for 10 minutes at 37° C.
6. The aliquot (5 μl) was taken out of the reaction mixture and 0.5 μl of natural dNTP mix was added and allowed to react for 10 minutes. The reaction was analysed by gel electrophoresis.
7. The reaction mixture was purified using the protocol outlined in purification steps 1-7.
8. The DNA sample was eluted by 20 μl of NEB reaction Buffer® (50 mM potassium acetate, 20 mM Tris-acetate, 10 mM magnesium acetate, 1 mM DTT, pH 7.9@25° C.) into clean Eppendorf tube.
9. 1 μl of Human Endonuclease V (Endo V) NEB (30 units/μl) was added into the same tube.
10. Reaction mixture was then gently mixed by resuspension with a pipette, centrifuged at 13,000 rpm for 5 seconds and incubated at 37° C. for 1 hour.
11. After incubation time had elapsed, reaction was terminated by enzymatic heat inactivation (i.e. 65° C. for 20 minutes).
12. The aliquot (5 μl) was taken out of the reaction mixture and analysed on polyacrylamide gel (15%) using TBE buffer and visualized by ChemiDoc MP imaging system (BioRad).
13. Reaction mixture was purified using the protocol outlined in purification steps 1-7 above.
14. The DNA sample was eluted by 20 μl of NEB Reaction Buffer® (50 mM potassium acetate, 20 mM Tris-acetate, 10 mM magnesium acetate, 1 mM DTT, pH 7.9@25° C.) into a clean Eppendorf tube.
15. 10 μl of 100 μM strand for ligation (1 nmol) (SEQ ID NO: 49, FIG. 30c) were added to the reaction mixture.
16. 400 of Blunt/TA DNA Ligase NEB (180 units/μl) was added into the purified DNA sample.
17. Reaction mixture was then gently mixed by resuspension with a pipette, centrifuged at 13,000 rpm for 5 seconds and incubated at room temperature for 20 minutes.
18. 40 μL of the 500 mM TCEP in 1M TRIS buffer pH 7.4 was added to the reaction mixture and allowed to react for 10 minutes at 37° C.
19. The reaction mixture was purified using QIAGEN nucleotide removal kit eluting by 20 μL of 1× Thermopol® buffer.

### Gel Electrophoresis and DNA Visualization:

1. 5 μl of reaction mixture was added to 5 μl of TBE-Urea sample buffer (Novex) in a sterile 1.5 ml Eppendorf tube and heated to 95° C. for 5 minutes using a heat ThermoMixer (Eppendorf).
2. 5 μl of the sample were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 Amps for 40 minutes at room temperature.
4. The gel was visualized by ChemiDoc MP (BioRad) using Cy3 LEDS. Visualization and analysis was carried out on the Image lab 2.0 platform.

Measurement of purified DNA concentration was determined using the protocol below:
1. NanoDrop One (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop One was blanked by adding 2 μl of Buffer EB QIAGEN (10 mM Tris.CL, pH 8.5). Then step 2 was repeated after blanking.
4. DNA concentration was measured by adding 2 μl of the sample onto the pedestal and selecting the measure icon on the touch screen.
5. Purified DNA was run on a polyacrylamide gel and visualized in accordance with the procedure in section 2 steps 5-8. No change in conditions or reagents was introduced.

The sample mixture was purified after each step using the protocol outlined below:
1. 500 μl of buffer PNI QIAGEN (5M guanidinium chloride) was added to the sample and mixed by gentle resuspension with a pipette.
2. The mixture was transferred into a QIAquick spin column (QIAGEN) and centrifuged for 1 min at 6000 rpm.
3. After centrifugation, flow-through was discarded and 750 μl of buffer PE QIAGEN (10 mM Tris-HCl pH 7.5 and 80% ethanol) was added into the spin column and centrifuged for 1 min at 6000 rpm.
4. The flow-through was discarded and the spin column was centrifuged for an additional 1 min at 13000 rpm to remove residual PE buffer.
5. The spin column was then placed in a sterile 1.5 ml Eppendorf tube.
6. For DNA elution, 20 μl of appropriate buffer for the reaction was added to the centre of the column membrane and left to stand for 1 min at room temperature.
7. The tube was then centrifuged at 13000 rpm for 1 minute. Eluted DNA concentration was measured and stored at −20° C. for subsequent use.

### Results

DNA polymerase incorporates 3′-O-modified-dTTPs opposite its naturally complementary base in a double hairpin construct (FIG. 30b).

### Example 6. Version 2 Chemistry—Complete Cycle on Single Hairpin Model Using Helper Strand

This Example describes the synthesis of polynucleotides using 4 steps on single-hairpin model: incorporation of 3′-O-modified dNTPs from nick site; cleavage, ligation and deprotection with the first step taking place opposite a naturally complementary base. The DNA synthesis uses a helper strand in the process.

The method starts by controlled addition of a 3′-O-protected single nucleotide to an oligonucleotide by enzymatic incorporation by DNA polymerase followed by inosine cleavage, ligation and deprotection (FIG. 31a).

### Materials and Methods

### Materials

1. 3′-O-modified dNTPs were synthesised in-house according to the protocol described in PhD thesis Jian Wu: Molecular Engineering of Novel Nucleotide Analogues for DNA Sequencing by Synthesis. Columbia University, 2008. The protocol for synthesis is also described in the patent application publication: J. William Efcavitch, Juliesta E. Sylvester, Modified Template-Independent Enzymes for Polydeoxynucleotide Synthesis, Molecular Assemblies US2016/0108382A1.
2. Oligonucleotides were designed in house and obtained from Sigma Aldrich (FIG. 31b). The stock solutions are prepared in concentration of 100 μM.
3. Therminator IX DNA polymerase was used that has been engineered by New England BioLabs with enhanced ability to incorporate 3-O-modified dNTPs.

### 3′-O-Azidomethyl-dTTP was Tested for Incorporation:

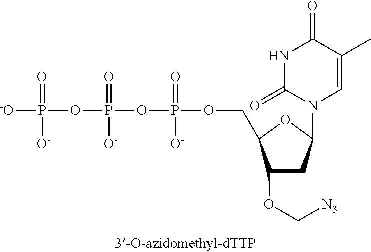

### Method

1. 2 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs) was mixed with 12.5 μl of sterile deionized water (ELGA VEOLIA) in 1.5 ml Eppendorf tube.
2. 2 μl of 10 μM Single hairpin model oligonucleotide (20 pmol, 1 equiv) (SEQ ID NO: 50, FIG. 31b) and Helper strand (30 pmol, 1.5 equiv) (SEQ ID NO: 51, FIG. 31b) were added to the reaction mixture.
3. 3′-O-modified-dTTP (2 μl of 100 μM) and MnCl₂ (1 μl of 40 mM) were added
4. 1.5 μl of Therminator IX DNA polymerase (15 U, New England BioLabs) was then added.
5. The reaction was incubated for 10 minutes at 37° C.
6. The aliquot (5 μl) was taken out of the reaction mixture and 0.5 μl of natural dNTP mix was added and allowed to react for 10 minutes. The reaction was analysed by gel electrophoresis.
7. The reaction mixture was purified using the protocol outlined in purification steps 1-7 above.
8. The DNA sample was eluted by 20 μl of NEB reaction Buffer® (50 mM potassium acetate, 20 mM Tris-acetate, 10 mM magnesium acetate, 1 mM DTT, pH 7.9@25° C.) into a clean Eppendorf tube.
9. 10 of Human Endonuclease V (Endo V) NEB (30 units/0) was added into the same tube.
10. Reaction mixture was then gently mixed by resuspension with a pipette, centrifuged at 13,000 rpm for 5 seconds and incubated at 37° C. for 1 hour.
11. After incubation time had elapsed, the reaction was terminated by enzymatic heat inactivation (i.e. 65° C. for 20 minutes).
12. The aliquot (5 μl) was taken out of the reaction mixture and analysed on polyacrylamide gel (15%) using TBE buffer and visualized by ChemiDoc MP imaging system (BioRad).
13. The reaction mixture was purified using the protocol outlined in purification steps 1-7 above.
14. The DNA sample was eluted by 20 μl of NEB reaction Buffer® (50 mM potassium acetate, 20 mM Tris-acetate, 10 mM magnesium acetate, 1 mM DTT, pH 7.9@25° C.) into clean Eppendorf tube.
15. 10 μl of 100 μM strand for ligation (1 nmol) (SEQ ID NO: 52, FIG. 31b) and 10 μl of 100 μM helper strand for ligation (1 nmol) (SEQ ID NO: 53, FIG. 31b) were added to the reaction mixture.
16. 40 μl of Blunt/TA DNA Ligase NEB (180 units/0) was added into the same tube.
17. Reaction mixture was then gently mixed by resuspension with a pipette, centrifuged at 13,000 rpm for 5 seconds and incubated at room temperature for 20 minutes.
18. 40 μl of the 500 mM TCEP in 1M TRIS buffer pH 7.4 was added to the reaction mixture and allowed to react for 10 minutes at 37° C.
19. The reaction mixture was purified using QIAGEN Nucleotide removal kit eluting by 20 μL of 1×NEB Thermopol® buffer.
20. Typically after incubation time had elapsed, reaction was terminated with the addition of TBE-Urea sample Buffer (Novex).

### Gel Electrophoresis and DNA Visualization:

1. 5 μl of reaction mixture was added to 5 μl of TBE-Urea sample buffer (Novex) in a sterile 1.5 ml Eppendorf tube and heated to 95° C. for 5 minutes using a heat ThermoMixer (Eppendorf).
2. 5 μl of the sample were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 amps for 40 minutes at room temperature.
4. The gel was visualized by ChemiDoc MP (BioRad) using Cy3 LEDS. Visualization and analysis was carried out on the Image lab 2.0 platform.

### Measurement of Purified DNA Concentration was Determined Using the Protocol Below:

1. NanoDrop One (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop One was blanked by adding 2 μl of Buffer EB QIAGEN (10 mM Tris.CL, pH 8.5). Then step 2 was repeated after blanking.
4. DNA concentration was measured by adding 2 μl of the sample unto the pedestal and selecting the measure icon on the touch screen.
5. Purified DNA was run on a polyacrylamide gel and visualized in accordance with the procedure noted above in steps 5-8. No change in conditions or reagents was introduced.

The sample mixture was purified after each step using the protocol outlined below:
1. 500 μl of buffer PNI QIAGEN (5M guanidinium chloride) was added to the sample and mixed by gentle resuspension with a pipette.
2. The mixture was transferred into a QIAquick spin column (QIAGEN) and centrifuged for 1 min at 6000 rpm.
3. After centrifugation, flow-through was discarded and 750 μl of buffer PE QIAGEN (10 mM Tris-HCl pH 7.5 and 80% ethanol) was added into the spin column and centrifuged for 1 min at 6000 rpm.
4. The flow-through was discarded and the spin column was centrifuged for an additional 1 min at 13000 rpm to remove residual PE buffer.
5. The spin column was then placed in a sterile 1.5 ml Eppendorf tube.
6. For DNA elution, 20 μl of appropriate buffer for the reaction was added to the centre of the column membrane and left to stand for 1 minute at room temperature.
7. The tube was then centrifuged at 13000 rpm for 1 minute. Eluted DNA concentration was measured and stored at −20° C. for subsequent use.

### Example 7. Version 3 Chemistry—Complete Cycle on Double Hairpin Model

This Example describes the synthesis of polynucleotides using 4 steps on a double-hairpin construct model: incorporation of 3′-O-modified dNTPs from the nick site; cleavage, ligation and deprotection with the first step taking place opposite a universal nucleotide, in this particular case an inosine base.

The method starts by controlled addition of a 3′-O-protected single nucleotide to an oligonucleotide by enzymatic incorporation by DNA polymerase followed by inosine cleavage, ligation and deprotection (FIG. 32a).

### Materials and Methods

### Materials

1. 3′-O-modified dNTPs were synthesised in-housed according to the protocol described in PhD thesis Jian Wu: Molecular Engineering of Novel Nucleotide Analogues for DNA Sequencing by Synthesis. Columbia University, 2008. The protocol for synthesis is also described in the patent application publication: J. William Efcavitch, Juliesta E. Sylvester, Modified Template-Independent Enzymes for Polydeoxynucleotide Synthesis, Molecular Assemblies US2016/0108382A1.
2. Oligonucleotides were designed in house and obtained from Sigma-Aldrich (FIG. 32b). The stock solutions are prepared in concentration of 100 μM.
3. Therminator IX DNA polymerase that has been engineered by New England BioLabs has enhanced ability to incorporate 3-O-modified dNTPs.

### 3′-O-Azidomethyl-dTTP was Tested for Incorporation:

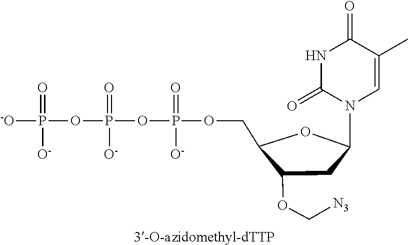

### Method

1. 2 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs) was mixed with 12.5 μl of sterile deionized water (ELGA VEOLIA) in 1.5 ml Eppendorf tube.
2. 2 μl of 10 μM double hairpin model oligonucleotide (20 pmol, 1 equiv) (SEQ ID NO: 54, FIG. 32b) were added to the reaction mixture.
3. 3′-O-modified-dTTP (2 μl of 100 μM) and MnCl₂ (1 μl of 40 mM) were added.
4. 1.5 μl of Therminator IX DNA polymerase (15 U, New England BioLabs) was then added.
5. The reaction was incubated for 10 minutes at 37° C.
6. The aliquot (5 μl) was taken out of the reaction mixture and 0.5 μl of natural dNTP mix was added and allowed to react for 10 minutes. The reaction was analysed by gel electrophoresis.
7. The reaction mixture was purified using the protocol outlined in purification steps 1-7.
8. The DNA sample was eluted by 20 μl of NEB Reaction Buffer® (50 mM potassium acetate, 20 mM Tris-acetate, 10 mM magnesium acetate, 1 mM DTT, pH 7.9@25° C.) into clean Eppendorf tube.
9. 1 μl of Human Endonuclease V (Endo V) NEB (30 units/μ1) was added into the same tube.
10. Reaction mixture was then gently mixed by resuspension with a pipette, centrifuged at 13,000 rpm for 5 seconds and incubated at 37° C. for 1 hour.
11. After the incubation time had elapsed, the reaction was terminated by enzymatic heat inactivation (i.e. 65° C. for 20 minutes).
12. The aliquot (5 μl) was taken out of the reaction mixture and analysed on polyacrylamide gel (15%) using TBE buffer and visualized by ChemiDoc MP imaging system (BioRad).
13. Reaction mixture was purified using the protocol outlined in purification steps 1-7 above.
14. The DNA sample was eluted by 20 μl of NEB Reaction Buffer® (50 mM potassium acetate, 20 mM Tris-acetate, 10 mM magnesium acetate, 1 mM DTT, pH 7.9@25° C.) into a clean Eppendorf tube.
15. 10 μl of 100 μM strand for ligation (1 nmol) (SEQ ID NO: 55, FIG. 32b), were added to the reaction mixture.
16. 40 μl of Blunt/TA DNA Ligase NEB (180 units/μ1) was added into the same tube.
17. Reaction mixture was then gently mixed by resuspension with a pipette, centrifuged at 13,000 rpm for 5 seconds and incubated at room temperature for 20 minutes.
18. 40 μL of the 500 mM TCEP in 1M TRIS buffer pH 7.4 was added to the reaction mixture and allowed to react for 10 minutes at 37° C.
19. The reaction mixture was purified using QIAGEN Nucleotide removal kit eluting by 20 μL of 1×NEB Thermopol® buffer.
20. Typically after incubation time had elapsed, reaction was terminated with the addition of TBE-Urea sample Buffer (Novex).

### Gel Electrophoresis and DNA Visualization:

1. 5 μl of reaction mixture was added to 5 μl of TBE-Urea sample buffer (Novex) in a sterile 1.5 ml Eppendorf tube and heated to 95° C. for 5 minutes using a heat ThermoMixer (Eppendorf).
2. 5 μl of the sample were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and electrophoresis performed at the following conditions; 260V, 90 amps for 40 minutes at room temperature.
4. The gel was visualized by ChemiDoc MP (BioRad) using Cy3 LEDS. Visualization and analysis was carried out on the Image lab 2.0 platform.

Measurement of purified DNA concentration was determined using the protocol below:
1. NanoDrop One (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop One was blanked by adding 2 μl of Buffer EB QIAGEN (10 mM Tris.CL, pH 8.5). Step 2 was then repeated after blanking.
4. DNA concentration was measured by adding 2 μl of the sample unto the pedestal and selecting the measure icon on the touch screen.
5. Purified DNA was run on a polyacrylamide gel and visualized in accordance with the procedure in section 2 steps 5-8. No change in conditions or reagents was introduced.

The sample mixture was purified after each step using the protocol outlined below:
1. 500 μl of buffer PNI QIAGEN (5M guanidinium chloride) was added to the sample and mixed by gentle resuspension with a pipette.
2. The mixture was transferred into a QIAquick spin column (QIAGEN) and centrifuged for 1 min at 6000 rpm.
3. After centrifugation, flow-through was discarded and 750 μl of buffer PE QIAGEN (10 mM Tris-HCl pH 7.5 and 80% ethanol) was added into the spin column and centrifuged for 1 min at 6000 rpm.
4. The flow-through was discarded and the spin column was centrifuged for an additional 1 min at 13000 rpm to remove residual PE buffer.
5. The spin column was then placed in a sterile 1.5 ml Eppendorf tube.
6. For DNA elution, 20 μl of appropriate buffer for the reaction was added to the centre of the column membrane and left to stand for 1 min at room temperature.
7. The tube was then centrifuged at 13000 rpm for 1 minutes. Eluted DNA concentration was measured and stored at −20° C. for subsequent use.

### Example 8. Version 2 Chemistry—Complete Two-Cycle Experiment on Double-Hairpin Model

This example describes a complete two-cycle experiment for the synthesis of polynucleotides using 4 steps on a double-hairpin model: incorporation of 3′-O-modified dNTPs from the nick site; deprotection, cleavage, and ligation with the first step taking place opposite a complementary base.

The method starts by controlled addition of a 3′-O-protected single nucleotide to an oligonucleotide by enzymatic incorporation by DNA polymerase followed by deprotection, inosine cleavage and ligation, as depicted in the reaction schematic for the first cycle shown in FIG. 33a. FIG. 33b shows a reaction schematic for the second cycle.

### Materials and Methods

### Materials

1. 3′-O-modified dNTPs were synthesised in-house according to the protocol described in PhD thesis Jian Wu: Molecular Engineering of Novel Nucleotide Analogues for DNA Sequencing by Synthesis. Columbia University, 2008. The protocol for synthesis is also described in the patent application publication: J. William Efcavitch, Juliesta E. Sylvester, Modified Template-Independent Enzymes for Polydeoxynucleotide Synthesis, Molecular Assemblies US2016/0108382A1.
2. Oligonucleotides were designed in house and obtained from Sigma-Aldrich (FIG. 33d). The stock solutions are prepared in concentration of 100 μM.
3. Therminator IX DNA polymerase that has been engineered by New England BioLabs has enhanced ability to incorporate 3′-O-modified dNTPs.
   3′-O-azidomethyl-dTTP and 3′-O-azidomethyl-dCTP were used for incorporation:

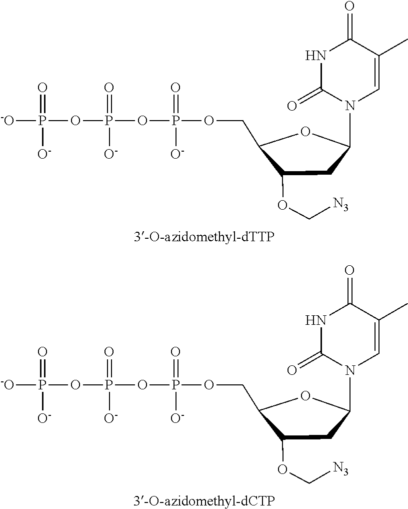

### Method

### 1^{st} Cycle:

1. 2 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs) was mixed with 12.5 μl of sterile deionized water (ELGA VEOLIA) in 1.5 ml Eppendorf tube.
2. 2 μl of 10 μM double hairpin model oligonucleotide (20 pmol, 1 equiv) (SEQ ID NO: 56, FIG. 33d) were added to the reaction mixture.
3. 3′-O-modified-dTTP (2 μl of 100 μM) and MnCl₂ (1 μl of 40 mM) were added.
4. 1.5 μl of Therminator IX DNA polymerase (15 U, New England BioLabs) was then added.
5. The reaction was incubated for 10 minutes at 37° C.
6. The aliquot (5 μl) was taken out of the reaction mixture and 0.5 μl of natural dNTP mix was added and allowed to react for 10 min. The reaction was analysed by gel electrophoresis.
7. 40 μL of the 500 mM TCEP in 1M TRIS buffer pH=7.4 was added to the reaction mixture and allowed to react for 10 minutes at 37° C.
8. The reaction mixture was purified using the protocol outlined in purification steps 1-7.
9. The DNA sample was eluted by 20 μl of NEB Reaction Buffer® (50 mM potassium acetate, 20 mM Tris-acetate, 10 mM magnesium acetate, 1 mM DTT, pH 7.9@25° C.) into a clean Eppendorf tube.
10. 10 of Human Endonuclease V (Endo V) NEB (30 units/0) was added into the same tube.
11. Reaction mixture was then gently mixed by resuspension with a pipette, centrifuged at 13,000 rpm for 5 seconds and incubated at 37° C. for 1 hour.
12. After incubation time had elapsed, the reaction was terminated by enzymatic heat inactivation (i.e. 65° C. for 20 mins).
13. The aliquot (5 μl) was taken out of the reaction mixture and analysed on polyacrylamide gel (15%) using TBE buffer and visualized by ChemiDoc MP imaging system (BioRad).
14. Reaction mixture was purified by QIAGEN Nucleotide Removal kit using the protocol outlined in purification steps 1-7.
15. The DNA sample was eluted by 20 μl of NEB Reaction Buffer® (50 mM potassium acetate, 20 mM Tris-acetate, 10 mM magnesium acetate, 1 mM DTT, pH 7.9@25° C.) into a clean Eppendorf tube.
16. 10 μl of 100 μM strand for ligation (1 nmol) (SEQ ID NO: 57, FIG. 33d), were added to the reaction mixture.
17. 40 μl of Blunt/TA DNA Ligase NEB (180 units/μl) was added into the same tube.
18. Reaction mixture was then gently mixed by resuspension with a pipette, centrifuged at 13,000 rpm for 5 seconds and incubated at room temperature for 20 mins.
19. Reaction mixture was purified by Streptavidin Magnetic Beads kit using the protocol outlined in purification steps 1-5.
20. Unligated oligonucleotide was digested by Lambda Exonuclease.
21. Reaction mixture was purified by QIAGEN Nucleotide Removal kit using the protocol outlined in purification steps 1-7.
22. The DNA sample was eluted by 20 μl of NEB Reaction Buffer® (50 mM potassium acetate, 20 mM Tris-acetate, 10 mM magnesium acetate, 1 mM DTT, pH 7.9@25° C.) into a clean Eppendorf tube.

### 2^{nd} Cycle:

23. 3′-O-modified-dCTP (2 μl of 100 μM) and MnCl₂ (1 μl of 40 mM) were added.
24. 1.5 μl of Therminator IX DNA polymerase (15 U, New England BioLabs) was then added.
25. The reaction was incubated for 10 minutes at 37° C.
26. The aliquot (5 μl) was taken out of the reaction mixture and 0.5 μl of natural dNTP mix was added and reacted for 10 min. The reaction was analysed by gel electrophoresis.
27. 40 μL of the 500 mM TCEP in 1M TRIS buffer pH=7.4 was added to the reaction mixture and reacted for 10 minutes at 37° C.
28. The reaction mixture was purified using the protocol outlined in purification steps 1-7.
29. The DNA sample was eluted by 20 μl of NEB Reaction Buffer® (50 mM potassium acetate, 20 mM Tris-acetate, 10 mM magnesium acetate, 1 mM DTT, pH 7.9@25° C.) into a clean Eppendorf tube.
30. 10 of Human Endonuclease V (Endo V) NEB (30 units/0) was added into the same tube.
31. The reaction mixture was then gently mixed by resuspension with a pipette, centrifuged at 13,000 rpm for 5 seconds and incubated at 37° C. for 1 hour.
32. After incubation time had elapsed, the reaction was terminated by enzymatic heat inactivation (i.e. 65° C. for 20 mins).
33. The aliquot (5 μl) was taken out of the reaction mixture and analysed on polyacrylamide gel (15%) using TBE buffer and visualized by ChemiDoc MP imaging system (BioRad).
34. The reaction mixture was purified using the protocol outlined in purification steps 1-7.
35. The DNA sample was eluted by 20 μl of NEB Reaction Buffer® (50 mM potassium acetate, 20 mM Tris-acetate, 10 mM magnesium acetate, 1 mM DTT, pH 7.9@25° C.) into clean Eppendorf tube.
36. 10 μl of 100 μM strand for ligation (1 nmol) (SEQ ID NO: 58, FIG. 33d), were added to the reaction mixture.
37. 40 μl of Blunt/TA DNA Ligase NEB (180 units/μ1) was added into the same tube.
38. Reaction mixture was then gently mixed by resuspension with a pipette, centrifuged at 13,000 rpm for 5 seconds and incubated at room temperature for 10 mins.
39. After incubation time had elapsed, the reaction was terminated with the addition of TBE-Urea sample Buffer (Novex).

### Gel Electrophoresis and DNA Visualization:

1. 5 μl of reaction mixture was added to 5 μl of TBE-Urea sample buffer (Novex) in a sterile 1.5 ml Eppendorf tube and heated to 95° C. for 5 mins using a heat ThermoMixer (Eppendorf).
2. 5 μl of the sample were then loaded into the wells of a 15% TBE-Urea gel 1.0 mm×10 well (Invitrogen) which contained preheated 1×TBE buffer Thermo Scientific (89 mM Tris, 89 mM boric acid and 2 mM EDTA).
3. X-cell sure lock module (Novex) was fastened in place and subjected to electrophoresis by applying the following conditions; 260V, 90 amps for 40 mins at room temperature.
4. The gel was visualized by ChemiDoc MP (BioRad) using Cy3 LEDS. Visualization and analysis was carried out on the Image lab 2.0 platform.

Measurement of purified DNA concentration was determined using the protocol below:
1. NanoDrop One (Thermo Scientific) was equilibrated by adding 2 μl of sterile distilled water (ELGA VEOLIA) onto the pedestal.
2. After equilibration, the water was gently wiped off using a lint-free lens cleaning tissue (Whatman).
3. NanoDrop One was blanked by adding 2 μl of Buffer EB QIAGEN (10 mM Tris.CL, pH 8.5). Then step 2 was repeated after blanking.
4. DNA concentration was measured by adding 2 μl of the sample unto the pedestal and selecting the measure icon on the touch screen.

The sample mixture was purified by QIAGEN Nucleotide Removal kit using the protocol outlined below:
1. 500 μl of buffer PNI QIAGEN (5M guanidinium chloride) was added to the sample and mixed by gentle resuspension with a pipette.
2. The mixture was transferred into a QIAquick spin column (QIAGEN) and centrifuged for 1 min at 6000 rpm.
3. After centrifugation, flow-through was discarded and 750 μl of buffer PE QIAGEN (10 mM Tris-HCl pH 7.5 and 80% ethanol) was added into the spin column and centrifuged for 1 min at 6000 rpm.
4. The flow-through was discarded and the spin column was centrifuged for an additional 1 min at 13000 rpm to remove residual PE buffer.
5. The spin column was then placed in a sterile 1.5 ml Eppendorf tube.
6. For DNA elution, 20 μl of appropriate buffer for the reaction was added to the centre of the column membrane and left to stand for 1 min at room temperature.
7. The tube was then centrifuged at 13000 rpm for 1 min.

After the ligation step, the sample mixture was purified using Streptavidin Magnetic Beads via the protocol outlined below:
1. 100 μl of Streptavidin Magnetic Beads (New England BioLabs) were washed 3 times by 200 μl of binding buffer (20 mM TRIS, 500 mM NaCl, pH=7.4).
2. Reaction mixture after ligation step is mixed with 10 volumes of binding buffer (20 mM TRIS, 500 mM NaCl, pH=7.4) and incubated with Streptavidin Magnetic Beads for 15 minutes at 20° C.
3. Streptavidin Magnetic Beads were washed 3 times by 200 μl of binding buffer (20 mM TRIS, 500 mM NaCl, pH=7.4).
4. Streptavidin Magnetic Beads were washed 3 times by deionized water.
5. The oligonucleotides were eluted by 40 μl of deionized water by heating to 95° C. for 3 minutes.

The results shown in FIG. 33c demonstrate the performance two complete synthesis cycles using an exemplary method of the invention.

### Example 9. Version 2 Chemistry—Complete Three-Cycle Experiment on Single-Hairpin Model

This example describes a complete three-cycle experiment for the synthesis of polynucleotides using 5 steps on a double-hairpin model: incorporation of 3′-O-modified dNTPs from the nick site, deprotection, cleavage, ligation and denaturation step with the first step taking place opposite a complementary base.

Exemplary schematic overviews of the method are shown in FIGS. 38, 39 and 40.

The method starts by the controlled addition of a 3′-O-protected single nucleotide to an oligonucleotide by enzymatic incorporation by DNA polymerase followed by deprotection, cleavage, ligation, and denaturation of the helper strand. FIG. 38 shows the 1st full cycle involving enzymatic incorporation, deprotection, cleavage, ligation and denaturation steps. In this example the oligonucleotide is extended by T nucleotide. FIG. 39 shows the 2nd full cycle following the 1st cycle involving enzymatic incorporation, deprotection, cleavage, ligation steps, and denaturation steps. In this example the oligonucleotide is extended by T nucleotide. FIG. 40 shows the 3rd full cycle following the 2nd cycle involving enzymatic incorporation, deprotection, cleavage, ligation, and denaturation steps. In this example the oligonucleotide is extended by T nucleotide.

### Materials and Methods

### Materials

1. 3′-O-modified dNTPs were synthesised in-house according to the protocol described in PhD thesis Jian Wu: Molecular Engineering of Novel Nucleotide Analogues for DNA Sequencing by Synthesis, Columbia University, 2008. The protocol for synthesis is also described in the patent application publication: J. William Efcavitch, Juliesta E. Sylvester, Modified Template-Independent Enzymes for Polydeoxynucleotide Synthesis, Molecular Assemblies US2016/0108382A1.
2. Oligonucleotides were designed in house and obtained from Integrated DNA Technologies, Sigma-Aldrich (FIG. 41). The stock solutions are prepared in concentration of 100 μM.
3. Therminator X DNA polymerase was used that has been engineered by New England BioLabs with enhanced ability to incorporate 3-O-modified dNTPs. Any DNA polymerase or other enzyme that could incorporate modified dNTPs could alternatively be used.

### 3′-O-Azidomethyl-dTTP was Used for Incorporation:

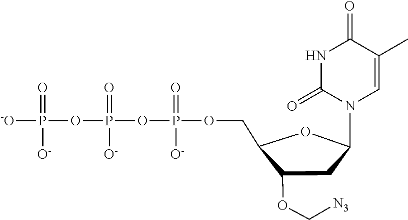

### Method

### 1^{St} Cycle:

1. 20 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs) and MnCl₂ solution (10 μl of 40 mM) were mixed with 139 μl of sterile deionized water (ELGA VEOLIA) in 1.5 ml Eppendorf tube.
2. 20 μl of 100 μM single hairpin model oligonucleotide (2 nmol, 1 equiv) (SEQ ID NO: 59, FIG. 41) was added to the reaction mixture.
3. The aliquot (4 μl) was taken out of the reaction mixture and 0.5 μl of natural dNTP mix (4 mM) and 0.5 μl of Bst DNA polymerase and 0.5 μl of Sulfolobus DNA polymerase IV were added and allowed to react for 10 min. The reaction was analysed by gel electrophoresis.
4. 3′-O-modified-dTTP (10 μl of 2 mM) was added.
5. 5 μl of Therminator X DNA polymerase (50 U, New England BioLabs) was then added. However, any DNA polymerase or other enzyme that could incorporate modified dNTPs could be used.
6. The reaction was incubated for 30 minutes at 37° C.
7. The reaction mixture was purified using QIAGEN Nucleotide Removal kit outlined in purification steps 66-72.
8. The DNA sample was eluted by 200 μl of TE buffer into a clean Eppendorf tube.
9. The aliquot (4 μl) was taken out of the reaction mixture and 0.5 μl of natural dNTP mix (4 mM) and 0.5 μl of Bst DNA polymerase and 0.5 μl of Sulfolobus DNA polymerase IV were added and allowed to react for 10 min. The reaction was analysed by gel electrophoresis.
10. 400 μL of the 500 mM TCEP was added to the reaction mixture and allowed to react for 10 minutes at 37° C.
11. The reaction mixture was purified using QIAGEN Nucleotide Removal kit outlined in purification steps 66-72.
12. The DNA sample was eluted by 150 μl of NEB Reaction Buffer® (50 mM potassium acetate, 20 mM Tris-acetate, 10 mM magnesium acetate, 1 mM DTT, pH 7.9@25° C.) into clean Eppendorf tube.
13. The aliquot (4 μl) was taken out of the reaction mixture and 0.5 μl of natural dNTP mix (4 mM) and 0.5 μl of Bst DNA polymerase and 0.5 μl of Sulfolobus DNA polymerase IV were added and allowed to react for 10 min. The reaction was analysed by gel electrophoresis.
14. 5 μl of Human Endonuclease V (Endo V) NEB (30 units/0) was added to the eluate and incubated at 37° C. for 30 minutes. Any suitable alternative endonuclease could be used.
15. After incubation time had elapsed, the reaction was terminated by enzymatic heat inactivation at 65° C. for 20 mins.
16. An aliquot (5 μl) was taken out of the reaction mixture and analysed on a polyacrylamide gel.
17. The reaction mixture was purified by QIAGEN Nucleotide Removal kit using the protocol outlined in purification steps 66-72.
18. The DNA sample was eluted by 100 μl of T3 DNA ligase buffer (2× concentrate) into a clean Eppendorf tube.
19. 20 μl of 100 μM inosine strand for ligation (2 nmol) and 20 μl of 100 μM helper strand for ligation (2 nmol) (SEQ ID NO: 60, 51, FIG. 41), and 40 μl of water were added to the reaction mixture.
20. 20 μl of T3 DNA Ligase NEB (3000 units/μ1) was added into the same tube (this could include any DNA ligating enzyme) and incubated at room temperature for 30 mins.

The reaction mixture was purified using the protocol for Streptavidin Magnetic Beads kit including the denaturation step outlined in purification steps 73-78.
21. The reaction mixture was purified using the protocol for QIAGEN Nucleotide Removal kit outlined in purification steps 66-72.
22. The DNA sample was eluted by 100 μl of TE buffer into a clean Eppendorf tube.

### 2^{nd} Cycle:

23. 15 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs), MnCl₂ solution (7.5 μl of 40 mM) and 19 μl of deionized water was added.
24. An aliquot (4 μl) was taken out of the reaction mixture and 0.5 μl of natural dNTP mix (4 mM) and 0.5 μl of Bst DNA polymerase and 0.5 μl of Sulfolobus DNA polymerase IV were added and allowed to react for 10 min. The reaction was analysed by gel electrophoresis.
25. 3′-O-modified-dTTP (7.5 μl of 2 mM) was added.
26. 5 μl of Therminator X DNA polymerase (50 U, New England BioLabs) was then added. Any DNA polymerase that could incorporate modified dNTPs could be used.
27. The reaction was incubated for 30 minutes at 37° C.
28. The reaction mixture was purified using QIAGEN Nucleotide Removal kit outlined in purification steps 66-72.
29. The DNA sample was eluted by 100 μl of TE buffer into a clean Eppendorf tube.
30. An aliquot (4 μl) was taken out of the reaction mixture and 0.5 μl of natural dNTP mix (4 mM) and 0.5 μl of Bst DNA polymerase and 0.5 μl of Sulfolobus DNA polymerase IV were added and allowed to react for 10 min. The reaction was analysed by gel electrophoresis.
31. 200 μL of the 500 mM TCEP was added to the reaction mixture and allowed to react for 10 minutes at 37° C.
32. The reaction mixture was purified using QIAGEN Nucleotide Removal kit outlined in purification steps 66-72.
33. The DNA sample was eluted by 100 μl of NEB Reaction Buffer® (50 mM potassium acetate, 20 mM Tris-acetate, 10 mM magnesium acetate, 1 mM DTT, pH 7.9@25° C.) into a clean Eppendorf tube.
34. The aliquot (4 μl) was taken out of the reaction mixture and 0.5 μl of natural dNTP mix (4 mM) and 0.5 μl of Bst DNA polymerase and 0.5 μl of Sulfolobus DNA polymerase IV were added and allowed to react for 10 min. The reaction was analysed by gel electrophoresis.
35. 5 μl of Human Endonuclease V (Endo V) NEB (30 units/0) was added to the eluate. and incubated at 37° C. for 30 minutes. Any suitable alternative endonuclease could be used.
36. After incubation time had elapsed, the reaction was terminated by enzymatic heat inactivation at 65° C. for 20 mins.
37. The aliquot (5 μl) was taken out of the reaction mixture and analysed on a polyacrylamide gel.
38. The reaction mixture was purified by QIAGEN Nucleotide Removal kit using the protocol outlined in purification steps 66-72.
39. The DNA sample was eluted by 60 μl of T3 DNA ligase buffer (2× concentrate) into a clean Eppendorf tube.
40. 20 μl of 100 μM inosine strand for ligation (2 nmol) and 20 μl of 100 μM helper strand for ligation (2 nmol) (SEQ ID NO: 60, 51, FIG. 41), and 10 μl of deionized water were added to the reaction mixture.
41. 10 μl of T3 DNA Ligase NEB (3000 units/μ1) was added into the same tube and incubated at room temperature for 30 mins. Any suitable DNA ligase could be used.
42. The reaction mixture was purified using the protocol for Streptavidin Magnetic Beads kit including denaturation step outlined in purification steps 73-78.
43. The reaction mixture was purified using the protocol for QIAGEN Nucleotide Removal kit outlined in purification steps 66-72.
44. The DNA sample was eluted by 46 μl of TE buffer into a clean Eppendorf tube.

### 3^{rd} Cycle:

45. 6 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England BioLabs), MnCl₂ solution (3 μl of 40 mM) was added.
46. An aliquot (4 μl) was taken out of the reaction mixture and 0.5 μl of natural dNTP mix (4 mM) and 0.5 μl of Bst DNA polymerase and 0.5 μl of Sulfolobus DNA polymerase IV were added and allowed to react for 10 min. The reaction was analysed by gel electrophoresis.
47. 3′-O-modified-dTTP (6 μl of 200 μM) was added.
48. 3 μl of Therminator X DNA polymerase (30 U, New England BioLabs) was then added. Any DNA polymerase or other suitable enzyme that could incorporate modified dNTPs could be used.
49. The reaction was incubated for 30 minutes at 37° C.
50. The reaction mixture was purified using QIAGEN Nucleotide Removal kit outlined in purification steps 66-72.
51. The DNA sample was eluted by 50 μl of TE buffer into a clean Eppendorf tube.
52. The aliquot (4 μl) was taken out of the reaction mixture and 0.5 μl of natural dNTP mix (4 mM) and 0.5 μl of Bst DNA polymerase and 0.5 μl of Sulfolobus DNA polymerase IV were added and allowed to react for 10 min. The reaction was analysed by gel electrophoresis.
53. 100 μL of the 500 mM TCEP was added to the reaction mixture and allowed to react for 10 minutes at 37° C.
54. The reaction mixture was purified using QIAGEN Nucleotide Removal kit outlined in purification steps 66-72.
55. The DNA sample was eluted by 49 μl of NEB Reaction Buffer® (50 mM potassium acetate, 20 mM Tris-acetate, 10 mM magnesium acetate, 1 mM DTT, pH 7.9@25° C.) into a clean Eppendorf tube.
56. An aliquot (4 μl) was taken out of the reaction mixture and 0.5 μl of natural dNTP mix (4 mM) and 0.5 μl of Bst DNA polymerase and 0.5 μl of Sulfolobus DNA polymerase IV were added and allowed to react for 10 min. The reaction was analysed by gel electrophoresis.
57. 5 μl of Human Endonuclease V (Endo V) NEB (30 units/μ1) was added to the eluate and incubated at 37° C. for 30 minutes. Any suitable endonuclease could alternatively be used.
58. After incubation time had elapsed, the reaction was terminated by enzymatic heat inactivation at 65° C. for 20 mins.
59. The aliquot (5 μl) was taken out of the reaction mixture and analysed on a polyacrylamide gel.
60. The reaction mixture was purified by QIAGEN Nucleotide Removal kit using the protocol outlined in purification steps 66-72.
61. The DNA sample was eluted by 30 μl of T3 DNA ligase buffer (2× concentrate) into a clean Eppendorf tube.
62. 10 μl of 100 μM inosine strand for ligation (2 nmol), 10 μl of 100 μM helper strand for ligation (2 nmol) (SEQ ID NO: 60, 51, FIG. 41) and 5 μl of water were added to the reaction mixture.
63. 5 μl of T3 DNA Ligase NEB (3000 units/μ1) was added into the same tube. (This could include any DNA ligating enzyme) and incubated at room temperature for 30 mins.
64. The reaction mixture was analysed by gel electrophoresis.

Purification of the reaction mixture by QIAGEN Nucleotide Removal kit after incorporation, deblock and cleavage steps using the protocol outlined below:
65. 10 volumes of buffer PNI QIAGEN (5M guanidinium chloride) was added to the sample and mixed by gentle resuspension with a pipette.
66. The mixture was transferred into a QIAquick spin column (QIAGEN) and centrifuged for 1 min at 6000 rpm.
67. After centrifugation, flow-through was discarded and 750 μl of buffer PE QIAGEN (10 mM Tris-HCl pH 7.5 and 80% ethanol) was added into the spin column and centrifuged for 1 min at 6000 rpm.
68. The flow-through was discarded and the spin column was centrifuged for an additional 1 min at 13000 rpm to remove residual PE buffer.
69. The spin column was then placed in a sterile 1.5 ml Eppendorf tube.
70. For DNA elution, 20-200 μl of appropriate buffer for the reaction was added to the centre of the column membrane and left to stand for 1 min at room temperature.
71. The tube was then centrifuged at 13000 rpm for 1 min.

Purification of the reaction after the ligation step using Streptavidin Magnetic Beads involving denaturation step was performed via the protocol outlined below:
72. 100 μl of Streptavidin Magnetic Beads (New England BioLabs) were washed 3 times by 200 μl of binding buffer (20 mM TRIS, 500 mM NaCl, pH=7.4).
73. Reaction mixture after ligation step is mixed with 10 volumes of binding buffer (20 mM TRIS, 500 mM NaCl, pH=7.4) and allowed to incubate with Streptavidin Magnetic Beads for 15 minutes at 20° C.
74. Streptavidin Magnetic Beads were washed 3 times by 200 μl of binding buffer (20 mM TRIS, 500 mM NaCl, pH=7.4).
75. To remove the helper strand, Streptavidin Magnetic Beads were heated to 80° C. in 200 μl of binding buffer (20 mM TRIS, 500 mM NaCl, pH=7.4), placed to magnet and supernatant was quickly discarded.
76. Streptavidin Magnetic Beads were washed 3 times with deionized water.
77. The oligonucleotides were eluted by 50-100 μl of deionized water by heating to 95° C. for 3 minutes.

### Results and Conclusion

FIG. 42 depicts a gel showing reaction products corresponding to a full three-cycle experiment comprising: incorporation, deblock, cleavage and ligation steps. The results shown demonstrate the performance of three complete synthesis cycles using an exemplary method of the invention.

### Example 10. Derivatization of a Polyacrylamide Surface and Subsequent Immobilisation of Molecules

This example describes the presentation of bromoacetyl groups on a polyacrylamide surface using N-(5-bromoacetamidylpentyl) acrylamide (BRAPA) and the subsequent surface immobilisation of thiolated molecules by their covalent coupling to bromoacetyl groups.

### Materials and Methods

Glass microscope slides and coverslips were cleaned by ultrasonication in acetone, ethanol and water sequentially for 10 mins each and dried with Argon. Clean glass coverslips were silanised with Trichloro(1H,1H,2H,2H-perfluorooctyl)silane in vapor phase in a polystyrene petri dish, sonicated twice in ethanol and dried with Ar (‘fluorinated coverslips’ hereafter). On glass microscope slides, 4% acrylamide/N,N′-Methylenebisacrylamide (19:1) solution was mixed with 100 μl of 10% (w/v) ammonium persulphate (APS), 10 μl of tetramethylethylenediamine (TEMED) spiked with N-(5-bromoacetamidylpentyl) acrylamide (BRAPA) at 0, 0.1, 0.2, and 0.3% (w/v) and quickly dispensed into a 4 mm diameter rubber gasket and subsequently sandwiched with a fluorinated coverslip with the fluorinated side facing towards the acrylamide solution and polymerised for 10 mins. After 10 mins, the surfaces were immersed in deionised water and left immersed for a total of 4 hrs, during which time the fluorinated coverslips were carefully removed. The polymerised polyacrylamide surfaces were dried with Argon.

The polyacrylamide surfaces were subsequently exposed to thiolated polyethylene glycol (1 kDa) fluorescein (FITC-PEG-SH), and carboxylated polyethylene glycol (1 kDa) fluorescein (FITC-PEG-COOH) as a negative control in sodium phosphate buffer (10 mM, pH 8) for 1 hr and subsequently washed sequentially with sodium phosphate buffer (10 mM, pH 7) and the same buffer containing 0.05% Tween20/0.5M NaCl to eliminate non-specifically adsorbed thiolated and carboxylated fluorophores. The surfaces were subsequently imaged by ChemiDoc (Bio-Rad) in the fluorescein channel.

### Results and Conclusion

FIG. 43 shows fluorescence signals and FIG. 44 shown measured fluorescence from polyacrylamide gel surfaces spiked with different amount of BRAPA exposed to FITC-PEG-SH and FITC-PEG-COOH. Immobilisation of fluorescein was only successful with polyacrylamide surfaces that were spiked with BRAPA and solely with thiolated fluorescein, with close to zero non-specific adsorption of the carboxylated fluorescein.

Significantly high positive fluorescence signals were obtained from polyacrylamide surfaces containing BRAPA (BRAPA 0.1, 0.2 and 0.3%) and only from thiolated molecules (FITC-PEG-SH) compared to those polyacrylamide surfaces without BRAPA (BRAPA 0%) and those polyacrylamide surfaces containing BRAPA and carboxylated molecules (FITC-PEG-COOH). The results indicate that specific covalent coupling has occurred between the bromoacetyl moiety from the surface and the thiol moiety from the fluorescein tagged molecules.

The results demonstrate that molecules, such as a molecule comprising a support strand and a synthesis strand for use in the methods of the present invention, can readily be immobilised on a surface substrate compatible with the polynucleotide synthesis reactions described herein.

### Example 11. Surface Immobilisation of Hairpin DNA Oligomers and Subsequent Incorporation of Fluorescently Labelled Deoxynucleoside Triphosphates

This example describes:

(1) a method of presenting bromoacetyl groups on a thin polyacrylamide surface;

(2) the subsequent immobilisation of hairpin DNA via covalent coupling of thiophosphate functionalised hairpin DNA with or without a linker; and

(3) the incorporation of 2′-deoxynucleotide triphosphate (dNTP) into hairpin DNA.

The method is compatible with virtually any type of material surface (e.g. metals, polymers etc).

(1): Fabrication of a Bromoacetyl Functionalised Thin Polyacrylamide Surface Materials and Methods

Glass microscope slides were first cleaned by ultrasonication in neat Decon 90 (30 mins), water (30 mins), 1M NaOH (15 mins), water (30 mins), 0.1M HCl (15 mins), water (30 mins) and finally dried with Argon.

2% (w/v) acrylamide monomer solution was first made by dissolving 1 g of acrylamide monomer in 50 ml of water. The acrylamide monomer solution was vortexed and degassed in argon for 15 mins. N-(5-bromoacetamidylpentyl) acrylamide (BRAPA, 82.5 mg) was dissolved in 825 μl of DMF and added to the acrylamide monomer solution and vortexed further. Finally, 1 ml of 5% (w/v) potassium persulphate (KPS) and 115 μl of neat tetramethylethylenediamine (TEMED) were added to the acrylamide solution, vortexed and the clean glass microscope slides were exposed to this acrylamide polymerisation mixture for 90 mins. After 90 mins, the surfaces were washed with deionised water and dried with argon. These surfaces will be referred to as ‘BRAPA modified surfaces’ in this example hereafter. As a negative control, polyacrylamide surfaces without BRAPA was also made in a similar manner as described above by excluding the addition of BRAPA solution into the acrylamide monomer solution. These surfaces will be referred to as ‘BRAPA control surface’ in this example hereafter.

(2): Covalent Coupling of Thiophosphate Functionalised Hairpin DNA onto Polyacrylamide Surfaces

### Materials and Methods

Rubber gaskets with a 4 mm diameter circular opening were placed and secured onto BRAPA modified and BRAPA control surfaces. The surfaces were first primed with sodium phosphate buffer (10 mM, pH 7) for 10 mins. The buffer was subsequently removed and the surfaces were exposed to 5′-fluorescently labelled (Alexa 647) hairpin DNA oligomers with and without a linker modified with six and single thiophosphates respectively at a 1 μM concentration and incubated for 1 hr in the dark. BRAPA modified surfaces were also incubated with DNA oligomers with and without linker but without thiophosphates as a control (referred to ‘oligomer control surfaces’ in this example hereafter). After incubation, the surfaces were rinsed in sodium phosphate (100 mM, pH 7) followed by Tris-EDTA buffer (10 mM Tris, 10 mM EDTA, pH 8) and finally with water. To remove any non-specifically adsorbed DNA oligomers, the surfaces were subsequently washed with water containing 1M sodium chloride and 0.05% (v/v) Tween20, washed with water and dried with argon. The surfaces were scanned on ChemiDoc imager in the Alexa 647 channel.

FIG. 45a shows the sequences of hairpin DNA without a linker immobilised on different samples. FIG. 45b shows the sequences of hairpin DNA with a linker immobilised on different samples.

### Results

Results are shown in FIGS. 46 and 47. FIG. 46 shows fluorescence signals originating from hairpin DNA oligomers with and without a linker immobilised onto bromoacetyl functionalised polyacrylamide surfaces, but not from BRAPA or oligomer controls.

FIG. 47 shows measured fluorescence intensity following DNA immobilisation on polyacrylamide surface. The Figure shows the surface fluorescence signals obtained from various polyacrylamide surfaces and shows that significantly higher signals were obtained from hairpin DNA oligomers immobilised onto BRAPA modified surfaces compared to BRAPA and oligomer control surfaces (as described in (2)), due to successful covalent immobilisation of DNA onto bromoacetyl functionalised polyacrylamide surfaces.

### Conclusion

Fluorescence signals from DNA were only prominently present from BRAPA modified surfaces that were spiked with BRAPA, indicative of successful covalent coupling of DNA onto the surface via the thiophosphate functionality. Homogenous and higher signals were obtained from DNA with the linker compared to DNA without the linker.

(3): Incorporation of Triphosphates into Hairpin DNA Oligomer with a Linker

### Materials and Methods

Rubber gaskets with a 9 mm diameter circular opening were placed on the BRAPA modified surfaces immobilised with the DNA oligomer with the linker and primed with incorporation buffer (50 mM TRIS pH 8, 1 mM EDTA, 6 mM MgSO₄, 0.05% tween20 and 2 mM MnCl₂) for 10 mins. The surfaces were subsequently exposed to incorporation buffer containing DNA polymerase (0.5 U/μl Therminator X DNA polymerase) and triphosphates (20 μM Alexa 488 labelled dUTP) and incubated for 1 hr (referred to as ‘polymerase surface’ in this example hereafter). Additional set of surfaces were also exposed to incorporation buffer without Therminator X DNA polymerase for 1 hr as a negative control (referred to as ‘negative surface’ in this example hereafter). After 1 hr, both types of sample were washed in water, subsequently exposed to water containing 1M sodium chloride and 0.05% (v/v) Tween20, and washed again with water. Fluorescence signals from the surfaces were measured using ChemiDoc in the Alexa 647 and Alexa 488 channels to monitor both the presence of hairpin DNA (Alexa 647) and incorporation of dUTP (Alexa 488).

### Results

FIG. 48 shows fluorescence signals detected from Alexa 647 and Alexa 488 channels before and after incorporation of Alexa 488-labelled dUTP. Unchanged positive signals from Alexa 647 before and after incorporation indicates that the surface immobilised hairpin DNA is stable during the incorporation reaction, while positive signals from Alexa 488 were only observed from the polymerase surfaces after incorporation reaction showing the successful incorporation of dUTPs only with the presence of polymerase.

FIG. 49 shows measured fluorescence signals in the Alexa 647 (hairpin DNA) and Alexa 488 (dUTP) channels obtained from ‘polymerase surfaces’ and ‘negative surfaces’ before and after incorporation of Alexa 488-labelled dUTP as described in (3). A significant increase in the Alexa 488 fluorescence signals was obtained after the incorporation reaction from the polymerase surface as a result of the successful incorporation, while the signals from negative surfaces remained the same after the incorporation reaction due to the absence of polymerase. Fluorescence signals in the Alexa 647 channel remained virtually unchanged after the incorporation reaction, indicating the presence of hairpin DNA on the surface. The slight reduction in the fluorescence signal may be attributed to the effect of photo-bleaching due to the second round of light exposure.

### Conclusion

The results demonstrate that a molecule comprising a support strand and a synthesis strand for use in the methods of the present invention, can readily be immobilised on a surface substrate compatible with the polynucleotide synthesis reactions described herein. The results further demonstrate that such a molecule can accept the incorporation of a new dNTP so as to extend the synthesis strand, whilst at the same time the molecule remains stable and attached to the substrate.

### Example 12. Cleavage and Ligation of Hairpin DNA Oligomers Immobilised to Derivatized Surfaces Via a Linker and Thiophosphate Covalent Linkage

This example describes the covalent coupling to derivatized surfaces of thiophosphate functionalised hairpin DNA with a linker, followed by cleavage and ligation reactions. The substrate preparation and coupling of hairpin DNA was carried out as described in Example 11.

(1): Cleavage of Immobilised Hairpin DNA Oligomers with a Linker

### Materials and Methods

Hairpin DNA was immobilised on surface BRAPA modified surfaces as described in Example 11. Four sets of triplicate surfaces including all the experimental controls for cleavage and ligation reactions were prepared. The experimental conditions are described in FIG. 50a. FIG. 50b shows the sequences of hairpin DNA immobilised on different samples.

After the DNA immobilisation step, rubber gaskets with a 9 mm diameter circular opening were placed on all surfaces that were immobilised with DNA labelled with Alexa 647 at the 5′ end and primed with 1×NEBuffer 4 (50 mM Potassium Acetate, 20 mM Tris-acetate, 10 mM Magnesium Acetate, 1 mM DTT, pH 7.9) for 10 mins. Note that for sample D, the immobilised hairpin DNA does not contain inosine and inosine is replaced by guanine. All the samples were subsequently exposed to either NEBuffer 4 containing 1.5 U/μl Endonuclease V (sample A, B and D) or NEBuffer 4 without Endonuclease V (sample C) for 1 hr. All the samples were subsequently washed with 1×T3 DNA Ligase buffer (66 mM Tris-HCl, 10 mM MgCl₂, 1 mM ATP, 7.5% PEG6000, 1 mM DTT, pH 7.6), 1×T3 DNA Ligase buffer containing 1M sodium chloride and 0.05% (v/v) Tween20, washed again with 1×T3 DNA Ligase buffer and scanned on ChemiDoc Imager in the Alexa 647 channel.

### Results

FIG. 51 shows fluorescence signals from hairpin DNA oligomers before and after cleavage reactions.

FIG. 52 shows measured fluorescence signals before and after cleavage reactions obtained from DNA immobilised surfaces as described above. Successful cleavage reactions were only observed from samples A and B, while fluorescence signal intensities remained almost the same for samples C and D due to absence of either Endonuclease V (sample C) or inosine in the sequence (sample D).

Significant reductions in the fluorescence signals were observed from samples A and B as a result of successful cleavage reactions at the inosine site within the DNA strand with the presence of Endonuclease V. For samples C and D, absence of Endonuclease V and lack of inosine in the DNA respectively resulted in the fluorescence signals to remain almost the same level as the initial signals obtained after DNA immobilisation.

(2): Ligation Reactions

Materials and Methods

After the cleavage reaction as described in (1), samples A and B (as described in FIG. 50a) were exposed to 1×T3 DNA Ligase buffer containing MnCl₂ (2 mM), inosine strands labelled with Alexa 647 at the 5′ end (16 μM) and complimentary ‘helper’ strands (16 μM) (the sequences are shown in FIG. 53 below) with T3 DNA ligase (250 U/μl) for sample A, and without T3 DNA Ligase as a negative control for sample B. Samples were incubated in the respective solutions for 1 hr. After 1 hr, the surfaces were washed in water, subsequently exposed to water containing 1M sodium chloride and 0.05% (v/v) Tween20, and washed again with water. Fluorescence signals from the surfaces were measured using ChemiDoc in the Alexa 647 channels. FIG. 53 shows the sequences for the inosine-containing strand and the complimentary ‘helper’ strand for ligation reactions.

### Results

FIG. 54 shows results relating to the monitoring of ligation reactions. Fluorescence signals detected from Alexa 647 channel before and after ligation reactions. An increase in fluorescence signals in the Alexa 647 channels after ligation were only obtained from sample A with T3 DNA ligase, while fluorescence signals remained at the same level after ligation reaction for sample B due to the absence of T3 DNA ligase.

FIG. 55 shows that a significant increase in the Alexa 647 fluorescence signal was obtained after ligation reaction from sample A as a result of the successful ligation, where the signal level recovers to the initial signal level after DNA immobilisation and prior to cleavage reaction as shown in FIG. 52. The fluorescence signals from the sample B remained the same after the ligation reaction due to the absence of T3 DNA ligase.

### Conclusion

The results in this Example demonstrate that a molecule comprising a support strand and a synthesis strand for use in the methods of the present invention, can readily be immobilised on a surface substrate compatible with the polynucleotide synthesis reactions described herein and can be subjected to cleavage and ligation reactions whilst at the same time remaining stable and attached to the substrate.

### Example 13. Incorporation of 3′-O-Azidomethyl-dNTPs to the 3′ Terminal End of Blunt-Ended DNA

This example describes the incorporation of 3′-O-azidomethyl-dNTPs to the 3′ end of blunt-ended double-stranded DNA.

The steps below demonstrate the controlled addition of a 3′-O-protected single nucleotide to a blunt-ended double-stranded oligonucleotide by enzymatic incorporation by DNA polymerase. The steps are in accordance with incorporation step 4 as shown in each of FIGS. 1 to 10.

### Materials and Methods

### Materials

1. In-house synthesised 3′-O-azidomethyl-dNTPs.
2. Therminator X DNA polymerase that has been engineered by New England Biolabs to possess enhanced ability to incorporate 3-O-modified dNTPs.
3. Blunt-ended double-stranded DNA oligonucleotide.
   Four Types of Reversible Terminators were Tested:

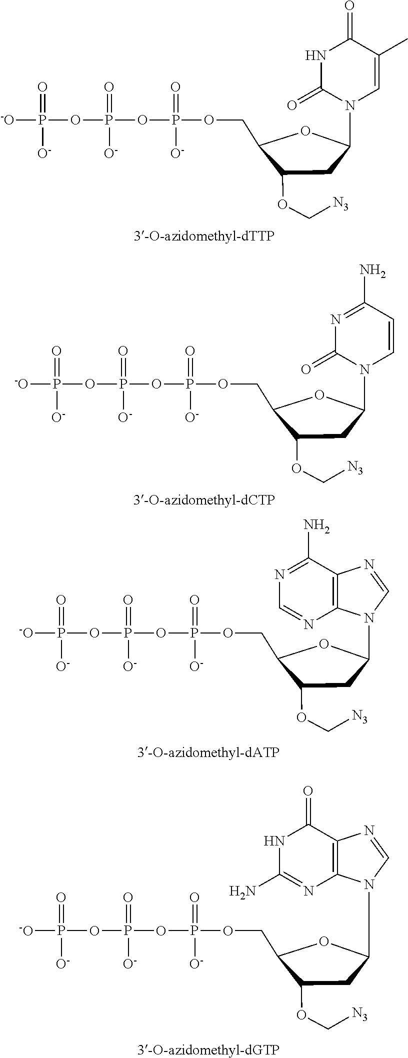

### Method

1. 5 μl of 10× Thermopol® buffer (20 mM Tris-HCl, 10 mM (NH₄)₂SO₄, 10 mM KCl, 2 mM MgSO₄, 0.1% Triton® X-100, pH 8.8, New England Biolabs) was mixed with 33.5 μl of sterile deionized water (ELGA VEOLIA) in a 1.5 ml Eppendorf tube.
2. 2 μl of 20 μM primer (40 pmol, 1 equiv) (SEQ ID: NO: 68, FIG. 56a) and 3 μl of 20 μM template (60 pmol, 1.5 equiv) (SEQ ID: NO: 69, FIG. 56a) were added to the reaction mixture.
3. 3′-O-modified-dTTP (2 μl of 100 μM) and MnCl₂ (2.5 μl of 40 mM) were added.
4. 2 μl of Therminator X DNA polymerase (20 U, New England BioLabs) was then added.
5. The reaction was incubated for 30 minutes at 37° C.
6. The reaction was stopped by addition of TBE-Urea sample buffer (Novex).
7. The reaction was separated on polyacrylamide gel (15%) TBE buffer and visualized by ChemiDoc MP imaging system (BioRad).

### Results

FIG. 56b depicts a gel showing results of incorporation of 3′-O-modified-dNTPs by Therminator X DNA polymerase in the presence of Mn2+ ions at 37° C. The data show that Therminator X DNA polymerase was successfully able to incorporate 3′-O-modified-dNTPs to the 3′ terminal end of the blunt ended DNA oligonucleotide to create a single base overhang.

### Example 14. Exemplary Ligation of a Polynucleotide Ligation Molecule to a Scaffold Polynucleotide Using Blunt End Ligation

This example describes the ligation of a polynucleotide ligation molecule, using DNA ligase, to a scaffold polynucleotide. This example involves ligation of molecules with blunt ends, consistent with synthesis method of the invention version 2, as depicted in FIG. 2.

FIG. 57 provides a scheme depicting a DNA synthesis reaction cycle. The scheme is intended to be consistent with synthesis method of the invention version 2 as depicted in FIG. 2. Thus the scheme in FIG. 57 shows the provision of a scaffold polynucleotide (right hand hairpin structure in the uppermost panel of the scheme) having a blunt end, the left strand corresponds to the support strand and the right strand corresponds to the synthesis strand. The terminal nucleotide of the support strand comprises a phosphate group at the 5′ end. In the next step of the cycle a polynucleotide ligation molecule (far right structure in the uppermost panel of the scheme) is provided. The polynucleotide ligation molecule has a support strand (the left strand) and a helper strand (the right strand). The polynucleotide ligation molecule has a complementary ligation end which is a blunt end and which comprises a universal nucleotide which is 2-deoxyinosine (In). The terminal end of the helper strand at the complementary ligation end comprises a non-ligatable nucleotide. The terminal end of the support strand at the complementary ligation end comprises a nucleotide of the predefined sequence, depicted as “A”, purely for illustration. Upon ligation of the polynucleotide ligation molecule to the scaffold polynucleotide, the terminal nucleotide of the support strand at the complementary ligation end of the polynucleotide ligation molecule is ligated to the terminal nucleotide of the support strand of the scaffold polynucleotide, and a single strand break (“nick”) is created between the helper strand of the polynucleotide ligation molecule and the synthesis strand of the scaffold polynucleotide. The terminal nucleotide of the support strand of the polynucleotide ligation molecule comprises a nucleotide of the predefined sequence and occupies position n. The universal nucleotide consequently occupies position n+2. Following ligation the polynucleotide ligation molecule is cleaved by cleaving the support strand between positions n and n+1. In the reaction scheme shown in FIG. 57 the helper strand is shown removed prior to cleavage, as an optional step. Following cleavage the nucleotide of the predefined sequence is retained in the scaffold polynucleotide. In the next step a further nucleotide, depicted as “T”, purely for illustration, is incorporated in this case into the synthesis strand of the scaffold polynucleotide by the action of a polymerase enzyme or a nucleotide transferase enzyme. The further nucleotide comprises a reversible terminator group or blocking group. The further nucleotide pairs with the terminal nucleotide of the support strand to form a nucleotide pair. The scheme then depicts a deprotection or deblocking step wherein the reversible terminator group or blocking group is removed, thus completing a cycle of synthesis.

As detailed below, this Example 14 describes the step of ligating the polynucleotide ligation molecule to the scaffold polynucleotide as shown in the dashed box in FIG. 57.

### Materials and Methods

### Materials:

1. Oligonucleotides utilized in this example were designed in house and synthesised by Integrated DNA technologies. These are described in FIG. 58.
2. The oligonucleotides were diluted to a stock concentration of 100 uM using sterile distilled water (ELGA VEOLIA).

### Method:

Ligation reactions with oligonucleotides were carried out using the procedure below:
1. 12 μl of sterile distilled water (ELGA VEOLIA) was added into a 1.5 ml Eppendorf tube.
2. 30 μl of 2×T3 DNA Ligase Reaction Buffer NEB (132 mM Tris-HCl, 20 mM MgCl₂, 2 mM Dithiothreitol, 2 mM ATP, 15% Polyethylene glycol (PEG6000) and pH 7.6 at 25° C.) and 2 μl of 40 mM MnCl₂ were then added into the same Eppendorf tube.
3. 5 μl of 2-deoxyinosine (In) strand (200 mol/l) (SEQ ID: No 71) and 5 μl of helper strand (200 mol/l) (SEQ ID: No 72) and 1 μl TAMRA or any fluorescently tagged blunt ended polynucleotide (20 mol/l) (SEQ ID: No 70) were added into the same tube.
4. 5 μl of T3 DNA Ligase NEB (3000 units/0) were added into the same tube.
5. The reaction mixture was then incubated at room temperature for 30 minutes.
6. After the incubation time had elapsed, the reaction was terminated with the addition of TBE-Urea sample Buffer (Novex).

### Results

The results are shown in FIG. 59.

### Example 15. Exemplary Ligation of a Polynucleotide Ligation Molecule to a Scaffold Polynucleotide Using Overhanging End Ligation

This example describes the ligation of a polynucleotide ligation molecule, using DNA ligase, to a scaffold polynucleotide. This example involves ligation of molecules with overhanging ends, consistent with synthesis method of the invention version 4 as depicted in FIG. 5, and further variants of synthesis method of the invention version 4 as depicted in FIG. 8.

FIG. 60 provides a scheme depicting a DNA synthesis reaction cycle. The scheme is intended to be consistent with synthesis method of the invention version 4 as depicted in FIG. 5. Thus the scheme in FIG. 60 shows the provision of a scaffold polynucleotide (right hand hairpin structure in the uppermost panel of the scheme) having an overhanging end, the left strand corresponds to the support strand and the right strand corresponds to the synthesis strand. The terminal nucleotide of the support strand occupies position n and overhangs the terminal nucleotide of the synthesis strand. The terminal nucleotide of the support strand is depicted as “T” for illustrative purposes only and comprises a phosphate group at the 5′ end. In the next step of the cycle a polynucleotide ligation molecule (far right structure in the uppermost panel of the scheme) is provided. The polynucleotide ligation molecule has a support strand (the left strand) and a helper strand (the right strand). The polynucleotide ligation molecule has a complementary ligation end having an overhanging end and which comprises a universal nucleotide which is 2-deoxyinosine (In). The terminal end of the helper strand at the complementary ligation end comprises a non-ligatable nucleotide and overhangs the terminal nucleotide of the support strand. The terminal end of the support strand at the complementary ligation end comprises a nucleotide of the predefined sequence, depicted as “G”, purely for illustration. Upon ligation of the polynucleotide ligation molecule to the scaffold polynucleotide, the terminal nucleotide of the support strand at the complementary ligation end of the polynucleotide ligation molecule is ligated to the terminal nucleotide of the support strand of the scaffold polynucleotide, and a single strand break (“nick”) is created between the helper strand of the polynucleotide ligation molecule and the synthesis strand of the scaffold polynucleotide. The terminal nucleotide of the support strand of the polynucleotide ligation molecule comprises a nucleotide of the predefined sequence and occupies position n+1. The universal nucleotide consequently occupies position n+3.

Following ligation the polynucleotide ligation molecule is cleaved by cleaving the support strand between positions n+1 and n+2. In the reaction scheme shown in FIG. 60 the helper strand is shown removed prior to cleavage, as an optional step. Following cleavage the nucleotide of the predefined sequence is retained in the scaffold polynucleotide as the terminal nucleotide of the support strand in an overhanging end. In the next step a further nucleotide, depicted as “A”, purely for illustration, is incorporated in this case into the synthesis strand of the scaffold polynucleotide by the action of a polymerase enzyme or a nucleotide transferase enzyme. The further nucleotide comprises a reversible terminator group or blocking group. The further nucleotide pairs with the partner nucleotide in the support strand, in this case depicted as “T”, purely for illustration, thus forming a nucleotide pair. The scheme then depicts a deprotection or deblocking step wherein the reversible terminator group or blocking group is removed, thus completing a cycle of synthesis.

FIG. 61 provides a similar scheme depicting a DNA synthesis reaction cycle. The scheme is intended to be consistent with further variants of synthesis method of the invention version 4 as depicted in FIG. 8, wherein the length of the overhang can be extended from a single base overhang to a two, three, four or more base overhang.

As detailed below, this Example 15 describes the step of ligating the polynucleotide ligation molecule to the scaffold polynucleotide as shown in the dashed box in FIGS. 60 and 61.

### Materials and Methods

### Materials:

1. Oligonucleotides utilized in this example were designed in house and synthesised by Integrated DNA technologies. These are described in FIG. 62.
2. The oligonucleotides were diluted to a stock concentration of 100 uM using sterile distilled water (ELGA VEOLIA).

### Method.

Ligation reactions with oligonucleotides were carried out using the procedure below:
1. 12 μl of sterile distilled water (ELGA VEOLIA) was added into a 1.5 ml Eppendorf tube.
2. 30 μl of 2×T3 DNA Ligase Reaction buffer NEB (132 mM Tris-HCl, 20 mM MgCl₂, 2 mM Dithiothreitol, 2 mM ATP, 15% Polyethylene glycol (PEG6000) and pH 7.6 at 25° C.) and 2 μl of 40 mM MnCl₂ were then added into the same Eppendorf tube.
3. 5 μl of 2-deoxyinosine (In) strand (200 μmol/l) (SEQ ID: No 78) and 5 μl of helper strand (200 μmol/l) (SEQ ID: No 79, 80) and 1 μl TAMRA or any fluorescently tagged polynucleotide with a 5′ overhang (20 μmol/l) (SEQ ID: No 73, 74, 75, 76 or 77) were added into the same tube.
4. 5 μl of T3 DNA Ligase NEB (3000 units/μ1) were added into the same tube.
5. The reaction mixture was then incubated at room temperature for 30 minutes.
6. After the incubation time had elapsed, the reaction was terminated with the addition of TBE-Urea sample Buffer (Novex).

### Results

The results are shown in FIG. 63.

### Example 16. Exemplary Ligation of a Polynucleotide Ligation Molecule to the 3′End of a Blunt-Ended Hairpin Polynucleotide Followed by Site-Specific Cleavage

This example describes the addition of a single nucleotide (guanosine) to the 3′ end of a blunt-ended hairpin polynucleotide by ligation of a polynucleotide ligation molecule comprising 2-deoxyuridine, used as a universal nucleotide, followed by site-specific cleavage of the 1^{st} phosphodiester bond 5′ to the uridine site as depicted in FIG. 64A.

### Ligation Step

The ligation step describes the protocol for the ligation of a polynucleotide ligation molecule to the 3′ end of a blunt-ended polynucleotide using DNA ligase in the presence of a helper strand. The polynucleotide ligation molecule comprising a universal nucleotide (uridine) requires phosphorylation at the 5′ end. In order to prevent ligation of the helper strand, the 3′-end of the helper strand is blocked by the presence of a 2′,3′-dideoxynucleotide at the 3′ end, which is complementary to the terminal nucleotide at the 5′ end of the polynucleotide ligation molecule comprising the universal nucleotide (uridine).

### Materials and Methods

### Materials

1. Oligonucleotides utilized in this example were designed in house and synthesised by Integrated DNA technologies. These are described in FIG. 64C.
2. The oligonucleotides were diluted to a stock concentration of 100 μM using sterile distilled water (ELGA VEOLIA).

### Method

Ligation reactions with oligonucleotides were carried out using the procedure below:
1. 30 μl of 2×T3 DNA Ligase Reaction buffer NEB (132 mM Tris-HCl, 20 mM MgCl₂, 2 mM Dithiothreitol, 2 mM ATP, 15% Polyethylene glycol (PEG6000) and pH 7.6 at 25° C.) and 2 μl of 40 mM MnCl₂ were added into the same Eppendorf tube.
2. 2.8 μl of sterile distilled water (ELGA VEOLIA) was added into a 1.5 ml Eppendorf tube.
3. 10 μl of 2-deoxyuridine (U) strand (200 mol/l) (SEQ ID: No 85) and 10 μl of helper strand (200 mol/l) (SEQ ID: No 86) were added to the same tube.
4. 0.2 μl TAMRA or any fluorescently tagged blunt-ended hairpin polynucleotide (SEQ ID: No 84) (10 mol/l) was added into the same tube.
5. 5 μl of T3 DNA Ligase NEB (3000 units/0) was added into the same tube.
6. The reaction mixture was then incubated at room temperature for 15 minutes.
7. After the incubation time had elapsed, the reaction was terminated with the addition of TBE-Urea sample Buffer (Novex) and a 5 μl aliquot was obtained for gel analysis.
8. After the incubation time had elapsed, the reaction mixture was purified using a QIAGEN nucleotide removal kit.

### Cleavage Step

The second step describes the cleavage of the ligated polynucleotide using both Uracil DNA glycosylase and AP endonuclease 1, which cleaves the 1^{st} phosphodiester bond 5′ to the universal nucleotide (uracil).

### Materials and Methods

### Materials:

1. Oligonucleotides utilized in this example were designed in house and synthesised by Integrated DNA technologies. These are described in FIG. 64C.
2. The oligonucleotides were diluted to a stock concentration of 100 μM using sterile deionised water (ELGA VEOLIA).

### Method:

Cleavage reactions with oligonucleotides were carried out using the procedure below:
1. 31 μl TAMRA or any fluorescently tagged polynucleotide from the ligation reaction was added into a 1.5 ml Eppendorf tube.
2. 4 μl of 10×NEB buffer 4 (500 mM Potassium Acetate, 200 mM Tris-acetate, 100 mM Magnesium Acetate, 1000 μg/ml bovine, pH 7.9) was then added into the same Eppendorf tube.
3. 1 μl of Uracil DNA Glycosylase (UDG) (10 unit/μl) and 5 μl AP Endonuclease I (NEB) (10 unit/μl) were added.
4. The reaction mixture was then gently mixed by resuspension with a pipette, centrifuged at 13,000 rpm for 5 seconds and incubated at room temperature for 30 minutes.
5. After the incubation time had elapsed, the reaction was terminated with the addition of Gel Loading Buffer II (Invitrogen).

### Results

The results are shown in FIG. 64.

### Example 17. Exemplary Ligation of a Polynucleotide Ligation Molecule to the 5′-End of a Hairpin Polynucleotide with a 3′ Single Base Overhang Followed by Site-Specific Cleavage

This example describes the addition of a single nucleotide (cytidine) to the 5′ end of a hairpin polynucleotide with a 3′ single base overhang by ligation of a polynucleotide ligation molecule comprising inosine, used as a universal nucleotide, followed by site-specific cleavage of the 2^{nd} phosphodiester bond 3′ to the inosine site as depicted in FIG. 65A.

### Ligation Step

The ligation step describes the protocol for the ligation of a polynucleotide ligation molecule to the 5′ end of 3′ single base overhung polynucleotide using DNA ligase in the presence of a helper strand. The helper strand lacks a phosphate at the 5′ end.

### Materials and Methods

### Materials:

1. Oligonucleotides utilized in this example were designed in house and synthesised by Integrated DNA technologies. These are described in FIG. 65C.
2. The oligonucleotides were diluted to a stock concentration of 100 μM using sterile distilled water (ELGA VEOLIA).

### Method:

Ligation reactions with oligonucleotides were carried out using the procedure below:
1. 30 μl of 2×T3 DNA Ligase Reaction buffer NEB (132 mM Tris-HCl, 20 mM MgCl₂, 2 mM Dithiothreitol, 2 mM ATP, 15% Polyethylene glycol (PEG6000) and pH 7.6 at 25° C.) and 2 μl of 40 mM MnCl₂ were added into the same Eppendorf tube.
2. 2.8 μl of sterile distilled water (ELGA VEOLIA) was added into a 1.5 ml Eppendorf tube.
3. 10 μl of 2-deoxyinosine (In) strand (200 mol/l) (SEQ ID: No 88) and 10 μl of helper strand (200 mol/l) (SEQ ID: No 89) were added to the same tube.
4. 0.2 μl TAMRA or any fluorescently tagged hairpin polynucleotide for ligation with 3′ single base overhang (SEQ ID: No 87) (10 mol/l) was added into the same tube.
5. 5 μl of T3 DNA Ligase NEB (3000 units/0) was added into the same tube.
6. The reaction mixture was then incubated at room temperature for 15 minutes.
7. After the incubation time had elapsed, the reaction was terminated by the addition of TBE-Urea sample Buffer (Novex) and a 5 μl aliquot was obtained for gel analysis.
8. After the incubation time had elapsed, the reaction mixture was purified using a QIAGEN nucleotide removal kit.

### Cleavage Step

The cleavage step describes the cleavage of the ligated polynucleotide using Endonuclease V, which cleaves the 2^{nd} phosphodiester bond 3′ to the universal nucleotide (inosine).

### Materials and Methods

### Materials:

1. Oligonucleotides utilized in this example were designed in house and synthesised by Integrated DNA technologies. These are described in FIG. 65C.
2. The oligonucleotides were diluted to a stock concentration of 100 μM using sterile deionised water (ELGA VEOLIA).

### Method.

Cleavage reaction with oligonucleotides were carried out using the procedure below:
1. 31 μl TAMRA or any fluorescently tagged polynucleotide from the ligation reaction was added into a 1.5 ml Eppendorf tube.
2. 4 μl of 10×NEB buffer 4 (500 mM Potassium Acetate, 200 mM Tris-acetate, 100 mM Magnesium Acetate, 1000 μg/ml bovine, pH 7.9) was then added into the same Eppendorf tube.
3. 5 μl of Endonuclease V (3 units/0) was added.
4. The reaction mixture was then gently mixed by resuspension with a pipette, centrifuged at 13,000 rpm for 5 seconds and incubated at room temperature for 30 minutes.
5. After the incubation time had elapsed, the reaction was terminated by the addition of TBE-Urea sample Buffer (Novex).

### Results

The results are shown in FIG. 65.

### Example 18. Exemplary Ligation of a Polynucleotide Ligation Molecule to the 5′-End of a Blunt-Ended Hairpin Polynucleotide Followed by Site-Specific Cleavage

This example describes the addition of single nucleotide (cytidine) to the 5′ end of a blunt-ended hairpin polynucleotide by ligation of a polynucleotide ligation molecule comprising uridine, used as a universal nucleotide, followed by site-specific cleavage of both 1st phosphodiester bond 3′ and 5′ to the uridine as depicted in FIG. 66A.

### Ligation Step

The ligation step describes the protocol for the ligation of a polynucleotide ligation molecule to the 5′ end of a blunt-ended polynucleotide using DNA ligase in the presence of a helper strand. The helper strand lacks a phosphate at the 5′ end.

### Materials and Methods

### Materials:

1. Oligonucleotides utilized in this example were designed in house and synthesised by Integrated DNA technologies. These are described in FIG. 66C.
2. The oligonucleotides were diluted to a stock concentration of 100 μM using sterile distilled water (ELGA VEOLIA).

### Method:

Ligation reactions with oligonucleotides were carried out using the procedure below:
1. 30 μl of 2×T3 DNA Ligase Reaction buffer NEB (132 mM Tris-HCl, 20 mM MgCl₂, 2 mM Dithiothreitol, 2 mM ATP, 15% Polyethylene glycol (PEG6000) and pH 7.6 at 25° C.) and 2 μl of 40 mM MnCl₂ were added into the same Eppendorf tube.
2. 2.8 μl of sterile distilled water (ELGA VEOLIA) was added into a 1.5 ml Eppendorf tube.
3. 10 μl of 2-deoxyuridine (U) strand (200 mol/l) (SEQ ID: No. 91) and 10 μl of helper strand (200 mol/l) (SEQ ID: No. 92) were added to the same tube.
4. 0.2 μl TAMRA or any fluorescently tagged blunt-ended hairpin polynucleotide (SEQ ID: No. 90) (10 mol/l) was added into the same tube.
5. 5 μl of T3 DNA Ligase NEB (3000 units/0) was added into the same tube.
6. The reaction mixture was then incubated at room temperature for 15 minutes.
7. After the incubation time had elapsed, the reaction was terminated by the addition of TBE-Urea sample Buffer (Novex) and a 5 μl aliquot was obtained for gel analysis.
8. After the incubation time had elapsed, the reaction mixture was purified using a QIAGEN nucleotide removal kit.

### Cleavage Step

The cleavage step describes the cleavage of the ligated polynucleotide using a reaction mixture containing both Uracil DNA glycosylase and Endonuclease VIII, which cleaves the 1^{st} phosphodiester bond 3′ and 5′ to the universal nucleotide (uridine).

### Materials and Methods

### Materials:

1. Oligonucleotides utilized in this example were designed in house and synthesised by Integrated DNA technologies. These are described in FIG. 66B.
2. The oligonucleotides were diluted to a stock concentration of 100 μM using sterile deionised water (ELGA VEOLIA).

### Method:

Cleavage reactions with oligonucleotides were carried out using the procedure below:
1. 31 μl TAMRA or any fluorescently tagged polynucleotide from the ligation reaction was added into a 1.5 ml Eppendorf tube.
2. 4 μl of 10× Cut Smart buffer (500 mM Potassium Acetate, 200 mM Tris-acetate, 100 mM Magnesium Acetate, 1000 μg/ml bovine, pH 7.9) was then added into the same Eppendorf tube.
3. 5 μl of USER enzyme (mixture of Uracil DNA Glycosylase (UDG) and Endonuclease VIII) (1 unit/μl) was added into the same tube.
4. The reaction mixture was then gently mixed by resuspension with a pipette, centrifuged at 13,000 rpm for 5 seconds and incubated at room temperature for 30 minutes.
5. After the incubation time had elapsed, the reaction was terminated by the addition of TBE-Urea sample Buffer (Novex).

### Results

The results are shown in FIG. 66.

### Example 19. Exemplary Ligation of a Polynucleotide Ligation Molecule to the 3′-End of Hairpin Polynucleotide with a 5′ Single Base Overhang Followed by Site-Specific Cleavage

This example describes the addition of a single nucleotide (guanosine) to the 3′ end of a hairpin polynucleotide with a 5′ single base overhang by ligation of a polynucleotide ligation molecule comprising uridine, used as a universal nucleotide, followed by site-specific cleavage of the 1^{st} phosphodiester bond 5′ to the uridine site as depicted in FIG. 67A.

### Ligation Step

The ligation step describes the protocol for the ligation of a polynucleotide to the 3′ end of a 5′ single base overhung polynucleotide using DNA ligase in the presence of a helper strand. The polynucleotide ligation molecule comprising a universal nucleotide (uridine) requires phosphorylation at the 5′ end. In order to prevent ligation of the helper strand, the 3′-end of the helper strand is blocked by the presence of a 2′,3′-dideoxynucleotide at the 3′ end, which is complementary to the universal nucleotide at the 5′ end of the polynucleotide ligation molecule.

### Materials and Methods

### Materials:

1. Oligonucleotides utilized in this example were designed in house and synthesised by Integrated DNA technologies. These are described in FIG. 67C.
2. The oligonucleotides were diluted to a stock concentration of 100 μM using sterile distilled water (ELGA VEOLIA).

### Method:

Ligation reactions with oligonucleotides were carried out using the procedure below:
1. 30 μl of 2×T3 DNA Ligase Reaction buffer NEB (132 mM Tris-HCl, 20 mM MgCl₂, 2 mM Dithiothreitol, 2 mM ATP, 15% Polyethylene glycol (PEG6000) and pH 7.6 at 25° C.) and 2 μl of 40 mM MnCl₂ were added into the same Eppendorf tube.
2. 2.8 μl of sterile distilled water (ELGA VEOLIA) was added into a 1.5 ml Eppendorf tube.
3. 10 μl of 2-deoxyuridine (U) strand (200 mol/l) (SEQ ID: No. 94) and 10 μl of helper strand (200 mol/l) (SEQ ID: No. 93) were added to the same tube.
4. 0.2 μl TAMRA or any fluorescently tagged hairpin polynucleotide with a 5′ single base overhang (SEQ ID: No. 93) (10 mol/l) was added into the same tube.
5. 5 μl of T3 DNA Ligase NEB (3000 units/0) was added into the same tube.
6. The reaction mixture was then incubated at room temperature for 15 minutes.
7. After the incubation time had elapsed, the reaction was terminated with the addition of TBE-Urea sample Buffer (Novex) and a 5 μl aliquot was obtained for gel analysis.
8. After the incubation time had elapsed, the reaction mixture was purified using a QIAGEN nucleotide removal kit.

### Cleavage Step

The cleavage step describes the cleavage of the ligated polynucleotide using a cleavage reaction mixture containing both Uracil DNA glycosylase and AP Endonuclease 1, which cleaves the 1^{st} phosphodiester bond 5′ to the universal nucleotide (uridine).

### Materials and Methods

### Materials:

1. Oligonucleotides utilized in this example were designed in house and synthesised by Integrated DNA technologies. These are described in FIG. 67C.
2. The oligonucleotides were diluted to a stock concentration of 100 μM using sterile deionised water (ELGA VEOLIA).

### Method:

Cleavage reactions with oligonucleotides were carried out using the procedure below:
1. 30 μl TAMRA or any fluorescently tagged polynucleotide from the ligation reaction was added into a 1.5 ml Eppendorf tube.
2. 4 μl of 10×NEB buffer 4 (500 mM Potassium Acetate, 200 mM Tris-acetate, 100 mM Magnesium Acetate, 10 mM DTT, pH 7.9) was then added into the same Eppendorf tube.
3. 1 μl of Uracil DNA Glycosylase (UDG) (5 U/μl) and 5 μl of AP Endonuclease I (NEB) (10 unit/μl) were added into the same tube.
4. The reaction mixture was then gently mixed by resuspension with a pipette, centrifuged at 13,000 rpm for 5 seconds and incubated at room temperature for 30 minutes.
5. After the incubation time had elapsed, the reaction was terminated by the addition of TBE-Urea sample Buffer (Novex).

### Results

The results are shown in FIG. 67.

### Example 20. Exemplary Ligation of a Polynucleotide Ligation Molecule to the 3′-End of Hairpin Polynucleotide with a 5′ Single Base Overhang Followed by Site-Specific Cleavage Leaving a Phosphate at the 3′ End

This example describes the addition of a single nucleotide (guanosine) to the 3′ end of a hairpin polynucleotide with a 5′ single base overhang by ligation of a polynucleotide ligation molecule comprising uridine, used as a universal nucleotide, followed by site-specific cleavage of both 1^{st} phosphodiester bonds 5′ and 3′ to the uridine leaving a phosphate attached to the 3′ end of the hairpin polynucleotide as depicted in FIG. 68.

### Ligation Step

The ligation step describes the protocol for the ligation of a polynucleotide ligation molecule to the 3′ end of a 5′ single base overhung polynucleotide using DNA ligase in the presence of a helper strand. The polynucleotide ligation molecule comprising the universal nucleotide (uridine) requires phosphorylation at 5′ end. In order to prevent ligation of the helper strand, the 3′-end of the helper strand is blocked by the presence of a 2′,3′-dideoxynucleotide at the 3′ end, which is complementary to the universal nucleotide at the 5′ end of the polynucleotide ligation molecule.

### Materials and Methods

### Materials:

1. Oligonucleotides utilized in this example were designed in house and synthesised by Integrated DNA technologies. These are described in FIG. 68C.
2. The oligonucleotides were diluted to a stock concentration of 100 μM using sterile distilled water (ELGA VEOLIA).

### Method:

Ligation reactions with oligonucleotides were carried out using the procedure below:
1. 30 μl of 2×T3 DNA Ligase Reaction buffer NEB (132 mM Tris-HCl, 20 mM MgCl₂, 2 mM Dithiothreitol, 2 mM ATP, 15% Polyethylene glycol (PEG6000) and pH 7.6 at 25° C.) and 2 μl of 40 mM MnCl₂ were added into the same Eppendorf tube.
2. 2.8 μl of sterile distilled water (ELGA VEOLIA) was added into a 1.5 ml Eppendorf tube.
3. 10 μl of 2-deoxyuridine (U) strand (200 mol/l) (SEQ ID: No. 97) and 10 μl of helper strand (200 mol/l) (SEQ ID: No. 98) were added to the same tube.
4. 0.2 μl TAMRA or any fluorescently tagged hairpin polynucleotide containing 2′-deoxyuridine (SEQ ID: No. 96) (10 mol/l) was added into the same tube.
5. 5 μl of T3 DNA Ligase NEB (3000 units/0) was added into the same tube.
6. The reaction mixture was then incubated at room temperature for 15 minutes.
7. After the incubation time had elapsed, the reaction was terminated with the addition of TBE-Urea sample Buffer (Novex) and a 5 μl aliquot was obtained for gel analysis.
8. After the incubation time had elapsed, the reaction mixture was purified using a QIAGEN nucleotide removal kit.

### Cleavage Step

The cleavage step describes the cleavage of polynucleotides using a cleavage reaction mixture containing both Uracil DNA glycosylase and Endonuclease VIII, which cleaves both of the 1^{st} phosphodiester bonds 5′ and 3′ to the universal nucleotide (uridine), leaving a 3′ phosphate on the hairpin polynucleotide.

### Materials and Methods

### Materials:

1. Oligonucleotides utilized in this example were designed in house and synthesised by Integrated DNA technologies. These are described in FIG. 68C.
2. The oligonucleotides were diluted to a stock concentration of 100 μM using sterile deionised water (ELGA VEOLIA).

### Method:

Cleavage reactions with oligonucleotides were carried out using the procedure below:
1. 31 μl TAMRA or any fluorescently tagged polynucleotide from the ligation reaction was added into a 1.5 ml Eppendorf tube.
2. 4 μl of 10× Cut Smart buffer (500 mM Potassium Acetate, 200 mM Tris-acetate, 100 mM Magnesium Acetate, 1000 μg/ml bovine, pH 7.9) was then added into the same Eppendorf tube.
3. 5 μl of USER enzyme (mixture of Uracil DNA Glycosylase (UDG) and Endonuclease VIII (NEB) (1 unit/μl) was added into the same tube.
4. The reaction mixture was then gently mixed by resuspension with a pipette, centrifuged at 13,000 rpm for 5 seconds and incubated at room temperature for 30 minutes.
5. After the incubation time had elapsed, the reaction was terminated by enzymatic heat inactivation (i.e. 95° C. for 10 mins).

### Results

The results are shown in FIG. 68.

### Example 21. Exemplary Removal of a Phosphate Group at the 3′ End of a Cleaved Hairpin Followed by Ligation of a Polynucleotide Ligation Molecule to the 3′-End of a Blunt-Ended Hairpin

This example describes the removal of a phosphate group at the 3′ end of a blunt-ended hairpin polynucleotide using Endonuclease IV followed by ligation of a polynucleotide ligation molecule to the 3′ end of a blunt-ended hairpin polynucleotide. Ligation of a polynucleotide ligation molecule to a 3′-phosphorylated blunt-ended polynucleotide was performed as a negative control.

### Dephosphorylation Step

The dephosphorylation step removes the phosphate group at the 3′ end leaving the phosphate group at the 5′ end intact, and it is performed with Endonuclease IV.

### Materials and Methods

### Materials:

1. Oligonucleotides used in this example were designed in house and synthesised by Integrated DNA technologies. These are described in FIG. 69C.
2. The oligonucleotides were diluted to a stock concentration of 100 μM using sterile deionised water (ELGA VEOLIA).

### Method:

The dephosphorylation reaction with oligonucleotides was carried out using the procedure below:
1. 48 μl deionised water was added into a 1.5 ml Eppendorf Tube.
2. 1 μl TAMRA or any fluorescently tagged polynucleotide (10 μM) was then added into the same Eppendorf tube.
3. 6 μl of 10×NEB buffer 3 (1000 mM NaCl, 500 mM Tris-HCl, 100 mM Magnesium chloride, 10 mM DTT, pH 7.9) was then added into the same Eppendorf tube.
4. 5 μl of Endonuclease IV (1 unit/μl) was added into the same tube.
5. The reaction mixture was then gently mixed by resuspension with a pipette, centrifuged at 13,000 rpm for 5 seconds and incubated at room temperature for 30 minutes.
6. After the incubation time had elapsed, the reaction was terminated by enzymatic heat inactivation (i.e. 95° C. for 10 mins).

### Ligation Step

The ligation step describes the ligation of the oligonucleotide to the 3′ end of a dephosphorylated blunt-ended polynucleotide using DNA ligase in the presence of a helper strand. The ligation of a non-dephosphorylated blunt-ended hairpin is performed as a negative control. The polynucleotide ligation molecule comprising a universal nucleotide (uridine) requires phosphorylation at the 5′ end. In order to prevent ligation of the helper strand, the 3′-end of the helper strand is blocked by the presence of a 2′,3′-dideoxynucleotide at 3′ end, which is complementary to the terminal nucleotide at the 5′ end of the polynucleotide ligation molecule comprising the universal nucleotide (uridine).

### Materials and Methods

### Materials:

1. Oligonucleotides used in this example were designed in house and synthesised by Integrated DNA technologies.
2. The oligonucleotides were diluted to a stock concentration of 100 uM using sterile distilled water (ELGA VEOLIA).

### Method:

Ligation reactions with oligonucleotides were carried out using the procedure below:
1. 30 μl of 2×T3 DNA Ligase Reaction buffer NEB (132 mM Tris-HCl, 20 mM MgCl₂, 2 mM Dithiothreitol, 2 mM ATP, 15% Polyethylene glycol (PEG6000) and pH 7.6 at 25° C.) and 2 μl of 40 mM MnCl₂ were added into the same Eppendorf tube.
2. 2.8 μl of sterile distilled water (ELGA VEOLIA) was added into a 1.5 ml Eppendorf tube.
3. 10 μl of 2-deoxyuridine (U) strand (200 mol/l) (SEQ ID: No 100) and 10 μl of helper strand (200 mol/l) (SEQ ID: No 101) were added to the same tube.
4. 0.2 μl TAMRA or any fluorescently tagged hairpin polynucleotide (SEQ ID: No 99) (10 mol/l) was added into the same tube.
5. 5 μl of T3 DNA Ligase NEB (3000 units/0) was added into the same tube.
6. The reaction mixture was then incubated at room temperature for 15 minutes.
7. After the incubation time had elapsed, the reaction was terminated with the addition of TBE-Urea sample Buffer (Novex) and a 5 μl aliquot was obtained for gel analysis.
8. After the incubation time had elapsed, the reaction mixture was purified using a QIAGEN nucleotide removal kit.

### Results

The results are shown in FIG. 68.

In the above Examples, all oligonucleotides presented in SEQ ID NOS 1-101 have a hydroxyl group at the 3′ terminus except for SEQ ID No 85, 86, 89, 92, 94, 95, 97, 98, 99, 100, 101. All oligonucleotides presented in SEQ ID NOS 1-101 lack a phosphate group at the 5′ terminus except for SEQ ID NO 7, SEQ ID NO 18, SEQ ID NO 35, SEQ ID NO 70 and SEQ ID NOS 73 to 77, as well as SEQ ID NOS 84, 85, 87, 90, 93, 94, 96, 97, 99, 100.

It is to be understood that different applications of the disclosed methods and products may be tailored to the specific needs in the art. It is also to be understood that the terminology used herein is for the purpose of describing particular embodiments of the invention only, and is not intended to be limiting.

As used in this specification and the appended claims, the singular forms “a”, “an”, and “the” include plural referents unless the content clearly dictates otherwise. Thus, for example, reference to “a polynucleotide ligation molecule” includes two or more such polynucleotides, reference to “a scaffold polynucleotide” includes two or more such scaffold polynucleotides, and the like.

All publications, patents and patent applications cited herein are hereby incorporated by reference in their entirety.

1. An in vitro method of synthesising a double-stranded polynucleotide wherein at least one strand has a predefined sequence, the method comprising performing cycles of synthesis wherein in each cycle one strand of a double-stranded polynucleotide is extended by the incorporation of one or more nucleotides in a first ligation reaction by the action of an enzyme having ligase activity, and the opposite strand of the double-stranded polynucleotide is extended by the incorporation of one or more nucleotides in a second ligation reaction by the action of an enzyme having ligase activity, wherein both strands are extended at the same terminal end of the double-stranded polynucleotide.

2. A method according to claim 1, wherein: (i) at least one strand has a predefined sequence, and wherein the nucleotides that are incorporated into said strand are nucleotides of the predefined sequence; or (ii) wherein both strands have a predefined sequence, and wherein the nucleotides that are incorporated into one strand are nucleotides of the predefined sequence of that strand, and wherein the nucleotides that are incorporated into the opposite strand are nucleotides of the predefined sequence of the opposite strand.

3. A method according to claim 2, wherein in a cycle of synthesis:
c) the 3′ end of one strand is extended by the incorporation of one or more nucleotides, and then
d) the 5′ end of the opposite strand is extended by the incorporation of one or more nucleotides.

4. A method according to claim 2, wherein in a cycle of synthesis:
c) the 5′ end of one strand is extended by the incorporation of one or more nucleotides, and then
d) the 3′ end of the opposite strand is extended by the incorporation of one or more nucleotides.

5. A method according to claim 3 or claim 4, wherein in a cycle of synthesis one strand is extended by the incorporation of a first nucleotide, and the opposite strand is extended by the incorporation of a second nucleotide which pairs with the first nucleotide.

6. A method according to claim 3 or claim 4, wherein in a cycle of synthesis one strand is extended by the incorporation of two nucleotides, and the opposite strand is extended by the incorporation of two nucleotides, thereby forming two nucleotide pairs.

7. A method according to any one of the preceding claims, wherein each cycle of synthesis comprises steps comprising:
(1) providing a double-stranded scaffold polynucleotide;
(2) extending a first strand of the scaffold polynucleotide by incorporating one or more nucleotides into the first strand;
(3) subjecting the first strand to a cleavage step, wherein the one or more nucleotides are retained in the first strand of the scaffold polynucleotide following cleavage;
(4) extending the second strand of the scaffold polynucleotide by incorporating one or more nucleotides into the second strand; and
(5) subjecting the second strand to a cleavage step, wherein the one or more nucleotides are retained in the second strand of the scaffold polynucleotide following cleavage.

8. A method according to claim 7, wherein the cleavage site in step (3) and in step (5) is defined by a polynucleotide sequence in the strand to be cleaved comprising a universal nucleotide.

9. A method according to claim 8, wherein in step (1) the double-stranded scaffold polynucleotide is provided with a ligation end and an opposite end; and wherein in steps (2) and (4) the one or more nucleotides of the predefined sequence are provided by first and second polynucleotide ligation molecules which are ligated to the ligation end of the scaffold polynucleotide by the action of the enzyme, wherein a polynucleotide ligation molecule comprises a universal nucleotide, and wherein upon ligation of a polynucleotide ligation molecule to the scaffold polynucleotide a strand of the scaffold polynucleotide is extended and a cleavage site defined by the universal nucleotide is created in the scaffold polynucleotide.

10. A method according to claim 9, wherein a polynucleotide ligation molecule is a double-stranded polynucleotide molecule comprising a synthesis strand and a helper strand hybridised thereto, and further comprising a complementary ligation end, the ligation end comprising:
(i) in the synthesis strand: (a) the one or more nucleotides for extending the scaffold polynucleotide positioned at the terminal end of the synthesis strand, and (b) the universal nucleotide; and
(ii) in the helper strand a non-ligatable terminal nucleotide.

11. A method according to claim 10, wherein:
(A) in step (1) the double-stranded scaffold polynucleotide is provided with a single base overhang, with the terminal nucleotide of the second strand overhanging the terminal nucleotide of the first strand;
(B) in step (2), in the first polynucleotide ligation molecule the terminal nucleotide of the synthesis strand occupies position n, wherein position n is the nucleotide position which is occupied by the first nucleotide to be added to the terminal end of the first strand of the scaffold polynucleotide in step (2); the penultimate nucleotide of the synthesis strand occupies position n+1, wherein position n+1 is the nucleotide position which is occupied by the second nucleotide to be added to the terminal end of the first strand of the scaffold polynucleotide in step (2); the universal nucleotide occupies position n+2 in the synthesis strand and is paired with the penultimate nucleotide of the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand;
(C) in step (3) the first strand of the ligated scaffold polynucleotide is cleaved between positions n+1 and n+2 whereupon the universal nucleotide is removed from the scaffold polynucleotide and the first and second nucleotides of the first polynucleotide ligation molecule are retained in the scaffold polynucleotide, and whereupon a single base overhang is created in the scaffold polynucleotide with the terminal nucleotide of the first strand overhanging the terminal nucleotide of the second strand;
(D) in step (4), in the second polynucleotide ligation molecule the terminal nucleotide of the synthesis strand occupies position n+1, wherein position n+1 is the nucleotide position which is occupied by the first nucleotide to be added to the terminal end of the second strand of the scaffold polynucleotide in step (4) and will be paired with the second nucleotide which was added to the terminal end of the first strand in step (2); the penultimate nucleotide of the synthesis strand occupies position n+2, wherein position n+2 is the nucleotide position which is occupied by the second nucleotide to be added to the terminal end of the second strand of the scaffold polynucleotide in step (4); the universal nucleotide occupies position n+3 in the synthesis strand and is paired with the penultimate nucleotide of the helper strand; the terminal nucleotide of the helper strand is a non-ligatable nucleotide;
and the complementary ligation end is provided with a single base overhang, with the terminal nucleotide of the synthesis strand overhanging the terminal nucleotide of the helper strand; and
(E) in step (5) the second strand of the ligated scaffold polynucleotide is cleaved between positions n+2 and n+3 whereupon the universal nucleotide is removed from the scaffold polynucleotide and the first and second nucleotides of the second polynucleotide ligation molecule are retained in the scaffold polynucleotide, and whereupon a single base overhang is created in the scaffold polynucleotide with the terminal nucleotide of the second strand overhanging the terminal nucleotide of the first strand.

12. A method according to claim 10, wherein:
(A) in step (1) the double-stranded scaffold polynucleotide is provided with a single base overhang, with the terminal nucleotide of the second strand overhanging the terminal nucleotide of the first strand;
(B) in step (2), in the first polynucleotide ligation molecule the terminal nucleotide of the synthesis strand occupies position n, wherein position n is the nucleotide position which is occupied by the first nucleotide to be added to the terminal end of the first strand of the scaffold polynucleotide in step (2); the penultimate nucleotide of the synthesis strand occupies position n+1, wherein position n+1 is the nucleotide position which is occupied by the second nucleotide to be added to the terminal end of the first strand of the scaffold polynucleotide in step (2); the universal nucleotide occupies position n+2 in the synthesis strand and is paired with the penultimate nucleotide of the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand;
(C) in step (3) the first strand of the ligated scaffold polynucleotide is cleaved between positions n+1 and n+2 whereupon the universal nucleotide is removed from the scaffold polynucleotide and the first and second nucleotides of the first polynucleotide ligation molecule are retained in the scaffold polynucleotide, and whereupon a single base overhang is created in the scaffold polynucleotide with the terminal nucleotide of the first strand overhanging the terminal nucleotide of the second strand;
(D) in step (4), in the second polynucleotide ligation molecule the terminal nucleotide of the synthesis strand occupies position n+1, wherein position n+1 is the nucleotide position which is occupied by the first nucleotide to be added to the terminal end of the second strand of the scaffold polynucleotide in step (4) and will be paired with the second nucleotide which was added to the terminal end of the first strand in step (2); the penultimate nucleotide of the synthesis strand occupies position n+2, wherein position n+2 is the nucleotide position which is occupied by the second nucleotide to be added to the terminal end of the second strand of the scaffold polynucleotide in step (4); the universal nucleotide occupies position n+4 in the synthesis strand and is paired with the nucleotide in the helper strand which is next to the penultimate nucleotide of the helper strand in the direction distal to the complementary ligation end; the terminal nucleotide of the helper strand is a non-ligatable nucleotide; and the complementary ligation end is provided with a single base overhang, with the terminal nucleotide of the synthesis strand overhanging the terminal nucleotide of the helper strand; and
(E) in step (5) the second strand of the ligated scaffold polynucleotide is cleaved between positions n+2 and n+3 whereupon the universal nucleotide is removed from the scaffold polynucleotide and the first and second nucleotides of the second polynucleotide ligation molecule are retained in the scaffold polynucleotide, and whereupon a single base overhang is created in the scaffold polynucleotide with the terminal nucleotide of the second strand overhanging the terminal nucleotide of the first strand.

13. A method according to claim 12, wherein:
(i) in step (4), in the second polynucleotide ligation molecule the terminal nucleotide of the synthesis strand occupies position n+1, wherein position n+1 is the nucleotide position which is occupied by the first nucleotide to be added to the terminal end of the second strand of the scaffold polynucleotide in step (4) and will be paired with the second nucleotide which was added to the terminal end of the first strand in step (2); the penultimate nucleotide of the synthesis strand occupies position n+2, wherein position n+2 is the nucleotide position which is occupied by the second nucleotide to be added to the terminal end of the second strand of the scaffold polynucleotide in step (4); the universal nucleotide occupies position n+4+x in the synthesis strand and is paired with a partner nucleotide in the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand; and wherein x is a number of nucleotide positions relative to position n+4 in the direction distal to the complementary ligation end and wherein the number is a whole number from 1 to 10 or more; and
(ii) in step (5) the second strand of the ligated scaffold polynucleotide is cleaved between positions n+2 and n+3.

14. A method according to claim 10, wherein:
(A) in step (1) the double-stranded scaffold polynucleotide is provided with a blunt end, with the terminal nucleotide of the second strand paired with the terminal nucleotide of the first strand;
(B) in step (2), in the first polynucleotide ligation molecule the terminal nucleotide of the synthesis strand occupies position n and is paired with the terminal nucleotide of the helper strand, wherein position n is the nucleotide position which is occupied by the first nucleotide to be added to the terminal end of the first strand of the scaffold polynucleotide in step (2); the universal nucleotide is the penultimate nucleotide of the synthesis strand, occupies position n+1 and is paired with the penultimate nucleotide of the helper strand; the terminal nucleotide of the helper strand is a non-ligatable nucleotide; and the complementary ligation end is provided with a blunt end;
(C) in step (3) the first strand of the ligated scaffold polynucleotide is cleaved between positions n and n+1 whereupon the universal nucleotide is removed from the scaffold polynucleotide and the first nucleotide of the first polynucleotide ligation molecule is retained in the scaffold polynucleotide, and whereupon a single base overhang is created in the scaffold polynucleotide with the terminal nucleotide of the first strand overhanging the terminal nucleotide of the second strand;
(D) in step (4), in the second polynucleotide ligation molecule the terminal nucleotide of the synthesis strand occupies position n, wherein position n is the nucleotide position which is occupied by the first nucleotide to be added to the terminal end of the second strand of the scaffold polynucleotide in step (4) and will be paired with the first nucleotide which was added to the terminal end of the first strand in step (2); the universal nucleotide is the penultimate nucleotide of the synthesis strand, occupies position n+1 and is paired with the terminal nucleotide of the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand; and
(E) in step (5) the second strand of the ligated scaffold polynucleotide is cleaved between positions n and n+1 whereupon the universal nucleotide is removed from the scaffold polynucleotide and the first and second nucleotides of the second polynucleotide ligation molecule are retained in the scaffold polynucleotide, and whereupon a blunt end is created in the scaffold polynucleotide with the terminal nucleotide of the second strand paired with the terminal nucleotide of the first strand.

15. A method according to claim 10, wherein:
(A) in step (1) the double-stranded scaffold polynucleotide is provided with a blunt end, with the terminal nucleotide of the second strand paired with the terminal nucleotide of the first strand;
(B) in step (2), in the first polynucleotide ligation molecule the terminal nucleotide of the synthesis strand occupies position n and is paired with the terminal nucleotide of the helper strand, wherein position n is the nucleotide position which is occupied by the first nucleotide to be added to the terminal end of the first strand of the scaffold polynucleotide in step (2); the universal nucleotide is the penultimate nucleotide of the synthesis strand, occupies position n+1 and is paired with the penultimate nucleotide of the helper strand; the terminal nucleotide of the helper strand is a non-ligatable nucleotide; and the complementary ligation end is provided with a blunt end;
(C) in step (3) the first strand of the ligated scaffold polynucleotide is cleaved between positions n and n+1 whereupon the universal nucleotide is removed from the scaffold polynucleotide and the first nucleotide of the first polynucleotide ligation molecule is retained in the scaffold polynucleotide, and whereupon a single base overhang is created in the scaffold polynucleotide with the terminal nucleotide of the first strand overhanging the terminal nucleotide of the second strand;
(D) in step (4), in the second polynucleotide ligation molecule the terminal nucleotide of the synthesis strand occupies position n, wherein position n is the nucleotide position which is occupied by the first nucleotide to be added to the terminal end of the second strand of the scaffold polynucleotide in step (4) and will be paired with the first nucleotide which was added to the terminal end of the first strand in step (2); the universal nucleotide occupies position n+2 in the synthesis strand and is paired with the penultimate nucleotide of the helper strand; the terminal nucleotide of the helper strand is a non-ligatable nucleotide, occupies position n+1 and is paired with the penultimate nucleotide of the synthesis strand; and the complementary ligation end is provided with a single base overhang, with the terminal nucleotide of the synthesis strand overhanging the terminal nucleotide of the helper strand; and
(E) in step (5) the second strand of the ligated scaffold polynucleotide is cleaved between positions n and n+1 whereupon the universal nucleotide is removed from the scaffold polynucleotide and the first and second nucleotides of the second polynucleotide ligation molecule are retained in the scaffold polynucleotide, and whereupon a blunt end is created in the scaffold polynucleotide with the terminal nucleotide of the second strand paired with the terminal nucleotide of the first strand.

16. A method according to claim 15, wherein:
(i) in step (4), in the second polynucleotide ligation molecule the terminal nucleotide of the synthesis strand occupies position n, wherein position n is the nucleotide position which is occupied by the first nucleotide to be added to the terminal end of the second strand of the scaffold polynucleotide in step (4) and will be paired with the first nucleotide which was added to the terminal end of the first strand in step (2); the universal nucleotide occupies position n+2+x in the synthesis strand and is paired with a partner nucleotide in the helper strand; the terminal nucleotide of the helper strand is a non-ligatable nucleotide, occupies position n+1 and is paired with the penultimate nucleotide of the synthesis strand; and the complementary ligation end is provided with a single base overhang, with the terminal nucleotide of the synthesis strand overhanging the terminal nucleotide of the helper strand; and wherein x is a number of nucleotide positions relative to position n+2 in the direction distal to the complementary ligation end and wherein the number is a whole number from 1 to 10 or more; and
(ii) in step (5) the second strand of the ligated scaffold polynucleotide is cleaved between positions n and n+1.

17. A method according to claim 10, wherein:
(A) in step (1) the double-stranded scaffold polynucleotide is provided with a blunt end, with the terminal nucleotide of the second strand paired with the terminal nucleotide of the first strand;
(B) in step (2), in the first polynucleotide ligation molecule the terminal nucleotide of the synthesis strand occupies position n and is paired with the terminal nucleotide of the helper strand, wherein position n is the nucleotide position which is occupied by the first nucleotide to be added to the terminal end of the first strand of the scaffold polynucleotide in step (2); the universal nucleotide occupies position n+2 in the synthesis strand and is paired with the nucleotide in the helper strand which is next to the penultimate nucleotide of the helper strand in the direction distal to the complementary ligation end; the terminal nucleotide of the helper strand is a non-ligatable nucleotide; and the complementary ligation end is provided with a blunt end;
(C) in step (3) the first strand of the ligated scaffold polynucleotide is cleaved between positions n and n+1 whereupon the universal nucleotide is removed from the scaffold polynucleotide and the first nucleotide of the first polynucleotide ligation molecule is retained in the scaffold polynucleotide, and whereupon a single base overhang is created in the scaffold polynucleotide with the terminal nucleotide of the first strand overhanging the terminal nucleotide of the second strand;
(D) in step (4), in the second polynucleotide ligation molecule the terminal nucleotide of the synthesis strand occupies position n, wherein position n is the nucleotide position which is occupied by the first nucleotide to be added to the terminal end of the second strand of the scaffold polynucleotide in step (4) and will be paired with the first nucleotide which was added to the terminal end of the first strand in step (2); the universal nucleotide is the penultimate nucleotide of the synthesis strand, occupies position n+1 and is paired with the terminal nucleotide of the helper strand; the terminal nucleotide of the helper strand is a non-ligatable nucleotide; and the complementary ligation end is provided with a single base overhang, with the terminal nucleotide of the synthesis strand overhanging the terminal nucleotide of the helper strand; and
(E) in step (5) the second strand of the ligated scaffold polynucleotide is cleaved between positions n and n+1 whereupon the universal nucleotide is removed from the scaffold polynucleotide and the first and second nucleotides of the second polynucleotide ligation molecule are retained in the scaffold polynucleotide, and whereupon a blunt end is created in the scaffold polynucleotide with the terminal nucleotide of the second strand paired with the terminal nucleotide of the first strand.

18. A method according to claim 17, wherein:
(i) in step (2), in the first polynucleotide ligation molecule the terminal nucleotide of the synthesis strand occupies position n and is paired with the terminal nucleotide of the helper strand, wherein position n is the nucleotide position which is occupied by the first nucleotide to be added to the terminal end of the first strand of the scaffold polynucleotide in step (2); the universal nucleotide occupies position n+2+x in the synthesis strand and is paired with the nucleotide in the helper strand which is next to the penultimate nucleotide of the helper strand in the direction distal to the complementary ligation end; the terminal nucleotide of the helper strand is a non-ligatable nucleotide; and the complementary ligation end is provided with a blunt end; and wherein x is a number of nucleotide positions relative to position n+2 in the direction distal to the complementary ligation end and wherein the number is a whole number from 1 to 10 or more; and
(ii) in step (3) the first strand of the ligated scaffold polynucleotide is cleaved between positions n and n+1.

19. A method according to claim 11, wherein: in step (2) the universal nucleotide is positioned in the synthesis strand of the first polynucleotide ligation molecule at position n+x and wherein in step (3) the ligated first strand of the scaffold polynucleotide is cleaved between positions n+2 and n+1, wherein x is a number of nucleotide positions relative to position n in the direction distal to the complementary ligation end and wherein x is a whole number from 2 to 10 or more.

20. A method according to claim 11, wherein in step (4) the universal nucleotide is positioned in the synthesis strand of the second polynucleotide ligation molecule at position n+x and wherein in step (5) the ligated second strand of the scaffold polynucleotide is cleaved between positions n+3 and n+2, wherein x is a number of nucleotide positions relative to position n in the direction distal to the complementary ligation end and wherein x is a whole number from 3 to 10 or more.

21. A method according to claim 11, wherein: in step (2) the universal nucleotide is positioned in the synthesis strand of the first polynucleotide ligation molecule at position n+x and wherein in step (3) the ligated first strand of the scaffold polynucleotide is cleaved between positions n+2 and n+1, and wherein in step (4) the universal nucleotide is positioned in the synthesis strand of the second polynucleotide ligation molecule at position n+x and wherein in step (5) the ligated second strand of the scaffold polynucleotide is cleaved between positions n+3 and n+2, wherein x is a number of nucleotide positions relative to position n in the direction distal to the complementary ligation end, and wherein in steps (2) and (3) x is a whole number from 2 to 10 or more and in steps (4) and (5) x is a whole number from 3 to 10 or more.

22. A method according to claim 14, wherein: in step (2) the universal nucleotide is positioned in the synthesis strand of the first polynucleotide ligation molecule at a position defined by the formula n+x and wherein in step (3) the ligated first strand of the scaffold polynucleotide is cleaved between positions n+1 and n, wherein x is a number of nucleotide positions relative to position n in the direction distal to the complementary ligation end and wherein x is a whole number from 1 to 10 or more.

23. A method according to claim 14, wherein in step (4) the universal nucleotide is positioned in the synthesis strand of the second polynucleotide ligation molecule at a position defined by the formula n+x and wherein in step (5) the ligated second strand of the scaffold polynucleotide is cleaved between positions n+1 and n, wherein x is a number of nucleotide positions relative to position n in the direction distal to the complementary ligation end and wherein x is a whole number from 1 to 10 or more.

24. A method according to claim 14, wherein: in step (2) the universal nucleotide is positioned in the synthesis strand of the first polynucleotide ligation molecule at a position defined by the formula n+x, wherein in step (3) the ligated first strand of the scaffold polynucleotide is cleaved between positions n+1 and n, wherein x is a whole number from 1 to 10 or more; and wherein in step (4) the universal nucleotide is positioned in the synthesis strand of the second polynucleotide ligation molecule at a position defined by the formula n+x and wherein in step (5) the ligated second strand of the scaffold polynucleotide is cleaved between positions n+1 and n wherein x is a whole number from 1 to 10 or more; and wherein in steps (2) and (4) x is a number of nucleotide positions relative to position n in the direction distal to the complementary ligation end.

25. A method according to claim 14, wherein: in step (2) the universal nucleotide is positioned in the synthesis strand of the first polynucleotide ligation molecule at a position defined by the formula n+1+x, and wherein in step (3) the ligated first strand of the scaffold polynucleotide is cleaved between positions n+1+x and n+x, wherein x is a number of nucleotide positions relative to position n in the direction distal to the complementary ligation end and wherein x is a whole number from 1 to 10 or more;

26. A method according to claim 14, wherein in step (4) the universal nucleotide is positioned in the synthesis strand of the second polynucleotide ligation molecule at a position defined by the formula n+1+x, and wherein in step (5) the ligated second strand of the scaffold polynucleotide is cleaved between positions n+1+x and n+x, wherein x is a number of nucleotide positions relative to position n in the direction distal to the complementary ligation end and wherein x is a whole number from 1 to 10 or more.

27. A method according to claim 14, wherein: in step (2) the universal nucleotide is positioned in the synthesis strand of the first polynucleotide ligation molecule at a position defined by the formula n+1+x, and wherein in step (3) the ligated first strand of the scaffold polynucleotide is cleaved between positions n+1+x and n+x, wherein x is a whole number from 1 to 10 or more; and wherein in step (4) the universal nucleotide is positioned in the synthesis strand of the second polynucleotide ligation molecule at a position defined by the formula n+1+x, and wherein in step (5) the ligated second strand of the scaffold polynucleotide is cleaved between positions n+1+x and n+x, wherein x is a whole number from 1 to 10 or more; and wherein in steps (2) and (4) x is a number of nucleotide positions relative to position n in the direction distal to the complementary ligation end.

28. A method according to claim 14, wherein in step (2) the universal nucleotide is positioned in the synthesis strand of the first polynucleotide ligation molecule at a position defined by the formula n+1+x, wherein in step (3) the ligated first strand of the scaffold polynucleotide is cleaved between positions n+x and n+x−1, wherein x is a number of nucleotide positions relative to position n in the direction distal to the complementary ligation end and wherein x is a whole number from 1 to 10 or more.

29. A method according to claim 14, wherein in step (4) the universal nucleotide is positioned in the synthesis strand of the second polynucleotide ligation molecule at a position defined by the formula n+1+x, wherein in step (5) the ligated second strand of the scaffold polynucleotide is cleaved between positions n+x and n+x−1, wherein x is a number of nucleotide positions relative to position n in the direction distal to the complementary ligation end and wherein x is a whole number from 1 to 10 or more

30. A method according to claim 14, wherein: in step (2) the universal nucleotide is positioned in the synthesis strand of the first polynucleotide ligation molecule at a position defined by the formula n+1+x, wherein in step (3) the ligated first strand of the scaffold polynucleotide is cleaved between positions n+x and n+x−1, wherein x is a whole number from 1 to 10 or more; and in step (4) the universal nucleotide is positioned in the synthesis strand of the second polynucleotide ligation molecule at a position defined by the formula n+1+x, wherein in step (5) the ligated second strand of the scaffold polynucleotide is cleaved between positions n+x and n+x−1, wherein x is a whole number from 1 to 10 or more; and wherein in steps (2) and (4) x is a number of nucleotide positions relative to position n in the direction distal to the complementary ligation end.

31. A method according to any one of claims 11, 12, 14, 15, 17, 25, 26 and 27 wherein
i. in steps (3) and/or (5) of the method of claims 11, 25, 26 and 27;
ii. in step (3) of the method of claim 12;
iii. in steps (3) and/or (5) of the method of claim 14;
iv. in step (3) of the method of claim 15; and
v. in step (5) of the method of claim 17;
in any one, more or all cycles of synthesis the cleavage step comprises a two step cleavage process comprising a first step comprising removing the universal nucleotide thus forming an abasic site, and a second step comprising cleaving the support strand at the abasic site.

32. A method according to claim 31, wherein the first step is performed with a nucleotide-excising enzyme.

33. A method according to claim 32, wherein the nucleotide-excising enzyme is a 3-methyladenine DNA glycosylase enzyme.

34. A method according to claim 33, wherein the nucleotide-excising enzyme is:
i. human alkyladenine DNA glycosylase (hAAG); or
ii. uracil DNA glycosylase (UDG).

35. A method according to any one of claims 31 to 34, wherein the second step is performed with a chemical which is a base.

36. A method according to claim 37, wherein the base is NaOH.

37. A method according to any one of claims 31 to 34, wherein the second step is performed with an organic chemical having abasic site cleavage activity.

38. A method according to claim 37, wherein the organic chemical is N,N′-dimethylethylenediamine.

39. A method according to any one of claims 31 to 34, wherein the second step is performed with an enzyme having abasic site lyase activity, optionally wherein the enzyme having abasic site lyase activity is.
(iv) AP Endonuclease 1;
(v) Endonuclease III (Nth); or
(vi) Endonuclease VIII.

40. A method according to any one of claims 11, 12, 13, 14, 15, 16, 19, 25, 26 and 27, wherein in any one, more or all cycles of synthesis cleavage step (3) comprises a one step cleavage process comprising removing the universal nucleotide with a cleavage enzyme; and/or a method according to any one of claims 11, 14, 17, 18, 19 and 20, wherein in any one, more or all cycles of synthesis cleavage step (5) comprises a one step cleavage process comprising removing the universal nucleotide with a cleavage enzyme; wherein the enzyme is:
(v) Endonuclease III;
(vi) Endonuclease VIII;
(vii) formamidopirimidine DNA glycosylase (Fpg); or
(viii) 8-oxoguanine DNA glycosylase (hOGG1).

41. A method according to any one of claims 12, 15, 17, 28, 29 and 30 wherein
i. in step (5) of the method of claim 12;
ii. in step (5) of the method of claim 15; and
iii. in step (3) of the method of claim 17;
iv. in steps (3) and/or (5) of the method of any one of claims 28, 29 and 30;
in any one, more or all cycles of synthesis the cleavage step comprises cleaving the support strand with an enzyme.

42. A method according to claim 41, wherein the enzyme is Endonuclease V.

43. A method according to any one of claims 13, 16 18, and 19-30; wherein in any one, more or all cycles of synthesis cleavage step (3) and/or cleavage step (5) comprises cleaving the support strand with an enzyme.

44. A method according to any one of claims 11 to 16, wherein in step (1) the terminal nucleotide of the second strand of the scaffold polynucleotide is the 5′ end of the second strand; in step (2) the terminal nucleotide of the synthesis strand of the first polynucleotide ligation molecule is the 5′ end of the synthesis strand; in step (3) the terminal nucleotide of the first strand of the scaffold polynucleotide is the 3′ end of the first strand; and in step (4) the terminal nucleotide of the synthesis strand of the second polynucleotide ligation molecule is the 3′ end of the synthesis strand.

45. A method according to any one of claim 14, 17 or 18, wherein in step (1) the terminal nucleotide of the second strand of the scaffold polynucleotide is the 3′ end of the second strand; in step (2) the terminal nucleotide of the synthesis strand of the first polynucleotide ligation molecule is the 3′ end of the synthesis strand; in step (3) the terminal nucleotide of the first strand of the scaffold polynucleotide is the 5′ end of the first strand; and in step (4) the terminal nucleotide of the synthesis strand of the second polynucleotide ligation molecule is the 5′ end of the synthesis strand.

46. A method according to any one of the preceding claims, wherein in any one, more or all cycles of synthesis one or more of the nucleotides which are incorporated into one strand of a double-stranded polynucleotide forms a pair with a partner nucleotide at the corresponding position in the opposite strand, and wherein nucleotides of a pair are complementary nucleotides, preferably naturally complementary nucleotides.

47. A method according to any one of the preceding claims, wherein in any one, more or all cycles of synthesis, prior to cleavage steps (3) and (5) the helper strand is removed from the ligated scaffold polynucleotide.

48. A method according to claim 47, wherein the helper strand is removed from the scaffold polynucleotide by: (i) heating the scaffold polynucleotide to a temperature of about 80° C. to about 95° C. and separating the helper strand from the scaffold polynucleotide, (ii) treating the scaffold polynucleotide with urea solution, such as 8M urea and separating the helper strand from the scaffold polynucleotide, (iii) treating the scaffold polynucleotide with formamide or formamide solution, such as 100% formamide and separating the helper strand from the scaffold polynucleotide, or (iv) contacting the scaffold polynucleotide with a single-stranded polynucleotide molecule which comprises a region of nucleotide sequence which is complementary with the sequence of the helper strand, thereby competitively inhibiting the hybridisation of the helper strand to the scaffold polynucleotide.

49. A method according to any one of the preceding claims, wherein both strands of the synthesised double-stranded polynucleotide are DNA strands.

50. A method according to claim 49, wherein incorporated nucleotides are dNTPs.

51. A method according to any one of claims 1 to 50, wherein one strand of the synthesised double-stranded polynucleotide is a DNA strand and the other strand of the synthesised double-stranded polynucleotide is an RNA strand.

52. A method according to claim 51, wherein nucleotides incorporated into an RNA strand are NTPs.

53. A method according to any one of the preceding claims, wherein the ligase enzyme is a T3 DNA ligase or a T4 DNA ligase.

54. A method according to any one of the preceding claims, further comprising further extending the first and/or second strands of the scaffold polynucleotide following cleavage step (3) and/or cleavage step (5) by the action of a polymerase enzyme and/or a transferase enzyme.

55. A method according to claim 54, wherein the polymerase enzyme is a DNA polymerase, preferably a modified DNA polymerase having an enhanced ability to incorporate a dNTP comprising a reversible terminator group compared to an unmodified polymerase.

56. A method according to claim 55, wherein the polymerase is a variant of the native DNA polymerase from Thermococcus species 9° N, preferably species 9° N-7.

57. A method according to claim 56, wherein one or more of the nucleotides incorporated by the polymerase are dNTPs comprising a reversible terminator group.

58. A method according to claim 57, wherein one or more of the incorporated nucleotides comprising a reversible terminator group are 3′-O-allyl-dNTPs.

59. A method according to claim 57, wherein one or more of the incorporated nucleotides comprising a reversible terminator group are 3′-O-azidomethyl-dNTPs.

60. A method according to claim 54, wherein the polymerase enzyme is an RNA polymerase such as T3 or T7 RNA polymerase, optionally a modified RNA polymerase having an enhanced ability to incorporate an NTP comprising a reversible terminator group compared to an unmodified polymerase.

61. A method according to claim 60, wherein one or more of the nucleotides incorporated by the polymerase are dNTPs comprising a reversible terminator group.

62. A method according to claim 61, wherein one or more of the incorporated nucleotides comprising a reversible terminator group are 3′-O-allyl-dNTPs.

63. A method according to claim 61, wherein one or more of the incorporated nucleotides comprising a reversible terminator group are 3′-O-azidomethyl-dNTPs.

64. A method according to claim 54, wherein the transferase enzyme has a terminal transferase activity, optionally wherein the enzyme is a terminal nucleotidyl transferase, a terminal deoxynucleotidyl transferase, terminal deoxynucleotidyl transferase (TdT), pol lambda, pol mu or Φ29 DNA polymerase.

65. A method according to any one of claims 57 to 64, wherein the step of removing the reversible terminator group is performed with tris(carboxyethyl)phosphine (TCEP).

66. A method according to any one of claims 10 to 65, wherein in a cycle of synthesis, in a given ligation reaction at the complementary ligation end of the polynucleotide ligation molecule: (a) if the helper strand comprises a non-ligatable terminal nucleotide at the 3′ end of the helper strand, the nucleotide is a 2′,3′-dideoxynucleotide or a 2′-deoxynucleotide; or (b) if the helper strand comprises a non-ligatable terminal nucleotide at the 5′ end of the helper strand, the nucleotide lacks a phosphate group.

67. A method according to any one of the preceding claims, wherein in any one, more or all cycles of synthesis the first and second strands of the scaffold polynucleotide are connected by a hairpin loop at the end of the molecule opposite the ligation end.

68. A method according to any one of claims 10 to 67, wherein in any one, more or all cycles of synthesis in step (2) and/or in step (4) in the polynucleotide ligation molecule the synthesis strand and the helper strand hybridized thereto are connected by a hairpin loop at the end opposite the complementary ligation end.

69. A method according to claim 68, wherein in any one, more or all cycles of synthesis:
c) the first and second strands of the scaffold polynucleotide are connected by a hairpin loop at the end of the molecule opposite the ligation end; and
d) in step (2) and/or in step (4) in the polynucleotide ligation molecule the synthesis strand and the helper strand hybridized thereto are connected by a hairpin loop at the end opposite the complementary ligation end.

70. A method according to any one of the preceding claims, wherein the first and second strands of the scaffold polynucleotide are tethered to a common surface.

71. A method according to claim 70 wherein the first strand and/or the second strand comprises a cleavable linker, wherein the linkers may be cleaved to detach the double-stranded polynucleotide from the surface following synthesis.

72. A method according to claim 67, claim 68 or claim 69, wherein the hairpin loop in the scaffold polynucleotide is tethered to a surface.

73. A method according to claim 72 wherein the hairpin loop is tethered to a surface via a cleavable linker, wherein the linker may be cleaved to detach the double-stranded polynucleotide from the surface following synthesis.

74. A method according to claim 71 or claim 73, wherein the cleavable linker is a UV cleavable linker.

75. A method according to any one of claims 70 to 74, wherein the surface is a microparticle.

76. A method according to any one of claims 70 to 75, wherein the surface is a planar surface.

77. A method according to any one of claims 70 to 76, wherein the surface comprises a gel.

78. A method according to claim 77, wherein the surface comprises a polyacrylamide surface, such as about 2% polyacrylamide, preferably wherein the polyacrylamide surface is coupled to a solid support such as glass.

79. A method according to any one of claims 70 to 78, wherein the first and second strands of the scaffold polynucleotide are tethered to a common surface via one or more covalent bonds.

80. A method according to claim 79, wherein the one or more covalent bonds is formed between a functional group on the common surface and a functional group on the scaffold molecule, wherein the functional group on the scaffold molecule is an amine group, a thiol group, a thiophosphate group or a thioamide group.

81. A method according to claim 80, wherein the functional group on the common surface is a bromoacetyl group, optionally wherein the bromoacetyl group is provided on a polyacrylamide surface derived using N-(5-bromoacetamidylpentyl) acrylamide (BRAPA).

82. A method according to any one of the preceding claims, wherein synthesis cycles are performed in droplets within a microfluidic system.

83. A method according to claim 82, wherein the microfluidic system is an electrowetting system.

84. A method according to claim 83, wherein the microfluidic system is an electrowetting-on-dielectric system (EWOD).

85. A method according to any one of the preceding claims, wherein following synthesis the strands of the double-stranded polynucleotides are separated to provide a single-stranded polynucleotide having a predefined sequence.

86. A method according to any one of the preceding claims, wherein following synthesis the double-stranded polynucleotide or a region thereof is amplified, preferably by PCR.

87. A method of assembling a polynucleotide having a predefined sequence, the method comprising performing the method of any one of the preceding claims to synthesise a first polynucleotide having a predefined sequence and one or more additional polynucleotides having a predefined sequence and joining together the first and one or more additional polynucleotides.

88. A method according to claim 87 wherein the first polynucleotide and the one or more additional polynucleotides are double-stranded.

89. A method according to claim 88 wherein the first polynucleotide and the one or more additional polynucleotides are single-stranded.

90. A method according to any one of claims 87 to 89, wherein the first polynucleotide and the one or more additional polynucleotides are cleaved to create compatible termini and joined together, preferably by ligation.

91. A method according to claim 90, wherein the first polynucleotide and the one or more additional polynucleotides are cleaved by a restriction enzyme at a cleavage site.

92. A method according to any one of claims 82 to 91, wherein the synthesis and/or assembly steps are performed in droplets within a microfluidic system.

93. A method according to claim 92 wherein the assembly steps comprise providing a first droplet comprising a first synthesised polynucleotide having a predefined sequence and a second droplet or a plurality of further droplets each comprising an additional one or more synthesised polynucleotides having a predefined sequence, wherein the droplets are brought in contact with each other and wherein the synthesised polynucleotides are joined together thereby assembling a polynucleotide comprising the first and additional one or more polynucleotides.

94. A method according to claim 93 wherein the synthesis steps are performed by providing a plurality of droplets each droplet comprising reaction reagents corresponding to a step of the synthesis cycle, and sequentially delivering the droplets to the scaffold polynucleotide in accordance with the steps of the synthesis cycles.

95. A method according to claim 94, wherein following delivery of a droplet and prior to the delivery of a next droplet, a washing step is carried out to remove excess reaction reagents.

96. A method according to claims 94 and 95, wherein the microfluidic system is an electrowetting system.

97. A method according to claim 96, wherein the microfluidic system is an electrowetting-on-dielectric system (EWOD).

98. A method according to any one of claims 93 to 97, wherein synthesis and assembly steps are performed within the same system.

99. A method of storing data in a polynucleotide molecule, the method comprising: (a) performing a first series of extension reactions by extending one strand of a double-stranded polynucleotide and then extending the opposite strand by a method according to any one of claims 1 to 98, thereby extending the polynucleotide molecule by one or more pairs of nucleotides to generate a first nucleotide sequence; and (b) performing one or more further series of extension reactions by further extending one strand of the double-stranded polynucleotide and then further extending the opposite strand by a method according to any one of claims 1 to 98, thereby extending the polynucleotide molecule by one or more further pairs of nucleotides, to generate a second or further nucleotide sequence in the polynucleotide, wherein generated sequences are indicative of information encoded into the extended polynucleotide molecule.

100. A method of storing data in bit form in a polynucleotide molecule, the method comprising: (a) performing a first series of extension reactions by extending one strand of a double-stranded polynucleotide and then extending the opposite strand by a method according to any one of claims 1 to 98, thereby extending the polynucleotide molecule by one or more pairs of nucleotides to generate a first nucleotide sequence in the polynucleotide molecule indicative of a first bit of information; and (b) performing one or more further series of extension reactions by further extending one strand of the double-stranded polynucleotide and then further extending the opposite strand by a method according to any one of claims 1 to 98, thereby extending the polynucleotide molecule by one or more further pairs of nucleotides to generate further nucleotide sequences in the polynucleotide molecule indicative of one or more further bits of information.

101. A method of storing data in digital form in a polynucleotide molecule, the method comprising: (a) performing a first series of extension reactions by extending one strand of a double-stranded polynucleotide and then extending the opposite strand by a method according to any one of claims 1 to 98, thereby extending the polynucleotide molecule by one or more pairs of nucleotides to generate a first nucleotide sequence in the polynucleotide molecule indicative of the “0” or “1” state of a bit of digital information; and (b) performing one or more further series of extension reactions by further extending one strand of the double-stranded polynucleotide and then further extending the opposite strand by a method according to any one of claims 1 to 98, thereby extending the polynucleotide molecule by one or more further pairs of nucleotides to generate a second nucleotide sequence in the polynucleotide molecule indicative of the opposite state of the bit to that generated in step (a).

102. A method according to claim 101, comprising repeating steps (a) and (b) multiple times to generate nucleotide sequences indicative of multiple bits of digital information.

103. A method of making a polynucleotide microarray, wherein the microarray comprises a plurality of reaction areas, each area comprising one or more polynucleotides having a predefined sequence, the method comprising:
c) providing a surface comprising a plurality of reaction areas, each area comprising one or more double-stranded anchor or scaffold polynucleotides, and
d) performing cycles of synthesis according to the method of any one of claims 1 to 97 at each reaction area, thereby synthesising at each area one or more double-stranded polynucleotides having a predefined sequence.

104. A method according to claim 103, wherein following synthesis the strands of the double-stranded polynucleotides are separated, whereupon each area of the microarray comprises one or more single-stranded polynucleotides having a predefined sequence.

105. A polynucleotide synthesis system for carrying out the method according to any one of claims 1 to 104, the system comprising: (a) an array of reaction areas, wherein each reaction area comprises at least one scaffold polynucleotide; and (b) means for the delivery of the reaction reagents to the reaction areas; and optionally, (c) means to cleave the synthesised double-stranded polynucleotide from the scaffold polynucleotide.

106. A system according to claim 105 further comprising means for providing the reaction reagents in droplets and means for delivering the droplets to the scaffold polynucleotide in accordance with the synthesis cycles.

107. A kit for use with the system of claim 105 or 106 and for carrying out the method according to any one of claims 1 to 102, the kit comprising volumes of reaction reagents corresponding to the steps of the synthesis cycles.
